(19) United States
(12) Patent Application Publication
(10) Pub. No.: US 2023/0158088 A1
(43) Pub. Date: May 25, 2023

(54) COMPOSITIONS FOR MODULATING GUT MICROFLORA POPULATIONS, ENHANCING DRUG POTENCY AND TREATING VIRAL INFECTIONS, AND METHODS FOR MAKING AND USING SAME

(71) Applicant: PERSEPHONE BIOSCIENCES, INC., San Diego, CA (US)

(72) Inventors: Stephanie J. CULLER, Del Mar, CA (US); Robert J. HASELBECK, San Diego, CA (US); Stephen VAN DIEN, San Diego, CA (US)

(21) Appl. No.: 17/914,703

(22) PCT Filed: Mar. 26, 2021
(86) PCT No.: PCT/US2021/024503

§ 371 Date: Sep. 26, 2022

Related U.S. Application Data
(60) Provisional application No. 63/000,369, filed on Mar. 26, 2020.

(51) Int. Cl. A61K 35/747; A61K 35/745; A61K 35/744; A61K 35/742; A61K 35/741; A61P 35/00; A61P 31/12

(52) U.S. Cl. CPC A61K 35/747; A61K 35/745; A61K 35/744; A61K 35/742; A61K 35/741; A61P 35/00; A61P 31/12

(57) ABSTRACT

Provided are compositions, including products of manufacture and kits, and methods, comprising combinations of microbes, such as non-pathogenic, live bacteria and/or bacterial spores, for the control, amelioration, prevention, and treatment of a disease or condition, for example, a viral infection such as a COVID19 infection, and these non-pathogenic, live bacteria and/or bacterial spores can be administered to an individual, thereby resulting in a modification or modulation of the individual's gut microfloral population(s), and by modulating or modifying the individual's gut microbial population(s) using compositions, products of manufacture and methods as provided herein, the pharmacodynamics or effectiveness of a drug or a vaccine administered to the individual is altered, for example, the pharmacodynamics of the drug or vaccine is enhanced, the individual's ability to absorb a drug is modified or the dose efficacy of a drug or vaccine is increased.

### TECHNICAL FIELD

This invention generally relates to microbiology, pharmacology and antiviral therapies. In alternative embodiments, provided are compositions, including products of manufacture and kits, and methods, comprising combinations or consortia of microbes, such as non-pathogenic, live bacteria and/or bacterial spores, for the control, amelioration, prevention, and treatment of a disease or condition, for example, a viral infection such as a COVID19 infection. In alternative embodiment, these non-pathogenic, live bacteria and/or bacterial spores are administered to an individual in need thereof, thereby resulting in a modification or modulation of the individual's gut microfloral population(s). In alternative embodiments, by modulating or modifying the individual's gut microbial population(s) using compositions, products of manufacture and methods as provided herein, the pharmacodynamics of a drug or a vaccine, for example, antimicrobial such as an anti-bacterial, an antiviral or an antimalarial drug or a vaccine, administered to the individual is altered, for example, the pharmacodynamics of the drug is enhanced, for example, the individual's ability to absorb a drug is modified (for example, accelerated or slowed, or enhanced), or the dose efficacy of a drug is increased (for example, resulting in the requirement for a lower dose of drug to provide an intended effect), which can result in lowering the effective toxicity of the drug; or, alternatively the efficacy of a vaccine is enhanced or there are fewer or diminished side effects or negative reactions to the vaccine, for example, a diminished unwanted reaction to a vaccine carrier such as a liposome or nanolipid particle. For example, in alternative embodiments, the modulating or modifying of the individual's gut microbial population(s) increases the dose efficacy of the antimicrobial, for example, anti-bacterial, antiviral or antimalarial drug, thereby controlling, ameliorating, preventing and/or treating of that viral infection. In alternative embodiments, the amount, identity, presence, and/or ratio of gut microbiota in a subject is manipulated to facilitate one or more co-treatments, for example, in alternative embodiments, the combinations of microbes as provided herein are administered with an antimicrobial, for example, antibacterial, antiviral or antimalarial, therapy, which can comprise a drug, a small molecule, a vaccine, an antibody, a cell therapy, a natural killer (NK) cell therapy, angiotensin II receptor blockers, a defensin-mimetic, a nanobody, a peptide, an immune modulator, an immunotherapy, an anti-necrosis, a nucleoside, a quinoline compound, a protease inhibitor, a sphingosine kinase-2 (SK2) inhibitor, an interleukin receptor antagonist, a nanoviricide or other antimicrobial treatments.

### BACKGROUND

The Coronavirus (SARS)-CoV-2, or COVID-19, and the potentially acute and life-threatening disease that it can cause, has quickly become a global pandemic (Rothan and Byrareddy 2020). SARS-CoV-2 is the seventh-known coronavirus to infect people (after 229E, NL63, OC43, HKU1, MERS, and SARS). Since its original discovery in China in December 2019, COVID-19 is now established in many countries and has caused immense disruption in social order, economic institutions, and is causing a severe strain on hospitals and clinics as more and more people with severe symptoms are seeking care. There are at present no specific antiviral drugs against COVID-19 infection, although drugs effective against other viruses like nucleoside analogs and HIV inhibitors could help treat COVID-19 infection and symptoms until new drugs become available (Wang et al. (2020) Journal of Medical Virology 92 (4): 441-47; Lu et al. (2020) BioScience Trends 14 (1). https://doi.org/10.5582/bst.2020.01020). In addition to these potential treatments and as the COVID-19 virus spreads, new and novel approaches for prevention of morbidity and mortality caused by this disease are desperately needed.

As of March 2021, several advances in the clinical treatment of (SARS)-CoV-2 have become available or are in late-stage clinical trials (Izda et al (2021) Clinical Immunology 222:108634). For instance, the adenosine nucleotide analog drug remdesivir, originally developed as an anti-viral drug against RNA viruses like Ebola, shows efficacy in the clinic for reducing morbidity/mortality and shortening the time of recovery of hospitalized COVID-19 patients (Beigel et al (2020) New England Journal of Medicine 383:1826). The REGN-COV2 antibody cocktail therapeutic developed by Regeneron Pharmaceuticals is a combination of antibodies specific to different parts of the SARS-CoV-2 spike protein and is shown to reduce viral load compared to placebo (Weinreich et al (2021) New England Journal of Medicine 384:238). Medications such as the Janus Kinase 1/2 inhibitor ruxolitinib (Rosee et al (2020) Leukemia 34:1805), the anti-IL-6 monoclonal antibody tocilizumab (Rosas et al (2021) New England J. of Med.), and corticosteroids such as dexamethasone (Horby et al (2021) New England J. of Med. 384:693) are intended to reduce hyperimmune responses that lead to cytokine storm in advanced hospitalized COVID-19 patients (Izda et al (2021) Clinical Immunology 222:108634).

In addition to immediate therapeutic treatments for COVID-19 related disease symptoms, there are now several highly effective and clinically available vaccines available that are directed against the spike protein against the (SARS)-CoV-2 coronavirus. For instance, the mRNA-based vaccines against the (SARS)-CoV-2 spike protein vaccines developed by Moderna (Baden et al (2021) New England J. Med.) 384:403) and by Pfizer (Polack et al (2020) 383:2603) show 94.1% and 95% efficacy, respectively, at preventing COVID-19 illness, including severe illness, in vaccinated individuals. The adenovirus-based DNA vector vaccines against the SARS-CoV-2 spike protein developed by Johnson and Johnson (Sadoff et al (2021) New England J. Med.) and by AstraZeneca (Madhi et al (2021) New England J. Med.) are also highly effective against SARS-CoV-2 and its variants. However, the current vaccines also have a number of side effects, and prevention of these or reduction in severity is desired.

### SUMMARY

In alternative embodiments, provided are methods for:
controlling, ameliorating or treating a viral infection such as a COVID-19 infection in an individual in need thereof,
lessoning the symptoms of or mortality of a viral infection,
enhancing the efficacy of an anti-viral drug, treatment, or a vaccine, or diminishing a side effects or a negative reaction to a vaccine, wherein optionally the vaccine is an antiviral vaccine, for example, diminishing an unwanted reaction to a vaccine carrier such as a liposome or nanolipid particle, or

enhancing the efficacy of a vaccine, or changing the gut microbiome in an individual in need thereof such that the individual has fewer or diminished side effects or negative reactions to an administered vaccine, wherein optionally the vaccine is an antiviral vaccine, and optionally the antiviral vaccine is an RNA-based vaccine, wherein optionally the RNA-based vaccine comprises RNA formulated in a liposome or a nanolipid particle,

the method comprising:

(a) administering or having administered to an individual in need thereof a formulation comprising at least two different species or genera (or types) of non-pathogenic bacteria, wherein each of the non-pathogenic bacteria comprise (or are in the form of) a plurality of non-pathogenic colony forming live bacteria, a plurality of non-pathogenic germinable bacterial spores, or a combination thereof; or,

(b) (i) providing a formulation comprising at least two different species or genera (or types) of non-pathogenic bacteria, wherein each of the non-pathogenic bacteria comprise (or are in the form of) a plurality of non-pathogenic colony forming live bacteria, a plurality of non-pathogenic germinable bacterial spores, or a combination thereof, and

(ii) administering or having administered to an individual in need thereof the formulation;

wherein the formulation comprises a or any combination of at least two different species or genera of non-pathogenic, live bacteria, or spore thereof, if the bacteria is spore forming, as described Tables 1, 4, 7, r 8 and/or 42, or live biotherapeutic compositions or combinations of bacteria as set forth in Tables 9 and/or 42,

and optionally the different species or genera (or types) of non-pathogenic, live bacteria or viable spores are present in approximately equal amounts, or each of the different species or genera (or types) of non-pathogenic, live bacteria or non-pathogenic germinable bacterial spores represent at least about 1%, 5%, 10%, 20%, 30%, 40%, or 50% or more, or between about 1% and 75%, of the total amount of non-pathogenic, live bacteria and non-pathogenic germinable bacterial spores in the formulation,

and optionally only or substantially only non-pathogenic, live bacteria are present in the formulation, or only or substantially only non-pathogenic germinable bacterial spores are present in the formulation, or approximately equal amounts of non-pathogenic, live bacteria and non-pathogenic germinable bacterial spores are present in the formulation.

In alternative embodiments of methods as provided herein:
the method further comprises administering or having administered an antimicrobial drug or therapy, for example, an antiviral, antibacterial or antimalarial, drug or treatment, or an anti-bacterial or anti-viral vaccine, wherein optionally the vaccine is an adenovirus-based DNA vector vaccine or an RNA (for example, mRNA)-based vaccine,

and optionally the antimicrobial drug or therapy is administered before, during (concurrently with) and/or after administration a formulation as provided herein, for example, a formulation comprising a combination of microbes (for example, viable bacteria and/or spores), as provided herein,

and optionally a formulation as provided herein comprises both a combination of microbes (for example, viable bacteria and/or spores) as provided herein and an antimicrobial drug, for example, an antiviral, antibacterial or antimalarial, drug,

and optionally the antimicrobial (for example, antiviral) drug comprises: lopinavir; ritonavir; oseltamivir (for example, TAMIFLU™); lopinavir combined (formulated) with ritonavir, or KALETRA™; chloroquine phosphate, chloroquine diphosphate, hydroxychloroquine (for example, PLAQUENIL™) or oral chloroquine (for example, ARALEN™); remdesivir (for example, GS-5734™, Gilead Sciences); nevirapine, efavirenz, emtricitabine, tenofovir (or the combination efavirenz with emtricitabine and tenofovir, or ATRIPLA™); amprenavir (for example, AGENERASE™); nelfinavir (for example, VIRACEPT™); a thiazolide class drug, optionally nitazoxanide (or ALINIA™, NIZONIDE™) or tizoxanide (or 2-Hydroxy-N-(5-nitro-2-thiazolyl)benzamide); plitidepsin (also known as dehydrodidemnin B), or APLIDIN™ (PharmaMar, S.A.); an inhibitor or S-phase kinase-associated protein 2 (SKP2), or dioscin, or niclosamide, or NICLOCIDE™, FENASAL™, or PHENASAL™; ribavirin; an interferon such as interferon alpha, interferon beta, interferon type I, interferon type II and/or interferon type III, or a combination of ribavirin and interferon beta, or a combination of lopinavir and ritonavir and interferon-beta-1b; abacavir, actemra, acyclovir for example, (ACICLOVIR™) adefovir, amantadine, rintatolimod (for example, AMPLIGEN™), amprenavir (for example, AGENERASE™), aprepitant, arbidol, atazanavir, balavir, baloxavir marboxil (XOFLUZA™), bepotastine, bevirimat, bictegravir, biktarvy, brilacidin, cidofovir, caspofungin, lamivudine and zidovudine (for example, COMBVIR™) cobicstat, colisitin, cocaine, darunavir, delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, lamivudine, lopinavir, loviride, ledipasvir, leronlimab, maraviroc, methisazone, moroxydine, nelfinavir, nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (for example, BCX4430, IMMUCILLIN-A™) remdesivir (for example, GS-5734™, Gilead Sciences), cytarabine, gemcitabine, emtricitabine, zalcitabine, stavudine, telbivudine, zidovudine, idoxuridine and/or trifluridine or any combination thereof), oseltamivir (or TAMIFLU™), peginterferon alfa-2a, penciclovir, peramivir (for example, RAPIVAB™), perfenazine, pleconaril, plurifloxacin, podophyllotoxin, pyramidine, raltegravir, rifampicin, ribavirin, rilpivirine, rimantadine, ritonavir, saquinavir, sofosbuvir, telaprevir, tegobuv, tenofovir alafenamide, tenofovir disoproxil, tenofovir, tipranavir, trifluridine, trizivir, tromantadine, truvada, valaciclovir (for example, VALTREX™), valganciclovir, valrubicin, vapreotide, vicriviroc, vidarabine, viramidine, velpatasvir, vivecon, zalcitabine, zanamivir (for example, RELENZA™) or zidovudine; a serine protease inhibitor, optionally camostat; an anti-PD-1 checkpoint inhibitor, optionally camrelizumab; a compound or antibody capable of binding complement factor C5 and blocking membrane attack complex formation, optionally eculizumab; a cathepsin inhibitor, optionally a cathepsin K, B or L inhibitor, optionally relacatib; thalidomide, or thalidomide and glucocorticoid (optionally low-dose glucocorticoid), or and thalidomide and celecoxib; an antibacterial antibiotic or a macrolide drug, wherein optionally the macrolide drug comprises azithromycin (for example, ZITHROMAX™, or AZITHROCIN™), clarithromycin (for example, BIAXIN™) erythromycin (for example, ERYTHROCIN™), or fidaxomicin (for example, DIFICID™ or DIFICLIR™), troleandomycin (for example, TEKMISIN™), tylosin (for example, TYLOCINE™ or TYLAN™), solithromycin (for example, SOLITHERA™), oleandomycin (or SIGMAMYCINE™), midecamycin, roxithromycin, kitasamycin or turimycin, josamycin, carbomycin or magnamycin, and/or spiramycin; opaganib or YELIVA™; or, any two, three or more or combination thereof,
the formulation comprises an inner core surrounded by an outer layer of polymeric material enveloping the inner core, wherein the non-pathogenic bacteria or the non-pathogenic germinable bacterial spores are substantially in the inner core, and optionally the polymeric material comprises a natural polymeric material;
the formulation is formulated or manufactured as or in: a nano-suspension delivery system; an encochleated formulation; or, as a multilayer crystalline, spiral structure with no internal aqueous space;
the formulation is formulated or manufactured as a delayed or gradual enteric release composition or formulation, and optionally the formulation comprises a gastro-resistant coating designed to dissolve at a pH of 7 in the terminal ileum, optionally an active ingredient is coated with an acrylic based resin or equivalent, optionally a poly(meth)acrylate, optionally a methacrylic acid copolymer B, NF, optionally EUDRAGIT S™ (Evonik Industries AG, Essen, Germany), which dissolves at pH 7 or greater, optionally comprises a multimatrix (MMX) formulation, and optionally manufactured as enteric coated to bypass the acid of the stomach and bile of the duodenum;
the plurality of non-pathogenic colony forming live bacteria are substantially dormant colony forming live bacteria, or the plurality of non-pathogenic colony forming live bacteria or the plurality of non-pathogenic germinable bacterial spores are lyophilized, wherein optionally the dormant colony forming live bacteria comprise live vegetative bacterial cells that have been rendered dormant by lyophilization or freeze drying in the absence of oxygen to maintain viability of strict anaerobic species;
the formulation comprises at least about 1×10⁴ colony forming units (CFUs), or between about 1×10¹ and 1×10¹³ CFUs, 1×10² and 1×10¹⁰ CFUs, 1×10² and 1×10⁸ CFUs, 1×10³ and 1×10⁷ CFUs, or 1×10⁴ and 1×10⁶ CFUs, of non-pathogenic live bacteria and/or non-pathogenic germinable bacterial spores;
the formulation comprises at least one (or any one, several, or all of) non-pathogenic bacteria or spore of the family or genus (or class): Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), or a combination thereof,
the formulation comprises at least one (or any one, several, or all of) non-pathogenic bacteria or spore form thereof as set forth in Tables 1, 4, 7 or 8, or included in the combination of non-pathogenic bacteria and/or spores thereof (or spore derived from) as set forth in Tables 9 and/or 42;
the formulation comprises combination of non-pathogenic bacteria and/or spores thereof (or spore derived from) as set forth in Tables 9 and/or 42;
the formulation comprises water, sterile water, saline, sterile saline, a pharmaceutically acceptable preservative, a carrier, a buffer, a diluent, an adjuvant or a combination thereof;
the formulation is administered orally or rectally, or is formulated and/or administered as a liquid, a food, a gel, a candy, an ice, a lozenge, a tablet, pill or capsule, or a suppository or as an enema formulation, or the formulation is administered as an or is in a form for intra-rectal or intra-colonic administration;
the formulation is administered to the individual in need thereof in one, two, three, or four or more doses, and wherein the one, two, three, four or five or more doses are administered on a daily basis (optionally once a day, bid or tid or more), every other day, every third day, or about once a week, and optionally the two, three, or four or more doses are administered at least a week apart (or dosages are separated by about a week);
a formulation or a method as provided herein further comprises or further comprises administration of: an antimicrobial drug, for example, an antiviral, antibacterial or antimalarial, drug, and optionally the antimicrobial (for example, antiviral) drug comprises: lopinavir; ritonavir; oseltamivir (for example, TAMIFLU™); lopinavir combined (formulated) with ritonavir, or KALETRA™; chloroquine phosphate (for example, RESOCHIN™), chloroquine diphosphate, hydroxychloroquine (for example, PLAQUENIL™) or oral chloroquine (for example, ARALEN™); remdesivir (for example, GS-5734™, Gilead Sciences); nevirapine, efavirenz, emtricitabine, tenofovir (or the combination efavirenz with emtricitabine and tenofovir, or ATRIPLA™); amprenavir (for example, AGENERASE™); nelfinavir (for example, VIRACEPT™); a thiazolide class drug, optionally nitazoxanide (or ALINIA™, NIZONIDE™) or tizoxanide (or 2-Hydroxy-N-(5-nitro-2-thiazolyl)benzamide); plitidepsin (also known as dehydrodidemnin B), or APLIDIN™ (PharmaMar, S.A.); an inhibitor or S-phase kinase-associated protein 2 (SKP2), or dioscin, or niclosamide, or NICLOCIDE™, FENASAL™, or PHENASAL™; ribavirin; an interferon such as interferon alpha, interferon beta, interferon type I, interferon type II and/or interferon type III, or a combination of ribavirin and interferon beta, or a combination of lopinavir and ritonavir and interferon-beta-1b; abacavir, actemra, acyclovir for example, (ACICLOVIR™) adefovir, amantadine, ampligen, amprenavir (for example, AGENERASE™) aprepitant, atazanavir, balavir, baloxavir marboxil (XOFLUZA™), bepotastine, bevirimat, bictegravir, biktarvy, brilacidin, cidofovir, caspofungin, lamivudine and zidovudine (for example, COMBVIR™), cobicstat, colisitin, cocaine, danoprevir or danoprevir and ritonavir (for example, GANOVO™) darunavir (or darunavir and cobicstat, for example, PREZCOBIX™), delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, ibacitabine, icatibant, idoxuridine, ifenprodil, imiquimod, imunovir, indinavir, inosine, lamivudine, lopinavir, loviride, ledipasvir, leronlimab, maraviroc, methisazone, moroxydine, nelfinavir, nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (for example, BCX4430 by Biocryst, IMMUCILLIN-A™), remdesivir (for example, GS-5734™, Gilead Sciences), cytarabine, gemcitabine, emtricitabine, zalcitabine, stavudine, telbivudine, zidovudine, idoxuridine and/or trifluridine or any combination thereof), oseltamivir (or TAMIFLU™), peginterferon alfa-2a, penciclovir, peramivir (for example, RAPIVAB™), perfenazine, pleconaril, plurifloxacin, podophyllotoxin, pyramidine, raltegravir, rifampicin, ribavirin, rilpivirine, rimantadine, ritonavir, saquinavir, sofosbuvir, telaprevir, tegobuv, tenofovir alafenamide, tenofovir disoproxil, tenofovir, tipranavir, trifluridine, trizivir, tromantadine, truvada, valaciclovir (for example, VALTREX™), valganciclovir, valrubicin, vapreotide, vicriviroc, vidarabine, viramidine, velpatasvir, vivecon, zalcitabine, zanamivir (for example, RELENZA™) or zidovudine; a serine protease inhibitor, optionally camostat; an anti-PD-1 checkpoint inhibitor, optionally camrelizumab; a compound or antibody capable of binding complement factor C5 and blocking membrane attack complex formation, optionally eculizumab; a cathepsin inhibitor, optionally a cathepsin K, B or L inhibitor, optionally relacatib; thalidomide, or thalidomide and glucocorticoid (optionally low-dose glucocorticoid), or and thalidomide and celecoxib; an antibacterial antibiotic or a macrolide drug, wherein optionally the macrolide drug comprises azithromycin (for example, ZITHROMAX™, or AZITHROCIN™) clarithromycin (for example, BIAXIN™), erythromycin (for example, ERYTHROCIN™), or fidaxomicin (for example, DIFICID™ or DIFICLIR™) troleandomycin (for example, TEKMISIN™), tylosin (for example, TYLOCINE™ or TYLAN™), solithromycin (for example, SOLITHERA™), oleandomycin (or SIGMAMYCINE™), midecamycin, roxithromycin, kitasamycin or turimycin, josamycin, carbomycin or magnamycin, and/or spiramycin; opaganib or YELIVA™; an anti-interleukin-6 antibody (e.g., tocilizumab or tocilizumab and favipiravir, for example, ACTEMRA™); sarilumab (for example, KEVZARA™); umifenovir (for example, ARBIDOL™); colchicine, or COLCRYS™, MITIGARE™; a corticosteroid class drug such as budesonide (or RHINOCORT™ or PULMICORT™), prednisolone (or ORAPRED™), methyl-prednisolone, prednisone (or DELTASONE™ or ORASONE™) or hydrocortisone (or CORTEF™); an anti-androgen drug, or bicalutamide; a hydrocortisone or cortisol (or CORTEF™, SOLUCORTEF™), or hydrocortisone sodium succinate or hydrocortisone acetate or dexamethasome (or DEXTENZA™, OZURDEX™, NEOFORDEX™); famotidine, or PEPCID™; an antihistamine class drug such as azelastine, or ASTELIN™, OPTIVAR™ ALLERGODIL™, brompheniramine, fexofenadine or ALLEGRA™, pheniramine or AVIL™, or chlorpheniramine; a dendrimer, or an astodrimer sodium (Starpharma, Melbourne, Australia); a selective serotonin reuptake inhibitor (SSRI) class drug, optionally fluvoxamine, or LUVOX™, FAVERIN™, FLUVOXIN™; a nicotinic antagonist, a dopamine agonist or a noncompetitive N-Methyl-d-aspartic acid or N-Methyl-d-aspartate (NMDA) antagonist; an immunosuppressive drug, or tocilizumab or atlizumab, or ACTEMRA™, or ROACTEMRA™, or a calcineurin inhibitor (CNI), or ciclosporin or cyclosporine or cyclosporin); or, any two, three or more or combination thereof;

and optionally a formulation as provided herein further comprises an antibiotic, or a method as provided herein further comprises administration of an antibiotic, and optionally at least one dose of the antibiotic, for example, a macrolide drug such as azithromycin, is administered before a first administration of the formulation, optionally at least one dose of the antibiotic is administered one day or two days, or more, before a first administration of the formulation;
and optionally a formulation as provided herein further comprises an inhibitor of an inhibitory immune checkpoint molecule, which can comprise a protein or polypeptide that binds to an inhibitory immune checkpoint protein, and optionally the inhibitor of the inhibitory immune checkpoint protein is an antibody or an antigen binding fragment thereof that specifically binds to the inhibitory immune checkpoint protein;
and optionally the inhibitor of the inhibitory immune checkpoint molecule targets a compound or protein comprising: a CTLA4 or CTLA-4 (cytotoxic T-lymphocyte-associated protein 4, also known as CD152, or cluster of differentiation 152); Programmed cell Death protein 1, also known as PD-1 or CD279; Programmed Death-Ligand 1 (PD-L1), also known as cluster of differentiation 274 (CD274) or B7 homolog 1 (B7-H1)); PD-L2; A2AR (adenosine A₂, receptor, also known as ADORA2A); B7-H3; B7-H4; BTLA (B- and T-lymphocyte attenuator protein); KIR (Killer-cell Immunoglobulin-like Receptor); IDO (Indoleamine-pyrrole 2,3-dioxygenase); LAG3 (Lymphocyte-Activation Gene 3 protein); TIM-3; VISTA (V-domain Ig suppressor of T cell activation protein); or any combination thereof;
and optionally the inhibitor of an inhibitory immune checkpoint molecule comprises: ipilimumab or YERVOY®; pembrolizumab or KEYTRUDA®; nivolumab or OPDIVO®; atezolizumab or TECENTRIP®; avelumab or BAVENCIO®; durvalumab or IMFINZI®; AMP-224 (MedImmune), AMP-514 (an anti-programmed cell death 1 (PD-1) monoclonal antibody (mAb) (MedImmune)), PDR001 (a humanized mAb that targets PD-1), STI-A1110 or STI-A1010 (Sorrento Therapeutics), BMS-936559 (Bristol-Myers Squibb), BMS-986016 (Bristol-Myers Squibb), TSR-042 (Tesaro), JNJ-61610588 (Janssen Research & Development), MSB-0020718C, AUR-012, enoblituzumab (also known as MGA271) (MacroGenics, Inc.), MBG453, LAG525 (Novartis), BMS-986015 (Bristol-Myers Squibb), or any combination thereof,
and optionally the inhibitor of the inhibitory immune checkpoint molecule, or the stimulatory immune checkpoint molecule, is administered by: intravenous (IV) injection, intramuscular (IM) injection, intratumoral injection or subcutaneous injection; or, is administered orally or by suppository; or the formulation further comprises at least one immune checkpoint inhibitor;

and optionally the viral infection treated by administration of a combination of microbes or formulations as provided herein, or by practicing a method as provided herein, comprises an infection caused by or associated with: a coronavirus (for example, COVID-19, SARS (Severe Acute Respiratory Syndrome) or MERS (Middle East Respiratory Syndrome))), an influenza virus (for example, influenza A, B or C), adeno-associated virus, aichi virus, coxsackievirus, dengue virus, ebolavirus, an encephalomyocarditis virus, an Epstein-Barr virus, hantaan virus, a hepatitis virus (for example, hepatitis A, B, C, E or delta virus), human respiratory syncytial virus (hRSV), human adenovirus, astrovirus, cytomegalovirus, entervirus, a herpes virus (for example, herpesvirus 1, 2, 6, 7, or 8), human immunodeficiency virus (HIV) (for example, HIV-1), human papillomavirus, parainfluenza virus, parvovirus, human respiratory syncytial virus, a rhinovirus, human spumaretrovirus, human T-lymphotropic virus, torovirus, lymphocytic choriomeningitis virus, measles virus, a polyomavirus (for example, Merkel cell or Wu polyomavirus), mumps virus, Norwalk virus, poliovirus, rabies virus, rosavirus, rotavirus (for example, rotavirus A, B or C), rubella virus, Semliki virus, simian virus, sindbis virus, tick-borne powassan virus, vaccinia virus, varicella-zoster virus, variola virus, equine encephalitis virus, vesicular stomatitis virus, West Nile virus, yellow fever virus or zika virus;

and optionally a method as provided herein is administered with (either before, during or after) administration of an antiviral vaccine, immune enhancer or adjuvant such as for example, NASOVAX™ vaccine by Altimmune, Inc. (Gaithersburg, Md.).

and/or
the method comprises, or further comprises, administering, or having administered, or delivering, a genetically (or recombinantly) engineered cell, wherein optionally the genetically engineered cell is: a microbe or spore derived from a microbe as used in a method of any of the preceding claims, or a method as provided herein; or, a non-pathogenic bacteria or spore form thereof as set forth in Tables 1, 4, 7 or 8; or, a non-pathogenic bacteria or spore form thereof included in a combination of non-pathogenic bacteria and/or spores thereof (or spore derived from) as set forth in Tables 9 and 42,

and optionally the microbe is genetically engineered to express or secrete a heterologous or overexpress an endogenous immunomodulatory molecule, and optionally the immunomodulatory molecule is an immunomodulatory protein or peptide, and optionally the immunomodulatory molecule is an immunostimulatory molecule,

and optionally the microbe is genetically engineered to overexpress a pathway for production of at least one short chain fatty acid (SCFA), and optionally the SCFA comprises butyrate or butyric acid, propionate or acetate,

and optionally the microbe is genetically engineered by inserting a heterologous nucleic acid into the microbe, and optionally the heterologous nucleic acid encodes an exogenous membrane protein,

and optionally the immunostimulatory molecule, protein or peptide comprises a non-specific immunostimulatory protein, and optionally the non-specific immunostimulatory protein comprises a cytokine, and optionally the cytokine comprises an interferon (optionally an IFN-α2a, IFN-α2b), and interleukin (optionally IL-2, IL-4, IL-7, IL-12), an interferon (IFN), a TNF-α, a granulocyte colony-stimulating factor (G-CSF, also known as filgrastim, lenograstim or Neupogen®), a granulocyte monocyte colony-stimulating factor (GM-CSF, also known as molgramostim, sargramostim, Leukomax®, Mielogen® or Leukine®), or any combination thereof,

and optionally the immunostimulatory molecule, protein or peptide comprises a specific immunostimulatory protein or peptide, and optionally the specific immunostimulatory protein or peptide comprises an immunogen that can generate a specific humeral or cellular immune response or an immune response to a viral antigen,

and optionally the genetically engineered cell is a lymphocyte, and optionally the genetically engineered cell expresses a chimeric antigen receptor (CAR), and optionally the lymphocyte is a B cell or a T cell (CAR-T cell), and optionally the lymphocyte is a tumor infiltrating lymphocyte (TIL),

and optionally the microbe is genetically engineered to substantially decrease, reduce or eliminate the microbe's toxicity,

and optionally the microbe is genetically engineered to comprise a kill switch so the microbe can be rendered non-vital after administration of an appropriate trigger or signal,

and optionally the microbe is genetically engineered to secrete anti-inflammatory compositions or have an anti-inflammatory effect,

and optionally the genetically engineered cell is administered or delivered before administration of, simultaneously with, and/or after administration or delivery of the formulation.

In alternative embodiments, provided are formulations or pharmaceutical compositions comprising:

(a) a combination of microbes as set forth in Tables 9 and 42;

(b) a combination of microbes as used in a method as provided herein or as provided herein; and/or

(c) at least two different species or genera (or types) of non-pathogenic bacteria, wherein each of the non-pathogenic bacteria comprise (or are in the form of) a plurality of non-pathogenic colony forming live bacteria, a plurality of non-pathogenic germinable non-pathogenic bacterial spores, or a combination thereof, and the formulation comprises at least one (or any one, several, or all of) non-pathogenic bacteria or spore of the family or genus (or class): Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), or a combination thereof.

In alternative embodiments, of formulations or pharmaceutical compositions as provided herein, or methods as provided herein:
the formulation comprises at least one (or any one, several, or all of) non-pathogenic bacteria or spore form thereof as set forth in Tables 1, 4, 7 or 8, or included in the combination of non-pathogenic bacteria and/or spores thereof (or spore derived from) as set forth in Tables 9 and 42;
the formulation comprises an inner core surrounded by an outer layer of polymeric material enveloping the inner core, wherein the non-pathogenic bacteria or the non-pathogenic germinable bacterial spores are substantially in the inner core, and optionally the polymeric material comprises a natural polymeric material;
the plurality of non-pathogenic colony forming live bacteria are substantially dormant colony forming live bacteria, or the plurality of non-pathogenic colony forming live bacteria or the plurality of non-pathogenic germinable bacterial spores are lyophilized, wherein optionally the non-pathogenic dormant colony forming live bacteria comprise live vegetative bacterial cells that have been rendered dormant by lyophilization or freeze drying;
the formulation comprises at least 1×10⁴ colony forming units (CFUs), or between about 1×10² and 1×10⁸ CFUs, 1×10³ and 1×10⁷ CFUs, or 1×10⁴ and 1×10⁶ CFUs, of live non-pathogenic bacteria and/or non-pathogenic germinable bacterial spores;
the formulation or pharmaceutical composition comprises water, saline, a pharmaceutically acceptable preservative, a carrier, a buffer, a diluent, an adjuvant or a combination thereof;
the formulation or pharmaceutical composition is formulated for administration orally or rectally, or is formulated as a liquid, a food, a gel, a geltab, a candy, a lozenge, a tablet, pill or capsule, or a suppository;
the formulation or pharmaceutical composition further comprises: a biofilm disrupting or dissolving agent, an antibiotic, an inhibitor of an inhibitory immune checkpoint molecule and/or a stimulatory immune checkpoint molecule (or any composition for use in checkpoint blockade immunotherapy);
the inhibitor of an inhibitory immune checkpoint molecule comprises a protein or polypeptide that binds to an inhibitory immune checkpoint protein, and optionally the inhibitor of the inhibitory immune checkpoint molecule is an antibody or an antigen binding fragment thereof that binds to an inhibitory immune checkpoint protein;
the inhibitor of an inhibitory immune checkpoint molecule targets a compound or protein comprising: CTLA4 or CTLA-4 (cytotoxic T-lymphocyte-associated protein 4, also known as CD152, or cluster of differentiation 152); Programmed cell Death protein 1, also known as PD-1 or CD279; Programmed Death-Ligand 1 (PD-L1), also known as cluster of differentiation 274 (CD274) or B7 homolog 1 (B7-H1)); PD-L2; A2AR (adenosine A₂, receptor, also known as ADORA2A); B7-H3; B7-H4; BTLA (B- and T-lymphocyte attenuator protein); KIR (Killer-cell Immunoglobulin-like Receptor); IDO (Indoleamine-pyrrole 2,3-dioxygenase); LAG3 (Lymphocyte-Activation Gene 3 protein); TIM-3; VISTA (V-domain Ig suppressor of T cell activation protein) or any combination thereof;
the inhibitor of an inhibitory immune checkpoint molecule comprises: ipilimumab or YERVOY®; pembrolizumab or KEYTRUDA®; nivolumab or OPDIVO®; atezolizumab or TECENTRIP®; avelumab or BAVENCIO®; durvalumab or IWINZI®; AMP-224 (MedImmune), AMP-514 (an anti-programmed cell death 1 (PD-1) monoclonal antibody (mAb) (MedImmune)), PDR001 (a humanized mAb that targets PD-1), STI-A1110 or STI-A1010 (Sorrento Therapeutics), BMS-936559 (Bristol-Myers Squibb), BMS-986016 (Bristol-Myers Squibb), TSR-042 (Tesaro), JNJ-61610588 (Janssen Research & Development), MSB-0020718C, AUR-012, enoblituzumab (also known as MGA271) (MacroGenics, Inc.), MBG453, LAG525 (Novartis), BMS-986015 (Bristol-Myers Squibb), or any combination thereof, and/or
the stimulatory immune checkpoint molecule comprises a member of the tumor necrosis factor (TNF) receptor superfamily, optionally CD27, CD40, OX40, GITR (a glucocorticoid-Induced TNFR family Related gene protein) or CD137, or comprises a member of the B7-CD28 superfamily, optionally CD28 or Inducible T-cell co-stimulator (ICOS).

In alternative embodiments, provided are kits or products of manufacture comprising a formulation or pharmaceutical composition as provided herein, wherein optionally the product of manufacture is an implant.

In alternative embodiments, provided are uses of a formulation or pharmaceutical composition as provided herein, or a kit or product of manufacture as provided herein, for controlling, ameliorating or treating a cancer in an individual in need thereof.

In alternative embodiments, provided are uses of a formulation or a pharmaceutical composition as provided herein in the manufacture of a medicament for controlling, ameliorating or treating a cancer in an individual in need thereof.

In alternative embodiments, provided are formulations or pharmaceutical compositions as provided herein, or a kit as provided herein, for use in controlling, ameliorating or treating a cancer in an individual in need thereof. In alternative embodiments, the cancer is melanoma, advanced melanoma, cutaneous or intraocular melanoma, primary neuroendocrine carcinoma of the skin, breast cancer, a cancer of the head and neck, uterine cancer, rectal and colorectal cancer, a cancer of the head and neck, cancer of the small intestine, a colon cancer, a cancer of the anal region, a stomach cancer, lung cancer, brain cancer, non-small-cell lung cancer, ovarian cancer, angiosarcoma, bone cancer, osteosarcoma, prostate cancer; cancer of the bladder; cancer of the kidney or ureter or renal cell carcinoma, or carcinoma of the renal pelvis; a neoplasm of the central nervous system (CNS) or renal cell carcinoma.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1 graphically summarizes the classification level of least common ancestors for each cluster. Microbial genome assemblies from NCBI RefSeq are classified into operational species units by clustering similar genome assemblies together. The least common ancestor in the NCBI hierarchy for the assemblies in each operational species unit (OSU) cluster is determined. For OSU's containing more than one microbial assembly, the rank of the least common ancestor is displayed. Most OSU's have a least common ancestor at the species or genus level, demonstrating consistency between the assigned OSU's and the pre-existing NCBI taxonomic tree, as described in Example 9, below.

FIG. 2 graphically shows the distribution of OSU cluster sizes. Microbial genome assemblies from NCBI RefSeq are classified into operational species units by clustering similar genome assemblies together. The cluster size distribution is visualized, as described in Example 9, below.

FIG. 3 graphically illustrates a principal component analysis (PCA) of microbiome composition obtained from fecal samples. Whole genome sequencing is performed on fecal samples from subjects with and without cancer as well as in remission. The reads are classified, and abundance of each operational species unit is estimated computationally. PCA is performed on centered-log-ratio transformed abundances, and the first two principal coordinates are plotted for cancer, remission, and control sample cohorts, as described in Example 9, below.

FIG. 4 graphically illustrates a PCA plot showing the relationship between longitudinal samples of the same patient. Whole genome sequencing is performed on fecal samples from subjects with and without cancer as well as in remission. The reads are classified, and abundance of each operational species unit is estimated computationally. PCA is performed on centered-log-ratio transformed abundances, and the first two principal coordinates are plotted for cancer and control sample cohorts, with longitudinal samples being connected by arrows. Later samples from the same subject are colored darker, as described in Example 9, below.

FIG. 5 graphically illustrates a volcano plot showing the differential abundance of species in cancer and control cohorts. Whole genome sequencing is performed on fecal samples from subjects with and without cancer and the reads are classified and abundance of each operational species unit is estimated computationally. The fold change difference and statistical significance (inverse p value, Mann Whitney U test) is calculated for abundances between cancer and control sample cohorts. The results are displayed on a volcano plot. Each point is an operational species unit, and the area of each point corresponds to the average abundance of that operational species unit across all samples, as described in Example 9, below.

FIG. 6 shows the distribution of abundances of specific organisms among the different patient samples in each cohort. Whole genome sequencing is performed on fecal samples from subjects with and without cancer and the reads are classified and abundance of each operational species unit is estimated computationally. Operational species units with significant differences between cancer and control are displayed, as described in Example 9, below.

FIG. 7 graphically illustrates the distribution of abundances of additional specific organisms among the different patient samples in each cohort, plotted as in FIG. 6.

FIG. 8 graphically illustrates a receiver operating characteristic (ROC) curve of the classifier developed based on stool species distribution. A random forest classifier is trained to classify operational species unit abundances for a sample as corresponding to cancer or control. An ROC curve is generated on 145 cancer samples and 88 control samples using leave-one-out cross validation. No hyperparameter optimization was performed, as described in Example 10, below.

FIG. 9 graphically illustrates correlations of species abundance with immune markers obtained from blood analysis. Immune markers with significant correlations to operational species unit relative abundances are plotted. P values are generated by a linear mixed model fit that model immune marker proportions as being linearly related to the logarithm of OSU abundance, with a random effect accounting for cancer and control groups. For CD3+CD56+, the logarithm of the immune marker proportion is used as the output of the mixed model. (a) positive correlations; (b) negative correlations, as described in Example 10, below.

FIG. 10 graphically illustrates the distribution of abundances of specific organisms in complete responders (CR), partial responders (PR), and non-responders (NR). Whole genome sequencing is performed on the initial time point fecal samples from subjects undergoing cancer immunotherapy and the reads are classified and abundance of each operational species unit is estimated computationally. Operational species unit abundances are correlated to response to therapy using a score of 2 for complete response, 1 for partial response, 0 for no response, using the Spearman rank correlation. Operational species unit abundances for several notable OSUs are displayed with the corresponding Spearman p values, as described in Example 10, below.

FIG. 11 graphically illustrates the first two principal components of a PCA of centered-log-ratio transformed microbial species abundance values obtained from fecal samples of FMT-treated mice 7 days post-treatment. Circles and Xs represent samples from mice treated with fecal material from two different non-responder patients. Squares and plusses represent samples from mice treated with fecal material from two different responder patients. The large symbols of each type indicate species composition of the human fecal material used for each transplant.

FIG. 12 graphically illustrates principal components 2 and 3 of a PCA of centered-log-ratio transformed microbial species abundance values obtained from fecal samples of FMT-treated mice 7 days post-treatment. Symbols are as described for FIG. 11.

FIG. 13 graphically illustrates principal components 3 and 4 of a PCA of centered-log-ratio transformed microbial species abundance values obtained from fecal samples of FMT-treated mice 7 days post-treatment. Symbols are as described for FIG. 11.

FIG. 14 graphically illustrates the first two components of a t-Distributed Stochastic Neighbor Embedding (tSNE) of centered-log-ratio transformed microbial species abundance values obtained from fecal samples of FMT-treated mice 7 days post-treatment. Circles and Xs represent samples from mice treated with fecal material from two different non-responder patients. Squares and plusses represent samples from mice treated with fecal material from two different responder patients.

FIG. 15 graphically illustrates the first two components of a tSNE of centered-log-ratio transformed microbial species abundance values obtained from fecal samples of FMT-treated mice 7, 13, and 27 days post-treatment. Shading intensity of the points indicates different donors. Circles, 7 days post-treatment; Xs, 13 days post-treatment; squares, 27 days post-treatment.

FIG. 16 illustrates Table 2, as discussed in Example 7, below.

FIG. 17 illustrates Table 3, as discussed in Example 9, below.

FIG. 18 illustrates Table 5, as discussed in Example 10, below.

FIG. 19 illustrates Table 6, as discussed in Example 10, below.

FIG. 20 graphically illustrates exemplary flow cytometry analysis of peripheral blood samples from a patient undergoing immunotherapy are shown, as described in Example 9, below.

FIG. 21 graphically illustrates flow cytometry data from immune-phenotyping 73 blood samples obtained from human subjects with and without cancer. Statistical analysis was performed to find significantly different differences in immune markers between cancer and control sample cohorts, using a Mann Whitney U test and filtering for a false discovery rate of 0.05. Markers passing the FDR filter are plotted. The box denotes the 25th, 50th, and 75th percentiles of the data, and each point is a single sample; as discussed in detail in Example 9, below.

FIG. 22 graphically illustrates principal component analysis of flow cytometry data from immune-phenotyping 73 blood samples obtained from human subjects with and without cancer. Principal component analysis is performed on the immune marker percentages and the first two components are plotted by stage of cancer. The P value is computed using permutational multivariate analysis of variance (PERMANOVA); as discussed in detail in Example 9, below.

FIG. 23 graphically illustrates the results of a statistical analysis performed on metabolomics data on plasma obtained from a third-party provider (as “a volcano plot”). A Mann Whitney U test is used to find significantly different metabolites between cancer and control cohorts. Metabolites enriched in cancer samples appear on the right side of the plot and those enriched in control samples occur on the left, with higher points on the y-axis corresponding to increased statistical significance, as discussed in detail in Example 9, below.

FIG. 24 graphically illustrates the results of a principal component analysis comparing immune flow cytometry data to whole genome sequencing data. The primary principal component for the whole genome sequencing data and the second principal component for immune flow cytometry data are plotted against each other, revealing a strong correlation and suggesting that the microbiome may play a role in affecting the immune system and vice versa, as discussed in detail in Example 9, below.

FIG. 25 graphically illustrates the results of a principal component analysis performed on the plasma metabolomics of cancer and control samples, showing clear separation between cancer and control samples, as discussed in detail in Example 9, below.

FIG. 26 graphically illustrates the distribution of Euclidean distances in a centered-log-transformed space between successive longitudinal fecal whole genome sequencing samples for both cancer and control cohorts. The plot shows a higher average distance between longitudinal cancer samples than control, as discussed in detail in Example 9, below.

FIG. 27 graphically illustrates a heatmap of the Spearman correlations calculated between each flow gate (CD11b+, CD3+, CD8-HLADR+ and FoxP3+) for humans and each organism in the gut whose mean abundance is greater than or equal to 0.0005; as discussed in detail in Example 9, below.

FIG. 28 depicts in table form the results of a statistical analysis performed on metabolomics data on plasma obtained from a third-party provider. A Mann Whitney U test is used to find significantly different metabolites between cancer and control cohorts. The top 100 metabolites ranked by p value are reported, as discussed in detail in Example 9, below.

FIG. 29 graphically illustrates data from studies where mice inoculated with CT-26 colon cancer cells were treated with microbial mix 4 and anti-CTLA-4 therapy, the data showing that the anti-CTLA-4 therapy with microbial mix 4 (or “microbe mix 4”) had minimal tumor growth in contrast to the other groups, tumor volume is shown as a function of time since tumor inoculation, as described in Example 15, below.

FIG. 30 illustrates a plot summarizing data from a FACS analysis of whole blood obtained from the animals at the end of a study (as described in Example 15) that indicated that CD4 and CD8 T-lymphocyte activity are increased by treatment with a microbial mix 4 in conjunction with anti-CTLA-4.

FIG. 31 graphically illustrates data from studies where mice inoculated with CT-26 colon cancer cells were treated with microbial mix 2, mix 5 and anti-CTLA4 therapy, the data showing that the anti-CTLA-4 therapy with microbial mix 2 (or “microbe mix 2”) had minimal tumor growth in contrast to the other groups, tumor volume is shown as a function of time since tumor inoculation, as described in Example 15, below.

FIG. 32 graphically illustrates a principal component analysis on metabolome profile from all samples at timepoint T7. Downward cones, Control; circles, Microbe; squares, Drug; and upward cones, Combo; as described in detail in Example 15, below.

FIG. 33 graphically illustrates data of concentrations of pterin and biopterin in mouse samples over time; in order from lightest to darkest lines and symbols, groups are indicated as follows: Control, Microbe, Drug, Combo; as described in detail in Example 15, below.

FIG. 34 graphically illustrates data from studies where mice inoculated with CT-26 colon cancer cells were treated with microbial mix 4 and prebiotic (ellagic acid) therapy, the data showing that the prebiotic therapy (ellagic acid) with microbial mix 4 had minimal tumor growth in contrast to the other groups, tumor volume is shown as a function of time since tumor inoculation, as described in Example 16, below.

FIG. 35 graphically illustrates flow cytometry data from a immune-phenotyping of mice subjected to cancer receiving the different microbial treatments, where measurements were conducted on both peripheral blood and on the tumor itself, with stains for various cell surface markers, where final tumor volume is a function of CD3+ proportion in CD45 cells (left image) or CD4 to CD8 ratio in CD3+ cells (right image), as discussed in detail in Example 16, below.

FIG. 36 graphically illustrates Spearman correlations between immune cell populations and final tumor volume for all treatment groups and magnitude is plotted by GI location (small intestine, cecum and colon); as discussed in detail in Example 17, below.

FIG. 37 graphically illustrates the statistically significant correlation between final tumor volume for all treatment groups and the IA/IE (MHC II) immune cell populations in the colon for all treatment groups; as discussed in detail in Example 17, below.

FIG. 38 graphically illustrates flow cytometry data on mice 22 days post-inoculation and CD3+ percentage is displayed against tumor volume at day 28 post-inoculation; as discussed in detail in Example 17, below.

FIG. 39 graphically illustrates tumor volumes for mice remaining alive (10 mice initially per group) 28 days post tumor inoculation; as discussed in detail in Example 17, below.

FIG. 40 graphically illustrates tumor volumes over time for mice treated with anti-PD1 alone or in conjunction with microbial mix 2; as discussed in detail in Example 17, below.

FIG. 41 graphically illustrates tumor volumes that were measured 28 days post inoculation and displayed by both pre-treatment and treatment groups; as discussed in detail in Example 17, below.

FIG. 42A-B graphically illustrates tumor volumes that were measured at multiple time points post-inoculation. Mean and standard error of the mean are displayed for each treatment group within water (FIG. 42A) and antibiotic (FIG. 42B) pre-treatment groups; as discussed in detail in Example, 22 below.

FIG. 43 graphically illustrates tumor volume distribution with and without microbial mix 2 being administered for each FMT donor. The box denotes the 25th, 50th, and 75th percentiles of the data, and each point is a single mouse; as discussed in detail in Example 17, below.

FIG. 44 graphically illustrates the mean tumor volume over time for mice receiving microbial mix 2 vs Vehicle for each fecal transplant donor. Error bars are standard error of the mean; as discussed in detail in Example 17, below.

FIG. 45 graphically illustrates the mean tumor volume over time for mice receiving microbial mix 2 vs Vehicle for each fecal transplant donor. Each dot denotes an individual mouse's tumor volume; as discussed in detail in Example, 17 below.

FIG. 46A-D graphically illustrates images of the gastrointestinal tract at day 21 for mice pre-treated with either water or antibiotics and treatments including vehicle, anti-CTLA-4, anti-CTLA-4 in combination with microbial mix 4+ ellagic acid and anti-CTLA-4 in combination with microbial mix 2; as discussed in detail in Example 17, below.

FIG. 47 illustrates Table 33, as discussed in Example 24, below.

FIG. 48 graphically illustrates the CyTOF gating strategy used to classify immune cell populations based on metal-labeled peptide markers, and cell counts for a representative sample; as discussed in detail in Example 24, below.

FIG. 49 illustrates Table 40, as discussed in Example 25, below.

FIG. 50 illustrates Table 41, as discussed in Example 25, below.

FIG. 51 graphically represents survival of mice following FMT treatment and challenge with influenza A/CA/04/2009 (H1N1pdm) virus; Kaplan-Meier survival curves were generated and compared by the Log-rank (Mantel-Cox) test followed by pairwise comparison using the Gehan-Breslow-Wilcoxon test in PRISM 9.0.2™ (GraphPad Software Inc.); mean body weights were analyzed by one-way analysis of variance (ANOVA) followed by Tukey's multiple comparison tests using PRISM™; the mean day of death was calculated using PRISM™ and differences in mean day of death were evaluated by one-way ANOVA; no significant difference in survival post-infection was observed between infected HC-FMT or NR-FMT treated mice.

FIG. 52 graphically illustrates mean body weights of mice following FMT treatment, then challenge with influenza A/CA/04/2009 (H1N1pdm) virus.

FIG. 53 is a box plot showing lung virus titers of mice following FMT treatment and challenge with influenza A/CA/04/2009 (H1N1pdm) virus.

FIG. 54 illustrates lung scores of visible pathogenicity of mice following FMT treatment and challenge with influenza A/CA/04/2009 (H1N1pdm) virus.

FIG. 55 graphically illustrates lung weights of mice following FMT treatment and challenge with influenza A/CA/04/2009 (H1N1pdm) virus.

FIG. 56 graphically illustrates lung cytokine concentrations of IL-1a, IL-1b, IL-2, and IL-3 of mice treated with HC- or NR-FMT, before (uninfected) and following challenge with influenza A/CA/04/2009 (H1N1pdm) virus (infected). Welch's t-test was applied to identify those with significant differential abundance between and non-responders FMT HC-FMT and NR-FMT recipients (N=9 for each group). P-values are shown for groups in cases where they are less than 0.05.

FIG. 57 graphically illustrates lung cytokine concentrations of IL-4, IL-5, IL-6, and IL-10 of mice treated with HC- or NR-FMT, before (uninfected) and following challenge with influenza A/CA/04/2009 (H1N1pdm) virus (infected). Welch's t-test was applied to identify those with significant differential abundance between and non-responders FMT HC-FMT and NR-FMT recipients (N=9 for each group). P-values are shown for groups in cases where they are less than 0.05.

FIG. 58 graphically illustrates lung cytokine concentrations of IL-12p70, IL-17, MCP-1, and IFN-γ of mice treated with HC- or NR-FMT, before (uninfected) and following challenge with influenza A/CA/04/2009 (H1N1pdm) virus (infected). Welch's t-test was applied to identify those with significant differential abundance between and non-responders FMT HC-FMT and NR-FMT recipients (N=9 for each group). P-values are shown for groups in cases where they are less than 0.05.

FIG. 59 graphically illustrates lung cytokine concentrations of TNFα, MIP-1α, GM-CSF, and RANTES of mice treated with HC- or NR-FMT, before (uninfected) and following challenge with influenza A/CA/04/2009 (H1N1pdm) virus (infected); Welch's t-test was applied to identify those with significant differential abundance between and non-responders FMT HC-FMT and NR-FMT recipients (N=9 for each group); P-values are shown for groups in cases where they are less than 0.05.

FIG. 60 presents metagenomic analyses of FMT preparations made from feces from the HC and NR human donors; each slice of the donut plot indicates the fractional abundance of a microbial taxon, in most case a species or strain, in the complete metagenome; taxa that fall below 1.0% total abundance are grouped together as “Other”.

Like reference symbols in the various drawings indicate like elements.

### DETAILED DESCRIPTION

In alternative embodiments, provided are compositions, including products of manufacture and kits, and methods, comprising novel combinations of microbes, also called live biotherapeutic compositions such as non-pathogenic, live (optionally dormant) bacteria and/or bacterial spores, for example, such as the exemplary combinations of microbes as listed in Table 9, Example 10 and Table 42, Example 25. In alternative embodiments, the compositions, products of manufacture, kits and methods as provided herein are used as a therapy (for example, as a mono-therapy or as a co-therapy, or co-treatment) for the control, amelioration and/or treatment of a disease or condition, for example, a viral infection such as a coronavirus infection. In alternative embodiments, the compositions, products of manufacture, kits and/or methods as provided herein are administered to an individual receiving a drug, for example, an anti-viral therapy, thereby resulting in a modification or modulation of the patient's gut microfloral population(s), thus resulting in an enhancement of the drug therapy, for example, lowering the dosage or amount of drug needed for effective therapy, or the frequency with which a drug must be administered to be effective. In alternative embodiments, by modulating or modifying the individual's gut microbial population(s) using compositions, products of manufacture and methods as provided herein, the pharmacodynamics of a drug administered to the patient is altered, for example, the pharmacodynamics of the drug is enhanced, for example, the individual's ability to absorb a drug is modified (for example, accelerated or slowed, or enhanced), or the dose efficacy of a drug is increased (for example, resulting in needing a lower dose of drug for an intended effect), or the gut microbes act orthogonally on the drug target (for example, resulting in the presence of the microbe being essential for the drug to have the intended effect). For example, in alternative embodiments, by modulating or modifying the patient's gut microbial population(s) using compositions, products of manufacture and methods as provided herein the dose efficacy of a drug, for example, an anti-viral drug, vaccine, or therapy, is increased, thereby enhancing the control, amelioration or treatment of that viral infection.

In alternative embodiments, the amount, identity, presence, and/or ratio of gut microbiota in a subject is manipulated to facilitate a mono-therapy or one or more co-treatments; for example, in alternative embodiments, combinations of microbes as provided herein are administered with (for example, concurrent with, or before and/or after) an anti-viral treatment.

Described here for the first time are novel combinations of specific microbes, for example, bacteria, including for example microbes found in a human gut or recombinantly engineered or cultured microbes, which can be administered as a mono-therapy or as a co-therapy for, in alternative embodiments, patients having or suspected of having or at increased risk of having a viral infection such as a coronavirus infection, where in alternative embodiments the patients are already undergoing an immune checkpoint inhibitor treatment, or are already undergoing a chemotherapy, a radiation therapy, an immune checkpoint inhibitor, a Chimeric Antigen Receptor (CAR) T-cell therapy (CAR-T) or other immunotherapy or cancer treatment.

In alternative embodiments, provided are therapeutic compositions, including formulations and pharmaceutical compositions, comprising non-pathogenic (optionally dormant) live microbes such as bacteria and/or germination-competent bacterial spores, which can be used for the prevention, amelioration or treatment of a viral infection or the side effects of an anti-viral therapy, for example, a drug therapy or anti-viral vaccine, or can be used or administered before, with or after a chemotherapy, a radiation therapy, an immune checkpoint inhibitor, a Chimeric Antigen Receptor (CAR) T-cell therapy (CAR-T) or other immunotherapy or cancer treatment.

In alternative embodiments, therapeutic compositions, formulations or pharmaceutical compositions as provided herein, or used to practice methods as provided herein, comprise colony forming (optionally dormant) live bacteria and/or germinable bacterial spores which can be used in mono- or co-therapies, for example, as an adjuvant to an antineoplastic treatment administered to a cancer patient, or administered with or as a supplement to a chemotherapy, a radiation therapy, an immune checkpoint inhibitor, a Chimeric Antigen Receptor (CAR) T-cell therapy (CAR-T) or other immunotherapy or cancer treatment.

In some embodiments, a therapeutic composition as provided herein acts or is used as a probiotic composition which can be administered with, before and/or after a chemotherapy, a radiation therapy, an immune checkpoint inhibitor, a Chimeric Antigen Receptor (CAR) T-cell therapy (CAR-T) or other immunotherapy or cancer treatment. In alternative embodiments, therapeutic compositions (for example, the formulations) as provided herein, comprise the bacteria and/or spores and an antineoplastic active agent such as an immune checkpoint inhibitor.

In alternative embodiments, therapeutic compositions, formulations or pharmaceutical compositions as provided herein, or used to practice methods as provided herein, comprise colony forming (optionally dormant) live bacteria and/or germinable bacterial spores for use as a mono-therapy or in combination with (for example, as a co-therapy) or supplementary to a drug (which can be a small molecule or a protein, for example, a therapeutic antibody) blocking an immune checkpoint for inducing immuno-stimulation in a cancer patient. The therapeutic composition as provided herein and the drug (for example, an antibody) can be administered separately or together, or at different time points or at the same time, or can be administered sequentially or concurrently.

In alternative embodiments, therapeutic compositions, formulations or pharmaceutical compositions as provided herein comprise colony forming (optionally dormant) live bacteria and/or germinable bacterial spores which can be used as an adjuvant to an anti-cancer or antineoplastic treatment, for example, an immune checkpoint treatment, administered to a cancer patient. In alternative embodiments, the therapeutic composition comprises the antineoplastic or immune checkpoint active agents. In alternative embodiments, the therapeutic composition, formulations or pharmaceutical compositions as provided herein are administered with or after, or both with and after, administration of the antineoplastic or immune checkpoint active agent.

In alternative embodiments, the formulation or pharmaceutical composition further comprises, or is manufactured with, an outer layer of polymeric material (for example, natural polymeric material) enveloping, or surrounding, a core that comprises the combination of microbes as provided herein.

In alternative embodiments, therapeutic compositions, formulations or pharmaceutical compositions as provided herein, or used to practice methods as provided herein, can comprise a pharmaceutically acceptable carrier, diluent, and/or adjuvant. In other embodiments a pharmaceutically acceptable preservative is present. In yet other embodiments, a pharmaceutically acceptable germinate is present. In still other embodiments the therapeutic composition contains, or further comprises, a nutrient such as inulin, beta-glucan, mannitol, mucin, L-tryptophan, tryptamine, 5-hydroxytryptophan, or niacin, or an immunostimulant such as polyinosinic-polycytidylic acid (poly I.C) at an effective dose of 0.005, 0.05, 0.5, 5.0 milligrams per kilogram body weight

In alternative embodiments, therapeutic compositions, formulations or pharmaceutical compositions as provided herein, or used to practice methods as provided herein, are in the form of a tablet, geltab or capsule, for example, a polymer capsule such as a gelatin or a hydroxypropyl methylcellulose (HPMC, or hypromellose) capsule (for example, VCAPS PLUS™ (Capsugel, Lonza)). In other embodiments, the therapeutic compositions, formulations or pharmaceutical compositions are in or are manufactured as a food or drink, for example, an ice, candy, lolly or lozenge, or any liquid, for example, in a beverage.

In alternative embodiments, therapeutic compositions, formulations or pharmaceutical compositions as provided herein, or used to practice methods as provided herein, comprise at least one bacterial type that is not detectable, of low natural abundance, or not naturally found, in a healthy or normal subject's (for example, human) gastrointestinal tract. In alternative embodiments, the gastrointestinal tract refers to the stomach, the small intestine, the large intestine and the rectum, or combinations thereof.

In alternative embodiments, provided are methods of ameliorating or treating cancer and/or at least one symptom resulting from a cancer therapy or of a condition of the gastrointestinal tract.

In alternative embodiments, by administration of a therapeutic composition, formulation or pharmaceutical composition as provided herein to a subject, or practicing a method as provided herein, the microbiome population or composition of the subject is modulated or altered.

In alternative embodiments, the term “microbiome” encompasses the communities of microbes that can live sustainably and/or transiently in and on a subject's body, for example, in the gut of a human, including bacteria, viruses and bacterial viruses, archaea, and eukaryotes. In alternative embodiments, the term “microbiome” encompasses the “genetic content” of those communities of microbes, which includes the genomic DNA, RNA (ribosomal-, messenger-, and transfer-RNA), the epigenome, plasmids, and all other types of genetic information.

In alternative embodiments, the term “subject” refers to any animal subject including humans, laboratory animals (for example, primates, rats, mice), livestock (for example, cows, sheep, goats, pigs, turkeys, and chickens), and household pets (for example, dogs, cats, and rodents). The subject may be suffering from a disease, for example, a cancer.

In alternative embodiments, the term “type” or “types” when used in conjunction with “bacteria” or “bacterial” refers to bacteria differentiated at the genus level, the species level, the sub-species level, the strain level, or by any other taxonomic method known in the art.

In alternative embodiments, the phrase “dormant live bacteria” refers to live vegetative bacterial cells that have been rendered dormant by lyophilization or freeze drying. Such dormant live vegetative bacterial cells are capable of resuming growth and reproduction immediately upon resuscitation.

In alternative embodiments, the term “spore” also includes “endospore”, and these terms can refer to any bacterial entity which is in a dormant, non-vegetative and non-reproductive stage, including spores that are resistant to environmental stress such as desiccation, temperature variation, nutrient deprivation, radiation, and chemical disinfectants. In alternative embodiments, “spore germination” refers to the dormant spore beginning active metabolism and developing into a fully functional vegetative bacterial cell capable of reproduction and colony formation. In alternative embodiments, “germinant” is a material, composition, and/or physical-chemical process capable of inducing vegetative growth of a dormant bacterial spore in a host organism or in vitro, either directly or indirectly.

In alternative embodiments, the term “colony forming” refers to a vegetative bacterium that is capable of forming a colony of viable bacteria or a spore that is capable of germinating and forming a colony of viable bacteria.

In alternative embodiments, the term “natural polymeric material” comprises a naturally occurring polymer that is not easily digestible by human enzymes so that it passes through most of the human digestive system essentially intact until it reaches the large or small intestine.

In alternative embodiments, therapeutic compositions, formulations or pharmaceutical compositions as provided herein comprise population(s) of non-pathogenic dormant live bacteria and/or bacterial spores. The dormant live bacteria can be capable of colony formation and, in the case of spores, germination and colony formation. Thus, in alternative embodiments, compositions are useful for altering a subject's gastrointestinal biome, for example, by increasing the population of those bacterial types or microorganisms, or are capable of altering the microenvironment of the gastrointestinal biome, for example, by changing the chemical microenvironment or disrupting or degrading intestinal mucin or biofilm, thereby providing treatment of cancer, gastrointestinal conditions, and symptoms resulting from cancer therapy, ultimately increasing the health of the subject to whom they are administered.

In alternative embodiments, the terms “purify,” purified,” and “purifying” are used interchangeably to describe a population's known or unknown composition of bacterial type(s), amount of that bacterial type(s), and/or concentration of the bacterial type(s); a purified population does not have any undesired attributes or activities, or if any are present, they can be below an acceptable amount or level. In alternative embodiments, the various populations of bacterial types are purified, and the terms “purified,” “purify,” and “purifying” refer to a population of desired bacteria and/or bacterial spores that have undergone at least one process of purification; for example, a process comprising screening of individual colonies derived from fecal matter for a desired phenotype, such as their effectiveness in enhancing the pharmacodynamics of a drug (such as a cancer drug, for example, a drug inhibitory to an immune checkpoint), for example, the individual's ability to absorb a drug is modified (for example, accelerated or slowed, or enhanced), or the dose efficacy of a drug is increased (for example, resulting in needing a lower dose of drug for an intended effect), or the immune system is primed for improved drug efficacy, or a selection or enrichment of the desired bacterial types.

Enrichment can be accomplished by increasing the amount and/or concentration of the bacterial types, such as by culturing in a media that selectively favors the growth of certain types of microbes, by screening pure microbial isolates for the desired genotype, or by a removal or reduction in unwanted bacterial types.

In alternative embodiments, bacteria used to practice compositions and methods provided herein are derived from fecal material donors that are in good health, have microbial biomes associated with good health, and are typically free from antibiotic administration during the collection period and for a period of time prior to the collection period such that no antibiotic remains in the donor's system. In alternative embodiments, the donor subjects do not suffer from and have no family history of renal cancer, bladder cancer, breast cancer, prostate cancer, lymphoma, leukemia, autoimmune disease. In alternative embodiments, donor subjects are free from irritable bowel disease, irritable bowel syndrome, celiac disease, Crohn's disease, colorectal cancer, anal cancer, stomach cancer, sarcomas, any other type of cancer, or a family history of these diseases. In alternative embodiments, donor subjects do not have and have no family history of mental illness, such as anxiety disorder, depression, bipolar disorder, autism spectrum disorders, panic disorders, obsessive-compulsive disorder, attention-deficit disorders, eating disorders (for example bulimia, anorexia), mood disorder or schizophrenia. In yet other embodiments the donor subjects have no knowledge or history of food allergies or sensitivities.

In alternative embodiments, the health of fecal matter donors is screened prior to the collection of fecal matter, such as at 1, 2, 3, 4, 8, 16, 20, 24, 28, 32, 36, 40, 44, 48, or 52 weeks pre-collection. In alternative embodiments, fecal matter donors are also screened post-collection, such as at 1, 2, 3, 4, 8, 16, 20, 24, 28, 32, 36, 40, 44, 48, or 52 weeks post-collection. Pre- and post-screening can be conducted daily, weekly, bi-weekly, monthly, or yearly. In alternative embodiments, individuals who do not test positive for pathogenic bacteria and/or viruses (for example, coronavirus, HIV, hepatitis, polio, adeno-associated virus, pox, coxsackievirus, etc.) pre- and post-collection are considered verified donors.

In alternative embodiments, to purify bacteria and/or bacterial spores, fecal matter is collected from donor subjects and placed in an anaerobic chamber within a short time after elimination, such as no more than 1 minute, 5 minutes, 10 minutes, 15 minutes, 20 minutes, 25 minutes, 30 minutes, 35 minutes, 40 minutes, 45 minutes, 50 minutes, 55 minutes, or 60 minutes or more after elimination. In alternative embodiments, fecal matter is collected from donor subjects are placed in an anaerobic chamber within between about 1 minute and 48 hours, or more, after elimination from the donor.

Bacteria from a sample of the collected fecal matter can be collected in several ways. For example, the sample can be mixed with anoxic nutrient broth, dilutions of the resulting mixture conducted, and bacteria present in the dilutions grown on solid anoxic media. Alternatively, bacteria can be isolated by streaking a sample of the collected material directly on anoxic solid media for growth of isolated colonies. In alternative embodiments, to increase the ease of isolating bacteria from fecal samples mixed with anoxic nutrient broth, the resulting mixture can be shaken, vortexed, blended, filtered, and centrifuged to break up and/or remove large non-bacterial matter.

In alternative embodiments, purification of the isolated bacteria and/or bacterial spores by any means known in the art, for example, contamination by undesirable bacterial types, host cells, and/or elements from the host microbial environment can be eliminated by reiterative streaking to single colonies on solid media until at least two replicate streaks from serial single colonies show only a single colony morphology. Purification can also be accomplished by reiterative serial dilutions to obtain a single cell, for example, by conducting multiple 10-fold serial dilutions to achieve an ultimate dilution of 10⁻², 10⁻³, 10⁻⁴, 10⁻⁵, 10⁻⁶, 10⁻⁷, 10⁻⁸, 10⁻⁹ or greater. Any methods known to those of skill in the art can also be applied.

Confirmation of the presence of only a single bacterial type can be confirmed in multiple ways such as, gram staining, PCR, DNA sequencing, enzymatic analysis, metabolic profiling/analysis, antigen analysis, and flow cytometry using appropriate distinguishing reagents.

In alternative embodiments, purified population(s) of vegetative bacteria that are incorporated into therapeutic bacterial compositions as provided herein, or used to practice methods as provided herein, are fermented in growth media. Suitable growth media include Nutrient Broth (Thermo Scientific™ Oxoid™), Anaerobe Basal Broth (Thermo Scientific™ Oxoid™), Reinforced Clostridial Medium (Thermo Scientific™ Oxoid™), Schaedler Anaerobic Broth (Thermo Scientific™ Oxoid™), MRS Broth (Millipore-Sigma™), Vegitone Actinomyces Broth (Millipore-Sigma™), Vegitone Infusion Broth (Millipore-Sigma™), Vegitone Casein Soya Broth (Millipore-Sigma™), or one of the following media available from Anaerobe Systems: Brain Heart Infusion Broth (BHI), Campylobacter-Thioglycollate Broth (CAMPY-THIO), Chopped Meat Broth (CM), Chopped Meat Carbohydrate Broth (CMC), Chopped Meat Glucose Broth (CMG), Cycloserine Cefoxitin Mannitol Broth with Taurocholate Lysozyme Cysteine (CCMB-TAL), Oral Treponeme Enrichment Broth (OTEB), MTGE-Anaerobic Enrichment Broth (MTGE), Thioglycollate Broth with Hemin, Vit. K, without indicator, (THIO), Thioglycollate Broth with Hemin, Vit. K, without indicator, (THIO), Lactobacilli-MRS Broth (LMRS), Brucella Broth (BRU-BROTH), Peptone Yeast Extract Broth (PY), PY Glucose (PYG), PY Arabinose, PY Adonitol, PY Arginine, PY Amygdalin, PYG Bile, PY Cellobiose, PY DL-Threonine, PY Dulcitol, PY Erythritol, PY Esculin, PYG Formate/Fumarate for FA/GLCf, PY Fructose, PY Galactose, PYG Gelatin, PY Glycerol, Indole-Nitrate Broth, PY Inositol, PY Inulin, PY Lactate for FA/GLCf, PY Lactose, PY Maltose, PY Mannitol, PY Mannose, PY Melezitose, PY Melibiose, PY Pyruvic Acid, PY Raffinose, PY Rhamnose, PY Ribose, PY Salicin, PY Sorbitol, PY Starch, PY Sucrose, PY Trehalose, PY Xylan, PY Xylose, Reinforced Clostridial Broth (RCB), Yeast Casitone Fatty Acids Broth with Carbohydrates (YCFAC Broth). In alternative embodiments, growth media includes or is supplemented with reducing agents such as L-cysteine, dithiothreitol, sodium thioglycolate, and sodium sulfide. In alternative embodiments, fermentation is conducted in stirred-tank fermentation vessels, performed in either batch or fed-batch mode, with nitrogen sparging to maintain anaerobic conditions. pH is controlled by the addition of concentrated base, such as NH₄OH or NaOH. In the case of fed-batch mode, the feed is a primary carbon source for growth of the microorganisms, such as glucose. In alternative embodiments, the post-fermentation broth is collected, and/or the bacteria isolated by ultrafiltration or centrifugation and lyophilized or freeze dried prior to formulation.

In alternative embodiments, purified and isolated vegetative bacterial cells used in therapeutic bacterial compositions as provided herein, or used to practice methods as provided herein, have been made dormant; noting that bacterial spores are already in a dormancy state. Dormancy of the vegetative bacterial cells can be accomplished by, for example, incubating and maintaining the bacteria at temperatures of less than 4° C., freezing and/or lyophilization of the bacteria. Lyophilization can be accomplished according to normal bacterial freeze-drying procedures as used by those of skill in the art, such as those reported by the American Type Culture Collection (ATCC) on the ATCC website (see, for example, (https://www.atcc.org).

In alternative embodiments, the purified population of dormant live bacteria and/or bacterial spores has undetectable levels of pathogenic activities, such as the ability to cause infection and/or inflammation, toxicity, an autoimmune response, an undesirable metabolic response (for example diarrhea), or a neurological response.

In alternative embodiments, all of the types of dormant live bacteria or bacterial spores present in a purified population are obtained from fecal material treated as described herein or as otherwise known to those of skill in the art. In other embodiments, one or more of the types of dormant live bacteria or bacterial spores present in a purified population is generated individually in culture and combined with one or more types obtained from fecal material. In alternative embodiments, all of the types of dormant live bacteria or bacterial spores present in a purified population are generated individually in culture. In still other embodiments, one or all of the types of dormant live bacteria and/or bacterial spores present in a purified population are non-naturally occurring or engineered. In yet other embodiments, non-naturally occurring or engineered non-bacterial microorganisms are present, with or without dormant live bacteria and/or bacterial spores.

In alternative embodiments, bacterial compositions used in compositions as provided herein, or to practice methods as provided herein, comprise combinations of different bacteria, for example, comprising at least 2, 3, 4, 5, 6, 7, 8, 9, 10 or more bacterial types, or more than 20 bacterial types, or between about 2 and 30 bacterial types.

In alternative embodiments, the bacterial compositions comprise at least about 10², 10³, 10⁴, 10⁵, 10⁶, 10⁷, 10⁸, 10⁹, 10¹⁰, 10¹¹, 10¹², 10¹³, 10¹⁴, or more (or between about 10² to 10¹⁵) microbes, for example, dormant live bacteria and/or bacterial spores. In some embodiments each bacterial type is equally represented in the total number of dormant live bacteria and/or bacterial spores. In other embodiments, at least one bacterial type is represented in a higher amount than the other bacterial type(s) found in the composition.

In alternative embodiments, a population of different bacterial types used in compositions as provided herein, or to practice methods as provided herein, can increase microbe populations found in the subject's gastrointestinal tract by at least about 10%, 20%, 30%, 40%, 50%, 60%, 70%, 80%, 90%, 100%, 200%, 300%, 400%, 500%, 600%, 700%, 800%, 900% or 1000%, or between about 5% and 2000%, as compared to the subject's microbiome gastrointestinal population prior to treatment.

In alternative embodiments, the combination of microbes, for example, combination of bacterial cells and/or spores, used in compositions as provided herein, or to practice methods as provided herein, are mixed with pharmaceutically acceptable excipients, such as diluents, carriers, adjuvants, binders, fillers, salts, lubricants, glidants, disintegrants, coatings, coloring agents, etc. Examples of such excipients are acacia, alginate, alginic acid, aluminum acetate, benzyl alcohol, butyl paraben, butylated hydroxy toluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium stearate, calcium disodium EDTA, copolyvidone, calcium hydrogen phosphate dihydrate, cetylpyridine chloride, cysteine HCL, crossprovidone, calcium phosphate di or tri basic, dibasic calcium phosphate, disodium hydrogen phosphate, dimethicone, erythrosine sodium, ethyl cellulose, gelatin, glyceryl monooleate, glycerin, glycine, glyceryl monostearate, glyceryl behenate, hydroxy propyl cellulose, hydroxyl propyl methyl cellulose, hypromellose, HPMC phthalate, iron oxides or ferric oxide, iron oxide yellow, iron oxide red or ferric oxide, lactose hydrous or anhydrous or monohydrate or spray dried, magnesium stearate, microcrystalline cellulose, mannitol, methyl cellulose, magnesium carbonate, mineral oil, methacrylic acid copolymer, magnesium oxide, methyl paraben, providone or PVP, PEG, polysorbate 80, propylene glycol, polyethylene oxide, propylene paraben, polaxamer 407 or 188, potassium bicarbonate, potassium sorbate, potato starch, phosphoric acid, polyoxy140 stearate, sodium starch glycolate, starch pregelatinized, sodium carmellose, sodium lauryl sulfate, starch, silicon dioxide, sodium benzoate, stearic acid, sucrose, sorbic acid, sodium carbonate, saccharin sodium, sodium alginate, silica gel, sorbiton monooleate, sodium stearyl fumarate, sodium chloride, sodium metabisulfite, sodium citrate dihydrate, sodium starch, sodium carboxy methyl cellulose, succinic acid, sodium propionate, titanium dioxide, talc, triacetin, and triethyl citrate.

In alternative embodiments, the combinations of microbes, for example, combination of bacterial cells and/or spores, used in compositions as provided herein, or to practice methods as provided herein, are fabricated as colonic or microflora-triggered delivery systems, as described for example, in Basit et al, J. Drug Targeting, 17:1, 64-71; Kotla, Int J Nanomedicine. 2016; 11: 1089-1095; Bansai et al, Polim Med. 2014 April-June; 44(2):109-18; or, Shah et al, Expert Opin Drug Deliv. 2011 June; 8(6):779-96.

In alternative embodiments, combinations of microbes, for example, combination of bacterial cells and/or spores, used in compositions as provided herein, or to practice methods as provided herein, are encapsulated in at least one polymeric material, for example, a natural polymeric material, such that there is a core of bacterial cells and/or spores surrounded by a layer of the polymeric material, for example, a polysaccharide. Examples of suitable polymeric materials are those that have been demonstrated to remain intact through the GI tract until reaching the small or large intestine, where they are degraded by microbial enzymes in the intestines. Exemplary natural polymeric materials can include, but are not restricted to, chitosan, inulin, guar gum, xanthan gum, amylose, alginates, dextran, pectin, khava, and albizia gum (Dafe et al. (2017) Int J Biol Macromol; Kofla et al. (2016) Int J Nanomedicine 11:1089-1095).

In alternative embodiments, compositions provided herein are suitable for therapeutic administration to a human or other mammal in need thereof. In alternative embodiments the compositions are produced by a process comprising, for example: (a) obtaining fecal material from a mammalian donor subject, (b) subjecting the fecal material to at least one purification treatment under conditions that produce a single bacterial type population of bacteria and/or bacterial spores, or a combination of bacterial types and/or bacterial spores, (c) optionally combining the purified population with another purified population obtained from the same or different fecal material, from cultured conditions, or from a genetic stock center such as ATCC or DSMZ, (d) if the microbes, for example, bacterial cells, are not dormant, then treating the purified population(s) under conditions that cause vegetative bacterial cells to become dormant, and (e) placing the dormant bacteria and/or bacterial spores in a vehicle for administration.

In alternative embodiments, formulations and pharmaceutical compositions, and microbes, for example, bacterial cells and/or spores, used in compositions as provided herein or to practice methods as provided herein, are formulated for oral or gastric administration to a mammalian subject. In particular embodiments, the composition is formulated for oral administration as a solid, semi-solid, gel or liquid form, such as in the form of a pill, tablet, capsule, lozenge, food, extract or beverage. Examples of suitable foods are those that require little mastication, such as yogurt, puddings, gelatins, and ice cream. Examples of extracts include crude and processed pomegranate juice, strawberry, raspberry and blackberry. Examples of suitable beverages include cold beverages, such as juices (pomegranate, raspberry, blackberry, blueberry, cranberry, acai, cloudberry, etc., and combinations thereof) and teas (green, black, etc.) and oaked wine.

In alternative embodiments, formulations and pharmaceutical compositions further comprise, or methods as provided herein further comprise administration of, at least one antibiotic (including anti-bacterials or anti-virals), for example, a tetracycline class drug such as doxycycline, chlortetracycline, tetracycline hydrochloride, oxytetracycline, demeclocycline, methacycline or minocycline, penicillin, amoxycillin, erythromycin, vancomycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, ciprofloxacin, sparfloxacin, levofloxacin, rifabutin, rifampicin, rifapentin, sulfisoxazole, sulfamethoxazole, sulfadiazine, sulfadoxine, sulfasalazine, sulfaphenazole, dapsone, sulfacytidine, linezolid or any combination thereof. In alternative embodiments, the antibiotic or a combination of antibiotics are administered before, during and/or after administration of formulations and pharmaceutical compositions as provided herein.

### Gradual or Delayed Release Formulations

In alternative embodiments, exemplary formulations comprise, contain or are coated by an enteric coating to protect a microbe, for example, a bacteria, in a formulation and pharmaceutical compositions as provided herein to allow it to pass through the stomach and small intestine (for example, protect the administered combination of microbes such that a substantial majority of the microbes remain viable), although spores are typically resistant to the stomach and small intestines.

In alternative embodiments, compositions and formulations as provided herein, and compositions and formulations used to practice methods as provided herein, are formulated with a delayed release composition or formulation, coating or encapsulation. In alternative embodiments, compositions and formulations as provided herein, and compositions and formulations used to practice methods as provided herein, are designed or formulated for implantation of living microbes, for example, bacteria or spores, into the gut, including the intestine and/or the distal small bowel and/or the colon. In this embodiment the living microbes, for example, bacteria pass the areas of danger, for example, stomach acid and pancreatic enzymes and bile, and reach the intestine substantially undamaged to be viable and implanted in the GI tract.

In alternative embodiments, a formulation or pharmaceutical preparation, or the combination of microbes contained therein, is liquid, frozen or freeze-dried. In alternative embodiments, for example, for an encapsulated formulation, all are in powdered form. In alternative embodiments, if a formulation or pharmaceutical preparation as provided herein is in a powdered, lyophilate or freeze-dried form, the powder, lyophilate or freeze-dried form can be in a container such as a bottle, cartridge, packet or packette, or sachet, and the powder, lyophilate or freeze-dried form can be hydrated or reconstituted by a liquid, for example by adding water, saline, juice, milk and the like to the powder, lyophilate or freeze-dried form, for example, the powdered, lyophilate or freeze-dried form can be added to the liquid. In alternative embodiments, a powdered, lyophilate or freeze-dried form as provided herein is in a bottle or container, and the liquid is added to the bottle or container, and this mixture can be consumed by an individual in need thereof. In alternative embodiments, a powdered, lyophilate or freeze-dried form as provided herein is in a cartridge that can be part of a container or bottle, and the powdered, lyophilate or freeze-dried form can be mixed with the liquid, for example, as described in U.S. Pat. No. 8,590,753. In alternative embodiments, a powdered, lyophilate or freeze-dried form as provided herein can be contained in or can be added to a container or bottle as described for example, in U.S. Pat. Nos. 10,315,815; 10,315,803; 10,281,317; 10,183,116; 9,809,374; 9,345,831; 9,173,999; 7,874,420.

In alternative embodiments, compositions and formulations as provided herein, and compositions and formulations used to practice methods as provided herein, are formulated for delayed or gradual enteric release using cellulose acetate (CA) and polyethylene glycol (PEG), for example, as described by Defang et al. (2005) Drug Develop. & Indust. Pharm. 31:677-685, who used CA and PEG with sodium carbonate in a wet granulation production process.

In alternative embodiments, compositions and formulations as provided herein, and compositions and formulations used to practice methods as provided herein, are formulated for delayed or gradual enteric release using a hydroxypropylmethylcellulose (HPMC), a microcrystalline cellulose (MCC) and magnesium stearate, as described for example, in Huang et al. (2004) European J. of Pharm. & Biopharm. 58: 607-614).

In alternative embodiments, compositions and formulations as provided herein, and compositions and formulations used to practice methods as provided herein, are formulated for delayed or gradual enteric release using for example, a poly(meth)acrylate, for example a methacrylic acid copolymer B, a methyl methacrylate and/or a methacrylic acid ester, a polyvinylpyrrolidone (PVP) or a PVP-K90 and a EUDRAGIT® RL PO™, as described for example, in Kuksal et al. (2006) AAPS Pharm. 7(1), article 1, E1 to E9.

In alternative embodiments, compositions and formulations as provided herein, and compositions and formulations used to practice methods as provided herein, are formulated for delayed or gradual enteric release as described in U.S. Pat. App. Pub. 20100239667. In alternative embodiments, the composition comprises a solid inner layer sandwiched between two outer layers. The solid inner layer can comprise the non-pathogenic bacteria and/or spores, and one or more disintegrants and/or exploding agents, or one or more effervescent agents or a mixture. Each outer layer can comprise a substantially water soluble and/or crystalline polymer or a mixture of substantially water soluble and/or crystalline polymers, for example, a polyglycol. These can be adjusted to achieve delivery of the living components to the intestine.

In alternative embodiments, compositions and formulations as provided herein, and compositions and formulations used to practice methods as provided herein, are formulated for delayed or gradual enteric release as described in U.S. Pat. App. Pub. 20120183612, which describes stable pharmaceutical formulations comprising active agents in a non-swellable diffusion matrix. In alternative embodiments, compositions and formulations as provided herein, and compositions and formulations used to practice methods as provided herein, are released from a matrix in a sustained, invariant and, if several active agents are present, independent manner and the matrix is determined with respect to its substantial release characteristics by ethylcellulose and at least one fatty alcohol to deliver bacteria distally.

In alternative embodiments, compositions and formulations as provided herein, and compositions and formulations used to practice methods as provided herein, are formulated for delayed or gradual enteric release as described in U.S. Pat. No. 6,284,274, which describes a bilayer tablet containing an active agent (for example, an opiate analgesic), a polyalkylene oxide, a polyvinylpyrrolidone and a lubricant in the first layer and a second osmotic push layer containing polyethylene oxide or carboxy-methylcellulose.

In alternative embodiments, compositions and formulations as provided herein, and compositions and formulations used to practice methods as provided herein, are formulated for delayed or gradual enteric release as described in U.S. Pat. App. Pub. No. 20030092724, which describes sustained release dosage forms in which a nonopioid analgesic and opioid analgesic are combined in a sustained release layer and in an immediate release layer, sustained release formulations comprising microcrystalline cellulose, EUDRAGIT RSPO™, CAB-O-SIL™, sodium lauryl sulfate, povidone and magnesium stearate.

In alternative embodiments, compositions and formulations as provided herein, and compositions and formulations used to practice methods as provided herein, are formulated for delayed or gradual enteric release as described in U.S. Pat. App. Pub. 20080299197, describing a multi-layered tablet for a triple combination release of active agents to an environment of use, for example, in the GI tract. In alternative embodiments, a multi-layered tablet is used, and it can comprise two external drug-containing layers in stacked arrangement with respect to and on opposite sides of an oral dosage form that provides a triple combination release of at least one active agent. In one embodiment the dosage form is an osmotic device, or a gastro-resistant coated core, or a matrix tablet, or a hard capsule. In these alternative embodiments, the external layers may contain biofilm dissolving agents and internal layers can comprise viable/living bacteria, for example, a formulation comprising at least two different species or genera (or types) of non-pathogenic bacteria as used to practice methods as provided herein.

In alternative embodiments, compositions and formulations as provided herein, and compositions and formulations used to practice methods as provided herein, are formulated as multiple layer tablet forms, for example, where a first layer provides an immediate release of a formulation or pharmaceutical preparation as provided herein and a second layer provides a controlled-release of another (or the same) bacteria or drug, or another active agent, for example, as described for example, in U.S. Pat. No. 6,514,531 (disclosing a coated trilayer immediate/prolonged release tablet), U.S. Pat. No. 6,087,386 (disclosing a trilayer tablet), U.S. Pat. No. 5,213,807 (disclosing an oral trilayer tablet with a core comprising an active agent and an intermediate coating comprising a substantially impervious/impermeable material to the passage of the first active agent), and U.S. Pat. No. 6,926,907 (disclosing a trilayer tablet that separates a first active agent contained in a film coat from a core comprising a controlled-release second active agent formulated using excipients which control the drug release, the film coat can be an enteric coating configured to delay the release of the active agent until the dosage form reaches an environment where the pH is above four).

In alternative embodiments, compositions and formulations as provided herein, and compositions and formulations used to practice methods as provided herein, are formulated for delayed or gradual enteric release as described in U.S. Pat. App. Pub. 20120064133, which describes a release-retarding matrix material such as: an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, polyvinylalcohols, polyvinylalcohol copolymers, polyethylene glycols, non-crosslinked polyvinylpyrrolidone, polyvinylacetates, polyvinylacetate copolymers or any combination thereof. In alternative embodiments, spherical pellets are prepared using an extrusion/spheronization technique, of which many are well known in the pharmaceutical art. The pellets can comprise one or more formulations or pharmaceutical preparations as provided herein.

In alternative embodiments, compositions and formulations as provided herein, and compositions and formulations used to practice methods as provided herein, are formulated for delayed or gradual enteric release as described in U.S. Pat. App. Pub. 20110218216, which describes an extended release pharmaceutical composition for oral administration, and uses a hydrophilic polymer, a hydrophobic material and a hydrophobic polymer or a mixture thereof, with a microenvironment pH modifier. The hydrophobic polymer can be ethylcellulose, cellulose acetate, cellulose propionate, cellulose butyrate, methacrylic acid-acrylic acid copolymers or a mixture thereof. The hydrophilic polymer can be polyvinylpyrrolidone, hydroxypropylcellulose, methylcellulose, hydroxypropylmethyl cellulose, polyethylene oxide, acrylic acid copolymers or a mixture thereof. The hydrophobic material can be a hydrogenated vegetable oil, hydrogenated castor oil, carnauba wax, candellia wax, beeswax, paraffin wax, stearic acid, glyceryl behenate, cetyl alcohol, cetostearyl alcohol or and a mixture thereof. The microenvironment pH modifier can be an inorganic acid, an amino acid, an organic acid or a mixture thereof. Alternatively, the microenvironment pH modifier can be lauric acid, myristic acid, acetic acid, benzoic acid, palmitic acid, stearic acid, oxalic acid, malonic acid, succinic acid, adipic acid, sebacic acid, fumaric acid, maleic acid; glycolic acid, lactic acid, malic acid, tartaric acid, citric acid, sodium dihydrogen citrate, gluconic acid, a salicylic acid, tosylic acid, mesylic acid or malic acid or a mixture thereof.

In alternative embodiments, therapeutic combinations or formulations, or pharmaceuticals or the pharmaceutical preparations as provided herein, or as used in methods as provided herein, are formulated as a delayed or gradual enteric release composition or formulation, and optionally the formulation comprises a gastro-resistant coating designed to dissolve at a pH of 7 in the terminal ileum, for example, an active ingredient is coated with an acrylic based resin or equivalent, for example, a poly(meth)acrylate, for example a methacrylic acid copolymer B, NF, which dissolves at pH 7 or greater, for example, comprises a multimatrix (MMX) formulation. In alternative embodiments, compositions and formulations as provided herein, and compositions and formulations used to practice methods as provided herein, are powders that can be included into a suitable carrier, for example, such as a liquid, a tablet or a suppository. In alternative embodiments, compositions and formulations as provided herein, and compositions and formulations used to practice methods as provided herein, are ‘powders for reconstitution’ as a liquid to be drunk, placed down a naso-duodenal tube or used as an enema for patients to take home and self-administer enemas. In alternative embodiments, compositions and formulations as provided herein, and compositions and formulations used to practice methods as provided herein, are micro-encapsulated, formed into tablets and/or placed into capsules, especially enteric-coated capsules. In alternative embodiments, compositions as provided herein are formulated to be effective in a given mammalian subject in a single administration or over multiple administrations. In some embodiments, a substrate or prebiotic required by the bacterial type in a formulation as provided herein is administered for a period of time in advance of the administration of the combination of microbes, for example, bacterial compositions, as provided herein. Such administration (for example, of prebiotics) pre-loads the gastrointestinal tract with the substrates needed by the bacterial types of the composition and increases the potential for the bacterial composition to have adequate resources to perform the required metabolic reactions. In other embodiments, the composition is administered simultaneously with the substrates required by the bacterial types a formulation as provided herein. In still other embodiments the substrate or prebiotic is administered alone. In alternative embodiments, efficacy is measured by an increase in the population of those bacterial types in the subject's intestinal tract, or an increase in the population of those bacterial types originally found in the subject's intestinal tract before treatment.

In alternative embodiments, compositions as provided herein comprise, further comprise, or have added to: at least one probiotic or prebiotic, wherein optionally the prebiotic comprises an inulin, a beta-glucan, a polyol, lactulose, extracts of artichoke, chicory root, oats, barley, various legumes, garlic, kale, beans or flacks or an herb, wherein optionally the probiotic comprises a cultured or stool-extracted microorganism or bacteria, or a bacterial component, and optionally the bacteria or bacterial component comprises or is derived from a Bacteroidetes, a Firmicutes, a Lactobacilli, a Bifidobacteria, an Erysipelatoclostridium, a Ruminococcus, a Clostridium, a Collinsella, an E. coli, a Streptococcus fecalis and equivalents.

In alternative embodiments, compositions as provided herein comprise, further comprise, or have added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch; or, further comprise an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient; or, further comprise, or have added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadorapersica extracts, Competence-stimulating peptide, Patulin and penicillic acid; peptides—cathelicidin-derived peptides, small lytic peptide, PTP-7, nitric oxide, neo-emulsions; ozone, lytic bacteriophages, lactoferrin, xylitol hydrogel, synthetic iron chelators, a statin (optionally lovastatin (optionally MEVACOR™), simvastatin (optionally ZOCOR™), atorvastatin (optionally LIPITOR™), pravastatin (optionally PRAVACHOL™), fluvastain (optionally LESCOL™) or rosuvastatin (optionally CRESTOR™)), cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

In alternative embodiments, compositions as provided herein comprise, further comprise, or have added to: a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.

### Products of Manufacture and Kits

Provided are products of manufacture, for example, implants or pharmaceuticals, and kits, containing components for practicing methods as provided herein, for example, including a formulation comprising a combination of microbes as provided herein, such as for example, freshly isolated microbes, cultured microbes, or genetically engineered microbes, or at least two different species or genera (or types) of non-pathogenic bacteria, wherein each of the non-pathogenic bacteria comprise (or are in the form of) a plurality of non-pathogenic colony forming live bacteria, a plurality of non-pathogenic germinable bacterial spores, or a combination thereof, and optionally including instructions for practicing methods as provided herein.

Companion Diagnostics and Patient Biomarkers

Provided are biomarkers indicative of patient response or non-response to a composition or method as provided herein, including for example, an anti-viral treatment or vaccine, a chemotherapy, a radiation therapy, an immune checkpoint inhibitor (for example, a checkpoint inhibitor therapy), a Chimeric Antigen Receptor (CAR) T-cell therapy (CAR-T) or other immunotherapy or a cancer treatment. These biomarkers may be in the form of microbial species abundance in the gut (or abundance in the colon), microbial gene expression or protein expression, or abundance of a metabolite in a stool sample or a sample of bacteria taken from the gut. Alternatively, the biomarkers may be metabolite concentration, cytokine profile, or protein expression in the blood. These biomarkers are used to develop a diagnostic screen to predict in advance whether a patient will naturally respond to therapy or will require microbial intervention to enable the composition or method as provided herein, for example, checkpoint inhibitors or CAR-T therapy, to function efficaciously or more efficaciously as compared to their effectiveness in the patient if a composition or method as provided herein had not been administered.

Genetic Modification of Microbial Therapeutics

In alternative embodiments, microbes, for example, bacteria, used in compositions as provided herein, or used to practice methods as provided herein, are genetically engineered. In alternative embodiments, microbes are genetically engineered to increase their efficacy, for example, to increase the efficacy of an anti-viral drug or treatment as provided or described herein.

In alternative embodiments, one several or all of a combination of microbes as provided herein, or used to practice methods as provided herein, are genetically engineered. In alternative embodiments, microbes are genetically engineered to substantially decrease, reduce or eliminate their toxicity. In alternative embodiments, microbes are genetically engineered to comprise a kill switch so they can be rendered non-vital after administration of an appropriate trigger or signal. In alternative embodiments, microbes are genetically engineered to secrete anti-inflammatory compositions or have an anti-inflammatory effect. In alternative embodiments, microbes are genetically engineered to secrete an anti-cancer substance.

Microbes, for example, bacteria, used in compositions as provided herein, or used to practice methods as provided herein, can be genetically engineered using any method known in the art, for example, as discussed in the Examples, below. For example, one or more gene sequence(s) and/or gene cassette(s) may be expressed on a high-copy plasmid, a low-copy plasmid, or a chromosome. In some embodiments, expression from the plasmid is used to increase expression of an inserted, for example, heterologous nucleic acid, for example, a gene or protein encoding sequence or an inhibitory nucleic acid such as an antisense or siRNA-encoding nucleic acid. The inserted nucleic acid of interest can be inserted into a bacterial chromosome at one or more integration sites.

For example, in alternative embodiments, microbes are genetically engineered to comprise one or more gene sequence(s) and/or gene cassette(s) for producing a non-native anti-inflammation and/or gut barrier function enhancer molecule. In alternative embodiments, the anti-inflammation and/or gut barrier function enhancer molecule comprises a short-chain fatty acid, butyrate, propionate, acetate, IL-2, IL-22, superoxide dismutase (SOD), GLP-2, GLP-1, IL-10, IL-27, TGF-.beta.1, TGF-.beta.2, N-acylphosphatidylethanolamines (NAPES), elafin (also known as peptidase inhibitor 3 or SKALP), trefoil factor, melatonin, PGD₂, kynurenic acid, and kynurenine. A molecule may be primarily anti-inflammatory, for example, IL-10, or primarily gut barrier function enhancing, for example, GLP-2. In alternative embodiments, microbes are genetically engineered to comprise one or more gene sequence(s) and/or gene cassette(s) that are inhibitory to the activity of, or substantially or completely inhibit expression of, bacterial virulence factors, toxins, or antibiotic resistance functions.

### Bacterial Strains

In alternative embodiments, bacterial strains used in formulations as provided herein, or in methods as provided herein, are identified by their sequence identities to 16S rDNA.

For example, in alternative embodiments, a Clostridium species used in formulations as provided herein, or in methods as provided herein, comprises a 16S rDNA sequence having at least about 90%, 95%, 96%, 97% 98% or 99% or complete sequence identity to SEQ ID NO:1:

Clostridiums sp. AF36-4 16S ribosomal RNA gene
SEQ ID 1
TTTAACATGAGAGTTTGATCCTGGCTCAGGATGAA
CGCTGGCGGCGTGCTTAACACATGCAAGTCGAACG
AAGCACCTCTCCCGAAGATTGACACAGCTTGCTGT
AGATTGATTCATTTGAGGTGACTGAGTGGCGGACG
GGTGAGTAACGCGTGGGTAACCTGCCTCATAGAGG
GGGACAACAGTTGGAAACGACTGCTAATACCGCAT
AGTAAGAAAGATTCGCATGTTTCTTTCTTGAAAGA
TTTATCGCTATGAGATGGACCCGCGTCTGATTAGC
TAGTTGGTAAGGTAACGGCTTACCAAGGCGACGAT
CAGTAGCCGGCTTGAGAGAGTGAACGGCCACATTG
GGACTGAGACACGGCCCAAACTCCTACGGGAGGCA
GCAGTGGGGAATATTGCACAATGGGGGAAACCCTG
ATGCAGCGACGCCGCGTGAGTGAAGAAGTATTTCG
GTATGTAAAGCTCTATCAGCAGGGAAGATAATGAC
GGTACCTGACTAAGAAGCCCCGGCTAACTACGTGC
CAGCAGCCGCGGTAATACGTAGGGGGCAAGCGTTA
TCCGGATTTACTGGGTGTAAAGGGTGCGTAGGTGG
CAAGGCAAGTCAGATGTGAAAGCCCGGGGCTCAAC
CCCGGTACTGCATTTGAAACTGTCTAGCTAGAGTG
CAGGAGAGGTAAGCGGAATTCCTAGTGTAGCGGTG
AAATGCGTAGATATTAGGAGGAACACCAGTGGCGA
AGGCGGCTTACTGGACTGTAACTGACACTGAGGCA
CGAAAGCGTGGGGAGCAAACAGGATTAGATACCCT
GGTAGTCCACGCCGTAAACGATGAATACTAGGTGT
CGGGGCCCACAGGGCTTCGGTGCCGCAGCAAACGC
ATTAAGTATTCCACCTGGGGAGTACGTTCGCAAGA
ATGAAACTCAAAGGAATTGACGGGGACCCGCACAA
GCGGTGGAGCATGTGGTTTAATTCGAAGCAACGCG
AAGAACCTTACCAAGTCTTGACATCCTTCTGACCG
TTCCTTAGCCGGAACTTCCCTTCGGGGCAGAAGTG
ACAGGTGGTGCATGGTTGTCGTCAGCTCGTGTCGT
GAGATGTTGGGTTAAGTCCCGCAACGAGCGCAACC
CTTATCCTTAGTAGCCAGCGGTTCGGCCGGGCACT
CTGGGGAGACTGCCGGAGATAATCCGGAGGAAGGT
GGGGATGACGTCAAATCATCATGCCCCTTATGACT
TGGGCTACACACGTGCTACAATGGCGGTAACAAAG
GGAAGCAGCCTCGCGAGAGTGAGCAAACCCCAAAA
ATGCCGTCTCAGTTCGGATTGTAGTCTGCAACTCG
ACTACATGAAGCTGGAATCGCTAGTAATCGCAGAT
CAGAATGCJGCGGTGAATACGTTCCCGGGTCTTGT
ACACACCGCCCGTCACACCATGGGAGTCGGATATG
CCCGAAGCCAGTGACCCAACCGCAAGGAGGGAGCT
GTCGAAGGTGGAGCCGATAACTGGGGTGAAGTCGT
AACAAGGTAGCCGTATCGGAAGGTGCGGCTGGATC
ACCTCCTTTCTAAGGAA

In alternative embodiments, a Dorea species used in formulations as provided herein, or in methods as provided herein, comprises a 16S rDNA sequence having at least about 90%, 95%, 96%, 97% 98% or 99% or complete sequence identity to SEQ ID NO:2:

Dorea sp. AM58-8 16S ribosomal RNA gene
SEQ ID 2
TTTTTACGAGAGTTTGATCCTGGCTCAGGATGAAC
GCTGGCGGCGTGCTTAACACATGCAAGTCGAACGA
AGCACTTAAGTTTGATTCTTCGGATGAAGACTTTT
GTGACTGAGTGGCGGACGGGTGAGTAACGCGTGGG
TAACCTGCCTCATACAGGGGGATAACAGTTAGAAA
TGACTGCTAATACCGCATAAGACCACAGCACCGCA
TGGTGCAGGGGTAAAAACTCCGGTGGTATGAGATG
GACCCGCGTCTGATTAGCTGGTTGGTGGGGTAACG
GCCTACCAAGCGACGATCAGTAGCCGACCTGAGAG
GGTGACCGGCCACATTGGGACTGAGACACGGCCCA
AACTCCTACGGGAGGCAGCAGTGGGGAATATTGCA
CAATGGGGGAAACCCTGATGCAGCGACGCCGCGTG
AAGGATGAAGTATTTCGGTATGTAAACTTCTATCA
GCAGGGAAGAAAATGACGGTACCTGACTAAGAAGC
CCCGGCTAACTACGTGCCAGCAGCCGCGGTAATAC
GTAGGGGGCAAGCGTTATCCGGATTTACTGGGTGT
AAAGGGAGCGTAGACGGTATGGCAAGTCTGATGTG
AAAGGCCAGGGCTCAACCCTGGGACTGCATTGGAA
ACTGTCGAACTAGAGTGTCGGAGAGGCAAGTGGAA
TTCCTAGTGTAGCGGTGAAATGCGTAGATATTAGG
AGGAACACCAGTGGCGAAGGCGGCTTGCTGGACGA
TGACTGACGTTGAGGCTCGAAAGCGTGGGGAGCAA
ACAGGATTAGATACCCTGGTAGTCCACGCCGTAAA
CGATGACTACTAGGTGTCGGGTAGCAGAGCTATTC
GGTGCCGCAGCCAACGCAATAAGTAGTCCACCTGG
GGAGTACGTTCGCAAGAATGAAACTCAAAGGAATT
GACGGGGACCCGCACAAGCGGTGGAGCATGTGGTT
TAATTCGAAGCAACGCGAAGAACCTTACCTGCTCT
TGACATCTCCCTGACCGGCAAGTAATGTTGCCTTT
CCTTCGGGACAGGGATGACAGGTGGTGCATGGTTG
TCGTCAGCTCGTGTCGTGAGATGTTGGGTTAAGTC
CCGCAACGAGCGCAACCCCTATCTTTAGTAGCCAG
CGGTTCGGCCGGGCACTCTAGAGAGACTGCCAGGG
ATAACCTGGAGGAAGGTGGGGATGACGTCAAATCA
TCATGCCCCTTATGAGCAGGGCTACACACGTGCTA
CAATGGCGTAAACAAAGGGAAGCGAGCCTGCGAGG
GTAAGCAAATCTCAAAAATAACGTCTCAGTTCGGA
TTGTAGTCTGCAACTCGACTACATGAAGCTGGAAT
CGCTAGTAATCGCGAATCAGAATGTCGCGGTGAAT
ACGTTCCCGGGTCTTGTACACACCGCCCGTCACAC
CATGGGAGTTGGTAACGCCCGAAGTCAGTGACCCA
ACCGCAAGGAGGGAGCTGCCGAAGGTGGGACCGAT
AACTGGGGTGAAGTCGTAACAAGGTAGCCGTATCG
GAAGGTGCGGCTGGATCACCTCCTTTCTAAGGAA

In alternative embodiments, an Erysipelotrichaceae species used in formulations as provided herein, or in methods as provided herein, comprises a 16S rDNA sequence having at least about 90%, 95%, 96%, 97% 98% or 99% or complete sequence identity to SEQ ID NO:3:

Erysipelotrichaceae bacterium GAM147
16S ribosomal RNA gene
SEQ ID 3
AATGGAGAGTTTGATCCTGGCTCAGGATGAACGCT
GGCGGCGTGCCTAATACATGCAAGTCGAACGCTTC
ACTTCGGTGAAGAGTGGCGAACGGGTGAGTAATAC
ATAAGTAACCTGGCATCTACAGGGGGATAACTGAT
GGAAACGTCAGCTAAGACCGCATAGGTGTAGAGAT
CGCATGAACTCTATATGAAAAGTGCTACGGGACTG
GTAGATGATGGACTTATGGCGCATTAGCTTGTTGG
TAGGGTAACGGCCTACCAAGGCGACGATGCGTAGC
CGACCTGAGAGGGTGACCGGCCACACTGGGACTGA
GACACGGCCCAGACTCCTACGGGAGGCAGCAGTAG
GGAATTTTCGGCAATGGGGGAAACCCTGACCGAGC
AACGCCGCGTGAAGGAAGAAGTAATTCGTTATGTA
AACTTCTGTCATAGAGGAAGAACGGTGGATATAGG
AAATGATATCCAAGTGACGGTACTCTATAAGAAAG
CCACGGCTAACTACGTGCCAGCAGCCGCGGTAATA
CGTAGGTGGCGAGCGTTATCCGGAATTATTGGGCG
TAAAGAGGGAGCAGGCGGCACTAAGGGTCTGTGGT
GAAAGATCGAAGCTTAACTTCGGTAAGCCATGGAA
ACCGTAGAGCTAGAGTGTGTGAGAGGATCGTGGAA
TTCCATGTGTAGCGGTGAAATGCGTAGATATATGG
AGGAACACCAGTGGCGAAGGCGACGATCTGGCGCA
TAACTGACGCTCAGTCCCGAAAGCGTGGGGAGCAA
ATAGG
ATTAGATACCCTAGTAGTCCACGCCGTAAACGATG
AGTACTAAGTGTTGGGGGTCAAACCTCAGTGCTGC
AGTTAACGCAATAAGTACTCCGCCTGAGTAGTACG
TTCGCAAGAATGAAACTCAAAGGAATTGACGGGGG
CCCGCACAAGCGGTGGAGCATGTGGTTTAATTCGA
AGCAACGCGAAGAACCTTACCAGGTCTTGACATCG
ATCTAAAGGCTCCAGAGATGGAGAGATAGCTATAG
AGAAGACAGGTGGTGCATGGTTGTCGTCAGCTCGT
GTCGTGAGATGTTGGGTTAAGTCCCGCAACGAGCG
CAACCCCTGTTGCCAGTTGCCAGCATTAAGTTGGG
GACTCTGGCGAGACTGCCGGTGACAAGCCGGAGGA
AGGCGGGGATGACGTCAAATCATCATGCCCCTTAT
GACCTGGGCTACACACGTGCTACAATGGACAGAGC
AGAGGGAAGCGAAGCCGCGAGGTGGAGCGAAACCC
ATAAAACTGTTCTCAGTTCGGACTGCAGTCTGCAA
CTCGACTGCACGAAGATGGAATCGCTAGTAATCGCG
ATCAGCATGTCGCGGTGAATACGTTCTCGGGCCTT
AGTACACACCGCCCGTCACACCATGAGAGTCGGTAA
CACCCGAAGCCGGTGGCCTAACCGCAAGGAAGGAG
CTGTCTAAGGTGGGACTGATGATTGGGGTGAAGTC
GTAACAAGGTATCCCTACGGGAACGTGGGGATGGA
TCACCTCCTTTCTAGGGAGA

In alternative embodiments, a Firmicutes species used in formulations as provided herein, or in methods as provided herein, comprises a 16S rDNA sequence having at least about 90%, 95%, 96%, 97% 98% or 99% or complete sequence 50 identity to SEQ ID NO:4:

Firmicutes bacterium AF12-30 16S
ribosomal RNA gene
SEQ ID 4
GAACATGAGAGTTTGATCCTGGCTCAGGATGAACGC
TGGCGGCGTGCTTAACACATGCAAGTCGAACGAAG
CGCCTTATTTGATTTTCTTCGGAACTGAAGATTTG
GTGACTGAGTGGCGGACGGGTGAGTAACGCGTGGG
TAACCTGCCCTGTACAGGGGGATAACAATCAGAAA
TGACTGCTAATACCGCATAAGACCACAGCACCGCA
TGGTGCAGGGGTAAAAACTCCGGTGGTACAGGATG
GACCCGCGTCTGATTAGCTGGTTGGTGAGGTAACG
GCTCACCAAGGCGACGATCAGTAGCCGGCTTGAGA
GAGTGAACGGCCACATTGGGACTGAGACACGGCCC
AAACTCCTACGGGAGGCAGCAGTGGGGAATATTGC
ACAATGGGGGGAACCCTGATGCAGCGACGCCGCGT
GAGTGAAGAAGTATCTCGGTATGTAAAGCTCTATC
AGCAGGGAAGAAAATGACGGTACCTGACTAAGAAG
CCCCGGCTAACTACGTGCCAGCAGCCGCGGTAATA
CGTAGGGGGCAAGCGTTATCCGGAATTACTGGGTG
TAAAGGGTGCGTAGGTGGTATGGCAAGTCAGAAGT
GAAAACCCAGGGCTTAACTCTGGGACTGCTTTTGA
AACTGTCAGACTGGAGTGCAGGAGAGGTAAGCGGA
ATTCCTAGTGTAGCGGTGAAATGCGTAGATATTAG
GAGGAACATCAGTGGCGAAGGCGGCTTACTGGACT
GAAACTGACACTGAGGCACGAAAGCGTGGGGAGCA
AACAGGATTAGATACCCTGGTAGTCCACGCCGTAA
ACGATGAATACTAGGTGTCGGGGCCGTAGAGGCTT
CGGTGCCGCAGCCAACGCAGTAAGTATTCCACCTG
GGGAGTACGTTCGCAAGAATGAAACTCAAAGGAAT
TGACGGGGACCCGCACAAGCGGTGGAGCATGTGGT
TTAATTCGAAGCAACGCGAAGAACCTTACCTGGTC
TTGACATCCTTCTGACCGGTCCTTAACCGGACCTT
TCCTTCGGGACAGGAGTGACAGGTGGTGCATGGTT
GTCGTCAGCTCGTGTCGTGAGATGTTGGGTTAAGT
CCCGCAACGAGCGCAACCCCTATCTTTAGTAGCCA
GCATTTCAGGTGGGCACTCTAGAGAGACTGCCAGG
GATAACCTGGAGGAAGGTGGGGACGACGTCAAATC
ATCATGCCCCTTATGACCAGGGCTACACACGTGCT
ACAATGGCGTAAACAGAGGGAAGCAGCCTCGTGAG
AGTGAGCAAATCCCAAAAATAACGTCTCAGTTCGG
ATTGTAGTCTGCAACTCGACTACATGAAGCTGGAA
TCGCTAGTAATCGCGAATCAGAATGTCGCGGTGAA
TACGTTCCCGGGTCTTGTACACACCGCCCGTCACA
CCATGGGAGTCAGTAACGCCCGAAGTCAGTGACCC
AACCGTAAGGAGGGAGCTGCCGAAGGCGGGACCGA
TAACTGGGGTGAAGTCGTAACAAGGTAGCCGTATC
GGAAGGTGCGGCTGGATCACCTCCTTTCTAAGGAA

In alternative embodiments, a Ruminococcus species used in formulations as provided herein, or in methods as provided herein, comprises a 16S rDNA sequence having at least about 90%, 95%, 96%, 97% 98% or 99% or complete sequence identity to SEQ ID NO:5:

Ruminococcus sp. OF03-6AA 16S
ribosomal RNA gene
SEQ ID 5
TTCTAGTGGCGGACGGGTGAGTAACGCGTGGGTAA
CCTGCCTTGTACAGGGGGATAACAGTCAGAAATGA
CTGCTAATACCGCATAAGCGCACAGGACCGCATGG
TCCGGTGTGAAAAACTCCGGTGGTATAAGATGGAC
CCGCGTTGGATTAGCTAGTTGGCAGGGTAACGGCC
TACCAAGCGACGATCCATAGCCGGCCTGAGAGGGT
GAACGGCCACATTGGGACTGAGACACGGCCCAGAC
TCCTACGGGAGGCAGCAGTGGGGAATATTGCACAA
TGGGGGAAACCCTGATGCAGCGACGCCGCGTGAAG
GAAGAAGTATCTCGGTATGTAAACTTCTATCAGC
AGGGAAGAAAATGACGGTACCTGACT
AAGAAGCCCCGGCTAACTACGTGCCAGCAGCCGCG
GTAATACGTAGGGGGCAAGCGTTATCCGGATTTAC
TGGGTGTAAAGGGAGCGTAGACGGATGGACAAGTC
TGATGTGAAAGGCTGGGGCTCAACCCCGGGACTGC
ATTGGAAACTGCCCGTCTTGAGTGCCGGAGAGGTA
AGCGGAATTCCTAGTGTAGCGGTGAAATGCGTAGA
TATTAGGAGGAACACCAGTGGCGAAGGCGGCTTAC
TGGACGGTAACTGACGTTGAGGCTCGAAAGCGTGG
GGAGCAAACAGGATTAGATACCCTGGTAGTCCACG
CGGTAAACGATGAATGCTAGGTGTCGGGTGACAAA
GTCATTCGGTGCCGCCGCAAACGCATTAAGCATTC
CACCTGGGGAGTACGTTCGCAAGAATGAAACTCAA
AGGAATTGACGGGGACCCGCACAAGCGGTGGAGCA
TGTGGTTTAATTCGAAGCAACGCGAAGAACCTTAC
CAAGTCTTGACATCCCTCTGACCGGAACTTAACCG
TTCCTTCCCTTCGGGGCAGAGGAGACAGGTGGTGC
ATGGTTGTCGTCAGCTCGTGTCGTGAGATGTTGGG
TTAAGTCCCGCAACGAGCGCAACCCCTATCCTCAG
TAGCCAGCAGTTCGGCTGGGCACTCTGTGGAGACT
GCCAGGGATAACCTGGAGGAAGGC
GGGGATGACGTCAAATCATCATGCCCCTTATGATT
TGGGCTACACACGTGCTACAATGGCGTAAACAAAG
GGAAGCGAACCTGCGAGGGTGGGCAAATCCCAAAA
ATAACGTCCCAGTTCGGACTGTAGTCTGCAACCCG
ACTACACGAAGCTGGAATCGCTAGTAATCGCGGAT
CAG
AATGCCGCGGTGAATACGTTCCCGGGTCTTGTACA
CACCGCCCGTCACACCATGGGAGTCAGTAACGCCC
GAAGTCAGTGACCTAACCGTAAGGAGGGAGCTGCC
GAAGGCGGGACCGATGACTGGGGTGAAGTCGTAAC
AAGGTAGCCGTATCGGAAGGTGCGGCTGGATCACC
TCCTTTCTAAGGAA

In alternative embodiments, a Collinsella species used in formulations as provided herein, or in methods as provided herein, comprises a 16S rDNA sequence having at least about 90%, 95%, 96%, 97% 98% or 99% or complete sequence identity to SEQ ID NO:6:

Collinsella sp. AM34-10 16S
ribosomal RNA gene
SEQ ID 6
TTTGGACGGAGAGTTCGATCCTGGCTCAGGATGAA
CGCTGGCGGCGCGCCTAACACATGCAAGTCGAACG
GCACCTGCCTTCGGGCAGAAGCGAGTGGCGAACGG
CTGAGTAACACGTGGAGAACCTGCCCCCTCCCCCG
GGATAGCCGCCCGAAAGGACGGGTAATACCGGATA
CCCCGGGGTGCCGCATGGCACCCCGGCTAAAGCCC
CGACGGGAGGGGATGGCTCCGCGGCCCATCAGGTA
GACGGCGGGGTGACGGCCCACCGTGCCGACAACGG
GTAGCCGGGTTGAGAGACCGACCGGCCAGATTGGG
ACTGAGACACGGCCCAGACTCCTACGGGAGGCAGC
AGTGGGGAATCTTGCGCAATGGGGGGAACCCTGAC
GCAGCGACGCCGCGTGCGGGACGGAGGCCTTCGGG
TCGTAAACCGCTTTCAGCAGGGAAGAGTCAAGACT
GTACCTGCAGAAGAAGCCCCGGCTAACTACGTGCC
AGCAGCCGCGGTAATACGTAGGGGGCGAGCGTTAT
CCGGATTCATTGGGCGTAAAGCGCGCGTAGGCGGC
CCGGCAGGCCGGGGGTCGAAGCGGGGGGCTCAACC
CCCCGAAGCCCCCGGAACCTCCGCGGCTTGGGTCC
GGTAGGGGAGGGTGGAACACCCGGTGTAGCGGTGG
AATGCGCAGATATCGGGTGGAACACCGGTGGCGAA
GGCGGCCCTCTGGGCCGAGACCGACGCTGAGGCGC
GAAAGCTGGGGGAGCGAACAGGATTAGATACCCTG
GTAGTCCCAGCCGTAAACGATGGACGCTAGGTGTG
GGGGGACGATCCCCCCGTGCCGCAGCCAACGCATT
AAGCGTCCCGCCTGGGGAGTACGGCCGCAAGGCTA
AAACTCAAAGGAATTGACGGGGGCCCGCACAAGCA
GCGGAGCATGTGGCTTAATTCGAAGCAACGCGAAG
AACCTTACCAGGGCTTGACATATGGGTGAAGCGGG
GGAGACCCCGTGGCCGAGAGGAGCCCATACAGGTG
GTGCATGGCTGTCGTCAGCTCGTGTCGTGAGATGT
TGGGTTAAGTCCCGCAACGAGCGCAACCCCCGCCG
CGTGTTGCCATCGGGTGATGCCGGGAACCCACGCG
GGACCGCCGCCGTCAAGGCGGAGGAGGGCGGGGAC
GACGTCAAGTCATCATGCCCCTTATGCCCTGGGCT
GCACACGTGCTACAATGGCCGGTACAGAGGGATGC
CACCCCGCGAGGGGGAGCGGATCCCGGAAAGCCGG
CCCCAGTTCGGATTGGGGGCTGCAACCCGCCCCCA
TGAAGTCGGAGTTGCTAGTAATCGCGGATCAGCAT
GCCGCGGTGAATGCGTTCCCGGGCCTTGTACACAC
CGCCCGTCACACCACCCGAGTCGTCTGCACCCGAA
GTCGCCGGCCCAACCGCAAGGGGGGAGGCGCCGAA
GGTGTGGAGGGTGAGGGGGGTGAAGTCGTAACAAG
GTAGCCGTACCGGAAGGTGCGGCTGGATCACCTCC
TTTCTAGGGAG

In alternative embodiments, a Coprobacillus species used in formulations as provided herein, or in methods as provided herein, comprises a 16S rDNA sequence having at least about 90%, 95%, 96%, 97% 98% or 99% or complete sequence identity to SEQ ID NO:7:

Coprobacillus sp. 8_1_38FAA 16S ribosomal
RNA gene
SEQ ID 7
AATGGAGAGTTTGATCCTGGCTCAGGATGAACGCTGGCGGCGTGCCTAAT
ACATGCAAGTCGAACGCTTCACTTCGGTGAAGAGTGGCGAACGGGTGAGT
AATACATAAGTAACCTGGCCTTTACAGGGGGATAACTATTGGAAACGATA
GCTAAGACCGCATAGGTGTCAAAACCGCATGGAGATGACATGAAATATGC
TACGGCATAGGTAGAGGATGGACTTATGGCGCATTAGCTAGTTGGAGGGG
TAACGGCCCACCAAGGCGACGATGCGTAGCCGACCTGAGAGGGTGACCGG
CCACACTGGGACTGAGACACGGCCCAGACTCCTACGGGAGGCAGCAGTAG
GGAATTTTCGGCAATGGGGGAAACCCTGACCGAGCAACGCCGCGTGAAGG
AAGAAGTAATTCGTTATGTAAACTTCTGTCATAGAGGAAGAACGGTGCGT
GTAGGGAATGACATGCAAGTGACGGTACTCTATAAGAAAGCCACGGCTAA
CTACGTGCCAGCAGCCGCGGTAATACGTAGGTGGCGAGCGTTATCCGGAA
TTATTGGGCGTAAAGAGGGAGCAGGCGGCACTAAGGGTCTGTGGTGAAAG
ATCGAAGCTTAACTTCGGTAAGCCATGGAAACCGTAGAGCTAGAGTGTGT
GAGAGGATCGTGGAATTCCATGTGTAGCGGTGAAATGCGTAGATATATGG
AGGAACACCAGTGGCGAAGGCGACGATCTGGCGCATAACTGACGCTCAGT
CCCGAAAGCGTGGGGAGCAAATAGGATTAGATACCCTAGTAGTCCACGCC
GTAAACGATGAGTACTAAGTGTTGGGAGTCAAATCTCAGTGCTGCAGTTA
ACGCAATAAGTACTCCGCCTGAGTAGTACGTTCGCAAGAATGAAACTCAA
AGGAATTGACGGGGGCCCGCACAAGCGGTGGAGCATGTGGTTTAATTCGA
AGCAACGCGAAGAACCTTACCAGGTCTTGACATCGATCTAAAGGCTCCAG
AGATGGAGAGATAGCTATAGAGAAGACAGGTGGTGCATGGTTGTCGTCAG
CTCGTGTCGTGAGATGTTGGGTTAAGTCCCGCAACGAGCGCAACCCCTGT
TGCCAGTTGCCAGCATTAAGTTGGGGACTCTGGCGAGACTGCCGGTGACA
AGCCGGAGGAAGGCGGGGATGACGTCAAATCATCATGCCCCTTATGACCT
GGGCTACACACGTGCTACAATGGACAGAGCAGAGGGAAGCGAAGCCGCGA
GGTGGAGCGAAACCCAGAAAACTGTTCTCAGTTCGGACTGCAGTCTGCAA
CTCGACTGCACGAAGTTGGAATCGCTAGTAATCGCGAATCAGCATGTCGC
GGTGAATACGTTCTCGGGCCTTGTACACACCGCCCGTCACACCATGAGAG
TCGGTAACACCCGAAGCCGGTGGCCTAACCGCAAGGAAGGAGCTGTCTAA
GGTGGGACTGATGATTGGGGTGAAGTCGTAACAAGGTATCCCTACGGGAA
CGTGGGGATGGATCACCTCCTTTCTAGGGAGA

In alternative embodiments, a Dorea species used in formulations as provided herein, or in methods as provided herein, comprises a 16S rDNA sequence having at least about 90%, 95%, 96%, 97% 98% or 99% or complete sequence identity to SEQ ID NO:8:

Dorea sp. OM07-5 16S ribosomal RNA gene
SEQ ID 8
TTTAACGAGAGTTTGATCCTGGCTCAGGATGAACGCTGGCGGCGTGCTTA
ACACATGCAAGTCGAGCGAAGCACCTAAGAAAGATTCTTCGGATGAATTC
TTTTGTGACTGAGCGGCGGACGGGTGAGTAACGCGTGGGTAACCTGCCTC
ATACAGGGGGATAACAGTTAGAAATGACTGCTAATACCGCATAAGACCAC
GGTACTGCATGGTACAGTGGTAAAAACTCCGGTGGTATGAGATGGACCCG
CGTCTGATTAGCTAGTTGGTGGGGTAACGGCCTACCAAGGCGACGATCAG
TAGCCGACCTGAGAGGGTGACCGGCCACATTGGGACTGAGACACGGCCCA
AACTCCTACGGGAGGCAGCAGTGGGGAATATTGCACAATGGGGGAAACCC
TGATGCAGCGACGCCGCGTGAAGGATGAAGTATTTCGGTATGTAAACTTC
TATCAGCAGGGAAGAAAATGACGGTACCTGACTAAGAAGCCCCGGCTAAC
TACGTGCCAGCAGCCGCGGTAATACGTAGGGGGCAAGCGTTATCCGGATT
TACTGGGTGTAAAGGGAGCGTAGACGGCATTGCAAGCCAGATGTGAAAGC
CCGGGGCTCAACCCCGGGACrGCATTTGGACCGGCAGGCCGGGGGTCGAA
GCGGGGGGCTCAACCCCCCGAAGCCCCCGGAACCTCCGCGGCTTGGGTCC
GGTAGGGGAGGGTGGAACACCCGGTGTAGCGGTGGAATGCGCAGATATCG
GGTGGAACACCGGTGGCGAAGGCGGCCCTCTGGGCCGAGACCGACGCTGA
GGCGCGAAAGCTGGGGGAGCGAACAGGATTAGATACCCrGGTAGTCCCAG
CCGTAAACGATGGACGCTAGGTGTGGGGGGACGATCCCCCCGTGCCGCAG
CCAACGCATTAAGCGTCCCGCCTGGGGAGTACGGCCGCAAGGCTAAAACT
CAAAGGAATTGACGGGGGCCCGCACAAGCAGCGGAGCATGTGGCTTAATT
CGAAGCAACGCGAAGAACCTTACCAGGGCTTGACATATGGGTGAAGCGGG
GGAGACCCCGTGGCCGAGAGGAGCCCATACAGGTGGTGCATGGCTGTCGT
CAGCTCGTGTCGTGAGATGTTGGGTTAAGTCCCGCAACGAGCGCAACCCC
CGCCGCGTGTTGCCATCGGGTGATGCCGGGAACCCACGCGGGACCGCCGC
CGTCAAGGCGGAGGAGGGCGGGGACGACGTCAAGTCATCATGCCCCTTAT
GCCCTGGGCTGCACACGTGCTACAATGGCCGGTACAGAGGGATGCCACCC
CGCGAGGGGGAGCGGATCCCGGAAAGCCGGCCCCAGTTCGGATTGGGGGC
TGCAACCCGCCCCCATGAAGTCGGAGTTGCTAGTAATCGCGGATCAGCAT
GCCGCGGTGAATGCGTTCCCGGGCCTTGTACACACCGCCCGTCACACCAC
CCGAGTCGTCTGCACCCGAAGTCGCCGGCCCAACCGCAAGGGGGGAGGCG
CCGAAGGTGTGGAGGGTGAGGGGGGTGAAGTCGTAACAAGGTAGCCGTAC
CGGAAGGTGCGGCTGGATCACCTCCTTTCTAGGGAG

In alternative embodiments, a Faecalibacterium prausnitzii species used in formulations as provided herein, or in methods as provided herein, comprises a 16S rDNA sequence having at least about 90%, 95%, 96%, 97% 98% or 99% or complete sequence identity to SEQ ID NO:9:

Faecalibacterium prausnitzii A2-165
16S ribosomal RNA gene
SEQ ID 9
TCCTTAGAAAGGAGGTGATCCAGCCGCAGGTTCTCCTACGGCTACCTTGT
TACGACTTCACCCCAATCACCAGTTTTACCTTCGGCGGCGTCCTCCTTGC
GGTTAGACTACCGACTTCGGGTCCCCCCGGCTCTCATGGTGTGACGGGCG
GTGTGTACAAGGCCCGGGAACGTATTCACCGTGGCATGCTGATCCACGAT
TACTAGCAATTCCGACTTCGTGCAGGCGAGTTGCAGCCTGCAGTCCGAAC
TGGGACGTTGTTTCTGAGTTTTGCTCCACCTCGCGGTCTTGCTTCTCTTT
GTTTAACGCCATTGTAGTACGTGTGTAGCCCAAGTCATAAAGGGCATGAT
GATTTGACGTCATCCCCACCTTCCTCCGTTTTGTCAACGGCAGTCCTGCC
AGAGTCCTCTTGCGTAGTAACTGACAGTAAGGGTTGCGCTCGTTGCGGGA
CTTAACCCAACATCTCACGACACGAGCTGACGACAACCATGCACCACCTG
TCTCTGCGTCCCGAAGGAAAATACTGTTTCCAGCATCGTCGCAGGATGTC
AAGACTTGGTAAGGTTCTTCGCGTTGCGTCGAATTAAACCACATACTCCA
CTGCTTGTGCGGGCCCCCGTCAATTCCTTTGAGTTTCAACCTTGCGGTCG
TACTCCCCAGGTGGATTACTTATTGTGTTAACTGCGGCACTGAAGGGGTC
AATCCTCCAACACCTAGTAATCATCGTTTACGGTGTGGACTACCAGGGTA
TCTAATCCTGTTTGCTACCCACACTTTCGAGCCTCAGCGTCAGTTGGTGC
CCAGTAGGCCGCCTTCGCCACTGGTGTTCCTCCCGATATCTACGCATTCC
ACCGCTACACCGGGAATTCCGCCTACCTCTGCACTACTCAAGAAAAACAG
TTTTGAAAGCAGTTTATGGGTTGAGCCCATAGATTTCACTTCCAACTTGT
CTTCCCGCCTGCGCTCCCTTTACACCCAGTAATTCCGGACAACGCTTGTG
ACCTACGTTTTACCGCGGCTGCTGGCACGTAGTTAGCCGTCACTTCCTTG
TTGAGTACCGTCATTATCTTCCTCAACAACAGGAGTTTACAATCCGAAGA
CCTTCTTCCTCCACGCGGCGTCGCTGCATCAGGGTTTCCCCCATTGTGCA
ATATTCCCCACTGCTGCCTCCCGTAGGAGTCTGGGCCGTGTCTCAGTCCC
AATGTGGCCGTTCAACCTCTCAGTCCGGCTACCGATCGTCGCCTTGGTGG
GCCATTACCTCACCAACTAGCTAATCGGACGCGAGGCCATCTCAAAGCGG
ATTGCTCCTTTTCCCTCTGCTCGATGCCGAGCTGTGGGCTTATGCGGTAT
TAGCAGTCGTTTCCAACTGTTGTCCCCCTCTTTGAGGCAGGTTCCTCACG
CGTTACTCACCCGTTCGCCACTCGCTTGAGAAAGCAAGCTCTCTCTCGCT
CGTTCGACTTGCATGTGTTAGGCGCGCCGCCAGCGTTCGTCCTGAGCCAG
GATCAAACTCTTTATAAA

In alternative embodiments, a Clostridium coccoides species used in formulations as provided herein, or in methods as provided herein, comprises a 16S rDNA sequence having at least about 90%, 95%, 96%, 97% 98% or 99% or complete sequence identity to SEQ ID NO:10:

Clostridium coccoides strain 8F 16S
ribosomal RNA gene
SEQ ID 10
TTTGAGTTTGATCCTGGCTCAGGATGAACGCTGGCGGCGTGCTTAACACA
TGCAAGTCGAGCGAAGCGCTAAGACAGATTTCTTCGGATTGAAGTCTTTG
TGACTGAGCGGCGGACGGGTGAGTAACGCGTGGGTAACCTGCCTCATACA
GGGGGATAACAGTTAGAAATGACTGCTAATACCGCATAAGCGCACAGGAC
CGCATGGTCTGGTGTGAAAAACTCCGGTGGTATGAGATGGACCCGCGTCT
GATTAGCTAGTTGGAGGGGTAACGGCCCACCAAGGCGACGATCAGTAGCC
GGCCTGAGAGGGTGAACGGCCACATTGGGACTGAGACACGGCCCAGACTC
CTACGGGAGGCAGCAGTGGGGAATATTGCACAATGGGGGAAACCCTGATG
CAGCGACGCCGCGTGAAGGAAGAAGTATCTCGGTATGTAAACTTCTATCA
GCAGGGAAGAAAATGACGGTACCTGACTAAGAAGCCCCGGCTAACTACGT
GCCAGCAGCCGCGGTAATACGTAGGGGGCAAGCGTTATCCGGATTTACTG
GGTGTAAAGGGAGCGTAGACGGAAGAGCAAGTCTGATGTGAAAGGCTGGG
GCTTAACCCCAGGACTGCATTGGAAACTGTTGTTCTAGAGTGCCGGAGAG
GTAAGCGGAATTCCTAGTGTAGCGGTGAAATGCGTAGATATTAGGAGGAA
CACCAGTGGCGAAGGCGGCTTACTGGACGGTAACTGACGTTGAGGCTCGA
AAGCGTGGGGAGCAAACAGGATTAGATACCCTGGTAGTCCACGCCGTAAA
CGATGAATACTAGGTGTCGGGTGGCAAAGCCATTCGGTGCCGCAGCAAAC
GCAATAAGTATTCCACCTGGGGAGTACGTTCGCAAGAATGAAACTCAAAG
GAATTGACGGGGACCCGCACAAGCGGTGGAGCATGTGGTTTAATTCGAAG
CAACGCGAAGAACCTTACCAAGTCTTGACATCCCTCTGACCGTCCCGTAA
CGGGGGCTTCCCTTCGGGGCAGAGGAGACAGGTGGTGCATGGTTGTCGTC
AGCTCGTGTCGTGAGATGTTGGGTTAAGTCCCGCAACGAGCGCAACCCTT
ATCCTTAGTAGCCAGCACATGATGGTGGGCACTCTAGGGAGACTGCCGGG
GATAACCCGGAGGAAGGCGGGGACGACGTCAAATCATCATGCCCCTTATG
ATTTGGGCTACACACGTGCTACAATGGCGTAAACAAAGGGAAGCGAGACA
GCGATGTTGAGCGAATCCCAAAAATAACGTCCCAGTTCGGACTGCAGTCT
GCAACTCGACTGCACGAAGCTGGAATCGCTAGTAATCGCGGATCAGAATG
CCGCGGTGAATACGTTCCCGGGTCTTGTACACACCGCCCGTCACACCATG
GGAGTCAGTAACGCCCGAAGTCAGTGACCTAACCGAAAGGAAGGAGCTGC
CGAAGGCGGGACCGATAACTGGGGTGAAGTCGTAACAAGGTAGCCGTATC
GGAAGGTGCGGCTGGATCCCC

In alternative embodiments, a Bifidobacterium bifidum species used in formulations as provided herein, or in methods as provided herein, comprises a 16S rDNA sequence having at least about 90%, 95%, 96%, 97% 98% or 99% or complete sequence identity to SEQ ID NO:11:

Bifidobacterium bifidum NCIMB 41171 16S
ribosomal RNA gene
SEQ ID 11
TTTTGTGGAGGGTTCGATTCTGGCTCAGGATGAACGCTGGCGGCGTGCTT
AACACATGCAAGTCGAACGGGATCCATCAAGCTTGCTTGGTGGTGAGAGT
GGCGAACGGGTGAGTAATGCGTGACCGACCTGCCCCATGCTCCGGAATAG
CTCCTGGAAACGGGTGGTAATGCCGGATGTTCCACATGATCGCATGTGAT
TGTGGGAAAGATTCTATCGGCGTGGGATGGGGTCGCGTCCTATCAGCTTG
TTGGTGAGGTAACGGCTCACCAAGGCTTCGACGGGTAGCCGGCCTGAGAG
GGCGACCGGCCACATTGGGACTGAGATACGGCCCAGACTCCTACGGGAGG
CAGCAGTGGGGAATATTGCACAATGGGCGCAAGCCTGATGCAGCGACGCC
GCGTGAGGGATGGAGGCCTTCGGGTTGTAAACCTCTTTTGTTTGGGAGCA
AGCCTTCGGGTGAGTGTACCTTTCGAATAAGCGCCGGCTAACTACGTGCC
AGCAGCCGCGGTAATACGTAGGGCGCAAGCGTTATCCGGATTTATTGGGC
GTAAAGGGCTCGTAGGCGGCTCGTCGCGTCCGGTGTGAAAGTCCATCGCT
TAACGGTGGATCTGCGCCGGGTACGGGCGGGCTGGAGTGCGGTAGGGGAG
ACTGGAATTCCCGGTGTAACGGTGGAATGTGTAGATATCGGGAAGAACAC
CGATGGCGAAGGCAGGTCTCTGGGCCGTCACTGACGCTGAGGAGCGAAAG
CGTGGGGAGCGAACAGGATTAGATACCCTGGTAGTCCACGCCGTAAACGG
TGGACGCTGGATGTGGGGCACGTTCCACGTGTTCCGTGTCGGAGCTAACG
CGTTAAGCGTCCCGCCTGGGGAGTACGGCCGCAAGGCTAAAACTCAAAGA
AATTGACGGGGGCCCGCACAAGCGGCGGAGCATGCGGATTAATTCGATGC
AACGCGAAGAACCTTACCTGGGCTTGACATGTTCCCGACGACGCCAGAGA
TGGCGTTTCCCTTCGGGGCGGGTTCACAGGTGGTGCATGGTCGTCGTCAG
CTCGTGTCGTGAGATGTTGGGTTAAGTCCCGCAACGAGCGCAACCCTCGC
CCCGTGTTGCCAGCACGTTATGGTGGGAACTCACGGGGGACCGCCGGGGT
TAACTCGGAGGAAGGTGGGGATGACGTCAGATCATCATGCCCCTTACGTC
CAGGGCTTCACGCATGCTACAATGGCCGGTACAGCGGGATGCGACATGGC
GACATGGAGCGGATCCCTGAAAACCGGTCTCAGTTCGGATCGGAGCCTGC
AACCCGGCTCCGTGAAGGCGGAGTCGCTAGTAATCGCGGATCAGCAACGC
CGCGGTGAATGCGTTCCCGGGCCTTGTACACACCGCCCGTCAAGTCATGA
AAGTGGGCAGCACCCGAAGCCGGTGGCCTAACCCCTTGTGGGATGGAGCC
GTCTAAGGTGAGGCTCGTGATTGGGACTAAGTCGTAACAAGGTAGCCGTA
CCGGAAGGTGCGGCTGGATCACCTCCTTTCTACGGAG

In alternative embodiments, a Ruminococcus lactaris species used in formulations as provided herein, or in methods as provided herein, comprises a 16S rDNA sequence having at least about 90%, 95%, 96%, 97% 98% or 99% or complete sequence identity to SEQ ID NO:12:

Ruminococcus lactaris 16S ribosomal RNA gene
SEQ ID 12
TTTAACGAGAGTTTGATCCTGGCTCAGGATGAACGCTGGCGGCGTGCTTA
ACACATGCAAGTCGAGCGAAGCACTTTGCTTTGATTTCTTCGGGATGAAG
AGCTTAGTGACTGAGCGGCGGACGGGTGAGTAACGCGTGGGTAACCTGCC
TCATACAGGGGGATAACAGTTAGAAATGACTGCTAATACCGCATAAGACC
ACAGCACCGCATGGTGCAGGGGTAAAAACTCCGGTGGTATGAGATGGACC
CGCGTCTGATTAGTTAGTTGGTGGGGTAACGGCCTACCAAGGCGACGATC
AGTAGCCGACCTGAGAGGGTGACCGGCCACATTGGGACTGAGACACGGCC
CAAACTCCTACGGGAGGCAGCAGTGGGGAATATTGCACAATGGGGGAAAC
CCTGATGCAGCGACGCCGCGTGAGCGAAGAAGTATTTCGGTATGTAAAGC
TCTATCAGCAGGGAAGAAAATGACGGTACCTGACTAAGAAGCCCCGGCTA
ACTACGTGCCAGCAGCCGCGGTAATACGTAGGGGGCAAGCGTTATCCGGA
TTTACTGGGTGTAAAGGGAGCGTAGACGGAGCAGCAAGTCTGATGTGAAA
ACCCGGGGCTCAACCCCGGGACTGCATTGGAAACTGTTGATCTGGAGTGC
CGGAGAGGTAAGCGGAATTCCTAGTGTAGCGGTGAAATGCGTAGATATTA
GGAGGAACACCAGTGGCGAAGGCGGCTTACTGGACGGTAACTGACGTTGA
GGCTCGAAAGCGTGGGGAGCAAACAGGATTAGATACCCTGGTAGTCCACG
CCGTAAACGATGACTACTAGGTGTCGGGTGGCAAAGCCATTCGGTGCCGC
AGCCAACGCAATAAGTAGTCCACCTGGGGAGTACGTTCGCAAGAATGAAA
CTCAAAGGAATTGACGGGGACCCGCACAAGCGGTGGAGCATGTGGTTTAA
TTCGAAGCAACGCGAAGAACCTTACCTGCTCTTGACATCCCGGTGACGGC
AGAGTAATGTCTGCTTTTCTTCGGAACACCGGTGACAGGTGGTGCATGGT
TGTCGTCAGCTCGTGTCGTGAGATGTTGGGTTAAGTCCCGCAACGAGCGC
AACCCCTATCTTCAGTAGCCAGCGGTAAGGCCGGGCACTCTGGAGAGACT
GCCAGGGATAACCTGGAGGAAGGTGGGGATGACGTCAAATCATCATGCCC
CTTATGAGCAGGGCTACACACGTGCTACAATGGCGTAAACAAAGGGAAGC
GAACCCGCGAGGGTGGGCAAATCCCAAAAATAACGTCTCAGTTCGGATTG
TAGTCTGCAACTCGACTACATGAAGCTGGAATCGCTAGTAATCGCGAATC
AGAATGTCGCGGTGAATACGTTCCCGGGTCTTGTACACACCGCCCGTCAC
ACCATGGGAGTCAGTAACGCCCGAAGTCAGTGACCCAACCGTAAGGAGGG
AGCTGCCGAAGGTGGGACCGATAACTGGGGTGAAGTCGTAACAAGGTAGC
CGTATCGGAAGGTGCGGCTGGATCACCTCCTTTCTAAGGAA

In alternative embodiments, a Blautia obeum species used in formulations as provided herein, or in methods as provided herein, comprises a 16S rDNA sequence having at least about 90%, 95%, 96%, 97% 98% or 99% or complete sequence identity to SEQ ID NO:13:

Blautia obeum 16S ribosomal RNA gene
SEQ ID 13
TTTATCAGAGAGTTTGATCCTGGCTCAGGATGAACGCTGGCGGCGTGCTT
AACACATGCAAGTCGAACGGGAAACTTTTCATTGAAGCTTCGGCAGATTT
GGTCTGTTTCTAGTGGCGGACGGGTGAGTAACGCGTGGGTAACCTGCCTT
ATACAGGGGGATAACAACCAGAAATGGTTGCTAATACCGCATAAGCGCAC
AGGACCGCATGGTCCGGTGTGAAAAACTCCGGTGGTATAAGATGGACCCG
CGTTGGATTAGCTAGTTGGCAGGGTAACGGCCTACCAAGGCGACGATCCA
TAGCCGGCCTGAGAGGGTGAACGGCCACATTGGGACTGAGACACGGCCCA
GACTCCTACGGGAGGCAGCAGTGGGGAATATTGCACAATGGGGGAAACCC
TGATGCAGCGACGCCGCGTGAAGGAAGAAGTATCTCGGTATGTAAACTTC
TATCAGCAGGGAAGATAGTGACGGTACCTGACTAAGAAGCCCCGGCTAAC
TACGTGCCAGCAGCCGCGGTAATACGTAGGGGGCAAGCGTTATCCGGATT
TACTGGGTGTAAAGGGAGCGTAGACGGACTGGCAAGTCTGATGTGAAAGG
CGGGGGCTCAACCCCTGGACTGCATTGGAAACTGTTAGTCTTGAGTGCCG
GAGAGGTAAGCGGAATTCCTAGTGTAGCGGTGAAATGCGTAGATATTAGG
AGGAACACCAGTGGCGAAGGCGGCTTACTGGACGGTAACTGACGTTGAGG
CTCGAAAGCGTGGGGAGCAAACAGGATTAGATACCCTGGTAGTCCACGCC
GTAAACGATGAATACTAGGTGTTGGGGAGCAAAGCTCTTCGGTGCCGCCG
CAAACGCATTAAGTATTCCACCTGGGGAGTACGTTCGCAAGAATGAAACT
CAAAGGAATTGACGGGGACCCGCACAAGCGGTGGAGCATGTGGTTTAATT
CGAAGCAACGCGAAGAACCTTACCAAGTCTTGACATCCCTCTGACCGTTC
CTTAACCGGAACnTCCTTCGGGACAGAGGAGACAGGTGGTGCATGGTTGT
CGTCAGCTCGTGTCGTGAGATGTTGGGTTAAGTCCCGCAACGAGCGCAAC
CCCTATCCCCAGTAGCCAGCGGTTCGGCCGGGCACTCTGAGGAGACTGCC
AGGGATAACCTGGAGGAAGGCGGGGATGACGTCAAATCATCATGCCCCTT
ATGATTTGGGCTACACACGTGCTACAATGGCGTAAACAAAGGGAAGCGAG
CCTGCGAAGGTAAGCAAATCCCAAAAATAACGTCCCAGTTCGGACTGCAG
TCTGCAACTCGACTGCACGAAGCTGGAATCGCTAGTAATCGCGGATCAGA
ATGCCGCGGTGAATACGTTCCCGGGTCTTGTACACACCGCCCGTCACACC
ATGGGAGTCAGTAACGCCCGAAGTCAGTGACCTAACTGCAAAGAAGGAGC
TGCCGAAGGCGGGACCGATGACTGGGGTGAAGTCGTAACAAGGTAGCCGT
ATCGGAAGGTGCGGCTGGATCACCTCCTTTCTAAGGAA

In alternative embodiments, a Coprococcus comes species used in formulations as provided herein, or in methods as provided herein, comprises a 16S rDNA sequence having at least about 90%, 95%, 96%, 97% 98% or 99% or complete sequence identity to SEQ ID NO:14:

Coprococcus comes 16S ribosomal RNA gene
SEQ ID 14
TTTAACGAGAGTTTGATCCTGGCTCAGGATGAACGCTGGCGGCGTGCTTA
ACACATGCAAGTCGAACGAAGCACTTTAACCTGATTCTTCGGATGAAGGT
TTTTGTGACTGAGTGGCGGACGGGTGAGTAACGCGTGGGTAACCTGCCTC
ATACAGGGGGATAACAGTTAGAAATGACTGCTAATACCGCATAAGACCAC
AGAGCCGCATGGCTCGGTGGGAAAAACrCCGGTGGTATGAGATGGACCCG
CGTCTGATTAGGTAGTTGGTGGGGTAACGGCCTACCAAGCCAACGATCAG
TAGCCGACCTGAGAGGGTGACCGGCCACATTGGGACTGAGACACGGCCCA
AACTCCTACGGGAGGCAGCAGTGGGGAATATTGCACAATGGGGGAAACCC
TGATGCAGCGACGCCGCGTGAGCGAAGAAGTATTTCGGTATGTAAAGCTC
TATCAGCAGGGAAGAAAATGACGGTACCTGACTAAGAAGCACCGGCTAAA
TACGTGCCAGCAGCCGCGGTAATACGTATGGTGCAAGCGTTATCCGGATT
TACTGGGTGTAAAGGGAGCGTAGACGGCTGTGTAAGTCTGAAGTGAAAGC
CCGGGGCTCAACCCCGGGACTGCTTTGGAAACTATGCAGCTAGAGTGTCG
GAGAGGTAAGTGGAATTCCCAGTGTAGCGGTGAAATGCGTAGATATTGGG
AGGAACACCAGTGGCGAAGGCGGCTTACTGGACGATGACTGACGTTGAGG
CTCGAAAGCGTGGGGAGCAAACAGGATTAGATACCCTGGTAGTCCACGCC
GTAAACGATGACTACTAGGTGTCGGGGAGCAAAGCTCTTCGGTGCCGCAG
CAAACGCAATAAGTAGTCCACCTGGGGAGTACGTTCGCAAGAATGAAACT
CAAAGGAATTGACGGGGACCCGCACAAGCGGTGGAGCATGTGGTTTAATT
CGAAGCAACGCGAAGAACCTTACCTGCTCTTGACATCCCGGTGACCGGCG
TGTAATGACGCCTTTTCTTCGGAACACCGGTGACAGGTGGTGCATGGTTG
TCGTCAGCTCGTGTCGTGAGATGTTGGGTTAAGTCCCGCAACGAGCGCAA
CCCTTATCTTCAGTAGCCAGCAATTCGGATGGGCACTCTGGAGAGACTGC
CAGGGATAACCTGGAGGAAGGTGGGGATGACGTCAAATCATCATGCCCCT
TATGAGCAGGGCTACACACGTGCTACAATGGCGTAAACAAAGGGAAGCGA
GCCTGCGAGGGTAAGCAAATCTCAAAAATAACGTCTCAGTTCGGATTGTA
GTCTGCAACTCGACTACATGAAGCTGGAATCGCTAGTAATCGCGAATCAG
CATGTCGCGGTGAATACGTTCCCGGGTCTTGTACACACCGCCCGTCACAC
CATGGGAGTTGGTAACGCCCGAAGTCAGTGACCCAACCGTAAGGAGGGAG
CTGCCGAAGGTGGGACCGATAACTGGGGTGAAGTCGTAACAAGGTAGCCG
TATCGGAAGGTGCGGCTGGATCACCTCCTTTCTAAGGAA

In alternative embodiments, a Dorea longicatena species used in formulations as provided herein, or in methods as provided herein, comprises a 16S rDNA sequence having at least about 90%, 95%, 96%, 97% 98% or 99% or complete sequence identity to SEQ ID NO:15:

Dorea longicatena 16S ribosomal RNA gene
SEQ ID 15
TTTAACGAGAGTTTGATCCTGGCTCAGGATGAACGCTGGCGGCGTGCTTA
ACACATGCAAGTCGAGCGAAGCGCTTAAGTTTGATTCTTCGGATGAAGAC
TTTTGTGACTGAGCGGCGGACGGGTGAGTAACGCGTGGGTAACCTGCCTC
ATACAGGGGGATAACAGTTAGAAATGACTGCTAATACCGCATAAGACCAC
GGTACCGCATGGTACAGTGGTAAAAACTCCGGTGGTATGAGATGGACCCG
CGTCTGATTAGGTAGTTGGTGGGGTAACGGCCTACCAAGCCGACGATCAG
TAGCCGACCTGAGAGGGTGACCGGCCACATTGGGACTGAGACACGGCCCA
GACTCCTACGGGAGGCAGCAGTGGGGAATATTGCACAATGGAGGAAACTC
TGATGCAGCGACGCCGCGTGAAGGATGAAGTATTTCGGTATGTAAACTTC
TATCAGCAGGGAAGAAAATGACGGTACCTGACTAAGAAGCCCCGGCTAAC
TACGTGCCAGCAGCCGCGGTAATACGTAGGGGGCAAGCGTTATCCGGATT
TACTGGGTGTAAAGGGAGCGTAGACGGCACGGCAAGCCAGATGTGAAAGC
CCGGGGCTCAACCCCGGGACTGCATTTGGAACTGCTGAGCTAGAGTGTCG
GAGAGGCAAGTGGAATTCCTAGTGTAGCGGTGAAATGCGTAGATATTAGG
AGGAACACCAGTGGCGAAGGCGGCTTGCTGGACGATGACTGACGTTGAGG
CTCGAAAGCGTGGGGAGCAAACAGGATTAGATACCCTGGTAGTCCACGCC
GTAAACGATGACTGCTAGGTGTCGGGTGGCAAAGCCATTCGGTGCCGCAG
CTAACGCAATAAGCAGTCCACCTGGGGAGTACGTTCGCAAGAATGAAACT
CAAAGGAATTGACGGGGACCCGCACAAGCGGTGGAGCATGTGGTTTAATT
CGAAGCAACGCGAAGAACCTTACCTGATCTTGACATCCCGATGACCGCTT
CGTAATGGAAGTTTTTCTTCGGAACATCGGTGACAGGTGGTGCATGGTTG
TCGTCAGCTCGTGTCGTGAGATGTTGGGTTAAGTCCCGCAACGAGCGCAA
CCCCTATCTTCAGTAGCCAGCAGGTTAAGCTGGGCACTCTGGAGAGACTG
CCAGGGATAACCTGGAGGAAGGTGGGGATGACGTCAAATCATCATGCCCC
TTATGACCAGGGCTACACACGTGCTACAATGGCGTAAACAAAGAGAAGCG
AACTCGCGAGGGTAAGCAAATCTCAAAAATAACGTCTCAGTTCGGATTGT
AGTCTGCAACTCGACTACATGAAGCTGGAATCGCTAGTAATCGCAGATCA
GAATGCTGCGGTGAATACGTTCCCGGGTCTTGTACACACCGCCCGTCACA
CCATGGGAGTCAGTAACGCCCGAAGTCAGTGACCCAACCGTAAGGAGGGA
GCTGCCGAAGGTGGGACCGATAACTGGGGTGAAGTCGTAACAAGGTAGCC
GTATCGGAAGGTGCGGCTGGATCACCTCCTTTCTAAGGAA

In alternative embodiments, a Bifidobacterium catenulatum species used in formulations as provided herein, or in methods as provided herein, comprises a 16S rDNA sequence having at least about 90%, 95%, 96%, 97% 98% or 99% or complete sequence identity to SEQ ID NO:16:

Bifidobacterium catenuiatum 16S ribosomal
RNA gene
SEQ ID 16
TTTTGTGGAGGGTTCGATTCTGGCTCAGGATGAACGCrGGCGGCGTGCTT
AACACATGCAAGTCGAACGGGATCCAGGCAGCTTGCTGCCTGGTGAGAGT
GGCGAACGGGTGAGTAATGCGTGACCGACCrGCCCCATACACCGGAATAG
CTCCTGGAAACGGGTGGTAATGCCGGATGCTCCGACTCCTCGCATGGGGT
GTCGGGAAAGATTTCATCGGTATGGGATGGGGTCGCGTCCrATCAGGTAG
TCGGCGGGGTAACGGCCCACCGAGCCTACGACGGGTAGCCGGCCTGAGAG
GGCGACCGGCCACATTGGGACTGAGATACGGCCCAGACTCCTACGGGAGG
CAGCAGTGGGGAATATTGCACAATGGGCGCAAGCCTGATGCAGCGACGCC
GCGTGCGGGATGACGGCCTTCGGGTTGTAAACCGCTTTTGATCGGGAGCA
AGCCTTCGGGTGAGTGTACCTTTCGAATAAGCACCGGCTAACTACGTGCC
AGCAGCCGCGGTAATACGTAGGGTGCAAGCGTTATCCGGAATTATTGGGC
GTAAAGGGCTCGTAGGCGGTTCGTCGCGTCCGGTGTGAAAGTCCATCGCT
TAACGGTGGATCTGCGCCGGGTACGGGCGGGCTGGAGTGCGGTAGGGGAG
ACTGGAATTCCCGGTGTAACGGTGGAATGTGTAGATATCGGGAAGAACAC
CAATGGCGAAGGCAGGTCTCTGGGCCGTTACTGACGCTGAGGAGCGAAAG
CGTGGGGAGCGAACAGGATTAGGAACACCAGTGGCGAAGGCGGCTTACTG
GACGATGACTGACGTTGAGGCTCGAAAGCGTGGGGAGCAAACAGGATTAG
ATACCCTGGTAGTCCACGCCGTAAACGATGACTACTAGGTGTCGGGGAGC
AAAGCTCTTCGGTGCCGCAGCAAACGCAATAAGTAGTCCACCTGGGGAGT
ACGTTCGCAAGAATGAAACTCAAAGGAATTGACGGGGACCCGCACAAGCG
GTGGAGCATGTGGTTTAATTCGAAGCAACGCGAAGAACCTTACCTGCTCT
TGACATCCCGGTGACCGGCGTGTAATGACGCCTTTTCTTCGGAACACCGG
TGACAGGTGGTGCATGGTTGTCGTCAGCTCGTGTCGTGAGATGTTGGGTT
AAGTCCCGCAACGAGCGCAACCCTTATCTTCAGTAGCCAGCAATTCGGAT
GGGCACTCTGGAGAGACTGCCAGGGATAACCTGGAGGAAGGTGGGGATGA
CGTCAAATCATCATGCCCCTTATGAGCAGGGCTACACACGTGCTACAATG
GCGTAAACAAAGGGAAGCGAGCCTGCGAGGGTAAGCAAATCTCAAAAATA
ACGTCTCAGTTCGGATTGTAGTCTGCAACTCGACTACATGAAGCTGGAAT
CGCTAGTAATCGCGAATCAGCATGTCGCGGTGAATACGTTCCCGGGTCTT
GTACACACCGCCCGTCACACCATGGGAGTTGGTAACGCCCGAAGTCAGTG
ACCCAACCGTAAGGAGGGAGCTGCCGAAGGTGGGACCGATAACTGGGGTG
AAGTCGTAACAAGGTAGCCGTATCGGAAGGTGCGGCTGGATCACCTCCTT
TCTAAGGAA

In alternative embodiments, an Akkermansia muciniphila species used in formulations as provided herein, or in methods as provided herein, comprises a 16S rDNA sequence having at least about 90%, 95%, 96%, 97% 98% or 99% or complete sequence identity to SEQ ID NO:17:

Akkermansia muciniphila 16S ribosomal
RNA sequence
SEQ ID 17
ATGGAGAGTTTGATTCTGGCTCAGAACGAACGCTGGCGGCGTGGATAAGA
CATGCAAGTCGAACGAGAGAATTGCTAGCTTGCTAATAATTCTCTAGTGG
CGCACGGGTGAGTAACACGTGAGTAACCTGCCCCCGAGAGCGGGATAGCC
CTGGGAAACTGGGATTAATACCGCATAGTATCGAAAGATTAAAGCAGCAA
TGCGCTTGGGGATGGGCTCGCGGCCTATTAGTTAGTTGGTGAGGTAACGG
CTCACCAAGGCGATGACGGGTAGCCGGTCTGAGAGGATGTCCGGCCACAC
TGGAACTGAGACACGGTCCAGACACCTACGGGTGGCAGCAGTCGAGAATC
ATTCACAATGGGGGAAACCCTGATGGTGCGACGCCGCGTGGGGGAATGAA
GGTCTTCGGATTGTAAACCCCTGTCATGTGGGAGCAAATTAAAAAGATAG
TACCACAAGAGGAAGAGACGGCTAACTCTGTGCCAGCAGCCGCGGTAATA
CAGAGGTCTCAAGCGTTGTTCGGAATCACTGGGCGTAAAGCGTGCGTAGG
CTGTTTCGTAAGTCGTGTGTGAAAGGCGCGGGCTCAACCCGCGGACGGCA
CATGATACTGCGAGACTAGAGTAATGGAGGGGGAACCGGAATTCTCGGTG
TAGCAGTGAAATGCGTAGATATCGAGAGGAACACTCGTGGCGAAGGCGGG
TTCCTGGACATTAACTGACGCTGAGGCACGAAGGCCAGGGGAGCGAAAGG
GATTAGATACCCCTGTAGTCCTGGCAGTAAACGGTGCACGCTTGGTGTGC
GGGGAATCGACCCCCTGCGTGCCGGAGCTAACGCGTTAAGCGTGCCGCCT
GGGGAGTACGGTCGCAAGATTAAAACTCAAAGAAATTGACGGGGACCCGC
ACAAGCGGTGGAGTATGTGGCTTAATTCGATGCAACGCGAAGAACCTTAC
CTGGGCTTGACATGTAATGAACAACATGTGAAAGCATGCGACTCTTCGGA
GGCGTTACACAGGTGCTGCATGGCCGTCGTCAGCTCGTGTCGTGAGATGT
TTGGTTAAGTCCAGCAACGAGCGCAACCCCTGTTGCCAGTTACCAGCACG
TGAAGGTGGGGACTCTGGCGAGACTGCCCAGATCAACTGGGAGGAAGGTG
GGGACGACGTCAGGTCAGTATGGCCCTTATGCCCAGGGCTGCACACGTAC
TACAATGCCCAGTACAGAGGGGGCCGAAGCCGCGAGGCGGAGGAAATCCT
AAAAACTGGGCCCAGTTCGGACTGTAGGCTGCAACCCGCCTACACGAAGC
CGGAATCGCTAGTAATGGCGCATCAGCTACGGCGCCGTGAATACGTTCCC
GGGTCTTGTACACACCGCCCGTCACATCATGGAAGCCGGTCGCACCCGAA
GTATCTGAAGCCAACCGCAAGGAGGCAGGGTCCTAAGGTGAGACTGGTAA
CTGGGATGAAGTCGTAACAAGGTAGCCGTAGGGGAACCTGCGGCTGGATC
ACCTCCTTTCTATGGA

In alternative embodiments, a Ruminococcus gnavus species used in formulations as provided herein, or in methods as provided herein, comprises a 16S rDNA sequence having at least about 90%, 95%, 96%, 97% 98% or 99% or complete sequence identity to SEQ ID NO:18:

Ruminococcus gnavus 16S ribosomal RNA gene
SEQ ID 18
TTTAACGAGAGTTTGATCCTGGCTCAGGATGAACGCTGGCGGCGTGCTTA
ACACATGCAAGTCGAGCGAAGCACCTTGACGGATTTCTTCGGATTGAAGC
CTTGGTGACTGAGCGGCGGACGGGTGAGTAACGCGTGGGTAACCTGCCTC
GTACAGGGGGATAACAGTTGGAAACGGCTGCTAATACCGCATAAGCGCAC
AGTACCGCATGGTACGGTGTGAAAAACTCCGGTGGTATGAGATGGACCCG
CGTCTGATTAGGTAGTTGGTGGGGTAACGGCCTACCAAGCCGACGATCAG
TAGCCGACCTGAGAGGGTGACCGGCCACATTGGGACTGAGACACGGCCCA
AACTCCTACGGGAGGCAGCAGTGGGGAATATTGCACAATGGGGGAAACCC
TGATGCAGCGACGCCGCGTGAGCGATGAAGTATTTCGGTATGTAAAGCTC
TATCAGCAGGGAAGAAAATGACGGTACCTGACTAAGAAGCCCCGGCTAAC
TACGTGCCAGCAGCCGCGGTAATACGTAGGGGGCAAGCGTTATCCGGATT
TACTGGGTGTAAAGGGAGCGTAGACGGCATGGCAAGCCAGATGTGAAAGC
CCGGGGCTCAACCCCGGGACTGCATTTGGAACTGTCAGGCTAGAGTGTCG
GAGAGGAAAGCGGAATTCCTAGTGTAGCGGTGAAATGCGTAGATATTAGG
AGGAACACCAGTGGCGAAGGCGGCTTTCTGGACGATGACTGACGTTGAGG
CTCGAAAGCGTGGGGAGCAAACAGGATTAGATACCCTGGTAGTCCACGCC
GTAAACGATGAATACTAGGTGTCGGGTGGCAAAGCCATTCGGTGCCGCAG
CAAACGCAATAAGTATTCCACCTGGGGAGTACGTTCGCAAGAATGAAACT
CAAAGGAATTGACGGGGACCCGCACAAGCGGTGGAGCATGTGGTTTAATT
CGAAGCAACGCGAAGAACCTTACCTGGTCTTGACATCCCTCTGACCGCTC
TTTAATCGGAGCTTTCCTTCGGGACAGAGGAGACAGGTGGTGCATGGTTG
TCGTCAGCTCGTGTCGTGAGATGTTGGGTTAAGTCCCGCAACGAGCGCAA
CCCCTATCTTTACTAGCCAGCATTTTGGATGGGCACTCTAGAGAGACTGC
CAGGGATAACCTGGAGGAAGGTGGGGATGACGTCAAATCATCATGCCCCT
TATGACCAGGGCTACACACGTGCTACAATGGCGTAAACAAAGGGAAGCGA
GCCCGCGAGGGGGAGCAAATCCCAAAAATAACGTCTCAGTTCGGATTGTA
GTCTGCAACTCGACTACATGAAGCTGGAATCGCTAGTAATCGCGAATCAG
AATGTCGCGGTGAATACGTTCCCGGGTCTTGTACACACCGCCCGTCACAC
CATGGGAGTCAGTAACGCCCGAAGTCAGTGACCCAACCGCAAGGAGGGAG
CTGCCGAAGGTGGGACCGATAACTGGGGTGAAGTCGTAACAAGGTAGCCG
TATCGGAAGGTGCGGCTGGATCACCTCCTTTCTAAGGA

In alternative embodiments, a Ruminococcus torques species used in formulations as provided herein, or in methods as provided herein, comprises a 16S rDNA sequence having at least about 90%, 95%, 96%, 97% 98% or 99% or complete sequence identity to SEQ ID NO: 19:

Ruminococcus torques 16S ribosomal RNA gene
SEQ ID 19
TTTAACGAGAGTTTGATCCTGGCTCAGGATGAACGCTGGCGGCGTGCCTA
ACACATGCAAGTCGAGCGAAGCACTTTGCTTAGATTCTTCGGATGAAGAG
GATTGTGACTGAGCGGCGGACGGGTGAGTAACGCGTGGGTAACCTGCCTC
ATACAGGGGGATAACAGTTAGAAATGACTGCTAATACCGCATAAGACCAC
AGCACCGCATGGTGCGGGGGTAAAAACTCCGGTGGTATGAGATGGACCCG
CGTCTGATTAGCTAGTTGGTAAGGTAACGGCTTACCAAGGCGACGATCAG
TAGCCGACCTGAGAGGGTGGCCGGCCACATTGGGACTGAGACACGGCCCA
AACTCCTACGGGAGGCAGCAGTGGGGAATATTGCACAATGGGGGAAACCC
TGATGCAGCGACGCCGCGTGAGCGAAGAAGTATTTCGGTATGTAAAGCTC
TATCAGCAGGGAAGAAAATGACGGTACCTGACTAAGAAGCACCGGCTAAA
TACGTGCCAGCAGCCGCGGTAATACGTATGGTGCAAGCGTTATCCGGATT
TACTGGGTGTAAAGGGAGCGTAGACGGATGGGCAAGTCTGATGTGAAAAC
CCGGGGCTCAACCCCGGGACTGCATTGGAAACTGTTCATCTAGAGTGCTG
GAGAGGTAAGTGGAATTCCTAGTGTAGCGGTGAAATGCGTAGATATTAGG
AGGAACACCAGTGGCGAAGGCGGCTTACTGGACAGTAACTGACGTTGAGG
CTCGAAAGCGTGGGGAGCAAACAGGATTAGATACCCTGGTAGTCCACGCC
GTAAACGATGACTACTAGGTGTCGGGTGGCAAAGCCATTCGGTGCCGCAG
CAAACGCAATAAGTAGTCCACCTGGGGAGTACGTTCGCAAGAATGAAACT
CAAAGGAATTGACGGGGACCCGCACAAGCGGTGGAGCATGTGGTTTAATT
CGAAGCAACGCGAAGAACCTTACCTGCTCTTGACATCCCGCTGACCGGAC
GGTAATGCGTCCTTCCCTTCGGGGCAGCGGAGACAGGTGGTGCATGGTTG
TCGTCAGCTCGTGTCGTGAGATGTTGGGTTAAGTCCCGCAACGAGCGCAA
CCCCTATCTTTAGTAGCCAGCGGCCAGGCCGGGCACTCTAGAGAGACTGC
CGGGGATAACCCGGAGGAAGGTGGGGATGACGTCAAATCATCATGCCCCT
TATGAGCAGGGCTACACACGTGCTACAATGGCGTAAACAAAGGGAAGCGA
GACCGCGAGGTGGAGCAAATCCCAAAAATAACGTCTCAGTTCGGATTGTA
GTCTGCAACTCGACTACATGAAGCTGGAATCGCTAGTAATCGCGAATCAG
AATGTCGCGGTGAATACGTTCCCGGGTCTTGTACACACCGCCCGTCACAC
CATGGGAGTCAGTAACGCCCGAAGTCAGTGACCCAACCGTAAGGAGGGAG
CTGCCGAAGGCGGGACCGATAACTGGGGGTGAAGTCGTAACAAGGTAGCC
GTATCGGAAGGTGCGGCTGGATCACCTCCTTTCTAAGGAA

In alternative embodiments, a Clostridium scindens species used in formulations as provided herein, or in methods as provided herein, comprises a 16S rDNA sequence having at least about 90%, 95%, 96%, 97% 98% or 99% or complete sequence identity to SEQ ID NO:20:

Clostridium scindens 16S ribosomal RNA gene
SEQ ID 20
GAGAGTTTGATCCTGGCTCAGGATGAACGCTGGCGGCGTGCCTAACACAT
GCAAGTCGAACGAAGCGCCTGGCCCCGACTTCTTCGGAACGAGGAGCCTT
GCGACTGAGTGGCGGACGGGTGAGTAACGCGTGGGCAACCTGCCTTGCAC
TGGGGGATAACAGCCAGAAATGGCTGCTAATACCGCATAAGACCGAAGCG
CCGCATGGCGCGGCGGCCAAAGCCCCGGCGGTGCAAGATGGGCCCGCGTC
TGATTAGGTAGTTGGCGGGGTAACGGCCCACCAAGCCGACGATCAGTAGC
CGACCTGAGAGGGTGACCGGCCACATTGGGACTGAGACACGGCCCAGACT
CCTACGGGAGGCAGCAGTGGGGAATATTGCACAATGGGGGAAACCCTGAT
GCAGCGACGCCGCGTGAAGGATGAAGTATTTCGGTATGTAAACTTCTATC
AGCAGGGAAGAAGATGACGGTACCTGACTAAGAAGCCCCGGCTAACTACG
TGCCAGCAGCCGCGGTAATACGTAGGGGGCAAGCGTTATCCGGATTTACT
GGGTGTAAAGGGAGCGTAGACGGCGATGCAAGCCAGATGTGAAAGCCCGG
GGCTCAACCCCGGGACTGCATTTGGAACTGCGTGGCTGGAGTGTCGGAGA
GGCAGGCGGAATTCCTAGTGTAGCGGTGAAATGCGTAGATATTAGGAGGA
ACACCAGTGGCGAAGGCGGCCTGCTGGACGATGACTGACGTTGAGGCTCG
AAAGCGTGGGGAGCAAACAGGATTAGATACCCTGGTAGTCCACGCCGTAA
ACGATGACTACTAGGTGTCGGGTGGCAAGGCCATTCGGTGCCGCAGCAAA
CGCAATAAGTAGTCCACCTGGGGAGTACGTTCGCAAGAATGAAACTCAAA
GGAATTGACGGGGACCCGCACAAGCGGTGGAGCATGTGGTTTAATTCGAA
GCAACGCGAAGAACCTTACCTGATCTTGACATCCCGATGCCAAAGCGCGT
AACGCGCTCTTTCTTCGGAACATCGGTGACAGGTGGTGCATGGTTGTCGT
CAGCTCGTGTCGTGAGATGTTGGGTTAAGTCCCGCAACGAGCGCAACCCC
TATCTTCAGTAGCCAGCATTTTGGATGGGCACTCTGGAGAGACTGCCAGG
GAGAACCTGGAGGAAGGTGGGGATGACGTCAAATCATCATGCCCCTTATG
ACCAGGGCTACACACGTGCTACAATGGCGTAAACAAAGGGAGGCGAACCC
GCGAGGGTGGGCAAATCCCAAAAATAACGTGTCAGTTCGGATTGTAGTCT
GCAACTCGACTACATGAAGTTGGAATCGCTAGTAATCGCGAATCAGAATG
TCGCGGTGAATACGTTCCCGGGTCTTGTACACACCGCCCGTCACACCATG
GGAGTCAGTAACGCCCGAAGCCGGTGACCCAACCCGTAAGGGAGGGAGCC
GTCGAAGGTGGGACCGATAACTGGGGTGAAGTCGTAACAAGGTAGCCGTA
TCGGAAGGTGCGGCTGGATCACCTCCTTC

In alternative embodiments, a Enterococcus hirae species used in formulations as provided herein, or in methods as provided herein, comprises a 16S rDNA sequence having at least about 90%, 95%, 96%, 97% 98% or 99% or complete sequence identity to SEQ ID NO:21:

Enterococcus hirae 16S ribosomal RNA gene
SEQ ID 21
TGAGAGTTTGATCCTGGCTCAGGACGAACGCTGGCGGCGTGCCTAATACA
TGCAAGTCGAACGCTTCTTTTTCCACCGGAGCTTGCTCCACCGGAAAAAG
AGGAGTGGCGAACGGGTGAGTAACACGTGGGTAACCTGCCCATCAGAAGG
GGATAACACTTGGAAACAGGTGCTAATACCGTATAACAATCGAAACCGCA
TGGTTTTGATTTGAAAGGCGCTTTCGGGTGTCGCTGATGGATGGACCCGC
GGTGCATTAGCTAGTTGGTGAGGTAACGGCTCACCAAGGCGACGATGCAT
AGCCGACCTGAGAGGGTGATCGGCCACATTGGGACTGAGACACGGCCCAA
ACTCCTACGGGAGGCAGCAGTAGGGAATCTTCGGCAATGGACGAAAGTCT
GACCGAGCAACGCCGCGTGAGTGAAGAAGGTTTTCGGATCGTAAAACTCT
GTTGTTAGAGAAGAACAAGGATGAGAGTAACTGTTCATCCCTTGACGGTA
TCTAACCAGAAAGCCACGGCTAACTACGTGCCAGCAGCCGCGGTAATACG
TAGGTGGCAAGCGTTGTCCGGATTTATTGGGCGTAAAGCGAGCGCAGGCG
GTTTCTTAAGTCTGATGTGAAAGCCCCCGGCTCAACCGGGGAGGGTCATT
GGAAACTGGGAGACTTGAGTGCAGAAGAGGAGAGTGGAATTCCATGTGTA
GCGGTGAAATGCGTAGATATATGGAGGAACACCAGTGGCGAAGGCGGCTC
TCTGGTCTGTAACTGACGCTGAGGCTCGAAAGCGTGGGGAGCAAACAGGA
TTAGATACCCTGGTAGTCCACGCCGTAAACGATGAGTGCTAAGTGTTGGA
GGGTTTCCGCCCTTCAGTGCTGCAGCTAACGCATTAAGCACTCCGCCTGG
GGAGTACGACCGCAAGGTTGAAACTCAAAGGAATTGACGGGGGCCCGCAC
AAGCGGTGGAGCATGTGGTTTAATTCGAAGCAACGCGAAGAACCTTACCA
GGTCTTGACATCCTTTGACCACTCTAGAGATAGAGCTTCCCCTTCGGGGG
CAAAGTGACAGGTGGTGCATGGTTGTCGTCAGCTCGTGTCGTGAGATGTT
GGGTTAAGTCCCGCAACGAGCGCAACCCTTATTGTTAGTTGCCATCATTT
AGTTGGGCACTCTAGCAAGACTGCCGGTGACAAACCGGAGGAAGGTGGGG
ATGACGTCAAATCATCATGCCCCTTATGACCTGGGCTACACACGTGCTAC
AATGGGAAGTACAACGAGTCGCAAAGTCGCGAGGCTAAGCTAATCTCTTA
AAGCTTCrCTCAGTTCGGATTGTAGGCTGCAACTCGCCTACATGAAGCCG
GAATCGCTAGTAATCGCGGATCAGCACGCCGCGGTGAATACGTTCCCGGG
CCTTGTACACACCGCCCGTCACACCACGAGAGTTTGTAACACCCGAAGTC
GGTGAGGTAACCTTTTGGAGCCAGCCGCCTAAGGTGGGATAGATGATTGG
GGTGAAGTCGTAACAAGGTAGCCGTATCGGAAGGTGCGGCTGGATCACCT
CCTTTCTAAGGAA

In alternative embodiments, bacterial strains used in formulations as provided herein, or in methods as provided herein, are identified by their sequence identity to the variable domains of 16S rDNA.

For example, in alternative embodiments, a species used in formulations as provided herein, or in methods as provided herein, comprises a 16S rDNA sequence having at least about 90%, 95%, 96%, 97% 98% or 99% or complete sequence identity to the variable portion of a particular 16S rDNA sequence, wherein the variable portion of the 16S rDNA is: Nucleotides 137-242; Nucleotides 433-497; and/or Nucleotides 986-1043, of the 16S rDNA sequence.

For example, in alternative embodiments, a Enterococcus hirae species used in formulations as provided herein, or in methods as provided herein, comprises a 16S rDNA sequence having at least about 90%, 95%, 96%, 97% 98% or 99% or complete sequence identity to the variable portions of the 16S rDNA: Nucleotides 137-242; Nucleotides 433-497; and/or Nucleotides 986-1043, of SEQ ID NO:21:

In alternative embodiments, for sequence comparison, one sequence acts as a reference sequence, to which another sequence is compared. Methods of alignment of sequences for comparison are well known in the art. See, for example, by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman Proc. Natl. Acad. Sci. USA (1998) 85:2444, by computerized implementations of these algorithms (GAP, BESTFIT, FASTA, and TFASTA in the Wisconsin Genetics Software Package, Genetics Computer Group. Madison. Wis.), or by manual alignment and visual inspection (see. for example, Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. (1977) 25:3389-3402, and Altschul et al., J. Mol. Biol. (1990) 215:403-410, respectively.

In alternative embodiments, align methods comprise use of a BLAST™ analysis employing: (i) a scoring matrix (such as, e.g., BLOSSUM 62™ or PAM 120™) to assign a weighted homology value to each residue and (ii) a filtering program(s) (such as SEG™ or XNU™) that recognizes and eliminates highly repeated sequences from the calculation. In alternative embodiments, align methods comprise use of a BLAST™ analysis employing a BLAST version 2.2.2 algorithm where a filtering setting is set to blastall -p blastp -d “nr pataa”-F F, and all other options are set to default.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

The following Examples describe methods and compositions for practicing embodiments as provided herein, including methods for making and using compositions comprising non-pathogenic bacteria and non-pathogenic germinable bacterial spores used to practice methods as provide herein.

### Example 1: Anaerobic Culture Conditions

### Preparation of Anaerobic Growth Medium

Exemplary bacterial strains described herein are obligate anaerobes that require anaerobic conditions for culture. Growth media suitable for culture of anaerobic bacteria include reducing agents such as L-cysteine, sodium thioglycolate, and dithiothreitol, for the purpose of scavenging and removing oxygen. Appropriate commercially available anaerobic growth media include but are not limited to Anaerobe Basal Broth (Oxoid/Thermo Scientific), Reinforced Clostridial Medium (Oxoid/Thermo Scientific), Wilkins-Chalgren Anaerobe Broth (Oxoid/Thermo Scientific), Schaedler Anaerobe Broth (Oxoid/Thermo Scientific), and Brain Heart Infusion Broth (Oxoid/Thermo Scientific). Animal free medium for anaerobic culture include but are not limited to Vegitone Actinomyces Broth (Millipore-Sigma), MRS Broth (Millipore-Sigma), Vegitone Infusion Broth (Millipore-Sigma), and Vegitone Casein Soya Broth (Millipore-Sigma).

One liter of Anaerobic growth medium is prepared by combining the manufacturer's recommended amount in grams of dry growth medium powder with 800 ml Reagent Grade Water (NERL™) along with 1 ml 2.5 mg/ml resazurin (ACROS Organics™) in a 2 liter beaker and stirred on a heated stir plate until dissolved. The volume is adjusted to 1 liter by addition of additional Reagent Grade Water, then the volume is brought to a boil while stirring until the red color imbued by the resazurin becomes colorless, indicating removal of oxygen from the solution. The volume is then removed from the stir plate to cool for 10 minutes on the benchtop before further manipulation.

From the 1-liter volume, 900 ml is transferred to a 1 liter anaerobic media bottle (Chemglass Life Sciences) and then placed back on the heated stir plate to remove any oxygen introduced in the transfer, as indicated by the color of the added resazurin. The anaerobic media bottle is then stoppered with a butyl rubber bung that is secured by a crimped aluminum collar, and then brought into the anaerobic chamber (Coy Lab Type A Vinyl Anaerobic Chamber, Coy Laboratory Products, Grass Lake, Mich.). The butyl rubber bung is removed to open the bottle within the anaerobic chamber to equilibrate with the anoxic atmosphere while cooling to ambient temperature. The bottle is resealed with a fresh butyl rubber bung and crimped aluminum collar, brought out of the chamber, then sterilized by autoclaving for 20 minutes followed by slow exhaust.

Alternatively, the 1-liter volume can be aliquoted into smaller 50 ml volumes in 100 ml serum bottles (Chemglass Life Sciences, Vineland N.J.). The boiled 1-liter volume is transferred to a one-liter screwcap bottle, which is placed back on the heated stir plate to drive off any oxygen introduced by the transfer. The bottle cap is then securely tightened, and the bottle is immediately brought into the anaerobic chamber, where the cap is loosened to allow the volume to equilibrate with the anoxic atmosphere and to cool for 1 hour. The volume is then transferred in 50 ml aliquots to 100 ml serum bottles using a serological pipette, then the liquid contents cooled to ambient temperature. The bottles are sealed with butyl rubber bungs and crimped aluminum collars, brought out of the chamber, then sterilized by autoclaving for 20 minutes followed by slow exhaust.

Alternatively, the 1-liter volume can be aliquoted into smaller 10 ml volumes in sealed Hungate tubes (Chemglass Life Sciences, Vineland N.J.) as follows. The boiled 1-liter volume is transferred to a one-liter screwcap bottle, which is placed back on the heated stir plate to drive off any oxygen introduced by the transfer. The bottle cap is then securely tightened, and the bottle is immediately brought into the anaerobic chamber, where the cap is loosened to allow the volume to equilibrate with the anoxic atmosphere and to cool for 1 hour. The volume is then transferred in 10 ml aliquots to fill racked Hungate tubes, then allowed to cool to ambient temperature, followed by securely capping and sealing each tube with screwcaps with butyl rubber septa. The sealed Hungate tube aliquots are removed from the anaerobic chamber and then sterilized by autoclaving for 20 minutes followed by slow exhaust.

Alternatively, the 1 liter volume can be combined with 15 grams Agar (Thermo Scientific™) to make solid media in culture plates as follows: The boiled 1 liter volume is poured into a 1 liter screwcap bottle, followed by replacement on a heated stir plate to remove any oxygen introduced by the transfer as indicated by the colorless resazurin oxygen indicator. The bottle is loosely capped and then autoclaved for 20 minutes followed by slow exhaust. Immediately after autoclaving, the cap of the bottle is tightened prior to bringing the bottle into the anaerobic chamber. Once in the anaerobic chamber, the cap is loosened and the contents cooled for 30 minutes, then 25 ml volumes are poured into culture plates and allowed to cool until solidified. The plates are then allowed to dry in the anaerobic chamber for 24 hours prior to use.

### Live Cryostorage of Anaerobic Microbes

Individual microbes of interest are prepared for long-term cryogenic live storage by inoculating a pure colony isolate grown on anaerobic solid medium into a prepared Hungate tube containing liquid anaerobic growth medium previously determined to be optimal for the species. The inoculated Hungate tube is then incubated at 37° C. until turbidity evident of exponential growth is observed. The Hungate culture is brought into the anaerobic chamber, and 1 ml is transferred by pipette into a 2 ml screwcap cryotube containing anoxic 1 ml Biobank Buffer (Phosphate Buffered Saline (PBS) plus 2% trehalose plus 10% dimethyl sulfoxide, filter sterilized and bubbled with nitrogen gas to remove oxygen). The resulting 2 ml volume is thoroughly mixed by pipetting, securely tightened, then placed for long-term storage in the gaseous phase of a liquid nitrogen Dewar or in a −80° C. freezer.

Microbes in fecal matter can be cryogenically preserved for later revival and new strain discovery as follows. Freshly obtained fecal material is brought into the anaerobic chamber and 1 gram is weighed and mixed in a 15 ml conical tube with a solution consisting of 5 ml Anaerobe Basal Broth (ABB) and 5 ml Biobank Buffer. The tube is tightly capped, and the fecal matter is thoroughly suspended in the solution by vortexing for 20 minutes, followed by incubation upright on ice to allow large particles to settle. One ml aliquots of the fecal suspension are then transferred by pipette to a 2 ml screwcap cryotube, securely tightened, then placed for long-term storage in the gaseous phase of a liquid nitrogen Dewar or in a −80° C. freezer.

### Example 2: Fecal Matter Collection from Patients and Processing

Fecal matter donations are acquired from healthy volunteers as well as individuals exhibiting disease symptoms. Donors can be patients being administered approved anti-viral therapies or participating in clinical trials testing various anti-viral drug or treatment regimens. Donors can be healthy volunteers that do not exhibit disease symptoms.

Donors receive a stool sampling kit by mail sent to the contact address provided or by their physician. Stool samples are collected by the subject at home, or with necessary assistance if hospitalized. Stool sampling kits consist of the following: gloves, instructions for stool collection, welcome card, freezer pack, Styrofoam container, plastic bracket and plastic commode to aid in stool collection, Bristol stool chart, FedEx shipping labels, and stickers to seal kit prior to shipping. Subjects receive a freezer pack for chilling the samples and are instructed to place it in their freezer overnight upon receipt of the sampling kit. The stool sampling kit also includes a plastic commode that can be placed safely and securely on a toilet seat, allowing the subject to defecate directly into a plastic container. The subject is instructed to use the commode to capture a stool sample, then seal the sample container with a provided snap-cap lid. Subjects are instructed to wear the gloves provided in the kit before removing the sample container from the toilet. The subject is instructed to seal the plastic container inside a specimen bag and remove gloves. The subject is then instructed to remove the ice pack from their home freezer and place it inside the Styrofoam cooler box along with the bagged and sealed stool sample, and the graded Bristol Stool card (form indicating stool collection date/time and consistency). The subject is instructed to close the lid on the foam container and then close the box, sealing with the packing sticker. The subject is instructed to schedule a FedEx pickup at their home within 24 hours of stool collection or drop it off at the nearest FedEx location. Under these conditions the stool has been demonstrated to remain chilled during shipment for as long as 48 hours.

Once received, the stool sample receptacle is given a unique alphanumeric identifier that is used subsequently for sample tracking. The stool is unpacked from the shipping box in a laboratory setting, homogenized, and divided into enough individual aliquots for all projected analyses prior to freezing and storage at −80° C., as described below. All aliquots also bear an alphanumeric identifier corresponding to the subject donor. Any remaining stool after the aliquots are taken is disposed as biohazardous waste.

### Preparation of Fecal Matter Samples for Analysis

Fecal matter received from donors can be processed using any method known in the art, for example, as described in U.S. Pat. Nos. 10,493,111; 10,471,107; 10,286,012; 10,314,863; 9,623,056.

For example, received fecal matter in its receptacle is placed on ice and then brought into the anaerobic chamber. The receptacle is opened and approximately 40 g stool is weighed into a tared specimen cup. 15 ml sterile anoxic PBS is then added, and the mixture is homogenized by a hand-held homogenizer to achieve a smooth consistency.

The homogenized fecal matter is then processed and aliquoted for cryo-preservation for several different analyses as follows:
1) For Genomic and Transcriptomic Analyses: homogenized fecal matter is weighed and then an equal volume to weight amount of RNAlater® (Thermo Fisher Scientific) solution is added. The tube is capped tightly and then vortexed for 20 seconds and then placed on ice. A pipette is used to transfer 1 ml aliquots into 2 ml Eppendorf tubes. Aliquoted samples are frozen on dry ice and then stored at −80° C.
2) Live Cryopreservation for Fecal Microbiome Transfer (FMT) Experiments in Mice: Homogenized fecal matter is combined with FMT Buffer (Phosphate Buffered Saline plus 1% L-Cysteine plus 2% Trehalose plus 30% glycerol). The tube is then vortexed for 20 seconds and then placed on ice. A pipette is used to transfer 1 ml aliquots into 2 ml cryotubes that are then tightly capped. Aliquoted samples are frozen on dry ice and then stored at −80° C.
3) Live Cryopreservation for Isolation and Discovery of Microbes: Homogenized fecal matter is combined in a conical tube with Anaerobe Basal Broth and Biobank Buffer (Phosphate Buffered Saline plus 2% Trehalose plus 10% dimethyl sulfoxide), tightly capped and vortexed for 20 seconds, then put on ice upright and allowed to settle for 10 minutes. Using a pipette, 1 ml aliquots are added to 2 ml cryotubes, which are then tightly capped. Aliquoted samples are frozen on dry ice and then stored at −80° C.
   For Genomic and Metabolomic Analyses: Homogenized fecal matter is added to a plastic bag. About 1 cm of the tip end of the bag is cut off with scissors, then aliquots are made by manually squeezing 1 ml of the bag contents into 2 ml Eppendorf tubes. Aliquoted samples are frozen on dry ice and then stored at −80° C.

### Example 3: Isolation and Characterization of Pure Microbial Strains from Fecal Matter

In alternative embodiments, microbes used in compositions as provided herein, or used to practice methods as provided herein, are isolated from fecal matter, and can be used on the form of a pure microbial strain isolated from fecal matter.

Individual bacterial strains can be isolated and cultured from fecal matter material for further study and for assembly of therapeutic biologicals, i.e. for manufacturing combinations of microbes as provided herein. The majority of live bacteria that inhabit fecal matter tend to be obligate anaerobes so care must be taken to perform all culture and isolation work in the anaerobic chamber to prevent their exposure to oxygen, and to use various anaerobic growth media that includes reductant compounds as described in Example 1. Growth media that favor growth of target bacteria can be used to improve the ability to find and isolate them as pure living cultures. Different anaerobic growth media are used to enable growth of different subsets of microbes to improve overall ability to isolate and purify an inclusive number of unique bacterial species from each individual fecal material sample.

To begin a microbial isolation and characterization campaign, one cryotube containing cryogenically preserved fecal matter is removed from storage in the liquid nitrogen Dewar, brought into the anaerobic chamber, and then allowed to thaw gently on ice. The entire 1 ml contents are added to 10 ml of Anaerobe Basal Broth (ABB) or another suitable anaerobic growth medium to establish a 1/10 dilution. Successive 10-fold serial dilutions are then performed in ABB to establish 1/100, 1/1000, 1/10000, 1/100000, 1/1000000 dilutions of the fecal matter. From each of the 1/10000, 1/100000, and 1,1000000 dilutions, four 0.1 ml volumes are removed and then added to and spread over solid anaerobic growth medium of choice. The platings are incubated at 37° C. for 1, 2, 3, 4, 5, 6, 7, 8, 9, 10, 11, 12 days to allow for a wide variety of bacterial colonies to grow. Platings are made from several liquid dilutions of fecal matter to ensure that there will be ones that have numerous yet non-overlapping colonies for efficient colony picking.

Colonies are manually picked from plates using sterile pipette tips. Colonies may also be picked by an automated colony picking machine that is enclosed in an anaerobic chamber. Colonies are picked in multiples of 96 to accommodate subsequent 96-well-based genomic DNA isolation steps and large-scale cryogenic storage steps. The individual picked colonies are then struck on solid anaerobic growth medium of choice to isolate single purified colonies from each picked colony, and then incubated at 37° C. for 1, 2, 3, 4, 5, 6, 7, 8, 9, 10, 11, 12 days to allow for visible colony growth to arise. After visible colonies are evident on the streak, single colonies are picked and then each inoculated into an individual well of a 2 ml 96-well deep well block, each well with 1 ml liquid anaerobic growth medium of choice. Once all wells of the deep-well block have been inoculated with different picked colonies, the deep well block is covered with an adhesive gas-permeable seal and then incubated at 37° C. in an incubator within the anaerobic chamber for 1, 2, 3, 4, 5, 6, 7, 8, 9, 10, 11, 12 days to allow for liquid growth from each isolated colony.

After turbid growth is apparent in all wells, the gas-permeable seal is removed from the 96-well deep well block and a viable stock representation is made by transferring 0.1 ml culture from each well to the corresponding wells of a second 96-well deep-well block, each well containing 0.4 ml of the same anaerobic growth medium plus 0.5 ml Biobank Buffer (Phosphate Buffered Saline plus 2% Trehalose plus 10% dimethyl sulfoxide. The volumes in each well are thoroughly mixed by pipetting up and down several times, then the deep-well block is sealed with an impermeable foil seal rated for −80° C. storage, then stored in a −80° C. freezer.

### Sequence and Computational Characterization of Isolated Fecal Bacteria

The remaining 0.9 ml culture in the original 96-well deep-well plate is then used for whole genome sequence determination of the isolated strain as follows: The deep-well block is subjected to centrifugation for 20 minutes at 6000 g to pellet the cells. After centrifugation, 0.8 ml supernatant is carefully removed by pipette, leaving 0.1 ml pellet and medium for gDNA processing. Total genomic DNA is extracted from the cell pellet using the MagAttract PowerMicrobiome DNA/RNA EP kit (Qiagen). Genomic DNA is then prepared for Whole Genome Sequencing analysis using the sparQ DNA Frag & Library Prep kit (Quantabio). Sequencing analysis is conducted on the Illumina platform using paired-end 150 bp reads.

Sequencing data is processed to remove low quality reads and adapter contamination using Trim Galore, a wrapper for cutadapt (https://journal.embnet.org/index.php/embnetjournal/article/view/200).

The high-quality reads for each isolate are compared against each bacterial or archaeal assembly in NCBI RefSeq using mash (https://genomebiology.biomedcentral.com/articles/10.1186/s13059-016-0997-x). This identifies the most similar organism in the RefSeq database to each isolate at the species and strain level. If the distance reported by mash is below 0.01, the isolate is assumed to be the same strain as the reference strain. If the distance is less than 0.04, the isolate is assumed to be of the same species as the reference strain. If the distance is greater than 0.04, the isolate is assumed to be of a potentially novel species; these isolates are handled on a case-by-case basis.

Further analysis is performed on isolates of interest by assembling with SPAdes (https://www.ncbi.nlm.nih.gov/pmc/articles/PMC3342519/) and using mummer (https://journals.plos.org/ploscompbiol/article?id=10.1371/journal.pcbi.1005944) to align the reference genome and isolate genome against each other.

Complete genomes are generated for organisms of special interest using long-read sequencing. High molecular weight genomic DNA is prepared from organisms of interest using a commercially available kit for example GENOMIC-TIP™ (Qiagen). Library preparation on genomic DNA is performed using the Ligation Sequencing Kit (Oxford Nanopore) and sequencing is performed on a MINION™ (MinION™) (Oxford Nanopore). Reads are filtered and trimmed for quality and assembly is performed using the assembler Flye (Kolmogorov et al. (2019) Nature Biotechnology 37:540-546). The resulting assembly is polished using multiple rounds of pilon (Walker et al. (2014) PLOS ONE 9:e112963) with short reads to correct for errors inherent in long read sequencing. Genes are predicted on the polished genome using prodigal (Hyatt et al. (2010) BMC Bioinformatics 11:119) or the NCBI Prokaryotic Gene Annotation Pipeline (Tatusova et al. (2016) Nucleic Acids Research 44(14):6614-24). Results of this analysis on isolates collected so far are provided in Table 1.

### Identification of Viral Genetic Material in Stool or Blood Samples

Several approaches well known in the art are used to determine the presence or identity of infective viral material in the stool and blood of patients. For example, specific DNA viruses of interest are detected by PCR or real time PCR (RT-PCR) using primers specific to the virus of interest. An analogous procedure is used for RNA viruses, with reverse transcription followed by RT-PCR. Alternatively, DNA or RNA is extracted from the whole stool or blood sample and sequenced by whole genome sequencing. Total genomic DNA is extracted from the stool using the MagAttract PowerMicrobiome DNA/RNA EP kit (Qiagen), and from blood using the QIAamp DNA Blood Mini Kit (Qiagen). Genomic DNA is then prepared for Whole Genome Sequencing analysis using the sparQ DNA Frag & Library Prep kit (Quantabio). RNA is extracted from the stool or blood sample by binding to an RNeasy™ column (Qiagen) followed by washing and elution using the reagents provided in the RNeasy™ kit (Qiagen). Sequencing libraries are prepared from RNA by fragmentation, ribodepletion, cDNA synthesis, PCR amplification, and barcoding as described in the TRUSEQ® mRNA sample preparation kit (Illumina). Sequencing analysis is conducted on the Illumina platform using paired-end 150 bp reads. Reads not mapping to human or bacterial DNA are then aligned to a viral sequence database, for example the NCBI viral genomes database (https://www.ncbi.nlm.nih.gov/genome/viruses/). This approach has the advantage of detecting any virus, not just those that are targeted by PCR, and the identity of the virus is determined by sequencing.

TABLE 1
Exemplary bacterial strains isolated from human fecal material that can be used alone to practice methods
as provided herein, or in making or using combinations of microbe compositions as provided herein.

| | | | | | Distance from |
|---|---|---|---|---|---|
| | | NCBI | | NCBI | Reference |
| Strain | Screening | Taxonomy | | Infraspecific | Assembly |
| ID | Medium | ID | NCBI Organism Name^{a} | Name | (mash) |
| 1 | ABB | 742722 | Collinsella sp. 4_8_47FAA | 4_8_47FAA | 0.0473307 |
| 2 | ABB | 2292944 | Bacteroides sp. AM25-34 | AM25-34 | 0.00558143 |
| 3 | ABB | 1073351 | Bacteroides stercoris CC31F | CC31F | 0.0198874 |
| 4 | ABB | 1339345 | Parabacteroides distasonis str. 3999B T(B) 6 | 3999B T(B) 6 | 0.00787841 |
| 5 | ABB | 1073351 | Bacteroides stercoris CC31F | CC31F | 0.021248 |
| 6 | ABB | 1335613 | Gordonibacter urolithinfaciens | DSM 27213T | 0.00456858 |
| 7 | ABB | 2292910 | Alistipes sp. AF14-19 | AF14-19 | 0.0168764 |
| 8 | ABB | 742722 | Collinsella sp. 4_8_47FAA | 4_8_47FAA | 0.0417882 |
| 9 | ABB | 47678 | Bacteroides caccae | OM05-21BH | 0.05067 |
| 10 | ABB | 47678 | Bacteroides caccae | OM05-21BH | 0.0121561 |
| 11 | ABB | 2292944 | Bacteroides sp. AM25-34 | AM25-34 | 0.00593905 |
| 12 | ABB | 2292944 | Bacteroides sp. AM25-34 | AM25-34 | 0.00583101 |
| 13 | ABB | 2292316 | Collinsella sp. AM34-10 | AM34-10 | 0.0529596 |
| 14 | ABB | 471875 | Ruminococcus lactaris ATCC 29176 | ATCC 29176 | 0.0131398 |
| 15 | ABB | 28116 | Bacteroides ovatus | AM40-4 | 0.0117158 |
| 16 | ABB | 997891 | Bacteroides vulgatus CL09T03C04 | CL09T03C04 | 0.0141644 |
| 17 | ABB | 742722 | Collinsella sp. 4_8_47FAA | 4_8_47FAA | 0.0447107 |
| 18 | ABB | 2292316 | Collinsella sp. AM34-10 | AM34-10 | 0.0538719 |
| 19 | ABB | 1680 | Bifidobacterium adolescentis | 2789STDY5608862 | 0.0147396 |
| 20 | ABB | 1339345 | Parabacteroides distasonis str. 3999B T(B) 6 | 3999B T(B) 6 | 0.00817721 |
| 21 | ABB | 2292236 | Odoribacter sp. AF15-53 | AF15-53 | 0.011787 |
| 22 | ABB | 46503 | Parabacteroides merdae | AM48-24BH | 0.0113184 |
| 23 | ABB | 88431 | Dorea longicatena | AF17-8AC | 0.0165507 |
| 24 | ABB | 2292944 | Bacteroides sp. AM25-34 | AM25-34 | 0.00946265 |
| 25 | ABB | 1681 | Bifidobacterium bifidum | 2789STDY5608877 | 0.160172 |
| 26 | ABB | 2292944 | Bacteroides sp. AM25-34 | AM25-34 | 0.116478 |
| 27 | ABB | 454154 | Paraprevotella clara | AF15-8 | 0.0236678 |
| 28 | ABB | 742722 | Collinsella sp. 4_8_47FAA | 4_8_47FAA | 0.0439329 |
| 29 | ABB | 997891 | Bacteroides vulgatus CL09T03C04 | CL09T03C04 | 0.0125382 |
| 30 | ABB | 821 | Bacteroides vulgatus | AM39-10 | 0.0105456 |
| 31 | ABB | 997891 | Bacteroides vulgatus CL09T03C04 | CL09T03C04 | 0.0126174 |
| 32 | ABB | 2292303 | Clostridium sp. AM30-24 | AM30-24 | 0.0326468 |
| 33 | ABB | 2292316 | Collinsella sp. AM34-10 | AM34-10 | 0.0537876 |
| 34 | ABB | 2109334 | Blautia sp. SG-772 | SG-772 | 0.0332125 |
| 35 | ABB | 454154 | Paraprevotella clara | AF15-8 | 0.0238471 |
| 36 | ABB | 2109334 | Blautia sp. SG-772 | SG-772 | 0.0255631 |
| 37 | ABB | 2292944 | Bacteroides sp. AM25-34 | AM25-34 | 0.0114769 |
| 38 | ABB | 33039 | [Ruminococcus] torques | 2789STDY5608867 | 0.0279573 |
| 39 | ABB | 1160721 | Ruminococcus bicirculans | 80/3 | 0.0243949 |
| 40 | ABB | 2109686 | Butyricicoccus sp. GAM44 | GAM44 | 0.0264344 |
| 41 | ABB | 2109334 | Blautia sp. SG-772 | SG-772 | 0.0246868 |
| 42 | ABB | 2293190 | Ruminococcus sp. AM26-12LB | AM26-12LB | 0.0196594 |
| 43 | ABB | 820 | Bacteroides uniformis | DSM 6597 | 0.0115705 |
| 44 | ABB | 411485 | Faecalibacterium prausnitzii M21/2 | M21/2 | 0.0299116 |
| 45 | ABB | 39491 | [Eubacterium] rectale | T1-815 | 0.0237145 |
| 46 | ABB | 28116 | Bacteroides ovatus | AF04-46 | 0.0211933 |
| 47 | ABB | 742722 | Collinsella sp. 4_8_47FAA | 4_8_47FAA | 0.0448295 |
| 48 | ABB | 39488 | Anaerobutyricum hallii | | 0.0309762 |
| 49 | ABB | 2292372 | Ruminococcus sp. AM42-11 | AM42-11 | 0.0259854 |
| 50 | ABB | 88431 | Dorea longicatena | 2789STDY5608851 | 0.015968 |
| 51 | ABB | 216816 | Bifidobacterium longum | DPC6320 | 0.0151441 |
| 52 | ABB | 216816 | Bifidobacterium longum | DPC6320 | 0.203899 |
| 53 | ABB | 649756 | Anaerostipes hadrus | 2789STDY5834860 | 0.0183835 |
| 54 | ABB | 216816 | Bifidobacterium longum | DPC6320 | 0.0143493 |
| 55 | ABB | 2292976 | Blautia sp. AM42-2 | AM42-2 | 0.0212548 |
| 56 | ABB | 818 | Bacteroides thetaiotaomicron | NLAE-zl-C579 | 0.0111348 |
| 57 | ABB | 2292944 | Bacteroides sp. AM25-34 | AM25-34 | 0.0058836 |
| 58 | ABB | 1504823 | bacterium LF-3 | | 0.0156336 |
| 59 | ABB | 1520805 | Blautia sp. SF-50 | SF-50 | 0.0184835 |
| 60 | ABB | 39491 | [Eubacterium] rectale | T1-815 | 0.0231774 |
| 61 | ABB | 28116 | Bacteroides ovatus | AF29-12 | 0.00552489 |
| 62 | ABB | 47678 | Bacteroides caccae | OM05-21BH | 0.0123645 |
| 63 | ABB | 47678 | Bacteroides caccae | OM05-21BH | 0.0127433 |
| 64 | ABB | 88431 | Dorea longicatena | 2789STDY5608851 | 0.0155486 |
| 65 | ABB | 1547 | Erysipelatoclostridium ramosum | OF04-4AC | 0.059652 |
| 66 | ABB | 1138888 | Enterococcus faecium EnGen0015 | E1007 | 0.0076997 |
| 67 | ABB | 997891 | Bacteroides vulgatus CL09T03C04 | CL09T03C04 | 0.0126509 |
| 68 | ABB | 1138888 | Enterococcus faecium EnGen0015 | E1007 | 0.00801624 |
| 69 | ABB | 1073351 | Bacteroides stercoris CC31F | CC31F | 0.0211864 |
| 70 | ABB | 997891 | Bacteroides vulgatus CL09T03C04 | CL09T03C04 | 0.0132351 |
| 71 | ABB | 820 | Bacteroides uniformis | DSM 6597 | 0.012262 |
| 72 | ABB | 410072 | Coprococcus comes | 2789STDY5608832 | 0.0177664 |
| 73 | YCFACB | 39485 | [Eubacterium] eligens | AF41-18 | 0.0460076 |
| 74 | YCFACB | 88431 | Dorea longicatena | 2789STDY5608851 | 0.0481542 |
| 75 | YCFACB | 2292357 | Faecalibacterium sp. OM04-11BH | OM04-11BH | 0.0598966 |
| 76 | YCFACB | 1350472 | Bifidobacterium longum subsp. longum 7-1B | 7-1B | 0.0517639 |
| 76 | YCFACB | 748224 | Faecalibacterium cf. prausnitzii KLE1255 | KLE1255 | 0.0426093 |
| 77 | YCFACB | 88431 | Dorea longicatena | 2789STDY5608851 | 0.0471561 |
| 78 | YCFACB | 88431 | Dorea longicatena | 2789STDY5608851 | 0.0471561 |
| 79 | YCFACB | 1073376 | Ruminococcus lactaris CC59_002D | CC59_002D | 0.0436095 |
| 80 | YCFACB | 1917876 | Blautia sp. Marseille-P3087 | Marseille-P3087 | 0.0581289 |
| 81 | YCFACB | 2086273 | Subdoligranulum sp. APC924/74 | APC924/74 | 0.0631331 |
| 82 | YCFACB | 2086273 | Subdoligranulum sp. APC924/74 | APC924/74 | 0.0585937 |
| 83 | YCFACB | 39491 | [Eubacterium] rectale | 2789STDY5608860 | 0.0551094 |
| 117 | ABB+ RF | 33039 | [Ruminococcus] torques | 2789STDY5608867 | 0.0201569 |
| 85 | YCFACB | 2086273 | Subdoligranulum sp. APC924/74 | APC924/74 | 0.0549626 |
| 85 | YCFACB | 2292357 | Faecalibacterium sp. OM04-11BH | OM04-11BH | 0.0625862 |
| 86 | YCFACB | 39485 | [Eubacterium] eligens | AF41-18 | 0.0480639 |
| 87 | YCFACB | 748224 | Faecalibacterium cf. prausnitzii KLEI255 | KLE1255 | 0.0647989 |
| 88 | YCFACB | 1073376 | Ruminococcus lactaris CC59_002D | CC59_002D | 0.0563141 |
| 89 | YCFACB | 39485 | [Eubacterium] eligens | AF41-18 | 0.0565927 |
| 90 | YCFACB | 515619 | [Eubacterium] rectale ATCC 33656 | ATCC 33656 | 0.0641779 |
| 91 | ABB + RF | 2292969 | Blautia sp. AM16-16B | AM16-16B | 0.207695 |
| 92 | ABB + RF | 1907658 | Bacteroides ilei | Marseille-P3208 | 0.168518 |
| 95 | ABB + RF | 214856 | Alistipes finegoldii | 2789STDY5608890 | 0.0243467 |
| 110 | ABB + RF | 2153227 | Lactobacillus sp. DS22_6 | DS22_6 | 0.00438886 |
| 93 | ABB + RF | 214856 | Alistipes finegoldii | 2789STDY5608890 | 0.0317272 |
| 94 | ABB + RF | 214856 | Alistipes finegoldii | 2789STDY5608890 | 0.0445532 |
| 96 | ABB + RF | 820 | Bacteroides uniformis | OM07-9 | 0.0172799 |
| 97 | ABB + RF | 357276 | Bacteroides dorei | An16 | 0.0141874 |
| 98 | ABB + RF | 214856 | Alistipes finegoldii | 2789STDY5608890 | 0.015699 |
| 99 | ABB + RF | 2292910 | Alistipes sp. AF14-19 | AF14-19 | 0.0155836 |
| 100 | ABB + RF | 74426 | Collinsella aerofaciens | 2789STDY5608842 | 0.0424285 |
| 101 | ABB + RF | 214856 | Alistipes finegoldii | 2789STDY5608890 | 0.0116057 |
| 102 | ABB + RF | 28118 | Odoribacter splanchnicus | AF36-2 | 0.00844432 |
| 103 | ABB + RF | 74426 | Collinsella aerofaciens | 2789STDY5608842 | 0.0432902 |
| 104 | ABB + RF | 717959 | Alistipes shahii WAL 8301 | WAL 8301 | 0.0166515 |
| 105 | ABB + RF | 2109688 | Clostridiales bacterium CCNA10 | CCNA10 | 0.114893 |
| 106 | ABB + RF | 2293194 | Ruminococcus sp. AM28-13 | AM28-13 | 0.0253257 |
| 107 | ABB + RF | 28118 | Odoribacter splanchnicus | AF36-2 | 0.00863912 |
| 108 | ABB + RF | 1871021 | Lachnoclostridium phocaeense | Marseille-P3177 | 0.0176872 |
| 109 | ABB + RF | 411471 | Subdoligranulum variabile DSM 15176 | DSM 15176 | 0.0987184 |
| 111 | ABB + RF | 28116 | Bacteroides ovatus | AF20-9LB | 0.0209153 |
| 112 | ABB + RF | 214856 | Alistipes finegoldii | 2789STDY5608890 | 0.011059 |
| 113 | ABB + RF | 2292910 | Alistipes sp. AF14-19 | AF14-19 | 0.0149744 |
| 114 | ABB + RF | 357276 | Bacteroides dorei | An16 | 0.0129382 |
| 115 | ABB + RF | 28116 | Bacteroides ovatus | AF24-28LB | 0.00894456 |
| 116 | ABB + RF | 357276 | Bacteroides dorei | An16 | 0.012209 |
| 118 | ABB + RF | 93975 | Bacteroides sp. AR29 | AR29 | 0.00583626 |
| 119 | ABB + RF | 357276 | Bacteroides dorei | An16 | 0.0121318 |
| 120 | ABB + RF | 537012 | Bacteroides cellulosilyticus DSM 14838 | DSM 14838 | 0.0196531 |
| 121 | ABB + RF | 457415 | Synergistes sp. 3_1_synl | 3_1_synl | 0.0177098 |
| 122 | ABB + RF | 33039 | [Ruminococcus] torques | AM22-16 | 0.0983271 |
| 123 | ABB + RF | 214856 | Alistipes finegoldii | 2789STDY5608890 | 0.0109684 |
| 124 | ABB + RF | 1605 | Lactobacillus animalis | P38 | 0.038387 |
| 125 | ABB + RF | 2108523 | Lawsonibacter asaccharolyticus | 3BBH22 | 0.0167368 |
| 126 | ABB + RF | 40520 | Blautia obeum | 2789STDY5834861 | 0.0656918 |
| 127 | ABB + RF | 40520 | Blautia obeum | 2789STDY5834861 | 0.0698723 |
| 128 | ABB + RF | 820 | Bacteroides uniformis | OM07-9 | 0.0156336 |
| 129 | ABB + RF | 46503 | Parabacteroides merdae | AM26-6AC | 0.0107148 |
| 130 | ABB + RF | 1871021 | Lachnoclostridium phocaeense | Marseille-P3177 | 0.0176477 |
| 131 | ABB + RF | 871324 | Bacteroides stercorirosoris | OF03-9BH | 0.0133266 |
| 132 | ABB + RF | 1339343 | Parabacteroides distasonis str. 3776 D15 iv | 3776 D15 iv | 0.0123851 |
| 133 | ABB + RF | 1339343 | Parabacteroides distasonis str. 3776 D15 iv | 3776 D15 iv | 0.012998 |
| 134 | ABB + RF | 820 | Bacteroides uniformis | OM07-9 | 0.0152174 |
| 135 | ABB + RF | 2153227 | Lactobacillus sp. DS22_6 | DS22_6 | 0.00381963 |
| 136 | ABB + RF | 216816 | Bifidobacterium longum | APC1472 | 0.0129809 |
| 137 | ABB + RF | 216816 | Bifidobacterium longum | APC1472 | 0.0133747 |
| 138 | ABB + RF | 2153227 | Lactobacillus sp. DS22_6 | DS22_6 | 0.0021922 |
| 139 | ABB + RF | 84112 | Eggerthella lenta | CC8/6 D5 4 | 0.051842 |
| 141 | ABB + RF | 46503 | Parabacteroides merdae | AF33-34 | 0.0418058 |
| 141 | ABB + RF | 40520 | Blautia obeum | 2789STDY5834957 | 0.0215309 |
| 143 | ABB + RF | 40520 | Blautia obeum | 2789STDY5834957 | 0.0421419 |
| 146 | ABB + RF | 40520 | Blautia obeum | 2789STDY5834957 | 0.0479964 |
| 147 | ABB + RF | 2292330 | Collinsella sp. TF05-9AC | TF05-9AC | 0.0755452 |
| 148 | ABB + RF | 357276 | Bacteroides dorei | OF04-10BH | 0.0311421 |
| 151 | ABB + RF | 2305245 | Clostridiaceae bacterium TF01-6 | TF01-6 | 0.0354795 |
| 152 | ABB + RF | 46503 | Parabacteroides merdae | AF33-34 | 0.00811509 |
| 153 | ABB + RF | 47678 | Bacteroides caccae | AM16-49B | 0.0324692 |
| 154 | ABB + RF | 2292271 | Lachnospiraceae bacterium AM48-27BH | AM48-27BH | 0.115114 |
| 155 | ABB + RF | 2109334 | Blautia sp. SG-772 | SG-772 | 0.0491926 |
| 157 | ABB + RF | 2109334 | Blautia sp. SG-772 | SG-772 | 0.0500057 |
| 158 | ABB + RF | 2293120 | Parabacteroides sp. AM25-14 | AM25-14 | 0.0330546 |
| 160 | ABB + RF | 476272 | Blautia hydrogenotrophica DSM 10507 | DSM 10507 | 0.0320835 |
| 161 | ABB + RF | 40520 | Blautia obeum | 2789STDY5834957 | 0.0213648 |
| 162 | ABB + RF | 33039 | [Ruminococcus] torques | 2789STDY5608833 | 0.0616729 |
| 163 | ABB + RF | 357276 | Bacteroides dorei | OF04-10BH | 0.0166462 |
| 165 | ABB + RF | 2292372 | Ruminococcus sp. AM42-11 | AM42-11 | 0.0625862 |
| 166 | ABB + RF | 2292041 | Dorea sp. AF36-15AT | AF36-15AT | 0.0479066 |
| 167 | ABB + RF | 649756 | Anaerostipes hadrus | 2789STDY5608868 | 0.0381896 |
| 169 | ABB + RF | 291644 | Bacteroides salyersiae | 2789STDY5608871 | 0.0128024 |
| 170 | ABB + RF | 33039 | [Ruminococcus] torques | 2789STDY5608833 | 0.0440096 |
| 171 | ABB + RF | 2292316 | Collinsella sp. AM34-10 | AM34-10 | 0.0316248 |
| 172 | ABB + RF | 2026190 | Bacillus mobilis | 0711P9-1 | 0.0365402 |
| 173 | ABB + RF | 47678 | Bacteroides caccae | ATCC 43185 | 0.0133309 |
| 174 | ABB + RF | 2292041 | Dorea sp. AF36-15AT | AF36-15AT | 0.0604284 |
| 175 | ABB + RF | 47678 | Bacteroides caccae | AM16-49B | 0.0423206 |
| 176 | ABB + RF | 357276 | Bacteroides dorei | OF04-10BH | 0.031968 |
| 177 | ABB + RF | 47678 | Bacteroides caccae | AM16-49B | 0.0296094 |
| 178 | ABB + RF | 39486 | Dorea formicigenerans | AF36-1BH | 0.0394151 |
| 179 | ABB + RF | 291644 | Bacteroides salyersiae | 2789STDY5608871 | 0.0250827 |
| 180 | ABB + RF | 33039 | [Ruminococcus] torques | 2789STDY5608833 | 0.0187091 |
| 181 | ABB + RF | 357276 | Bacteroides dorei | OF04-10BH | 0.0179373 |
| 182 | ABB + RF | 40520 | Blautia obeum | AM18-2AC | 0.0393356 |
| 183 | ABB + RF | 33039 | [Ruminococcus] torques | 2789STDY5608833 | 0.0186138 |
| 184 | ABB + RF | 2292992 | Catenibacterium sp. AM22-6LB | AM22-6LB | 0.063331 |
| 185 | ABB + RF | 742738 | Flavonifractor plautii 1_3_50AFAA | 1_3_50AFAA | 0.0462584 |
| 186 | ABB + RF | 476272 | Blautia hydrogenotrophica DSM 10507 | DSM 10507 | 0.0312646 |
| 187 | ABB + RF | 2292041 | Dorea sp. AF36-15AT | AF36-15AT | 0.0644247 |
| 188 | ABB + RF | 476272 | Blautia hydrogenotrophica DSM 10507 | DSM 10507 | 0.00299106 |
| 189 | ABB + RF | 1339350 | Bacteroides vulgatus str. 3775 SL(B) 10 (iv) | 3775 SL(B) 10 (iv) | 0.0288871 |
| 190 | ABB + RF | 476272 | Blautia hydrogenotrophica DSM 10507 | DSM 10507 | 0.00287005 |
| 191 | ABB + RF | 357276 | Bacteroides dorei | OF04-10BH | 0.00672075 |
| 192 | ABB + RF | 33039 | [Ruminococcus] torques | 2789STDY5608833 | 0.0189436 |
| 193 | ABB + RF | 33039 | [Ruminococcus] torques | 2789STDY5608833 | 0.0195966 |
| 194 | ABB + RF | 291644 | Bacteroides salyersiae | 2789STDY5608871 | 0.0308224 |
| 195 | ABB + RF | 2292041 | Dorea sp. AF36-15AT | AF36-15AT | 0.0641779 |
| 196 | ABB + RF | 2292271 | Lachnospiraceae bacterium AM48-27BH | AM48-27BH | 0.0957194 |
| 197 | ABB + RF | 33039 | [Ruminococcus] torques | 2789STDY5608833 | 0.0197225 |
| 198 | ABB + RF | 40520 | Blautia obeum | 2789STDY5834957 | 0.0243949 |
| 199 | ABB + RF | 997890 | Bacteroides uniformis CL03T12C37 | CL03T12C37 | 0.0228174 |
| 200 | ABB + RF | 2292330 | Collinsella sp. TF05-9AC | TF05-9AC | 0.0741211 |
| 201 | ABB + RF | 292800 | Flavonifractor plautii | 2789STDY5834932 | 0.0427185 |
| 202 | ABB + RF | 997890 | Bacteroides uniformis CL03T12C37 | CL03T12C37 | 0.0075679 |
| 203 | ABB + RF | 33039 | [Ruminococcus] torques | 2789STDY5608833 | 0.0182258 |
| 204 | ABB + RF | 40520 | Blautia obeum | OM06-11AA | 0.0550506 |
| 205 | ABB + RF | 33039 | [Ruminococcus] torques | 2789STDY5608833 | 0.0194467 |
| 206 | ABB + RF | 2292372 | Ruminococcus sp. AM42-11 | AM42-11 | 0.0486105 |
| 207 | ABB + RF | 357276 | Bacteroides dorei | OF04-10BH | 0.0219313 |
| 208 | ABB + RF | 2292330 | Collinsella sp. TF05-9AC | TF05-9AC | 0.0388799 |
| 209 | ABB + RF | 357276 | Bacteroides dorei | OF04-10BH | 0.0270028 |
| 210 | ABB + RF | 2292330 | Collinsella sp. TF05-9AC | TF05-9AC | 0.0396229 |
| 211 | ABB + RF | 649756 | Anaerostipes hadrus | 2789STDY5608868 | 0.0577367 |
| 212 | ABB + RF | 357276 | Bacteroides dorei | OF04-10BH | 0.00644046 |
| 213 | ABB + RF | 649756 | Anaerostipes hadrus | 2789STDY5608868 | 0.0187271 |
| 215 | ABB + RF | 88431 | Dorea longicatena | OM02-16 | 0.041336 |
| 216 | ABB + RF | 28116 | Bacteroides ovatus | AM32-14LB | 0.0215518 |
| 220 | ABB + RF | 2293220 | Ruminococcus sp. AM46-18 | AM46-18 | 0.0498603 |
| 221 | ABB + RF | 40520 | Blautia obeum | APC942/31-1 | 0.045432 |
| 222 | ABB + RF | 84112 | Eggerthella lenta | CC8/6 D5 4 | 0.0316362 |
| 223 | ABB + RF | 821 | Bacteroides vulgatus | AF28-7 | 0.0382047 |
| 227 | ABB + RF | 40520 | Blautia obeum | AF21-24 | 0.0354526 |
| 228 | ABB + RF | 665950 | Lachnospiraceae bacterium 3_1_46FAA | 3_1_46FAA | 0.0556449 |
| 229 | ABB + RF | 226186 | Bacteroides thetaiotaomicron VPI-5482 | VPI-5482 | 0.0228025 |
| 230 | ABB + RF | 471189 | Gordonibacter pamelaeae | 3C | 0.0276437 |
| 231 | ABB + RF | 84112 | Eggerthella lenta | CC8/6 D5 4 | 0.0280918 |
| 232 | ABB + RF | 665950 | Lachnospiraceae bacterium 3146FAA | 3146FAA | 0.0579648 |
| 233 | ABB + RF | 821 | Bacteroides vulgatus | AF28-7 | 0.0405226 |
| 234 | ABB + RF | 742738 | Flavonifractor plautii 1_3_50AFAA | 1_3_50AFAA | 0.0337178 |
| 235 | ABB + RF | 742738 | Flavonifractor plautii 1_3_50AFAA | 1_3_50AFAA | 0.0297444 |
| 236 | ABB + RF | 74426 | Collinsella aerofaciens | 2789STDY5608842 | 0.0768508 |
| 237 | ABB + RF | 74426 | Collinsella aerofaciens | 2789STDY5608823 | 0.0661271 |
| 238 | ABB + RF | 1720194 | Clostridium sp. AT4 | AT5 | 0.0475507 |
| 239 | ABB + RF | 471189 | Gordonibacter pamelaeae | 3C | 0.0393992 |
| 240 | ABB + RF | 411462 | Dorea longicatena DSM 13814 | DSM 13814 | 0.0575426 |
| 241 | RCM | 1504823 | bacterium LF-3 | | 0.0156336 |
| 242 | RCM | 33038 | [Ruminococcus] gnavus | RJX1120 | 0.022603 |
| 243 | RCM | 33039 | [Ruminococcus] torques | 2789STDY5608867 | 0.0235981 |
| 244 | RCM | 33039 | [Ruminococcus] torques | 2789STDY5608867 | 0.0273626 |
| 245 | RCM | 33039 | [Ruminococcus] torques | 2789STDY5608833 | 0.0309541 |
| 246 | RCM | 33039 | [Ruminococcus] torques | 2789STDY5608833 | 0.0267663 |
| 247 | RCM | 33039 | [Ruminococcus] torques | 2789STDY5608833 | 0.0285595 |
| 248 | RCM | 39488 | Anaerobutyricum hallii | | 0.0430304 |
| 249 | RCM | 39488 | Anaerobutyricum hallii | AF45-14BH | 0.0321067 |
| 250 | RCM | 1532 | Blautia coccoides | NCTC11035 | 0.022559 |
| 251 | RCM | 476272 | Blautia hydrogenotrophica DSM 10507 | DSM 10507 | 0.108521 |
| 252 | RCM | 476272 | Blautia hydrogenotrophica DSM 10507 | DSM 10507 | 0.0213993 |
| 253 | RCM | 40520 | Blautia obeum | 2789STDY5608837 | 0.0222102 |
| 254 | RCM | 40520 | Blautia obeum | AM37-4AC | 0.0291893 |
| 255 | RCM | 40520 | Blautia obeum | OF03-14 | 0.0315342 |
| 256 | RCM | 410072 | Coprococcus comes | 2789STDY5834962 | 0.0318186 |
| 257 | RCM | 410072 | Coprococcus comes | 2789STDY5608832 | 0.0375188 |
| 258 | RCM | 410072 | Coprococcus comes | 2789STDY5834962 | 0.0339433 |
| 259 | RCM | 410072 | Coprococcus comes | 2789STDY5608832 | 0.0324692 |
| 260 | RCM | 39486 | Dorea formicigenerans | AF19-13 | 0.0283927 |
| 261 | RCM | 39486 | Dorea formicigenerans | AF19-13 | 0.0245322 |
| 262 | RCM | 39486 | Dorea formicigenerans | AF19-13 | 0.0306047 |
| 263 | RCM | 39486 | Dorea formicigenerans | TF12-1 | 0.0844968 |
| 264 | RCM | 39486 | Dorea formicigenerans | TF12-1 | 0.013909 |
| 265 | RCM | 39486 | Dorea formicigenerans | TF12-1 | 0.0367526 |
| 266 | RCM | 88431 | Dorea longicatena | 2789STDY5608851 | 0.0210911 |
| 267 | RCM | 88431 | Dorea longicatena | 2789STDY5608851 | 0.026948 |
| 268 | RCM | 88431 | Dorea longicatena | OM02-16 | 0.0378742 |
| 269 | RCM | 88431 | Dorea longicatena | 2789STDY5834914 | 0.0338178 |
| 270 | RCM | 88431 | Dorea longicatena | OM02-16 | 0.037681 |
| 271 | RCM | 88431 | Dorea longicatena | OM02-16 | 0.0381896 |
| 272 | RCM | 88431 | Dorea longicatena | 2789STDY5608851 | 0.0314102 |
| 273 | RCM | 411462 | Dorea longicatena DSM 13814 | DSM 13814 | 0.0304105 |
| 274 | RCM | 2292041 | Dorea sp. AF36-15AT | AF36-15AT | 0.035887 |
| 275 | RCM | 2292041 | Dorea sp. AF36-15AT | AF36-15AT | 0.0313653 |
| 276 | RCM | 28052 | Lachnospira pectinoschiza | 2789STDY5834886 | 0.0345299 |
| 277 | RCM | 1160721 | Ruminococcus bicirculans | 80/3 | 0.0394469 |
| 278 | RCM | 2293190 | Ruminococcus sp. AM26-12LB | AM26-12LB | 0.0238706 |
| 279 | RCM | 2292372 | Ruminococcus sp. AM42-11 | AM42-11 | 0.0346335 |
| 280 | RCM | 2292372 | Ruminococcus sp. AM42-11 | AM42-11 | 0.0305938 |
| 281 | RCM | 2292372 | Ruminococcus sp. AM42-11 | AM42-11 | 0.0353051 |
| 282 | RCM | 2292372 | Ruminococcus sp. AM42-11 | AM42-11 | 0.0292401 |
| 283 | ActVeg | 457422 | Erysipelotrichaceae bacterium 2_2_44A | 2_2_44A | 0.0120469 |
| 284 | ActVeg | 1597 | Lactobacillus paracasei | 1316.rep1_LPAR | 0.0122212 |
| 285 | ActVeg | 573236 | Bifidobacterium animalis subsp. lactis V9 | V9 | 0.0122825 |
| 286 | ActVeg | 1522 | [Clostridium] innocuum | AF18-35LB | 0.0163194 |
| 287 | ActVeg | 457422 | Erysipelotrichaceae bacterium 2_2_44A | 2_2_44A | 0.0170934 |
| 288 | ActVeg | 84112 | Eggerthella lenta | CC8/6 D5 4 | 0.0177777 |
| 289 | ActVeg | 649756 | Anaerostipes hadrus | 2789STDY5608868 | 0.0237534 |
| 290 | ActVeg | 39486 | Dorea formicigenerans | TF12-1 | 0.0244433 |
| 291 | ActVeg | 410072 | Coprococcus comes | 2789STDY5834962 | 0.0245972 |
| 292 | ActVeg | 410072 | Coprococcus comes | 2789STDY5834962 | 0.0252752 |
| 293 | ActVeg | 33035 | Blautia producta | DSM 3507 | 0.0263101 |
| 294 | ActVeg | 2293194 | Ruminococcus sp. AM28-13 | AM28-13 | 0.0269024 |
| 295 | ActVeg | 410072 | Coprococcus comes | 2789STDY5834962 | 0.0277667 |
| 296 | ActVeg | 457412 | Ruminococcus sp. 5139BFAA | 5139BFAA | 0.0293319 |
| 297 | ActVeg | 100884 | Coprobacillus cateniformis | OM02-34 | 0.0320488 |
| 298 | ActVeg | 39486 | Dorea formicigenerans | AF36-1BH | 0.0344011 |
| 299 | ActVeg | 2293184 | Ruminococcus sp. AM16-34 | AM16-34 | 0.0374015 |
| 300 | ActVeg | 1870991 | Massilioclostridium coli | Marseille-P2976 | 0.0377106 |
| 301 | ActVeg | 665951 | Lachnospiraceae bacterium 8_1_57FAA | 8_1_57FAA | 0.0377551 |
| 302 | ActVeg | 665950 | Lachnospiraceae bacterium 3_1_46FAA | 3_1_46FAA | 0.0381292 |
| 303 | ActVeg | 2302976 | Erysipelotrichaceae bacterium AF19-24AC | AF19-24AC | 0.0434587 |
| 304 | ActVeg | 649724 | Clostridium sp. ATCC BAA-442 | ATCC BAA-442 | 0.0446515 |
| 305 | ActVeg | 74426 | Collinsella aerofaciens | 2789STDY5608823 | 0.0474845 |
| 306 | ActVeg | 74426 | Collinsella aerofaciens | 2789STDY5608842 | 0.0479739 |
| 307 | ActVeg | 665950 | Lachnospiraceae bacterium 3_1_46FAA | 3_1_46FAA | 0.0516344 |
| 308 | ActVeg | 649756 | Anaerostipes hadrus | 2789STDY5608868 | 0.0525015 |
| 309 | ActVeg | 1965564 | Massilimicrobiota sp. An142 | An142 | 0.0530685 |
| 310 | ActVeg | 2292330 | Collinsella sp. TF05-9AC | TF05-9AC | 0.0557352 |
| 311 | ActVeg | 1737424 | Blautia massiliensis | GD9 | 0.0581948 |
| 312 | ActVeg | 2292330 | Collinsella sp. TF05-9AC | TF05-9AC | 0.0649247 |
| 313 | ActVeg | 2292330 | Collinsella sp. TF05-9AC | TF05-9AC | 0.0649247 |
| 314 | ActVeg | 2292227 | Collinsella sp. AF28-5AC | AF28-5AC | 0.0929879 |
| 315 | ActVeg | 552398 | Ruminococcaceae bacterium D16 | D16 | 0.0957194 |
| 316 | ActVeg | 208479 | [Clostridium] bolteae | AM35-14 | 0.0989821 |
| 317 | ActVeg | 33039 | [Ruminococcus] torques | AM22-16 | 0.109435 |
| 318 | ActVeg | 2086584 | Mordavella sp. Marseille-P3756 | Marseille-P3756 | 0.112966 |
| 319 | ActVeg | 457412 | Ruminococcus sp. 5_1_39BFAA | 5_1_39BFAA | 0.114022 |
| 320 | ActVeg | 1121115 | Blautia wexlerae DSM 19850 | DSM 19850 | 0.129179 |
| 321 | ActVeg | 2293156 | Ruminococcus sp. AF18-29 | AF18-29 | 0.130966 |
| 322 | ActVeg | 2292372 | Ruminococcus sp. AM42-11 | AM42-11 | 0.134052 |
| 323 | ActVeg | 552398 | Ruminococcaceae bacterium D16 | D16 | 0.13446 |
| 324 | ActVeg | 1965654 | Lachnoclostridium sp. An76 | An76 | 0.13446 |
| 325 | ActVeg | 1965654 | Lachnoclostridium sp. An76 | An76 | 0.138386 |
| 326 | ActVeg | 33039 | [Ruminococcus] torques | AM22-16 | 0.139328 |
| 327 | ActVeg | 552398 | Ruminococcaceae bacterium D16 | D16 | 0.139328 |
| 328 | ActVeg | 2292376 | Ruminococcus sp. OM08-7 | OM08-7 | 0.142336 |
| 329 | ActVeg | 116085 | Coprococcus catus | AF45-17 | 0.148079 |
| 330 | ActVeg | 2292970 | Blautia sp. AM22-22LB | AM22-22LB | 0.150025 |
| 331 | ActVeg | 2292970 | Blautia sp. AM22-22LB | AM22-22LB | 0.163053 |
| 332 | ActVeg | 2293138 | Roseburia sp. AM59-24XD | AM59-24XD | 0.164074 |
| 333 | ActVeg | 1235835 | Anaerotruncus sp. G3(2012) | G3 | 0.166219 |
| 334 | ActVeg | 29348 | [Clostridium] spiroforme | OM02-6 | 0.172308 |
| 335 | ActVeg | 2292376 | Ruminococcus sp. OM08-7 | OM08-7 | 0.176605 |
| 336 | ActVeg | 2293138 | Roseburia sp. AM59-24XD | AM59-24XD | 0.183409 |

^{a}Listed are the closest genome/species matches for each strain, determined by the analysis described in the text.

Antibiotic Resistance Characterization of Isolated Strains from Fecal Matter

The complete genome sequence of each organism is screened to ensure it contains no genes or pathogenicity island gene clusters encoding known virulence factors, toxins, or antibiotic resistance functions, using publicly available databases such as DBETH55 (for example, see Chakraborty A, et al. (2012) Nucleic Acids Res. 40:615-620) and VFDB56 (Chen L, et al. (2005) Nucleic Acids Res. 33:325-328). Each organism is tested by standard antibiotic sensitivity profile techniques such as broth microdilution susceptibility panels or plate-based methods such as disk diffusion method and antimicrobial gradient method (James H. Jorgensen and Mary Jane Ferraro 2009 Clinical Infectious Diseases 49:1749-1755). Such tests determine the minimal inhibitory concentration (MIC) of an antibiotic on microbial growth. Antibiotics tested include but are not limited to amoxicillin, amoxicillin/clavulanic acid, carbapenem, methicillin, ampicillin, gentamicin, metronidazole, and neomycin. MIC determinations of novel microbes are compared to published values for both sensitive and resistant related strains to make an assessment on sensitivity (CLSI Guideline M45: Methods for Antimicrobial Dilution and Disk Susceptibility Testing of Infrequently Isolated or Fastidious Bacteria. Wayne, Pa.; 2015) to type strains of related microbes to determine possible relative increases in antibiotic resistance.

### Example 4: Isolation and Characterization of Pure Microbial Strains from Endospores Purified from Fecal Matter

In alternative embodiments, microbes used in compositions as provided herein, or used to practice methods as provided herein, are derived from, or are cultured as, pure microbial strains derived from endospores purified or derived from fecal matter.

Individual spore-forming bacterial strains can be preferentially isolated and cultured from endospores purified from fecal matter using a protocol adapted from Kearney et al 2018 ISME J. 12:2403-2416. Purified endospores are spread on solid anaerobic medium plates and allowed to germinate and form colonies that can be further characterized. Vegetative cells in the fecal matter are rendered non-viable during the endospore purification process, and thus any resulting colonies are restricted to spore-forming bacteria. Endospores are purified from fecal matter as follows:

Fecal samples are collected and processed in an anaerobic chamber within 30 minutes of defecation. Samples (5 g) are suspended in 20 mL of 1% sodium hexametaphosphate solution (a flocculant) in order to bring biomass into suspension. The suspension is bump vortexed with glass beads to homogenize and centrifuged at 50×g for 5 min at room temperature to sediment particulate matter and beads. Quadruplicate 1 mL aliquots of the supernatant liquid is transferred into cryovials and stored at −80° C. until processing.

The frozen supernatant liquid samples are thawed at 4° C., centrifuged at 4° C. and 10,000×g for 5 minutes, washed and then resuspended in 1 mL Tris-EDTA pH 7.6. The samples are heated at 65° C. for 30 minutes with shaking at 100 rpm and then cooled on ice for 5 minutes. Lysozyme (10 mg/mL) is added to a final concentration of 2 mg/mL and the samples are incubated at 37° C. for 30 minutes with shaking at 100 rpm. At 30 minutes, 50 μL Proteinase K (>600 mAU/ml) (Qiagen) is added and the samples incubated for an additional 30 minutes at 37° C. 200 μL 6% SDS, 0.3 N NaOH solution is added to each sample and incubated for 1 hour at room temperature with shaking at 100 rpm. Samples are then centrifuged at 10,000 rpm for 30 minutes. At this step, a pellet containing resistant endospores is visible, and the pellet is washed three times at 10,000×g with 1 mL chilled sterile ddH2O. The pellet containing endospores is stored at −20° C. until required.

To germinate and resuscitate spore-forming bacterial colonies from the purified endospores, the endospore pellet is brought into the anaerobic chamber, thawed and then suspended in 1.0 ml reduced ABB. Successive 10-fold serial dilutions of the suspended spores are then performed in ABB to establish 1/10, 1/100, 1/1000, 1/10000, 1/100000, 1/1000000 dilutions of the endospore preparation. From each 10-fold serial dilution, four 0.1 ml volumes are removed and then added to and spread over Reinforced Clostridial Medium Agar (Oxoid), with 0.1% intestinal bile salts (taurocholate, cholate, glycocholate) to stimulate endospore germination. The platings are incubated at 37° C. for 2, 3, 4, 5, 6, 7, 8, 9, 10, 11, 12 days to allow for the endospores to germinate and grow as single colonies. These colonies are then manually picked, individually cultivated, and then subjected to identification by whole genome sequencing analysis as described in Example 3.

### Example 5: Stability Testing

In alternative embodiments, microbes used in compositions as provided herein, or used to practice methods as provided herein, comprise or can be derived from any one of family or genus (or class): Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663).

In alternative embodiments, any microbe used in a composition as provided herein, or used to practice methods as provided herein, for example, including a microbe as listed above, can be stored in a sealed container, for example, at 25° C. or 4° C. and the container can be placed in an atmosphere having 30%, 40%, 50%, 60%, 70%, 75%, 80%, 90% or 95% relative humidity, or between about 20% and 99% relative humidity. In alternative embodiments, after 1 month, 2 months, 3 months, 6 months, 1 year, 1.5 years, 2 years, 2.5 years or 3 years, at least 50%, 60%, 70%, 80% or 90% of the bacterial strain shall remain as measured in colony forming units determined by standard protocols.

### Example 6—in Silico Modeling to Discover Microbe-Microbe Interactions

Microbe-microbe interactions are determined to exploit and manipulate metabolic reactions present in the gut microbiome using compositions and methods as provided herein for, for example, increase the ability of the immune system to combat viral infections, stimulate activity of specific classes of immune cells, provide essential nutrients that may be depleted or blocked by the virus, produce compounds with antiviral activity, or other direct or indirect effect on cells of the innate or adaptive immune system.

Genome scale metabolic modeling is used as a tool to explore the diversity of metabolic reactions present in the gut microbiome, interpret the omics data described here in the framework of cellular metabolism, and evaluate inter-species interactions. A set of 773 different organism-specific metabolic models have been created (Magnusdottir et al. Nature Biotechnology 2017, 35(1):85-89) and are used in this work. Models are used individually to predict the metabolic capabilities of each organism and combined to enable multispecies simulations that predict how these organisms interact when supplied with a nutrient mix mimicking the typical Western human diet or variations thereof. Simulations are performed using the COBRA™ package v2.0™ (Schellenberger et al., Nature Protocols 2011, 6:1290-1307) or updated versions thereof. Commensal relationships among the organisms result when one or more species consume a compound that another species produces and can be detected by an increased maximum predicted growth rate of each species when growing together than when each is grown separately. In the cases where commensalism is not predicted in the live biotherapeutics provided, simulations are used to identify a suitable microbial partner that can be included in the live biotherapeutic product, thus improving the ability of the active microbes to grow in the gut ecosystem. Similarly, simulations are used to identify prebiotic compounds to be supplemented that can be utilized by the active species as a carbon or energy source.

Metabolic models are downloaded from the Thiele lab website (https://wwwen.uni.lu/lcsb/research/mol_systems_physiology/in_silico_models) for the following organisms: Coprococcus comes, Dorea formicigenerans, Anaerostipes hadrus, Dorea longicatena, Coprococcus eutactus, Ruminococcus lactaris, Coprococcus catus, Fusicatenibacter saccharivorans, Lachnoclostridium sp. SNUG30099, Clostridium sporogenes, Eubacterium ventriosum, Blautia obeum, Erysipelotrichaceae bacterium GAM147, Akkermansia muciniphilia, Faecalibacterium prauznitzii, Ruminococcus torques, Ruminococcus gnavus, Eubacterium hallii, Blautia obeum, and Clostridium scindens. The models are then used for simulations in the COBRA v2.0™ package (Schellenberger et al., Nature Protocols 2011, 6:1290-1307). Cell metabolism is simulated by defining nutrient uptake rates (mmol/gDCW-hr) and optimizing for growth of each organism (hr⁻¹). Oxygen uptake rate is set to zero, to simulate anaerobic conditions. Values for each nutrient uptake rate are obtained from (Magnusdottir et al. Nature Biotechnology 2017, 35(1):85-89, Supplemental Table 12), as estimated for a typical Western diet. To simulate the gut ecosystem comprising of multiple bacterial species, each organism model is treated as a separate compartment, with the extracellular space in the gut considered an additional compartment. Nutrients can enter and exit the extracellular space freely, to simulate food uptake and waste excretion. Nutrients can enter and exit each microbial species based on the specific transporters present in the respective model. The objective function to be maximized is defined to be the total biomass of all species; i.e., the sum of all individual growth rates. The minimum growth rate of each species is set at 0.001 hr⁻¹.

The consortia of gut microbe metabolic models are used as a framework for interpreting genomic, transcriptomic, and metabolomic data obtained from the mouse and human studies. Enriched genes or pathways at the genomic or transcriptomic level are mapped to the source organism model to determine the metabolic functions these represent and how they connect with the rest of metabolism in that organism, as well as in the gut ecosystem. Enrichments also in metabolic intermediates or end products of these pathways provide further evidence for these pathways' contribution to checkpoint inhibitor function.

### Example 7: In Silico Simulation of Relevant Microbial Species

Models were downloaded for the following organisms: Akkermansia muciniphilia, Faecalibacterium prausnitzii, Ruminococcus torques, Ruminococcus gnavus, Ruminococcus lactaris, Eubacterium hallii, Blautia obeum, Anaerostipes hadrus, Dorea formicigenerans, Coprococcus comes, Coprocuccus catus, Erysipelotrichaceae sp., and Clostridium scindens. The models are then used for simulations in the COBRA package v2.0 (Schellenberger et al., Nature Protocols 2011, 6:1290-1307). Cell metabolism was simulated by defining nutrient uptake rates (mmol/gDCW-hr) and optimizing for growth rate of each organism (hr¹). Oxygen uptake rate was set to zero, to simulate anaerobic conditions.

First, simulations were performed to determine the minimal growth substrate requirements of each organism. Starting with all substrate uptake fluxes open, allowing utilization of any nutrient, simulations were performed as nutrient uptake fluxes are systematically removed. This was continued for each organism until a minimal set of carbon sources remained, the removal of any of which would result in zero predicted growth. Normally, this resulted in a single sugar compound (often glucose) and one or more other nutrients such as amino acids, nucleotides, vitamins, or lipids. These other compounds are considered auxotrophic requirements of the organism. Next, the substrate utilization range of the organism was determined. The uptake flux of the primary growth substrate (generally, a sugar) was set to zero, and growth was evaluated with different carbon sources one at a time. The predicted ability to grow using each carbon source was documented. The ability to co-utilize organic acid carbon sources was also evaluated. These compounds generally cannot be used as a sole growth substrate during anaerobic growth but can be taken up in conjunction with a sugar. Simulations were run with the uptake rate of each compound constrained to a non-zero value, while maintaining the uptake of the primary sugar source. If an increase was observed in the predicted growth rate over the use of the sugar alone, then co-utilization is considered to be feasible.

The capability of each strain to produce various fermentation products was evaluated using the models. Some products were predicted to naturally form during the carbon source simulations above, as fermentation products are needed to balance redox in anaerobic conditions. These products were noted. For other compounds, the model was constrained to make each one by setting the output flux to a non-zero value. If the simulation gave a feasible solution, then the organism was considered capable of making this product.

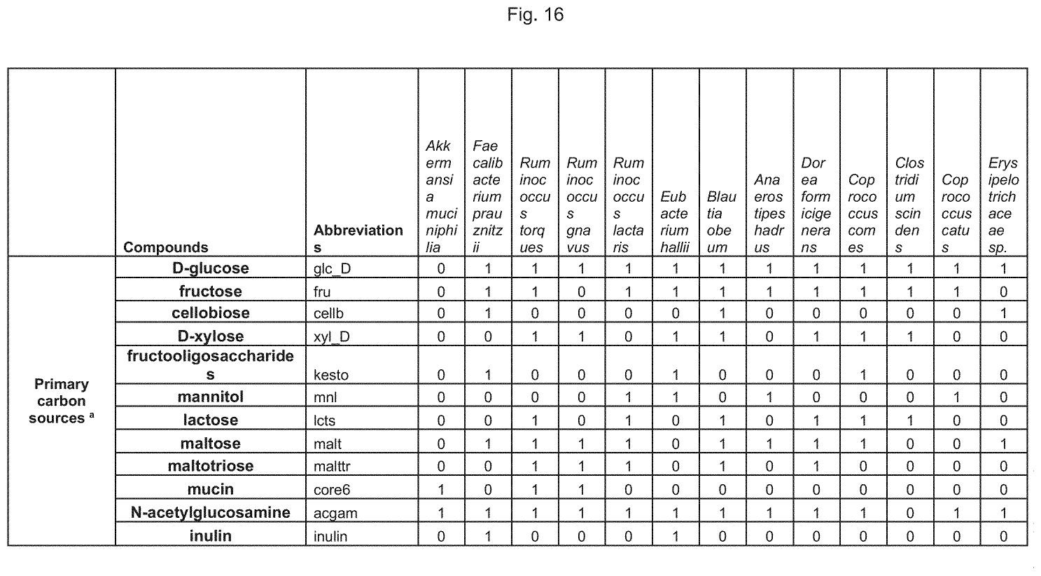

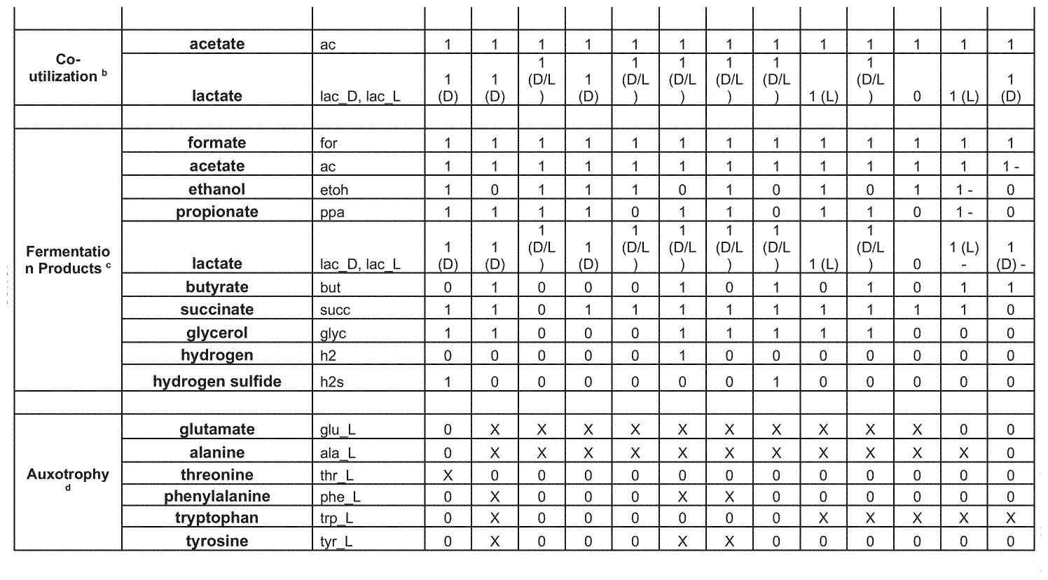

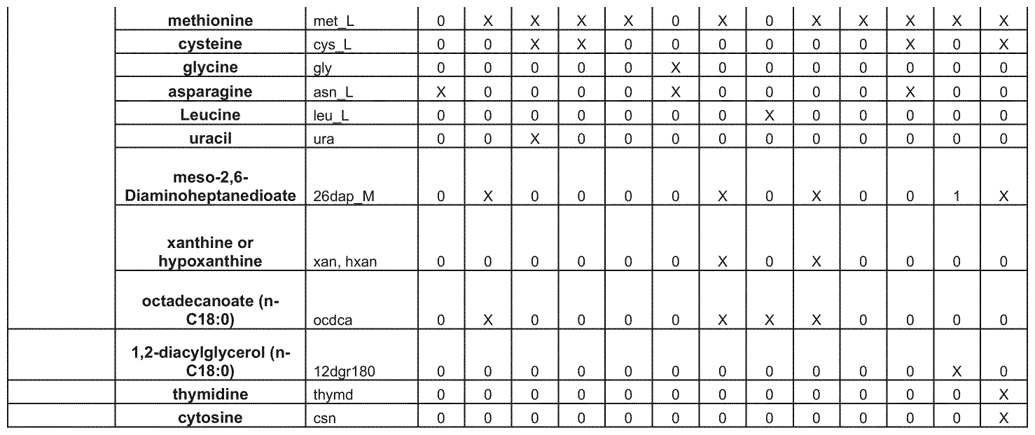

Table 2 (illustrated as FIG. 16). Simulation of selected organisms with constraint-based modeling.
^{a} 1 indicates predicted growth on substrate; 0 indicates predicted no growth
^{b} 1 indicates compound is predicted to be used as a supplemental carbon source; 0 indicates it cannot be consumed
^{c} 1 indicates that model predicts production of fermentation product is feasible; 0 indicates it is not feasible
^{d} Compounds that must be supplied in the growth media are indicated by X

### Example 8: Laboratory-Scale Fermentation of Isolated Anaerobic Microorganisms

In alternative embodiments, microbes used in compositions as provided herein, or used to practice methods as provided herein, comprise use of isolated anaerobic microorganisms, for example, anaerobic bacteria isolated from a fecal sample, for example, from a donor.

A laboratory-scale fermentation is performed using a Sartorius BIOSTAT A™ bioreactor with 2-liter (L) vessel, using the growth media described in Example 1. While still in the anaerobic chamber, 1 L media is transferred to a sterile feed bottle, which has two ports with tubing leading blocked by pinch clamps and covered in foil to maintain sterility.

The fermentation vessel is sterilized by autoclaving, then flushed with a continuous purge of sterile nitrogen gas with oxygen catalytically removed. Two inlet ports are fitted with tubing leading to a connector blocked with a pinch clamp, and the sampling port fitted with tubing leading to a syringe. The vessel is also fitted with a dissolved oxygen probe, a pH probe, and a thermowell containing a temperature probe. Once anaerobic conditions are ensured, the media is removed from the anaerobic chamber and connected to one of the inlet ports. The other feed bottle port is connected to sterile nitrogen purge. The pinch clamp is removed, and media transferred into the fermentation vessel by peristaltic pump or just by the nitrogen pressure. Once the transfer is complete, both lines are sealed again by the pinch clamps, the feed bottle removed, and returned to the anaerobic chamber.

A 50 mL seed culture of one or more bacteria from the following genera (any one of which are used to practice compositions or methods as provided herein), Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), are grown to mid-exponential phase in a sealed culture bottle using the same media composition as above, and are transferred into the feed bottle in the anaerobic chamber. Repeating the above transfer procedure, this time with the culture, the fermenter is inoculated.

5 M ammonium hydroxide is prepared in another feed bottle. One port is connected to sterile nitrogen, and the bottle is purged for 5 minutes to remove all oxygen. The outlet tubing is then blocked by a pinch clamp and attached to the other inlet port in the fermentation vessel. This tubing is then threaded into a peristaltic pump head, and the pinch clamp removed. Using the software built into the Biostat A™ unit, this pump is controlled to maintain pH at 7.0.

During growth of the culture, temperature is maintained at 37° C. using a temperature controller and heating blanket on the vessel. Nitrogen purge is set at 0.5 L/min to maintain anaerobic conditions and positive pressure in the vessel, and agitation is set at 500 rpm to keep the culture well mixed. Periodic samples are taken using the syringe attached to the sample port. For each sample, optical density is measured at 600 nm wavelength using a spectrophotometer.

### Example 9: Patient Data Collection from Clinical Trials and Machine Learning and Data Analysis on the Same

The results described here were obtained from a study involving cancer patients undergoing immunotherapy treatment and healthy controls. Microbes, gene functions, and metabolites elucidated as being absent in patients not responding well to treatment are relevant for the treatment of viral infections because in both cases a healthy immune response is required to combat the disease. Therefore, it is reasonable to expect that microbes beneficial for immuno-oncology treatment will also be beneficial or even essential for treating or ameliorating a viral infection, or for rapid viral clearance.

Eligible patients were selected based on current health condition, cancer status (current or in remission), and treatment program. Prior patient medical history was also collected and analyzed when available. This includes but is not limited to prior cancer history, diabetes, autoimmune disease, neurodegenerative disease, heart disease, metabolic syndrome, digestive disease, psychological disorders, HIV, and allergies. In addition, lifestyle and dietary habits were collected, including diet regimen, exercise routine, alcohol, nicotine, and caffeine intake, medical as well as recreational drug use, recent courses of antibiotics, vitamins, and probiotics. In some cases, information and data collected from wearable devices that monitor but is not limited to heart rate, calories burned, steps walked, blood pressure, biochemical release, time spent exercising and seizures. This data was assembled and used as input to the machine learning algorithms with the goal of determining correlations between patient history, wearable devices and treatment efficacy. In addition, relationships between this data and the results of sample analysis described below were elucidated.

In another embodiment, eligible patients testing positive for infection with COVID-19 (SARS-CoV2) or other coronavirus, or influenza virus, as well as age-matched healthy controls. Information is also collected on the severity of disease, symptoms, time of recovery, and response to any treatment, if applicable. Prior patient medical history is also collected and analyzed, including but not limited to cancer, diabetes, autoimmune disease, neurodegenerative disease, heart disease, metabolic syndrome, digestive disease, psychological disorders, coronaviruses, influenza virus, HIV, and allergies. In addition, lifestyle and dietary habits are collected, including diet regimen, exercise routine, alcohol, nicotine, and caffeine intake, medical as well as recreational drug use, recent courses of antibiotics, vitamins, and probiotics. This data is assembled and used as input to the machine learning algorithms with the goal of determining correlations between patient history, course of illness, and results of stool and blood sample analysis

For current cancer patients, tumor size and cancer progression are tracked over time and are classified based on radiographic assessment using the Response Criteria in Solid Tumors version 1.1 (Schwartz et al. Eur. J. Cancer 2016, 62:132-137) criteria. This is based on longitudinal measurements of lesions in cancer tissue, given a strict set of guidelines for lesion selection and measurement techniques. Responders to checkpoint inhibitor treatment are defined as patients that were cured or had stable disease lasting at least 6 months, while non-responders are defined as those whose cancer progressed or was stable for less than 6 months. Classification of responders and non-responders implies robust and insufficient immune response, respectively, and thus serves as a proxy for COVID-19, influenza, or other viral disease patients that will effectively clear the virus or have severe symptoms, respectively.

Each patient provided stool samples using the procedures as outlined in Example 2 and buccal swabs of the oral biome. In some cases, Urine, Blood and plasma samples were also taken by healthcare personnel within 1-2 days of the stool samples. Stool, urine and buccal samples were kept on ice or at 4° C. until processed. Whole blood was collected into an EDTA tube. Plasma was isolated from the blood by centrifugation at 1000×g for 10 minutes, followed by centrifugation at 2000×g for 10 minutes. At least three timepoints were taken for each patient, roughly every 6 to 8 weeks.

### Flow Cytometry Analysis of Peripheral Blood

Flow cytometry analysis of peripheral blood can provide a non-invasive immune profile of the patients on study (Showe et al. Cancer Res. 2009 Dec. 15; 69(24): 9202-9210). The peripheral blood immuno-profile evaluation was performed on blood samples collected from patients on study. Phenotypic markers of lymphocyte subpopulations and regulatory T cells (Tregs) was evaluated using flow cytometry with populations gated to include CD3, CD4, CD8, CD11b, CD14, CD15, CD25, CD45, CD56, HLA-DR and FoxP3-expressing cells using antibodies to each cell type (BD Biosciences). Peripheral blood cells were stained with Live/Dead violet dye (Invitrogen, Carlsbad, Calif.) to gate on live cells. Data was acquired on an LSR II™ flow cytometer (BD Biosciences) and analyzed with FLOWJO™ software (TreeStar, Ashland, Oreg.).

### Peripheral Blood Mononuclear Cell (PBMC) Preparation and CyTOF® Analysis

Peripheral blood mononuclear cells (PBMC's) are isolated from subject blood using a standard kit and stored in liquid nitrogen at 1×10\hat{ }6 cells/mL until use. Prior to storage, PBMC's may be processed using flow sorting or an antibody spin separation kit to select for a certain purified lymphocyte subpopulation, such as T cells. To characterize the immune profile of the PBMCs, single cell proteomics analysis (CyTOF®) is applied. This work is conducted by the Bioanalytical and Single-Cell Facility at the University of Texas, San Antonio, and entails a comprehensive panel of 29 different immune markers, allowing for deep interrogation of cellular phenotype and function (https://www.fluidigm.com/products/helios). To complement these results, RNA sequencing is applied to the entire population of the PBMCs, sorted populations, and also to single cells. Single cell RNAseq is applied using the method developed by 10×Genomics (https://www.10xgenomics.com/solutions/single-cell/). Finally, cytokine levels are determined using the Human Cytokine 30-Plex Luminex assay (https://www.thermofisher.com/order/catalog/product/LHC6003M).

Reassignment of Microbial Genomes into Operational Species Units Because of the limitations of the NCBI taxonomy tree, and the necessity of including proprietary microbial genome assemblies into the reference alignment sequence database, it is necessary to generate a new taxonomy of microbes. Previous work (for example, see Jain et al. (2018) Nature CommunicaGtabletions 9(1):5114) shows that species are a biologically relevant construction, with the average genomic distance (1-average nucleotide identity) between strains of a species being less than 0.04. Using this as an inspiration, all microbial assemblies from the NCBI RefSeq (Pruitt et al. (2006) Nucleic Acids Research 35(suppl_1):D61-D65) were assigned into operational species units (OSUs) based on a clustering in which microbial assemblies within a genomic distance of 0.04 are assigned to the same OSU.

All microbial assemblies belonging to bacteria and archaea were acquired from the NCBI RefSeq database. All pairwise distances were calculated between assemblies using mash (Ondov et al. (2016) Genome Biology 17(1):132). Clustering is performed using DBSCAN (Ester et al. (1996) KDD-96 96:226-231) with an epsilon parameter of 0.04. Identified clusters were denoted as operational species units (OSUs). Proprietary microbial assemblies were seamlessly included in this procedure as well.

For each OSU, an integer cluster label was created, and a new taxonomic ID created that is unique from any existing NCBI taxonomic identification numbers. The least common ancestor of each OSU was calculated using the original NCBI taxonomy IDs of its member assemblies, and each OSU taxonomic ID was inserted into the NCBI tree under its least common ancestor. Each OSU is also named using its most common species and label number (for example Bifidobacterium adolescentis C0001).

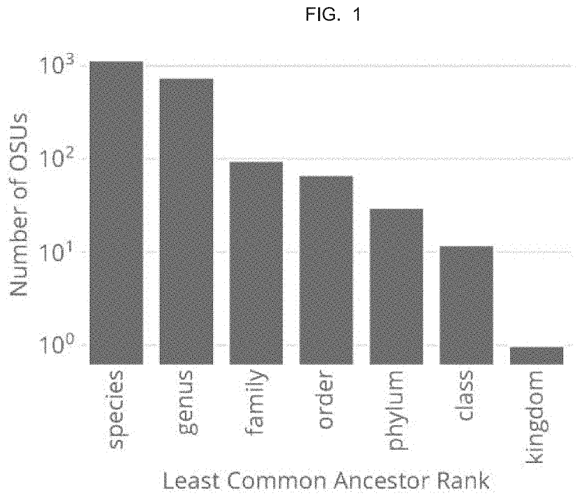

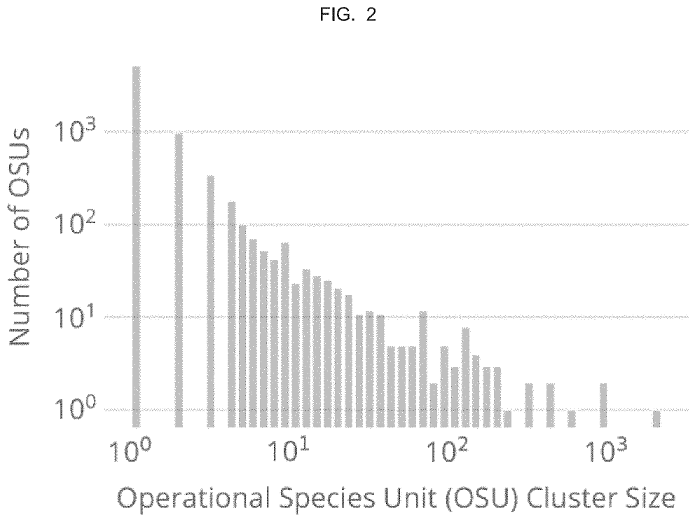

In FIG. 1, the ranks of the least common ancestor of each OSU that contains more than one assembly are displayed. Most OSUs are consistent with pre-existing NCBI taxonomy, with a least common ancestor at the species or genus level. However, for 207 out of 2,112 non-singleton OSUs, the least common ancestor is at the family level or higher. The chart in FIG. 2 demonstrates that the frequency of OSUs decreases as the cluster size increases in a log-log fashion.

The new names, reference sequences, and taxonomy were used to generate a new reference database for the alignment program centrifuge (Kim et al. (2016) Genome Research 26:1721-1729). The centrifuge program classifies sequencing reads from a metagenomic fecal sample to reference sequences and uses an expectation-maximization method to estimate relative abundance of the taxa present in the sample. The estimated relative abundances for each OSU are carried into downstream analyses, such as machine learning or differential abundance analysis.

In addition to the method for re-assigning taxonomy described, pre-built databases that use the Genome Taxonomy Database (GTDB) were directly used for centrifuge classification (Parks et al. (2019) bioRxiv 771964, Meric et al. (2019) bioRxiv 712166).

### Whole Genome Sequencing of Patient Fecal Samples

Whole genome sequencing was performed as previously described in Example 3 on a total of 387 fecal samples. Of the 387 samples, 266 samples were from cancer patients, 88 were from control subjects, and 31 were from subjects in remission. The results were classified, and abundance was estimated for each sample using centrifuge, using either a reference database built in-house consisting of operational species units (OSUs) or a publicly available one (Parks et al. (2019) bioRxiv 771964, Meric et al. (2019) bioRxiv 712166).

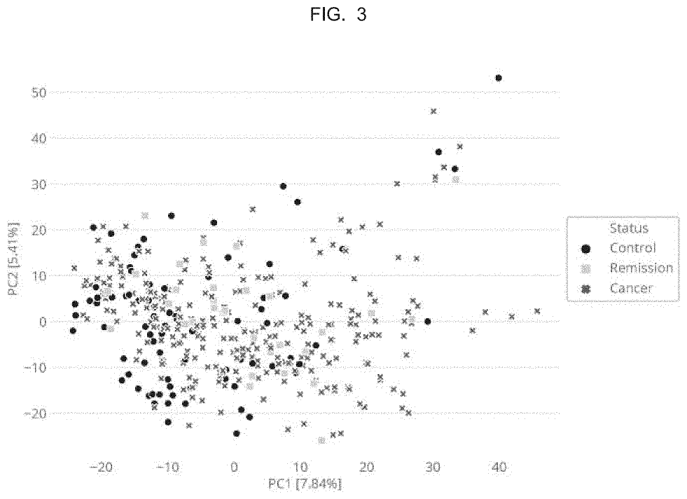

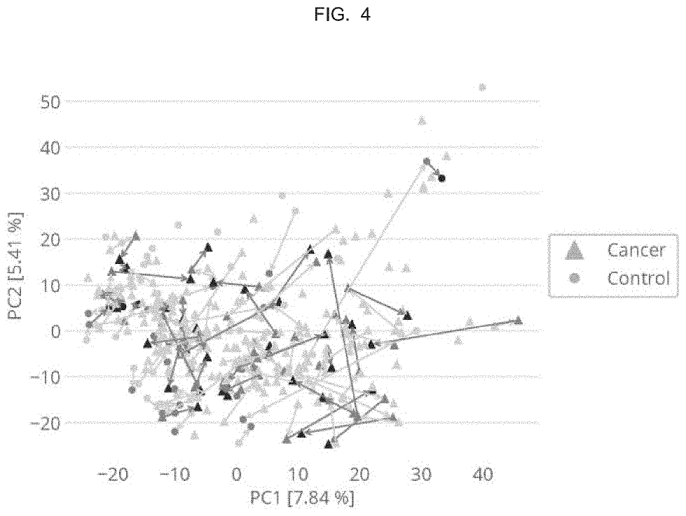

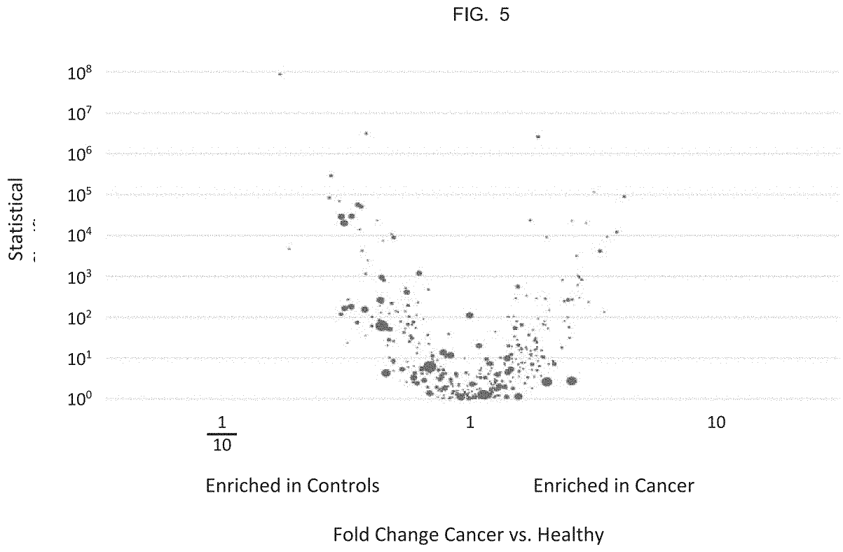

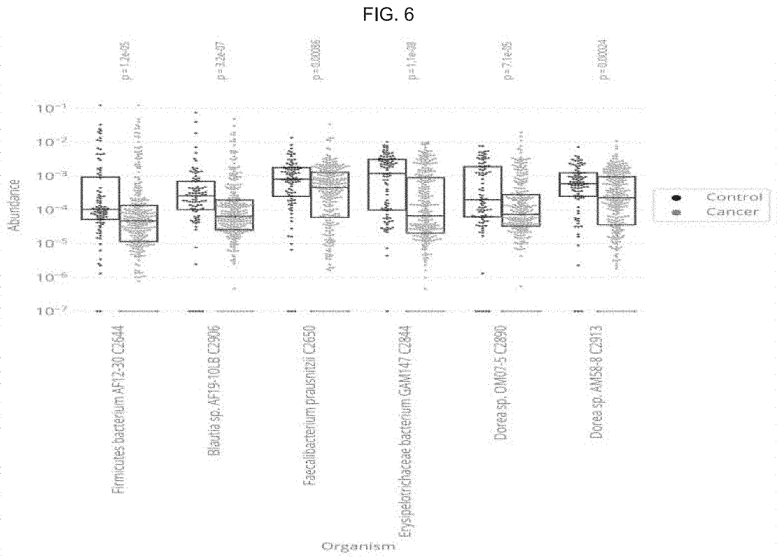

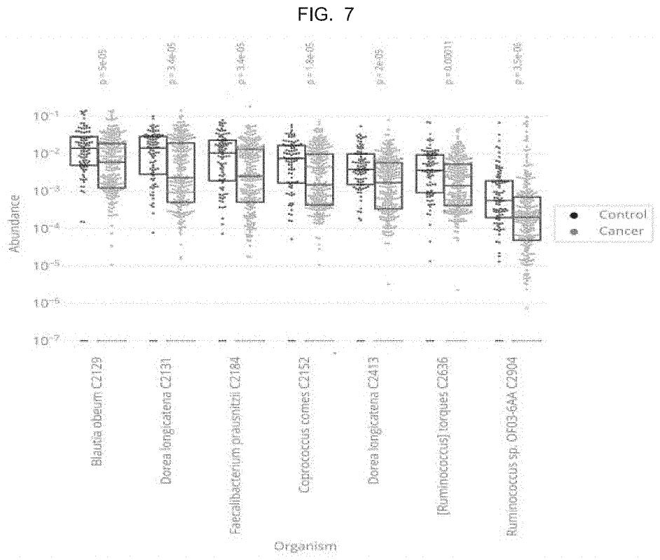

The results were analyzed for differential relative abundance of organisms (classified as OSUs) between cancer and control cohorts, as well as correlations between relative abundance of organisms and immune markers, as measured by flow cytometry. Principal component analysis was performed to visualize the structure of the data (FIG. 3 and FIG. 4) and exhibited a partial separation between cancer and control samples. This separation is driven by a specific subset of microbes that have differential abundance between the two cohorts (FIGS. 5-7 and Table 3). Microbes were ranked based on the magnitude and significance of this difference. Additionally, machine learning was performed to train a model capable of discriminating between a subject with cancer and a control subject.

### Identification of Viral Genetic Material in Stool or Blood Samples

The DNA extracted from stool samples is also used to determine presence of viral DNA material in the stool. Using the sequencing information obtained above, reads not mapping to human or bacterial DNA are aligned to a viral sequence database, for example the NCBI viral genomes database (https://www.ncbi.nlm.nih.gov/genome/viruses/). To detect RNA viruses, a separate sequencing run is required. RNA is extracted from the stool sample by binding to an RNAEASY™ (RNeasy™) column (Qiagen) followed by washing and elution using the reagents provided in the RNeasy™ kit (Qiagen). Sequencing libraries are prepared from RNA by fragmentation, ribodepletion, cDNA synthesis, PCR amplification, and barcoding as described in the TRUSEQ® mRNA sample preparation kit (Illumina). Sequencing analysis is conducted on the Illumina platform using paired-end 150 bp reads. Reads not mapping to human or bacterial DNA are then aligned to a viral sequence database, for example the NCBI viral genomes database (https://www.ncbi.nlm.nih.gov/genome/viruses/). Both of these approaches will provide the identity and relative quantity (for example, viral reads per total reads) of the virus. An analogous procedure is used to identify viral DNA or RNA in blood samples.

Metagenomic sequences are also scanned to identify novel CRISPR sequences using a scoring algorithm such as that described in (Moreno-Mateos et al. (2015) Nat. Met. 12:982-988), and for predicted natural product gene clusters using the ANTISMASH™ (antiSMASH™) routine (Medema et al. (2011) Nuc. Acids Res. 39:W339-W346).

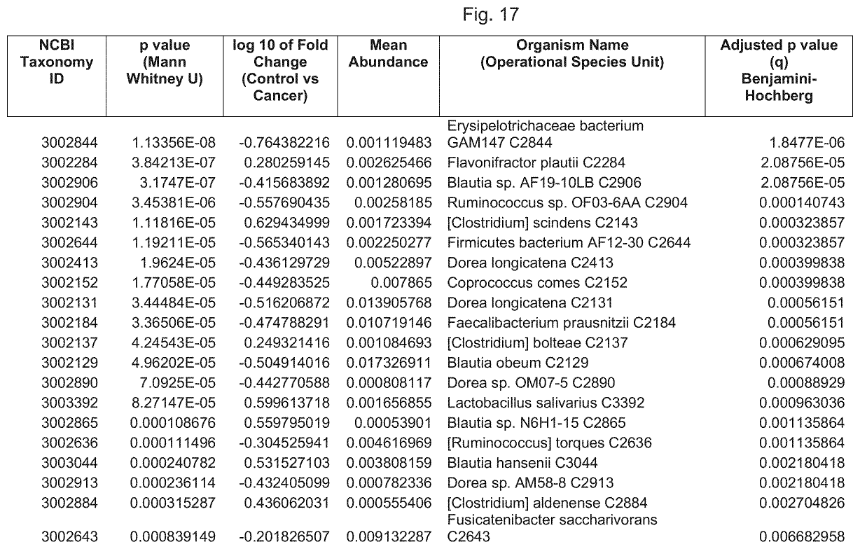

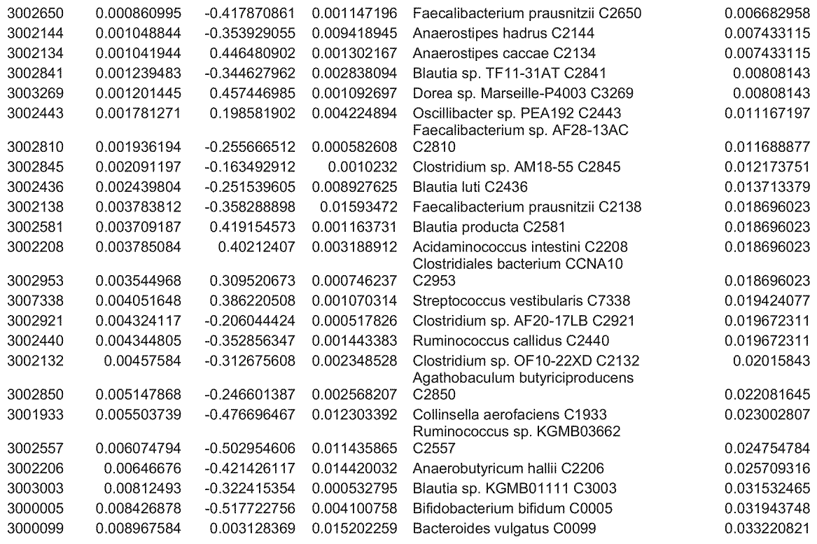

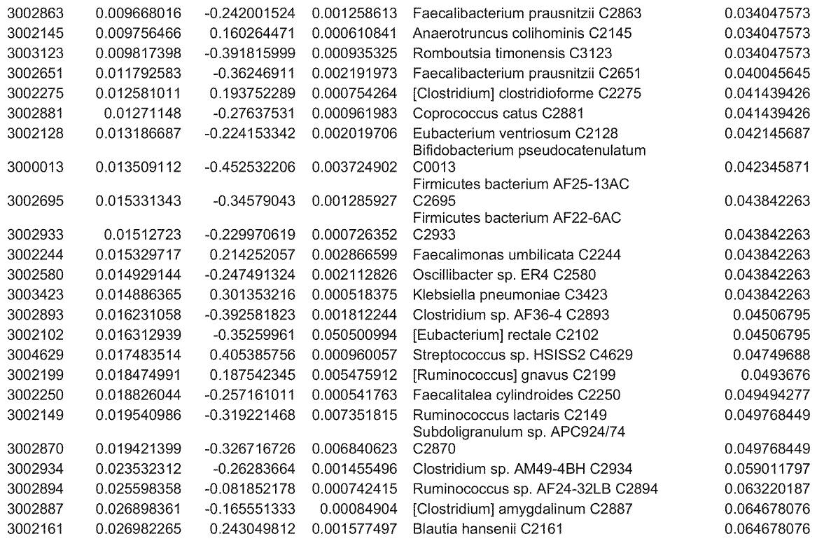

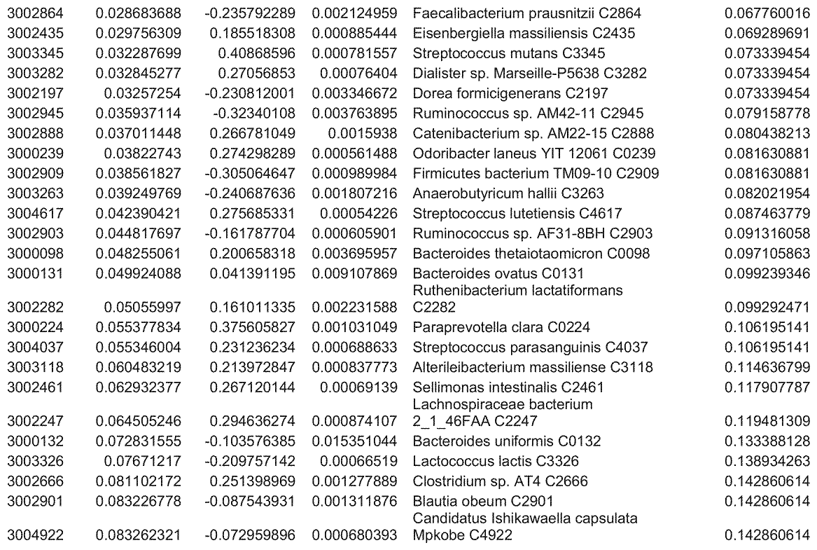

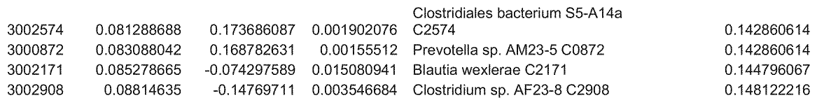

Table 3, illustrated as FIG. 17. Whole genome sequencing was performed on fecal samples from subjects with and without cancer and the reads were classified and abundance of each operational species unit (OSU) was estimated computationally. The fold change difference and statistical significance (inverse p value, Mann Whitney U test) was calculated for abundances between cancer and control sample cohorts. For OSUs with a mean relative abundance of at least 0.05%, p-values were filtered using an adjusted p-value computed using a two-stage Benjamini-Hochberg procedure. OSUs passing the threshold are reported.
Flow Cytometry Analysis of Peripheral Blood from Cancer Patients

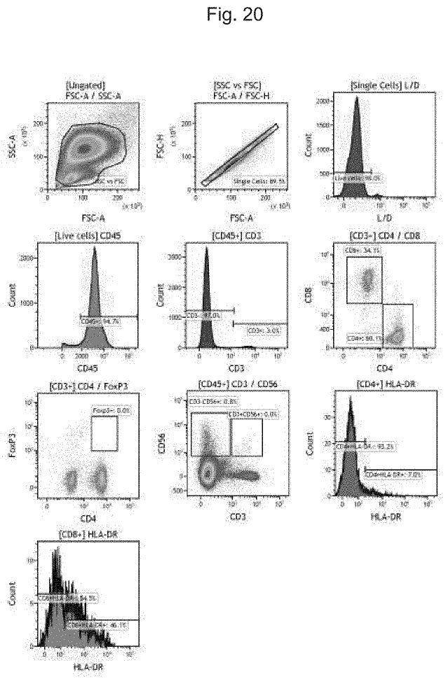

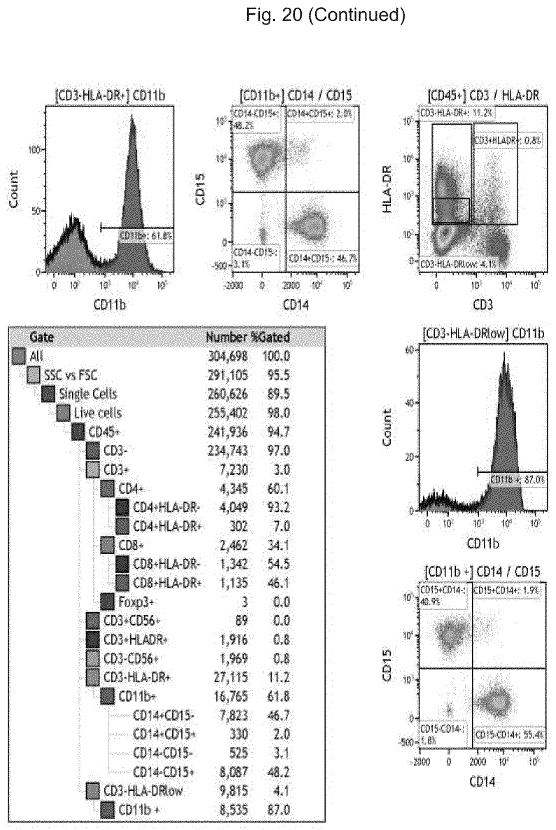

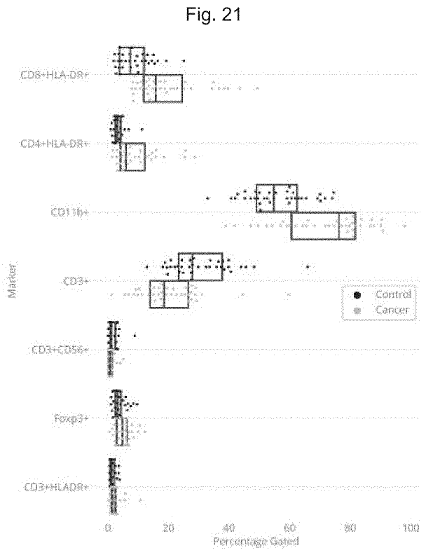

Flow cytometry analysis of peripheral blood can provide a non-invasive immune profile of the patients on study (Showe et al. Cancer Res. 2009 Dec. 15; 69(24): 9202-9210). The peripheral blood immuno-profile evaluation was performed on blood samples collected prior to and after the dosing with the immunotherapy. Phenotypic markers of lymphocyte subpopulations and regulatory T cells (Tregs) was evaluated using flow cytometry with populations gated to include CD3, CD4, CD8, CD25, CD45 and FoxP3-expressing cells using antibodies to each cell type (BD Biosciences). Peripheral blood cells are stained with Live/Dead violet dye (Invitrogen, Carlsbad, Calif.) to gate on live cells. Data is acquired on an LSR II™ flow cytometer (BD Biosciences) and analyzed with FLOWJO™ software (TreeStar, Ashland, Oreg.). Exemplary flow cytometry analysis of peripheral blood samples from a patient undergoing immunotherapy are shown in FIG. 20.

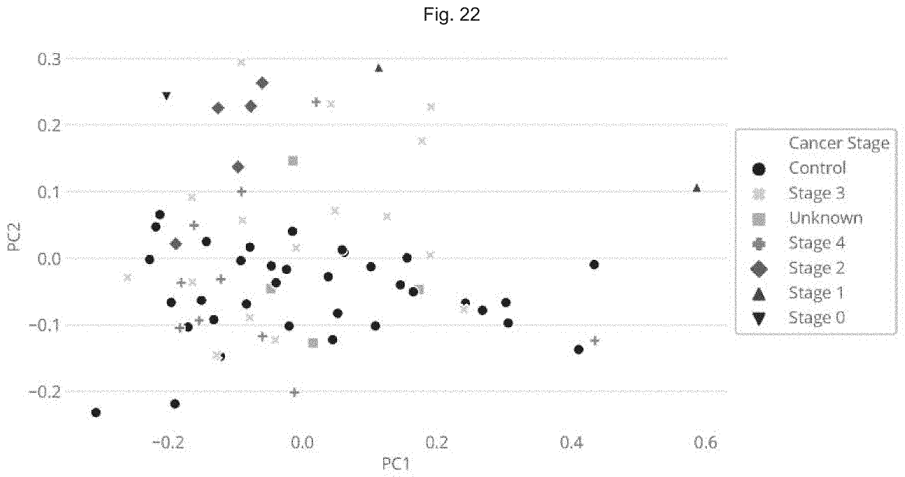

Flow cytometry was performed on 73 blood samples obtained from human subjects with and without cancer. The resulting gated percentages are plotted for different cell markers. For CD8+HLA-DR+, CD4+HLA-DR+, CD11b+, CD3+, CD3+CD56+, Foxp3+, and CD3+HLA-DR+, statistically differences are observed between the cancer and non-cancer populations as shown in FIG. 21. CD8+HLA-DR+(activated cytotoxic T cells) and CD4+HLA-DR+(activated T helper cells) are enriched in the cancer population. Similar immune responses have been noted with patients with COVID-19 prior to symptomatic recovery (Thevarajan et al. (2020), Nature Med. https://doi.org/10.1038/s41591-020-0819-2). P values are computed using the Mann-Whitney U test. Principal component analysis was also conducted on the same data set where the gated percentages are mean and standard deviation scaled. The first two principal components are plotted as shown in FIG. 22. A statistically significant difference is observed between the cancer and control populations in the scaled data. The P value is computed using permutational multivariate analysis of variance (PERMANOVA).

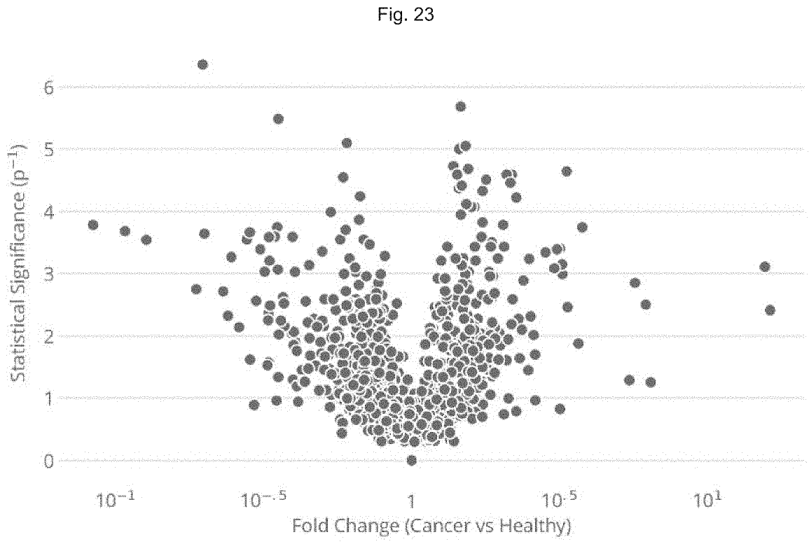

FIG. 23 illustrates metabolomics data on plasma from a third-party provider was processed using a Mann Whitney U test to find significantly different metabolites between cancer and control cohorts. Metabolites enriched in cancer samples appear on the right and those enriched in control samples occur on the left, with higher points on the y-axis corresponding to increased statistical significance.

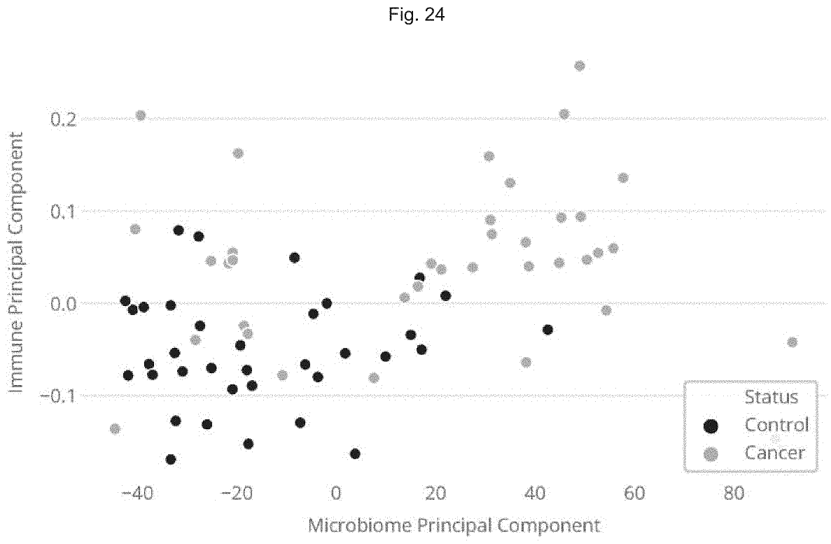

FIG. 24 illustrates the primary principal components for the microbiome sequencing data and immune flow cytometry data are plotted against each, revealing a strong correlation and suggesting that the microbiome may play a role in affecting the immune system and vice versa. Similarly, the interaction between the microbiome and the immune system has been shown to be critical in regulating immune defense against respiratory tract influenza A virus infection (T. Ichinohe et al., Proc. Natl. Acad. Sci. U.S.A 108, 5354-9 (2011) and immunity to vaccines (Hagan et al. (2019) Cell 178 (6): 1313-1328).

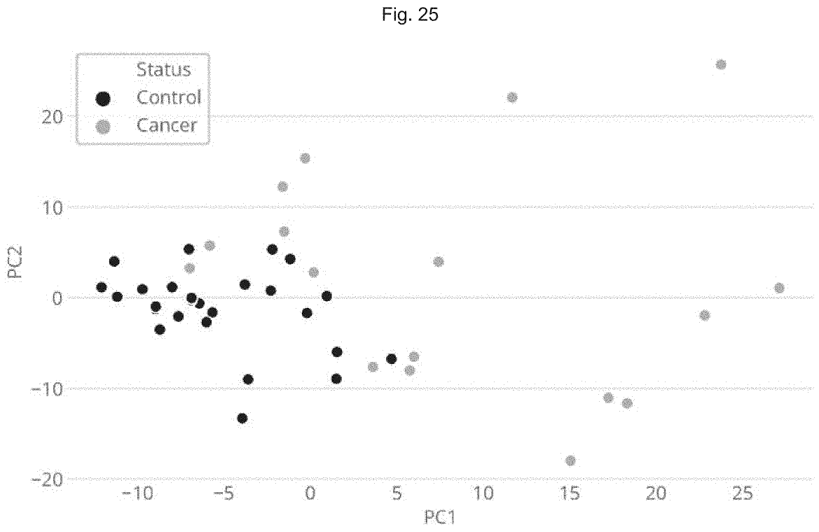

FIG. 25 illustrates metabolomics data on plasma from a third-party provider was processed using a log transform and PCA to show clear separation between samples from a cancer and control cohort.

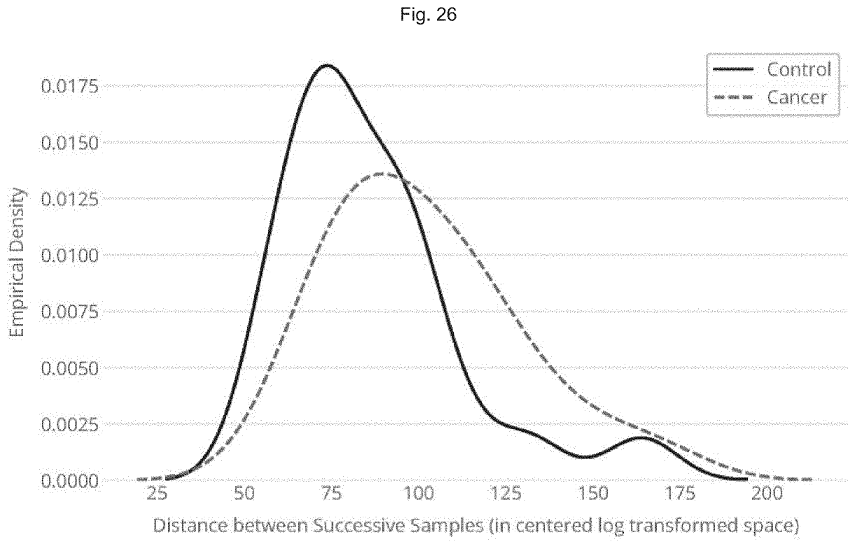

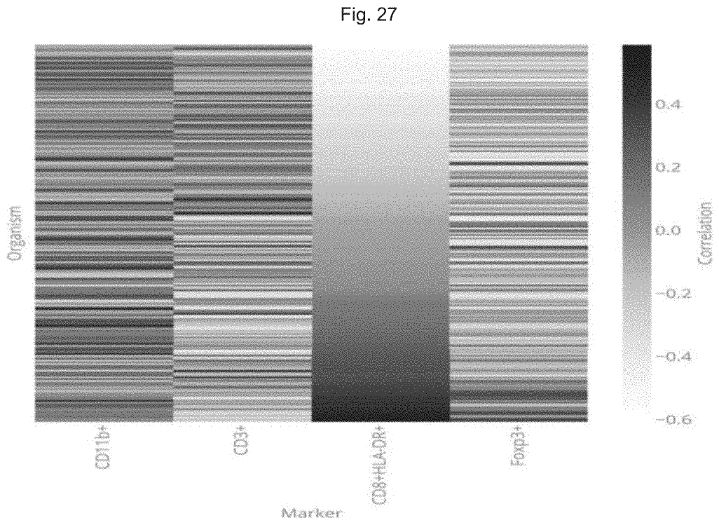

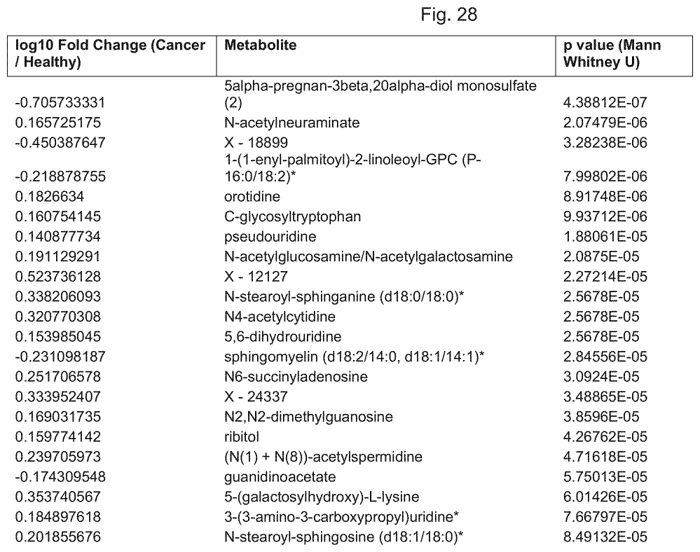

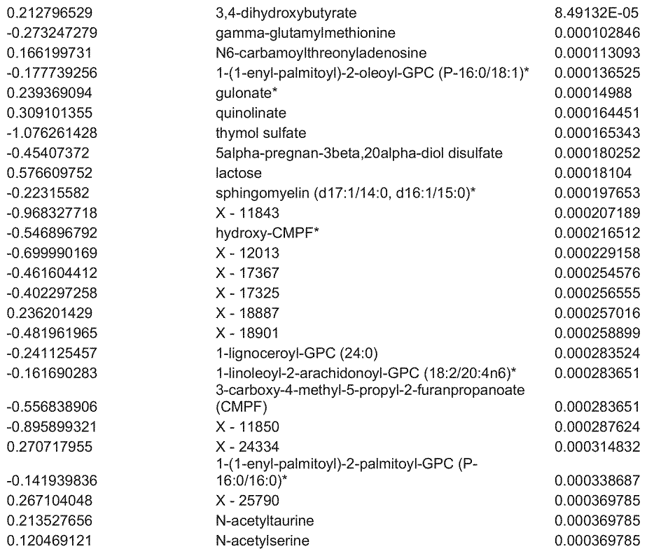

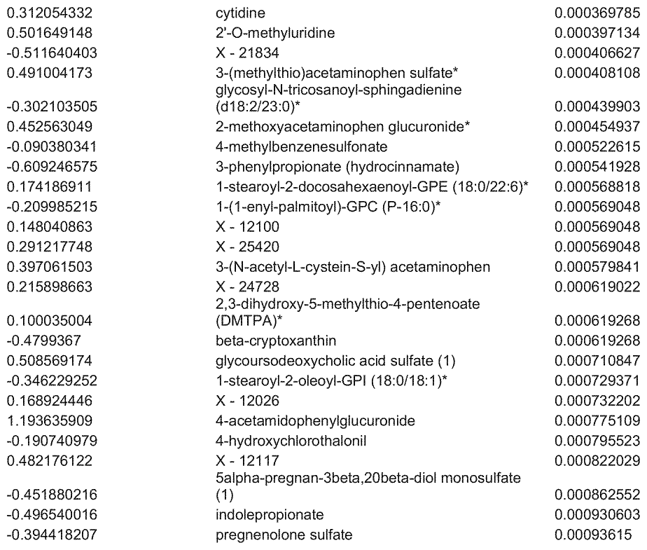

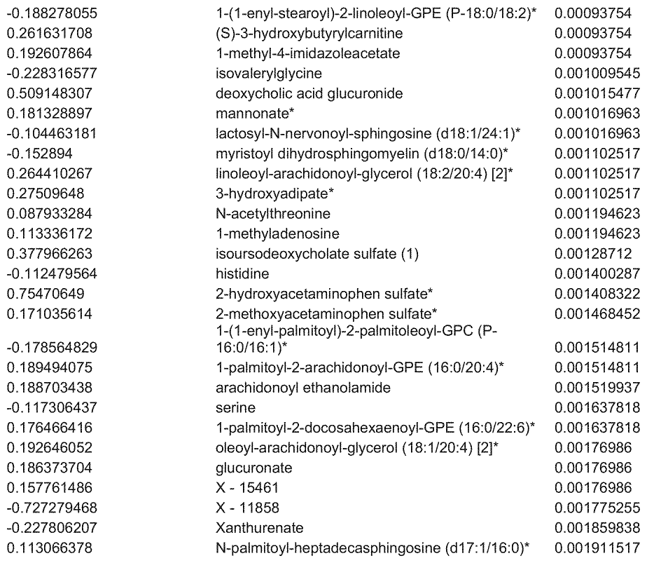

The empirical distribution between successive longitudinal samples is plotted in FIG. 26 for both cancer and control cohorts, demonstrating the increased variability of the cancer microbiome. In FIG. 26, centered log transformed estimated species abundances were generated for both cancer and control sample cohorts. Distances between successive longitudinal samples in the transformed space were computed for both cancer and control cohorts, and the empirical densities of the distances are displayed, revealing that cancer microbiomes are less stable and move around more over time than control.

Spearman correlations were calculated from the peripheral blood flow cytometry analyses and microbiome whole genome sequencing results. Spearman correlations were calculated between each flow gate for humans and each organism in the gut whose mean abundance is greater than or equal to 0.0005. Spearman correlations were calculated between each flow gate (CD11b+, CD3+, CD8-HLADR+ and FoxP3+) for humans and each organism in the gut whose mean abundance is greater than or equal to 0.0005. Results are plotted in a heat map fashion as reported in FIG. 427.

### Metabolomics Analysis of Patient Fecal and Blood

Commensal microbiota metabolites have been shown to be critical in suppressing influenza virus as well as the replication of herpes simplex virus (HSV)-2 (N. Li, et. al. Front. Immunol. 10 (2019), p. 1551). The results described here were obtained from a study involving cancer patients undergoing immunotherapy treatment and healthy controls. Metabolites elucidated as being absent in patients not responding well to treatment are relevant for the treatment of viral infections because in both cases a healthy immune response is required to combat the disease. Therefore, it is reasonable to expect that microbial metabolites beneficial for immuno-oncology treatment will also be beneficial or even essential for rapid viral clearance.

Metabolomics was performed on fecal samples taken from eight cancer patients and two healthy individuals. A total of 856 metabolites could be identified in one or more ofthese samples.

Here we look at all metabolites that were significantly increased in the cancer patients relative to the healthy controls, based on Welch's two-sample t-test with p<0.05, see Tables 11 and 12:

TABLE 11
List of metabolites increased in the cancer population relative
to the control group, given as the ratio of the mean peak
areas for the specified metabolites. Significance was evaluated
based on Welch's two-sample t-test with p < 0.05.

| Compound | Ratio cancer/control | P value |
|---|---|---|
| tyramine | 566 | 0.00415 |
| Taurine | 278 | 0.00390 |
| creatinine | 274 | 0.0230 |
| Indolelactate | 97.6 | 0.0537 |
| OAHSA (T8:1/OH-18:0) | 92.5 | 0.00853 |
| Arachidonic acid (20:4n6) | 86.5 | 0.00836 |
| LAHSA (18:2/OH-18:0)* | 73.9 | 0.00797 |
| Alpha-hydroxyisovalerate | 55.0 | 0.0182 |
| docosahexaenoate (DHA; 22:6n3) | 47.2 | 0.0176 |
| docosahexaenoate (DHA; 22:6n3) | 41.0 | 0.0359 |
| sulfate | 30.7 | 0.0113 |
| 2-hydroxypalmitate | 30.4 | 0.0429 |
| stachydrine | 25.4 | 9.56E−5 |
| Cholate sulfate | 25.2 | 0.0317 |
| Palmitoylcarnitine (C16) | 24.6 | 0.0139 |
| phenethyl amine | 21.5 | 0.0223 |
| N-propionylmethionine | 20.6 | 0.00669 |
| dihydroferulate | 20.0 | 0.0120 |
| Beta-alanine | 19.6 | 0.0145 |
| tryptamine | 19.5 | 0.0289 |
| 3-ureidopropionate | 18.7 | 0.00232 |
| Stearoylcarnitine (C18) | 17.7 | 0.00365 |
| 2-hydroxybutyrate | 17.5 | 0.00802 |
| 3-methylhistidine | 15.5 | 0.0331 |
| Nervonate (24:1n9) | 14.8 | 0.0278 |
| 1-palmitoyl-2-oleoyl-GPE (16:0/18:1) | 14.5 | 0.0281 |
| 5,6-dihydrothymine | 11.8 | 0.0294 |
| octadecadienedioate (C18:2-DC) | 11.2 | 0.0299 |
| agmatine | 10.8 | 0.0428 |
| caffeine | 10.0 | 0.0268 |
| N-methylhydantoin | 9.8 | 0.0405 |
| gentisate | 9.6 | 0.0121 |
| ceramide (d18:2/24:1, d18:1/24:2) | 8.9 | 0.0292 |
| homostachydrine | 8.3 | 0.00739 |
| N-acetylvaline | 8.3 | 0.00242 |
| xanthurenate | 7.9 | 0.0141 |
| N-acetylalanine | 7.4 | 0.0304 |
| Margaroylcamitine (C17) | 7.3 | 0.0256 |
| S-methylcysteine | 6.5 | 0.0449 |
| Hydatoin-5-propionate | 6.3 | 0.0238 |
| N-acetylphenylalanine | 6.3 | 0.0079 |
| N-acetylleucine | 6.0 | 0.00918 |
| Adrenate (22:4n6) | 4.9 | 0.0212 |
| diaminopimelate | 4.3 | 0.0268 |
| pristanate | 4.0 | 0.0331 |
| 2-aminoheptanoate | 3.9 | 0.0296 |
| sarcosine | 3.8 | 0.0380 |
| 2-hydroxyheptanoate | 3.6 | 0.0163 |
| Gamma-glutamylglutamate | 3.6 | 0.0466 |
| lysine | 3.2 | 0.0109 |
| 4-oxovalerate | 3.2 | 0.00970 |
| 3-methyl-2-oxovalerate | 3.2 | 0.0122 |
| Eicosenoylcamitine (C20:1) | 3.1 | 0.0414 |
| 1-methylguanidine | 3.0 | 0.00760 |

TABLE 12
List of metabolites decreased in the cancer population relative
to the control group, given as the ratio of the mean peak
areas for the specified metabolites. Significance was evaluated
based on Welch's two-sample t-test with p < 0.05.

| Compound | Ratio cancer/control | P value |
|---|---|---|
| L-urobilin | 0.07 | 0.00466 |
| Linolenate (18:3n3 or 18:3n6) | 0.11 | 0.0192 |
| Linoleoyl-linolenoyl-glycerol | 0.12 | 0.000537 |
| (18:2/18:3) | | |
| Heptadecatrienoate (17:3) | 0.13 | 0.00224 |
| Heptadecatrienoate (17:3) | 0.13 | 0.00224 |
| Azelate (C9-DC) | 0.13 | 0.0151 |
| Undecanedioate (C11-DC) | 0.14 | 0.0203 |
| Linoleoyl-linolenoyl-glycerol | 0.15 | 0.0348 |
| (18:3/18:3) | | |
| Suberate (C8-DC) | 0.29 | 0.00177 |
| Octadecanedioate (C18-DC) | 0.35 | 0.00999 |
| N-acetylglutamate | 0.43 | 0.0178 |
| Oleoyl-linolenoyl-glycerol (18:1/18:3) | 0.59 | 0.0214 |
| pyridoxamine | 0.60 | 0.0446 |
| 2-oxo-1-pyrrolindinepropionate | 0.75 | 0.0314 |

In a separate study, metabolomics was performed on a total of 55 samples obtained from 22 healthy subjects and 18 cancer patients. In some cases, two or more samples were from the same individual, spaced 6 weeks apart; in such a case they are referred to as timepoints T1 and T2. In general, T1 samples were prior to immunotherapy treatment while T2 samples were during treatment. Approximately 1 gram of raw fecal material stored at −80 deg. C. was processed for metabolite extraction by methanol as described above.

Metabolomics was also performed on plasma extracted from blood obtained from some of the same subjects as the fecal samples. There was a total of 44 plasma samples obtained from 18 healthy subjects and 10 cancer patients. To obtain plasma, 1 mL whole blood was centrifuged at 2800×g for 10 minutes, creating two phases with the plasma on top. 0.5 mL of plasma was removed using a pipette and transferred to a clean tube which was then stored at −80 deg. C. until processing. 0.1 mL of the plasma was used for metabolite extraction, with methanol under vigorous shaking for 2 min (Glen Mills GENOGRINDER 2000™) to precipitate protein and dissociate small molecules bound to protein or trapped in the precipitated protein matrix, followed by centrifugation to recover chemically diverse metabolites. The resulting extract was divided into five fractions: two for analysis by two separate reverse phase (RP)/UPLC-MS/MS methods using positive ion mode electrospray ionization (ESI), one for analysis by RP/UPLC-MS/MS using negative ion mode ESI, one for analysis by HILIC/UPLC-MS/MS using negative ion mode ESI, and one reserved for backup. Samples are placed briefly on a TURBOVAP® (Zymark) to remove the organic solvent. The sample extracts are stored overnight under nitrogen before preparation for analysis.

Three types of controls were analyzed in concert with the experimental samples: a pooled sample generated from a small portion of each experimental sample of interest served as a technical replicate throughout the platform run; extracted water samples served as process blanks; and a cocktail of standards spiked into every analyzed sample allowed for instrument performance monitoring. Instrument variability was determined by calculation of the median relative s.d. (RSD) for the standards that were added to each sample before injection into the mass spectrometers (median RSDs were determined to be 3% for plasma and 4% for fecal extracts). Overall process variability was determined by calculating the median RSD for all endogenous metabolites (i.e., non-instrument standards) present in 90% or more of the pooled technical-replicate samples (median RSD of 7% for plasma and 10% for fecal).

Compounds are identified by comparison to library entries of purified standards maintained by Metabolon, that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library+/−10 ppm, and the MS/MS forward and reverse scores. MS/MS scores are based on a comparison of the ions present in the experimental spectrum to ions present in the library entry spectrum. While there may be similarities between these molecules based on one of these factors, the use of all three data points can be utilized to distinguish and differentiate biochemicals. Peaks are quantified as area-under-the-curve detector ion counts.

A total of 992 known compounds were identified in at least one of the plasma samples, and 1049 were identified in at least one of the fecal samples. 734 of these compounds were common between the two sample types.

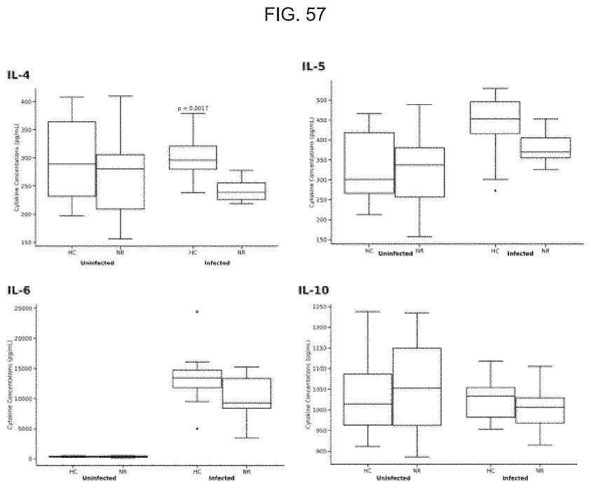

The overall metabolic profiles were represented as two principal components. Principal components analysis is an unsupervised statistical method that compresses the number of dimensions of the data to provide a high-level view of the data over an entire set of samples. Each principal component is a linear combination of every metabolite and the principal components are uncorrelated. Principal components analysis exhibited a reasonable ability to separate the cancer and healthy groups, especially in plasma. When considering two principal components, there was a notable separation of healthy controls from cancer samples collected at T1 or T2 in plasma (FIG. 57, left panel). Interestingly, four samples from three cancer group subjects whose fecal whole metagenomic sequencing data clustered with healthy rather than cancer subjects also clustered on PCAs with healthy subject based on metabolic profiles in plasma. Points corresponding to these samples are indicated in the plots by arrows. In fecal samples, there was much greater overlap of healthy and cancer groups on PCA, though samples from these same cancer patients (labeled 95798, 96218, and PN4) were centered among the greatest concentration of healthy samples (FIG. 57, right panel).

FIG. 28 is a table of the top 100 differential metabolites, ranked by p value (Mann Whitney U test). Metabolomics data on plasma from a third-party provider was processed using a Mann Whitney U test to find significantly different metabolites between cancer and control cohorts. The top 100 metabolites ranked by p value are reported.

Examination of the results demonstrated potential differences between the plasma metabolic phenotype in healthy versus cancer T1 and cancer T2 groups (Table 13). Specifically, compounds connected to pathways of protein degradation (i.e., modified amino acids), chromatin packing in the nucleus (i.e., polyamines), nucleotide metabolism (i.e., pentose phosphate and nucleotide pathways), and extracellular matrix metabolism (i.e., aminosugars) were prioritized for their connection to activities prominent in cancer including proliferation and DNA synthesis, cell division, and invasion. Potential markers of protein post-translational modification and proteolysis (for example, N-acetyl amino acids) were elevated in plasma from both cancer T1 and T2 relative to the healthy group, respectively. Elevated proteinase expression and activity are associated with metastatic cancers (extracellular matrix invasion, autophagy, etc.) and signs of proteinase activity can be registered in the metabolome by the appearance of post-translationally modified amino acids. Likewise, polyamines and nucleic acids are required for the synthesis and packaging of DNA in proliferating cells, and these metabolites tended to be higher at both cancer T1 and T2 with respect to the healthy control group. Glycosaminoglycan degradation and oxidation products (for example, N-acetylneuraminate, the isobar N-acetylglucosamine/N-acetylgalactosamine, erythronate) were moderately elevated in cancer T1 and T2 compared to healthy controls. Reductions in various progestin steroids were noticeable in cancer T1 and T2 compared to the healthy group. Together, these biomarker patterns could reflect a persistent cancer phenotype related to protein degradation, nucleic acid synthesis, turnover, and packaging, extracellular matrix glycan turnover, and altered hormonal regulatory cues.

TABLE 13
Compounds in plasma possibly representative of a cancer phenotype
with statistically significant elevations in either cancer T1, cancer
T2 or both relative to the healthy control group. Values given are
ratios of the mean peak areas for the specified metabolites between
the two groups indicated. Up or down arrows indicate whether the
increase or decrease in the treatment relative to the control is
significant based on Welch's two-sample t-test with p < 0.05.

| | Cancer | Cancer | |
|---|---|---|---|
| | T1/All | T2/All | Cancer T2/ |
| Compound | Healthy | Healthy | Cancer T1 |
| N-acetylserine | 1.41 ↑ | 1.31 | 0.93 |
| N-acetylalanine | 1.24 ↑ | 1.21 | 0.98 |
| Hydroxyasparagine | 1.4 ↑ | 1.32 | 0.94 |
| 5-galactosylhydroxy-L-lysine | 2.3 ↑ | 1.73 ↑ | 0.75 |
| C-glycosyltryptophan | 1.44 ↑ | 1.41 ↑ | 0.98 |
| N-acetylputrescine | 1.65 | 1.22 ↑ | 0.74 |
| N-acetyl-isoputreanine | 1.2 | 1.19 ↑ | 0.98 |
| (N(1) + N(8))-acetylspermidine | 1.9 ↑ | 1.89 ↑ | 1 |
| Acisoga | 1.43 ↑ | 1.35 ↑ | 0.94 |
| 5-methylthioadenosine | 2.01 ↑ | 1.95 ↑ | 0.97 |
| Ribitol | 1.82 ↑ | 1.29 | 0.71 |
| Ribonate | 1.37 ↑ | 1.11 | 0.81 |
| Arabitol/xylitol | 1.48 ↑ | 1.08 | 0.73 |
| Glucuronate | 2.2 ↑ | 1.07 | 0.48 |
| N-acetylneuraminate | 1.47 ↑ | 1.5 ↑ | 1.02 |
| Erythronate | 1.2 ↑ | 1.26 | 1.05 |
| N-acetylglucosamine/N- | 1.52 ↑ | 1.57 ↑ | 1.03 |
| acetylgalactosamine | | | |
| 5-alpha-pregnan-3beta,20beta-diol | 0.18 ↓ | 0.24 ↓ | 1.3 |
| monosulfate (1) | | | |
| 5-alpha-pregnan-3beta,20beta-diol | 0.11 ↓ | 0.14 ↓ | 1.24 |
| monosulfate (2) | | | |
| 5-alpha-pregnan-3beta,20beta-diol | 0.24 ↓ | 0.38 ↓ | 1.19 |
| disulfate | | | |
| 5-alpha-pregnan-diol disulfate | 0.25 ↓ | 0.3 | 1.21 |
| Pregnanediol-3-glucuronide | 0.25 ↓ | 0.23 ↓ | 0.92 |
| Adenine | 1.55 ↑ | 1.5 | 0.97 |
| N1-methyladenosine | 1.29 ↑ | 1.41 ↑ | 1.1 |
| N6-carbamoylthreonyladenosine | 1.5 ↑ | 1.31 | 0.87 |
| N6-succinyladenosine | 1.88 ↑ | 1.82 ↑ | 0.97 |
| 7-methylguanine | 1.29 ↑ | 1.02 | 0.8 ↓ |
| N2,N2-dimethylguanosine | 1.55 ↑ | 1.45 ↑ | 0.94 |
| Orotidine | 1.62 ↑ | 1.54 ↑ | 0.95 |
| Pseudouridine | 1.45 ↑ | 1.38 ↑ | 0.95 |
| | 1.46 ↑ | 1.43 ↑ | 0.98 |
| 2′-O-methyluridine | 2.81 ↑ | 0.46 | 0.16 |
| Cytidine | 2.41 ↑ | 2.25 ↑ | 0.93 |
| N4-acetylcytidine | 2.38 ↑ | 2.1 ↑ | 0.88 |
| 2′-O-methylcytidine | 1.92 ↑ | 1.58 | 0.82 |

The tricarboxylic acid (TCA) cycle and glycolysis pathways connected to energy production from glucose were enriched with connected metabolites that differed significantly between the plasma cancer T1 and cancer T2 groups (Table 14). In cancer the TCA cycle has been noted to serve as both a source of energy production and as a central metabolic node in the utilization and production of key metabolite classes including free fatty acid synthesis from citrate, heme from fumarate, nucleotides and proteins from oxaloacetate and alpha-ketoglutarate [3]. Mutations affecting dysregulation of oncogenes and tumor suppressors have direct impact on TCA cycle metabolism and transport of substrates into the mitochondria and direct mutations of TCA cycle enzymes also occur with some cancers [4]. Although carbon from glucose is presented as the canonical substrate for citrate production, carbons from both fatty acids and amino acids readily enter the cycle at specific points. Glutamine, via glutaminolysis to glutamate, is noted as a highly utilized fuel and carbon source for many cancers [5; 6]. The shifting profile of glutamate, pyruvate, and TCA cycle metabolites in the cancer T2 group relative to the cancer T1 group suggest that anticancer treatment has a disruptive effect on energy or mitochondrial carbon repurposing.

TABLE 14
The tricarboxylic acid (TCA) cycle profile in plasma shifted
in cancer T2 compared to cancer T1 as a possible sign of response
to anticancer treatment. Values given are ratios of the mean
peak areas for the specified metabolites between the two groups
indicated. Up or down arrows indicate whether the increase or
decrease in the treatment relative to the control is significant
based on Welch's two-sample t-test with p < 0.05.

| | Cancer | Cancer | |
|---|---|---|---|
| | T1/All | T2/All | Cancer T2/ |
| Compound | Healthy | Healthy | Cancer T1 |
| Glutamate | 1.29 ↑ | 1.31 | 1.01 |
| Pyruvate | 0.93 | 0.68 | 0.73 ↓ |
| Lactate | 1.12 | 0.81 | 0.72 ↓ |
| Citrate | 1 | 1.1 | 1.09 ↑ |
| Isocitric lactone | 1.37 | 2.01 | 1.47 ↑ |
| Alpha-ketoglutarate | 1.11 | 1.21 | 1.09 ↑ |
| Succinate | 1.08 | 0.93 | 0.86 ↓ |
| Fumarate | 0.91 | 0.85 | 0.93 ↓ |
| Malate | 0.97 | 0.91 | 0.94 ↓ |

Plasma metabolites connected to glutathione metabolism and oxidative stress differed in the cancer T2 group with respect to the cancer T1 group (Table 15). Oxidized forms of glutathione and cysteine were reduced in the cancer T2 group relative to the cancer T1 group and may suggest a relative decrease in oxidative stress in the cancer T2 plasma samples. Oxidized ascorbic acid derivatives showed significant reductions in the cancer T2 group compared to the healthy control group. Tumors operate with a high level of incidental oxidative stress through the production of free radicals, reactive oxygen and nitrogen species, and hydrogen peroxide and thus depend on antioxidants such as glutathione and ascorbate to neutralize oxidative species and repair oxidative damage [7; 8]. The decreasing level of oxidative intermediates of glutathione, cysteine, and ascorbate in the cancer T2 group may be a sign of overall reduced metabolic activity and oxidative species production in response to anticancer treatment.

TABLE 15
Most oxidized forms of cysteine, glutathione, and ascorbate in plasma
decreased during anticancer treatment in the cancer T2 group. Values
given are ratios of the mean peak areas for the specified metabolites
between the two groups indicated. Up or down arrows indicate whether
the increase or decrease in the treatment relative to the control is
significant based on Welch's two-sample t-test with p < 0.05.

| | Cancer | Cancer | |
|---|---|---|---|
| | T1/All | T2/All | Cancer T2/ |
| Compound | Healthy | Healthy | Cancer T1 |
| Glycine | 0.79 ↓ | 0.72 ↓ | 0.9 |
| Glutamate | 1.29 ↑ | 1.31 | 1.01 |
| Methionine | 0.79 | 0.8 | 1.02 |
| cysteine | 1.04 | 0.97 | 0.93 ↓ |
| Cystine | 1.25 | 1.63 ↑ | 1.31 |
| Cysteine sulfinic acid | 1.04 | 0.81 | 0.78 ↓ |
| Cysteine-glutathione disulfide | 1.03 | 0.66 | 0.64 |
| Cysteinylglycine | 1.21 | 0.62 | 0.51 ↓ |
| Cysteinylglycine disulfide | 1.14 | 0.89 | 0.78 ↓ |
| Cys-Gly, oxidized | 1.15 | 0.56 | 0.49 ↓ |
| Ascorbic acid 3-sulfate | 1.55 | 0.5 ↓ | 0.32 |
| Threonate | 0.79 | 0.46 ↓ | 0.58 |
| Oxalate | 0.76 | 0.56 ↓ | 0.74 |
| Gul onate | 2.17 ↑ | 1.28 | 0.59 |

Some statistically significant differences in fecal primary and secondary acids were observed for the cancer T2 group with respect to the cancer T1 group (Table 16). Most bile acids in the cancer T1 and cancer T2 groups showed large fold-change differences with respect to the healthy control group but the combination of low statistical power and large within-group variation prevented many of these differences from reaching statistical significance. Primary bile acids produced in the liver serve as emulsifiers to aid nutrient absorption from the digestive tract and are transformed into secondary bile acids by members of the gut microbiota. The significantly altered levels of some primary and secondary bile acids in the cancer T2 group relative to the baseline cancer T1 could reflect altered liver synthesis of primary bile acids, modified systemic transport, or changes in gut microflora composition and bile acid metabolism secondary to the anticancer treatment.

TABLE 16
Altered levels of primary and secondary bile acids in feces
among the sample groups. Values given are ratios of the mean
peak areas for the specified metabolites between the two groups
indicated. Up or down arrows indicate whether the increase
or decrease in the treatment relative to the control is significant
based on Welch's two-sample t-test with p < 0.05.

| | Cancer | Cancer | |
|---|---|---|---|
| | T1/All | T2/All | Cancer T2/ |
| Compound | Healthy | Healthy | Cancer T1 |
| Cholate | 1.07 | 3.28 | 3.07 |
| Glycocholate | 4.5 | 1.52 | 0.34 |
| Taurocholate | 15.98 | 11.5 | 0.72 |
| Chenodeoxycholate | 1.83 | 4.47 | 2.45 |
| Chenodeoxycholic acid (1) | 3.44 ↑ | 2.72 | 0.79 |
| Chenodeoxycholic acid (1) | 1.55 | 5.62 | 3.63 |
| Glycochenodeoxycholate | 3.41 | 1.29 | 0.38 |
| Taurochenodeoxycholate | 8.62 | 3.54 | 0.41 ↓ |
| Cholate sulfate | 2 | 5.74 | 2.86 ↑ |
| Glycochenodeoxycholate 3-sulfate | 17.41 | 1.29 | 0.07 |
| Glycocholate sulfate | 2.85 | 1 | 0.35 ↓ |
| Deoxycholate | 1.27 | 1.56 | 1.23 |
| Deoxycholic acid 3-sulfate | 3.83 | 6.56 | 1.71 |
| Deoxycholic acid (12 or 24)-sulfate | 8.23 ↑ | 4.25 | 0.52 |
| Deoxycholic acid glucuronide | 0.48 | 0.33 | 0.69 ↓ |
| Taurodeoxycholate | 15.78 ↑ | 16.4 | 1.04 |
| Lithocholate | 1.17 | 1.03 | 0.88 ↓ |
| Lithocholate sulfate (1) | 3.58 ↑ | 1.74 | 0.48 |
| Lithocholate sulfate (2) | 4.33 | 6.12 | 1.41 |
| Glycolithocholate sulfate | 2.23 | 1.86 | 0.83 |
| Taurolithocholate 3-sulfate | 2.5 | 2.54 ↑ | 1.01 |
| Ursodeoxycholate | 1.48 ↑ | 2.72 | 1.84 |
| Isoursodeoxycholate | 2.13 | 2.1 | 0.98 |
| Isoursodeoxycholate sulfate (1) | 3.89 ↑ | 5.62 | 1.45 |
| Glycoursodeoxycholate | 2.59 | 1.12 | 0.43 |
| Tauroursodeoxycholate | 2.84 | 1.26 | 0.44 ↓ |
| Taurochenodeoxycholic acid 3-sulfate | 10.04 | 1.13 | 0.11 |
| Ursodeoxycholate sulfate (1) | 2.76 | 11.72 | 4.24 |

Several fecal metabolites with metabolic origins possibly connected to the microbiome were altered in either the cancer T1 or cancer T2 groups compared to the healthy control group (Table 17). These included polyamine compounds such as cadaverine and putrescine, derivatives of the aromatic amino acids—phenylalanine, tyrosine, and tryptophan, benzoates, and compounds related to the microbial-aided breakdown of complex polymers such as lignin present in plant foodstuffs. Many differential changes were apparent between cancer T1 and the healthy group relative to the cancer T2 and healthy group comparison, and other compounds differed in the baseline cancer T1 to cancer T2 treatment groups. The differential pattern of microbiome-associated metabolites in the cancer T1 and cancer T2 groups could reflect compositional changes in the microflora both driven by cancer (i.e., cancer T1 differences) as well as anticancer treatment (i.e., cancer T2 distinctions). A healthy microflora maintains an intestinal barrier that keeps out genotoxic and inflammatory bacteria and their toxins [9]. An increasing number of publications point to likely contributions of dysbiosis and toxins to carcinogenesis and the role of a healthy microflora supported by lifestyle, diet, prebiotics, and probiotics to prevent and serve as anticancer adjuvants are being explored [10].

TABLE 17
Microbiome-associated compounds displayed differential patterns in
the fecal metabolome of the cancer T1 and cancer T2 groups. Values
given are ratios of the mean peak areas for the specified metabolites
between the two groups indicated. Up or down arrows indicate whether
the increase or decrease in the treatment relative to the control
is significant based on Welch's two-sample t-test with p < 0.05.

| | Cancer | Cancer | |
|---|---|---|---|
| | T1/All | T2/All | Cancer T2/ |
| Compound | Healthy | Healthy | Cancer T1 |
| Cadaverine | 1.85 | 3.91 ↑ | 2.11 |
| N-acetyl-cadaverine | 5.06 | 5.56 ↑ | 1.1 |
| Phenethylamine | 0.73 | 1.42 ↑ | 1.95 |
| Tyramine | 2.26 ↑ | 12.82 | 5.69 |
| Phenol sulfate | 6.03 ↑ | 2.12 | 0.35 |
| p-cresol glucuronide | 2.56 | 1 | 0.39 ↓ |
| Vanillic alcohol sulfate | 1 | 35.29 | 35.29 ↑ |
| Tryptamine | 4.79 ↑ | 12.5 | 2.61 |
| Skatol | 1.41 | 0.13 ↓ | 0.09 |
| Indole | 2.63 | 0.89 ↓ | 0.34 |
| Indole-3-carboxylate | 0.83 | 0.26 ↓ | 0.31 |
| 2-aminophenol | 2.82 ↑ | 0.95 | 0.34 |
| Agmatine | 2.67 | 1.92 | 0.72 ↓ |
| Putrescine | 2.18 | 4.89 ↑ | 2.24 |
| N-acetylputrescine | 2.39 | 2.67 | 1.12 |
| Spermidine | 1.16 | 2.37 | 2.04 |
| N(′1)-acetylspermidine | 1.46 | 1.42 ↑ | 0.97 |
| Acisoga | 2.26 ↑ | 1.46 | 0.64 |
| Alpha-CEHC sulfate | 4.5 ↑ | 6.95 | 1.54 |
| Delta-CEHC | 0.78 | 0.56 | 0.73 |
| Gamma-CEHC sulfate | 1.53 ↑ | 3.58 | 2.34 |
| 3-hydroxyhippurate | 0.49 | 0.15 ↓ | 0.31 |
| 2-(4-hydroxyphenyl)propionate | 1.52 | 0.23 ↓ | 0.15 |
| 4-hydroxycyclohexylcarboxylic acid | 0.5 ↓ | 0.94 | 1.89 |
| Caffeate | 0.54 | 0.57 | 1.05 |
| Coumaroylquinate (1) | 0.35 | 0.42 | 1.2 ↑ |
| Coumaroylquinate (3) | 0.54 | 0.58 | 1.08 ↑ |
| Genistein sulfate | 15.6 | 2.23 | 0.14 ↓ |
| Enterolactone | 1.1 ↑ | 0.47 | 0.43 |

Hemne degradation markers, including bilirubin and L-urobilinogen, showed changes across the cancer T1 and cancer T2 compared to the healthy group in feces and in the cancer T1 group of plasma compared to the healthy controls (Tables 18 and 19). Urobilinogen and urobilin are downstream products connected to the microbiome. An interesting recent metabolomic publication found increasing fecal levels of urobilinogen with increasing radiation dose and cross-omic analysis showed that the increase was positively correlated to microbes of the Lachnospiraceae, Ruminococcaceae, and Rikenellacea taxa [11]. This work shows how cross-omic integration can lead to a greater understanding and provide needed specificity to changes in distinct metabolites.

TABLE 18
Heme degradation markers with altered levels in feces. Values given
are ratios of the mean peak areas for the specified metabolites between
the two groups indicated. Up or down arrows indicate whether the
increase or decrease in the treatment relative to the control is
significant based on Welch's two-sample t-test with p < 0.05.

| | Cancer | Cancer | |
|---|---|---|---|
| | T1/All | T2/All | Cancer T2/ |
| Compound | Healthy | Healthy | Cancer T1 |
| Protoporphyrin IX | 1.32 | 0.86 | 0.65 ↓ |
| Bilirubin (Z,Z) | 4.39 ↑ | 2.95 | 0.67 |
| Bilirubin (E,E) | 3.54 | 1.81 | 0.51 |
| Biliverdin | 1.8 | 0.86 | 0.48 |
| Urobilinogen | 3.74 | 5.02 ↑ | 1.34 |
| D-urobilin | 0.99 | 0.73 | 0.74 |
| L-urobilin | 0.37 ↓ | 0.7 | 1.9 |

TABLE 19
Heme degradation markers with altered levels in plasma. Values given
are ratios of the mean peak areas for the specified metabolites between
the two groups indicated. Up or down arrows indicate whether the
increase or decrease in the treatment relative to the control is
significant based on Welch's two-sample t-test with p < 0.05.

| | Cancer | Cancer | |
|---|---|---|---|
| | T1/All | T2/All | Cancer T2/ |
| Compound | Healthy | Healthy | Cancer T1 |
| Heme | 1.15 | 1.98 | 1.72 |
| Bilirubin (Z,Z) | 0.68 ↓ | 0.71 | 1.05 |
| Bilirubin (E,Z) or (Z,E) | 0.66 ↓ | 0.66 | 1 |
| Biliverdin | 0.77 | 0.87 | 1.12 |
| Urobilinogen | 1.72 ↑ | 1.3 | 0.75 |

### Example 10: Data Driven and Machine Learning Approaches for Therapeutic Design

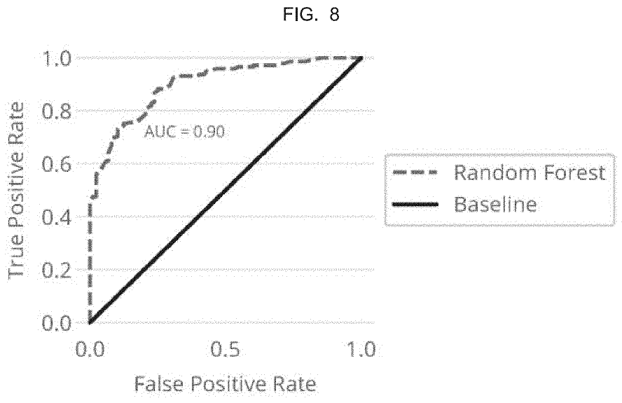

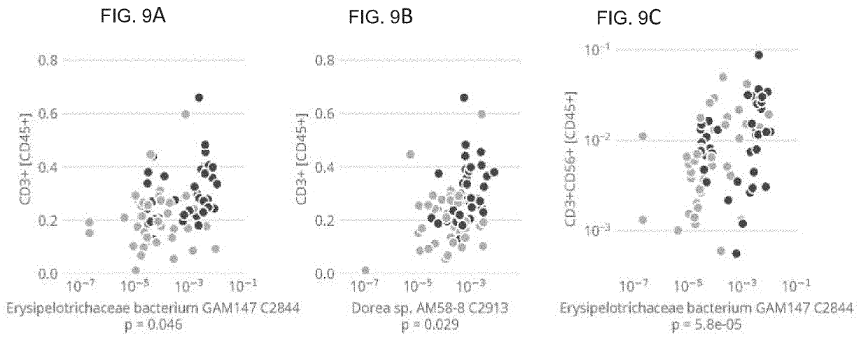

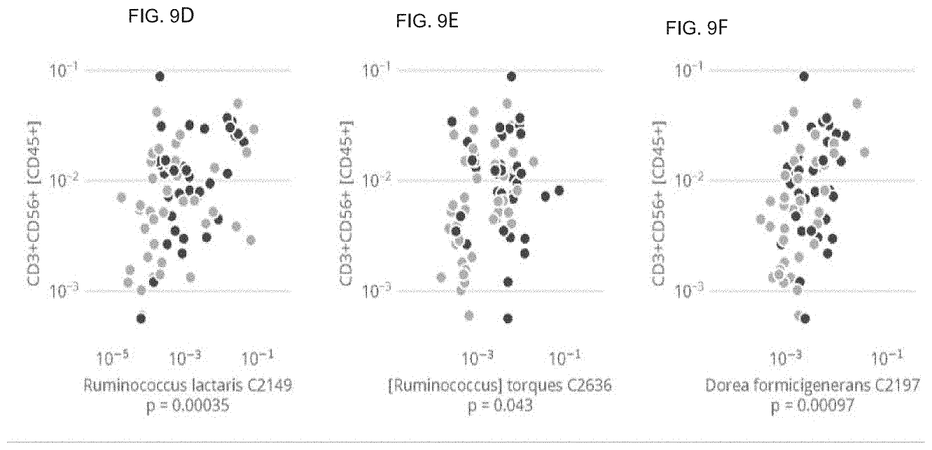

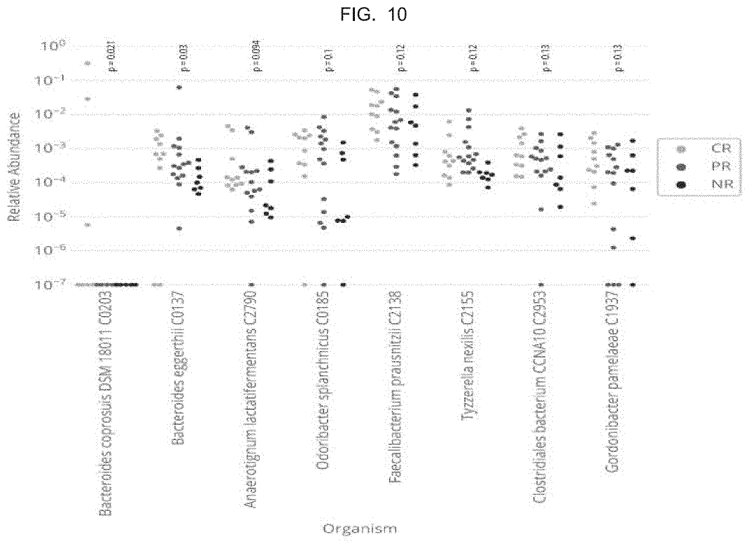

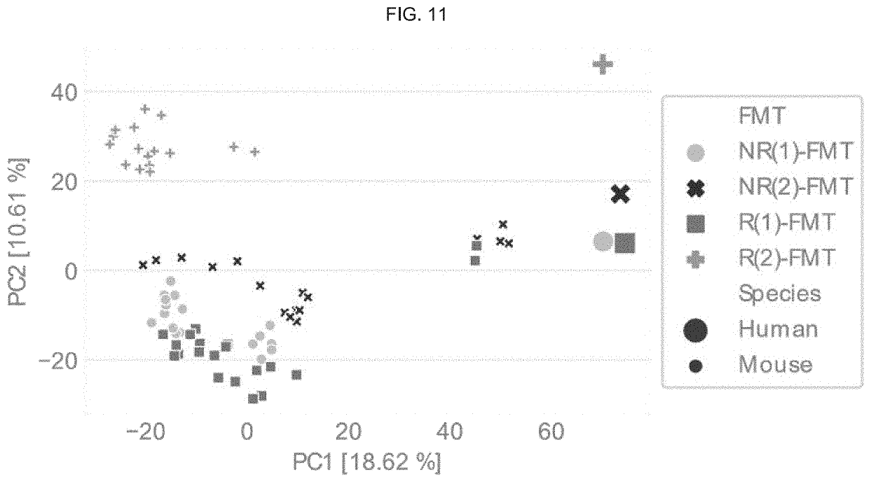

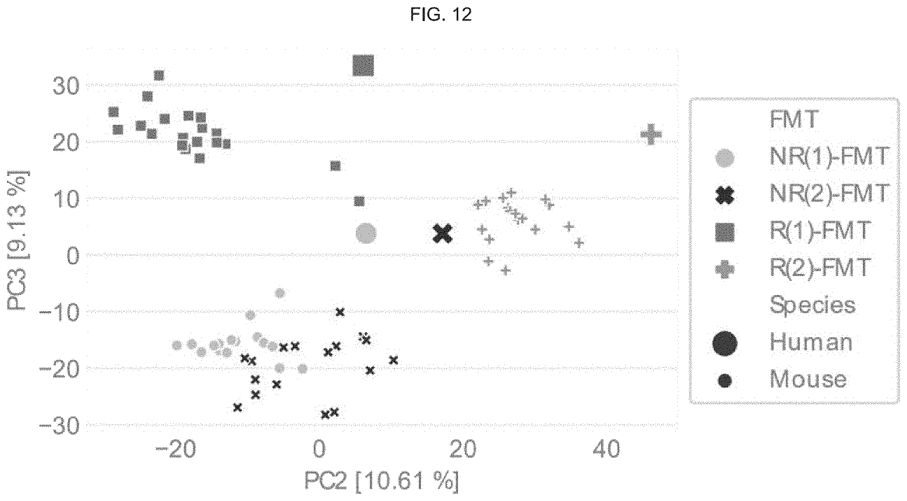

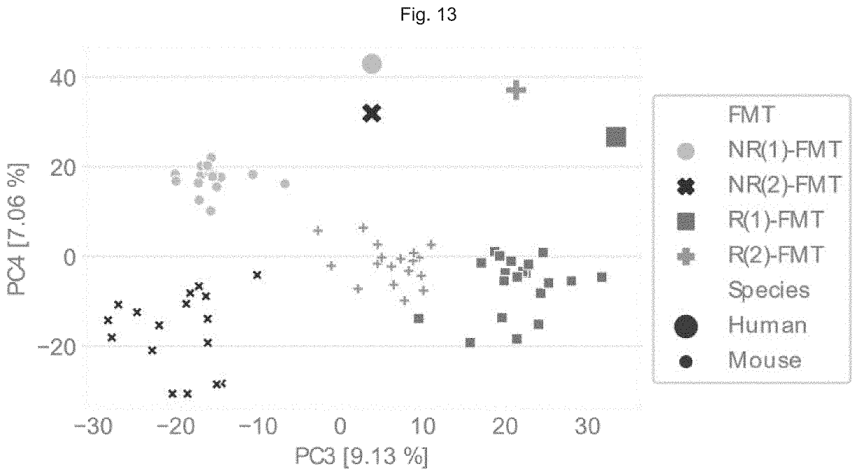

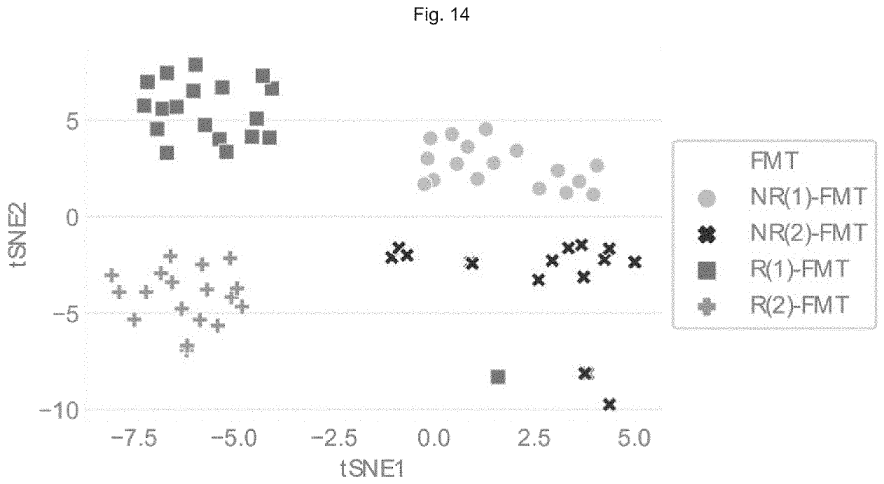

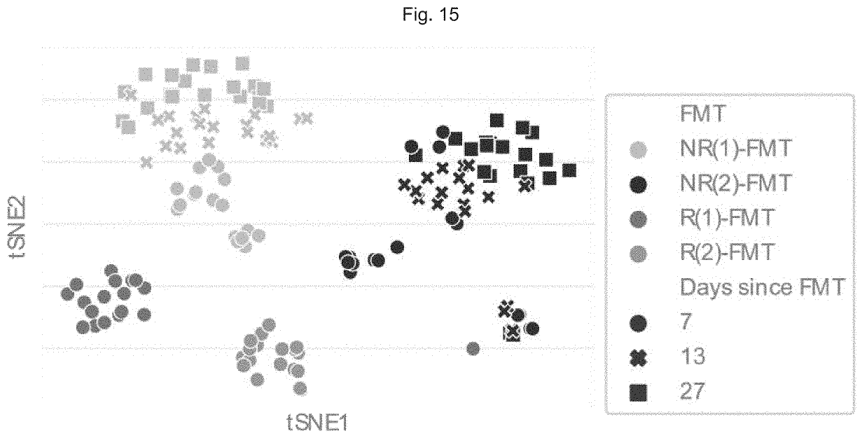

Whole genome sequencing and flow cytometry analysis were performed on human fecal and blood samples, respectively, as described in Example 9. A machine learning model was fit to discriminate cancer and control samples, using all fecal data collected to date. The model was validated using leave-one-out cross-validation, and performance evaluated using a receiver operating characteristic curve (FIG. 8 and Table 4). Organisms were then scored based on a composite score accounting for their correlations to immune markers (FIG. 9 and Tables 5 and 6) and fold change between cancer and control cohorts. The organisms were also ranked according to differential abundance between responder and non-responder patients (FIG. 10 and Table 7). As a healthy immune response characterized by an increase in CD8+ and NK-like T cells is important for rapid viral clearance (Apt et al. (2012), Immunity 37:158-170; Thevarajan et al. (2020), Nature Med. https://doi.org/10.1038/s41591-020-0819-2), microbes identified this way are predicted to be beneficial for recovery from viral infections such as influenza or COVID-19. Indeed, similar immune marker correlations are seen with the data from the cancer patient study (Table 5). Classification of responders and non-responders also implies robust and insufficient immune response, respectively, and thus serves as a proxy for response to viral infections.

TABLE 4
A random forest classifier was trained to classify operational species unit abundances for
a sample as corresponding to cancer or control. A ROC curve was generated on 145 cancer
samples and 88 control samples using leave-one-out cross validation. Following validation,
the model was trained on all the samples and feature importance values are reported.

| Feature Importance | log 10 of Fold Change | Organism Name |
|---|---|---|
| (Random Forest) | (Control vs Cancer) | (Operational Species Unit) |
| 0.016501858 | −0.415683892 | Blautia sp. AF19-10LB C2906 |
| 0.013518985 | −0.764382216 | Erysipelotrichaceae bacterium GAM147 C2844 |
| 0.010943304 | 0.280259145 | Flavonifractor plautii C2284 |
| 0.009899023 | −0.565340143 | Firmicutes bacterium AF12-30 C2644 |
| 0.009291084 | −0.557690435 | Ruminococcus sp. OF03-6AA C2904 |
| 0.008763332 | −0.52436559 | Coprobacillus sp. 8_1_38FAA C2606 |
| 0.008543128 | −0.370730577 | Eubacterium ramulus C2852 |
| 0.008185491 | 0.314786908 | [Clostridium] symbiosum C2238 |
| 0.00777239 | −0.449283525 | Coprococcus comes C2152 |
| 0.007387547 | −0.432405099 | Dorea sp. AM58-8 C2913 |
| 0.007370147 | 0.386220508 | Streptococcus vestibularis C7338 |
| 0.00712668 | −0.436129729 | Dorea longicatena C2413 |
| 0.006857525 | 0.266781049 | Catenibacterium sp. AM22-15 C2888 |
| 0.00606504 | 0.249321416 | [Clostridium] bolteae C2137 |
| 0.006038427 | 0.629434999 | [Clostridium] scindens C2143 |
| 0.005741584 | 0.559795019 | Blautia sp. N6H1-15 C2865 |
| 0.005164589 | −0.516206872 | Dorea longicatena C2131 |
| 0.005038218 | 0.440453628 | Clostridiales bacterium TF09-2AC C2150 |
| 0.004962784 | 0.089210304 | Parabacteroides merdae C0130 |
| 0.00488605 | −0.442770588 | Dorea sp. OM07-5 C2890 |
| 0.00482885 | −0.353929055 | Anaerostipes hadrus C2144 |
| 0.004801012 | 0.531527103 | Blautia hansenii C3044 |
| 0.004709165 | 0.446480902 | Anaerostipes caccae C2134 |
| 0.004700494 | 0.204253574 | Alistipes senegalensis C0284 |
| 0.004668466 | 0.189644361 | Hungatella hathewayi C2175 |
| 0.004549795 | 0.246863312 | Alistipes sp. An66 C0846 |
| 0.004488856 | −0.201826507 | Fusicatenibacter saccharivorans C2643 |
| 0.004384083 | −0.504914016 | Blautia obeum C2129 |
| 0.004369678 | 0.341085636 | Lactobacillus fermentum C3433 |
| 0.004361877 | 0.198581902 | Oscillibacter sp. PEA192 C2443 |
| 0.00430547 | −0.486272557 | Phascolarctobacterium succinatutens YIT 12067 C2237 |
| 0.00427777 | −0.490502377 | Bifidobacterium catenulatum C0014 |
| 0.004249632 | 0.185525714 | Angelakisella massiliensis C3120 |
| 0.004222488 | −0.352856347 | Ruminococcus callidus C2440 |
| 0.004185622 | 0.324033352 | Bifidobacterium dentium C0003 |
| 0.004155963 | 0.253779907 | Extibacter muris C2915 |
| 0.004044015 | 0.507281373 | [Clostridium] clostridioforme AGR2157 C2412 |
| 0.004017688 | 0.47474479 | [Clostridium] lavalense C2843 |
| 0.004004597 | −0.163492912 | Clostridium sp. AM18-55 C2845 |
| 0.003953855 | 0.181627961 | Clostridia bacterium UC5.1-1D1 C2633 |
| 0.003917395 | 0.231236234 | Streptococcus parasanguinis C4037 |
| 0.003901166 | 0.40868596 | Streptococcus mutans C3345 |
| 0.003875451 | −0.421426117 | Anaerobutyricum hallii C2206 |
| 0.003867169 | 0.259650758 | Erysipelatoclostridium ramosum C2142 |
| 0.003761301 | 0.375605827 | Paraprevotella clara C0224 |
| 0.003659752 | 0.400215198 | Eubacteriaceae bacterium CHKCI004 C2759 |
| 0.003549486 | −0.727951095 | Collinsella sp. AM34-10 C1986 |
| 0.003509696 | 0.195100824 | Flavonifractor sp. An9 C2755 |
| 0.003494686 | −0.348031554 | Ruminococcus sp. AF46-10NS C2926 |
| 0.003477621 | −0.206071101 | Clostridium sp. OM02-18AC C2931 |
| 0.003447056 | 0.457446985 | Dorea sp. Marseille-P4003 C3269 |
| 0.003386838 | 0.440006771 | Blautia producta C2356 |
| 0.00337533 | −0.305064647 | Firmicutes bacterium TM09-10 C2909 |
| 0.003362471 | 0.273578745 | Phocea massiliensis C2631 |
| 0.003322609 | 0.009697135 | Merdibacter massiliensis C3221 |
| 0.003256256 | −0.247491324 | Oscillibacter sp. ER4 C2580 |
| 0.003236909 | 0.547982486 | Clostridiales bacterium VE202-09 C2460 |
| 0.003178005 | 0.309883036 | Harryflintia acetispora C2880 |
| 0.003172428 | 0.224781595 | Flavonifractor sp. An82 C2757 |
| 0.00315518 | 0.405385756 | Streptococcus sp. HSISS2 C4629 |
| 0.003153913 | 0.185518308 | Eisenbergiella massiliensis C2435 |
| 0.003099824 | 0.309145503 | Clostridium sp. SN20 C3256 |
| 0.003032509 | 0.190748088 | Butyricicoccus porcorum C2752 |
| 0.002974263 | 0.21217243 | Bifidobacterium scardovii C0042 |
| 0.002943309 | −0.183114283 | Firmicutes bacterium AM10-47 C2889 |
| 0.002906076 | −0.344627962 | Blautia sp. TF11-31AT C2841 |
| 0.0029012 | 0.069794378 | Bacteroides clarus C0195 |
| 0.0028804 | 0.218206762 | Lachnoclostridium sp. An14 C2775 |
| 0.00287576 | −0.103576385 | Bacteroides uniformis C0132 |
| 0.002848749 | −0.167126002 | Firmicutes bacterium AF36-3BH C2905 |
| 0.002824076 | 0.309520673 | Clostridiales bacterium CCNA10 C2953 |
| 0.002809724 | 0.314889985 | Dorea sp. 5-2 C2378 |
| 0.002808948 | 0.251398969 | Clostridium sp. AT4 C2666 |
| 0.002808102 | −0.399757353 | Christensenella minuta C2682 |
| 0.002796624 | 0.40212407 | Acidaminococcus intestini C2208 |
| 0.00277314 | −0.349179114 | Massilioclostridium coli C3076 |
| 0.002759323 | 0.447842365 | Streptococcus gordonii C3645 |
| 0.002718198 | −0.312636412 | Ruminococcus sp. AF14-10 C2897 |
| 0.00270911 | −0.304912298 | Odoribacter sp. AF21-41 C0847 |
| 0.002652152 | 0.136177714 | Anaeromassilibacillus sp. An200 C2765 |
| 0.002620021 | 0.243049812 | Blautia hansenii C2161 |
| 0.002615116 | 0.233225815 | Lachnoclostridium sp. An298 C2760 |
| 0.002611426 | −0.032159744 | Roseburia faecis C2648 |
| 0.002606658 | −0.304525941 | [Ruminococcus] torques C2636 |
| 0.002587424 | 0.352092737 | Dialister pneumosintes C2708 |
| 0.002576223 | 0.155095629 | Bacteroides caccae C0156 |
| 0.002573354 | 0.192538373 | Butyricimonas sp. Marseille-P4593 C1362 |
| 0.002552093 | −0.244156505 | Clostridiales bacterium VE202-01 C2458 |
| 0.002548102 | 0.361035514 | Blautia sp. An249 C2761 |
| 0.002517171 | −0.394453097 | Turicibacter sanguinis C2220 |
| 0.002506636 | 0.277784722 | Enorma massiliensis C1943 |
| 0.002501752 | 0.275045721 | Streptococcus sp. HSISM1 C4627 |
| 0.00249105 | −0.534927741 | Raoultibacter massiliensis C2013 |
| 0.002478591 | 0.165198022 | Ruminococcaceae bacterium AM07-15 C2928 |
| 0.002471317 | −0.392581823 | Clostridium sp. AF36-4 C2893 |
| 0.002468113 | 0.175364006 | Eubacterium sp. 3_1_31 C2186 |
| 0.002461251 | −0.215314373 | Clostridiales bacterium AM23-16LB C2886 |
| 0.002456084 | 0.016078272 | Tyzzerella nexilis C2155 |
| 0.002443061 | 0.267120144 | Sellimonas intestinalis C2461 |
| 0.002440381 | −0.295779942 | Butyricicoccus sp. AM29-23AC C2943 |
| 0.002429933 | −0.160088535 | Alistipes putredinis DSM 17216 C0133 |
| 0.002403414 | −0.34579043 | Firmicutes bacterium AF25-13AC C2695 |
| 0.002389793 | 0.233921669 | [Clostridium] citroniae C2272 |
| 0.002388663 | −0.287905997 | Faecalibacterium prausnitzii C2809 |
| 0.002377287 | 0.265105676 | Collinsella intestinalis C1929 |
| 0.002371557 | 0.325006666 | Lachnoclostridium sp. An196 C2766 |
| 0.002331412 | 0.161011335 | Ruthenibacterium lactatiformans C2282 |
| 0.00232152 | −0.257009611 | Ruminococcus sp. AF21-42 C2938 |
| 0.002321468 | −0.069789147 | Butyrivibrio crossotus DSM 2876 C2154 |
| 0.002319772 | 0.003128369 | Bacteroides vulgatus C0099 |
| 0.00229641 | 0.091890638 | Bacteroides acidifaciens C0604 |
| 0.002277453 | 0.195676439 | Flavonifractor sp. An10 C2786 |
| 0.002276704 | −0.046379748 | Drancourtella sp. An177 C2763 |
| 0.002272041 | 0.160264471 | Anaerotruncus colihominis C2145 |
| 0.00225912 | −0.120363782 | Pseudoflavonifractor capillosus ATCC 29799 C2198 |
| 0.002256141 | −0.517722756 | Bifidobacterium bifidum C0005 |
| 0.002250462 | −0.094755491 | Anaeromassilibacillus sp. Marseille-P3876 C2925 |
| 0.002249251 | 0.292298556 | Coprobacter fastidiosus C0231 |
| 0.002245262 | 0.338335356 | Bariatricus massiliensis C3067 |
| 0.002237507 | 0.162776529 | Coprococcus sp. AF21-14LB C2900 |
| 0.002226962 | −0.408971832 | Clostridiaceae bacterium OM08-6BH C2949 |
| 0.002218309 | −0.002771039 | [Bacteroides] pectinophilus ATCC 43243 C2151 |
| 0.002217958 | 0.25729072 | Pseudoflavonifractor sp. An184 C2770 |
| 0.002200796 | −0.200989635 | Eubacterium sp. AM18-26 C2923 |
| 0.00218927 | −0.081898577 | Parabacteroides sp. AF18-52 C1227 |
| 0.002187486 | −0.123209619 | Coprococcus eutactus C2642 |
| 0.002161484 | 0.30195464 | Phascolarctobacterium faecium C2862 |
| 0.002158572 | −0.087502084 | Lachnospiraceae bacterium OM04-12BH C2952 |
| 0.002148262 | 0.047074352 | Parabacteroides distasonis C0100 |
| 0.002142893 | −0.255666512 | Faecalibacterium sp. AF28-13AC C2810 |
| 0.002134925 | −0.158568078 | Bacteroides stercoris C0134 |
| 0.002114346 | −0.355662173 | Firmicutes bacterium AM41-11 C2946 |
| 0.002110017 | −0.165551333 | [Clostridium] amygdalinum C2887 |
| 0.00210878 | 0.250557414 | Anaerotignum lactatifermentans C2790 |
| 0.002107104 | 0.436062031 | [Clostridium] aldenense C2884 |
| 0.002095506 | 0.084877313 | Intestinimonas timonensis C3301 |
| 0.002094298 | 0.256448208 | Alistipes finegoldii C0177 |
| 0.002084535 | 0.058630203 | Mordavella sp. Marseille-P3756 C3280 |
| 0.002082758 | 0.128186268 | Streptococcus oralis subsp. tigurinus C6034 |
| 0.002078318 | 0.085608213 | Prevotella sp. P3-92 C0874 |
| 0.002078069 | 0.213972847 | Alterileibacterium massiliense C3118 |
| 0.002056041 | −0.041470873 | Coprococcus eutactus C2140 |
| 0.00205311 | 0.306957134 | Fusobacterium nucleatum C2028 |
| 0.002052465 | −0.248224092 | Massilimaliae massiliensis C3228 |
| 0.00204697 | −0.360378609 | Clostridium sp. AM33-3 C2947 |
| 0.00204635 | −0.169578698 | Firmicutes bacterium AM29-6AC C2940 |
| 0.002030614 | 0.14595452 | Hungatella hathewayi C2351 |
| 0.00202297 | −0.251539605 | Blautia luti C2436 |
| 0.001993254 | −0.189503492 | Holdemanella biformis C2160 |
| 0.001989672 | −0.240687636 | Anaerobutyricum hallii C3263 |
| 0.001971269 | 0.089118345 | Alistipes shahii C0199 |
| 0.001965797 | 0.274298289 | Odoribacter laneus YIT 12061 C0239 |
| 0.001965483 | 0.078208985 | Peptoniphilus lacrimalis C2213 |
| 0.00194357 | 0.120085576 | Streptococcus constellatus C4635 |
| 0.001936923 | −0.130171095 | Eubacterium sp. AF15-50 C2941 |
| 0.001934746 | 0.058661303 | Clostridiales bacterium CHKCI006 C3057 |
| 0.00193182 | 0.157031233 | Alistipes onderdonkii C0322 |
| 0.001930949 | 0.599613718 | Lactobacillus salivarius C3392 |
| 0.001892559 | 0.121653741 | Neglecta timonensis C3059 |
| 0.001887608 | 0.232534892 | Clostridium sp. 1001271st1 H5 C3046 |
| 0.001866112 | 0.045627845 | Prevotellamassilia timonensis C1705 |
| 0.001865236 | 0.16241841 | Slackia exigua C1932 |
| 0.001854461 | −0.219463054 | Bacteroides finegoldii C0138 |
| 0.001852121 | −0.224535064 | Barnesiella intestinihominis C0275 |
| 0.001841628 | −0.224153342 | Eubacterium ventriosum C2128 |
| 0.001839575 | 0.14243286 | Streptococcus anginosus C4636 |
| 0.001839422 | 0.049603186 | Prevotella sp. BCRC 81118 C1221 |
| 0.001838081 | 0.080289666 | Akkermansia sp. aa_0143 C1922 |
| 0.001836116 | 0.387030309 | Blautia sp. Marseille-P3201T C3179 |
| 0.001832526 | −0.319221468 | Ruminococcus lactaris C2149 |
| 0.001830134 | −0.187587381 | Eubacterium sp. AF34-35BH C2902 |
| 0.001829468 | 0.227277401 | Paraprevotella xylaniphila C0198 |
| 0.001821326 | −0.005377338 | Alistipes sp. 5CPEGH6 C1580 |
| 0.001819158 | −0.045467144 | Eubacterium sp. TM06-47 C2917 |
| 0.001812327 | −0.36246911 | Faecalibacterium prausnitzii C2651 |
| 0.001807702 | 0.407496562 | Lachnoclostridium sp. An118 C2782 |
| 0.001804296 | 0.162945863 | Bacteroides sp. AM10-21B C1214 |
| 0.001801377 | −0.612948492 | Collinsella aerofaciens C1977 |
| 0.001799781 | 0.200783717 | Ruminococcaceae bacterium D16 C2214 |
| 0.001795815 | −0.230812001 | Dorea formicigenerans C2197 |
| 0.001782118 | 0.050805612 | [Clostridium] leptum C2136 |
| 0.001769616 | 0.255735482 | Parabacteroides johnsonii C0139 |
| 0.001757969 | 0.335044837 | [Clostridium] methylpentosum DSM 5476 C2167 |
| 0.001748845 | 0.137798554 | Parabacteroides sp. SN4 C1840 |
| 0.001732845 | −0.088799912 | Clostridium sp. YH-panp20 C2971 |
| 0.001730465 | 0.187542345 | [Ruminococcus] gnavus C2199 |
| 0.001721291 | 0.245585432 | Holdemania sp. Marseille-P2844 C3176 |
| 0.001711469 | 0.326189698 | [Clostridium] asparagiforme C2165 |
| 0.001709265 | −0.32340108 | Ruminococcus sp. AM42-11 C2945 |
| 0.001708751 | −0.211956107 | Blautia sp. OF03-15BH C2912 |
| 0.001705071 | −0.326716726 | Subdoligranulum sp. APC924/74 C2870 |
| 0.001704797 | −0.391815999 | Romboutsia timonensis C3123 |
| 0.001697621 | 0.114228311 | Streptococcus oralis C5466 |
| 0.0016965 | −0.048932599 | Clostridium sp. AF34-13 C2653 |
| 0.001691772 | 0.20344874 | Dialister invisus DSM 15470 C2174 |
| 0.001689134 | 0.095511852 | Olsenella uli C1928 |
| 0.001673536 | −0.100055999 | [Eubacterium] siraeum C2135 |
| 0.001662325 | 0.122632002 | Akkermansia muciniphila C1917 |
| 0.001656155 | 0.214252057 | Faecalimonas umbilicata C2244 |
| 0.001642083 | 0.182409993 | Clostridiales bacterium Marseille-P5551 C3291 |
| 0.001637493 | 0.004280452 | Ruminococcaceae bacterium C2861 |
| 0.001634056 | 0.134322164 | Lactonifactor longoviformis C2830 |
| 0.00162656 | 0.485421565 | Lactobacillus rhamnosus C3457 |
| 0.001625673 | 0.273598423 | Coriobacteriaceae bacterium CHKCI002 C1973 |
| 0.001624111 | −0.011206269 | Anaerofilum sp. An201 C2764 |
| 0.001623073 | 0.072560155 | Bacteroides stercorirosoris C0463 |
| 0.001622251 | −0.202159024 | Alistipes sp. CHKCI003 C1653 |
| 0.001620599 | 0.174483602 | Anaeromassilibacillus sp. Marseille-P3371 C2632 |
| 0.001619706 | 0.30918881 | Bacteroides sp. HF-5092 C1596 |
| 0.001619395 | 0.147450868 | Bacteroides coprocola C0136 |
| 0.001617633 | −0.087543931 | Blautia obeum C2901 |
| 0.001614518 | 0.34599988 | Evtepia gabavorous C2876 |
| 0.001613136 | −0.161787704 | Ruminococcus sp. AF31-8BH C2903 |
| 0.001603563 | 0.123138967 | Anaerococcus sp. HMSC068A02 C2185 |
| 0.001598053 | 0.202218832 | Lactobacillus plantarum C3798 |
| 0.001594311 | −0.488328224 | Allisonella histaminiformans C3105 |
| 0.001586576 | −0.098634411 | Roseburia intestinalis C2158 |
| 0.001584302 | −0.452532206 | Bifidobacterium pseudocatenulatum C0013 |
| 0.001572828 | −0.061735341 | Alistipes sp. 5CBH24 C0283 |
| 0.001570429 | 0.116445939 | Streptococcus salivarius C4352 |
| 0.001563761 | −0.1456938 | Gordonibacter pamelaeae C1937 |
| 0.001552982 | −0.476696467 | Collinsella aerofaciens C1933 |
| 0.001550017 | 0.146736525 | Flavonifractor sp. An92 C2753 |
| 0.001546685 | −0.312675608 | Clostridium sp. OF10-22XD C2132 |
| 0.001544022 | 0.143206979 | Haemophilus parainfiuenzae T3T1 C4194 |
| 0.001541656 | 0.177526741 | Streptococcus gallolyticus C3902 |
| 0.001538447 | −0.306056064 | Bacteroides heparinolyticus C1005 |
| 0.00153663 | −0.11819143 | Eubacterium sp. OM08-24 C2896 |
| 0.001535096 | −0.242001524 | Faecalibacterium prausnitzii C2863 |
| 0.001532366 | −0.074691634 | Bacteroides nordii C0263 |
| 0.00153067 | −0.077986369 | Marvinbryantia formalexigens C2205 |
| 0.00152307 | 0.128955058 | Lachnospiraceae bacterium 1_4_56FAA C2258 |
| 0.001515629 | 0.100560583 | Roseburia sp. OF03-24 C2911 |
| 0.001515375 | −0.070429456 | Lachnospiraceae bacterium AM48-27BH C2935 |
| 0.00151401 | 0.209874419 | Fusobacterium nucleatum C2027 |
| 0.001504779 | −0.142905059 | Clostridium sp. OF09-36 C2944 |
| 0.001497974 | 0.032287603 | Peptostreptococcus anaerobius C2217 |
| 0.00149702 | −0.090219746 | Leuconostoc mesenteroides C3570 |
| 0.001495408 | 0.419154573 | Blautia producta C2581 |
| 0.001489385 | 0.10891937 | Bacteroides cellulosilylicus C0143 |
| 0.001487732 | −0.474788291 | Faecalibacterium prausnitzii C2184 |
| 0.00147645 | 0.378916316 | Lachnoclostridium sp. An181 C2771 |
| 0.001467508 | −0.26283664 | Clostridium sp. AM49-4BH C2934 |
| 0.001467098 | 0.000605824 | Clostridium sp. ATCC 29733 C2438 |
| 0.0014621 | −0.322415354 | Blautia sp. KGMB01111 C3003 |
| 0.001454853 | 0.095997825 | Clostridioides difficile C2074 |
| 0.001447136 | −0.061440984 | Parvimonas micra C2139 |
| 0.001444928 | 0.212594053 | Megasphaera sp. DISK 18 C2433 |
| 0.001443122 | 0.285426008 | Bacteroides salyersiae C0264 |
| 0.001438622 | −0.046750403 | Lactobacillus paracasei C3573 |
| 0.00143852 | −0.082129699 | Eggerthella timonensis C2011 |
| 0.001425959 | −0.114661776 | Bifidobacterium animalis C0002 |
| 0.001416675 | 0.280368137 | Klebsiella variicola C3709 |
| 0.001414944 | −0.246601387 | Agathobaculum butyriciproducens C2850 |
| 0.001405704 | 0.074907434 | Anaeromassilibacillus sp. An250 C2762 |
| 0.001402711 | −0.081852178 | Ruminococcus sp. AF24-32LB C2894 |
| 0.001385668 | −0.358288898 | Faecalibacterium prausnitzii C2138 |
| 0.001385102 | −0.035320328 | Streptococcus mitis NCTC 12261 C4004 |
| 0.001379637 | 0.168782631 | Prevotella sp. AM23-5 C0872 |
| 0.001378158 | 0.138984788 | Collinsella tanakaei C1938 |
| 0.001375186 | 0.128316545 | Intestinimonas butyriciproducens C2577 |
| 0.001357814 | −0.130545436 | Gemmiger formicilis C3234 |
| 0.001356921 | 0.099487524 | Culturomica massiliensis C1230 |
| 0.001349152 | −0.028077053 | Roseburia sp. AM51-8 C2924 |
| 0.001346043 | 0.172383478 | Eubacterium sp. An11 C2784 |
| 0.001345379 | 0.067714209 | Hungatella hathewayi C2462 |
| 0.001342127 | 0.190693108 | Bacteroides rodentium JCM 16496 C0461 |
| 0.001325512 | −0.073609518 | Clostridium sp. TM06-18 C2922 |
| 0.001314021 | −0.14364999 | Clostridium sp. AF27-2AA C2937 |
| 0.001303967 | −0.118957311 | Parabacteroides sp. TM07-1AC C1229 |
| 0.001301855 | 0.049387119 | Butyricimonas sp. Marseille-P2440 C0330 |
| 0.001297003 | −0.022569114 | Neobitarella massiliensis C3275 |
| 0.001291043 | −0.159405658 | Clostridium sp. AM30-24 C2942 |
| 0.001276208 | −0.060522193 | Prevotella sp. Marseille-P4119 C1902 |
| 0.001268369 | 0.116021978 | Clostridium perfringens C2078 |
| 0.001264612 | −0.0200892 | Bacteroides sp. An19 C0842 |
| 0.001263236 | 0.301353216 | Klebsiella pneumoniae C3423 |
| 0.001260612 | −0.160766973 | Alistipes timonensis C0271 |
| 0.001256742 | 0.252094618 | Salmonella enterica C3329 |
| 0.001253605 | 0.178630171 | Intestinimonas massiliensis C2614 |
| 0.001252735 | 0.470799178 | Cuneatibacter caecimuris C3008 |
| 0.001241543 | 0.105626404 | Eubacterium brachy ATCC 33089 C2452 |
| 0.001233195 | −0.111096287 | Eisenbergiella tayi C2259 |
| 0.001231803 | 0.203084745 | Akkermansia muciniphila C1923 |
| 0.001229663 | 0.07528375 | Akkermansia muciniphila C1921 |
| 0.001227806 | 0.316271739 | Metaprevotella massiliensis C1901 |
| 0.001223817 | 0.103266649 | Streptococcus intermedius C4476 |
| 0.001223003 | −0.009215998 | Desulfovibrio piger C7227 |
| 0.001210017 | −0.103823837 | Eubacterium ramulus C2442 |
| 0.001208958 | −0.066912759 | Clostridium sp. OM07-10AC C2948 |
| 0.001208297 | −0.011533879 | Faecalicatena fissicatena C2241 |
| 0.001206301 | −0.14769711 | Clostridium sp. AF23-8 C2908 |
| 0.001201907 | 0.087391156 | Klebsiella michiganensis C4315 |
| 0.001201625 | 0.090163662 | Collinsella sp. AF08-23 C1987 |
| 0.001199225 | 0.047629461 | Megasphaera cerevisiae C2604 |
| 0.00119489 | 0.157003749 | Lachnoclostridium sp. An138 C2776 |
| 0.001192374 | 0.346071847 | Eubacterium limosum C2659 |
| 0.001183998 | 0.163715553 | Streptococcus pneumoniae C3327 |
| 0.001173126 | 0.161269394 | Eubacterium callanderi C2127 |
| 0.001161929 | −0.321742198 | Ruminococcus champanellensis C2249 |
| 0.001157051 | −0.04739511 | Catenibacterium mitsuokai DSM 15897 C2204 |
| 0.001154034 | 0.069882758 | Streptococcus sanguinis C3561 |
| 0.001152159 | −0.229970619 | Firmicutes bacterium AF22-6AC C2933 |
| 0.001149193 | −0.093698754 | Roseburia sp. OM04-15AA C2892 |
| 0.001148872 | −0.136898288 | Holdemania massiliensis AP2 C2339 |
| 0.00114848 | −0.143597792 | Olsenella sp. AF21-51 C1985 |
| 0.001145605 | 0.041391195 | Bacteroides ovatus C0131 |
| 0.001144548 | 0.310613625 | Eggerthella sp. YY7918 C1941 |
| 0.001142328 | 0.294636274 | Lachnospiraceae bacterium 2_1_46FAA C2247 |
| 0.001139964 | −0.109907027 | Anaerostipes sp. 992a C2729 |
| 0.001136248 | 0.071917201 | Eggerthella lenta C1927 |
| 0.001127673 | −0.035851608 | Streptococcus sp. ChDC B345 C6537 |
| 0.00112536 | 0.235371201 | Ruminococcus sp. AF18-22 C2662 |
| 0.001124558 | 0.22935135 | Blautia sp. An81 C2788 |
| 0.001120621 | −0.502954606 | Ruminococcus sp. KGMB03662 C2557 |
| 0.001117895 | −0.016216198 | Bacteroides sp. OF04-15BH C1226 |
| 0.001117113 | −0.317608637 | Eubacterium sp. AF22-8LB C2898 |
| 0.001116776 | −0.13214399 | Candidatus Borkfalkia ceftriaxoniphila C3005 |
| 0.001115975 | −0.245958899 | Gordonibacter urolithinfaciens C1971 |
| 0.001114616 | −0.335185925 | Bifidobacterium adolescentis C0001 |
| 0.001114192 | 0.083292114 | Eubacterium pyruvativorans C3098 |
| 0.001113405 | −0.113942218 | Massilimaliae timonensis C3250 |
| 0.001111358 | −0.321124776 | Clostridium disporicum C2479 |
| 0.001108373 | 0.416260181 | Bacteroides zoogleoformans C1004 |
| 0.001099862 | 0.103183512 | Bacteroides sartorii C0346 |
| 0.001096801 | 0.127258668 | Finegoldia magna C2170 |
| 0.001096565 | 0.053902093 | Burkholderiales bacterium YL45 C5482 |
| 0.001090767 | −0.25222383 | Bacteroides mediterraneensis C1791 |
| 0.001089194 | −0.192935162 | Clostridium sp. AF46-9NS C2891 |
| 0.001085672 | −0.022510129 | Bacteroides faecis C0221 |
| 0.001084937 | 0.183744827 | Enteroscipio rubneri C1978 |
| 0.001080288 | 0.217242623 | Streptococcus agalactiae C3342 |
| 0.001077696 | 0.014956563 | Oscillibacter ruminantium GH1 C2321 |
| 0.001071923 | 0.226961129 | Bacteroides coprophilus C0141 |
| 0.001070282 | −0.085202725 | Prevotella sp. 885 C0883 |
| 0.001068757 | 0.41779361 | Blautia hominis C2806 |
| 0.00106737 | 0.227560508 | Fusobacterium nucleatum C2023 |
| 0.001063571 | −0.005996163 | Alistipes sp. Marseille-P2431 C1656 |
| 0.001046414 | −0.131247999 | Christensenella sp. Marseille-P3954 C3290 |
| 0.001046021 | 0.073482048 | Blautia hydrogenotrophica C2163 |
| 0.001034582 | 0.033741303 | Escherichia coli C6189 |
| 0.001034232 | 0.000419447 | Bacteroides plebeius C0183 |
| 0.001033161 | 0.037947008 | Eubacterium limosum C2585 |
| 0.001031559 | 0.231894983 | Bacteroides sp. NM69_E16B C1512 |
| 0.00102259 | −0.332512425 | Olsenella sp. Marseille-P4518 C1983 |
| 0.001019694 | −0.164199636 | Lachnoanaerobaculum saburreum C2233 |
| 0.001017125 | −0.206044424 | Clostridium sp. AF20-17LB C2921 |
| 0.001013385 | −0.159145062 | Bifidobacterium angulatum C0006 |
| 0.001011242 | −0.124685694 | Coprococcus sp. OM04-5BH C2951 |
| 0.001010502 | 0.199924075 | Bacteroides caecimuris C0768 |
| 0.001005476 | −0.054514013 | Paramuribaculum intestinale C1027 |
| 0.001002001 | 0.065282268 | Bacteroides eggerthii C0137 |
| 0.001001469 | −0.069431173 | Pseudoflavonifractor sp. An44 C2769 |
| 0.00100062 | 0.224179803 | Bacteroides togonis C1815 |
| 0.000998879 | −0.079349954 | Enterorhabdus caecimuris C1946 |
| 0.000996811 | −0.035659589 | Butyricicoccus pullicaecorum C2367 |
| 0.000996394 | 0.119454752 | Lachnospiraceae bacterium KGMB03038 C3054 |
| 0.000988689 | −0.095646493 | Clostridium sp. SY8519 C2300 |
| 0.00098773 | −0.244190108 | Bifidobacterium ruminantium C0033 |
| 0.000983787 | 0.167974308 | Veillonella dispar C2172 |
| 0.000981089 | 0.009434997 | Faecalibacterium sp. An122 C2768 |
| 0.000971714 | −0.078320362 | Paraeggerthella hongkongensis C1991 |
| 0.000970657 | −0.061838315 | Bacteroides faecichinchillae C0462 |
| 0.000970589 | −0.100958093 | Veillonella seminalis C2333 |
| 0.000966201 | −0.203389419 | Anaerofustis stercorihominis C3043 |
| 0.000965329 | −0.127606155 | Gabonia massiliensis C0573 |
| 0.000958921 | 0.097531327 | Lachnospiraceae bacterium C7401 |
| 0.000955835 | 0.220644706 | Clostridia bacterium UC5.1-1D10 C2630 |
| 0.000946293 | −0.119032203 | Parabacteroides acidifaciens C1178 |
| 0.000939111 | −0.491867958 | Collinsella sp. TM05-38 C1984 |
| 0.000937568 | 0.238492033 | Veillonella parvula C2108 |
| 0.000932801 | 0.088210302 | Gemmiger sp. An50 C2791 |
| 0.000932461 | 0.080276705 | Bacteroides pyogenes C0391 |
| 0.000932048 | 0.20638792 | Lachnoclostridium sp. An76 C2789 |
| 0.000931273 | −0.417870861 | Faecalibacterium prausnitzii C2650 |
| 0.00093091 | 0.034378724 | Drancourtella sp. An57 C2780 |
| 0.000930578 | −0.057174001 | Desulfovibrio sp. G11 C3781 |
| 0.000927044 | 0.214918452 | Faecalicatena orotica C2855 |
| 0.000926301 | 0.080750766 | [Ruminococcus] torques C2130 |
| 0.000924352 | −0.052296196 | Coprobacillus cateniformis C2235 |
| 0.000924235 | −0.30548312 | Prevotella stercorea C0227 |
| 0.000922776 | 0.214718723 | Enterobacter asburiae C4744 |
| 0.000921102 | 0.275685331 | Streptococcus lutetiensis C4617 |
| 0.000908652 | −0.209498347 | Bacteroides massiliensis C0310 |
| 0.000902209 | 0.024387818 | Anaerofustis stercorihominis C2147 |
| 0.000897276 | −0.417096051 | Senegalimassilia anaerobia C1940 |
| 0.000895988 | 0.122269666 | Clostridium cadaveris C2409 |
| 0.000894405 | −0.129710456 | Eubacterium coprostanoligenes C3232 |
| 0.000892552 | 0.092455818 | Streptococcus infantarius subsp. infantarius CJ18 C4334 |
| 0.000889081 | −0.157473973 | Clostridiales bacteriumMarseille-P2846 C3254 |
| 0.000885777 | 0.084144866 | Lachnoclostridium sp. An169 C2774 |
| 0.000885709 | −0.011837149 | Bacteroides fragilis C0096 |
| 0.000885092 | −0.096838499 | Intestinibacter bartlettii C2141 |
| 0.000884226 | 0.102943242 | Absiella dolichum C2133 |
| 0.000879993 | 0.276721768 | Bacteroides intestinalis C1222 |
| 0.000874022 | −0.176033978 | Lachnospiraceae bacterium OF09-6 C2885 |
| 0.000871852 | 0.11799681 | Lachnoclostridium edouardi C3267 |
| 0.000867157 | 0.03000888 | Bacteroides timonensis C0434 |
| 0.000859738 | −0.191448288 | [Clostridium] spiroforme C2146 |
| 0.000854106 | 0.032964866 | Streptococcus sp. I-G2 C4650 |
| 0.000852642 | 0.193752289 | [Clostridium] clostridioforme C2275 |
| 0.000850375 | −0.107533876 | Alistipes ihumii AP11 C0292 |
| 0.00084566 | 0.029668283 | [Clostridium] innocuum C2230 |
| 0.000841331 | −0.182746209 | Leuconostoc lactis C5492 |
| 0.000837107 | −0.148687377 | Lactococcus lactis C3409 |
| 0.000833791 | 0.075233896 | Bifidobacterium gallinarum C0040 |
| 0.000832892 | −0.052348168 | Lachnospira pectinoschiza C2649 |
| 0.000819471 | 0.044124824 | Clostridium tertium C2166 |
| 0.000818078 | 0.013262705 | Bacteroides gallinarum C0320 |
| 0.000816004 | −0.007624252 | Gardnerella vaginalis C0077 |
| 0.000814064 | 0.124276378 | Candidatus Stoquefichus sp. KLE1796 C2685 |
| 0.000810143 | −0.077567242 | Megamonas funiformis C2294 |
| 0.000806911 | −0.216211462 | Eubacterium sp. TM05-53 C2895 |
| 0.000805937 | −0.10501558 | Roseburia hominis C2266 |
| 0.00080548 | 0.160289033 | Actinomyces naeslundii C5308 |
| 0.00080031 | −0.040410654 | Clostridium sp. M62/1 C2168 |
| 0.000794225 | 0.016679858 | Lachnospiraceae bacterium OF09-33XD C2950 |
| 0.000784244 | 0.025757522 | Mediterranea massiliensis C1792 |
| 0.000783028 | −0.473565196 | Collinsella bouchesdurhonensis C1956 |
| 0.000780365 | 0.18073776 | Parabacteroides distasonis C1282 |
| 0.000776777 | −0.066719417 | Alistipes sp. cv1 C1225 |
| 0.000775056 | 0.215608385 | Lactobacillus paragasseri C5843 |
| 0.000774821 | 0.106290282 | Enterococcus faecalis C3356 |
| 0.000770822 | 0.044316403 | Emergencia timonensis C2919 |
| 0.000770705 | 0.007621492 | Muribaculum sp. An287 C0841 |
| 0.000765772 | −0.039039051 | Candidatus Stoquefichus sp. SB1 C2613 |
| 0.000764151 | 0.149737605 | Haemophilus parainfluenzae C6724 |
| 0.000758758 | −0.139580633 | Acidaminococcus fermentans C2110 |
| 0.000758604 | 0.014886565 | Streptococcus sp. A12 C5358 |
| 0.000757928 | 0.103430739 | Ruminococcus sp. JE7A12 C3041 |
| 0.000757477 | 0.124922464 | Anaeroglobus geminatus F0357 C2283 |
| 0.000752717 | 0.201105928 | Bacteroides sp. An322 C0849 |
| 0.000750886 | 0.092991853 | Klebsiella aerogenes C4223 |
| 0.00074905 | −0.151453151 | Firmicutes bacterium AM43-11BH C2910 |
| 0.00074725 | 0.319641701 | Citrobacter freundii C4862 |
| 0.000746863 | 0.019294667 | Lachnospiraceae bacterium C2825 |
| 0.000744408 | 0.024367545 | Collinsella stercoris DSM 13279 C1930 |
| 0.000742398 | −0.069413745 | Alistipes inops C0554 |
| 0.000740749 | 0.074724867 | Staphylococcus aureus C3394 |
| 0.000737647 | 0.166740913 | Pseudoflavonifractor sp. AF19-9AC C2939 |
| 0.000734243 | 0.047494987 | Bifidobacterium breve C0007 |
| 0.000733278 | −0.106066732 | Asaccharobacter celatus C1952 |
| 0.000733193 | 0.200658318 | Bacteroides thetaiotaomicron C0098 |
| 0.000732128 | 0.006225001 | Streptococcus mitis C5142 |
| 0.000731863 | −0.123724955 | Lactobacillus acidophilus C3484 |
| 0.000727884 | −0.197342794 | Subdoligranulum variabile DSM 15176 C2162 |
| 0.000725883 | −0.32980633 | Turicibacter sanguinis C2647 |
| 0.000724945 | 0.024395901 | Lactobacillus curvatus C5454 |
| 0.000721941 | −0.116696596 | Roseburia inulinivorans C2207 |
| 0.000719454 | 0.14576632 | Agathobaculum desmolans ATCC 43058 C2531 |
| 0.000719137 | 0.061521208 | Eisenbergiella sp. OF01-20 C2932 |
| 0.000717609 | −0.008006904 | Lawsonibacter asaccharolyticus C2612 |
| 0.000716353 | −0.27637531 | Coprococcus catus C2881 |
| 0.000714658 | −0.235792289 | Faecalibacterium prausnitzii C2864 |
| 0.000713496 | 0.044440911 | Bacteroides fluxus YIT 12057 C0196 |
| 0.000709063 | 0.057843542 | Ruminococcaceae bacterium Marseille-P2935 C3117 |
| 0.000708861 | 0.132034289 | Lactobacillus casei C4934 |
| 0.000706391 | −0.223572419 | Faecalibacterium prausnitzii C2191 |
| 0.00070492 | 0.178244024 | Escherichia coli C3313 |
| 0.000702873 | −0.053059381 | Prevotella lascolaii C1655 |
| 0.000699434 | −0.068127523 | Christensenella timonensis C3068 |
| 0.000695606 | −0.191454148 | Streptococcus thermophilus C3480 |
| 0.00068995 | −0.007031037 | Dielma fastidiosa C2331 |
| 0.000689289 | 0.054494897 | Faecalitalea sp. Marseille-P3755 C3257 |
| 0.000689111 | −0.231345103 | Dialister succinatiphilus YIT 11850 C2287 |
| 0.000687764 | −0.101689367 | Chitinophaga sp. K20C18050901 C1205 |
| 0.000683105 | −0.18109626 | Bifidobacterium longum C0000 |
| 0.000681336 | 0.121849135 | Streptococcus australis C7313 |
| 0.000680574 | −0.255797065 | Clostridium cuniculi C3022 |
| 0.000675816 | −0.101093017 | Clostridiales bacterium KA00274 C2670 |
| 0.0006733 | 0.066007328 | Erysipelatoclostridium sp. An173 C2772 |
| 0.000667452 | 0.055143325 | Pseudoflavonifractor sp. Marseille-P3106 C3237 |
| 0.000666343 | 0.27979269 | Lachnoclostridium sp. An131 C2777 |
| 0.000663042 | −0.127290782 | Ruminococcus sp. AF41-9 C2929 |
| 0.000659973 | 0.094285428 | Shuttleworthia sp. MSX8B C2176 |
| 0.00065507 | 0.110634228 | Methanobrevibacter smithii C3636 |
| 0.000649624 | −0.078446486 | Butyricimonas faecihominis C1324 |
| 0.000647276 | 0.05887023 | Massilimicrobiota timonensis C2778 |
| 0.000646901 | 0.137638451 | Bacteroides barnesiae C0323 |
| 0.0006433 | −0.134246508 | Victivallales bacterium CCUG 44730 C6246 |
| 0.000640184 | 0.122380223 | Haemophilus parainfluenzae C6455 |
| 0.000636883 | 0.064289399 | Akkermansia muciniphila C1920 |
| 0.000632492 | −0.308227618 | Catabacter hongkongensis C2600 |
| 0.000630493 | −0.363573867 | Bacteroides bouchesdurhonensis C1842 |
| 0.000622319 | −0.014535125 | Prevotella sp. P3-122 C0877 |
| 0.000619871 | 0.0165477 | Roseburia sp. 831b C2726 |
| 0.000615916 | −0.163514198 | Sutterella megalosphaeroides C6522 |
| 0.000614283 | −0.082835345 | Erysipelotrichaceae bacterium 3_1_53 C2188 |
| 0.000614281 | 0.013021903 | Holdemania filiformis C2164 |
| 0.000613954 | 0.059841271 | Alistipes sp. Marseille-P5997 C0839 |
| 0.00060878 | 0.148711311 | Blautia coccoides C2701 |
| 0.000597168 | −0.02417881 | Clostridium sp. BSD2780061688st1 E8 C3045 |
| 0.000594817 | −0.17197938 | Mogibacterium diversum C2838 |
| 0.000591669 | 0.038151561 | Fusobacterium ulcerans C2030 |
| 0.000588198 | 0.24254803 | Enterobacter cloacae C3869 |
| 0.000587106 | 0.027112536 | Monoglobus pectinilyticus C2823 |
| 0.000581387 | 0.090800994 | Prevotella oris C0118 |
| 0.000576756 | 0.144277974 | Veillonella tobetsuensis C2607 |
| 0.000574411 | −0.155129298 | Kandleria vitulina C2503 |
| 0.00057406 | 0.021398815 | Negativibacillus massiliensis C3220 |
| 0.0005648 | −0.270611264 | [Eubacterium] eligens C2123 |
| 0.000561479 | −0.00147982 | Fournierella massiliensis C2661 |
| 0.000557105 | 0.017814008 | Agathobacter ruminis C2528 |
| 0.000554427 | 0.126947262 | Acetitomaculum ruminis DSM 5522 C3147 |
| 0.000551557 | −0.119643009 | Parolsenella catena C1992 |
| 0.000546323 | 0.093101738 | Alistipes sp. An31A C0840 |
| 0.000544823 | 0.100984449 | Slackia piriformis YIT 12062 C1942 |
| 0.000542329 | 0.084136379 | Pseudoflavonifractor sp. An85 C2787 |
| 0.000541822 | 0.150927143 | Enterococcus faecium C4060 |
| 0.000536091 | −0.257161011 | Faecalitalea cylindroides C2250 |
| 0.000528743 | −0.065393049 | Lactobacillus sanfranciscensis TMW 1.1304 C4264 |
| 0.000525582 | −0.129615434 | Absiella sp. AM22-9 C2879 |
| 0.000524183 | 0.15556154 | Streptococcus mitis C5322 |
| 0.00052379 | −0.131464448 | Streptococcus mitis C3901 |
| 0.000521696 | −0.005526656 | Butyricimonas virosa C0441 |
| 0.000521234 | 0.161136825 | Agathobaculum sp. Marseille-P7918 C3297 |
| 0.000520468 | 0.079408986 | Bacteroides intestinalis C0161 |
| 0.000517736 | −0.007357649 | Senegalimassilia sp. KGMB04484 C1994 |
| 0.000515789 | 0.116997696 | Anaeromassilibacillus sp. An172 C2773 |
| 0.000513282 | −0.22188977 | Anaeromassilibacillus sp. Marseille-P4683 C3061 |
| 0.000507316 | −0.160416981 | Clostridium sp. Marseille-P3244 C3177 |
| 0.00050396 | 0.078131194 | Rothia mucilaginosa C3456 |
| 0.000501417 | 0.027192943 | Candidatus Methanomassiliicoccus intestinalis Issoire- |
| | | Mx1 C4599 |
| 0.000499738 | 0.0174744 | Anaerostipes sp. 494a C2731 |
| 0.000498341 | −0.029178099 | Paraeggerthella hongkongensis C1982 |
| 0.000496569 | −0.032045271 | Lactococcus garvieae C6016 |
| 0.000494032 | 0.057726242 | Eubacterium sp. AF19-12LB C2907 |
| 0.000491168 | 0.033329345 | Lachnospiraceae bacterium oral taxon 096 C2846 |
| 0.000491138 | −0.14106364 | Prevotella intermedia C0255 |
| 0.000483914 | 0.076399152 | Bacteroides sp. OM05-12 C1216 |
| 0.000478931 | −0.12999452 | Propionibacterium freudenreichii C3941 |
| 0.000478583 | −0.216633597 | Oxalobacter formigenes C5820 |
| 0.000473254 | 0.13675923 | Eubacterium sp. ER2 C2579 |
| 0.000472977 | −0.15732306 | Alistipes indistinctus C0222 |
| 0.00047013 | −0.01796799 | Traorella massiliensis C3119 |
| 0.000463894 | −0.134877322 | Weissella cibaria C5172 |
| 0.000461977 | 0.043780038 | Prevotella pleuritidis C0414 |
| 0.000461965 | 0.379126295 | Citrobacter sp. FDAARGOS_156 C5320 |
| 0.000458829 | −0.103085248 | [Collinsella] massiliensis C1944 |
| 0.000455848 | −0.216482839 | Alloscardovia omnicolens C0021 |
| 0.000454098 | −0.101700886 | Bacteroides ilei C1793 |
| 0.000452132 | 0.27056853 | Dialister sp. Marseille-P5638 C3282 |
| 0.000447852 | 0.21438821 | Christensenella massiliensis C3223 |
| 0.000446473 | 0.089598965 | Bacteroides cutis C1215 |
| 0.000442533 | −0.125406142 | Prevotella sp. P4-51 C0876 |
| 0.0004413 | 0.013913335 | Bacteroides coprosuis DSM 18011 C0203 |
| 0.000440392 | 0.228587525 | Lachnoclostridium phocaeense C3180 |
| 0.000438656 | 0.057515059 | Ruminococcus bromii C2818 |
| 0.000435684 | 0.170474597 | Prevotella copri C0142 |
| 0.000434472 | 0.278015777 | Enterobacter kobei C4431 |
| 0.000430769 | 0.15735214 | Clostridioides difficile C2586 |
| 0.000429829 | 0.275828056 | Collinsella phocaeensis C2002 |
| 0.000427367 | 0.069690121 | Muribaculaceae bacterium Isolate-102 (HZI) C1306 |
| 0.000425447 | 0.254830162 | [Clostridium] scindens C2446 |
| 0.000425124 | 0.07985737 | Enterobacter roggenkampii C4889 |
| 0.000424989 | 0.039030901 | Erysipelatoclostridium sp. AM42-17 C2927 |
| 0.000422993 | −0.009708183 | Weissella confusa C6837 |
| 0.000421896 | −0.083440639 | Bacteroides fragilis C0140 |
| 0.000421212 | 0.151608309 | Anaerotruncus massiliensis C2969 |
| 0.00041345 | −0.066234762 | Parabacteroides goldsteinii C0282 |
| 0.000409746 | −0.008970984 | Anaerotruncus sp. AF02-27 C2916 |
| 0.000408594 | −0.076490222 | Akkermansia sp. KLE1605 C1918 |
| 0.000408035 | −0.013734886 | Butyricimonas sp. Marseille-P3923 C1885 |
| 0.000404935 | −0.12049091 | Prevotella buccalis C0169 |
| 0.00040246 | 0.105160049 | Merdimonas faecis C2715 |
| 0.000402431 | −0.118683458 | Streptococcus suis C3679 |
| 0.000399456 | 0.167898284 | Klebsiella oxytoca C5296 |
| 0.00039537 | 0.140861068 | Colibacter massiliensis C3075 |
| 0.000394389 | −0.099002939 | Leclercia sp. W6 C6193 |
| 0.000389717 | −0.047195465 | Bifidobacterium pseudolongum C0023 |
| 0.000385746 | 0.137224213 | Clostridiaceae bacterium OM02-2AC C2883 |
| 0.000376658 | −0.006154069 | Odoribacter splanchnicus C0185 |
| 0.000376547 | 0.132471129 | Lactobacillus crispatus C3942 |
| 0.000372155 | 0.077170538 | Clostridium liquoris C2835 |
| 0.000371344 | −0.013289801 | Prevotella shahii C0456 |
| 0.000369066 | 0.058668971 | Prevotella buccae C0148 |
| 0.000368916 | −0.151810317 | Carnobacterium divergens C5502 |
| 0.000365348 | 0.037280739 | Intestinimonas massiliensis C3302 |
| 0.000362723 | 0.096694087 | Megasphaera sp. MJR8396C C2669 |
| 0.000362664 | −0.209757142 | Lactococcus lactis C3326 |
| 0.000359275 | 0.013743499 | Ruminococcus gauvreauii DSM 19829 C2421 |
| 0.000354446 | −0.067786957 | Megasphaera sp. NM10 C2382 |
| 0.000354236 | −0.101587608 | Lactobacillus sakei C3886 |
| 0.000349708 | 0.052923252 | Fusobacterium varium C2031 |
| 0.000349377 | 0.12801912 | Raoultella ornithinolytica C4582 |
| 0.000342204 | −0.224210946 | Clostridium sp. CL-2 C2570 |
| 0.000339415 | −0.018984193 | Schaalia odontolytica C6913 |
| 0.000336634 | 0.085134208 | [Clostridium] aminophilum C2554 |
| 0.000318155 | 0.079087407 | Escherichia sp. E4742 C6917 |
| 0.000317742 | −0.111845972 | Porphyromonas sp. COT-290 OH860 C0549 |
| 0.000316438 | −0.129465239 | Criibacterium bergeronii C2703 |
| 0.00031524 | −0.151761323 | Gardnerella vaginalis C0008 |
| 0.000313466 | 0.093860399 | Citrobacter freundii complex sp. CFNIH3 C5883 |
| 0.00031154 | −0.030174898 | Veillonella sp. S13053-19 C2226 |
| 0.000305314 | −0.019154943 | Enterococcus casseliflavus C4021 |
| 0.000301412 | −0.028685455 | Clostridium paraputrificum C2404 |
| 0.000301347 | 0.135067509 | Citrobacter amalonaticus C5315 |
| 0.000299201 | 0.056396549 | Peptoniphilus harei C2229 |
| 0.000295876 | 0.105606587 | Lactobacillus reuteri C3427 |
| 0.00029558 | −0.087470002 | Prevotella bivia C0170 |
| 0.00029533 | 0.2944697 | Massilimicrobiota sp. An134 C2756 |
| 0.000292461 | −0.16360222 | Clostridium celatum DSM 1785 C2336 |
| 0.000290231 | −0.107393237 | Eubacterium saphenum ATCC 49989 C2183 |
| 0.000289466 | 0.098825501 | Caproiciproducens galactitolivorans C3034 |
| 0.000283388 | 0.088913554 | Peptococcus niger C3096 |
| 0.000281338 | −0.199624188 | Bacteroides sp. Marseille-P3684 C1903 |
| 0.000280597 | −0.35259961 | [Eubacterium] rectale C2102 |
| 0.000278229 | −0.123327354 | Hungatella hathewayi C2277 |
| 0.000275274 | 0.032280996 | Raoultibacter timonensis C2015 |
| 0.000274761 | 0.026049476 | Bifidobacterium minimum C0024 |
| 0.000274208 | −0.250305123 | Slackia isoflavoniconvertens C1981 |
| 0.000272806 | −0.148295608 | Prevotella sp. 109 C0642 |
| 0.000271138 | 0.085385438 | Bacteroides ndongoniae C1721 |
| 0.000270351 | 0.096334331 | Sanguibacteroides justesenii C0594 |
| 0.000268105 | −0.092100556 | Enterococcus sp. M190262 C4628 |
| 0.000264389 | 0.028275689 | Candidatus Soleaferrea massiliensis AP7 C2589 |
| 0.000258394 | 0.095952069 | Fusobacterium mortiferum C2024 |
| 0.000257776 | −0.123638868 | Mitsuokella jalaludinii C2546 |
| 0.000256938 | −0.044689114 | Haemophilus pittmaniae C7263 |
| 0.000256376 | 0.057471563 | Citrobacter koseri C3675 |
| 0.000255931 | 0.098498867 | Staphylococcus epidermidis C3349 |
| 0.000255753 | −0.157103426 | Lachnotalea sp. AF33-28 C2930 |
| 0.000249354 | 0.103829306 | Streptococcus troglodytae C6006 |
| 0.000247989 | −0.04348123 | Eubacterium nodatum ATCC 33099 C2463 |
| 0.000237849 | 0.136436955 | Bacteroides acidifaciens C0454 |
| 0.000235895 | 0.025076982 | Cloacibacillus porcorum C5498 |
| 0.000234449 | 0.207586523 | Desulfovibrio fairfieldensis C5303 |
| 0.000232439 | 0.06389105 | Citrobacter amalonaticus Y19 C5026 |
| 0.000231523 | 0.21351164 | Frisingicoccus caecimuris C3012 |
| 0.000229189 | 0.116831596 | Streptococcus equinus C4630 |
| 0.000224376 | −0.071274749 | Enterobacter ludwigii C4314 |
| 0.000223221 | −0.001425117 | Lachnospira multipara C2406 |
| 0.000219577 | −0.252448758 | Comamonas kerstersii C5760 |
| 0.000215028 | −0.201289125 | Odoribacter sp. AF15-53 C1228 |
| 0.000212884 | 0.129978944 | Clostridium ventriculi C2645 |
| 0.000212879 | 0.012267554 | Prevotella denticola C0190 |
| 0.00021254 | −0.090029408 | Acidaminococcus timonensis C3121 |
| 0.000209014 | 0.081821172 | Pediococcus acidilactici C5564 |
| 0.00020599 | −0.091958501 | Parabacteroides gordonii C0394 |
| 0.000204627 | −0.03219179 | Salmonella bongori C4344 |
| 0.000201044 | 0.046058989 | Corynebacterium argentoratense DSM 44202 C4728 |
| 0.000195048 | −0.148177716 | Ruminococcus sp. Marseille-P6503 C3293 |
| 0.000193863 | 0.115675916 | Veillonella atypica C2224 |
| 0.000191688 | 0.075560921 | Clostridium neonatale C2656 |
| 0.000191566 | 0.059627079 | Hafnia paralvei C5321 |
| 0.000187799 | 0.004958554 | Ruminococcus bromii C3091 |
| 0.000187592 | 0.108114105 | Megasphaera micronuciformis F0359 C2190 |
| 0.000185989 | 0.049809679 | Hafnia alvei C4732 |
| 0.000184299 | 0.072305952 | Clostridium sp. Marseille-P8228 C3298 |
| 0.000182455 | 0.081144244 | Salmonella enterica C3691 |
| 0.000182401 | 0.040354086 | Prevotella maculosa C0236 |
| 0.000180958 | 0.045550681 | Tetragenococcus halophilus C4414 |
| 0.000180446 | 0.155453018 | [Clostridium] cocleatum C2817 |
| 0.000175141 | 0.003197966 | Ruminococcus flavefaciens C3174 |
| 0.000175125 | 0.088990805 | Clostridium sp. CL-6 C2568 |
| 0.000173291 | −0.017099749 | Prevotella sp. P5-125 C0597 |
| 0.000169963 | −0.057233209 | Pseudomonas fragi C5503 |
| 0.00016916 | −0.248823705 | Leuconostoc gelidum JB7 C4451 |
| 0.00016589 | 0.065183802 | Cronobacter sakazakii C3665 |
| 0.00016331 | −0.208923621 | Megasphaera elsdenii C2304 |
| 0.000161384 | 0.067558754 | Klebsiella oxytoca C5056 |
| 0.000161379 | 0.13837813 | Lactobacillus helveticus C3606 |
| 0.000159676 | −0.003463017 | Pediococcus pentosaceus C3572 |
| 0.000157298 | 0.144136167 | Enterobacter hormaechei C4773 |
| 0.000155828 | −0.260724092 | Roseburia sp. AM59-24XD C2936 |
| 0.000151336 | −0.292938975 | Lactobacillus delbrueckii C3568 |
| 0.000141557 | 0.076327611 | Prevotella salivae C0180 |
| 0.000131281 | 0.143665959 | Lactobacillus amylovorus C4089 |
| 0.000130941 | −0.047422488 | Lactobacillus ruminis ATCC 27782 C4263 |
| 0.000130595 | −0.04481037 | Paraclostridium bifermentans C2432 |
| 0.000129911 | 0.167682779 | Escherichia albertii C4681 |
| 0.000127495 | 0.04633969 | Enterococcus durans C5114 |
| 0.000127484 | 0.072529092 | Cellulosilyticum sp. WCF-2 C2221 |
| 0.000123473 | 0.173686087 | Clostridiales bacterium S5-A14a C2574 |
| 0.000122727 | −0.074297589 | Blautia wexlerae C2171 |
| 0.000121299 | −0.053122344 | Methanosphaera stadtmanae DSM 3091 C3505 |
| 0.000120188 | 0.119050783 | Clostridium sp. MSTE9 C2303 |
| 0.000120039 | −0.052843577 | Clostridium disporicum C2646 |
| 0.000116659 | 0.080030593 | Lactobacillus johnsonii C3366 |
| 0.000113997 | 0.104093107 | Serratia marcescens C4687 |
| 0.000113245 | −0.00308721 | Prevotella amnii C0171 |
| 0.000107199 | −0.022568473 | Cronobacter condimenti 1330 C5129 |
| 0.000104701 | 0.000252647 | Ruminococcaceae bacterium CPB6 C2750 |
| 0.000104084 | 0.066800683 | Veillonella ratti C2991 |
| 0.000102394 | 0.152599321 | Bacteroides paurosaccharolyticus JCM15092 C0457 |
| 9.37347E−05 | 0.174241239 | Lactobacillus gasseri C3569 |
| 8.56015E−05 | 0.059945469 | [Clostridium] hylemonae C2157 |
| 7.75294E−05 | 0.1191171 | Citrobacter amalonaticus C5318 |
| 7.55257E−05 | 0.068345197 | Bacteroides sp. KCTC15687 C1337 |
| 6.75319E−05 | 0.006391049 | Lactococcus garvieae C4388 |
| 6.59076E−05 | 0.120223702 | Faecalicoccus pleomorphus C2383 |
| 6.45031E−05 | 0.097753343 | Lactobacillus animalis C6895 |
| 5.21062E−05 | 0.149698537 | Anaerostipes rhamnosivorans C3039 |
| 4.42633E−05 | −0.007497948 | Enterobacter bugandensis C5325 |
| 4.37847E−05 | 0.032643624 | Lactobacillus mucosae LM1 C4338 |
| 4.32409E−05 | 0.065872962 | Bacteroides propionicifaciens C0324 |
| 0 | 0.078372213 | Streptococcus sobrinus C6344 |
| 0 | −0.064034551 | Ruminococcaceae bacterium D5 C3161 |
| 0 | 0.015908673 | Ruminococcus albus C3136 |
| 0 | 0.070235779 | Selenomonas noxia C2179 |
| 0 | 0.102015151 | Citrobacter werkmanii C4750 |
| 0 | 0.106931981 | Providencia rettgeri C6875 |
| 0 | −0.08278651 | Anaerococcus lactolyticus C2159 |
| 0 | 0.026978526 | Ruminococcus sp. FC2018 C2499 |
| 0 | 0.040473615 | Robinsoniella peoriensis C2512 |
| 0 | −0.153859627 | Megasphaera hexanoica C2664 |
| 0 | 0.005437415 | Atlantibacter hermannii C7332 |
| 0 | −0.050219427 | Megasphaera sp. AM44-1BH C2918 |
| 0 | 0.013360056 | Clostridium sp. 12(A) C2475 |
| 0 | −0.075062059 | Eggerthella sinensis C1979 |
| 0 | 0.029503909 | Proteus vulgaris C6084 |
| 0 | 0.020972769 | Plautia stall symbiont C4087 |
| 0 | −0.009219528 | Bacteroides graminisolvens C0392 |
| 0 | 0.034902834 | Providencia rettgeri C4489 |
| 0 | −0.072959896 | Candidatus Ishikawaella capsulata Mpkobe C4922 |
| 0 | −0.060674729 | secondary endosymbiont of Ctenarytaina eucalypti C4438 |
| 0 | 0.000740595 | Shimwellia blattae C4368 |
| 0 | 0.042068637 | Bacteroides reticulotermitis JCM 10512 C0437 |
| 0 | 0.134402606 | Proteus mirabilis C3929 |
| 0 | 0.085291723 | Peptoclostridium sp. AF21-18 C2156 |
| 0 | 0.071303376 | Bacteroidales bacterium KA00251 C0708 |
| 0 | 0.044896419 | Klebsiella sp. PO552 C5864 |
| 0 | −0.020350527 | Cronobacter universalis NCTC 9529 C5126 |
| 0 | 0.042060758 | Lelliottia jeotgali C5960 |
| 0 | 0.010010498 | Pseudomonas balearica DSM 6083 C4912 |
| 0 | 0.069859304 | Fusobacterium nucleatum C2036 |
| 0 | −0.098855648 | Mitsuokella sp. AF21-1AC C2899 |

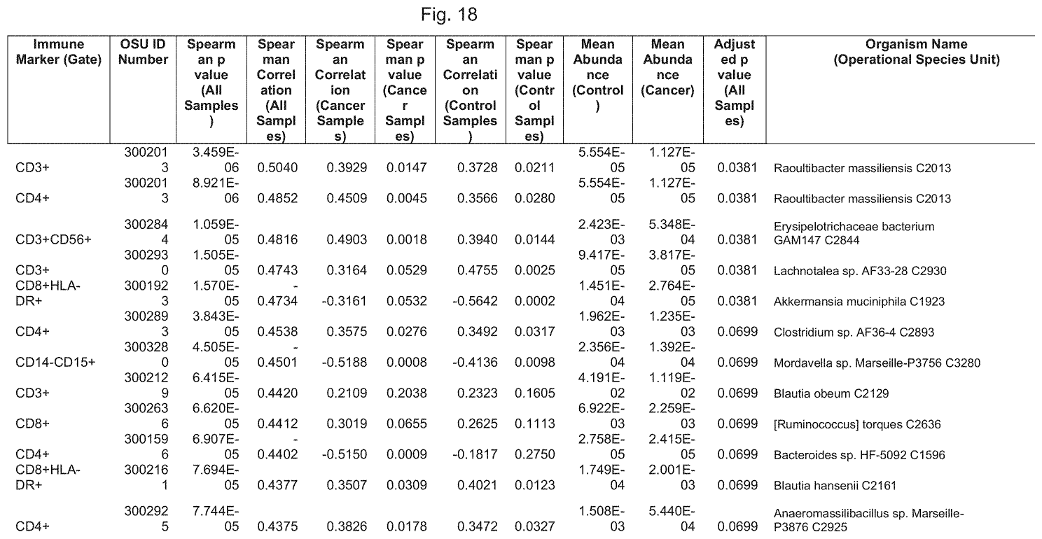

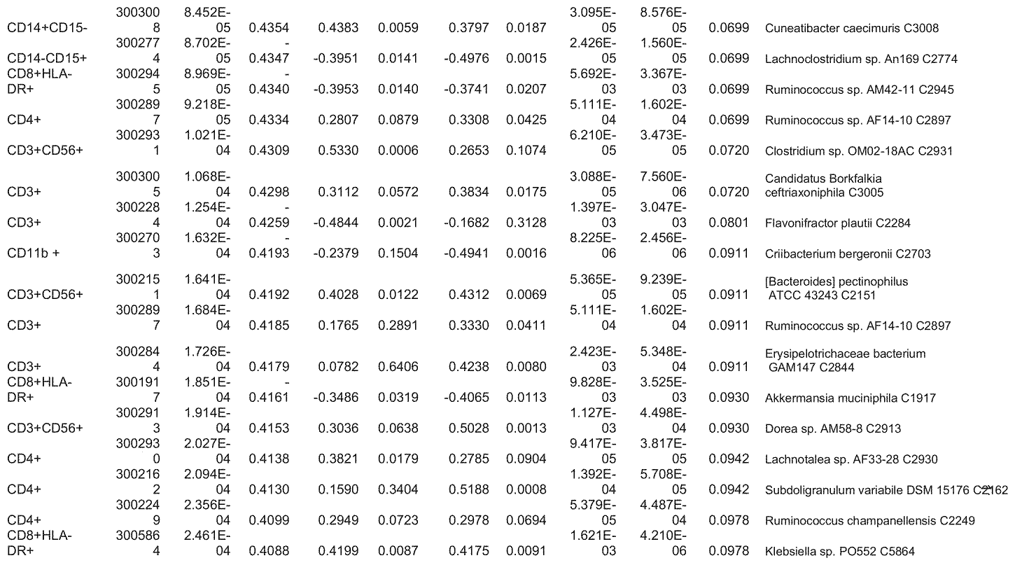

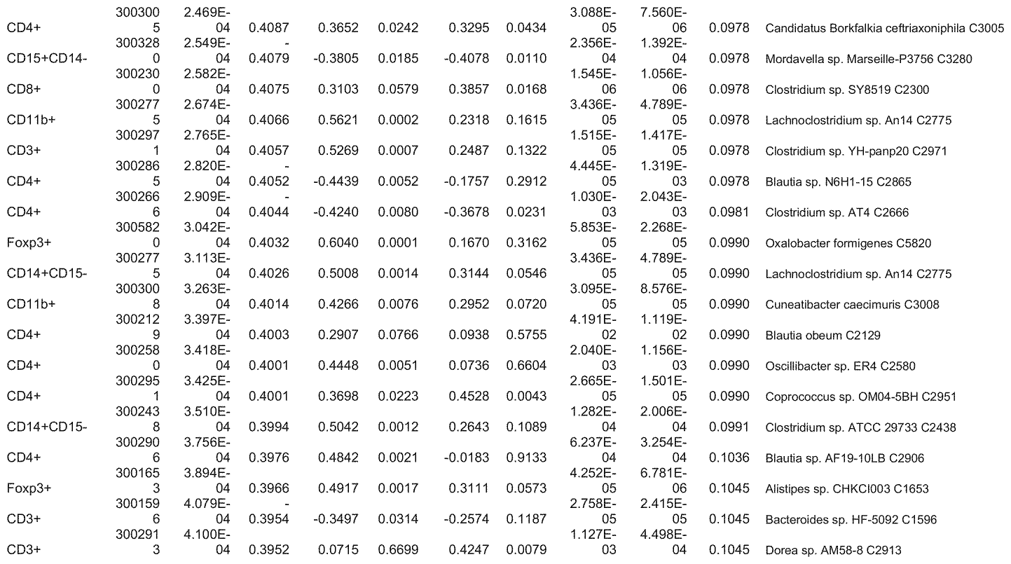

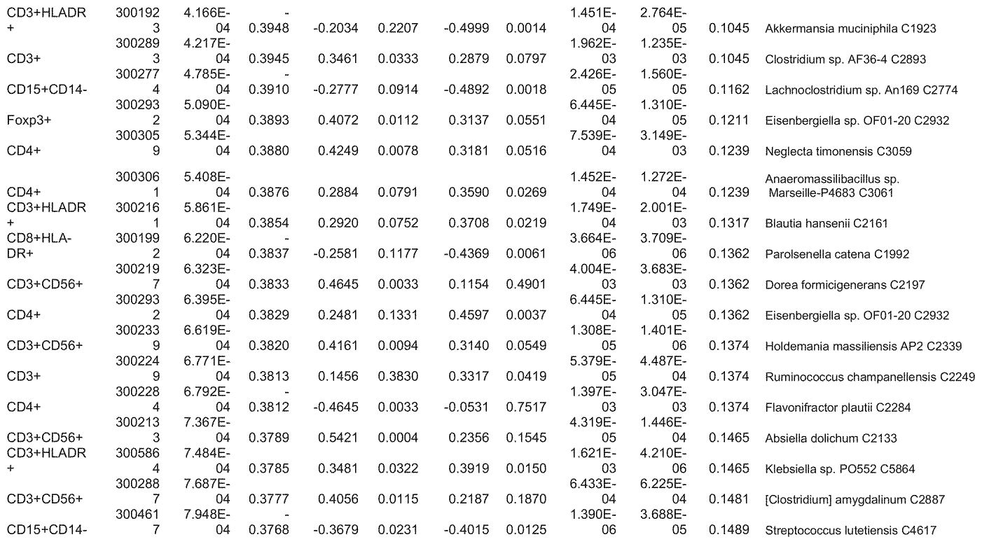

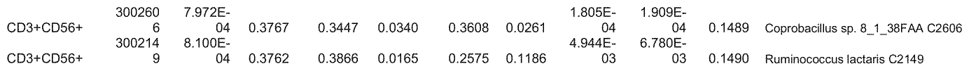

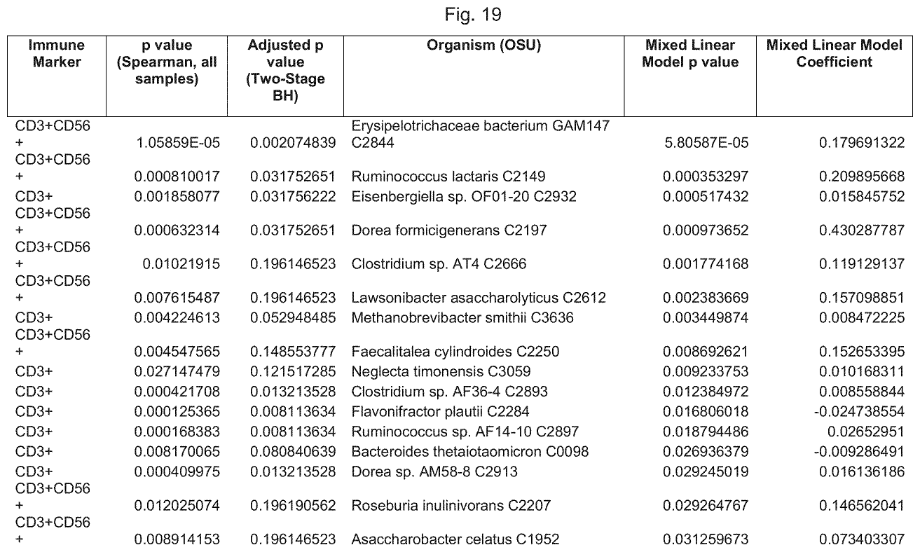

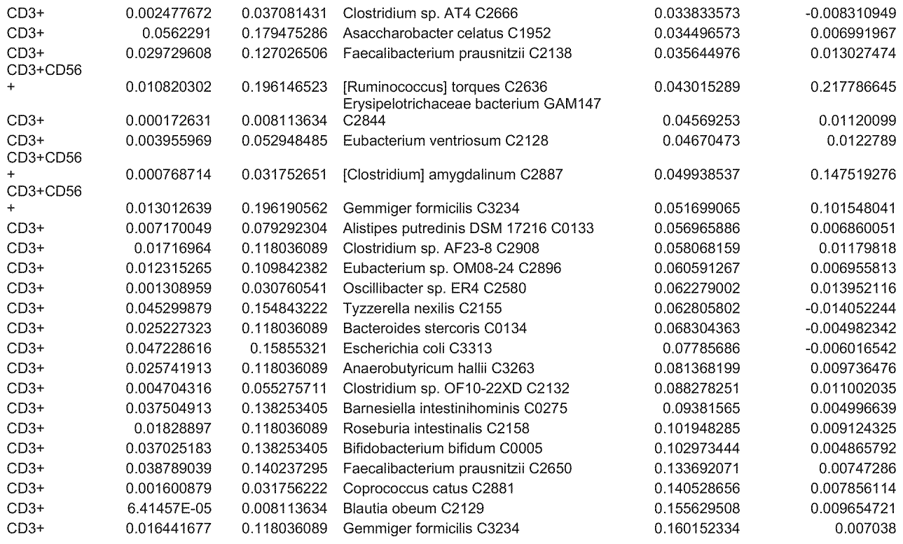

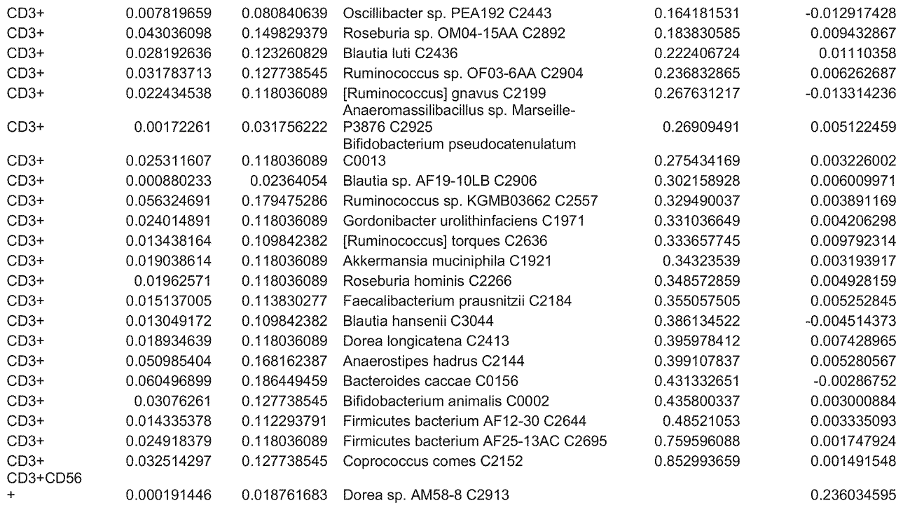

Table 5, illustrated as FIG. 18. Flow cytometry was performed on 38 cancer blood samples and 38 control blood samples, along with corresponding whole genome sequencing and classification. All operational species unit (OSU) abundances were correlated against a suite of immune markers (CD11b+, CD14+CD15−, CD14-CD15+, CD15+CD14−, CD15-CD14+, CD3+, CD3+CD56+, CD3+HLADR+, CD3-CD56+, CD3-HLA-DR+, CD3-HLA-DRlow, CD4+, CD4+HLA-DR+, CD8+, CD8+HLA-DR+, Foxp3+). Correlations and p values were computed on all the samples, or on a subset of samples consisting of just control samples or just cancer samples. The p values obtained from all the samples were filtered using a two-stage Benjamini-Hochberg procedure and correlated with an adjusted p value below 0.15 are reported.
Table 6, illustrated in FIG. 19. Flow cytometry was performed on 38 cancer blood samples and 38 control blood samples, along with corresponding whole genome sequencing and classification. All operational species units (OSUs) were correlated against the CD3+ and CD3+CD56+ immune markers (as a subset of CD45+) using a Spearman rank correlation. Adjusted p values were computed using a two-stage Benjamini-Hochberg procedure for each immune marker, and correlations with an adjusted p value below 0.2 are retained. The retained correlations were further vetted using a linear mixed model that accounts for a random effect induced by group (cancer vs. control). The logarithm of the OSU abundance was used as the input to the model. For CD3+CD56+, the logarithm of the immune marker proportion was used as the output of the mixed model. The mixed model p values and coefficients are reported.

TABLE 7
Whole genome sequencing was performed on the initial time point fecal samples from subjects undergoing
cancer immunotherapy and the reads were classified and abundance of each operational species unit
was estimated computationally. Operational species unit abundances were correlated to response
to therapy using a score of 2 for complete response, 1 for partial response, 0 for no response,
using the Spearman rank correlation. Correlations with a p value below 0.15 are reported.

| Mean Abundance | p value | Spearman | Organism | Adjusted p value |
|---|---|---|---|---|
| (All Samples) | (Spearman rank) | Correlation | (Operational Species Unit) | (Two Stage BH) |
| 0.004824863 | 0.009065354 | −0.460919277 | Bacteroides barnesiae C0323 | 0.50270646 |
| 0.001854242 | 0.011550331 | −0.447714196 | Streptococcus mutans C3345 | 0.50270646 |
| 0.002592642 | 0.013008588 | −0.441044685 | Lactobacillus fermentum C3433 | 0.50270646 |
| 0.003900899 | 0.01697159 | −0.425648294 | Bacteroides heparinolyticus C1005 | 0.50270646 |
| 0.011114361 | 0.020991328 | 0.412834447 | Bacteroides coprosuis DSM 18011 C0203 | 0.50270646 |
| 0.001347612 | 0.021974899 | −0.410011686 | Blautia obeum C2901 | 0.50270646 |
| 0.005138808 | 0.022206972 | −0.409360874 | Streptococcus vestibularis C7338 | 0.50270646 |
| 0.004109069 | 0.028915901 | −0.392598094 | Streptococcus thermophilus C3480 | 0.50270646 |
| 0.002625559 | 0.029553117 | 0.391177567 | Bacteroides eggerthii C0137 | 0.50270646 |
| 0.00180933 | 0.029570968 | −0.391138132 | Streptococcus sp. HSISS2 C4629 | 0.50270646 |
| 0.006421035 | 0.045485127 | −0.361828833 | Bacteroides coprocola C0136 | 0.702951961 |
| 0.00479161 | 0.066985005 | −0.333215846 | Lachnospira pectinoschiza C2649 | 0.884186581 |
| 0.001357573 | 0.067614268 | −0.332494913 | Lactobacillus paragasseri C5843 | 0.884186581 |
| 0.002942156 | 0.074018907 | −0.325438908 | Escherichia coli C3313 | 0.894382098 |
| 0.001499661 | 0.089187848 | −0.310437419 | Intestinibacter bartlettii C2141 | 0.894382098 |
| 0.001315245 | 0.090931568 | −0.308841524 | Lactococcus lactis C3409 | 0.894382098 |
| 0.000593797 | 0.093500218 | 0.306532546 | Anaerotignum lactatifermentans C2790 | 0.894382098 |
| 0.001096895 | 0.100936329 | −0.300108932 | Bifidobacterium dentium C0003 | 0.894382098 |
| 0.001297862 | 0.101670448 | 0.2994944 | Odoribacter splanchnicus C0185 | 0.894382098 |
| 0.002123253 | 0.113189533 | −0.290262241 | Faecalimonas umbilicata C2244 | 0.894382098 |
| 0.014086171 | 0.120986249 | 0.284404931 | Faecalibacterium prausnitzii C2138 | 0.894382098 |
| 0.001420926 | 0.123671567 | 0.282452495 | Tyzzerella nexilis C2155 | 0.894382098 |
| 0.000841219 | 0.131047516 | 0.277245997 | Clostridiales bacterium CCNA10 C2953 | 0.894382098 |
| 0.001049951 | 0.132465355 | −0.276270029 | Clostridium disporicum C2479 | 0.894382098 |
| 0.000534773 | 0.1330099 | 0.275897229 | Gordonibacter pamelaeae C1937 | 0.894382098 |

TABLE 8
Operational species units (OSUs) with a mean abundance of at least 0.05% with significant differences between cancer and
control cohorts for inclusion into the therapeutic. For each OSU, CD3+ and CD3+ CD56+ correlations are
included in the table as per the linear mixed model analysis or set to zero if the mixed model correlation is negative
or if the Spearman correlation was not significant enough to necessitate mixed model analysis. The cancer and control
fold change, CD3+ correlation, and CD3+ CD56+ correlation for each OSU were converted to percentile scores,
and a combined score for each OSU was generated as the geometric mean of each of the three percentiles.
Table 8

| | | | CD3+ | CD3+ CD56+ | |
|---|---|---|---|---|---|
| p value | | | Correlation | Correlation | |
| Control vs Cancer | log10 Fold Change | Organism Name | (Spearman, if | (Spearman, if | |
| (Mann Whitney U) | (Cancer/ Control) | (Operational Species Unit) | significant) | significant) | Total Score |
| 1.13356E−08 | −0.764382216 | Erysipelotrichaceae bacterium GAM147 C2844 | 0.417881066 | 0.481640465 | 99.3759725 |
| 0.000236114 | −0.432405099 | Dorea sp. AM58-8 C2913 | 0.395242652 | 0.415256323 | 94.53854775 |
| 0.000111496 | −0.304525941 | [Ruminococcus] torques C2636 | 0.282433356 | 0.290799727 | 81.68743735 |
| 4.96202E−05 | −0.504914016 | Blautia obeum C2129 | 0.441968558 | 0 | 75.35264806 |
| 1.19211E−05 | −0.565340143 | Firmicutes bacterium AF12-30 C2644 | 0.279890636 | 0 | 73.10872098 |
| 3.1747E−07 | −0.415683892 | Blautia sp. AF19-10LB C2906 | 0.3738098 | 0 | 71.5962122 |
| 0.016231058 | −0.392581823 | Clostridium sp. AF36-4 C2893 | 0.39447635 | 0 | 71.56015136 |
| 3.36506E−05 | −0.474788291 | Faecalibacterium prausnitzii C2184 | 0.277732589 | 0 | 71.19231957 |
| 3.45381E−06 | −0.557690435 | Ruminococcus sp. OF03-6AA C2904 | 0.246561859 | 0 | 69.34836951 |
| 1.9624E−05 | −0.436129729 | Dorea longicatena C2413 | 0.268680793 | 0 | 69.18884624 |
| 0.013509112 | −0.452532206 | Bifidobacterium pseudocatenulatum C0013 | 0.256499594 | 0 | 69.12262094 |
| 0.008426878 | −0.517722756 | Bifidobacterium bifidum C0005 | 0.239694858 | 0 | 68.55170178 |
| 1.77058E−05 | −0.449283525 | Coprococcus comes C2152 | 0.245550239 | 0 | 67.24353586 |
| 0.006074794 | −0.502954606 | Ruminococcus sp. KGMB03662 C2557 | 0.219878468 | 0 | 66.11567283 |
| 0.00457584 | −0.312675608 | Clostridium sp. OF10-22XD C2132 | 0.320929597 | 0 | 65.97044298 |
| 0.003783812 | −0.358288898 | Faecalibacterium prausnitzii C2138 | 0.249514696 | 0 | 65.6229349 |
| 0.015331343 | −0.34579043 | Firmicutes bacterium AF25-13AC C2695 | 0.257170198 | 0 | 65.61067466 |
| 0.01271148 | −0.27637531 | Coprococcus catus C2881 | 0.35595352 | 0 | 65.4138845 |
| 0.000860995 | −0.417870861 | Faecalibacterium prausnitzii C2650 | 0.237566644 | 0 | 65.21900583 |
| 0.51444269 | −0.130545436 | Gemmiger formicilis C3234 | 0.27442242 | 0.283691046 | 64.00839092 |
| 0.014929144 | −0.247491324 | Oscillibacter sp. ER4 C2580 | 0.362077922 | 0 | 63.36416394 |
| 0.001048844 | −0.353929055 | Anaerostipes hadrus C2144 | 0.224716336 | 0 | 63.19608844 |
| 0.019540986 | −0.319221468 | Ruminococcus lactaris C2149 | 0 | 0.37621326 | 61.28393922 |
| 0.013186687 | −0.224153342 | Eubacterium ventriosum C2128 | 0.32683527 | 0 | 59.96015726 |
| 0.002439804 | −0.251539605 | Blautia luti C2436 | 0.251838688 | 0 | 59.70580459 |
| 0.039249769 | −0.240687636 | Anaerobutyricum hallii C3263 | 0.255775803 | 0 | 58.66605169 |
| 0.018826044 | −0.257161011 | Faecalitalea cylindroides C2250 | 0 | 0.322093794 | 58.53674188 |
| 0.03257254 | −0.230812001 | Dorea formicigenerans C2197 | 0 | 0.383267259 | 56.61344825 |
| 0.29746277 | −0.106066732 | Asaccharobacter celatus C1952 | 0.219961719 | 0.298085866 | 55.95335448 |
| 0.386245537 | −0.224535064 | Barnesiella intestinihominis C0275 | 0.239107807 | 0 | 55.71314544 |
| 0.717556152 | −0.160088535 | Alistipes putredinis DSM 17216 C0133 | 0.306064417 | 0 | 53.10583588 |
| 3.44484E−05 | −0.516206872 | Dorea longicatena C2131 | 0 | 0 | 52.8235779 |
| 0.005503739 | −0.476696467 | Collinsella aerofaciens C1933 | 0 | 0 | 52.30053782 |
| 7.0925E−05 | −0.442770588 | Dorea sp. OM07-5 C2890 | 0 | 0 | 51.58644796 |
| 0.08814635 | −0.14769711 | Clostridium sp. AF23-8 C2908 | 0.272672591 | 0 | 51.08317009 |
| 0.00646676 | −0.421426117 | Anaerobutyricum hallii C2206 | 0 | 0 | 51.03761433 |
| 0.026898361 | −0.165551333 | [Clostridium] amygdalinum C2887 | 0 | 0.37771702 | 50.67666817 |
| 0.577485114 | −0.11819143 | Eubacterium sp. OM08-24 C2896 | 0.285831465 | 0 | 50.55687075 |
| 0.009817398 | −0.391815999 | Romboutsia timonensis C3123 | 0 | 0 | 50.28695213 |
| 0.011792583 | −0.36246911 | Faecalibacterium prausnitzii C2651 | 0 | 0 | 50.09574515 |
| 0.004344805 | −0.352856347 | Ruminococcus callidus C2440 | 0 | 0 | 49.51318366 |
| 0.016312939 | −0.35259961 | [Eubacterium] rectale C2102 | 0 | 0 | 49.31591789 |
| 0.001239483 | −0.344627962 | Blautia sp. TF11-31AT C2841 | 0 | 0 | 48.91658119 |
| 0.233152423 | −0.335185925 | Bifidobacterium adolescentis C0001 | 0 | 0 | 48.71444425 |
| 0.019421399 | −0.326716726 | Subdoligranulum sp. APC924/74 C2870 | 0 | 0 | 48.51061574 |
| 0.035937114 | −0.32340108 | Ruminococcus sp. AM42-11 C2945 | 0 | 0 | 48.30505982 |
| 0.00812493 | −0.322415354 | Blautia sp. KGMB01111 C3003 | 0 | 0 | 48.09773941 |
| 0.097361497 | −0.321124776 | Clostridium disporicum C2479 | 0 | 0 | 47.88861616 |
| 0.117266616 | −0.306056064 | Bacteroides heparinolyticus C1005 | 0 | 0 | 47.25002508 |
| 0.038561827 | −0.305064647 | Firmicutes bacterium TM09-10 C2909 | 0 | 0 | 47.0332788 |
| 0.269625863 | −0.114661776 | Bifidobacterium animalis C0002 | 0.248008991 | 0 | 47.00940403 |
| 0.188811422 | −0.270611264 | [Eubacterium] eligens C2123 | 0 | 0 | 46.37075 |
| 0.023532312 | −0.26283664 | Clostridium sp. AM49-4BH C2934 | 0 | 0 | 46.14564573 |
| 0.555562154 | −0.10501558 | Roseburia hominis C2266 | 0.267204375 | 0 | 46.03382224 |
| 0.187155428 | −0.260724092 | Roseburia sp. AM59-24XD C2936 | 0 | 0 | 45.91832359 |
| 0.326490078 | −0.116696596 | Roseburia inulinivorans C2207 | 0 | 0.286753247 | 45.8451976 |
| 0.001936194 | −0.255666512 | Faecalibacterium sp. AF28-13AC C2810 | 0 | 0 | 45.45680166 |
| 0.005147868 | −0.246601387 | Agathobaculum butyriciproducens C2850 | 0 | 0 | 44.74642259 |
| 0.009668016 | −0.242001524 | Faecalibacterium prausnitzii C2863 | 0 | 0 | 44.50454531 |
| 0.23748252 | −0.094755491 | Anaeromassilibacillus sp. Marseille-P3876 C2925 | 0.353693881 | 0 | 44.32727446 |
| 0.553126103 | −0.098634411 | Roseburia intestinalis C2158 | 0.270102529 | 0 | 44.05697948 |
| 0.028683688 | −0.235792289 | Faecalibacterium prausnitzii C2864 | 0 | 0 | 44.01274212 |
| 0.01512723 | −0.229970619 | Firmicutes bacterium AF22-6AC C2933 | 0 | 0 | 43.5096953 |
| 0.299408337 | −0.223572419 | Faecalibacterium prausnitzii C2191 | 0 | 0 | 42.73256973 |
| 0.230520123 | −0.219463054 | Bacteroides finegoldii C0138 | 0 | 0 | 42.46714319 |
| 0.07671217 | −0.209757142 | Lactococcus lactis C3326 | 0 | 0 | 42.1983566 |
| 0.412393028 | −0.209498347 | Bacteroides massiliensis C0310 | 0 | 0 | 41.92610156 |
| 0.004324117 | −0.206044424 | Clostridium sp. AF20-17LB C2921 | 0 | 0 | 41.65026396 |
| 0.000839149 | −0.201826507 | Fusicatenibacter saccharivorans C2643 | 0 | 0 | 41.37072356 |
| 0.171446365 | −0.192935162 | Clostridium sp. AF46-9NS C2891 | 0 | 0 | 41.08735355 |
| 0.190921078 | −0.191454148 | Streptococcus thermophilus C3480 | 0 | 0 | 40.80002 |
| 0.24012289 | −0.191448288 | [Clostridium] spiroforme C2146 | 0 | 0 | 40.50858134 |
| 0.238875443 | −0.189503492 | Holdemanella biformis C2160 | 0 | 0 | 40.21288772 |
| 0.350722809 | −0.18109626 | Bifidobacterium longum C0000 | 0 | 0 | 39.91278036 |
| 0.092953142 | −0.093698754 | Roseburia sp. OM04-15AA C2892 | 0.232754614 | 0 | 39.82976685 |
| 0.511730372 | −0.167126002 | Firmicutes bacterium AF36-3BH C2905 | 0 | 0 | 39.60809076 |
| 0.002091197 | −0.163492912 | Clostridium sp. AM18-55 C2845 | 0 | 0 | 38.98423732 |
| 0.044817697 | −0.161787704 | Ruminococcus sp. AF31-8BH C2903 | 0 | 0 | 38.66468002 |
| 0.163286482 | −0.158568078 | Bacteroides stercoris C0134 | 0 | 0 | 38.00921984 |
| 0.196410058 | −0.123209619 | Coprococcus eutactus C2642 | 0 | 0 | 36.98143604 |
| 0.645760506 | −0.111096287 | Eisenbergiella tayi C2259 | 0 | 0 | 35.51525914 |
| 0.247286492 | −0.107393237 | Eubacterium saphenum ATCC 49989 C2183 | 0 | 0 | 35.12919314 |
| 0.194954789 | −0.103823837 | Eubacterium ramulus C2442 | 0 | 0 | 33.91686307 |
| 0.072831555 | −0.103576385 | Bacteroides uniformis C0132 | 0 | 0 | 33.49285783 |
| 0.568492801 | −0.100055999 | [Eubacterium] siraeum C2135 | 0 | 0 | 33.05783641 |
| 0.505564788 | −0.096838499 | Intestinibacter bartlettii C2141 | 0 | 0 | 32.15168231 |
| 0.083226778 | −0.087543931 | Blautia obeum C2901 | 0 | 0 | 30.68817687 |
| 0.025598358 | −0.081852178 | Ruminococcus sp. AF24-32LB C2894 | 0 | 0 | 30.16793778 |
| 0.992080795 | −0.077567242 | Megamonas funiformis C2294 | 0 | 0 | 29.629109 |
| 0.933770138 | −0.076490222 | Akkermansia sp. KLE1605 C1918 | 0 | 0 | 29.06993521 |
| 0.386824312 | −0.074691634 | Bacteroides nordii C0263 | 0 | 0 | 28.48837982 |
| 0.085278665 | −0.074297589 | Blautia wexlerae C2171 | 0 | 0 | 27.88205907 |
| 0.330051192 | −0.073609518 | Clostridium sp. TM06-18 C2922 | 0 | 0 | 27.24815505 |
| 0.083262321 | −0.072959896 | Candidatus Ishikawaella capsulata Mpkobe C4922 | 0 | 0 | 26.58329888 |
| 0.683600356 | −0.066234762 | Parabacteroides goldsteinii C0282 | 0 | 0 | 25.88341081 |
| 0.643910037 | −0.061735341 | Alistipes sp. 5CBH24 C0283 | 0 | 0 | 25.14347607 |
| 0.242773051 | −0.052348168 | Lachnospira pectinoschiza C2649 | 0 | 0 | 24.35722212 |
| 0.283452611 | −0.048932599 | Clostridium sp. AF34-13 C2653 | 0 | 0 | 23.5166394 |
| 0.792065697 | −0.04739511 | Catenibacterium mitsuokai DSM 15897 C2204 | 0 | 0 | 22.61124205 |
| 0.75605445 | −0.045467144 | Eubacterium sp. TM06-47 C2917 | 0 | 0 | 21.626875 |
| 0.47084588 | −0.041470873 | Coprococcus eutactus C2140 | 0 | 0 | 20.54367678 |
| 0.903395164 | −0.032159744 | Roseburia faecis C2648 | 0 | 0 | 19.33234001 |
| 0.776663172 | −0.022510129 | Bacteroides faecis C0221 | 0 | 0 | 17.94655471 |
| 0.686897002 | −0.016216198 | Bacteroides sp. OF04-15BH C1226 | 0 | 0 | 16.30552706 |
| 0.77256713 | −0.008006904 | Lawsonibacter asaccharolyticus C2612 | 0 | 0.303877071 | 14.43735499 |
| 0.226827239 | −0.011837149 | Bacteroides fragilis C0096 | 0 | 0 | 14.24418991 |
| 0.804445324 | −0.006154069 | Odoribacter splanchnicus C0185 | 0 | 0 | 0 |

### Machine Learning for Live Biotherapeutic Design

The top 32 scoring organisms from Example 9 (Table 6) is selected for screening in simulated microbial mixes. Each combination of 4 organisms from the 32 is evaluated in silico using the trained machine learning model. For the cancer samples in the model, relative species abundances for the four organisms in the putative mix are increased in silico by a certain amount (here 0.5%). This simulates in silico the physical action of adding microbes to the gut microbiome. Classification is then performed using the machine learning model to estimate the probability that each augmented sample is a cancer sample. The hypothesis is that combinations of microbes that make cancer samples appear more like control samples according to the model are better candidates for therapeutic mixes. Each putative mix is scored by its mean predicted cancer probability across all the augmented cancer samples, with lower mean predicted cancer probabilities corresponding to notionally better therapeutic candidates. The top 30 exemplary live biotherapeutic compositions (exemplary microbial combinations) are then validated experimentally as described in Examples 11, and 15 to 21 as described below.

In another embodiment, inputs to the model are organisms identified as significantly more abundant in COVID-19 patients with rapid viral clearance and recovery from disease than in those patients with prolonged disease or severe symptoms. Combinations of organisms with top scores for relative abundance and immune correlation are inputs to the model, simulating in silico the physical action of adding microbes to the gut of patients with severe viral disease. Classification is then performed using the machine learning model to estimate the probability that each augmented sample becomes that of a patient with rapid recovery. The hypothesis is that combinations of microbes that enable rapid recovery from viral infection according to the model are better candidates for therapeutic mixes. Each putative mix is scored by its mean predicted probability across all the augmented severe disease samples, with lower mean predicted severe disease probabilities corresponding to notionally better therapeutic candidates to improve viral clearance and lessen disease symptoms.

TABLE 9
List of exemplary live biotherapeutic compositions,
i.e., list of exemplary microbial combinations.

| Microbial | |
|---|---|
| Mix | Organism |
| 1 | Faecalibacterium prausnitzii |
| | Clostridium coccoides |
| | Ruminococcus gnavus |
| | Clostridium scindens |
| 2 | Faecalibacterium prausnitzii |
| | Clostridium coccoides |
| | Ruminococcus gnavus |
| | Clostridium scindens |
| | Akkermansia muciniphila |
| | Enterococcus hirae |
| 3 | Eggerthella lenta |
| | Gordonibacter urolithinfaciens |
| 4 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| 5 | Faecalibacterium prausnitzii |
| | Clostridium coccoides |
| | Ruminococcus gnavus |
| | Clostridium scindens |
| | Bacteroides thetaiotamicron |
| | Bacteroides caccae |
| | Gemmiger formicilis |
| 6 | Faecalibacterium prausnitzii |
| | Clostridium coccoides |
| | Ruminococcus gnavus |
| | Clostridium scindens |
| | Alistipes indistinctus |
| | Dorea formicigenerans |
| 7 | Faecalibacterium prausnitzii |
| | Clostridium coccoides |
| | Ruminococcus gnavus |
| | Clostridium scindens |
| | Bifidobacterium longum |
| | Bifidobacterium breve |
| 8 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Adlercreutzia equolifaciens |
| 9 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Adlercreutzia equolifaciens |
| | Senegalimassilia anaerobia |
| 10 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Adlercreutzia equolifaciens |
| | Senegalimassilia anaerobia |
| | Ellagibacter isourolithinifaciens |
| 11 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Adlercreutzia equolifaciens |
| | Ellagibacter isourolithinifaciens |
| 12 | Eggerthella lenta |
| | Gordonibacter urolithinfaciens |
| | Adlercreutzia equolifaciens |
| | Senegalimassilia anaerobia |
| | Ellagibacter isourolithinifaciens |
| 13 | Eggerthella lenta |
| | Gordonibacter urolithinfaciens |
| | Adlercreutzia equolifaciens |
| | Senegalimassilia anaerobia |
| | Ellagibacter isourolithinifaciens |
| | Collinsella aerofaciens |
| 14 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Adlercreutzia equolifaciens |
| | Senegalimassilia anaerobia |
| | Collinsella aerofaciens |
| 15 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Adlercreutzia equolifaciens |
| | Senegalimassilia anaerobia |
| | Collinsella aerofaciens |
| | Ellagibacter isourolithinifaciens |
| 16 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Ellagibacter isourolithinifaciens |
| 17 | Eggerthella lenta |
| | Gordonibacter urolithinfaciens |
| | Ellagibacter isourolithinifaciens |
| 18 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Paraeggerthella hongkongensis |
| 19 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Paraeggerthella hongkongensis |
| | Slackia isoflavoniconvertens |
| | Slackia equolifaciens |
| 20 | Faecalibacterium prausnitzii |
| | Clostridium coccoides |
| | Ruminococcus gnavus |
| | Clostridium scindens |
| | Gordonibacter urolithinfaciens |
| 21 | Eubacterium hallii |
| 22 | Faecalibacterium prausnitzii |
| | Clostridium coccoides |
| | Ruminococcus gnavus |
| | Clostridium scindens |
| | Eubacterium hallii |
| 23 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Eubacterium hallii |
| 24 | Faecalibacterium prausnitzii |
| | Clostridium coccoides |
| | Ruminococcus gnavus |
| | Clostridium scindens |
| | Akkermansia muciniphila |
| | Enterococcus hirae |
| | Eubacterium hallii |
| 25 | Blautia massiliensis |
| 26 | Faecalibacterium prausnitzii |
| | Clostridium coccoides |
| | Ruminococcus gnavus |
| | Clostridium scindens |
| | Blautia massiliensis |
| 27 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Blautia massiliensis |
| 28 | Faecalibacterium prausnitzii |
| | Clostridium coccoides |
| | Ruminococcus gnavus |
| | Clostridium scindens |
| | Akkermansia muciniphila |
| | Enterococcus hirae |
| | Blautia massiliensis |
| 29 | Faecalibacterium prausnitzii |
| | Clostridium coccoides |
| | Ruminococcus gnavus |
| | Clostridium scindens |
| | Eggerthella lenta |
| | Gordonibacter urolithinfaciens |
| | Blautia massiliensis |
| | Eubacterium hallii |
| 30 | Faecalibacterium prausnitzii |
| | Clostridium coccoides |
| | Ruminococcus gnavus |
| | Clostridium scindens |
| | Akkermansia muciniphila |
| | Enterococcus hirae |
| | Blautia massiliensis |
| | Eubacterium hallii |
| 31 | Faecalibacterium prausnitzii |
| | Clostridium coccoides |
| | Ruminococcus gnavus |
| | Clostridium scindens |
| | Gordonibacter urolithinfaciens |
| | Eubacterium hallii |
| 32 | Faecalibacterium prausnitzii |
| | Clostridium coccoides |
| | Ruminococcus gnavus |
| | Clostridium scindens |
| | Gordonibacter urolithinfaciens |
| | Eubacterium hallii |
| | Blautia massiliensis |
| 33 | Akkermansia muciniphila |
| | Faecalibacterium prausnitzii |
| 34 | Eubacterium Hallii |
| | Dorea Longicatena |
| | Blautia sp. SG-772 |
| 35 | Akkermansia muciniphila |
| | Faecalibacterium prausnitzii |
| | Eubacterium Hallii |
| | Dorea Longicatena |
| | Blautia sp. SG-772 |
| 36 | Akkermansia muciniphila |
| | Faecalibacterium prausnitzii |
| | Ruminococcus gnavus |
| 37 | Dorea Longicatena |
| | Dorea formicigenerans |
| | Blautia sp. SG-772 |
| | Eubacterium Hallii |
| | Ruminococcus faecis |
| | Coprococcus comes |
| 38 | Faecalibacterium prausnitzii |
| | Ruminococcus gnavus |
| 39 | Ruminococcus gnavus |
| | Eubacterium ramulus |
| | Gemmiger formicilis |
| 40 | Anaerostipes hadrus |
| | Dorea formicigenerans |
| | Dorea longicatena |
| | Coprococcus comes |
| | Ruminococcus faecis |
| 41 | Anaerostipes hadrus |
| | Dorea formicigenerans |
| | Dorea longicatena |
| | Coprococcus comes |
| | Ruminococcus faecis |
| | Ruminococcus gnavus |
| 42 | Anaerostipes hadrus |
| | Dorea formicigenerans |
| | Dorea longicatena |
| | Coprococcus comes |
| | Ruminococcus faecis |
| | Akkermansia muciniphila |
| 43 | Akkermansia muciniphila |
| | Eubacterium ramulus |
| | Gemmiger formicilis |
| 44 | Akkermansia muciniphila |
| | Ruminococcus gnavus |
| | Ruminococcus torques |
| | Bifidobacterium bifidum |
| 45 | Akkermansia muciniphila |
| | Ruminococcus gnavus |
| | Ruminococcus torques |
| 46 | Akkermansia muciniphila |
| | Ruminococcus torques |
| | Dorea longicatena |
| | Coprococcus comes |
| | Anaerostipes hadrus |
| 47 | Akkermansia muciniphila |
| | Roseburia inulivorans |
| | Dorea longicatena |
| | Coprococcus comes |
| | Anaerostipes hadrus |
| 48 | Dorea longicatena |
| | Coprococcus comes |
| | Anaerostipes hadrus |
| | Eubacterium Hallii |
| | Faecalibacterium prausnitzii |
| | Collinsella aerofaciens |
| 49 | Dorea longicatena |
| | Coprococcus comes |
| | Anaerostipes hadrus |
| | Eubacterium Hallii |
| | Faecalibacterium prausnitzii |
| | Blautia obeum |
| 50 | Akkermansia muciniphila |
| | Ruminococcus gnavus |
| | Dorea longicatena |
| | Coprococcus comes |
| | Anaerostipes hadrus |
| 51 | Akkermansia muciniphila |
| | Gemmiger formicilis |
| | Asacharobacter celatus |
| | Collinsella aerofaciens |
| | Alistipes putredinis |
| | Gordonibacter urolithinfaciens |
| 52 | Akkermansia muciniphila |
| | Monoglobus pectinilyticus |
| | Bacteroides galacturonicus |
| | Collinsella aerofaciens |
| | Ruminococcus gnavus |
| | Dorea longicatena |
| 53 | Akkermansia muciniphila |
| | Monoglobus pectinilyticus |
| | Bacteroides galacturonicus |
| | Collinsella aerofaciens |
| | Ruminococcus torques |
| | Dorea longicatena |
| 54 | Bifidobacterium bifidum C0005 |
| | Clostridium sp. AF36-4 C2893 |
| | Dorea sp. AM58-8 C2913 |
| | Erysipelotrichaceae bacterium GAM147 C2844 |
| 55 | Bifidobacterium bifidum C0005 |
| | Dorea sp. AM58-8 C2913 |
| | Erysipelotrichaceae bacterium GAM147 C2844 |
| | Ruminococcus lactaris C2149 |
| 56 | Bifidobacterium bifidum C0005 |
| | Dorea sp. AM58-8 C2913 |
| | Erysipelotrichaceae bacterium GAM147 C2844 |
| | Firmicutes bacterium AF12-30 C2644 |
| 57 | Bifidobacterium bifidum C0005 |
| | Dorea sp. AM58-8 C2913 |
| | Erysipelotrichaceae bacterium GAM147 C2844 |
| | Ruminococcus sp. OF03-6AA C2904 |
| 58 | Bifidobacterium bifidum C0005 |
| | Dorea longicatena C2131 |
| | Dorea sp. AM58-8 C2913 |
| | Erysipelotrichaceae bacterium GAM147 C2844 |
| 59 | Bifidobacterium bifidum C0005 |
| | Blautia obeum C2129 |
| | Dorea sp. AM58-8 C2913 |
| | Erysipelotrichaceae bacterium GAM147 C2844 |
| 60 | Bifidobacterium bifidum C0005 |
| | Coprococcus comes C2152 |
| | Dorea sp. AM58-8 C2913 |
| | Erysipelotrichaceae bacterium GAM147 C2844 |
| 61 | Clostridium sp. AF36-4 C2893 |
| | Dorea sp. AM58-8 C2913 |
| | Erysipelotrichaceae bacterium GAM147 C2844 |
| | Ruminococcus lactaris C2149 |
| 62 | Clostridium sp. AF36-4 C2893 |
| | Dorea sp. AM58-8 C2913 |
| | Erysipelotrichaceae bacterium GAM147 C2844 |
| | Firmicutes bacterium AF12-30 C2644 |
| 63 | Clostridium sp. AF36-4 C2893 |
| | Dorea sp. AM58-8 C2913 |
| | Erysipelotrichaceae bacterium GAM147 C2844 |
| | Ruminococcus sp. OF03-6AA C2904 |
| 64 | Clostridium sp. AF36-4 C2893 |
| | Dorea longicatena C2131 |
| | Dorea sp. AM58-8 C2913 |
| | Erysipelotrichaceae bacterium GAM147 C2844 |
| 65 | Blautia obeum C2129 |
| | Clostridium sp. AF36-4 C2893 |
| | Dorea sp. AM58-8 C2913 |
| | Erysipelotrichaceae bacterium GAM147 C2844 |
| 66 | Clostridium sp. AF36-4 C2893 |
| | Coprococcus comes C2152 |
| | Dorea sp. AM58-8 C2913 |
| | Erysipelotrichaceae bacterium GAM147 C2844 |
| 67 | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 68 | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 69 | Dorea longicatena C2131 |
| | Dorea sp. AM58-8 C2913 |
| | Erysipelotrichaceae bacterium GAM147 C2844 |
| | Ruminococcus lactaris C2149 |
| 70 | Blautia obeum C2129 |
| | Dorea sp. AM58-8 C2913 |
| | Erysipelotrichaceae bacterium GAM147 C2844 |
| | Ruminococcus lactaris C2149 |
| 71 | Coprococcus comes C2152 |
| | Dorea sp. AM58-8 C2913 |
| | Erysipelotrichaceae bacterium GAM147 C2844 |
| | Ruminococcus lactaris C2149 |
| 72 | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 73 | Dorea longicatena C2131 |
| | Dorea sp. AM58-8 C2913 |
| | Erysipelotrichaceae bacterium GAM147 C2844 |
| | Firmicutes bacterium AF12-30 C2644 |
| 74 | Blautia obeum C2129 |
| | Dorea sp. AM58-8 C2913 |
| | Erysipelotrichaceae bacterium GAM147 C2844 |
| | Firmicutes bacterium AF12-30 C2644 |
| 75 | Coprococcus comes C2152 |
| | Dorea sp. AM58-8 C2913 |
| | Erysipelotrichaceae bacterium GAM147 C2844 |
| | Firmicutes bacterium AF12-30 C2644 |
| 76 | Dorea longicatena C2131 |
| | Dorea sp. AM58-8 C2913 |
| | Erysipelotrichaceae bacterium GAM147 C2844 |
| | Ruminococcus sp. OF03-6AA C2904 |
| 77 | Blautia obeum C2129 |
| | Dorea sp. AM58-8 C2913 |
| | Erysipelotrichaceae bacterium GAM147 C2844 |
| | Ruminococcus sp. OF03-6AA C2904 |
| 78 | Coprococcus comes C2152 |
| | Dorea sp. AM58-8 C2913 |
| | Erysipelotrichaceae bacterium GAM147 C2844 |
| | Ruminococcus sp. OF03-6AA C2904 |
| 79 | Blautia obeum C2129 |
| | Dorea longicatena C2131 |
| | Dorea sp. AM58-8 C2913 |
| | Erysipelotrichaceae bacterium GAM147 C2844 |
| 80 | Coprococcus comes C2152 |
| | Dorea longicatena C2131 |
| | Dorea sp. AM58-8 C2913 |
| | Erysipelotrichaceae bacterium GAM147 C2844 |
| 81 | Blautia obeum C2129 |
| | Coprococcus comes C2152 |
| | Dorea sp. AM58-8 C2913 |
| | Erysipelotrichaceae bacterium GAM147 C2844 |
| 82 | Bifidobacterium bifidum C0005 |
| | Clostridium sp. AF36-4 C2893 |
| | Erysipelotrichaceae bacterium GAM147 C2844 |
| | Ruminococcus lactaris C2149 |
| 83 | Bifidobacterium bifidum C0005 |
| | Clostridium sp. AF36-4 C2893 |
| | Erysipelotrichaceae bacterium GAM147 C2844 |
| | Firmicutes bacterium AF12-30 C2644 |
| 84 | Bifidobacterium bifidum C0005 |
| | Clostridium sp. AF36-4 C2893 |
| | Erysipelotrichaceae bacterium GAM147 C2844 |
| | Ruminococcus sp. OF03-6AA C2904 |
| 85 | Bifidobacterium bifidum C0005 |
| | Clostridium sp. AF36-4 C2893 |
| | Dorea longicatena C2131 |
| | Erysipelotrichaceae bacterium GAM147 C2844 |
| 86 | Bifidobacterium bifidum C0005 |
| | Blautia obeum C2129 |
| | Clostridium sp. AF36-4 C2893 |
| | Erysipelotrichaceae bacterium GAM147 C2844 |
| 87 | Bifidobacterium bifidum C0005 |
| | Clostridium sp. AF36-4 C2893 |
| | Coprococcus comes C2152 |
| | Erysipelotrichaceae bacterium GAM147 C2844 |
| 88 | Bifidobacterium bifidum C0005 |
| | Erysipelotrichaceae bacterium GAM147 C2844 |
| | Firmicutes bacterium AF12-30 C2644 |
| | Ruminococcus lactaris C2149 |
| 89 | Bifidobacterium bifidum C0005 |
| | Erysipelotrichaceae bacterium GAM147 C2844 |
| | Ruminococcus lactaris C2149 |
| | Ruminococcus sp. OF03-6AA C2904 |
| 90 | Bifidobacterium bifidum C0005 |
| | Dorea longicatena C2131 |
| | Erysipelotrichaceae bacterium GAM147 C2844 |
| | Ruminococcus lactaris C2149 |
| 91 | Bifidobacterium bifidum C0005 |
| | Blautia obeum C2129 |
| | Erysipelotrichaceae bacterium GAM147 C2844 |
| | Ruminococcus lactaris C2149 |
| 92 | Bifidobacterium bifidum C0005 |
| | Coprococcus comes C2152 |
| | Erysipelotrichaceae bacterium GAM147 C2844 |
| | Ruminococcus lactaris C2149 |
| 93 | Bifidobacterium bifidum C0005 |
| | Erysipelotrichaceae bacterium GAM147 C2844 |
| | Firmicutes bacterium AF12-30 C2644 |
| | Ruminococcus sp. OF03-6AA C2904 |
| 94 | Bifidobacterium bifidum C0005 |
| | Dorea longicatena C2131 |
| | Erysipelotrichaceae bacterium GAM147 C2844 |
| | Firmicutes bacterium AF12-30 C2644 |
| 95 | Bifidobacterium bifidum C0005 |
| | Blautia obeum C2129 |
| | Erysipelotrichaceae bacterium GAM147 C2844 |
| | Firmicutes bacterium AF12-30 C2644 |
| 96 | Bifidobacterium bifidum C0005 |
| | Coprococcus comes C2152 |
| | Erysipelotrichaceae bacterium GAM147 C2844 |
| | Firmicutes bacterium AF12-30 C2644 |
| 97 | Bifidobacterium bifidum C0005 |
| | Dorea longicatena C2131 |
| | Erysipelotrichaceae bacterium GAM147 C2844 |
| | Ruminococcus sp. OF03-6AA C2904 |
| 98 | Bifidobacterium bifidum C0005 |
| | Blautia obeum C2129 |
| | Erysipelotrichaceae bacterium GAM147 C2844 |
| | Ruminococcus sp. OF03-6AA C2904 |
| 99 | Bifidobacterium bifidum C0005 |
| | Coprococcus comes C2152 |
| | Erysipelotrichaceae bacterium GAM147 C2844 |
| | Ruminococcus sp. OF03-6AA C2904 |
| 100 | Bifidobacterium bifidum C0005 |
| | Blautia obeum C2129 |
| | Dorea longicatena C2131 |
| | Erysipelotrichaceae bacterium GAM147 C2844 |
| 101 | Bifidobacterium bifidum C0005 |
| | Coprococcus comes C2152 |
| | Dorea longicatena C2131 |
| | Erysipelotrichaceae bacterium GAM147 C2844 |
| 102 | Bifidobacterium bifidum C0005 |
| | Blautia obeum C2129 |
| | Coprococcus comes C2152 |
| | Erysipelotrichaceae bacterium GAM147 C2844 |
| 103 | Clostridium sp. AF36-4 C2893 |
| | Erysipelotrichaceae bacterium GAM147 C2844 |
| | Firmicutes bacterium AF12-30 C2644 |
| | Ruminococcus lactaris C2149 |
| 104 | Clostridium sp. AF36-4 C2893 |
| | Erysipelotrichaceae bacterium GAM147 C2844 |
| | Ruminococcus lactaris C2149 |
| | Ruminococcus sp. OF03-6AA C2904 |
| 105 | Clostridium sp. AF36-4 C2893 |
| | Dorea longicatena C2131 |
| | Erysipelotrichaceae bacterium GAM147 C2844 |
| | Ruminococcus lactaris C2149 |
| 106 | Blautia obeum C2129 |
| | Clostridium sp. AF36-4 C2893 |
| | Erysipelotrichaceae bacterium GAM147 C2844 |
| | Ruminococcus lactaris C2149 |
| 107 | Clostridium sp. AF36-4 C2893 |
| | Coprococcus comes C2152 |
| | Erysipelotrichaceae bacterium GAM147 C2844 |
| | Ruminococcus lactaris C2149 |
| 108 | Clostridium sp. AF36-4 C2893 |
| | Erysipelotrichaceae bacterium GAM147 C2844 |
| | Firmicutes bacterium AF12-30 C2644 |
| | Ruminococcus sp. OF03-6AA C2904 |
| 109 | Clostridium sp. AF36-4 C2893 |
| | Dorea longicatena C2131 |
| | Erysipelotrichaceae bacterium GAM147 C2844 |
| | Firmicutes bacterium AF12-30 C2644 |
| 110 | Blautia obeum C2129 |
| | Clostridium sp. AF36-4 C2893 |
| | Erysipelotrichaceae bacterium GAM147 C2844 |
| | Firmicutes bacterium AF12-30 C2644 |
| 111 | Clostridium sp. AF36-4 C2893 |
| | Coprococcus comes C2152 |
| | Erysipelotrichaceae bacterium GAM147 C2844 |
| | Firmicutes bacterium AF12-30 C2644 |
| 112 | Clostridium sp. AF36-4 C2893 |
| | Dorea longicatena C2131 |
| | Erysipelotrichaceae bacterium GAM147 C2844 |
| | Ruminococcus sp. OF03-6AA C2904 |
| 113 | Blautia obeum C2129 |
| | Clostridium sp. AF36-4 C2893 |
| | Erysipelotrichaceae bacterium GAM147 C2844 |
| | Ruminococcus sp. OF03-6AA C2904 |
| 114 | Clostridium sp. AF36-4 C2893 |
| | Coprococcus comes C2152 |
| | Erysipelotrichaceae bacterium GAM147 C2844 |
| | Ruminococcus sp. OF03-6AA C2904 |
| 115 | Blautia obeum C2129 |
| | Clostridium sp. AF36-4 C2893 |
| | Dorea longicatena C2131 |
| | Erysipelotrichaceae bacterium GAM147 C2844 |
| 116 | Clostridium sp. AF36-4 C2893 |
| | Coprococcus comes C2152 |
| | Dorea longicatena C2131 |
| | Erysipelotrichaceae bacterium GAM147 C2844 |
| 117 | Blautia obeum C2129 |
| | Clostridium sp. AF36-4 C2893 |
| | Coprococcus comes C2152 |
| | Erysipelotrichaceae bacterium GAM147 C2844 |
| 118 | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 119 | Dorea longicatena C2131 |
| | Erysipelotrichaceae bacterium GAM 147 C2844 |
| | Firmicutes bacterium AF12-30 C2644 |
| | Ruminococcus lactaris C2149 |
| 120 | Blautia obeum C2129 |
| | Erysipelotrichaceae bacterium GAM147 C2844 |
| | Firmicutes bacterium AF12-30 C2644 |
| | Ruminococcus lactaris C2149 |
| 121 | Coprococcus comes C2152 |
| | Erysipelotrichaceae bacterium GAM147 C2844 |
| | Firmicutes bacterium AF12-30 C2644 |
| | Ruminococcus lactaris C2149 |
| 122 | Dorea longicatena C2131 |
| | Erysipelotrichaceae bacterium GAM147 C2844 |
| | Ruminococcus lactaris C2149 |
| | Ruminococcus sp. OF03-6AA C2904 |
| 123 | Blautia obeum C2129 |
| | Erysipelotrichaceae bacterium GAM147 C2844 |
| | Ruminococcus lactaris C2149 |
| | Ruminococcus sp. OF03-6AA C2904 |
| 124 | Coprococcus comes C2152 |
| | Erysipelotrichaceae bacterium GAM147 C2844 |
| | Ruminococcus lactaris C2149 |
| | Ruminococcus sp. OF03-6AA C2904 |
| 125 | Blautia obeum C2129 |
| | Dorea longicatena C2131 |
| | Erysipelotrichaceae bacterium GAM147 C2844 |
| | Ruminococcus lactaris C2149 |
| 126 | Coprococcus comes C2152 |
| | Dorea longicatena C2131 |
| | Erysipelotrichaceae bacterium GAM147 C2844 |
| | Ruminococcus lactaris C2149 |
| 127 | Blautia obeum C2129 |
| | Coprococcus comes C2152 |
| | Erysipelotrichaceae bacterium GAM147 C2844 |
| | Ruminococcus lactaris C2149 |
| 128 | Dorea longicatena C2131 |
| | Erysipelotrichaceae bacterium GAM147 C2844 |
| | Firmicutes bacterium AF12-30 C2644 |
| | Ruminococcus sp. OF03-6AA C2904 |
| 129 | Blautia obeum C2129 |
| | Erysipelotrichaceae bacterium GAM147 C2844 |
| | Firmicutes bacterium AF12-30 C2644 |
| | Ruminococcus sp. OF03-6AA C2904 |
| 130 | Coprococcus comes C2152 |
| | Erysipelotrichaceae bacterium GAM147 C2844 |
| | Firmicutes bacterium AF12-30 C2644 |
| | Ruminococcus sp. OF03-6AA C2904 |
| 131 | Blautia obeum C2129 |
| | Dorea longicatena C2131 |
| | Erysipelotrichaceae bacterium GAM147 C2844 |
| | Firmicutes bacterium AF12-30 C2644 |
| 132 | Coprococcus comes C2152 |
| | Dorea longicatena C2131 |
| | Erysipelotrichaceae bacterium GAM147 C2844 |
| | Firmicutes bacterium AF12-30 C2644 |
| 133 | Blautia obeum C2129 |
| | Coprococcus comes C2152 |
| | Erysipelotrichaceae bacterium GAM147 C2844 |
| | Firmicutes bacterium AF12-30 C2644 |
| 134 | Blautia obeum C2129 |
| | Dorea longicatena C2131 |
| | Erysipelotrichaceae bacterium GAM147 C2844 |
| | Ruminococcus sp. OF03-6AA C2904 |
| 135 | Coprococcus comes C2152 |
| | Dorea longicatena C2131 |
| | Erysipelotrichaceae bacterium GAM147 C2844 |
| | Ruminococcus sp. OF03-6AA C2904 |
| 136 | Blautia obeum C2129 |
| | Coprococcus comes C2152 |
| | Erysipelotrichaceae bacterium GAM147 C2844 |
| | Ruminococcus sp. OF03-6AA C2904 |
| 137 | Blautia obeum C2129 |
| | Coprococcus comes C2152 |
| | Dorea longicatena C2131 |
| | Erysipelotrichaceae bacterium GAM147 C2844 |
| 138 | Bifidobacterium bifidum C0005 |
| | Clostridium sp. AF36-4 C2893 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 139 | Bifidobacterium bifidum C0005 |
| | Clostridium sp. AF36-4 C2893 |
| | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 140 | Bifidobacterium bifidum C0005 |
| | Clostridium sp. AF36-4 C2893 |
| | Dorea longicatena C2131 |
| | Dorea sp. AM58-8 C2913 |
| | Erysipelotrichaceae bacterium GAM147 C2844 |
| | Ruminococcus lactaris C2149 |
| 141 | Bifidobacterium bifidum C0005 |
| | Blautia obeum C2129 |
| | Clostridium sp. AF36-4 C2893 |
| | Dorea sp. AM58-8 C2913 |
| | Erysipelotrichaceae bacterium GAM147 C2844 |
| | Ruminococcus lactaris C2149 |
| 142 | Bifidobacterium bifidum C0005 |
| | Clostridium sp. AF36-4 C2893 |
| | Coprococcus comes C2152 |
| | Dorea sp. AM58-8 C2913 |
| | Erysipelotrichaceae bacterium GAM147 C2844 |
| | Ruminococcus lactaris C2149 |
| 143 | Bifidobacterium bifidum C0005 |
| | Clostridium sp. AF36-4 C2893 |
| | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 144 | Bifidobacterium bifidum C0005 |
| | Clostridium sp. AF36-4 C2893 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| 145 | Bifidobacterium bifidum C0005 |
| | Blautia obeum C2129 |
| | Clostridium sp. AF36-4 C2893 |
| | Dorea sp. AM58-8 C2913 |
| | Erysipelotrichaceae bacterium GAM147 C2844 |
| | Firmicutes bacterium AF12-30 C2644 |
| 146 | Bifidobacterium bifidum C0005 |
| | Clostridium sp. AF36-4 C2893 |
| | Coprococcus comes C2152 |
| | Dorea sp. AM58-8 C2913 |
| | Erysipelotrichaceae bacterium GAM147 C2844 |
| | Firmicutes bacterium AF12-30 C2644 |
| 147 | Bifidobacterium bifidum C0005 |
| | Clostridium sp. AF36-4 C2893 |
| | Dorea longicatena C2131 |
| | Dorea sp. AM58-8 C2913 |
| | Erysipelotrichaceae bacterium GAM147 C2844 |
| | Ruminococcus sp. OF03-6AA C2904 |
| 148 | Bifidobacterium bifidum C0005 |
| | Blautia obeum C2129 |
| | Clostridium sp. AF36-4 C2893 |
| | Dorea sp. AM58-8 C2913 |
| | Erysipelotrichaceae bacterium GAM 147 C2844 |
| | Ruminococcus sp. OF03-6AA C2904 |
| 149 | Bifidobacterium bifidum C0005 |
| | Clostridium sp. AF36-4 C2893 |
| | Coprococcus comes C2152 |
| | Dorea sp. AM58-8 C2913 |
| | Erysipelotrichaceae bacterium GAM147 C2844 |
| | Ruminococcus sp. OF03-6AA C2904 |
| 150 | Bifidobacterium bifidum C0005 |
| | Blautia obeum C2129 |
| | Clostridium sp. AF36-4 C2893 |
| | Dorea longicatena C2131 |
| | Dorea sp. AM58-8 C2913 |
| | Erysipelotrichaceae bacterium GAM147 C2844 |
| 151 | Bifidobacterium bifidum C0005 |
| | Clostridium sp. AF36-4 C2893 |
| | Coprococcus comes C2152 |
| | Dorea longicatena C2131 |
| | Dorea sp. AM58-8 C2913 |
| | Erysipelotrichaceae bacterium GAM147 C2844 |
| 152 | Bifidobacterium bifidum C0005 |
| | Blautia obeum C2129 |
| | Clostridium sp. AF36-4 C2893 |
| | Coprococcus comes C2152 |
| | Dorea sp. AM58-8 C2913 |
| | Erysipelotrichaceae bacterium GAM147 C2844 |
| 153 | Bifidobacterium bifidum C0005 |
| | Dorea sp. AM58-8 C2913 |
| | Erysipelotrichaceae bacterium GAM147 |
| | C2844 |
| | Firmicutes bacterium AF12-30 C2644 |
| | Ruminococcus lactaris C2149 |
| | Ruminococcus sp. OF03-6AA C2904 |
| 154 | Bifidobacterium bifidum C0005 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 155 | Bifidobacterium bifidum C0005 |
| | Blautia obeum C2129 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 156 | Bifidobacterium bifidum C0005 |
| | Coprococcus comes C2152 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 157 | Bifidobacterium bifidum C0005 |
| | Dorea longicatena C2131 |
| | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 158 | Bifidobacterium bifidum C0005 |
| | Blautia obeum C2129 |
| | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 159 | Bifidobacterium bifidum C0005 |
| | Coprococcus comes C2152 |
| | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 160 | Bifidobacterium bifidum C0005 |
| | Blautia obeum C2129 |
| | Dorea longicatena C2131 |
| | Dorea sp. AM58-8 C2913 |
| | Erysipelotrichaceae bacterium GAM147 C2844 |
| | Ruminococcus lactaris C2149 |
| 161 | Bifidobacterium bifidum C0005 |
| | Coprococcus comes C2152 |
| | Dorea longicatena C2131 |
| | Dorea sp. AM58-8 C2913 |
| | Erysipelotrichaceae bacterium GAM147 C2844 |
| | Ruminococcus lactaris C2149 |
| 162 | Bifidobacterium bifidum C0005 |
| | Blautia obeum C2129 |
| | Coprococcus comes C2152 |
| | Dorea sp. AM58-8 C2913 |
| | Erysipelotrichaceae bacterium GAM147 C2844 |
| | Ruminococcus lactaris C2149 |
| 163 | Bifidobacterium bifidum C0005 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 164 | Bifidobacterium bifidum C0005 |
| | Blautia obeum C2129 |
| | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 165 | Bifidobacterium bifidum C0005 |
| | Coprococcus comes C2152 |
| | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 166 | Bifidobacterium bifidum C0005 |
| | Blautia obeum C2129 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| 167 | Bifidobacterium bifidum C0005 |
| | Coprococcus comes C2152 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| 168 | Bifidobacterium bifidum C0005 |
| | Blautia obeum C2129 |
| | Coprococcus comes C2152 |
| | Dorea sp. AM58-8 C2913 |
| | Erysipelotrichaceae bacterium GAM147 C2844 |
| | Firmicutes bacterium AF12-30 C2644 |
| 169 | Bifidobacterium bifidum C0005 |
| | Blautia obeum C2129 |
| | Dorea longicatena C2131 |
| | Dorea sp. AM58-8 C2913 |
| | Erysipelotrichaceae bacterium GAM147 C2844 |
| | Ruminococcus sp. OF03-6AA C2904 |
| 170 | Bifidobacterium bifidum C0005 |
| | Coprococcus comes C2152 |
| | Dorea longicatena C2131 |
| | Dorea sp. AM58-8 C2913 |
| | Erysipelotrichaceae bacterium GAM147 C2844 |
| | Ruminococcus sp. OF03-6AA C2904 |
| 171 | Bifidobacterium bifidum C0005 |
| | Blautia obeum C2129 |
| | Coprococcus comes C2152 |
| | Dorea sp. AM58-8 C2913 |
| | Erysipelotrichaceae bacterium GAM147 C2844 |
| | Ruminococcus sp. OF03-6AA C2904 |
| 172 | Bifidobacterium bifidum C0005 |
| | Blautia obeum C2129 |
| | Coprococcus comes C2152 |
| | Dorea longicatena C2131 |
| | Dorea sp. AM58-8 C2913 |
| | Erysipelotrichaceae bacterium GAM147 C2844 |
| 173 | Clostridium sp. AF36-4 C2893 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 174 | Clostridium sp. AF36-4 C2893 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 175 | Blautia obeum C2129 |
| | Clostridium sp. AF36-4 C2893 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 176 | Clostridium sp. AF36-4 C2893 |
| | Coprococcus comes C2152 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 177 | Clostridium sp. AF36-4 C2893 |
| | Dorea longicatena C2131 |
| | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 178 | Blautia obeum C2129 |
| | Clostridium sp. AF36-4 C2893 |
| | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 179 | Clostridium sp. AF36-4 C2893 |
| | Coprococcus comes C2152 |
| | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 180 | Blautia obeum C2129 |
| | Clostridium sp. AF36-4 C2893 |
| | Dorea longicatena C2131 |
| | Dorea sp. AM58-8 C2913 |
| | Erysipelotrichaceae bacterium GAM147 C2844 |
| | Ruminococcus lactaris C2149 |
| 181 | Clostridium sp. AF36-4 C2893 |
| | Coprococcus comes C2152 |
| | Dorea longicatena C2131 |
| | Dorea sp. AM58-8 C2913 |
| | Erysipelotrichaceae bacterium GAM147 C2844 |
| | Ruminococcus lactaris C2149 |
| 182 | Blautia obeum C2129 |
| | Clostridium sp. AF36-4 C2893 |
| | Coprococcus comes C2152 |
| | Dorea sp. AM58-8 C2913 |
| | Erysipelotrichaceae bacterium GAM147 C2844 |
| | Ruminococcus lactaris C2149 |
| 183 | Clostridium sp. AF36-4 C2893 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 184 | Blautia obeum C2129 |
| | Clostridium sp. AF36-4 C2893 |
| | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 185 | Clostridium sp. AF36-4 C2893 |
| | Coprococcus comes C2152 |
| | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 186 | Blautia obeum C2129 |
| | Clostridium sp. AF36-4 C2893 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| 187 | Clostridium sp. AF36-4 C2893 |
| | Coprococcus comes C2152 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| 188 | Blautia obeum C2129 |
| | Clostridium sp. AF36-4 C2893 |
| | Coprococcus comes C2152 |
| | Dorea sp. AM58-8 C2913 |
| | Erysipelotrichaceae bacterium GAM147 C2844 |
| | Firmicutes bacterium AF12-30 C2644 |
| 189 | Blautia obeum C2129 |
| | Clostridium sp. AF36-4 C2893 |
| | Dorea longicatena C2131 |
| | Dorea sp. AM58-8 C2913 |
| | Erysipelotrichaceae bacterium GAM147 C2844 |
| | Ruminococcus sp. OF03-6AA C2904 |
| 190 | Clostridium sp. AF36-4 C2893 |
| | Coprococcus comes C2152 |
| | Dorea longicatena C2131 |
| | Dorea sp. AM58-8 C2913 |
| | Erysipelotrichaceae bacterium GAM147 C2844 |
| | Ruminococcus sp. OF03-6AA C2904 |
| 191 | Blautia obeum C2129 |
| | Clostridium sp. AF36-4 C2893 |
| | Coprococcus comes C2152 |
| | Dorea sp. AM58-8 C2913 |
| | Erysipelotrichaceae bacterium GAM147 C2844 |
| | Ruminococcus sp. OF03-6AA C2904 |
| 192 | Blautia obeum C2129 |
| | Clostridium sp. AF36-4 C2893 |
| | Coprococcus comes C2152 |
| | Dorea longicatena C2131 |
| | Dorea sp. AM58-8 C2913 |
| | Erysipelotrichaceae bacterium GAM147 C2844 |
| 193 | Dorea longicatena C2131 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 194 | Blautia obeum C2129 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 195 | Coprococcus comes C2152 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 196 | Blautia obeum C2129 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 197 | Coprococcus comes C2152 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 198 | Blautia obeum C2129 |
| | Coprococcus comes C2152 |
| | Dorea sp. AM58-8 C2913 |
| | Erysipelotrichaceae bacterium GAM147 C2844 |
| | Firmicutes bacterium AF12-30 C2644 |
| | Ruminococcus lactaris C2149 |
| 199 | Blautia obeum C2129 |
| | Dorea longicatena C2131 |
| | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 200 | Coprococcus comes C2152 |
| | Dorea longicatena C2131 |
| | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 201 | Blautia obeum C2129 |
| | Coprococcus comes C2152 |
| | Dorea sp. AM58-8 C2913 |
| | Erysipelotrichaceae bacterium GAM147 C2844 |
| | Ruminococcus lactaris C2149 |
| | Ruminococcus sp. OF03-6AA C2904 |
| 202 | Blautia obeum C2129 |
| | Coprococcus comes C2152 |
| | Dorea longicatena C2131 |
| | Dorea sp. AM58-8 C2913 |
| | Erysipelotrichaceae bacterium GAM147 C2844 |
| | Ruminococcus lactaris C2149 |
| 203 | Blautia obeum C2129 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 204 | Coprococcus comes C2152 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 205 | Blautia obeum C2129 |
| | Coprococcus comes C2152 |
| | Dorea sp. AM58-8 C2913 |
| | Erysipelotrichaceae bacterium GAM147 C2844 |
| | Firmicutes bacterium AF12-30 C2644 |
| | Ruminococcus sp. OF03-6AA C2904 |
| 206 | Blautia obeum C2129 |
| | Coprococcus comes C2152 |
| | Dorea longicatena C2131 |
| | Dorea sp. AM58-8 C2913 |
| | Erysipelotrichaceae bacterium GAM147 C2844 |
| | Firmicutes bacterium AF12-30 C2644 |
| 207 | Blautia obeum C2129 |
| | Coprococcus comes C2152 |
| | Dorea longicatena C2131 |
| | Dorea sp. AM58-8 C2913 |
| | Erysipelotrichaceae bacterium GAM147 C2844 |
| | Ruminococcus sp. OF03-6AA C2904 |
| 208 | Bifidobacterium bifidum C0005 |
| | Clostridium sp. AF36-4 C2893 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 209 | Bifidobacterium bifidum C0005 |
| | Clostridium sp. AF36-4 C2893 |
| | Dorea longicatena C2131 |
| | Erysipelotrichaceae bacterium GAM147 C2844 |
| | Firmicutes bacterium AF12-30 C2644 |
| | Ruminococcus lactaris C2149 |
| 210 | Bifidobacterium bifidum C0005 |
| | Blautia obeum C2129 |
| | Clostridium sp. AF36-4 C2893 |
| | Erysipelotrichaceae bacterium GAM147 C2844 |
| | Firmicutes bacterium AF12-30 C2644 |
| | Ruminococcus lactaris C2149 |
| 211 | Bifidobacterium bifidum C0005 |
| | Clostridium sp. AF36-4 C2893 |
| | Coprococcus comes C2152 |
| | Erysipelotrichaceae bacterium GAM147 C2844 |
| | Firmicutes bacterium AF12-30 C2644 |
| | Ruminococcus lactaris C2149 |
| 212 | Bifidobacterium bifidum C0005 |
| | Clostridium sp. AF36-4 C2893 |
| | Dorea longicatena C2131 |
| | Erysipelotrichaceae bacterium GAM147 C2844 |
| | Ruminococcus lactaris C2149 |
| | Ruminococcus sp. OF03-6AA C2904 |
| 213 | Bifidobacterium bifidum C0005 |
| | Blautia obeum C2129 |
| | Clostridium sp. AF36-4 C2893 |
| | Erysipelotrichaceae bacterium GAM147 C2844 |
| | Ruminococcus lactaris C2149 |
| | Ruminococcus sp. OF03-6AA C2904 |
| 214 | Bifidobacterium bifidum C0005 |
| | Clostridium sp. AF36-4 C2893 |
| | Coprococcus comes C2152 |
| | Erysipelotrichaceae bacterium GAM147 C2844 |
| | Ruminococcus lactaris C2149 |
| | Ruminococcus sp. OF03-6AA C2904 |
| 215 | Bifidobacterium bifidum C0005 |
| | Blautia obeum C2129 |
| | Clostridium sp. AF36-4 C2893 |
| | Dorea longicatena C2131 |
| | Erysipelotrichaceae bacterium GAM147 C2844 |
| | Ruminococcus lactaris C2149 |
| 216 | Bifidobacterium bifidum C0005 |
| | Clostridium sp. AF36-4 C2893 |
| | Coprococcus comes C2152 |
| | Dorea longicatena C2131 |
| | Erysipelotrichaceae bacterium GAM147 C2844 |
| | Ruminococcus lactaris C2149 |
| 217 | Bifidobacterium bifidum C0005 |
| | Blautia obeum C2129 |
| | Clostridium sp. AF36-4 C2893 |
| | Coprococcus comes C2152 |
| | Erysipelotrichaceae bacterium GAM147 C2844 |
| | Ruminococcus lactaris C2149 |
| 218 | Bifidobacterium bifidum C0005 |
| | Clostridium sp. AF36-4 C2893 |
| | Dorea longicatena C2131 |
| | Erysipelotrichaceae bacterium GAM147 C2844 |
| | Firmicutes bacterium AF12-30 C2644 |
| | Ruminococcus sp. OF03-6AA C2904 |
| 219 | Bifidobacterium bifidum C0005 |
| | Blautia obeum C2129 |
| | Clostridium sp. AF36-4 C2893 |
| | Erysipelotrichaceae bacterium GAM147 C2844 |
| | Firmicutes bacterium AF12-30 C2644 |
| | Ruminococcus sp. OF03-6AA C2904 |
| 220 | Bifidobacterium bifidum C0005 |
| | Clostridium sp. AF36-4 C2893 |
| | Coprococcus comes C2152 |
| | Erysipelotrichaceae bacterium GAM147 C2844 |
| | Firmicutes bacterium AF12-30 C2644 |
| | Ruminococcus sp. OF03-6AA C2904 |
| 221 | Bifidobacterium bifidum C0005 |
| | Blautia obeum C2129 |
| | Clostridium sp. AF36-4 C2893 |
| | Dorea longicatena C2131 |
| | Erysipelotrichaceae bacterium GAM147 C2844 |
| | Firmicutes bacterium AF12-30 C2644 |
| 222 | Bifidobacterium bifidum C0005 |
| | Clostridium sp. AF36-4 C2893 |
| | Coprococcus comes C2152 |
| | Dorea longicatena C2131 |
| | Erysipelotrichaceae bacterium GAM147 C2844 |
| | Firmicutes bacterium AF12-30 C2644 |
| 223 | Bifidobacterium bifidum C0005 |
| | Blautia obeum C2129 |
| | Clostridium sp. AF36-4 C2893 |
| | Coprococcus comes C2152 |
| | Erysipelotrichaceae bacterium GAM147 C2844 |
| | Firmicutes bacterium AF12-30 C2644 |
| 224 | Bifidobacterium bifidum C0005 |
| | Blautia obeum C2129 |
| | Clostridium sp. AF36-4 C2893 |
| | Dorea longicatena C2131 |
| | Erysipelotrichaceae bacterium GAM147 C2844 |
| | Ruminococcus sp. OF03-6AA C2904 |
| 225 | Bifidobacterium bifidum C0005 |
| | Clostridium sp. AF36-4 C2893 |
| | Coprococcus comes C2152 |
| | Dorea longicatena C2131 |
| | Erysipelotrichaceae bacterium GAM147 C2844 |
| | Ruminococcus sp. OF03-6AA C2904 |
| 226 | Bifidobacterium bifidum C0005 |
| | Blautia obeum C2129 |
| | Clostridium sp. AF36-4 C2893 |
| | Coprococcus comes C2152 |
| | Erysipelotrichaceae bacterium GAM147 C2844 |
| | Ruminococcus sp. OF03-6AA C2904 |
| 227 | Bifidobacterium bifidum C0005 |
| | Blautia obeum C2129 |
| | Clostridium sp. AF36-4 C2893 |
| | Coprococcus comes C2152 |
| | Dorea longicatena C2131 |
| | Erysipelotrichaceae bacterium GAM147 C2844 |
| 228 | Bifidobacterium bifidum C0005 |
| | Dorea longicatena C2131 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 229 | Bifidobacterium bifidum C0005 |
| | Blautia obeum C2129 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 230 | Bifidobacterium bifidum C0005 |
| | Coprococcus comes C2152 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 231 | Bifidobacterium bifidum C0005 |
| | Blautia obeum C2129 |
| | Dorea longicatena C2131 |
| | Erysipelotrichaceae bacterium GAM147 C2844 |
| | Firmicutes bacterium AF12-30 C2644 |
| | Ruminococcus lactaris C2149 |
| 232 | Bifidobacterium bifidum C0005 |
| | Coprococcus comes C2152 |
| | Dorea longicatena C2131 |
| | Erysipelotrichaceae bacterium GAM147 C2844 |
| | Firmicutes bacterium AF12-30 C2644 |
| | Ruminococcus lactaris C2149 |
| 233 | Bifidobacterium bifidum C0005 |
| | Blautia obeum C2129 |
| | Coprococcus comes C2152 |
| | Erysipelotrichaceae bacterium GAM147 C2844 |
| | Firmicutes bacterium AF12-30 C2644 |
| | Ruminococcus lactaris C2149 |
| 234 | Bifidobacterium bifidum C0005 |
| | Blautia obeum C2129 |
| | Dorea longicatena C2131 |
| | Erysipelotrichaceae bacterium GAM147 C2844 |
| | Ruminococcus lactaris C2149 |
| | Ruminococcus sp. OF03-6AA C2904 |
| 235 | Bifidobacterium bifidum C0005 |
| | Coprococcus comes C2152 |
| | Dorea longicatena C2131 |
| | Erysipelotrichaceae bacterium GAM147 C2844 |
| | Ruminococcus lactaris C2149 |
| | Ruminococcus sp. OF03-6AA C2904 |
| 236 | Bifidobacterium bifidum C0005 |
| | Blautia obeum C2129 |
| | Coprococcus comes C2152 |
| | Erysipelotrichaceae bacterium GAM147 C2844 |
| | Ruminococcus lactaris C2149 |
| | Ruminococcus sp. OF03-6AA C2904 |
| 237 | Bifidobacterium bifidum C0005 |
| | Blautia obeum C2129 |
| | Coprococcus comes C2152 |
| | Dorea longicatena C2131 |
| | Erysipelotrichaceae bacterium GAM147 C2844 |
| | Ruminococcus lactaris C2149 |
| 238 | Bifidobacterium bifidum C0005 |
| | Blautia obeum C2129 |
| | Dorea longicatena C2131 |
| | Erysipelotrichaceae bacterium GAM147 C2844 |
| | Firmicutes bacterium AF12-30 C2644 |
| | Ruminococcus sp. OF03-6AA C2904 |
| 239 | Bifidobacterium bifidum C0005 |
| | Coprococcus comes C2152 |
| | Dorea longicatena C2131 |
| | Erysipelotrichaceae bacterium GAM147 C2844 |
| | Firmicutes bacterium AF12-30 C2644 |
| | Ruminococcus sp. OF03-6AA C2904 |
| 240 | Bifidobacterium bifidum C0005 |
| | Blautia obeum C2129 |
| | Coprococcus comes C2152 |
| | Erysipelotrichaceae bacterium GAM147 C2844 |
| | Firmicutes bacterium AF12-30 C2644 |
| | Ruminococcus sp. OF03-6AA C2904 |
| 241 | Bifidobacterium bifidum C0005 |
| | Blautia obeum C2129 |
| | Coprococcus comes C2152 |
| | Dorea longicatena C2131 |
| | Erysipelotrichaceae bacterium GAM147 C2844 |
| | Firmicutes bacterium AF12-30 C2644 |
| 242 | Bifidobacterium bifidum C0005 |
| | Blautia obeum C2129 |
| | Coprococcus comes C2152 |
| | Dorea longicatena C2131 |
| | Erysipelotrichaceae bacterium GAM147 C2844 |
| | Ruminococcus sp. OF03-6AA C2904 |
| 243 | Clostridium sp. AF36-4 C2893 |
| | Dorea longicatena C2131 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 244 | Blautia obeum C2129 |
| | Clostridium sp. AF36-4 C2893 |
| | Erysipelotrichaceae bacterium GAM 147 C2844 |
| | Firmicutes bacterium AF12-30 C2644 |
| | Ruminococcus lactaris C2149 |
| | Ruminococcus sp. OF03-6AA C2904 |
| 245 | Clostridium sp. AF36-4 C2893 |
| | Coprococcus comes C2152 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 246 | Blautia obeum C2129 |
| | Clostridium sp. AF36-4 C2893 |
| | Dorea longicatena C2131 |
| | Erysipelotrichaceae bacterium GAM147 C2844 |
| | Firmicutes bacterium AF12-30 C2644 |
| | Ruminococcus lactaris C2149 |
| 247 | Clostridium sp. AF36-4 C2893 |
| | Coprococcus comes C2152 |
| | Dorea longicatena C2131 |
| | Erysipelotrichaceae bacterium GAM147 C2844 |
| | Firmicutes bacterium AF12-30 C2644 |
| | Ruminococcus lactaris C2149 |
| 248 | Blautia obeum C2129 |
| | Clostridium sp. AF36-4 C2893 |
| | Coprococcus comes C2152 |
| | Erysipelotrichaceae bacterium GAM147 C2844 |
| | Firmicutes bacterium AF12-30 C2644 |
| | Ruminococcus lactaris C2149 |
| 249 | Blautia obeum C2129 |
| | Clostridium sp. AF36-4 C2893 |
| | Dorea longicatena C2131 |
| | Erysipelotrichaceae bacterium GAM147 C2844 |
| | Ruminococcus lactaris C2149 |
| | Ruminococcus sp. OF03-6AA C2904 |
| 250 | Clostridium sp. AF36-4 C2893 |
| | Coprococcus comes C2152 |
| | Dorea longicatena C2131 |
| | Erysipelotrichaceae bacterium GAM147 C2844 |
| | Ruminococcus lactaris C2149 |
| | Ruminococcus sp. OF03-6AA C2904 |
| 251 | Blautia obeum C2129 |
| | Clostridium sp. AF36-4 C2893 |
| | Coprococcus comes C2152 |
| | Erysipelotrichaceae bacterium GAM147 C2844 |
| | Ruminococcus lactaris C2149 |
| | Ruminococcus sp. OF03-6AA C2904 |
| 252 | Blautia obeum C2129 |
| | Clostridium sp. AF36-4 C2893 |
| | Coprococcus comes C2152 |
| | Dorea longicatena C2131 |
| | Erysipelotrichaceae bacterium GAM147 C2844 |
| | Ruminococcus lactaris C2149 |
| 253 | Blautia obeum C2129 |
| | Clostridium sp. AF36-4 C2893 |
| | Dorea longicatena C2131 |
| | Erysipelotrichaceae bacterium GAM147 C2844 |
| | Firmicutes bacterium AF12-30 C2644 |
| | Ruminococcus sp. OF03-6AA C2904 |
| 254 | Clostridium sp. AF36-4 C2893 |
| | Coprococcus comes C2152 |
| | Dorea longicatena C2131 |
| | Erysipelotrichaceae bacterium GAM147 C2844 |
| | Firmicutes bacterium AF12-30 C2644 |
| | Ruminococcus sp. OF03-6AA C2904 |
| 255 | Blautia obeum C2129 |
| | Clostridium sp. AF36-4 C2893 |
| | Coprococcus comes C2152 |
| | Erysipelotrichaceae bacterium GAM147 C2844 |
| | Firmicutes bacterium AF12-30 C2644 |
| | Ruminococcus sp. OF03-6AA C2904 |
| 256 | Blautia obeum C2129 |
| | Clostridium sp. AF36-4 C2893 |
| | Coprococcus comes C2152 |
| | Dorea longicatena C2131 |
| | Erysipelotrichaceae bacterium GAM147 C2844 |
| | Firmicutes bacterium AF12-30 C2644 |
| 257 | Blautia obeum C2129 |
| | Clostridium sp. AF36-4 C2893 |
| | Coprococcus comes C2152 |
| | Dorea longicatena C2131 |
| | Erysipelotrichaceae bacterium GAM147 C2844 |
| | Ruminococcus sp. OF03-6AA C2904 |
| 258 | Blautia obeum C2129 |
| | Dorea longicatena C2131 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 259 | Coprococcus comes C2152 |
| | Dorea longicatena C2131 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 260 | Blautia obeum C2129 |
| | Coprococcus comes C2152 |
| | Erysipelotrichaceae bacterium GAM147 C2844 |
| | Firmicutes bacterium AF12-30 C2644 |
| | Ruminococcus lactaris C2149 |
| | Ruminococcus sp. OF03-6AA C2904 |
| 261 | Blautia obeum C2129 |
| | Coprococcus comes C2152 |
| | Dorea longicatena C2131 |
| | Erysipelotrichaceae bacterium GAM147 C2844 |
| | Firmicutes bacterium AF12-30 C2644 |
| | Ruminococcus lactaris C2149 |
| 262 | Blautia obeum C2129 |
| | Coprococcus comes C2152 |
| | Dorea longicatena C2131 |
| | Erysipelotrichaceae bacterium GAM147 C2844 |
| | Ruminococcus lactaris C2149 |
| | Ruminococcus sp. OF03-6AA C2904 |
| 263 | Blautia obeum C2129 |
| | Coprococcus comes C2152 |
| | Dorea longicatena C2131 |
| | Erysipelotrichaceae bacterium GAM147 C2844 |
| | Firmicutes bacterium AF12-30 C2644 |
| | Ruminococcus sp. OF03-6AA C2904 |
| 264 | Erysipelotrichaceae bacterium GAM147 C2844 |
| | Dorea sp. AM58-8 C2913 |
| | Bifidobacterium bifidum C0005 |
| | Clostridium sp. AF36-4 C2893 |
| | Ruminococcus lactaris C2149 |
| | Firmicutes bacterium AF12-30 C2644 |
| | Ruminococcus sp. OF03-6AA C2904 |
| | Dorea longicatena C2131 |
| | Blautia obeum C2129 |
| | Coprococcus comes C2152 |
| | Bifidobacterium catenulatum C0014 |
| | Blautia sp. AF19-10LB C2906 |
| 265 | Ruminococcus sp. OF03-6AA C2904 |
| | Dorea longicatena C2131 |
| | Blautia obeum C2129 |
| | Coprococcus comes C2152 |
| | Bifidobacterium catenulatum C0014 |
| | Blautia sp. AF19-10LB C2906 |
| 266 | Ruminococcus sp. OF03-6AA C2904 |
| | Dorea longicatena C2131 |
| | Blautia obeum C2129 |
| | Coprococcus comes C2152 |
| | Bifidobacterium catenulatum C0014 |
| | Blautia sp. AF19-10LB C2906 |
| | Erysipelotrichaceae bacterium GAM147 C2844 |
| 267 | Dorea sp. OM07-5 C2890 |
| | Faecalibacterium prausnitzii C2184 |
| | Dorea longicatena C2413 |
| | Anaerobutyricum hallii C2206 |
| | Faecalibacterium prausnitzii C2650 |
| | Faecalibacterium prausnitzii C2651 |
| | Anaerostipes hadrus C2144 |
| | Dorea formicigenerans C2197 |
| | [Ruminococcus] torques C2636 |
| | Coprococcus catus C2881 |
| | Faecalibacterium sp. AF28-13AC C2810 |
| | [Clostridium] amygdalinum C2887 |
| | Roseburia inulinivorans C2207 |
| | Asaccharobacter celatus Cl952 |
| 268 | Erysipelotrichaceae bacterium GAM147 C2844 |
| | Dorea sp. AM58-8 C2913 |
| | Bifidobacterium bifidum C0005 |
| | Clostridium sp. AF36-4 C2893 |
| | Dorea longicatena C2131 |
| | Firmicutes bacterium AF12-30 C2644 |
| | Ruminococcus sp. OF03-6AA C2904 |
| | Coprococcus comes C2152 |
| | Bifidobacterium catenulatum COO 14 |
| | Blautia sp. AF19-10LB C2906 |
| | Dorea formicigenerans C2197 |
| | [Ruminococcus] torques C2636 |
| | Coprococcus catus C2881 |
| 269 | Erysipelotrichaceae bacterium GAM147 C2844 |
| | Dorea sp. AM58-8 C2913 |
| | Dorea longicatena C2131 |
| | Bifidobacterium catenulatum COO 14 |
| | Dorea formicigenerans C2197 |
| | Coprococcus comes C2152 |
| | Coprococcus catus C2881 |
| 270 | Erysipelotrichaceae bacterium GAM147 C2844 |
| | Dorea sp. AM58-8 C2913 |
| | Firmicutes bacterium AF12-30 C2644 |
| | Ruminococcus sp. OF03-6AA C2904 |
| | Dorea longicatena C2131 |
| | Blautia obeum C2129 |
| | Dorea sp. OM07-5 C2890 |
| | Coprococcus comes C2152 |
| | Dorea longicatena C2413 |
| | Faecalibacterium prausnitzii C2650 |
| | Blautia sp. AF19-10LB C2906 |
| | [Ruminococcus] torques C2636 |
| 271 | Erysipelotrichaceae bacterium GAM147 C2844 |
| | Dorea sp. AM58-8 C2913 |
| | Bifidobacterium bifidum C0005 |
| | Clostridium sp. AF36-4 C2893 |
| | Ruminococcus lactaris C2149 |
| 272 | Erysipelotrichaceae bacterium GAM147 C2844 |
| | Dorea sp. AM58-8 C2913 |
| | Ruminococcus lactaris C2149 |
| | Dorea formicigenerans C2197 |
| | [Clostridium] amygdalinum C2887 |
| | Roseburia inulinivorans C2207 |
| | Asaccharobacter celatus C1952 |
| 273 | Erysipelotrichaceae bacterium GAM147 C2844 |
| | Dorea sp. AM58-8 C2913 |
| | Bifidobacterium bifidum C0005 |
| | Clostridium sp. AF36-4 C2893 |
| | Ruminococcus lactaris C2149 |
| | Dorea formicigenerans C2197 |
| | [Clostridium] amygdalinum C2887 |
| | Roseburia inulinivorans C2207 |
| | Asaccharobacter celatus C1952 |
| 274 | Erysipelotrichaceae bacterium GAM147 C2844 |
| | Dorea sp. AM58-8 C2913 |
| | Ruminococcus lactaris C2149 |
| 275 | Bifidobacterium bifidum C0005 |
| | Bifidobacterium catenulatum C0014 |
| | Bifidobacterium pseudocatenulatum C0013 |
| 276 | Blautia luti C2436 |
| | Blautia obeum C2129 |
| | Blautia obeum C2901 |
| | Blautia sp. AF19-10LB C2906 |
| 277 | Blautia luti C2436 |
| | Blautia obeum C2129 |
| | Blautia obeum C2901 |
| | Blautia sp. AF19-10LB C2906 |
| | Blautia sp. KGMB01111 C3003 |
| | Blautia sp. TF11-31AT C2841 |
| | Blautia wexlerae C2171 |
| 278 | Clostridium sp. AF20-17LB C2921 |
| | Clostridium sp. AF23-8 C2908 |
| | Clostridium sp. AF34-13 C2653 |
| | Clostridium sp. AF36-4 C2893 |
| | Clostridium sp. AM18-55 C2845 |
| | Clostridium sp. AM49-4BH C2934 |
| | Clostridium sp. OF10-22XD C2132 |
| 279 | Collinsella aerofaciens C1933 |
| | Collinsella bouchesdurhonensis C1956 |
| | Collinsella sp. TM05-38 C1984 |
| 280 | Coprococcus catus C2881 |
| | Coprococcus comes C2152 |
| | Coprococcus eutactus C2642 |
| 281 | Dorea formicigenerans C2197 |
| | Dorea longicatena C2131 |
| | Dorea longicatena C2413 |
| | Dorea sp. AM58-8 C2913 |
| | Dorea sp. OM07-5 C2890 |
| 282 | Eubacterium ramulus C2442 |
| | Eubacterium ramulus C2852 |
| | Eubacterium saphenum ATCC 49989 C2183 |
| | Eubacterium ventriosum C2128 |
| 283 | Faecalibacterium prausnitzii C2138 |
| | Faecalibacterium prausnitzii C2184 |
| | Faecalibacterium prausnitzii C2650 |
| | Faecalibacterium prausnitzii C2651 |
| | Faecalibacterium prausnitzii C2863 |
| | Faecalibacterium prausnitzii C2864 |
| | Faecalibacterium sp. AF28-13AC C2810 |
| 284 | Firmicutes bacterium AF12-30 C2644 |
| | Firmicutes bacterium AF22-6AC C2933 |
| | Firmicutes bacterium AF25-13AC C2695 |
| | Firmicutes bacterium AM41-11 C2946 |
| | Firmicutes bacterium TM09-10 C2909 |
| 284 | Roseburia inulinivorans C2207 |
| | Roseburia sp. AM59-24XD C2936 |
| | Roseburia sp. OM04-15AA C2892 |
| 285 | Ruminococcus callidus C2440 |
| | Ruminococcus lactaris C2149 |
| | Ruminococcus sp. AF31-8BH C2903 |
| | Ruminococcus sp. AM42-11 C2945 |
| | Ruminococcus sp. KGMB03662 C2557 |
| | Ruminococcus sp. OF03-6AA C2904 |
| 286 | Flavonifractor plautii C2284 |
| | [Clostridium] scindens C2143 |
| | [Clostridium] bolteae C2137 |
| 287 | Flavonifractor plautii C2284 |
| | [Clostridium] scindens C2143 |
| | [Clostridium] bolteae C2137 |
| | Blautia hansenii C3044 |
| | [Clostridium] clostridioforme C2275 |
| 288 | Erysipelotrichaceae bacterium GAM147 C2844 |
| | Ruminococcus sp. OF03-6AA C2904 |
| | Blautia sp. AF19-10LB C2906 |
| 289 | Erysipelotrichaceae bacterium GAM147 C2844 |
| | Ruminococcus sp. OF03-6AA C2904 |
| | Blautia sp. AF19-10LB C2906 |
| | Firmicutes bacterium AF12-30 C2644 |
| 290 | Dorea longicatena C2131 |
| | Coprococcus comes C2152 |
| | Blautia obeum C2129 |
| | Faecalibacterium prausnitzii C2184 |
| | Dorea longicatena C2413 |
| 291 | Dorea longicatena C2131 |
| | Coprococcus comes C2152 |
| | Blautia obeum C2129 |
| | Faecalibacterium prausnitzii C2184 |
| | Dorea longicatena C2413 |
| | [Ruminococcus] torques C2636 |
| 292 | Erysipelotrichaceae bacterium GAM147 C2844 |
| | Dorea longicatena C2131 |
| | Coprococcus comes C2152 |
| | Blautia obeum C2129 |
| | Faecalibacterium prausnitzii C2184 |
| | Dorea longicatena C2413 |
| 293 | Erysipelotrichaceae bacterium GAM147 C2844 |
| | Ruminococcus sp. OF03-6AA C2904 |
| | Blautia sp. AF19-10LB C2906 |
| | Firmicutes bacterium AF12-30 C2644 |
| | Dorea longicatena C2131 |
| | Coprococcus comes C2152 |
| | Blautia obeum C2129 |
| | Faecalibacterium prausnitzii C2184 |
| | Dorea longicatena C2413 |
| | [Ruminococcus] torques C2636 |
| 294 | Erysipelotrichaceae bacterium GAM147 C2844 |
| | Dorea longicatena C2131 |
| | Coprococcus comes C2152 |
| | Blautia obeum C2129 |
| | Faecalibacterium prausnitzii C2184 |
| | Dorea longicatena C2413 |
| | [Ruminococcus] torques C2636 |

### Example 11: Gene Expression Analysis of Microbial Treatment in Co-Culture

Live biotherapeutic compositions as provided herein, including the exemplary combinations of microbes 1 to 294, as described in Table 9, Example 10 and Table 42, Example 25, are evaluated in co-culture for immunomodulatory effects. Live biotherapeutics are co-cultured with human colonic cells (CaCo2) to investigate the effects of the bacteria on the host. Live biotherapeutic compositions are also co-cultured on CaCo2 cells that were stimulated with Interleukin 1 (IL1) to mimic the effect of the bacteria in an inflammatory environment. The effects in both scenarios are evaluated through gene expression analysis either by PCR or by next generation sequencing approaches.

### Cytokine Production in THP-1 Cells Induced by Live Biotherapeutics

Live biotherapeutic compositions as provided herein, including for example the exemplary combinations of microbes 1 to 294, Table 9, Example 10 and Table 42, Example 25, and single bacterial strains are evaluated alone and in combination with lipopolysaccharide (LPS) on cytokine production in THP-1 cells, a model cell line for monocytes and macrophages.

THF-1 cells are differentiated into M0 medium for 48 h with 5 ng/mL phorbol-12-myristate-13-acetate (PMA). These cells are subsequently incubated with the live biotherapeutic composition at a final concentration of 10⁸/ml, with or without the addition of LPS at a final concentration of 100 ng/ml. Alternatively, the bacterial cells are centrifuged, and the resulting supernatant is added to the THF-1 cell preparation. The bacteria are then washed off and the cells allowed to incubate under normal growing conditions for 24 h. The cells are then spun down and the resulting supernatant is analyzed for cytokine content using a Luminex 200 analyzer or equivalent method.

### Cytokine Production in Immature Dendritic Cells Induced by Live Biotherapeutic Compositions

Live biotherapeutic compositions as provided herein, including the exemplary combinations of microbes 1 to 294, as described in Table 9, Example 10, and Table 42, Example 25 and single bacterial strains are evaluated alone and in combination with LPS on cytokine production in immature dendritic cells. A monocyte population is isolated from peripheral blood mononuclear cells (PBMCs). The monocyte cells are subsequently differentiated into immature dendritic cells. The immature dendritic cells are plated out at 200,000 cells/well and incubated with the live biotherapeutic composition at a final concentration of 10⁷/ml in RPMI media, with the optional addition of LPS at a final concentration of 100 ng/ml. Alternatively, the bacterial cells are centrifuged, and the resulting supernatant is added to the dendritic cell preparation. The negative control involves incubating the cells with RPMI media alone and positive controls incubating the cells with LPS at a final concentration of 100 ng/ml. The cytokine content of the cells is then analyzed.

### Cytokine Production and Analysis in PBMCs

Peripheral blood mononuclear cells (PBMC's) are isolated from subject blood using a standard kit and stored in liquid nitrogen at 1×10⁶ cells per mL until use. Prior to storage, PBMC's may be processed using flow sorting or an antibody spin separation kit to select for a certain purified lymphocyte subpopulation, such as T cells.

PBMCs are thawed at 37° C. and then transferred to a growth medium consisting of RPMI-1640 (Lonza, Switzerland), with 10% heat inactivated FCS added, as well as 0.1% penicillin-streptavidin, 1% L-glutamine, and DNase at 10 mg/mL to inhibit aggregation. Cells are centrifuged at 200×g for 15 minutes and then counted using trypan blue and spread into 24 well plates at 1×10⁶ cells per well (1 mL per well) (Kechaou et al. (2013) Applied and Environmental Microbiology 79:1491-1499; Martin et al. (2017) Frontiers in Microbiology 8:1226).

An overnight bacterial culture is inoculated using a pre-stocked isolated bacterial strain. This strain is grown at 37° C. for 10 to 20 hours in a YBHI medium with added cellobiose (1 mg/mL), maltose (1 mg/mL) and cysteine (0.5 mg/mL) in an anaerobic chamber filled with 85% nitrogen, 10% carbon dioxide, and 5% hydrogen (Martin et al., 2017). The growth medium may also be Reinforced Clostridial Medium (RCM) (Thermo Fisher, USA), which may also be supplemented with cysteine (0.5 mg/mL) or arginine (1 mg/mL).

At the end of the anaerobic culture, the culture supernatant and bacterial cells alone are saved for co-culture with PBMC's. Microbial culture supernatant is saved directly after centrifugation at −80° C. Cells are saved by washing with phosphate buffered saline (PBS) and then storing in PBS with 15% glycerol. Bacteria are quantified using phase contrast microscopy and stored at a final concentration of 10⁵ or 10⁶ cells per mL (Haller et al. (2000) Infection and Immunity 68; Rossi et al. (2015) Scientific Reports 6:18507) at −80° C. Bacteria may also be pasteurized prior to storage by treatment at 70° C. for 30 minutes (Plovier et al. (2017) Nature Medicine 23:107-113).

Prior to co-culture, supernatant is thawed on ice and 200 μL of supernatant is diluted in 1 mL of total volume of PBMC growth medium. Microbial growth medium is used as a negative control. This 1 mL is added to the 1 mL of PBMC in each well, resulting in a 10% final level of microbial culture supernatant in a 2 mL culture containing 1×10⁶ PBMCs. Each combination of PBMCs and supernatant is performed in duplicate or triplicate.

Prior to co-culture, bacteria are thawed on ice and then washed at 4° C. with PBMC growth medium. 1 mL of the bacterial suspension is added to the 1 mL of PBMC culture in each well of the plate, resulting in a final 2 mL culture containing 1×10⁶ PBMC's and 1×10⁵ or 1×10⁶ (potentially pasteurized) bacteria.

The co-culture of PBMC's and supernatant or purified bacteria is incubated for 2, 6, 16, 24, or 48 hours at 37° C. in 10% carbon dioxide.

After co-culture, the supernatant is harvested and treated with a protease inhibitor (Complete EDTA-Free protease inhibitor, Roche Applied Bioscience) to protect cytokines and stored directly at −80° C. for cytokine profiling. The pelleted cells are treated with RNAlater (Thermo Fisher, USA) and saved for RNA sequencing.

Cytokine analysis is performed on saved co-culture supernatant using ELISA or a Luminex system. Cytokines measured may include but are not limited to, IL-10, IL-2, and IFN-gamma.

RNA sequencing is performed on PBMC's saved in RNAlater post co-culture. Standard pseudo-alignment is performed using Kallisto (Bray et al. (2016) Nature Biotechnology 34:525-527) and differential expression is analyzed using DESeq2 (Love et al. (2014) Genome Biology 15:550) to identify differential expression between different microbes and different PBMC donors.

Statistical analyses are performed to identify microbes that exhibit desired immunomodulatory effects in vitro, which include but are not limited to inducing production of IFN-gamma and lowering expression of genes associated with T cell exhaustion (PD1, CTLA4, VISTA, TIM3, TIGIT, LAG3).

### Example 12: Genetic Modification of Therapeutic Microbes

Microbes of interest, including microbes as provided herein, for example, as listed in Table 1, 4, 7 or 8, including bacteria from all the genuses listed herein, and including the combinations of microbes as provided herein, for example, the exemplary combinations 1 to 294 as described in Table 9, Example 10, and Table 42, Example 25, or as identified from the in vivo and ex vivo analyses described in Example 10 and Example 11, are interrogated or investigated to identify mechanisms of action, and the discovered mechanisms are leveraged using a genetic modification or modifications to amplify the microbe's therapeutic effect.

In alternative embodiments, this is accomplished in two stages. First, complementary bioinformatic and experimental approaches are used to identify the genes within a microbe of interest responsible for its therapeutic effect. Second, synthetic biology techniques are used to engineer over-expression of the identified genes within the original organism of discovery or inserted for overexpression in the genome of a chassis organism. Chassis organisms include any microbe as described herein, including genuses of bacteria as provided herein, and also include bacteria as listed in Tables 1, 3, 4, 5, 6, 7 and/or 8, including Bacillus subtilis, Escherichia coli Nissle, or any microbes listed in the combinations as provided herein in Table 9 and Table 42, Example 25, or the original organism of interest itself.

In alternative embodiments, microbes as provided herein are genetically modified to increase expression of existing therapeutically effective genes, or to install extra copies of these genes, or to install into a microbe lacking these functions any one of these genes. Methods for genetic engineering/augmenting a microbe of interest, for example, a gut microbe, to alter expression of existing therapeutically effective genes or to install extra copies of said genes or to install said genes in a microbe lacking these functions are numerous in the art. Techniques applied to gut microbes and related organisms for experimental gene disruption, gene replacement or gene expression modulation include CRISPR-Cas9 genome editing (Bruder et al (2016) Applied and Environmental Microbiology 82:6109-6119), bacterial conjugation (Cuiv et al (2015) Nature Scientific Reports 5:13282; Ronda et al. (2019) Nature Methods 16:167-170), gene replacement mutagenesis by homologous recombination (Cartman et al (2012) Applied Environmental Microbiology 78:4683-4690; Heap et al (2007) Journal of Microbiological Methods 70:452-464), random transposon mutagenesis (Cartman and Minton (2010) Applied Environmental Microbiology 76:1103-1109), and antisense-based gene expression attenuation (Forsyth et al (2002) Molecular Microbiology 43:1387-1400; Kedar et al (2007) Antimicrobial Agents and Chemotherapy 51:1708-1718.

Genes of interest inserted into microbes as provided herein, or whose expression is increased in microbes as provided herein, can be engineered to immediately follow and be under inducible control by various promotor elements that are functional in gut microbes. Highly inducible and controllable promoter elements are available for bacteria in the gram-negative genus Bacteroides (Lim et al (2017) Cell 169:547-558; Bencivenga-Barry et al (2019) Journal of Bacteriology doi: 10.1128/JB.00544-19). Some of these are responsive to various diet-derived polysaccharides, while those often most useful for use for inducible function determination in animal models such as mouse rely on induction by tetracycline derivatives like anhydrotetracycline at sub-bactericidal levels. Anhydrotetracycline can be employed as an inducer for engineered promoters in gut Clostridia (Dembek et al (2017) Frontiers of Microbiology 8:1793). Promoters that respond to bile acids are identified in gram-positive gut Clostridium species (Wells and Hyemon (2000) Applied Environmental Microbiology 66:1107-1113) and in Eubacterium species (Mallonee et al. (1990) Journal of Bacteriology 172:7011-7019. Also, inducible promoters that respond to sugars such as lactose (Banerjee et al (2014) Applied Environmental Microbiology 80-2410-2416) and arabinose (Zhang et al (2015) Biotechnology for Biofuels 8:36) are identified and useful in related Clostridial species. Genes inserted in exemplary recombinant bacterium can be induced under low-oxygen conditions from promoters driven by transcriptions factors such as FNR (fumarate and nitrate reductase) (Oxer et al (1991) Nucleic Acids Research, 19, 11: 2889-2892). Genes of interest inserted in microbes as provided herein can also be engineered to immediately follow and be under constitutive control by various promotor elements that are functional in gut microbes. Constitutive promoter libraries and promoter-RBS (ribosome binding site) pairs have been created for bacteria in the gram-negative genus Bacteroides (Mimee et al (2015) Cell Syst. 1, 62-71) and computational models have been developed from Bacillus subtilis promoter sequences data sets for promoter prediction in Gram-positive bacteria (Coelho et al (2018) Data Br. 19, 264-270).

### Engineering of Metabolic Pathways in Therapeutic Microbes

In one embodiment, an organism used to practice embodiments as provided herein is genetically modified to overexpress a pathway for production of any short chain fatty acid (SCFA), including butyrate or butyric acid, propionate and acetate. Butyric acid is naturally produced in many gut microorganisms and is derived from two molecules of acetyl-CoA, a central metabolic intermediate that is ubiquitous in microorganisms. In one embodiment, the native pathway is overexpressed, for example, as discussed herein. In another embodiment, a heterologous pathway is constructed by introducing one or more genes from a different organism, including all genes derived from different organisms. Condensation of two acetyl-CoA molecules is catalyzed by a ketothiolase (EC:2.3.1.9), such as the atoB gene from Escherichia coli, to produce one molecule of acetoacetyl-CoA (Sato et al. (2007) J. Biosci. Bioengineer. 103:38-44). Alternative candidates are obtained by Basic Local Alignment Search Tool (BLAST) search of this sequence (Altschul et al. (1997) Nuc. Acids. Res. 25:3389-3402), obtaining homologous genes either known or predicted to encode similar enzyme function. Exemplary gene candidates are obtained using the following GenBank accession numbers.

| | | |
|---|---|---|
| atoB | Escherichia coli | NP_416728.1 |
| yqeF | Escherichia coli | NP_417321.2 |
| phaA | Cupriavidus necator | YP_725941 |
| bktB | Cupriavidus necator | AAC3 8322.1 |
| thiA | Clostridium acetobutylicum | NP_349476.1 |
| thiB | Clostridium acetobutylicum | NP_149242.1 |

The second step in the pathway involves reduction of acetoacetyl-CoA to 3-hydroxybutyryl-CoA by a hydroxyacyl-CoA dehydrogenase (EC:1.1.1.35), such as that encoded by hbd in Clostridium acetobutylicum (Atsumi et al. (2008) Metab. Eng. 10(6):305-311). Similarly, to above, alternate candidates are identified in the literature or by BLAST. Exemplary candidates are as follows.

| | | |
|---|---|---|
| paaH | Escherichia coli | NP_415913.1 |
| hbd | Clostridium acetobutylicum | NP_349314.1 |
| hbd | Pseudomonas putida | KT2440 NC_002947.4 |
| RSP_3970 | Rhodobacter sphaeroides 2.4.1 | YP_345236.1 |

The next step is the dehydration of 3-hydroxybutyryl-CoA to crotonyl-CoA by an enoyl-CoA hydratase, also known as crotonase (EC:42.1.55), such as that encoded by the crt gene of Clostridium acetobutylicum (Kim et al. (2014) Biochem. Biophys.

Res. Commun. 451:431-435) or the homologs listed below.

| | | |
|---|---|---|
| crt | Clostridium acetobutylicum | NC_003030.1 |
| echA18 | Mycobacterium bovis AF2122/97 | NC_002945.4 |
| maoC | Escherichia coli | NP_415905.1 |
| crt | Bacillus thuringiensis | NC_005957.1 |

Next, crotonyl-CoA is reduced to butyryl-CoA through the action of an enoyl-CoA reductase (EC:1.3.1.38 or EC:1.3.1.44), such as that encoded by the bcd gene of Clostridium acetobutylicum (Boynton et al. (1996) J. Bacteriol. 178:3015-3024). Activity of this enzyme can be enhanced by expressing bcd in conjunction with expression of the C. acetobutylicum etfAB genes, which encode an electron transfer flavoprotein. Several eukaryotic enzymes with this activity have also been identified, such as TER from Euglena gracilis, that upon removal of the mitochondrial targeting leader sequence have demonstrated superior activity in E. coli (Hoffmeister et al. (2005) J. Biol. Chem. 280:4329-4338). Protein sequences for these and other exemplary sequences can be obtained using the following GenBank accession numbers.

| | | |
|---|---|---|
| bed | Clostridium acetobutylicum | NP_34.9317.1 |
| etfA | Clostridium acetobutylicum | NP_349315.1 |
| etfB | Clostridium acetobutylicum | NP_349316.1 |
| TER | Euglena gracilis | Q5EU90.1 |
| TDE0597 | Treponema denticola | NP 97.1211.1 |

The final step of this pathway is CoA removal from butyryl-CoA to generate butyric acid. Although numerous CoA hydrolases occur in most bacteria, for example, tesB from E. coli ((Naggert et al. (1991) J. Biol. Chem. 266:11044-11050), it is desirable to recover energy from hydrolysis of the thioester bond in the form of ATP. The sucCD complex of E. coli (EC:6.2.1.5) is one example of this, known to catalyze the conversion of succinyl-CoA and ADP to succinate and ATP (Buck et al. (1985) Biochem. 24:6245-6252). Another example is sucD, succinic semialdehyde dehydrogenase, from Porphyromonas gingivalis (Yim et al. (2011) Nat. Chem. Biol. 7:445-452). Another option, using phosphotransbutylase/butyrate kinase (EC:2.3.1.19, EC:2.7.2.7), is catalyzed by the gene products of buk1, buk2, and ptb from C. acetobutylicum (Walter et al. (1993) Gene 134:107-111) or homologs thereof. Finally, an acetyltransferase capable of transferring the CoA group from butyryl-CoA to acetate can be applied (EC:2.8.3.9), such as Cat3 from C. kluyveri (Sohling and Gottschalk (1996) J. Bacteriol. 178:871-880). Protein sequences for these and other exemplary sequences can be obtained using the following GenBank accession numbers.

| | | |
|---|---|---|
| ptb | Clostridium acetobutylicum | NP 349676 |
| buk1 | Clostridium acetobutylicum | NP 349675 |
| buk2 | Clostridium acetobutylicum | Q97II1 |
| sucC | Escherichia coli | NP_415256.1 |
| sueD | Escherichia coli | AAC73 823.1 |
| cat3 | Clostridium kluyveri | EDK35586.1 |
| tesB | Escherichia coli | NP_414986 |

In another embodiment, a microbe used to practice embodiments as provided herein is genetically modified to metabolize bile acids, also referred to as bile salts to indicate the predominant form at neutral pH, that are produced in the liver and present in the gut at about 1 mM concentration. Two such types of bile acid conversion processes are catalyzed by bacteria. The first is deconjugation, which removes either taurine or glycine that is frequently found conjugated to bile acids (Ridlon et al. (2016) Gut Microbes 7:22-39; Masuda et al. (1981) Microbiol. Immunol. 25:1-11). This is catalyzed by bile salt hydrolase (BSH) enzymes (EC:3.5.1.24), which are widespread in many gut bacteria. Some BSHs have broad substrate specificity, while others are very specific for a particular bile salt. The substrate range of a BSH of interest is determined by assay of purified BSH or crude lysates from the native host, on a panel of glycine and taurine conjugated bile salts (Jones et al. (2008) Proc. Nat. Acad. Sci. USA 105:13580-13585). To enhance the activity and substrate range of bile salt deconjugation in the engineered microbe, native BSHs of interest and/or heterologous genes from other microbes are introduced. Exemplary genes are listed below. Still others are found by GenBank search or BLAST of these sequences to identify homologs.

| | | |
|---|---|---|
| bsh | Bifidobacterium longum | AF148138.1 |
| bsh | Bifidobacterium animalis | AY530821.1 |
| bsh | Enterococcus faecalis | GG688660.1 |
| bsh3 | Lactobacillus plantarum | ACL98170.1 |
| cbh2 | Bacteroides vulgatis | RIB33278.1 |
| cbah | Clostridium butyricum | EEP54620.1 |

The other type of bile acid metabolism introduced into a microbe used to practice embodiments as provided herein is capable of converting primary to secondary bile acids, which entails removal of the 7-alpha-hydroxy or 7-beta hydroxy group from the primary bile acid; for example, the conversion of cholic acid to deoxycholic acid or chenodeoxycholic acid to lithocholic acid. The archetype pathway for this process is encoded by the bai gene cluster in Clostridium scindens (Coleman et al. (1987) J. Bacteriol. 169:1516-1521; Ridlon et al. (2006) J. Lipid. Res. 47:241-259) and has been well characterized. In addition, a functional C. scindens dihydroxylation was established in Clostridium sporogenes (Funabashi et al. (2019) BioRxiv). The first step is a bile acid-CoA ligase (baiB, EC:6.2.1.7) to activate the molecule for the subsequent reaction steps. Next, an alcohol dehydrogenase (baiA, EC:1.1.1.395) oxidizes the 3-hydroxyl to a keto group. An NADH:flavin oxidoreductase then introduces a double bond into the ring by either baiCD (EC:1.3.1.115) or baiH (EC:1.3.1.116), depending on the substrate. The coA is then removed or transferred to another primary bile acid by a CoA transferase (baiF, EC:2.8.3.25). The 7-alpha or 7-beta-hydroxy group is then removed by a dehydratase (baiE or baiI, respectively, EC:4.2.1.106) to form a second double bond in a conjugated position to the other one. Enzymes encoded by baiH and baiCD then serve to reduce the double bonds consecutively, and finally the alcohol dehydrogenase reduces the 3-keto back to a hydroxyl. High bile acid dihydroxylation activity has also been observed in Eubacterium sp. strain VPI 12708, Eubacterium sp. strain Y-1113, Eubacterium sp. strain I-10, Eubacterium sp. strain M-18, Eubacterium sp. strain TH-82, Clostridium sp. strain TO-931, and Clostridium sp. strain HD-17. Homologs for some of the bai genes have been identified in these organisms (Doemer et al. (1997) Appl. Environ. Microbiol. 63:1185-1188), and thus represent alternate gene candidates. Homologs of all essential genes for pathway function were also identified in Clostridium hylemonae DSM 15053, Dorea sp. D7, and a novel Firmicutes bacterium (Das et al. (2019) BMC Genomics 20:517).

To introduce the conversion pathway into the genetically modified host, the following C. scindens genes or suitable homologs are expressed: baiA, baiB, baiCD, baiE, baiF, and baiH. In some embodiments, the baiG gene, encoding a transporter, is also expressed. In other embodiments, the baiI gene predicted to encode a delta-5-ketoisomerase, is introduced in order to enable dihydroxylation of secondary bile acids requiring this step.

Tryptophan derivatives are produced by many microbes, including gut bacteria, and have been implicated in strengthening the epithelial cell barrier and modulating the expression of pro-inflammatory genes by T cells in the GI tract (Bercik et al. (2011) Gastroenterology 141:599-609). A gut microbe is engineered to overexpress one or more tryptophan derivatives by either overexpressing native genes or introducing heterologous genes described below.

In one embodiment, a microbe used to practice embodiments as provided herein is engineered to convert tryptophan to indole by introduction of a tryptophanase, such as that encoded by the tnaA gene of E. coli (Li and Young (2013) Microbiology 159:402-410). Other candidates are found by literature search or BLAST of the sequence to find homologs, as exemplified by the following:

| | | |
|---|---|---|
| tnaA | Escherichia coli | NP_415256.1 |
| tnaA | Bacteroides thetaiotamicron | NP_810405.1 |
| tnaA | Vibrio tasmaniensis | LGP32VS_RS05915 |
| tnaA | Treponema denticola | TDE0251 |

In another embodiment, a microbe used to practice embodiments as provided herein is engineered to convert tryptophan to indoleacetate. This pathway begins with a tryptophan aminotransferase (EC:2.6.1.27) such as that encoded by the Taml gene of Ustilago maydis (Zuther et al. (2008) Mol. Microbiol. 68:152-172), which uses a-ketoglutarate as the amino acceptor and produces indolepyruvate. Although a microbial sequence for this enzyme is not currently in GenBank, activity has been reported in Clostridium sporogenes (O'Neil et al. (1968) Arch. Biochem. Biophys. 127:361-369). Alternatively, a deaminating tryptophan oxidase (EC:1.3.3.10) such as that encoded by the vioA gene of Chromobacterium violaceum (August et al. (2000) J. Mol. Microbiol. Biotechnol. 2:513-519) uses molecular oxygen to oxidize and deaminate tryptophan to produce indolepyruvate. Alternative candidates include those indicated as follows:

| | | |
|---|---|---|
| vioA | Chromobacterium violaceum | CV_RS16140 |
| WP_133678757 | Paludibacterium purpuratum | WP_133678757.1 |
| WP_034786442 | Janthinobacterium lividum | WP_034786442.1 |

The next gene to be introduced encodes an indolepyruvate decarboxylase (EC:4.1.1.74), which produces indole-3-acetaldehyde from indolepyruvate. An example is the ipdC gene from Enterobacter cloacae (Koga et al. (1991) Mol. Gen. Genet. 226:10-16). Other exemplary genes can be accessed by the GenBank accession numbers listed below:

| | | |
|---|---|---|
| ipdC | Enterobacter cloacae | WP_013098183.1 |
| CFNIH1_ | Citrobacter freundii | CFNIH1_RS23020 |
| RS23020 | | |
| ipdC | Rhodopseudomonas palustris | TX73_RS15890 |
| | CGA009 | |
| ipdC | Azospirillum brasilense | AMK58_RS11560 |

Indole-3-acetaldehyde is then oxidized to indoleacetate by an aldehyde dehydrogenase (EC:1.2.1.3), such as that encoded by the aldA gene of Pseudomonas syringae (McClerklin et al. (2018) PLoS Pathog. 14:e1006811). Numerous aldehyde dehydrogenases exist, though the best candidates are those homologous to this aldA or others with known activity on indole-3-aldehyde or similar molecules. Exemplary gene candidates can be accessed by the GenBank accession numbers listed below:

| | | |
|---|---|---|
| ald A | Pseudomonas syringae | PSPTO_0092 |
| CFNIH1_RS23020 | Citrobacter freundii | CFNIH1_RS23020 |
| WP_005887684.1 | Pseudomonas coronafaciens | WP_005887684.1 |
| SPOG_02634 | Schizosaccharomyces cryophilus | OY26 SPOG_02634 |

In another embodiment, a tryptophan decarboxylase (EC:4.1.1.28) is introduced into a microbe used to practice embodiments as provided herein to produce tryptamine. This activity is rare among bacteria, but two such enzymes have recently been identified: CLOSPO_02083 from Clostridium sporogenes and RUMGNA_01526 from Ruminococcus gnavus (Williams et al. (2014) Cell Host Microbe 16:495-503).

In another embodiment, the pathway to produce indolepropionate (IPA) is introduced into the genetically modified microbe. IPA has been implicated in intestinal barrier fortification by engaging the pregnane X receptor (Venkatesh et al. (2014) Immunity 41:296-310) and is known to be synthesized by a small number of gut bacteria (Elsden et al. (1976) Arch. Microbiol. 107:283-288). However, the pathway for its synthesis is uncharacterized. The genes encoding this pathway have recently been discovered in Clostridium sporogenes, enabling a pathway to be proposed. Indolepyruvate, synthesized as described above, is reduced to indolelactate which is then dehydrated to produce indoleacrylate. Finally, indoleacrylate is reduced to IPA by an acyl-CoA dehydrogenase. These are encoded by the fldH, fldBC, and acdA genes in C. sporogenes, respectively (Dodd et al. (2017) Nature 551:648-652). Homologs of these genes in other microbes are also candidates for expression, found by BLAST of the C. sporogenes genes.

In another embodiment, a microbe used to practice embodiments as provided herein is engineered to consume a sugar or polysaccharide, for example, a cellobiose, which is a reducing sugar consisting of two β-glucose molecules linked by a β(1→4) bond that is recalcitrant to catabolism by most gut microbes. Consumption of cellobiose first requires a specific enzyme II complex (EC:2.7.1.205) of the phosphotransferase system (PTS), such as the celABC operon in E. coli (Keyhani et al. (2000) J. Biol. Chem. 275:33091-33101). When expressed in a heterologous host, this component functions together with the native PTS machinery to import and phosphorylate cellobiose to generate cellobiose-6-phosphate. Alternate candidates for this step are listed below:

| | | |
|---|---|---|
| celA | Enterococcus gilvus | WP_10781765.1 |
| celB | Enterococcus gilvus | WP_010780456.1 |
| celC | Enterococcus gilvus | WP_010780458.1 |
| celA | Lactococcus lactis subsp. lactis | NP_266573.1 |
| celB | Lactococcus lactis subsp. lactis | NP_266330.1 |
| ptcA | Lactococcus lactis subsp. lactis | NP_266570.1 |
| celB | Bacillus coagulans | BF29_RS14550 |

A 6-phospho-beta-glucosidase (EC:3.2.1.86) is then required to convert the cellobiose-6P into one molecule of glucose and one molecule of glucose-6-P, both of which are readily used by the host. An example is the 6-phospho-beta-glucosidase from Bacillus coagulans, which has successfully been expressed in E. coli (Zheng et al. (2018) Biotechnology for Biofuels 18:320). Alternate candidates are listed below:

| | | |
|---|---|---|
| celA | Enterococcus gilvus | WP 0781765.1 |
| celB | Enterococcus gilvus | WP_010780456.1 |
| celC | Enterococcus gilvus | WP_010780458.1 |
| celA | Lactococcus lactis subsp. lactis | NP_266573.1 |
| celB | Lactococcus lactis subsp. lactis | NP 266330.1 |
| ptcA | Lactococcus lactis subsp. lactis | NP_266570.1 |
| celB | Bacillus coagulans | BF29_RS14550 |

In another embodiment, a microbe used to practice embodiments as provided herein is genetically modified by deleting or reducing expression of genes to eliminate or reduce production of metabolites, such as the polyamines putrescine, spermidine, and cadaverine. These molecules are essential for gastrointestinal mucosal cell growth and function, but excess of these compounds has been linked to gut dysbiosis and poor nutrient absorption (Forget et al. (1997) J. Pediatr. Gastroenterol. Nutr. 24:285-288). The primary routes for polyamine synthesis in bacteria are decarboxylation of the amino acid's arginine or ornithine. Ornithine decarboxylase (ODC, EC:4.1.1.17) converts ornithine to putrescine, while arginine decarboxylase (ADC, EC:4.1.1.19) converts arginine to agmatine, which is subsequently converted to putrescine by agmatinase (EC:3.5.3.11). Putrescine can then be converted to other derivatives such as spermidine. Therefore, a reduction in ODC and/or ADC expression will reduce polyamine production in the host microbe. E. coli contains two ODC isomers, encoded by the speC and speF genes, as well as two isomers of ADC encoded by speA and adiA. BLAST searches using these sequences, or other known bacterial ODC and ADC genes, applied to the genome of the organism of interest is used to identify genes encoding these functions in the organism to be genetically modified. One or both of these genes, or homologs thereof, are then deleted from the host genome using tools such as lambda-red mediated recombination (Datsenko and Wanner (2000) Proc. Nat. Acad. Sci. USA 97:6640-6645), CRISPR-Cas9 genome editing (Bruder et al (2016) Appl. Environ. Microbiol. 82:6109-6119), or any other method resulting in the removal of genes or portions of genes from the chromosome. In another embodiment, these methods are used to replace the native promoters of these genes with alternate promoters of different strengths, or to modify the ribosome binding site, resulting in reduced production of the ODC and ADC enzymes. In yet another embodiment, expression is reduced through a gene silencing mechanism such as antisense RNA-based attenuation (Nakashima et al. (2012) Methods Mol. Biol. 815:307-319) or CRISPR interference (Choudhary et al. (2015) Nat. Comm. 6:6267).

### Bioinformatic Discovery of Putative Immunomodulatory Proteins and Genetic Modification of Exemplary Therapeutic Microbes

In alternative embodiments, genetically modified microorganisms as provided herein, including microorganisms as listed in Tables 1, 4 and 7, and a bacterium from a combination of microbes as provided herein, for example, as in Table 9 and/or Table 42, Example 25, are engineered to express immunomodulatory, for example, immunostimulatory, proteins, or to overexpress endogenous immunomodulatory proteins. In alternative embodiments, the immunomodulatory are secreted or are cell surface-expressed or membrane-expressed proteins.

Organisms of interest are bioinformatically interrogated for expression of putative immunomodulatory proteins. Based on immune correlation analysis and the differential relative abundance of organisms between cancer and control samples, certain organisms are identified as being missing from the cancer microbiome and potentially immunostimulatory and having anti-cancer properties. These identified organisms can be incorporated into formulations as provided herein, or into combinations of microbes as provided herein; or, the immunomodulatory proteins they express are identified and genetically engineered into organisms as provided herein, for example, as listed in Tables 1, 4, 7, 8 and 9. In alternative embodiments, an organism as provided herein (as used in a method as provided herein) is genetically modified to overexpress the discovered immunomodulatory protein or proteins. Organisms potentially immunostimulatory and having anti-cancer properties are highlighted in Example 10.

For example, Dorea formicigenerans is one such organism, with strong positive correlations in both cancer and control cohorts to CD3+ and CD3+CD56+ immune cells in peripheral blood. First, a database of proteins is downloaded and clustered by similarity. Predicted proteins are downloaded from the NCBI RefSeq genomic database for a representative set of microbial genome assemblies. All complete genome assemblies for bacteria and archaea are included. For the taxa of special interest, which include Verrucomicrobia, Clostridia, and Coriobacteria, all assemblies of any status are included. Predicted proteins are downloaded from RefSeq and clustered using mmseqs2 (Steinegger and Soding. (2017) Nature Biotechnology 35:1026-1028). The resulting clusters contain proteins with identical or highly similar sequences. For a specific organism of interest, the protein clustering analysis is used to identify genes that are mostly unique to the organism yet ubiquitous across the organism's pangenome. These genes are likely candidates to mediate the immunomodulatory functions that are specific to the organism of interest. A standard bioinformatic analysis is performed on genes unique to the organism of interest to identify protein domains within each gene as being signal, cytoplasmic, non-cytoplasmic, or transmembrane domains. Because immunomodulatory genes need to interact with immune cells, they are generally secreted proteins (Quevrain et al. (2016) Gut 65:415-425) or membrane proteins (Plovier et al. (2017) Nature Medicine 23:107-113). Secreted proteins are identified from the analysis using the signal domains, while membrane proteins are identified by the presence of transmembrane domains. Because proteins with several transmembrane domains tend to be transporters, the focus is on proteins with one or two transmembrane domains. Membrane proteins or secreted proteins from the analysis of genes unique to the organism are prioritized for overexpression in genetically modified microorganisms as provided herein.

In alternative embodiments, genetically modified microorganisms as provided herein are engineered to express exogenous membrane proteins or secreted proteins. Genes unique to the organism of interest that are also membrane proteins or secreted proteins are investigated in a bespoke manner using the publicly available BLAST or Pfam search engines. In one embodiment, the organism is genetically modified to express these or homologues of identified membrane proteins. From this analysis, one protein from Dorea formicigenerans, NCBI Reference Sequence WP_118145075.1 is a particularly attractive candidate. The protein family for WP_118145075.1 contains 28 protein sequences, of which 26 are from Dorea formicigenerans genomes. There are 27 total Dorea formicigenerans assemblies in the database, so 26 out of 27 assemblies contains a version of protein WP_118145075.1. When analyzed on BLAST and Pfam, WP_118145075.1 is identified as a pilus-like protein. Pili and related proteins have a known role in interaction with human cells (Lizano et al. (2007) Journal of Bacteriology 189:1426-1434; Plovier et al. (2017) Nature Medicine 23:107-113; Ottman, N., et al. (2017) PLOS ONE 12(3):e0173004). Genes may also be identified as containing pilus-like structures or other known immunomodulatory structures using public available techniques such as PilFind (Imam et al. (2011) PLOS ONE 6(12):e28919). In alternative embodiments, these pilus-like structures or other known immunomodulatory structures are engineered into genetically modified microorganisms as provided herein.

Other pili-like proteins of interest and corresponding homologs used in genetically engineered organism as provided herein include the highly abundant outer membrane protein of Akkermansia muciniphila, Amuc_1100 and members of the Amuc_1098 Amuc_102 gene cluster, have been shown to induce the production of specific cytokines (IL-8, IL-1β, IL-6, IL-10 and TNF-α) through activation of Toll-like receptors (TLR) 2 and TLR4 (Ottman et al (2017) PLoS One 12, e0173004). Similar outer membrane proteins are believed to be responsible for the induction of cytokine IL-10 by commensal gut microbes such as Faecalibacterium prausnitzii A2-165 and Lactobacillus plantarum WCFS1.

In another embodiment, a genetically engineered organism as provided herein is genetically modified to express homologues of bacterial flagellin to induce TLR5 signaling. TLR5 response to flagellin promotes both innate and adaptive immune functions for gut homeostasis (Leifer et al (2014) Immunol. Lett. 162, 3-9). Recently, flagellin been examined for anti-tumor and radioprotective properties and has shown potential in reducing tumor growth and radiation-associated tissue damage (Hajam et al (2017) Exp. Mol. Med. 49, e373-e373). Some flagellin-based anti-tumor vaccines have also successfully entered into human clinical trials. Flagellins (fliC) and homologues of interest include but are not limited to those from Salmonella Typhimurium (FliCi), Escherichia coli, Pseudomonas aeruginosa, Listeria monocytogenes, and Serratia marcescens.

### Identification of Immunomodulatory Proteins Via Pooled Screening

In alternative embodiments, microbes used in compositions as provided herein, or as used in methods as provided herein, have enhanced immunomodulatory effects, for example, immune-stimulatory effects, and these microbes can be generated or derived either by selection using assays, as described below, or by inserting or enhancing the microbe's immunomodulatory effects by genetic engineering, for example, by inserting a heterologous nucleic acid into the microbe. In alternative embodiments, microbes that can express or overexpress immunomodulatory proteins or peptides are used with (in addition to, are administered with) microbes used in compositions as provided herein, or microbes used to practice methods as provided herein.

Microbial populations are assayed directly for immunomodulatory effects on dendritic cells. Starting with a fecal sample of interest containing an endogenous microbial population or starting with a synthetic microbial population consisting of pooled microbial isolates of interest, the population can be tested against dendritic cells ex vivo.

Purified dendritic cells are generated as described in previous work (Svensson and Wick. (1999) European Journal of Immunology 29(1):180-188; Svensson et al. (1997) Journal of Immunology 158(9):4229-4236; Yrlid et al. (2001) Infection and Immunity 69(9):5726-5735). Heat-inactivated, incubated for 30 minutes at 70° C., or live bacteria are added at a 50:1 ratio and incubated for 4 hours at 37° C. in IMDM containing 5% FBS. Following incubation, cells are washed 3× in HBSS to remove excess antigen. A portion of the dendritic cells are saved in RNAlater for future RNA sequencing. When activated, dendritic cells express several co-stimulatory molecules that aid in activating T cells. These molecules (CD40, CD80, and CD86) are upregulated alongside the chemokine receptor CCR7 which homes the activated DC to the spleen or local lymph node (Wilson and O'Neill. (2003) Blood 102(5):1661-1669; Ohl et al. (2004) Immunity 21(2):279-288). This set of genes can therefore be used to sort mature, activated DCs from immature DCs that do not stimulate T cells effectively. Cells are stained for expression of one or more of CD86, CD40 and CD80, and sorted via Fluorescence Activated Cell Sorting (FACS).

Purified cells are processed as described previously (Abelin et al. (2017) Immunity 46(2):315-326) for HLA-peptide identification. Briefly, purified cells are dissociated in protein lysis buffer containing protease inhibitors and DNAse, and then sonicated. Following sonication, soluble lysates are incubated with SEPHAROSE™ beads linked to W6/32 antibody which are washed with lysis buffer lacking protease inhibitor, and finally washed with DI water. Peptides are then eluted from the HLA complex on EMPORE C18 STAGETIPS™. Purified protein preparations are then subjected to nanoLC-ESI-MS/MS.

Following LC-MS/MS, individual peptides are identified and matched to the reference genomes of the mix of microbes used in the in vitro activation experiment. A list of candidate peptides is generated by combining peptide abundance data with bioinformatics analysis of protein conservation, localization data, and their likelihood to express and localize to the membrane (Marshall et al. (2016) Cell Reports 16(8):2169-2177; Saladi et al. (2018) Journal of Biological Chemistry 293(13):4913-4927).

Identification and Validation of Microbes that Activate Immune Cell Receptors

In alternative embodiments, microbes used in compositions as provided herein, or as used in methods as provided herein, can activate immune cell receptors (for example, such as T cell receptors), and these microbes can be generated or derived either by selection using assays, as described below, or by inserting or enhancing the microbe's immunomodulatory effects by genetic engineering, for example, by inserting a heterologous nucleic acid into the microbe. In alternative embodiments, microbes that can express or overexpress proteins or peptides that can activate immune cell receptors are used with (in addition to, are administered with) microbes used in compositions as provided herein, or microbes used to practice methods as provided herein.

In alternative embodiments, ex vivo analyses are used to identify microbes that activate immune cell receptors including but not limited to dendritic cell Toll-like receptors (TLR's). Briefly, microbes of interest are co-incubated with human dendritic cells as described in the previous section, except that the co-incubation occurs with a pasteurized and washed microbial isolate rather than a microbial population. Dendritic cells are washed post-incubation. As described in Example 11, dendritic cells are saved and analyzed using RNA sequencing to identify gene expression changes relative to control conditions. The control conditions include both no stimulation i.e. microbial media alone, as well as known agonists for different TLR's. A computational analysis is performed to ascribe the gene expression of dendritic cells in response to each microbe to some amount of activation of each TLR, thus predicting microbe-TLR interactions.

For each predicted microbe-TLR interaction, the pasteurized and washed microbe is co-incubated with TLR reporter cells (HEK-Blue, InvivoGen), and a plate-based colorimetric assay used to measure TLR activation over time. Validated microbes can be further screened as described previously for specific genes that mediate their mechanistic effects.

### Amplification of Therapeutic Effect by Overexpression of Immunomodulatory Genes

In alternative embodiments, microbes used in compositions as provided herein, or as used in methods as provided herein, overexpress immunomodulatory genes (for example, immunostimulatory genes), and these microbes can be generated or derived either by selection using assays, as described below, or by inserting or enhancing the microbe's immunomodulatory effects by genetic engineering, for example, by inserting a heterologous nucleic acid into the microbe.

In alternative embodiments, microbes used in compositions as provided herein, or used to practice methods as provided herein, are selected to express, or overexpress, an anti-viral molecule (such as an anti-viral small molecule, peptide or polypeptide such as an anti-viral antibody; or an anti-viral drug as provided or as described herein), an immunostimulatory protein or peptide, which can be non-specific immunostimulatory proteins such as a cytokine, for example, a cytokine such as an interferon (for example, IFN-α2a, IFN-α2b) IL-2, IL-4, IL-6, IL-7, IL-12, IFNs, TNF-α, granulocyte colony-stimulating factor (G-CSF, also known as filgrastim, lenograstim or NEUPOGEN®) and granulocyte monocyte colony-stimulating factor (GM-CSF, also known as molgramostim, sargramostim, LEUKOMAX®, MIELOGEN® or LEUKINE®), or a specific immunostimulatory protein or peptide, for example, such as an immunogen that can generate a specific humeral or cellular immune response, for example, an immune response to a viral antigen. In alternative embodiments, microbes that can express or overexpress immunostimulatory proteins or peptides are used with (in addition to, are administered with) microbes used in compositions as provided herein or used to practice methods as provided herein.

In one embodiment, an organism used to practice embodiments as provided herein is genetically modified to overexpress proteins or peptides with antiviral properties or as immunogenic components of antiviral vaccines. Several peptides have been identified that have the potential to interfere with the course of infection by the SARS-CoV2 virus (Mahendran et al (2020) Frontiers in Pharmacology https://doi.org/10.3389/fphar.2020.578382). For instance, the 36-mer peptide EK1 binds to the HR1 domain of the SARS-CoV2 spike protein, thereby inhibiting viral fusion entry into target host cells (Xia et al (2019) Cell. and Molec. Immunol. 17:765 https://doi.org/10.1038/s41423-020-0374-2). In another example, the 17-mer peptide Mucroporin-M1 is a mutational variant of an active protein from the venom of the scorpion Lychas mucronatus that is optimized for insertion and disruption of viral lipid envelopes such as that of SARS-CoV1 (Li et al (2011) Peptides 32:1518 DOI: 10.1016/j.peptides.2011.05.015). In another example, a 30-mer peptide derived from mouse b-defensins binds to the S2 subunit of MERS-CoV viral particles and upon fusion-entry blocks further viral infection progression by preventing acidification of the endosome (Zhao et al (2016) Scientific Reports https://doi.org/10.1038/srep22008). In other examples, peptides or proteins can be produced by overexpression in bacterial scaffold expression hosts that can serve as immunogenic components of peptide-based antiviral vaccines (Di Natale et al (2020) Frontiers in Pharmacology https://doi.org/10.3389/fphar.2020.578382). For instance, the anti-COVID vaccine NVX-CoV2373 in development by the company Novavax is a protein subunit nanoparticle vaccine comprised of expressed SARS-CoV2 Spike Protein and Matrix-M1 protein (Keech et al (2020) New Eng. J. Med. 383:2320 DOI: 10.1056/NEJMoa2026920). In another example, bacterial expression chassis can serve to both express vaccine protein components as well as a delivery vehicle of such vaccine components to the gut mucosa (Thole et al (2000) Current Opinion in Molec. Therapeutics 2:94 PMID: 11249657), where it can elicit immune responses against gut-localized infection by SARS-CoV2 or other gastrointestinal viruses.

Genes identified from a bioinformatic or pooled experimental approach as having an immunomodulatory effect are validated using recombinant expression in an engineered chassis organism. In alternative embodiments, the engineered chassis organism is used as a strong modulator (for example, stimulator) of immune activity as a component of a live biotherapeutic as provided herein (for example, as a component of a combination of microbes as provided herein, for example, as a component of an exemplary combination as listed in Table 9 and/or Table 42, Example 25), or the engineered chassis organism can be used in addition to a live biotherapeutic as provided herein.

Nucleic acids encoding protein sequences capable of enhancing a microbe's immunomodulatory effects are synthesized and cloned or inserted into a microbe, for example, bacterium, used in a combination of microbes as provided herein (as in for example, Table 9 and/or Table 42, Example 25), including for example any bacterium as listed in Table 1, Table 4 or Table 7, for example, such as a B. subtilis. B. subtilis is a generally recognized as safe (GRAS) organism that has extensive tools available for the cloning and expression of synthetically encoded proteins (see for example, Popp et al. (2017) Scientific Reports 7(1):15058). Following cloning, colonies containing each different synthetic protein are grown until logarithmic phase. Each culture is pasteurized and washed as described previously. The cultures are validated for immunomodulatory activity relative to a negative control consisting of the unmodified chassis organism and positive control consisting of the unmodified original microbe of interest.

Each overexpressed gene can be validated for immunomodulatory activity using a TLR reporter assay as described previously, or a co-incubation with dendritic cells followed by mass spectrometry or RNA sequencing as described previously. Validated immunomodulatory engineered microbes can be incorporated into the candidate live biotherapeutic and advanced to in vivo screening in animal models.

### Example 13: Whole Cell Mutagenesis and Selection Procedures for Therapeutic Microbes

In alternative embodiments, microbes as provided herein (including bacteria from all the genuses listed herein), and including the combinations of microbes as provided herein, for example, the exemplary combinations of microbes 1 to 294 as described in Table 9, Example 10, and/or Table 42, Example 25, are genetically modified to enhance antiviral capability, for example, increase the ability of the immune system to combat viral infections, stimulate activity of specific classes of immune cells, provide essential nutrients that may be depleted or blocked by the virus, produce compounds with antiviral activity, or other direct or indirect effect on cells of the innate or adaptive immune system.

Candidate live biotherapeutic strains are randomly mutagenized to generate a microbe with increased level of production of either a protein or metabolite of interest that may impact cancer treatment. When cells are mutagenized, changes occur in the DNA sequence that could result in changes of expression levels of certain genes. Often these mutations are lethal, but some strains survive and have altered phenotype, including some with increased expression of genes encoding proteins or metabolic pathways identified from patient data (Examples 9 and 10) or in vitro assays (Example 11). Mutagenesis is carried out by an established treatment such as ultraviolet light, N-ethyl-N-nitrosourea, or ethyl methanesulfonate, followed by culturing on non-selective media to obtain viable cells. Mutagen exposure is first tuned by varying the time or intensity of treatment to a small culture, then selecting the conditions which yield approximately 10-20% of the number of viable colonies compared to a non-treated control. These treatment conditions are then applied to a larger culture of cells, and mutagenized colonies obtained are screened for the phenotype of interest, such as increased production of a protein or metabolite of interest. Clones obtained from this screen are then further characterized by whole genome sequencing.

### Example 14: Production of Live Biotherapeutics

In alternative embodiments, microbes as provided herein (including bacteria from all the genuses listed herein), and including the combinations of microbes as provided herein, for example, the exemplary combinations 1 to 294 as described in Table 9, Example 10, and/or Table 42, Example 25, comprise anaerobic bacteria, including anaerobic bacteria isolated from a fecal sample, cultured anaerobic bacteria, or a combination thereof.

### Individual Culture of Anaerobic Microbes for Mouse Studies

Anaerobic microbes of interest are cultured in multiples of 1-liter volumes in anaerobic media bottles as follows. Microbes in cryostorage are plated and struck on appropriate anaerobic solid medium and then cultured at 37° C. to obtain isolated colonies. For each microbe, a single colony is inoculated into a Hungate tube containing 10 ml appropriate anaerobic growth medium and allowed to grow at 37° C. until turbid to create a starter culture. For each microbe of interest, multiple 0.9-liter volumes of appropriate liquid anaerobic medium in 1 L anaerobic bottles (as described in Example 1) are inoculated with 2 ml starter culture each using a needle and syringe. The number of 1-liter cultures for each microbe is dependent on the necessary final amount of live cell mass for formulation into live biotherapeutics for mouse studies. Inoculated bottles are placed upright on a platform shaker at 115 rpm at 37° C. for 48 hours or until growth turbidity is evident. Growth density is monitored by taking 1 ml samples during the cultures for optical density measurements at 600 nm. Optical densities of 1.0 to 4.0 can be obtained after 48 hours depending on the microbe cultured. Prior to large scale culture, cell densities are determined empirically for each microbe by dilution plating and colony counting to determine the colony forming units (CFU) per ml at an optical density of 1.0.

Large scale cultures are grown to attain a final live density of 1.0E8 to 1.0E9 CFU/ml, and then the culture bottles are brought into the anaerobic chamber for harvesting of live cell mass. Once in the chamber, the aluminum collars and butyl rubber bungs are removed, and the 1-liter contents of each culture bottle are poured into two 500 ml centrifuge bottles with rubber gasketed screw caps. After decanting growth medium, the caps of the centrifuge bottles are tightened for an airtight seal, brought out of the anaerobic chamber, then centrifuged for 20 minutes at 6000 g at 4° C. Centrifuged bottles are then brought into the anaerobic chamber, uncapped, and then the supernatants are poured off and discarded. The remaining cell pellets are then combined with 250 ml ice cold Vehicle Buffer (Phosphate Buffered Saline plus 1 g/L L-cysteine plus 15% glycerol, filter sterilized and made anoxic by bubbling with filtered nitrogen). The cell pellets are carefully resuspended in the Vehicle Buffer on ice; the resuspended volumes of two pellets are combined into one 500 ml bottle, recapped for an air-tight seal, removed from the anaerobic chamber, then centrifuged for 20 minutes at 6000 g at 4° C. After decanting supernatants in the anaerobic chamber, resulting cell pellets are then carefully resuspended once more with 250 ml ice cold Vehicle Buffer in the anaerobic chamber, removed from the anaerobic chamber, then centrifuged for 20 minutes at 6000 g at 4° C. After removal of supernatant in the anaerobic chamber, each pellet is resuspended in 100 ml ice cold Vehicle Buffer to establish a ten-fold concentration of the original culture cell density.

Within the anaerobic chamber, final resuspended cell pellet volumes for an anaerobic microbe of interest are combined and thoroughly mixed in a sterile bottle by gentle stirring on a stir plate on ice. The volume is then dispensed into 25 ml aliquots in 50 ml conical tubes using a serological pipette, then a stream of sterile filtered gaseous argon is introduced to each tube to displace the headspace and to serve as an oxygen barrier. Each tube is then tightly capped, and the seal is wrapped with several layers of parafilm. The tubes are then racked upright, removed from the anaerobic chamber, and then allowed to slowly freeze at −80° C. A smaller 5 ml aliquot is also made for each preparation and stored as described above. After 18 hours, the 5 ml aliquots for each microbial strain of interest are removed and allowed to thaw standing in ice water within the anaerobic chamber. The thawed volumes are gently mixed by inversion several times, then subjected to dilution plating on appropriate solid anaerobic medium to determine the live cell density in CFU/ml after freezer storage.

### Live Biotherapeutic Assembly for Mouse Studies

Live biotherapeutic compositions of anaerobic microbes of interest, including the combinations of microbes as provided herein, for example, the exemplary combinations 1 to 294 as described in Table 9, Example 10, and/or Table 42, Example 25, are assembled in volumes that are pertinent for projected mouse studies. Enough aliquots for each microbe of interest are removed from storage at −80° C. and gently thawed in ice water in the anaerobic chamber. The thawed multiple aliquots are combined in a sterile bottle, gently remixed and then placed on ice. The amount of volume of each microbe to add to a mix is adjusted so that the determined live cell densities for each microbe are equivalent, and final total cell densities can be adjusted by further addition of ice-cold vehicle buffer. Once all requisite volumes for each microbe are added together in a larger sterile bottle, the volume is gently mixed by stirring on a stir plate on ice.

Live biotherapeutic volumes are then re-aliquoted in individual volumes that each comprise a projected daily dose of live microbes in anticipated mouse studies. Determined volumes are each dispensed in 15 ml conical tubes up to 10 ml per aliquot. The volume in each tube is overlaid with a stream of sterile filtered argon to displace oxygen, followed by capping. Live biotherapeutic aliquot tubes are racked upright and allowed to slowly freeze at −80° C. After 48 hours, one aliquot for each microbial mix preparation is thawed and dilution plated to validate the final total CFU/ml, optimally at greater than 1.0×10⁹ CFU/ml.

### Example 15—Efficacy of Anticancer Live Biotherapeutics with Checkpoint Inhibitors

The results described here were obtained from studies conducted with tumor mouse models evaluating the anticancer efficacy of generated live biotherapeutics as a monotherapy and in combination with checkpoint therapy. Microbes, gene functions, and metabolites elucidated as critical for anticancer treatment are relevant for the treatment of viral infections because in both cases a healthy immune response is required to combat the disease. Therefore, it is reasonable to expect that microbes beneficial for immuno-oncology treatment will also be beneficial or even essential for rapid viral clearance.

Mice with and without tumors are given microbial cocktails by oral gavage, as described in the example above. The 16S RNA sequencing results are used to determine the distribution of organisms in each sample at both the phylum and genus level, and the distribution is compared across all fecal samples from mice without tumors to determine how these microbes colonize the gut. PCA is used to classify all samples of mice without tumors, showing that samples with the same microbial treatment type cluster together. In addition, the genera represented by each microbial treatment have increased representation in those samples compared to those of different treatment type.

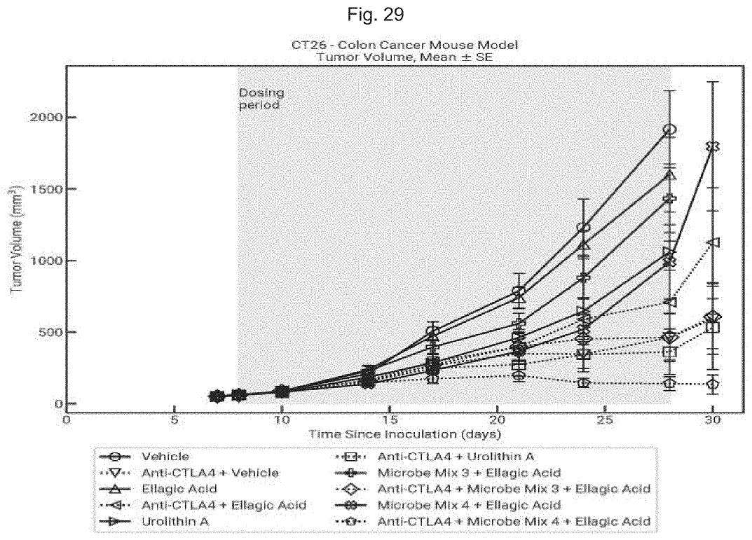

Tumor size is measured in all animals receiving the different microbial treatments, with and without anti-CTLA4 therapy. On average, the animals receiving microbial mix 4 (equal amounts of F. prausnitzii, C. coccoides, R. gnavus, C. scindens, E. lenta, and G. urolithinfaciens) in conjunction with ellagic acid and anti-CTLA4 have a reduction in tumor size compared to those with other microbes or not receiving any anti-CTLA-4 treatment, as illustrated in FIG. 3. Termination of dosing of both the microbial and anti-CTLA-4 treatments were performed at day 28 and mice were evaluated. Mice treated with microbial mix 4 and the anti-CTLA-4 therapy had minimal tumor growth in contrast to the other groups, as shown in FIG. 29.

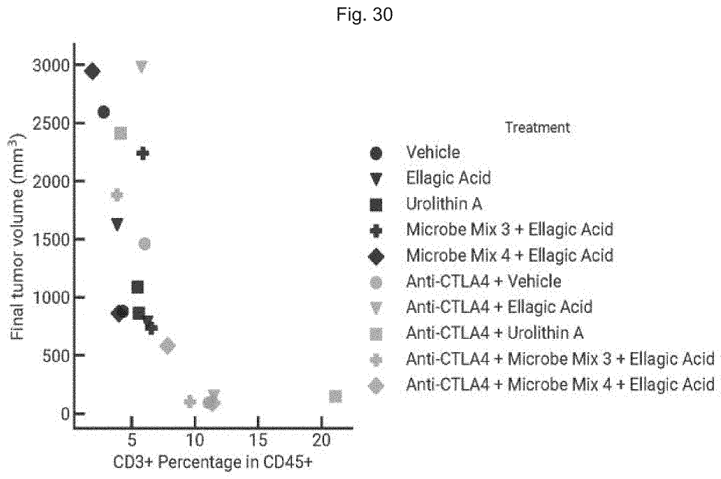

FACS analysis of whole blood obtained from the animals at the end of the study indicated that CD4 and CD8 T-lymphocyte activity are increased by treatment with microbial mix 4 in conjunction with anti-CTLA-4 as shown in the “population table” of FIG. 30. Similarly, it has been shown in mice that the commensal microbiota critically regulates the generation of virus-specific CD4 and CD8 T cells and antibody responses following respiratory influenza virus infection (T. Ichinohe et al., Proc. Natl. Acad. Sci. U.S.A 108, 5354-9 (2011)). This further supports that live biotherapeutics described herein that are critical for immuno-oncology treatment will also be beneficial or even essential for rapid viral clearance.

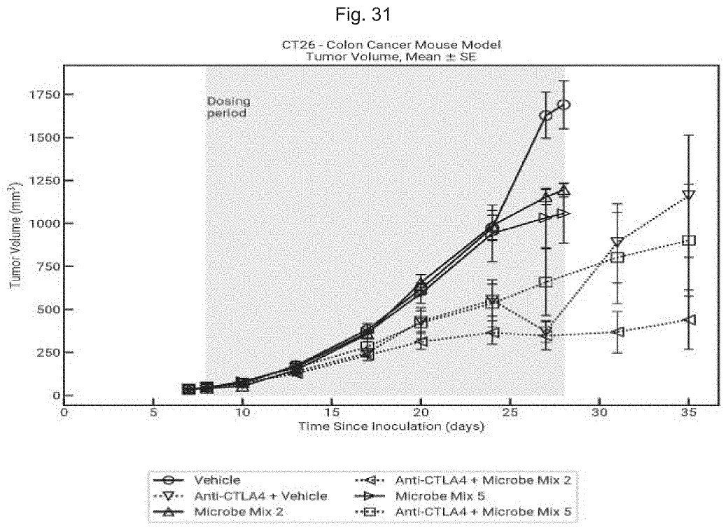

FIG. 31 graphically illustrates data showing the efficacy of anti-CTLA-4 treatment in mice with CT26 cancer tumor graft and supplemented with nutrients and/or microbial mixtures. Datapoints refer to tumor volume (mm³) at each day measurements were taken, averaged over either 8 mice (no CTLA-4) or 8 mice (with CTLA-4) with standard error shown.

Tumor size is measured in all animals receiving the different microbial treatments, with and without anti-CTLA-4 therapy. On average, the animals receiving microbial mix 2 (F. prausnitzii, C. coccoides, R. gnavus, C. scindens, A. muciniphila, and E. hirae) in conjunction with anti-CTLA-4 have a reduction in tumor size compared to those with other microbes or not receiving any anti-CTLA-4 treatment, as illustrated in FIG. 31.

### Metabolomics

Commensal microbiota metabolites have been shown to be critical in suppressing influenza virus as well as the replication of herpes simplex virus (HSV)-2 (N. Li, et. al. Front. Immunol. 10 (2019), p. 1551). The results described here were obtained from studies conducted with tumor mouse models evaluating the anticancer efficacy of generated live biotherapeutics as a monotherapy and in combination with checkpoint therapy. Metabolites elucidated as critical for anticancer treatment are relevant for the treatment of viral infections because in both cases a healthy immune response is required to combat the disease. Therefore, it is reasonable to expect that microbial metabolites beneficial for rapid viral clearance.

Mouse fecal samples, either raw or resuspended in PBS, were kept frozen at −80 degrees C. until processing, then immediately placed in a lyophilizer and freeze-dried overnight. The resulting material was weighed, and lyophilized fecal samples were extracted and processed at a constant per-mass basis using an established procedure (Evans, A. et al. High resolution mass spectrometry improves data quantity and quality as compared to unit mass resolution mass spectrometry in high-throughput profiling metabolomics. J. Postgenomics Drug Biomark. Dev. 4, S24-S36 (2014)) by Metabolon, Inc. Recovery standards were added before the first step in the extraction process for quality-control purposes. Samples are prepared using the automated MicroLab STAR® system from Hamilton Company. Several recovery standards are added prior to the first step in the extraction process for QC purposes. Samples are extracted with methanol under vigorous shaking for 2 min (Glen Mills GenoGrinder 2000) to precipitate protein and dissociate small molecules bound to protein or trapped in the precipitated protein matrix, followed by centrifugation to recover chemically diverse metabolites. The resulting extract is divided into five fractions: two for analysis by two separate reverse phase (RP)/UPLC-MS/MS methods using positive ion mode electrospray ionization (ESI), one for analysis by RP/UPLC-MS/MS using negative ion mode ESI, one for analysis by HILIC/UPLC-MS/MS using negative ion mode ESI, and one reserved for backup. Samples are placed briefly on a TurboVap® (Zymark) to remove the organic solvent. The sample extracts are stored overnight under nitrogen before preparation for analysis.

All analytical methods utilize a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract is dried then reconstituted in solvents compatible to each of the four methods. Each reconstitution solvent contains a series of standards at fixed concentrations to ensure injection and chromatographic consistency. One aliquot is analyzed using acidic positive ion conditions, chromatographically optimized for more hydrophilic compounds. In this method, the extract is gradient-eluted from a C18 column (Waters UPLC BEH C18-2.1×100 mm, 1.7 μm) using water and methanol, containing 0.05% perfluoropentanoic acid (PFPA) and 0.1% formic acid (FA). A second aliquot is also analyzed using acidic positive ion conditions but is chromatographically optimized for more hydrophobic compounds. In this method, the extract is gradient eluted from the aforementioned C18 column using methanol, acetonitrile, water, 0.05% PFPA and 0.01% FA, and is operated at an overall higher organic content. A third aliquot is analyzed using basic negative ion optimized conditions using a separate dedicated C18 column. The basic extracts are gradient eluted from the column using methanol and water, however with 6.5 mM Ammonium Bicarbonate at pH 8. The fourth aliquot is analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis alternates between MS and data-dependent MSⁿ scans using dynamic exclusion. The scan range varies slightly between methods, but covers approximately 70-1000 m/z.

Three types of controls were analyzed in concert with the experimental samples: a pooled sample generated from a small portion of each experimental sample of interest served as a technical replicate throughout the platform run; extracted water samples served as process blanks; and a cocktail of standards spiked into every analyzed sample allowed for instrument performance monitoring. Instrument variability was determined by calculation of the median relative s.d. (RSD) for the standards that were added to each sample before injection into the mass spectrometers (median RSDs were determined to be 3%). Overall process variability was determined by calculating the median RSD for all endogenous metabolites (i.e., non-instrument standards) present in 90% or more of the pooled technical-replicate samples (median RSD=8%, n=797 metabolites).

Compounds are identified by comparison to library entries of purified standards maintained by Metabolon, that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library+/−10 ppm, and the MS/MS forward and reverse scores. MS/MS scores are based on a comparison of the ions present in the experimental spectrum to ions present in the library entry spectrum. While there may be similarities between these molecules based on one of these factors, the use of all three data points can be utilized to distinguish and differentiate biochemicals. Peaks are quantified as area-under-the-curve detector ion counts.

### Metabolomics Performed on Fecal Samples

Metabolomics was performed on fecal samples taken from mice in the control group, treated with vehicle and no checkpoint inhibitor, the group treated with microbial mix 4 and ellagic acid only, the group treated with anti-CTLA-4 only, and the group treated with anti-CTLA-4, microbial mix 4, and ellagic acid. In the tables and figures that follow, these are referred to as the Control, Microbe, Drug, and Combo, respectively. Samples were processed from timepoint 1 (T1), prior to any treatment; timepoint 4 (T4), 10 days from start and 48 hours after the 3^{rd} treatment dose; and timepoint 7 (T7), 20 days from start and 48 hours after the 6^{th} treatment dose.

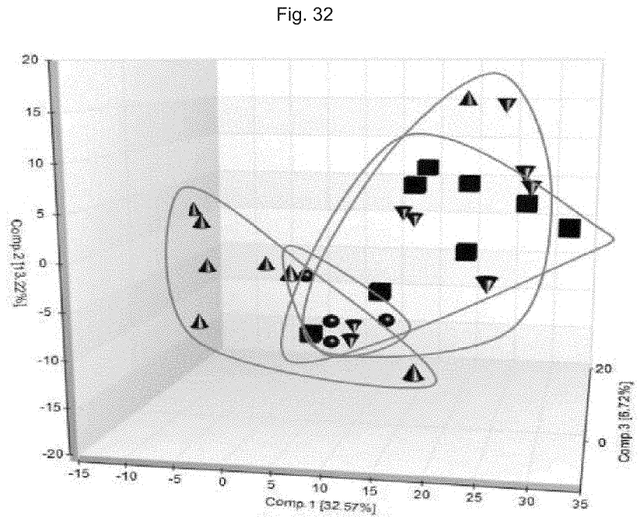

Principal components analysis (PCA) was applied on all samples to give a global view of the data. The Control group segregated by timepoint, indicating a gradual shift in the metabolome over time as the cancer progressed. A similar pattern was exhibited by the drug group, while the Microbe and Combo groups shifted in a different direction. There was little distinction among treatment groups at T1 and T4, while significant differences were observed at T7 (FIG. 32). At T7, the microbe and combo groups had changes with p<0.05 in 25% and 40% of all the metabolites detected, respectively, whereas the drug group only had such change in 9% of the metabolites.

Next, individual metabolic pathways and classes of metabolites were considered. The levels of amino acids (unmodified, gamma-glutamyl and acetylated) along with peptides (dipeptides and polypeptides) were lower in the Microbe and Combo groups relative to the Controls at T7 (Table 20). Declines in dipeptides and amino acids in the fecal samples highlight the possibility that proteolysis of both human and microbial-derived peptides, and microbial amino acid excretion, may have lessened following treatment with microbial mix 4. More evidence to support this notion came from the levels of gamma-glutamyl amino acids and N-acetylated amino acids, both of which were decreased in the fecal samples of Microbe and Combo groups. N-acetyl amino acids can be derived from proteins that have undergone post-translational acetylation reactions or from free amino acids reacting with acetyl groups. Gamma-glutamyl AAs are generated by gamma-glutamyl transpeptidase, which plays an important role in amino acid uptake. Decreased fecal levels of proteolysis markers may reflect diminished gut motility and increased transit time.

TABLE 20
Amino acids, acylated amino acids, and gamma-glutamyl amino
acids in mouse fecal samples at T7. Ratio of the mean peak areas for the
specifie dmetabolites in each group relative to the control group. Up or down
arrows indicate whether the increase or decrease in the treatment relative to
the control is significant based on Welch’s two-sample t-test with p < 0.05.

| Compound | Microbe T7 | | Drug T7 | | Combo T7 | |
|---|---|---|---|---|---|---|
| Glycine | 0.59 | ↓ | 0.79 | | ↓ | 0.71 |
| Serine | 0.59 | ↓ | 0.81 | | ↓ | 0.60 |
| Threonine | 0.46 | ↓ | 0.84 | | ↓ | 0.57 |
| Alanine | 0.59 | ↓ | 0.97 | | ↓ | 0.65 |
| Aspartate | 0.49 | ↓ | 0.81 | | | 0.62 |
| Asparagine | 0.30 | ↓ | 0.59 | ↓ | ↓ | 0.42 |
| Glutamate | 0.51 | ↓ | 0.97 | | ↓ | 0.57 |
| glutamine | 0.91 | | 0.71 | | ↓ | 0.62 |
| histidine | 0.77 | | 0.84 | | ↓ | 0.62 |
| lysine | 0.50 | ↓ | 1.05 | | ↓ | 0.56 |
| Phenylalanine | 0.74 | | 0.98 | | ↓ | 0.66 |
| tyrosine | 0.60 | ↓ | 0.97 | | ↓ | 0.57 |
| tryptophan | 0.79 | | 0.91 | | ↓ | 0.67 |
| Leucine | 0.74 | | 0.97 | | ↓ | 0.66 |
| isoleucine | 0.61 | ↓ | 0.92 | | ↓ | 0.65 |
| valine | 0.60 | | 0.97 | | ↓ | 0.64 |
| Arginine | 0.95 | | 1.39 | | | 0.86 |
| proline | 1.14 | | 1.12 | | | 1.03 |
| N-acetylserine | 0.79 | | 1.42 | | | 0.53 |
| N-acetylthreonine | 0.59 | | 1 | | ↓ | 0.42 |
| N-acetylalanine | 0.47 | ↓ | 1.05 | | ↓ | 0.52 |
| N-acetylaspartate | 0.27 | ↓ | 1.04 | | ↓ | 0.65 |
| N-acetylasparagine | 0.27 | ↓ | 0.93 | | ↓ | 0.36 |
| N-acetylglutamate | 0.36 | ↓ | 1.18 | | ↓ | 0.76 |
| N-acetylglutamine | 0.86 | | 0.94 | | ↓ | 0.64 |
| N-acetylhistidine | 0.88 | | 0.88 | | | 0.67 |
| N2-acetyllysine | 0.37 | ↓ | 1.04 | | ↓ | 0.61 |
| N6-acetyllysine | 0.41 | ↓ | 1.05 | | ↓ | 0.56 |
| N-acetylphenylalanine | 0.71 | | 0.95 | | ↓ | 0.53 |
| N-acetyltyrosine | 0.38 | ↓ | 0.96 | | ↓ | 0.36 |
| N-acetyltryptophan | 1.17 | | 0.97 | | | 1.01 |
| N-acetylleucine | 0.78 | | 1.12 | | | 0.58 |
| N-acetylisoleucine | 0.79 | | 0.99 | | ↓ | 0.55 |
| N-acetylvaline | 0.99 | | 1.3 | | | 0.8 |
| N-acetylarginine | 0.59 | | 1.2 | | ↓ | 0.51 |
| N-acetylcitrulline | 0.4 | ↓ | 1.23 | | ↓ | 0.35 |
| N-acetylproline | 0.85 | | 0.97 | | ↓ | 0.66 |
| Gamma-glutamylglutamate | 0.48 | ↓ | 0.91 | | ↓ | 0.55 |
| Gamma-glutamylglutamine | 0.77 | | 0.69 | | ↓ | 0.56 |
| Gamma-glutamylisoleucine | 1.07 | | 0.79 | | | 1.65 |
| Gamma-glutamylleucine | 0.57 | | 0.83 | | ↓ | 0.50 |
| Gamma-glutamylalpha-lysine | 0.36 | ↓ | 0.69 | | ↓ | 0.36 |
| Gamma-glutamylepsilon-lysine | 0.48 | | 0.72 | | ↓ | 0.24 |
| Gamma-glutamylmethionine | 0.33 | ↓ | 0.86 | | ↓ | 0.41 |
| Gamma-glutamylphenylalanine | 0.61 | | 0.93 | | ↓ | 0.53 |
| Gamma-glutamylthreonine | 0.39 | ↓ | 0.82 | | ↓ | 0.58 |
| Gamma-glutamyltyrosine | 0.53 | | 0.98 | | ↓ | 0.48 |
| Gamma-glutamylvaline | 0.30 | | 0.89 | | | 0.55 |
| Gamma-glutamylserine | 0.47 | ↓ | 0.74 | | ↓ | 0.42 |
| Gamma-glutamylcitrulline | 0.25 | ↓ | 0.83 | | | 0.51 |

Cysteine is an important amino acid for redox balance because it contains a highly reactive thiol group which imparts the ability to participate in numerous reactions. Cysteine can be synthesized from methionine and serves as a precursor to antioxidants such as glutathione and taurine. Cysteine levels, as were upstream and downstream metabolites, were lower in the Microbe and Combo groups relative to Control (Table 21). This was consistent with the overall pattern of amino acid detection. Changes in cysteine metabolites may be signals of changes in redox status, as they are precursors for glutathione synthesis.

TABLE 21
Methionine and derivatives in mouse fecal samples at time T7.
Ratio of the mean peak areas for the specified metabolites in each
group relative to the control group. Up or down arrows indicate whether
the increase or decrease in the treatment relative to the control is
significant based on Welch’s two-sample t-test with p < 0.05.

| Compound | Microbe T7 | | Drug T7 | Combo T7 | |
|---|---|---|---|---|---|
| Methionine | 0.43 | ↓ | 0.95 | 0.5 | ↓ |
| N-acetylmethionine | 0.56 | ↓ | 0.99 | 0.58 | ↓ |
| N-formylmethionine | 0.64 | | 1.09 | 0.57 | ↓ |
| Methionine sulfoxide | 0.52 | ↓ | 0.93 | 0.6 | ↓ |
| N-acetylmethionine sulfoxide | 0.76 | | 0.88 | 0.64 | ↓ |
| cysteine | 0.59 | ↓ | 1 | 0.72 | ↓ |
| N-acetylcysteine | 0.44 | ↓ | 1.16 | 0.49 | ↓ |
| Cysteine sulfate | 0.87 | | 0.72 | 1.33 | |
| cystine | 0.39 | ↓ | 0.68 | 0.32 | ↓ |
| taurine | 1.19 | | 1.75 | 1.85 | |
| 3-sulfo-L-alanine | 0.3 | ↓ | 1.04 | 0.54 | ↓ |

Carboxyethyl amino acids were elevated only following Microbe monotherapy. Interestingly, this increase was not sustained during the combination treatment (Table 22). The Drug potentially had an opposing effect on the production of these analytes. Indeed, although never reaching significance, these levels tended to be lower in the Drug T7 group relative to Control.

TABLE 22
Carboxyethyl amino acids in mouse fecal samples at time T7. Ratio
of the mean peak areas for the specified metabolites in each group
relative to the control group. Up or down arrows indicate whether
the increase or decrease in the treatment relative to the control is
significant based on Welch’s two-sample t-test with p < 0.05.

| Compound | Microbe T7 | Drug T7 | Combo T7 |
|---|---|---|---|
| 1-carboxy ethylisoleucine | 1.56 | 0.64 | 0.89 |
| 1-carboxy ethylleucine | 2.43 | 0.67 | 0.82 |
| 1-carboxy ethylphenylalanine | 2.77 | 0.69 | 0.92 |
| 1-carboxy ethyltyrosine | 2.52 | 0.8 | 1 |
| 1-carboxy ethylvaline | 3.26 | 0.84 | 1.19 |

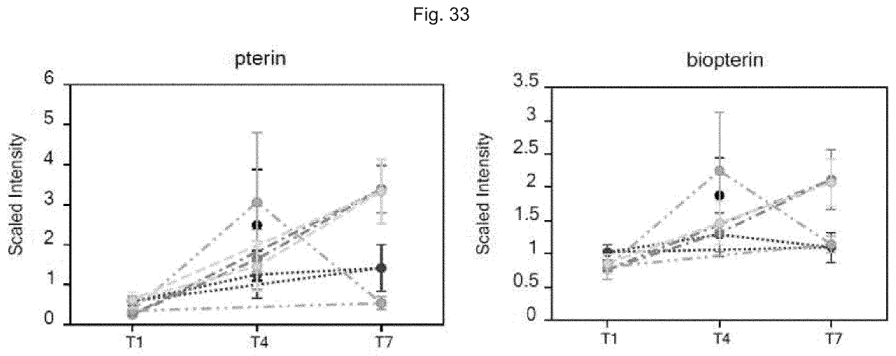

Pterins make up a group of small metabolites that serve as cofactors for various cell processes. Pterins are excreted by human urine and elevated levels have been detected when the cellular immune system is activated by diseases such as cancer (Koslinski, P., et al., Metabolic profiling of pteridines for determination of potential biomarkers in cancer diseases. Electrophoresis, 2011. 32(15): p. 2044-54). In humans, 5,6,7,8-tetrahydrobiopterin (BH4) is the most important unconjugated pterin and a cofactor for the hydroxylation of aromatic amino acids (phenylalanine, tyrosine, and tryptophan), the biosynthesis of the neurotransmitters serotonin and dopamine and the vasodilator nitric oxide (NO) (Thony, B., G. Auerbach, and N. Blau, Tetrahydrobiopterin biosynthesis, regeneration and functions. Biochem J, 2000. 347 Pt 1: p. 1-16), and for the biosynthesis of thymidine. Pterins may be host or bacterial-derived. BH4 is absorbed in the small intestine but in the colon, it is decomposed by enteric bacteria (Sawabe, K., et al., Tetrahydrobiopterin in intestinal lumen: its absorption and secretion in the small intestine and the elimination in the large intestine. J Inherit Metab Dis, 2009. 32(1): p. 79-85). Pterin and biopterin are BH4 degradation products. BH4 was not detected in these samples, but the degradation products increased over time in the Drug and Control group; however, levels were stationary in the Combo group and decreased after an initial rise in the Microbe group (see FIG. 33).

The polyamines, putrescine, spermidine and spermine, are organic polycations present in all eukaryotes and are essential for cell proliferation. Polyamines have been proposed to regulate cellular activities at transcriptional, translational and post-translational levels. The main sources for polyamines in mammals are cellular synthesis, food intake and microbial synthesis in the gut. The rate limiting enzyme in polyamine biosynthesis is ODC (ornithine decarboxylase) that converts ornithine to putrescine. Spermidine is then synthesized from putrescine by spermidine synthase, and spermine from spermidine. Over the course of the study, spermidine, diacetylspermadine and N1, N12-diacetylspermine increased in the feces receiving Control, Drug or Combo treatments. Conversely, these levels remained low in the Microbe group (Table 23). Since no differences in putrescine were observed, altered spermidine synthase activity could explain these findings. Polyamines stimulate mucosal growth and impacts intestinal enzyme activity (Wang, J. Y., et al., Stimulation of proximal small intestinal mucosal growth by luminal polyamines. Am J Physiol, 1991. 261(3 Pt 1): p. G504-11). Potential bacterial sources of polyamines include species of Bacteroides, Fusobacterium, and Clostridium (Matsumoto, M. and Y. Benno., Microbiol Immunol, 2007. 51(1): p. 25-35).

TABLE 23
Polyamines in mouse fecal samples at time T7. Ratio of the mean
peak areas for the specified metabolites in each group relative to
the control group. Up o rdown arrows indicate whether the
increase or decrease in the treatment relative to the control is
significant based on Welch’s two-sample t-test with p < 0.05.

| Compound | Microbe T7 | | Drug T7 | Combo T7 | |
|---|---|---|---|---|---|
| spermidine | 0.23 | ↓ | 1.46 | 0.75 | |
| diacetyl spermidine | 0.27 | ↓ | 0.91 | 0.79 | ↓ |
| N1,N12-diacetyl spermine | 0.25 | ↓ | 1.18 | 0.87 | |

Nucleotides are the building blocks for DNA and RNA biosynthesis, and they are composed of a nitrogenous base, a five-carbon sugar, and at least one phosphate group. Nucleotides carry energy, participate in cell signaling, and are incorporated into important cofactors. Nucleotides can be synthesized de novo or recycled through salvage pathways. In energy-preserving salvage reactions, nucleosides and free bases generated by DNA and RNA breakdown are converted back to nucleotide monophosphates, allowing them to re-enter the pathways of nucleotide biosynthesis (inter-conversion). Thus, nucleotide levels may reflect epithelial cell turnover. Nucleotides tended to decline in response to the Microbe treatment. 5′-AMP, 5′-GMP and 5′-CMIP were notable exceptions although the biological meaning of these changes remains unknown (Table 24). These nucleic monophosphates may serve as signaling molecules or reflect the degradation of nucleotides.

TABLE 24
Nucleotide synthesis, degradation, and salvage intermediates in
mouse fecal samples at time T7. Ratio of the mean peak
areas for the specified metabolites in each group
relative to the control group. Up or down arrows indicate
whether the increase or decrease in the treatment relative
to the control is significant
based on Welch′s two-sample t-test with p <0.05.

| | Microbe | Drug | Combo |
|---|---|---|---|
| Compound | T7 | T7 | T7 |
| Inosine | 0.51 | 1.59 | 0.88 |
| Hypoxanthine | 0.44 | 1.17 | 0.54 ↓ |
| Xanthine | 0.23 ↓ | 1.29 | 0.61 |
| Xanthosine | 0.65 | 1.31 | 0.40 |
| 2′-deoxyinosine | 0.48 | 1.16 | 0.68 |
| Urate | 0.33 ↓ | 1.15 | 0.91 |
| Allantoin | 1.1 | 1.07 | 0.61 |
| 1-methylhypoxanthine | 0.66 | 0.96 | 0.75 |
| AMP | 3.41 ↑ | 0.99 | 4.50 ↑ |
| 3′-AMP | 0.02 ↓ | 0.11 | 0.20 ↓ |
| Adenosine-2′,3′-cyclic | 0 ↓ | 0.01 ↓ | 0.04 ↓ |
| monophosphate | | | |
| Adenosine | 0.07 ↓ | 0.65 | 0.96 |
| Adenine | 0.23 ↓ | 0.51 ↓ | 0.92 |
| l-methyladenine | 0.27 ↓ | 1.24 | 0.37 ↓ |
| N1-methyladenosine | 1.24 | 1.05 | 0.08 |
| 2′-deoxyadenosine 5′- | 1.62 | 1.09 | 3.61 ↑ |
| monophosphate | | | |
| 2′-deoxyadenosine | 0.28 ↓ | 0.76 ↓ | 0.84 |
| 3′-GMP | 0.15 | 0.98 | 0.56 |
| Guanosine-2′,3′-cyclic | 0.13 ↓ | 0.68 | 0.43 ↓ |
| monophosphate | | | |
| Guanosine | 0.41 | 1.73 | 0.96 |
| Guanine | 0.62 | 0.83 | 0.67 |
| 7-methylguanine | 0.53 ↓ | 1.18 | 0.53 |
| 8-hydroxyguanine | 0.53 | 1.12 | 0.94 |
| dGMP | 1.22 | 1 | 1.9 |
| 2′-deoxyguanosine | 0.64 | 0.92 | 0.88 |
| N-carbamoylaspartate | 0.14 ↓ | 0.89 | 0.25 ↓ |
| orotate | 0.14 ↓ | 0.97 | 0.29 ↓ |
| UMP | 1.25 | 0.84 | 1.66 ↑ |
| 3′-UMP | 0.29 | 0.98 | 0.89 |
| Uridine-2′,3′-cyclic | 0.13 ↓ | 0.59 | 0.45 |
| monophosphate | | | |
| Uridine | 0.82 | 1.12 | 1 |
| Uracil | 0.24 ↓ | 1.26 | 0.55 |
| Pseudouridine | 0.14 ↓ | 1.16 | 0.29 ↓ |
| 5,6-dihydrouridine | 0.24 ↓ | 1.3 | 0.49 |
| 2′-O-methyluridine | 0.11 ↓ | 1.41 | 0.46 |
| 5-methyluridine | 0.29 | 2.28 | 0.50 |
| 2′-deoxyuridine | 0.44 | 1.29 | 0.71 |
| 3-ureidopropionate | 0.09 ↓ | 1.37 | 0.37 |
| Beta-alanine | 0.21 ↓ | 1.16 | 0.28 ↓ |
| 5′-CMP | 2.84 ↑ | 1 | 3.32 ↑ |
| 3′-CMP | 0.44 ↓ | 1.14 | 0.83 |
| Cytidine 2′,3′-cyclic | 0.07 ↓ | 0.4 | 0.29 ↓ |
| monophosphate | | | |
| Cytidine | 0.81 | 0.87 | 1.15 |
| Cytosine | 0.56 | 0.43 | 1.39 |
| 5-methylcytidine | 0.45 | 0.93 | 0.47 ↓ |
| 5-methylcytosine | 0.72 | 1.15 | 0.99 |
| 2′-deoxycytidine 5′- | 1.74 | 0.87 | 2.51 ↑ |
| monophosphate | | | |
| 2′-deoxycytidine | 1.2 | 0.86 | 1.47 |
| 2′-O-methylcytidine | 0.66 | 1.17 | 0.92 |
| 5-methyl-2′-deoxycytidine | 1.06 | 0.89 | 1.11 |
| Thymidine 5′-monophosphate | 1.69 | 0.81 | 2.26 ↑ |
| Thymidine | 0.66 | 1.21 | 0.8 |
| thymine | 0.17 ↓ | 1.56 | 0.54 |
| 3-aminoisobutyrate | 0.8 | 1.31 | 0.86 |

Most dietary triacylglycerol (TAG) digestion is completed in the lumen of the small intestine. The products of TAG digestion, primarily 2-monoacylglycerols (MAG), fatty acids (FA), cholesterol, and lysophospholipids combine with bile salts, forming micelles. The lipid contents of micelles then diffuse into the enterocytes in the distal duodenum and the jejunum, whereas the bile salts are absorbed in the ileum. Within the enterocytes, TAG, cholesterol ester, and phospholipids are reformed from MAG, FA, cholesterol, and lysophospholipids. These reformed lipids are then incorporated into the lipoprotein chylomicrons, from which tissues like skeletal muscle, adipose tissue, and liver can release and take up free FA. Phospholipids were consistently elevated only in the Microbe monotherapy group (Table 25). Microbe treatment may have impacted membrane stability and potentially reflect cellular turnover. This would be consistent with changes in nucleotide levels. Interestingly, these elevations were not observed in the Combo treatment groups, suggesting that the Drug treatment may have negated this influence of Microbe exposure. In addition to dietary sources, these phospholipids could be the result of the shedding of intestinal epithelial cells.

TABLE 25
Phospholipids and related species in mouse fecal samples at time
T7. Ratio of the mean peak areas for the specified metabolites in each group
relative to the control group. Up or down arrows indicate whether the
increase or decrease in the treatment relative to the control is significant
based on Welch′s two-sample t-test with p <0.05.

| | Microbe | Drug | Combo |
|---|---|---|---|
| Compound | T7 | T7 | T7 |
| 1,2-dipalmitoyl-GPC | 1.13 | 0.72 | 0.74 ↓ |
| 1-palmitoyl-2-oleoyl-GPC | 1.43 | 0.82 | 0.82 |
| 1-palmitoly-2-linoleoyl-GPC | 1.71 ↑ | 0.84 | 0.77 |
| 1-stearoyl-2-arachidonoyl-GPC | 0.78 | 0.88 | 0.94 |
| 1-oleoyl-2-linoleoyl-GPC | 1.95 ↑ | 0.82 | 0.66 |
| 1,2-dilinoleoyl-GPC | 2.21 ↑ | 0.80 | 0.62 |
| 1-linoleoyl-2-linolenoyl-GPC | 1.98 ↑ | 0.74 | 0.61 |
| 1-palmitoyl-2-linoleoyl-GPE | 1.61 | 1.03 | 1.06 |
| 1-stearoyl-2-arachidonoyl-GPE | 1.27 | 0/.81 | 1.07 |
| 1-oleoyl-2-linoleoyl-GPE | 1.61 | 0.79 | 0.77 |
| 1,2-dilinoleoyl-GPE | 2.11 ↑ | 0.77 | 0.6 |
| 1-palmitoyl-2-oleoyl-GPI | 3.20 ↑ | 0.94 | 1.19 |
| 1-palmitoyl-2-linoleoyl-GPI | 3.03 ↑ | 0.81 | 1.01 |
| 1-oleoyl-GPA | 0.9 | 0.85 | 0.67 |
| 1-linoleoyl-GPA | 1.54 ↑ | 0.83 | 1.3 |
| 1-palmitoyl-GPC | 2.71 ↑ | 0.88 | 1.44 |
| 2-palmitoyl-GPC | 3.89 ↑ | 1.01 | 2.17 ↑ |
| 1-stearoyl-GPC | 1.26 | 0.85 | 1.27 |
| 1-oleoyl-GPC | 2.93 ↑ | 1.01 | 1.66 |
| 1-linoleoyl-GPA | 3.78 ↑ | 0.87 | 1.54 |
| 1-lignoceroyl-GPC | 1.07 | 0.95 | 1 |
| 1-palmitoyl-GPE | 1.92 ↑ | 1.22 | 1.28 |
| 1-stearoyl-GPE | 0.75 | 0.86 | 1.32 |
| 2-stearoyl-GPE | 0.63 | 0.72 | 1.58 |
| 1-oleoyl-GPE | 1.78 ↑ | 1.16 | 1.24 |
| 1-linoleoyl-GPE | 3.22 ↑ | 0.89 | 1.28 |
| 1-palmitoyl-GPS | 3.41 ↑ | 1.83 ↑ | 2.48 ↑ |
| 1-linoleoyl-GPG | 1.11 | 1.18 | 1.27 |
| 1-palmitoyl-GPI | 3.13 ↑ | 0.67 | 1.33 |
| 1-stearoyl-GPI | 1.38 | 0.93 | 1.87 |
| 1-oleoyl-GPI | 2.90 ↑ | 0.84 | 2.92 |
| 1-linoleoyl-GPI | 3.28 ↑ | 0.93 | 2.68 |

Nicotinamide adenine dinucleotide (NAD⁺) is a coenzyme that plays an essential role in energy metabolism and redox status. NAD⁺ can be synthesized from the amino acid tryptophan through intermediates including kynurenine and quinolinate or salvaged from nicotinic acid and nicotinamide. Prokaryotic and eukaryotic NAD synthetic pathways are similar. Metabolites involved in NAD metabolism were lower in the Combo group at T7, and to a lesser extent the Microbe group (Table 26). Declines in NAD⁺ metabolites in the feces may reflect retention within the colon or decreased production. Increasing NAD⁺ levels in aged mice decreases colon degradation and increases motility (Zhu, X., et al., Nicotinamide adenine dinucleotide replenishment rescues colon degeneration in aged mice. Signal Transduct Target Ther, 2017. 2: p. 17017).

TABLE 26
Nicotinamide and related metabolites in mouse
fecal samples at time T7. Ratio of the
mean peak areas for the specified
metabolites in each group relative to the
control group. Up or down arrows indicate whether
the increase or decrease in the treatment
relative to the control is significant based on
Welch′s two-sample t-test with p <0.05.

| | Microbe | Drug | Combo |
|---|---|---|---|
| Compound | T7 | T7 | T7 |
| N1-methyl-4-pyridone-3- | 0.65 | 0.89 | 0.45 |
| carboxamide | | | |
| N′-methylnicotinate | 0.51 | 1.04 | 0.53 ↓ |
| 1-methylnicotinamide | 1.01 | 1.03 | 0.87 |
| Nicotinamide | 1.33 | 0.94 | 1 |
| Nicotinate ribonucleoside | 0.71 | 0.47 | 0.44 ↓ |
| Nicotinate | 0.37 ↓ | 1.25 | 0.52 ↓ |
| quinolinate | 0.68 | 1.05 | 0.65 ↓ |

Metabolomics data was used to determine metabolic signatures that could differentiate response to checkpoint inhibitor treatment. Of the mice receiving anti-CTLA4, responders to the treatment (R) were defined as those mice with tumor size less than 400 mm³ at the end of the study (21 days from first treatment). Those with tumor size greater than 400 mm³ were considered non-responders (NR). Of the 16 mice given anti-CTLA4 in the metabolomics study (Microbe and Combo groups), there were 12 responders and 4 non-responders.

High level views of the responder data demonstrate relatively low numbers of metabolites were significantly different between R and NR during the study (9% at T1, 6% at T4 and 4% at T7). However, there were clear differences in specific metabolites, though each only at a specific timepoint. Guanosine 3′-monophosphate (3′-GMP) and guanosine-2′,3′-cyclic monophosphate was present in R but not detected in any NR at T1. At T4, multiple primary and secondary bile acids were elevated in the feces of R compared to NR (Table 27). Bile acids are necessary for the efficient absorption of dietary lipids. They are synthesized and conjugated in the liver and secreted into the intestine via the bile duct. Most of the bile acid pool is reabsorbed into enterohepatic circulation; however, a small percentage is excreted in the feces. Interestingly, the differences observed here seemed to be unique to taurine-conjugated bile acids. Taurine levels were not different between these groups at any timepoint; however, cysteine, a precursor to taurine was lower in R versus NR at T1. Secondary bile acids are generated by the gut microbiota, and thus differences in these metabolites may reflect differences in microbial population or metabolism. At T7, diacylglycerols (DAGs) and monoacylglycerols (MAGs) were lower in R versus NR at T7 (Table 28). The bulk of DAGs and MAGs in the colon are derived from dietary sources. Assuming the dietary intake was identical between mice included in the study, changes in these metabolites likely reflect differences in digestion and absorption of these metabolites between R and NR.

TABLE 27
Primary and secondary bile acids in mouse fecal samples at each
timepoint. Ratio of the mean peak areas for the specified metabolites
in responders (R) relative to non-responders (NR). Up or down arrows
indicate the increase or decrease in the treatment relative to the control is
significant based on Welch′s two-sample t-test with p <0.05.

| | R/NR | R/NR | R/NR |
|---|---|---|---|
| Compound | T1 | T4 | T7 |
| Taurocholate | 0.9 | 4.81 ↑ | 0.85 |
| Tauro-beta-muricholate | 0.56 | 5.34 ↑ | 1.5 |
| Taurodeoxycholate | 0.49 | 15.26 ↑ | 1.31 |
| Taurolithocholate | 0.87 | 5.95 ↑ | 1.41 |
| Taurohyodeoxycholic acid | 0.54 | 7.20 ↑ | 1.13 |

TABLE 28
Monoacylglycerols and diacylglycerols in
mouse fecal samples at each timepoint. Ratio of
the mean peak areas for the specified metabolites in
responders (R) relative to non-responders (NR).
Up or down arrows indicate the
increase or decrease in the treatment relative
to the control is significant based on
Welch′s two-sample t-test with p <0.05.

| | R/NR | R/NR | R/NR |
|---|---|---|---|
| Compound | T1 | T4 | T7 |
| 1-myristoylglycerol | 0.91 | 0.71 | 0.73 |
| 1-palmitoylglycerol | 0.96 | 1.11 | 0.61 ↓ |
| 1-oleoylglycerol | 0.88 | 1.46 | 0.47 ↓ |
| 1-linoleoylglycerol | 0.94 | 2.03 | 0.41 ↓ |
| 1-linolenoylglycerol | 1.04 | 2.24 | 0.44 ↓ |
| 2-palmitoylglycerol | 0.91 | 1.05 | 0.63 ↓ |
| 2-oleoylglycerol | 0.99 | 1.2 | 0.47 ↓ |
| 2-linoleoylglycerol | 0.98 | 1.47 | 0.41 ↓ |
| 1-heptadecenoylglycerol | 0.93 | 1.69 | 0.52 ↓ |
| Palmitoyl-linoleoyl-glycerol | 0.89 | 1.36 | 0.63 ↓ |
| Oleoyl-oleoyl-glycerol | 0.82 | 1.09 | 0.63 ↓ |
| Oleoyl-linoleoyl-glycerol | 0.84 | 1.33 | 0.66 ↓ |
| Linoleoyl-lineoyl-glycerol | 0.84 | 1.54 | 0.63 ↓ |
| Linoleoyl-linolenoyl-glycerol | 0.91 | 1.61 | 0.60 ↓ |

### Metabolomics Performed on Fecal Samples

In a separate experiment, metabolomics was performed on fecal samples taken from mice treated with anti-CTLA-4 only and the group treated with anti-CTLA-4 in combination with microbial mix 2. In the tables and figures that follow, these are referred to as the Drug (D) and Drug+Microbe (D+M) groups. Samples were processed from timepoint 2 (T2), 48 hours after the first treatment dose; timepoint 4 (T4), 10 days from start and 48 hours after the 3^{rd} treatment dose; and timepoint 6 (T6), 17 days from start and 48 hours after the 5^{th} treatment dose. All mice in the study were classified as responders or non-responders to CTLA-4 treatment. responders to the treatment (R) were defined as those mice with tumor size less than 400 mm³ at the end of the study (21 days from first treatment). Those with tumor size greater than 400 mm³ were considered non-responders (NR). Of the 16 mice given anti-CTLA4 in the study, there were 8 responders and 8 non-responders.

As in the above example, instrument variability was determined by calculation of the median relative s.d. (RSD) for the standards that were added to each sample before injection into the mass spectrometers (median RSDs were determined to be 3%). Overall process variability was determined by calculating the median RSD for all endogenous metabolites (i.e., noninstrument standards) present in 90% or more of the pooled technical-replicate samples (median RSD=10%, n=802 metabolites).

Several metabolites were differentially present in the R and NR groups, as summarized in Table 29. Proline is consistently elevated in NR samples but only significantly at the mid-time-point. Correlation analysis shows that, although proline is the sentinel signal, the top correlating metabolites to its abundance across the samples are primarily other amino acids. Hence, amino acids generally increase in NR samples at the mid-point. The increase observed in the NR samples in the feces reflects a difference in the potential availability for the tumor for anabolic processes such as protein synthesis. Also elevated in responder samples were particular sugars, mannose and myo-inositol, and trace amines. Mannose (an epimer of glucose) and myo-inositol are both monosaccharides that can be made from glucose and they are abundant in the diet. Mannose is most prominently known for its role in posttranslational modification of proteins through N-linked glycosylation while inositol is most known for its role as a second messenger in the form of inositol phosphates. However, the increase in abundance in the feces of NR animals most plausibly indicates differences in either the use or potential use of these sugars as carbon sources by microbes within the lumen of the intestine. Trace amines such as tyramine, tryptamine and phenethylamine are best known for having neuroactive activity. They are present in the diet and can be produced by the microbiota. All three were detected in this study but only phenethylamine was identified as significant for differences between R and NR groups. These amines act through trace amine-associated receptors (TAARs). TAAR1 may regulate immune responses through leukocyte differentiation and activation. So, the elevation in phenylethylamine in NR samples could reflect the potential to modulate the immune response.

Steroids were more abundant in the responder group, particularly at the last timepoint. Steroids include progestogens, androgens, estrogens, glucocorticoids, and mineralocorticoids, and they have vital roles in coordinating changes in metabolism, inflammation, and immune function. Since the steroids detected in this data all change in a similar manner and are from 3 of these 5 classes of steroids, a general change in steroid metabolism—perhaps at the earliest steps (cholesterol conversion to pregnenolone) is most likely.

TABLE 29
Select metabolites with different abundance in
responders and non-responders to the anti-CTLA-4
treatment. Ratio of the mean peak areas for the
specified metabolites in responders (R) relative
to non-responders (NR) are shown.
Up or down arrows indicate the increase or
decrease in the treatment relative to the
control is significant based on Welch′s
two-sample t-test with p<0.05.

| | R/NR | R/NR | R/NR |
|---|---|---|---|
| Compound | T2 | T4 | T6 |
| Phenethylamine | 0.79 | 0.49 ↓ | 0.77 |
| Proline | 1.08 | 0.58 ↓ | 0.66 |
| Mannose | 1.02 | 0.82 | 0.47 ↓ |
| Myo-inositol | 1.1 | 0.62 ↓ | 0.64 |
| 5alpha-pregnan-3beta,20alpha-diol | 0.94 | 0.77 | 2.88 ↓ |
| disulfate | | | |
| 5alpha-pregnan-diol disulfate | 0.72 | 0.88 | 2.67 ↓ |
| pregnanolone/allopregnanolone | 1.28 | 1.37 | 3.93 ↓ |
| sulfate | | | |
| 5 alpha-androstan-3beta, 17beta-diol | 0.85 | 0.87 | 2.27 ↓ |
| disulfate | | | |

Several metabolites were differentially abundant in the R and NR groups, but only when comparing just those mice treated with D+M. These are listed in Table 30 and include several fatty acids and ceramides as well as serotonin. Serotonin is a key neurotransmitter in the brain-gut axis and significant amounts of peripheral serotonin is synthesized from tryptophan in the gastrointestinal tract by enterochromaffin cells. Various studies have shown that the production of serotonin in the gut is highly influenced by the presence of microbes and their metabolic products. Serotonin trends higher for the non-responder group. The metabolite that serotonin is derived from -tryptophan—does not correlate with the pattern of serotonin change, indicating that the serotonin change is not simply due to changes in tryptophan levels. Tryptophan can also be metabolized into the anti-inflammatory metabolite kynurenine which naturally then has an immunosuppressive role. However, the steady state pools in these fecal samples for kynurenine are unchanged between the R/NR groups.

Certain bile acids also changed between microbe R and NR groups; in particular, minor secondary bile acids that are the products of bacterial metabolism of primary bile acids. Bile acids such as lithocholate (LCA) are reduced with responders and slightly elevated with non-responders. Thus, since these bile acids are by-products of microbial activity, their changes represent the clearest indication of differential microbe activity between the R and NR groups. How this precisely impacts response is not clear but LCA is known to be biologically potent. For example, it is the most powerful known endogenous agonist for a GPCR that regulates vast aspects of metabolism—TGR5. And, bile acids such as LCA also act on receptors involved in the innate immune response G protein-coupled bile acid receptor 1 (GPBAR1 or Takeda G-protein receptor 5) and the Farnesoid-X-Receptor (FXR). GPBAR1 and FXR are reported to modulate the liver and intestinal innate immune system and therefore contribute to tolerance.

TABLE 30
Select metabolites with different abundance in responders
and non-responders to anti-CTLA-4 and microbial
mix 2 combination treatment. Ratio of the
mean peak areas for the specified metabolites in
responders (R) relative to non-responders (NR) are shown,
just for the D + M group. Up or down arrows indicate the
increase or decrease in the treatment relative to the
control is significant based on
Welch′s two-sample t-test with p <0.05.

| | R/NR | R/NR | R/NR |
|---|---|---|---|
| Compound | T2 | T4 | T6 |
| Serotonin | 0.88 | 0.8 | 0.6 ↓ |
| Stearate (18:0) | 1.09 | 1.41 ↑ | 1.03 |
| Arachidate (20:0) | 1.05 | 1.44 ↑ | 1.04 |
| Behenate (22:0) | 0.93 | 1.53 ↑ | 1.1 |
| Nervonate (24:1n9) | 1.01 | 1.75 ↑ | 1.18 |
| 1-palmitoyl-2-arachidonoyl-GPC | 0.68 | 2.73 ↑ | 1.22 |
| (16:0/20:4n6) | | | |
| 1-stearoyl-GPS (18:0) | 0.73 | 2.77 ↑ | 1.09 |
| 1-stearoyl-GPG (18:0) | 2.56 ↑ | 2.23 ↑ | 1.46 |
| 1-stearoyl-GPI (16:0) | 0.66 | 1.8 ↑ | 1.11 |
| 1-palmitoyl-galactosylglycerol (16:0) | 1.33 | 3.18 ↑ | 1.04 |
| Sphingadienine | 1.25 | 0.65 | 0.65 |
| Ceramide (d18:1/14:0, d16:1/16:0) | 1.13 | 1.25 | 0.84 |
| Glycosyl-N-palmitoyl-sphingosine | 0.64 | 0.51 | 0.4 |
| (d18:1/16:0) | | | |
| Eicosanoylsphingosine (d20:1) | 1.25 | 0.94 | 0.7 |
| Pregnenediol disulfate | 1.11 | 1.39 | 1.15 |
| 5alpha-pregnan-3beta, 20alpha-diol | 1.52 | 2.03 ↑ | 2.69 ↑ |
| disulfate | | | |
| 5alpha-pregnan-diol disulfate | 1.01 | 1.71 | 2.64 ↑ |
| Pregnanolone/allopregnanolone | 2.54 | 4.19 | 3.3 ↑ |
| sulfate | | | |
| 5alpha-androstan-3beta, 17beta-diol | 1.41 | 1.22 | 2.03 ↑ |
| disulfate | | | |
| 6-oxolithocholate | 0.97 | 0.41 | 0.51 |
| Isohyodeoxycholate | 1.39 | 0.61 | 0.48 |
| nicotinamide | 1.28 | 2.38 ↑ | 1.81 |

The strongest signal in the data is from microbe treatment (G8 D+M) independent of R/NR. Despite not correlating with response, the changes induced solely by the microbe could provide insights into how the microbe treatment works. Compounds with increased concentration as a result of microbe treatment include those derived from aromatic catabolism, histamine side products, acylglycines, creatine, and NAD+ catabolites. Table 31 indicates the ratio of these metabolites in the D+M treatment group relative to the D group.

Many metabolites that typically arise from microbial catabolism of aromatic amino acids (for example, p-cresol sulfate, p-cresol glucuronide, and 4-hydroxyphenylacetate) and benzoate metabolites (for example, benzoate, hippurate, catechol sulfate, etc.) are increased by microbe treatment. Benzoate metabolites are simple carboxylic acids produced from the microbial degradation of dietary aromatic compounds in the intestine, such as polyphenols, purines and aromatic organic acids. There is precedent for several aromatic amino acid metabolites having biological activity. For example, tryptophan metabolites such as kynurenate, indole, indoxyl sulphate, and indolepropionate, are ligands for the aryl hydrocarbon receptor (AhR). The AhR mediates tumor-promoting effects of dioxin and AhR signaling is also important for the immune response at barrier sites. These examples illustrate the potential for these types of metabolites to have important biological functions, particularly given that many are at fairly high levels in the blood.

While histamine itself is not elevated, many side-products and metabolites of it such as 1-methylhistamine and 1-ribosyl-imidazoleacetate are. This may be important since histamine is involved in inflammatory responses and gut physiology. Histamine may also have specific microbe-induced influences in specific tumors. For example, it was shown that administration of histidine decarboxylase (HDC) from Lactobacillus reuteri resulted in luminal histamine production of Hdc−/− mice and an associated decrease in the number and size of colon tumors. If the microbe treatment has the potential to alter histamine, it may have similar effects as those described in colon tumors.

Several acylglycines are recognized in biology to have important biological properties. Consequently, they are sometimes described as having “endocannabinoid-like” properties. N-arachidonoyl glycine (NAGly) is probably the best studied acylglycine and has been described to influence things such as inflammation, analgesia and, vasorelaxation. In these data, two acylglycines (3,4-methylene heptanoylglycine and picolinoylglycine) increased in the microbe treated group. However, these acylglycines are probably distantly related to versions like NAGly and there are many missing values, likely contributing to the large fold changes. 3,4-methylene heptanoylglycine is glycine conjugated to a short (C7) unsaturated acyl chain, in contrast to long fatty acyl chains that comprise most canonical acylglycines such as the C20-bearing NAGly. Picolinoylglycine is a pyridine-like ring structure conjugated to glycine. Hence, these molecules are highly unique; given the biosynthetic capacity of the microbiome, these unconventional acylglycines may be synthesized by microbes for some biological function. For example, a recent study revealed that one commensal bacteria effector gene family (Cbeg12) encoded enzymes for the production of the acylglycine N-acyl-3-hydroxypalmitoyl-glycine (commendamide).

Creatine is a key metabolite for cellular energy homeostasis in highly dynamic tissues such as brain, skeletal muscle and the gut. Creatine facilitates channeling of high energy phosphates (via phosphocreatine) to maintain ATP generation. In addition to creatine, several of its metabolites are also elevated by microbe treatment. Relevant to the effects in the gut, creatine supplementation is reported to maintain intestinal homeostasis and protect against colitis through rapidly replenishing ATP within colonic epithelial. Notably, gut microbiota produces specific enzymes that can mediate creatine and creatinine breakdown.

Catabolites of NAD+ and/or nicotinamide (NAM) are increased with microbe treatment. NAD+ has numerous critical cellular functions—a coenzyme for energy metabolism and redox status, holistic regulation of metabolism as a substrate for sirtuins, and in DNA repair through Poly (ADP-ribose) polymerases (PARPs). In this study, the methylated metabolites of NAM increased: N1-methyl-2-pyridone-5-carboxamide (2py) and N1-methyl-4-pyridone-3-carboxamide (4py) are increased by microbe treatment, suggesting an upregulation of NAD+/NAM catabolism. 2py and 4py are produced through methylation of NAM by Nicotinamide N-methyltransferase (NNMT) followed by aldehyde oxidase (Aox) oxidation. These reactions have generally been regarded as clearance pathways as 2py and 4py are excreted in the urine. However, recent studies suggest that the products of this pathway may possess biological activity. For example, pharmacological doses of N1-methylnicotinamide (MNAM) is reported to inhibit cyclooxygenase 2 (COX2) and endothelial nitric oxide synthase (eNOS). This may have relevance in an immunotherapy context as inhibition may help combat COX-2 immune evasion.

TABLE 31
Select metabolites with different abundance in mice
treated with microbial mix 2. Ratio of the mean peak
areas for the specified metabolites in the D + M group
compared to the D group is shown. Up or down
arrows indicate the increase or decrease in the
treatment relative to the control is significant based
on Welch′s two-sample t-test with p <0.05.

| | D + M/D | D + M/D | D + M/D |
|---|---|---|---|
| Compound | T2 | T4 | T6 |
| Phenol sulfate | 1 | 55.66 ↑ | 22.17 |
| N-formylphenylalanine | 1.25 | 0.64 ↓ | 0.61 ↓ |
| 4-hydroxyphenylacetate sulfate | 0.96 | 46.99 ↑ | 8.24 ↑ |
| Kynurenate | 0.57 | 3.03 | 1.86 |
| N-formylanthranilic acid | 1.1 | 5.59 ↑ | 1.74 |
| Xanthurenate | 1.04 | 5.43 | 3.04 |
| Serotonin | 1.07 | 0.81 | 0.92 |
| 5-hydroxyindoleacetate | 0.74 | 1.65 | 2.02 |
| Tryptamine | 1.78 ↑ | 1.21 | 1.06 |
| Indole-3-carboxylate | 0.93 | 0.51 ↓ | 0.7 |
| Indoleacetylglycine | 1 | 71.31 ↑ | 5.71 ↑ |
| 3-indoxyl sulfate | 1 | 44.11 | 47.76 |
| Hippurate | 1.31 | 96.34 ↑ | 11.88 |
| Benzoate | 1.47 | 2.76 | 2.32 ↑ |
| 4-hydroxybenzoate | 0.93 | 1.83 ↑ | 0.97 |
| Catechol sulfate | 1 | 4.01 | 4.29 ↑ |
| Imidazole lactate | 0.7 | 2.42 | 1.41 |
| Histamine | 1.25 | 1.09 | 0.82 |
| 1-methylhistamine | 0.61 | 8.99 ↑ | 3.94 |
| 1-methyl-4-imidazoleacetate | 1.06 | 8.52 | 3.13 |
| 1-methyl-5-imidazoleacetate | 1.07 | 0.6 | 0.92 |
| 1-ribosyl-imidazoleacetate | 1 | 27.27 ↑ | 5.14 |
| 3,4-methylene heptanoylglycine | 1 | 16.15 ↑ | 4.83 |
| picolinoylglycine | 1 | 24.6 ↑ | 5.78 |
| Guanidinoacetate | 0.5 | 37.6 ↑ | 9.04 |
| Creatine | 0.47 | 16.4 ↑ | 2.92 |
| Creatinine | 0.41 | 15.91 ↑ | 4.88 |
| 4-guanidinobutanoate | 1.66 | 4.95 ↑ | 3.71 |
| Nicotinate | 1.93 | 0.91 | 0.69 |
| Nicotinate ribonucleoside | 1.28 | 1.37 | 0.5 |
| Nicotinic acid mononucleotide | 1.39 | 1.22 | 0.59 |
| Nicotinamide | 1.43 | 0.78 | 1.16 |
| Nicotinamide ribonucleotide | 0.76 | 0.82 | 0.89 |
| Nicotinamide riboside | 2.06 | 1.2 | 1.03 |
| 1-methylnicotinamide | 1.05 | 2.48 | 12.05 |
| Trigonelline (N′-methylnicotinate) | 0.8 | 10.26 ↑ | 2.28 |
| N1-methyl-2-pyridone-5- | 0.64 | 4.65 ↑ | 3.11 |
| carboxamide | | | |
| N1-methyl-4-pyridone-3- | 0.8 | 9.25 ↑ | 3.67 |
| carboxamide | | | |

### Example 16—Efficacy of Anticancer Live Biotherapeutics as Monotherapies

The results described here were obtained from studies conducted with tumor mouse models evaluating the anticancer efficacy of generated live biotherapeutics as a monotherapy. Microbes, gene functions, and metabolites elucidated as critical for anticancer treatment are relevant for the treatment of viral infections because in both cases a healthy immune response is required to combat the disease. Therefore, it is reasonable to expect that microbes beneficial for immuno-oncology treatment will also be beneficial or even essential for rapid viral clearance.

### Animals and Tumor Model

BALB/c mice were obtained from Shanghai Lingchang Biotechnology Co., Ltd (Shanghai, China). 6-8-week-old female mice are used. For tumor growth experiments, mice are injected subcutaneously with 2.5×10⁵ CT-26 colon cancer tumor cells (Griswold and Corbett (1975) Cancer 36:2441-2444). Tumor size was measured twice a week until endpoint, and tumor volume determined as length×width×0.5.

### Tumor Cell Preparation

Cryo vials containing CT-26 tumor cells are thawed and cultured according to manufacturer's protocol (ATCC CRL-2638). On the day of injection cells are washed in serum free media, counted, and resuspended in cold serum free media at a concentration of 250,000 viable cells/100 μl.

### Flow Cytometry

A whole-blood flow cytometry-based assay is utilized to assess T cell activation in response to microbial treatment. Whole blood via cardiac puncture is collected into an EDTA tube at the end of the experiment. 100 μL of whole mouse blood is transferred to a 15 mL conical tube. 1 mL of RBC Lysis Buffer is added to the tube and allowed to incubate at room temperature for 10 minutes. Lysis is quenched by adding 10 mL of cold DPBS. Samples are centrifuged at 1500 rpm for 5 minutes at 4° C. The pellet is aspirated and resuspend in another 10 mL of cold DPBS. Samples are recentrifuged at 1500 rpm for 5 minutes at 4° C. Samples are resuspended in 500 μL of FACS buffer and transferred to a 96-well plate. Samples are stained with Fixable Viability ef780™ (eBioscience), CD45-PEcy7 (BioLegend), CD3-BV605™ (BioLegend), CD8-AF700™ (BioLegend), and CD4-AF488™ (BioLegend). Stained samples are run on a BD LSRFortessa™ flow cytometer and analyses are performed with FlowJo™ (Tree Star).

### Tumor Challenge and Treatment

Tumor size is routinely monitored by means of a caliper. Stool is collected on day 0 and 48 hours after each subsequent administration of treatment until the end of the study.

To test whether manipulation of the microbial community is effective as a monotherapy, microbial mix 4 was evaluated in the presence or absence of ellagic acid and/or ellagitannin is administered. In some groups, ellagic acid is administered separately via oral gavage (0.2 mL of a 5.5 mg/mL suspension) prior to administration of the microbe cocktails. Each mouse treated by monotherapy is given 200 l of the suspension by oral gavage three times a week for the duration of the study starting from day 1. Tumor growth and tumor-specific T cell responses are compared among the different treatment groups.

### GI Tract Removal and Analysis

After mice are euthanized at the termination of the study, the intact digestive tract of each mouse from stomach to rectum are removed and kept in a 5 ml Eppendorf tube on ice prior to dissection. Forceps are sterilized by soaking in 100% ethanol and then used to remove the intestine length and stretch it on a work surface covered with cellophane. With the use of ethanol-sterilized dissection scissors, 3 cm lengths of the jejunum nearest to the stomach and the ilium nearest to the cecum/large intestine are excised and then each placed with forceps in a 1.5 ml Eppendorf tube and placed on ice. A 2 cm segment of the cecum/ascending colon is then excised, as are 2 cm segments of the transcending colon and the descending colon, and all are placed in 1.5 ml Eppendorf tubes on ice. Dissection instruments are sterilized by dipping in 100% ethanol between each intestine fragment removal. To each tube containing dissected intestinal segments is added 0.5 ml ice cold PBS buffer. A plastic pestle is used to press and massage the intestinal segment in each tube to expel ruminal matter, which is then removed by pipette and placed in a fresh Eppendorf tube. Tubes containing expelled ruminal matter from each intestinal segment are immediately placed on dry ice and then stored for later analyses at −80° C. Remaining intestinal tissues are then rinsed twice by adding and then removing 0.5 ml ice cold PBS. Rinsed intestinal fragment tissues are then frozen on dry ice and then stored at −80° C. for later analysis.

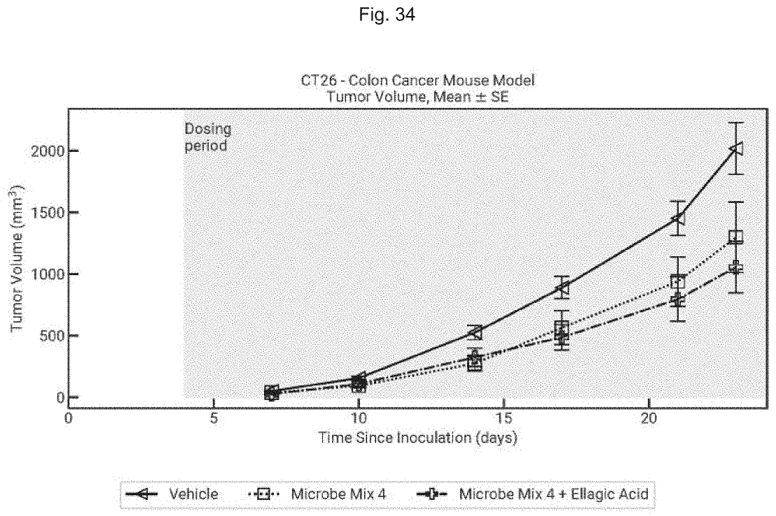

Tumor size is measured in all animals receiving the different microbial treatments. On average, the animals receiving Microbial mix 4 (equal amounts of F. prausnitzii, C. coccoides, R. gnavus, C. scindens, E. lenta, and G. urolithinfaciens) alone or in conjunction with ellagic acid have a reduction in tumor size compared to those receiving vehicle as illustrated in FIG. 34.

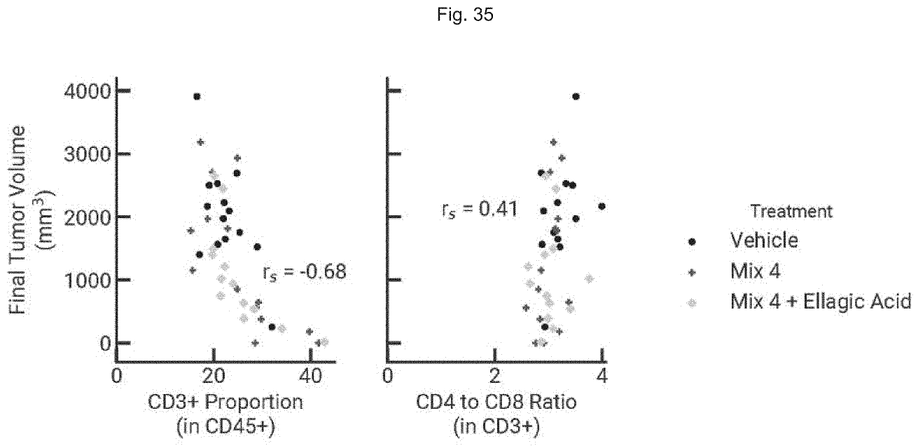

Flow cytometry is used to perform immunophenotyping of mice subjected to cancer receiving the different microbial treatments. Measurements are conducted on both peripheral blood and on the tumor itself, with stains for various cell surface markers. The results show that CD3+ cells, which includes both helper and killer T cells, are upregulated in mice that respond better to therapy. Furthermore, the results also show that mice receiving the therapy had both higher CD3+ proportions as well as much lower final tumor volumes. CD8+T-lymphocytes are also upregulated in the presence of the microbial treatments. These results provide evidence that the microbial mix therapeutic impacts tumor volume via a mechanism of stimulating the CD3+ cells of the immune system as well as cytotoxic CD8+ T cells. Similarly, it has been shown in mice that the commensal microbiota critically regulates the generation of virus-specific CD4 and CD8 T cells and antibody responses following respiratory influenza virus infection (T. Ichinohe et al., Proc. Natl. Acad. Sci. U.S.A 108, 5354-9 (2011)). This further supports that live biotherapeutics described herein that are critical for immuno-oncology treatment will also be beneficial or even essential for rapid viral clearance. Flow cytometry results are graphically presented in FIG. 35.

### Example 17—Therapeutic Effect of Live Biotherapeutics on Efficacy of Cancer Immunotherapy with Antibiotic Pretreatment

The results described here were obtained from studies conducted with tumor mouse models evaluating the anticancer efficacy of generated live biotherapeutics on cancer immunotherapy with antibiotic pretreatment. It has been demonstrated that antibiotic-treated (ABX) mice exhibit impaired innate and adaptive antiviral immune responses and substantially delayed viral clearance after exposure to systemic lymphocytic choriomeningitis virus (LCMV) or mucosal influenza virus (M. C. Abt et al., Immunity. 37, 158-170 (2012)). Microbes, gene functions, and metabolites elucidated as critical for anticancer treatment are relevant for the treatment of viral infections because in both cases a healthy immune response is required to combat the disease. As such, it is reasonable to expect that microbes beneficial for immuno-oncology treatment will also be beneficial or even essential for rapid viral clearance.

Anaerobe Basal Broth Supplemented with Rumen Fluid (ABB+RF)

34.5 grams of anaerobic basal broth dry powder (Fisher Scientific/Oxoid) is combined with 600 ml distilled water and is brought to a gentle boil while stirring on a heated stirplate until the solution clarifies. 150 ml of rumen fluid (Bar Diamond Inc., Parma Id.) that has been centrifuge-clarified is then added, along with 1 ml 2.5 mg/ml resazurin (ACROS Organics™) solution followed by distilled water to one-liter final volume. The medium is kept at 55° C. in a water bath while it is dispensed in 50 ml volumes into 100 ml serum bottles. Nitrogen is bubbled through a metal canula into each bottle for 15 minutes to displace oxygen from the medium, then the bottles are quickly sealed by insertion of a butyl-rubber bung that is secured by a crimped collar. The medium bottles are then sterilized by autoclaving and then stored in the dark until use. L-cysteine is added to 1 mM final concentration to each ABB+RF bottle one hour prior to use to fully reduce the medium prior to inoculation with microorganisms.

### Preparation of Centrifuge-Clarified Rumen Fluid

Rumen fluid is the liquid obtained from the rumen of fistulated cows and is obtained in one-liter volumes from Bar Diamond Inc., Parma Id. The rumen fluid is aliquoted in 50 ml volumes into 50 ml conical tubes and centrifuged at 4000 g for 30 minutes at 4° C. to pellet large fibrous material. After centrifugation the supernatant is decanted into fresh 50 ml conical tubes that are then subjected to centrifugation at 34,000 g for 90 minutes at 4° C. The supernatant from this centrifugation is then decanted into fresh 50 ml conical tubes and stored at −20° C. until use.

### Microorganisms in Mouse Study

The following obligate anaerobic microbes are obtained from the American Type Culture Collection (ATCC): Faecalibacterium prausnitzii (ATCC-27768), Clostridium coccoides (ATCC-29236), Ruminococcus gnavus (ATCC-29149), Clostridium scindens (ATCC-35704), Akkermansia muciniphila (BAA-835), Enterococcus hirae (ATCC-9790), Bacteroides thetaiotamicron (ATCC-29148), Bacteroides caccae (ATCC-43185), Bifidobacterium breve (ATCC-15700), Bifidobacterium longum (ATCC BAA-999) and Gemmiger formicilis (ATCC-27749). Eggerthella lenta (DSM-2243), Gordonibacter urolithinfaciens (DSM-27213), Gordonibacter species CEBAS 4A4; Alistipes indistinctus (DSM-22520), Dorea formicigenerans (DSM-3992), Senegalimassilia anaerobia (DSM-25959), Collinsella aerofaciens (DSM-3979), Adlercreutzia equolifaciens (DSM-19450), Ellagibacter isourolithinifaciens (DSM-104140), Slackia isoflavoniconvertens (DSM-22006), Slackia equolifaciens (DSM-2485) and Paraeggerthella hongkongensis (DSM-16106) are obtained from the Leibnitz Institute-German Collection of Microorganisms and Cell Cultures (DSMZ).

The following organisms were obtained from stool of healthy donors as described in Example 16: Dorea longicatena and Blautia sp. SG-772. Whole genome sequencing of these organisms indicated they are more than 95% identical to the published strains.

### Culture of Individual Microbes for Mouse Study

0.5 ml starter cultures of C. coccoides, R. gnavus, C. scindens, A. muciniphila, E. hirae, B. thetaiotamicron, B. caccae, B. breve, B. longum, G. formicilis, E. lenta, G. urolithinfaciens, A. indistinctus, D. formicigenerans, S. anaerobia, C. aerofaciens, A. equolifaciens, E. isourolithinifaciens, S. isoflavoniconvertens, S. equolifaciens and P. hongkongensis, E. hallii, D. longicatena, and Blautia sp. SG-772 are each inoculated into four 50 ml anaerobic bottles of fully reduced ABB+RF anaerobic medium and cultured at 37° C. F. prausnitzii is inoculated into fifteen 7 ml tubes of YCFAC (Anaerobe Systems) and cultured at 37° C. Cultures are harvested after 48 hours when they achieve 0.1 to 1.0×10⁹ cells/ml as measured by optical absorbance at 600 nm by spectrophotometer (1 OD₆₀₀=1.0×10⁹ cells/ml). Bacterial starter cultures may be modified to achieve 1.0×10¹⁰ cells/ml, 1.0×10¹¹ cells/ml or 1.0×10¹² cell/ml.

To harvest cultures, they are first brought into the anaerobic chamber where they are opened and decanted into 50 ml conical tubes that are tightly capped and sealed by wrapping the caps in parafilm. These are brought out of the anaerobic chamber and then centrifuged at 4000 g for 15 minutes at 4° C. The centrifuged tubes are brought back into the anaerobic chamber where the supernatant is decanted and discarded. The cell pellets are each combined with anoxic Phosphate Buffered Saline with 2.5 mM L-Cysteine and 15% glycerol (PBS-C-G) followed by tight capping and parafilm seal. The capped and sealed tubes are brought out of the anaerobic chamber and are centrifuged at 4000 g for 15 minutes. The culture tubes are again brought into the anaerobic chamber where the supernatant is decanted and discarded. Pelleted cells are resuspended in volumes of PBS-C-G to attain effective cell densities of each microbial strain at 1×10⁹ cells/ml, 1.0×10¹⁰ cells/ml, 1.0×10¹¹ cells/ml or 1.0×10¹² cell/ml.

### Animals and Tumor Model

BALB/c mice are obtained from Jackson laboratory, Taconic farms or Shanghai Lingchang Biotechnology Co., Ltd (Shanghai, China). 6-8-week-old female mice are used. For tumor growth experiments, mice are injected subcutaneously with 2.5×10⁵ CT-26 colon cancer tumor cells (Griswold and Corbett (1975) Cancer 36:2441-2444). Tumor size is measured twice a week until endpoint, and tumor volume determined as length×width×0.5.

### Tumor Cell Preparation

Cryo vials containing CT-26 tumor cells are thawed and cultured according to manufacturer's protocol (ATCC CRL-2638). On the day of injection cells are washed in serum free media, counted, and resuspended in cold serum free media at a concentration of 250,000 viable cells/100 μl. Cells will be prepared for injections by withdrawing 100 μL cell suspension into a 1 ml syringe. The cell suspension and filled syringes will be kept on ice.

### Tumor Implantation

Animals will be prepared for injection using standard approved anesthesia, the mice will be shaved prior to injection. Once mouse at a time will be immobilized and the site of injection will be disinfected with an alcohol swab. 100 μl of the cell suspension will be subcutaneously injected into the rear flank of the mouse. During implantation, a new syringe and needle will be used for every mouse inoculated to minimize tumor ulceration. The cells will be drawn up into a 1 mL syringe (no needle attached) to 150 μL with the 50 μL nearest to the plunger being air and 100 μL of cell suspension. Once the cells are drawn up the needle will be attached (without priming the needle). For implant, lift up or tent the skin using forceps to ensure a subcutaneous injection. Inject the cells, twist the syringe/needle and then pull the needle out. Mice will be marked by ear tagging.

### Antibiotics Protocols

Mice are treated daily with 200 μL of water or antibiotics via oral gavage 1-2 weeks before tumor implantation and continued for a duration of 2-3 weeks. Mouse fecal samples were collected twice a week for 5 collections in total (timepoints 1-5). Animals are given a mix of ampicillin (1 mg/mL)(Alfa Aesar J6380706), gentamicin (1 mg/mL)(Acros Organics AC455310050), metronidazole (1 mg/mL)(Acros Organics AC210440050), neomycin (1 mg/mL)(Alfa Aesar AAJ6149922), and vancomycin (0.5 mg/mL)(Alfa Aesar J6279006) via oral gavage. Antibiotic activity is analyzed by macroscopic changes observed at the level of caecum (dilatation) and by cultivating the fecal pellets resuspended in BHI+15% glycerol on blood agar and anaerobic blood agar plates for 48h at 37° C. with 5% CO2 for aerobic conditions or in anaerobic conditions respectively. 16S RNA and Whole Genome Sequencing are applied to determine the distribution of organisms in fecal samples collected from the water and antibiotic treated groups at both the phylum and genus level, and the distribution is compared across all collected fecal samples. PCA is used to classify all samples of mice without antibiotic treatment, showing that samples with the same microbial treatment type cluster together. Mice are treated with antibiotics or water for two weeks and fecal samples are collected at three different time points.

Isolation of Lamina Propria Cells from Small Intestine

Whole duodenum and ileum are harvested, Peyer's patches are removed, as well as all fat residues and fecal content. Small fragments are obtained by cutting them first longitudinally along the length and then transversally into pieces of 1-2 cm length. After removing the intra-epithelial lymphocytes (IELs), the gut pieces are further cut and incubated with 0.25 mg/ml collagenase VIII and 10 U/ml DNaseI for 40 min at 37° C. under shaking to isolate lamina propria cells (LPCs). After digestion, intestinal pieces are mashed on a cell strainer. For FACS analysis, cell suspensions are subjected to a percoll gradient for 20 min at 2100 RPM, while for RNA extraction, cells are directly lysed in RNALater buffer (Thermo Fisher Scientific) and frozen at −80° C.

### Analyses of Dendritic Cell Subsets in Treated Mice

Cell suspensions from mouse spleen and lymph nodes are prepared by digestion with collagenase and DNase for 60 min and subsequently strained through a 70 mm mesh. Colonic and small intestinal lymphocytes are isolated as previously described (Viaud, S. et al. Science (80-.). 342, 971-976 (2013). In brief, cecum, colon and small intestine are digested in PBS containing 5 mM EDTA and 2 mM DTT shaking at 37° C. A plastic pestle is used to press and massage the intestinal segment in each tube to expel ruminal matter, which is then removed by pipette and placed in a fresh Eppendorf tube. Tubes containing expelled ruminal matter from each intestinal segment are immediately placed on dry ice and then stored for later analyses at −80° C. Remaining intestinal tissues are then rinsed twice by adding and then removing 0.5 ml ice cold PBS. Rinsed intestinal fragment tissues are then frozen on dry ice in RNALater (Thermo Fisher Scientific) and then stored at −80° C. for later analysis.

After initial digestion colonic and small intestinal tissue pieces are digested in collagenase/DNase containing RPMI medium for 30 min. Tissue pieces are further strained through a 70 mm mesh. For flow cytometry analyses, cell suspensions are stained with antibodies against the following surface markers: CD11c (N418), CD11b (M1/70), Ly6c (HK1.4), MHC class II (M5/114.15.2), CD24 (M1/69), CD64 (X54-5/7.1), CD317 (ebio927), CD45 (30-F11), F4/80 (C1:A3-1), CD8a (53-6.7). DAPIis used for dead cell exclusion. Antibodies are purchased from eBiosciences, BD Biosciences or BioLegend respectively. Cell populations are gated as follows: small intestine (migratory fraction): CD103+DC (CD45+CD11c+MHC-II+CD103+CD24+), CD11b+CD103+(CD45+CD11c+MHC-II+CD103+CD11b+CD24+), CD11b+(CD45+CD11c+MHC-II+CD11b+CD24+), inflammatory DC (CD45+CD11c+MHC-II+CD11b+CD64+Ly6c+), large intestine: CD103+DC (CD45+CD11c+MHC-II+CD103+CD24+), CD11b+(CD45+CD11c+MHC-II+CD11b+CD24+), inflammatory DC (CD45+CD11c+MHC-II+CD11b+CD64+Ly6c+).

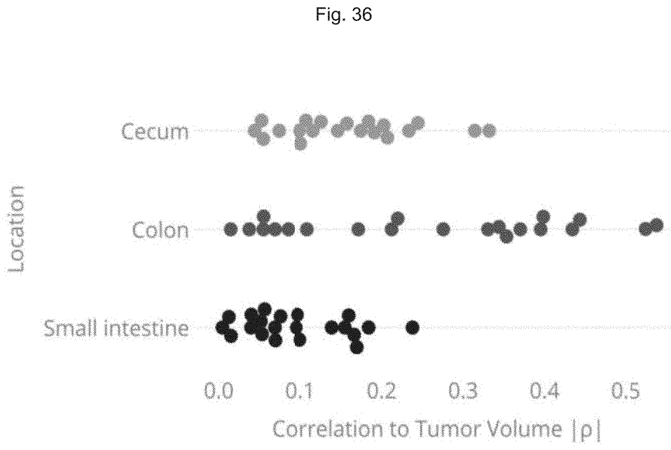

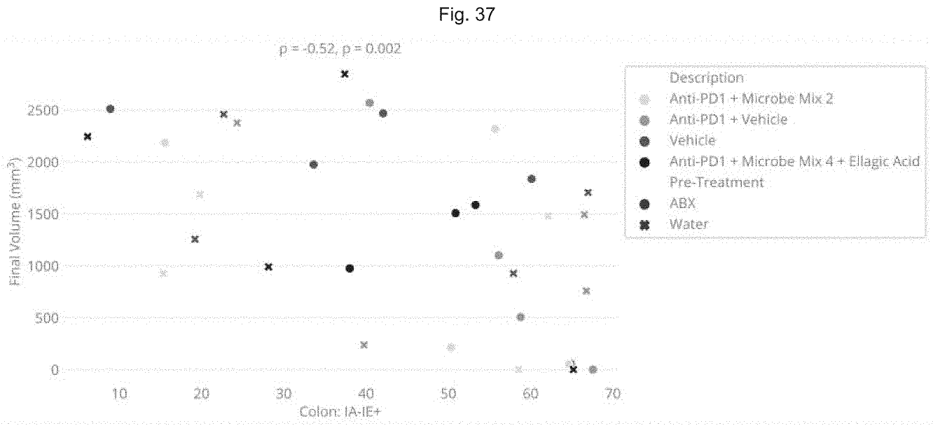

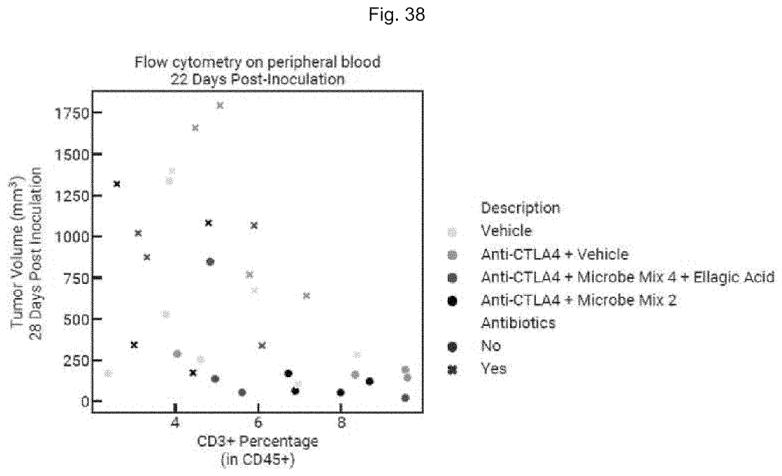

Flow cytometry analyses were performed on small intestine, cecum and colon tissue collected from mice pretreated with water and antibiotics and treatments including vehicle, anti-PD-1 and vehicle, anti-PD-1 in combination with microbial mix 4 and ellagic acid and anti-PD-1 in combination with microbial mix 2. Spearman correlation was computed between final tumor volume and each flow gate for all treatments in each GI location. Correlations passing a false discovery rate threshold of 0.25 are reported in Table 32. Spearman correlations between each flow gate, final tumor volume and their magnitude by GI location is reported in FIG. 36. The strongest correlations between final tumor volume and the flow results occur in the colon. Final tumor volume for all treatment groups was plotted against the IA/IE (MIC Class II) immune population in the colon, which revealed a statistically significant negative correlation as reported in FIG. 37.

TABLE 32

| Category | P | rho | location |
|---|---|---|---|
| Colon: CD11b-IA-IE+ | 0.001495129 | −0.537953832 | Colon |
| Colon: IA-IE+ | 0.002046607 | −0.524752511 | Colon |
| Colon: Monocytes | 0.011114461 | −0.442977661 | Colon |
| Colon: cDC | 0.013117759 | 0.433810077 | Colon |

### Fecal Microbiota Transplantation (FMT)

Fecal Microbiota Transplantation (FMT) of a favorable gut microbiome into antibiotic treated mice is a method for standardizing microbiome composition. FMT is performed in some experiments with fecal material derived from healthy and cancer patients, as well as mouse stools. Colonization is performed by oral gavage with 200 μl of suspension obtained by homogenizing the fecal samples in PBS. Efficient colonization is first checked before tumor inoculation. Mouse fecal samples are collected 1-2 times during this period. So that the efficacy of the FMT can be evaluated. Following FMT, a rest period of 5-7 days occurs prior to checkpoint inhibitor and/or microbe dosing. Blood and fecal pellets are collected at different time points during the experiment.

### Flow Cytometry of Peripheral Blood

A whole-blood flow cytometry-based assay is utilized to assess T cell activation in response to anti-CTLA-4, anti-PD-1 and microbial treatment. Whole blood via cardiac puncture is collected into an EDTA tube at the end of the experiment. 100 μL of whole mouse blood is transferred to a 15 mL conical tube. 1 mL of RBC Lysis Buffer is added to the tube and allowed to incubate at room temperature for 10 minutes. Lysis is quenched by adding 10 mL of cold DPBS. Samples are centrifuged at 1500 rpm for 5 minutes at 4° C. The pellet is aspirated and resuspend in another 10 mL of cold DPBS. Samples are recentrifuged at 1500 rpm for 5 minutes at 4° C. Samples are resuspended in 500 μL of FACS buffer and transferred to a 96-well plate. Samples are stained with Fixable Viability ef780 (eBioscience), CD45-PEcy7 (BioLegend), CD3-BV605 (BioLegend), CD8-AF700 (BioLegend), and CD4-AF488 (BioLegend). Stained samples are run on a BD LSRFortessa™ flow cytometer and analyses are performed with FlowJo™ (Tree Star).

Flow cytometry analysis was performed on mice and CD3+ percentage is displayed against tumor volume at day 28 post-inoculation as shown in FIG. 38. There is a strong inverse relationship between CD3+ percentage and tumor volume where CD3+ cells are increased by treatment with microbial mixes 2 and 4.

### Tumor Challenge and Treatment

After pre-treatment is complete, animals will be randomized when average tumor volume reaches 40-60 mm³ (Study Day 0). Dosing of Microbes, Vehicle, anti-CTLA-4, anti-PD1 and Ellagic Acid will begin the following day (Study Day 1) below and continue for 3 weeks. Animals are given at least 48 hrs of no treatment between antibiotic pre-treatment and regular study treatment to allow for antibiotics to go through system. Mice are divided into immunotherapy treatment and non-treatment groups. The treatment group is injected intraperitoneally once the tumor reached a size of 40 to 60 mm³ (day 0) with 100 μg anti-PD1 mAb (BioXCell), or with 100 μg anti-PD-L1 mAb, or with 100 μg anti-CTLA-4 mAb (BioXCell) in 100 μl PBS twice a week for three weeks starting from day 1. Tumor size is routinely monitored by means of a caliper. Stool is collected on day 0 and 8 hours after each subsequent administration of treatment until the end of the study.

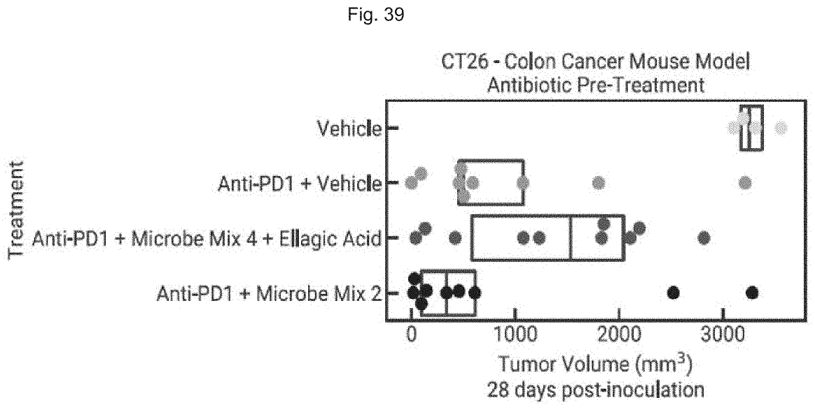

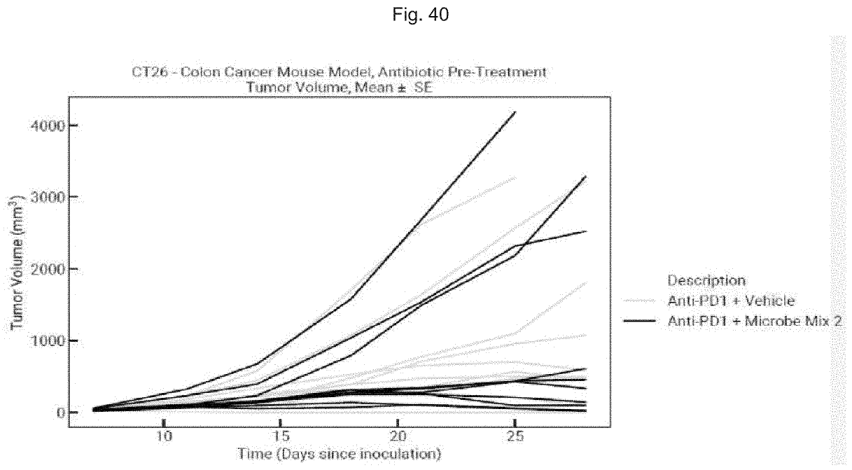

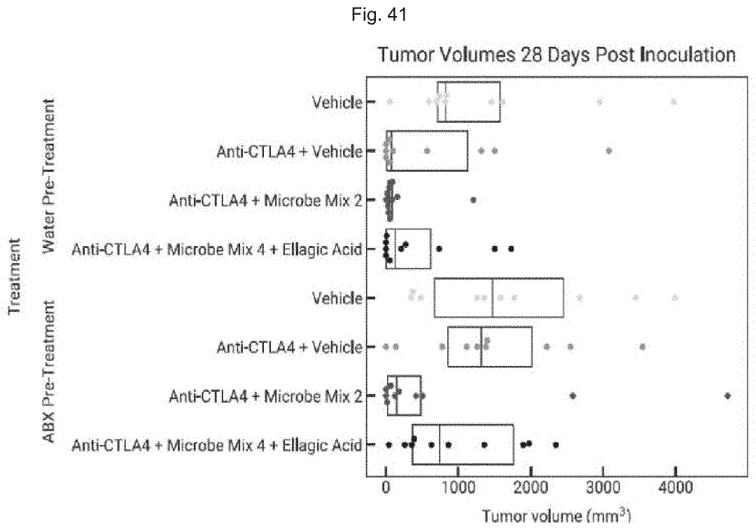

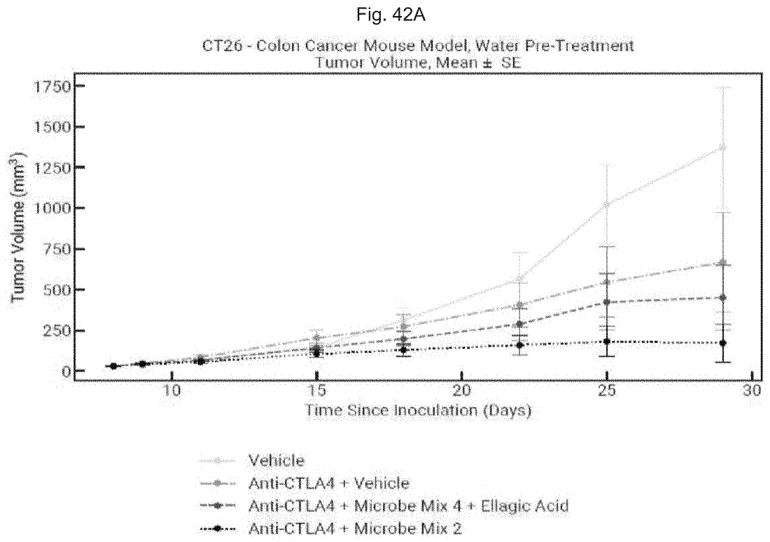

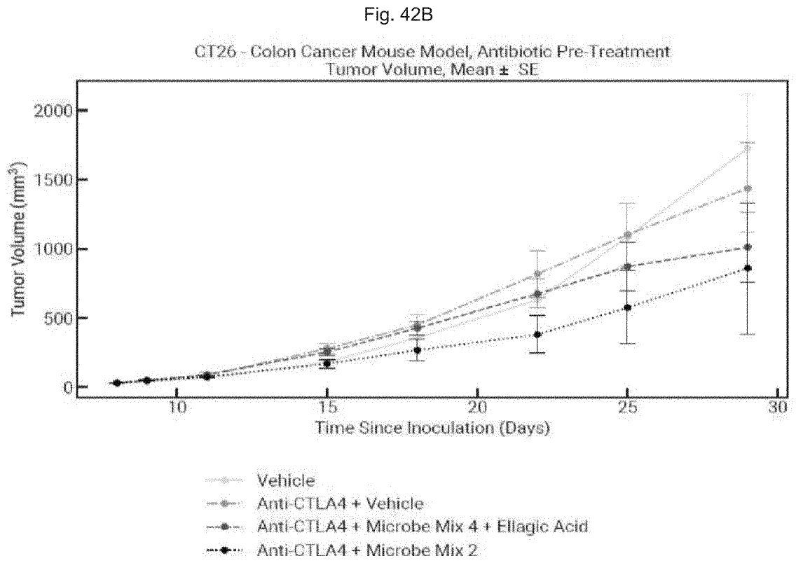

Tumor size was measured in all animals receiving the different microbial treatments, with and without anti-CTLA-4, anti-PD1 or anti-PD-L1 therapy. On average, the animals receiving microbial mix 2 (equal amounts of F. prausnitzii, C. coccoides, R. gnavus, C. scindens, A. muciniphila, and E. hirae) in conjunction with anti-PD1 have a reduction in tumor size compared to those with other microbes or not receiving any anti-PD1 treatment, as illustrated in FIG. 39. Mice treated with microbial mix 2 and the anti-PD1 therapy had reduced tumor growth in contrast to the anti-PD1 monotherapy as shown in FIG. 40. Tumor volumes were measured 28 days post inoculation and displayed by both pre-treatment and treatment groups as shown in FIG. 41. On average, the animals receiving microbial mix 2 (equal amounts of F. prausnitzii, C. coccoides, R. gnavus, C. scindens, A. muciniphila, and E. hirae) in conjunction with anti-CTLA-4 in both pre-treatment groups, have a reduction in tumor size compared to those with other microbes or the anti-CTLA-4 monotherapy. Tumor volumes were measured at multiple time points post-inoculation. Mean and standard error of the mean are displayed for each treatment group within water and antibiotic pre-treatment groups are shown in FIG. 42.

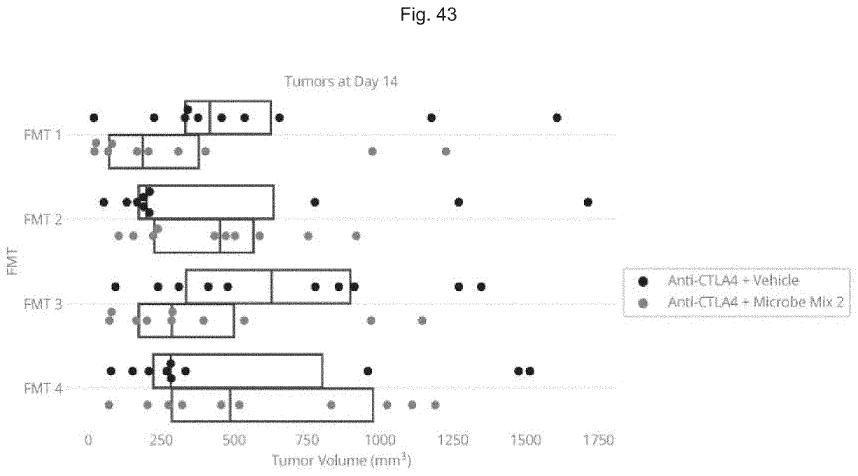

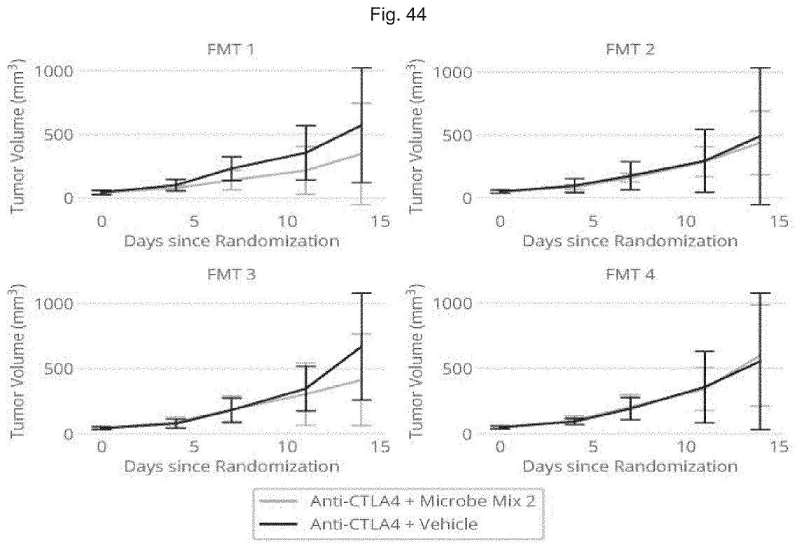

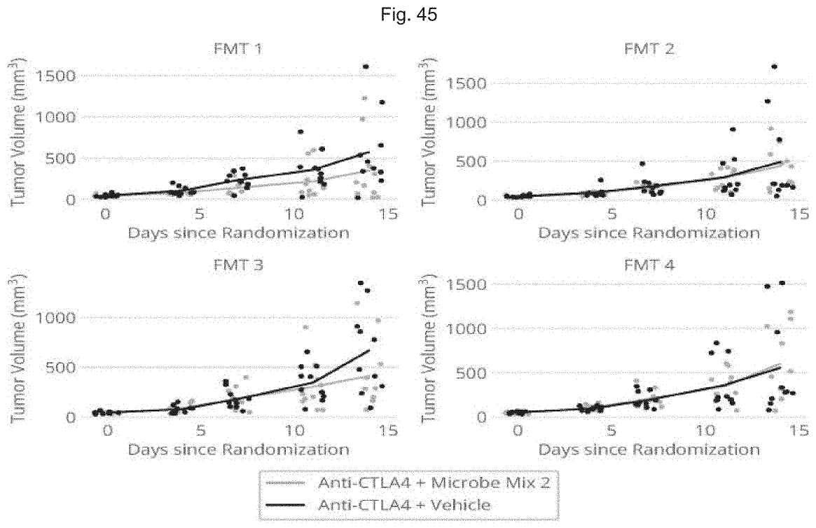

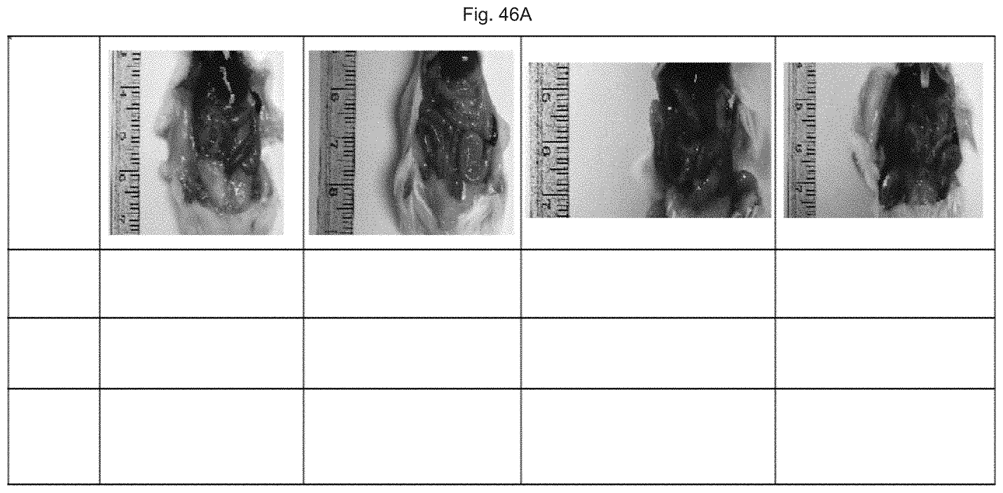

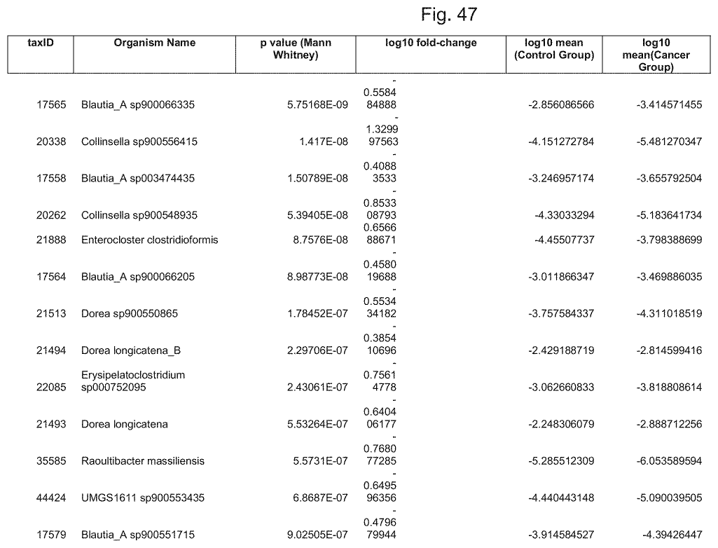

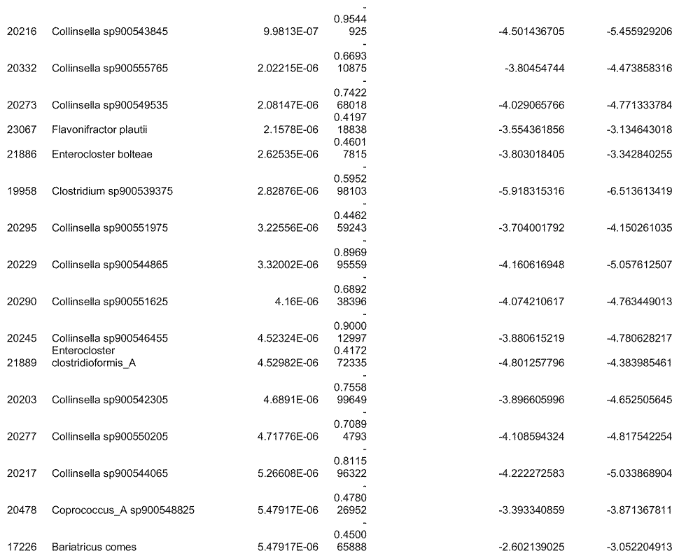

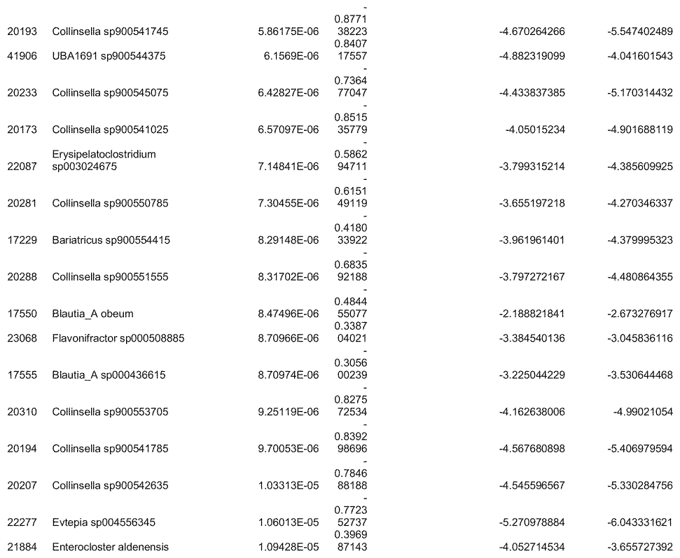

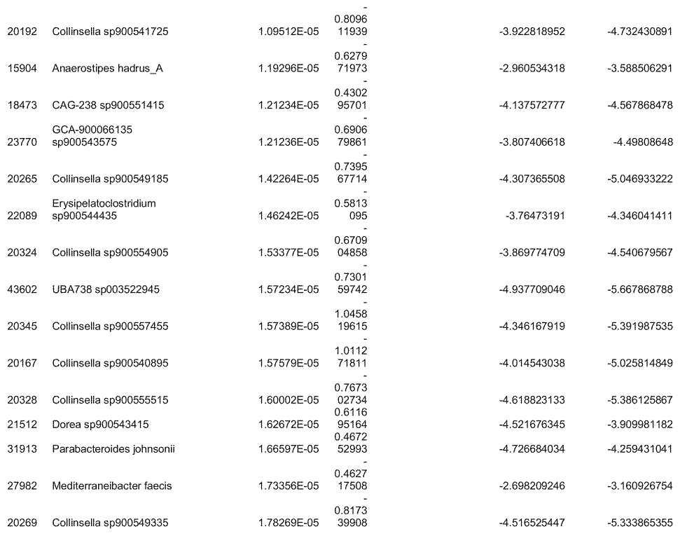

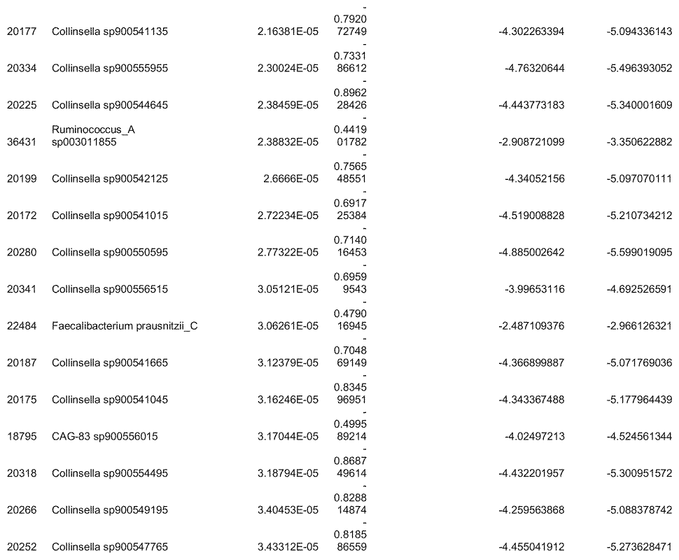

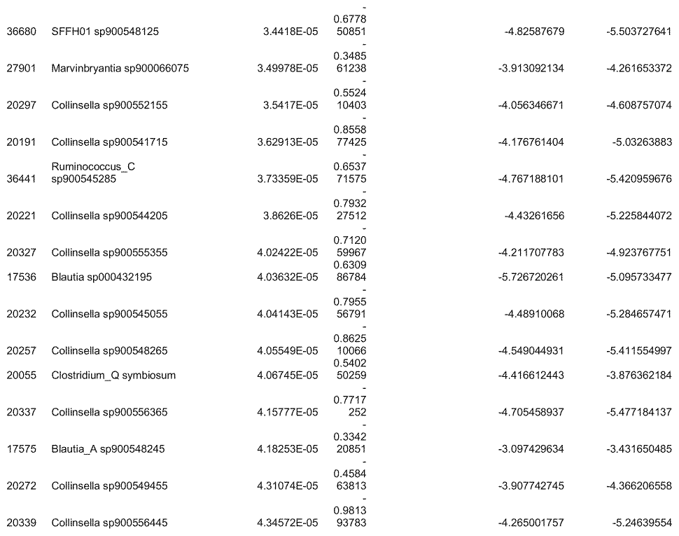

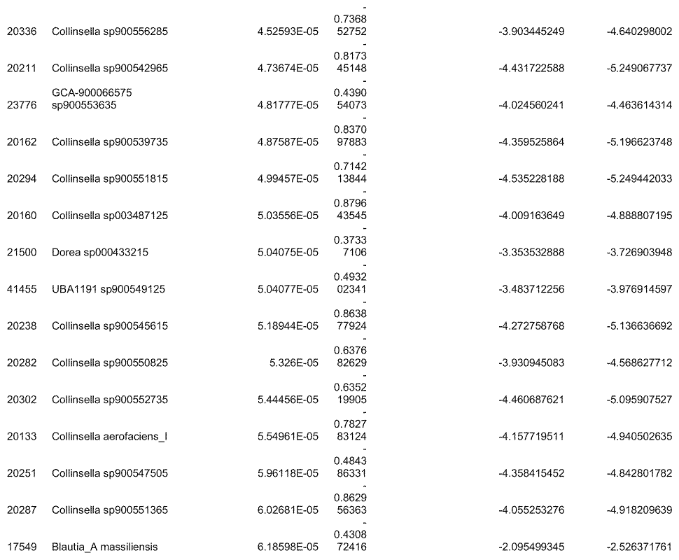

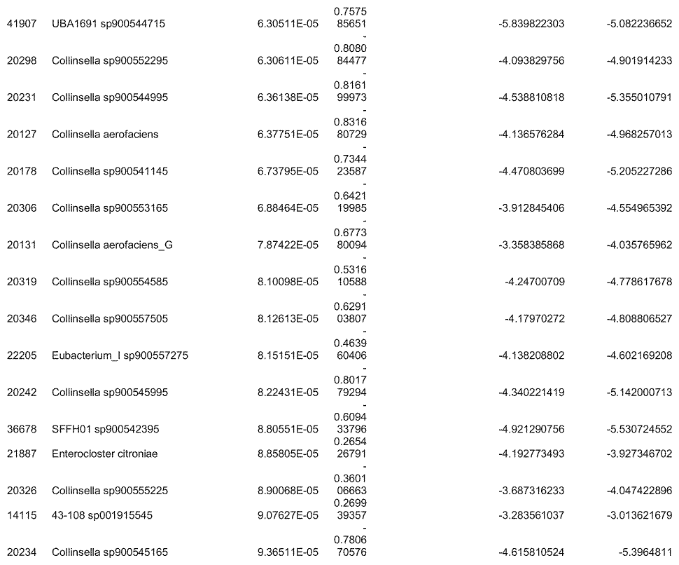

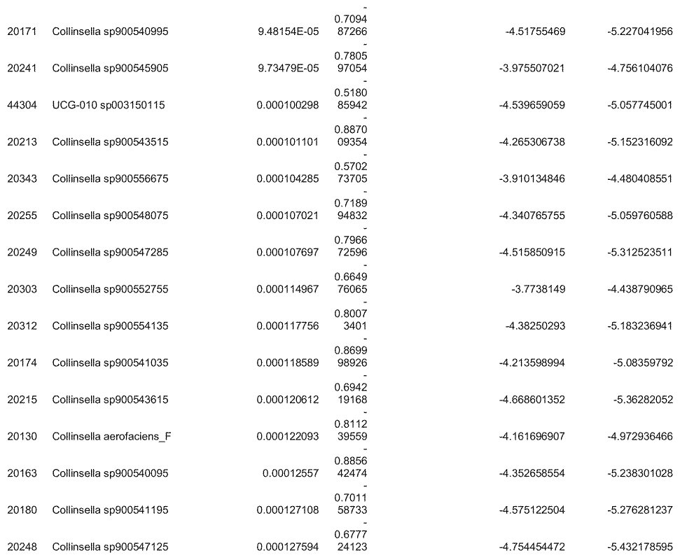

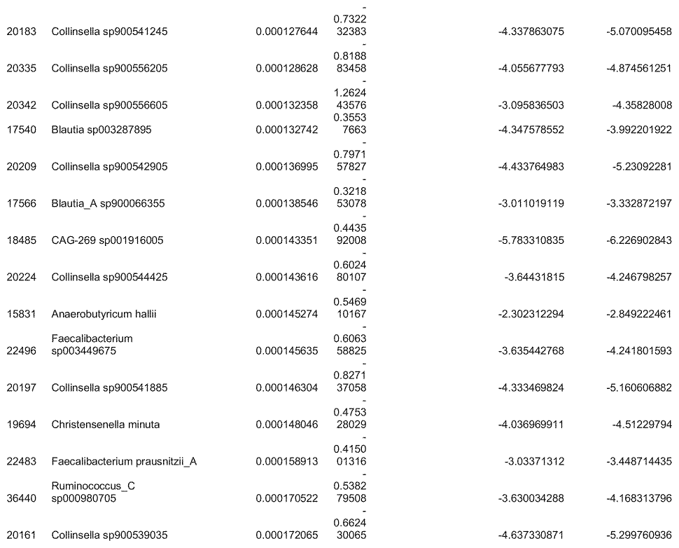

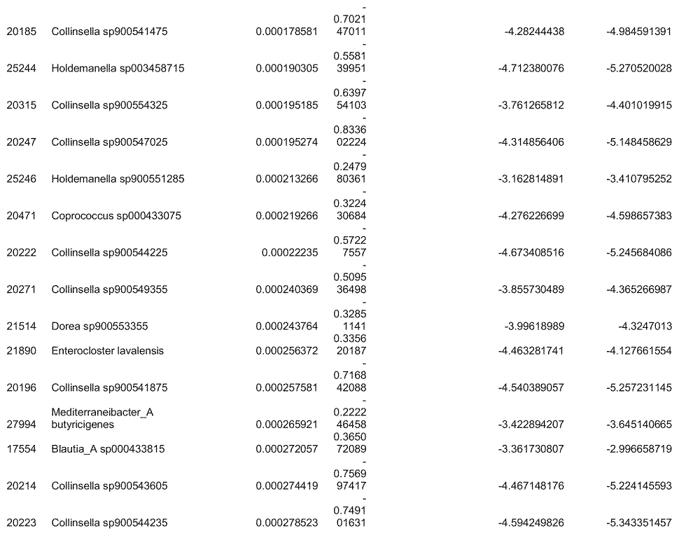

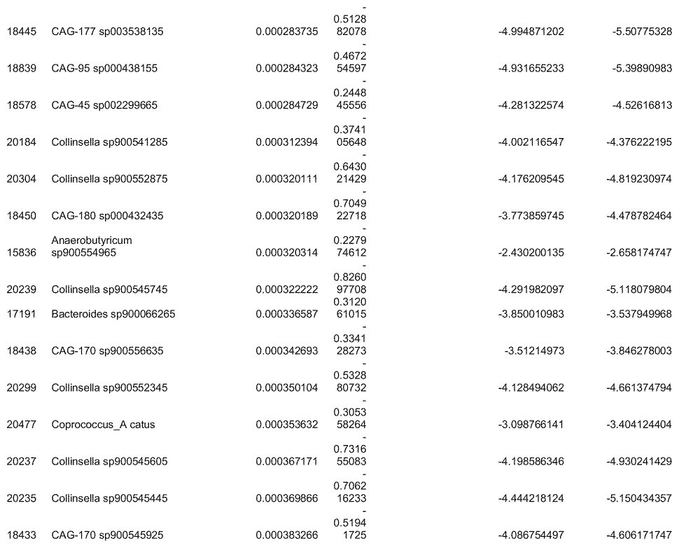

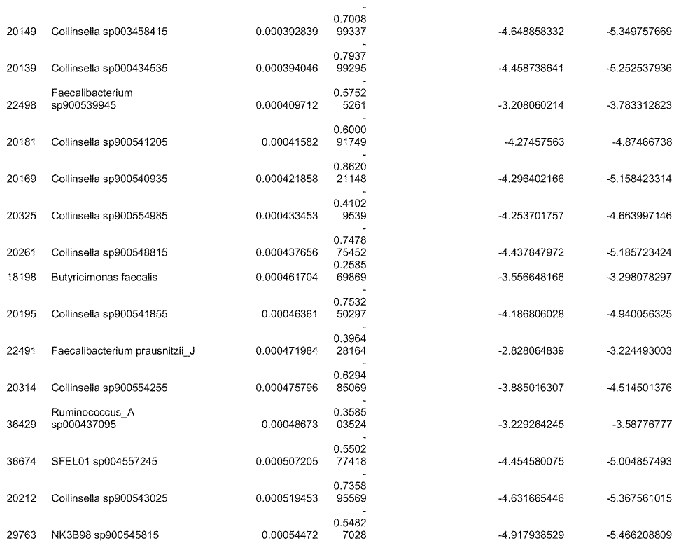

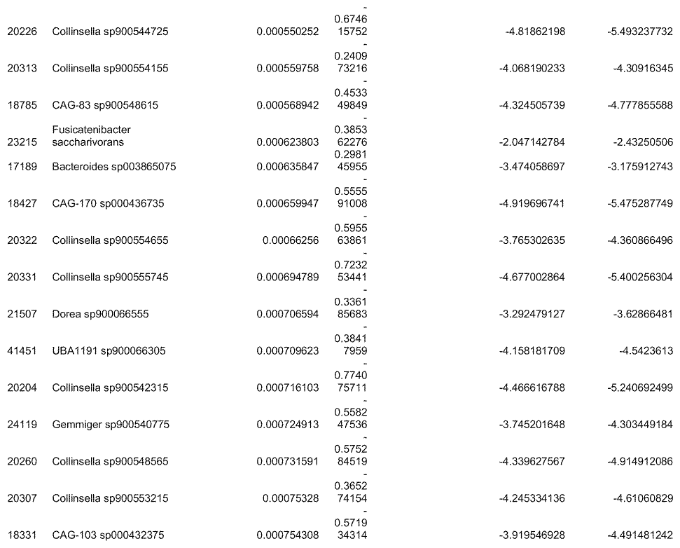

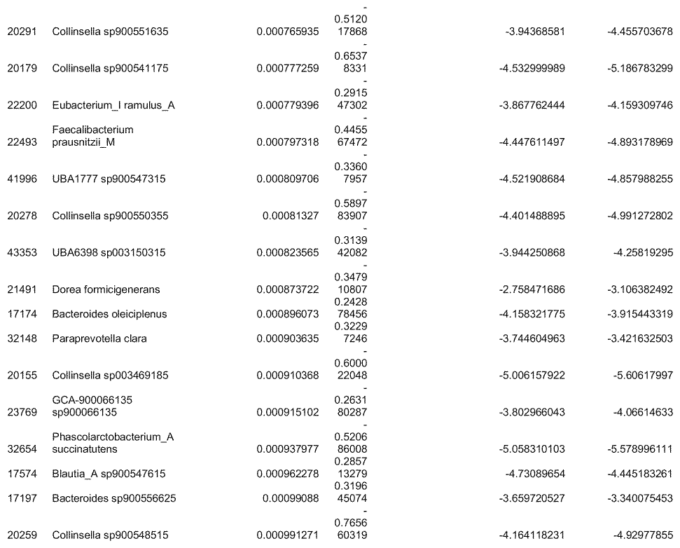

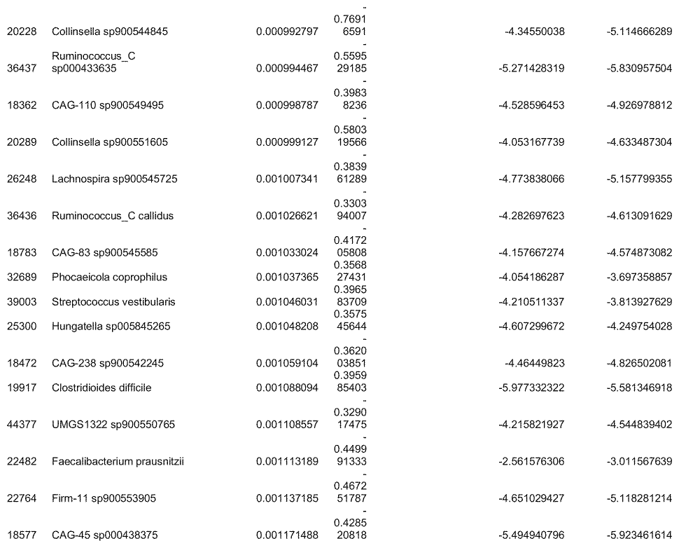

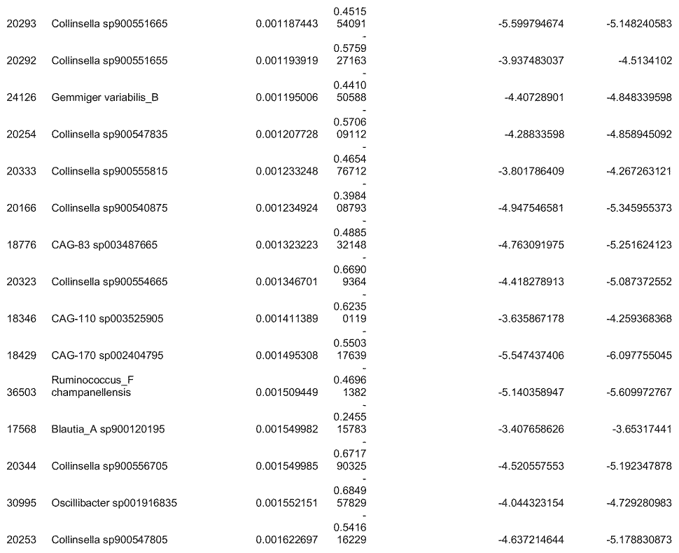

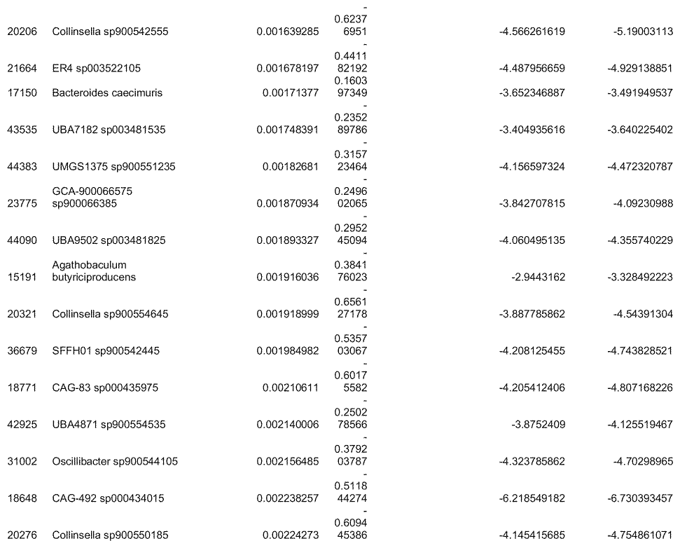

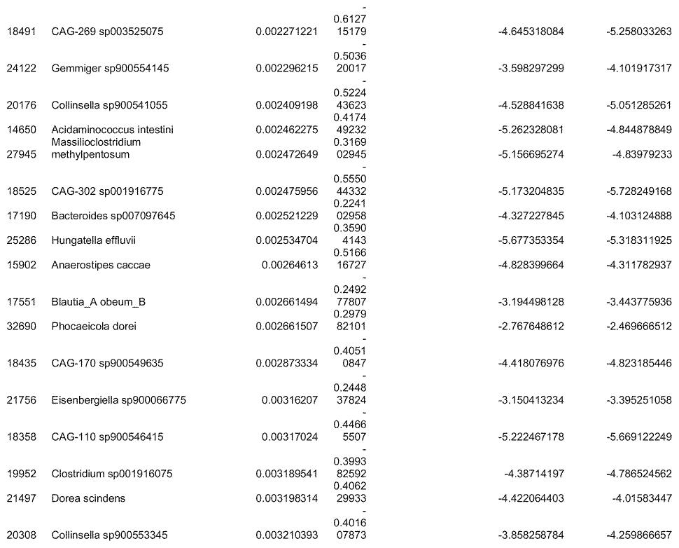

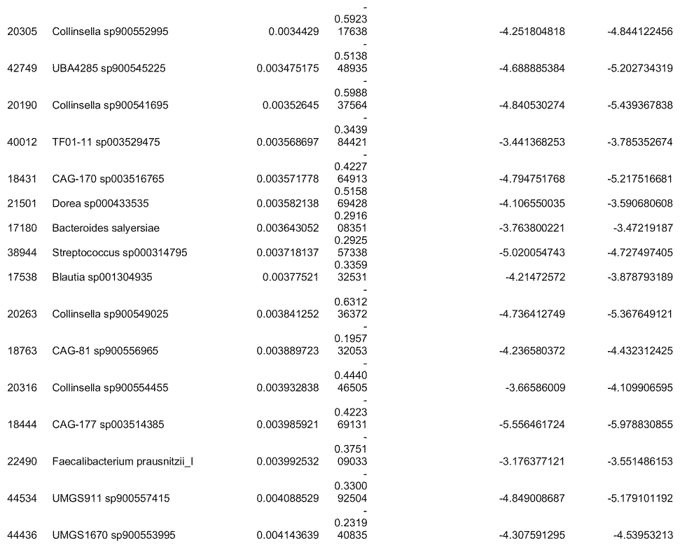

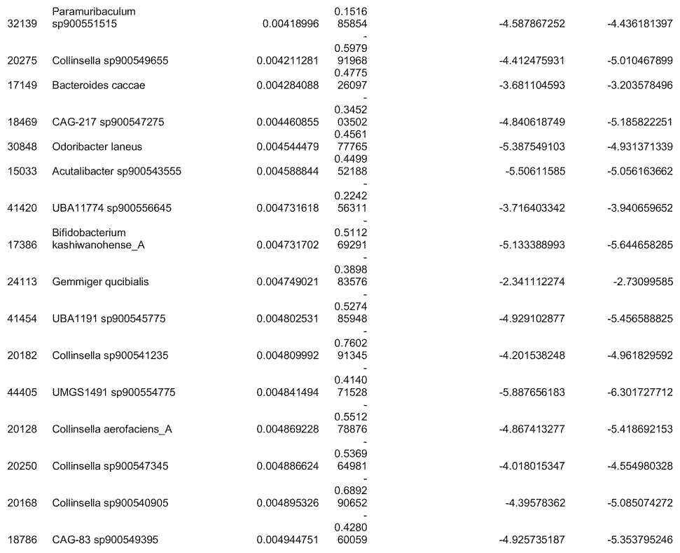

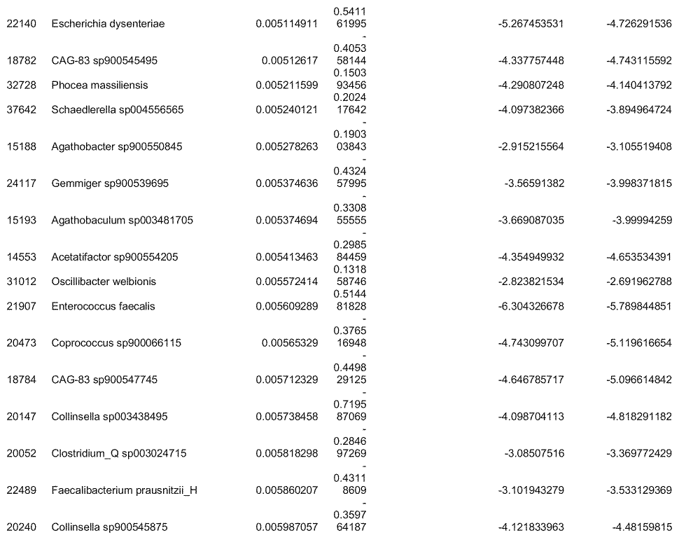

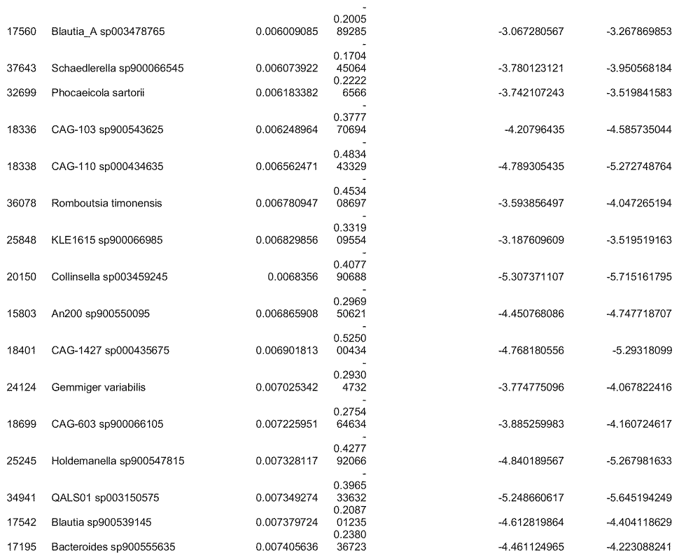

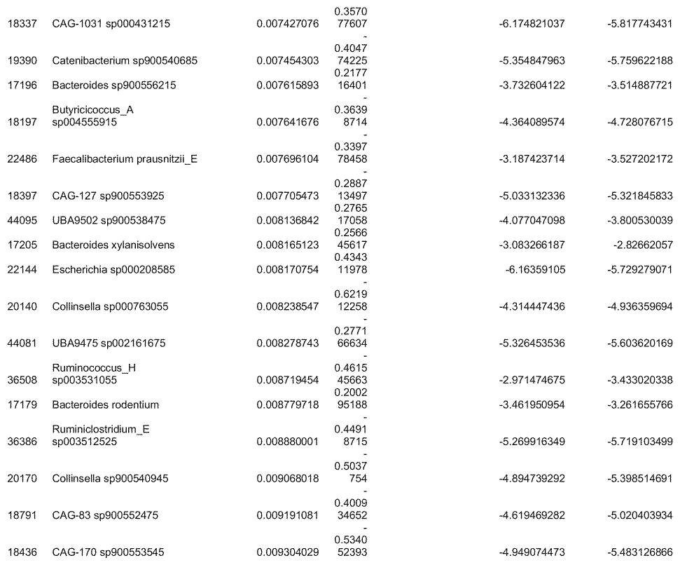

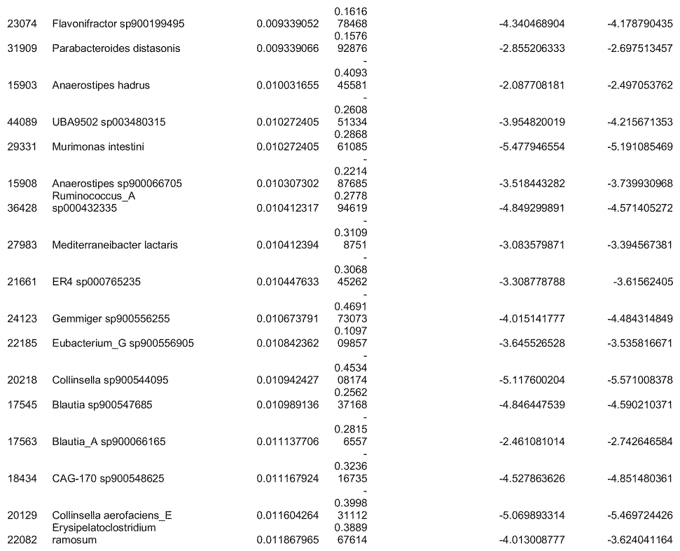

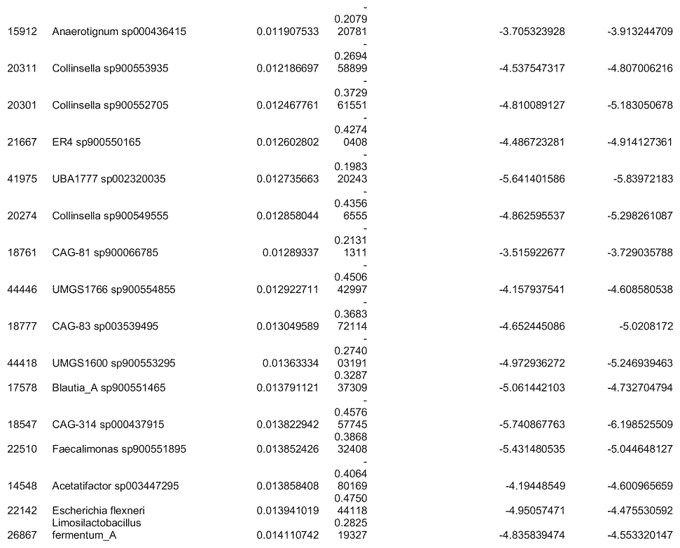

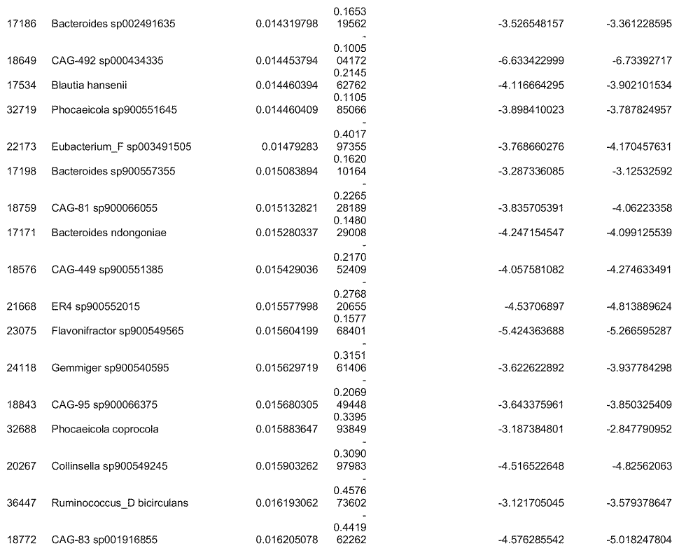

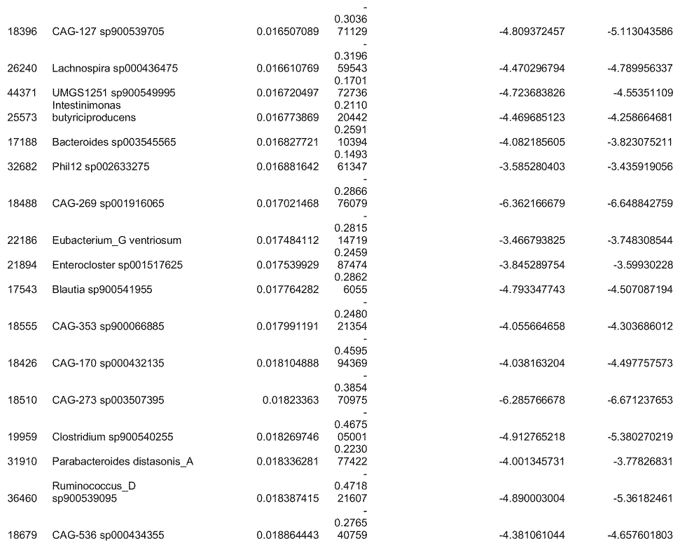

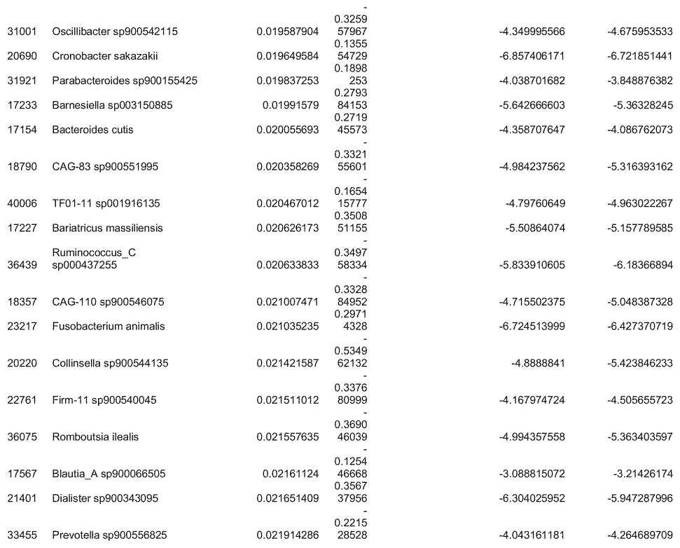

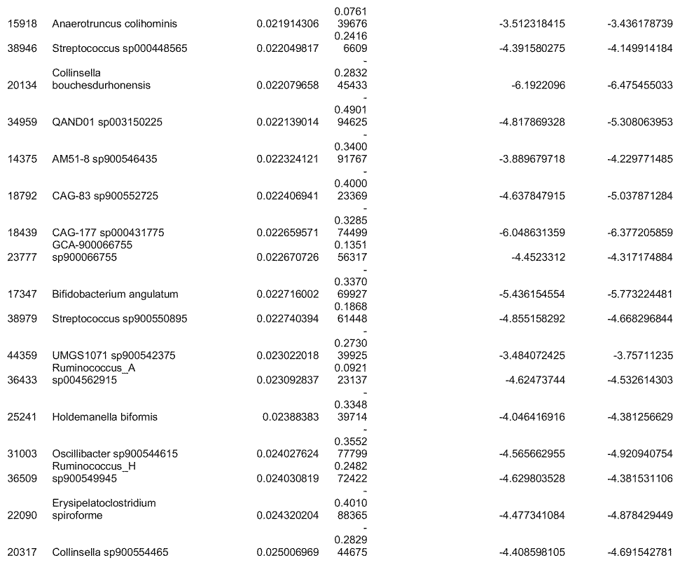

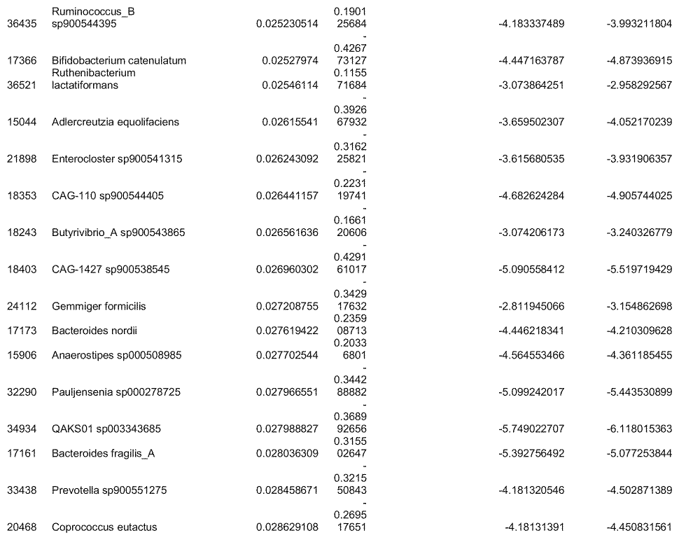

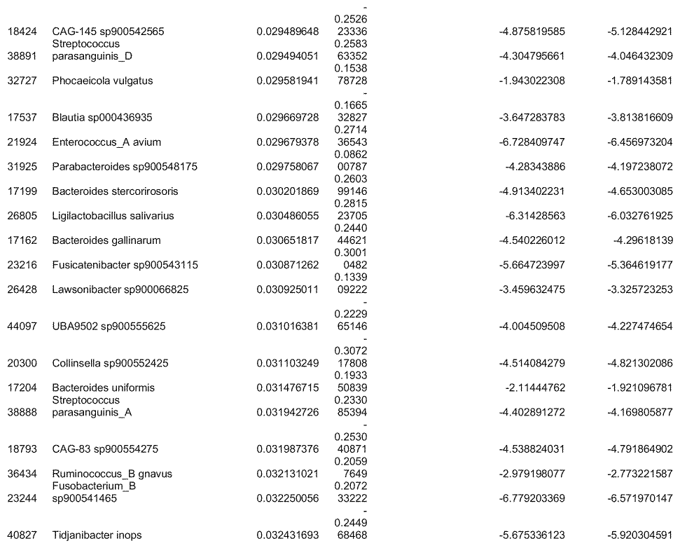

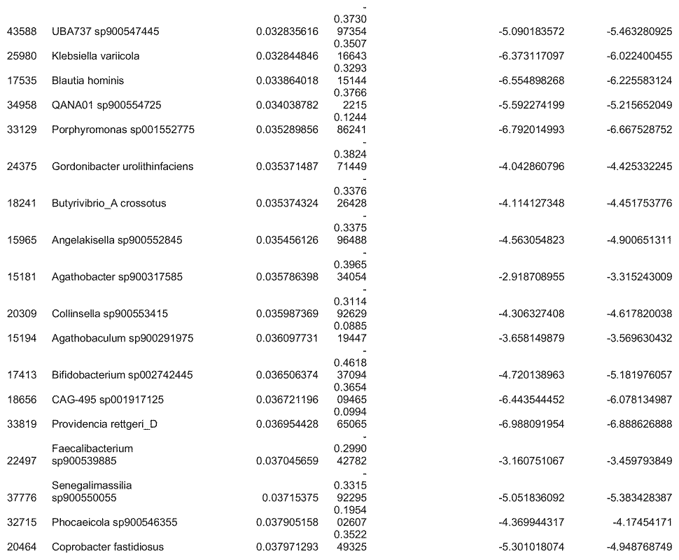

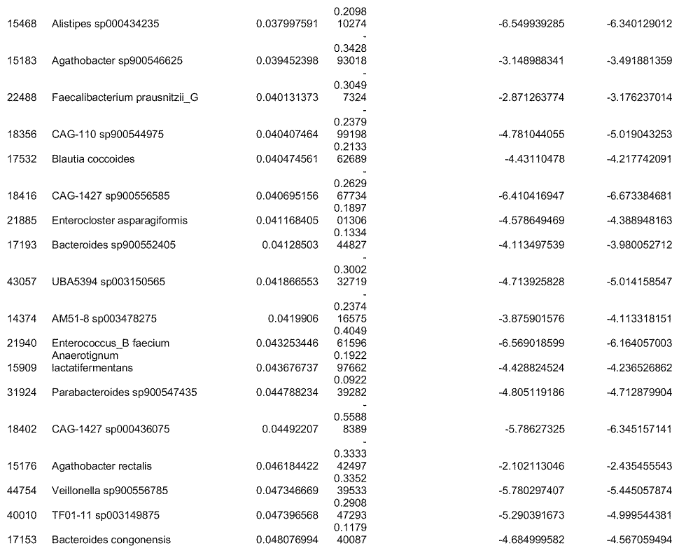

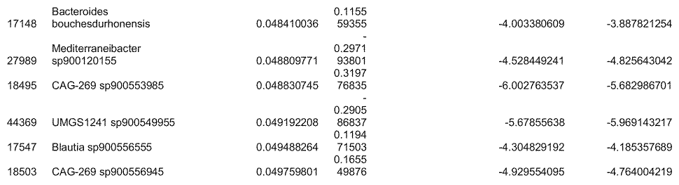

Mice were pre-treated with antibiotics, fecal microbiota transplantation (FMT) was performed, and tumors were inoculated. Randomization and treatment began at a tumor volume of 50 mm³. Tumor size was measured in all animals receiving microbial treatments, antibiotic pre-treatment, followed by FMT transfer from cancer patients with and without anti-CTLA-4, anti-PD1 or anti-PD-L1 therapy. Four FMTs (1-4) were selected for administration to the mice based on donor cancer patient response to therapy. FMTs 1 and 3 are derived from non-responding cancer patients and FMTs 2 and 4 are from cancer patients that respond to immunotherapy. On average, the mice receiving FMTs 1 and 3 from non-responding cancer patients had larger overall tumors than those receiving FMTs 2 and 4 from responding cancer patients, as illustrated in FIG. 47. On average, the animals receiving microbial mix 2 (equal amounts of F. prausnitzii, C. coccoides, R. gnavus, C. scindens, A. muciniphila, and E. hirae) in conjunction with anti-CTLA-4 and FMTs 1 and 3 have a reduction in tumor size compared to those only receiving FMTs 1 and 3 in combination with anti-CTLA-4 as illustrated in FIG. 43. Tumor volume mean and standard error of the mean are displayed for each treatment group, as illustrated in FIG. 44. Tumor volume mean curves and individual tumor sizes plotted as dots are displayed for each treatment group, as illustrated in FIG. 45.

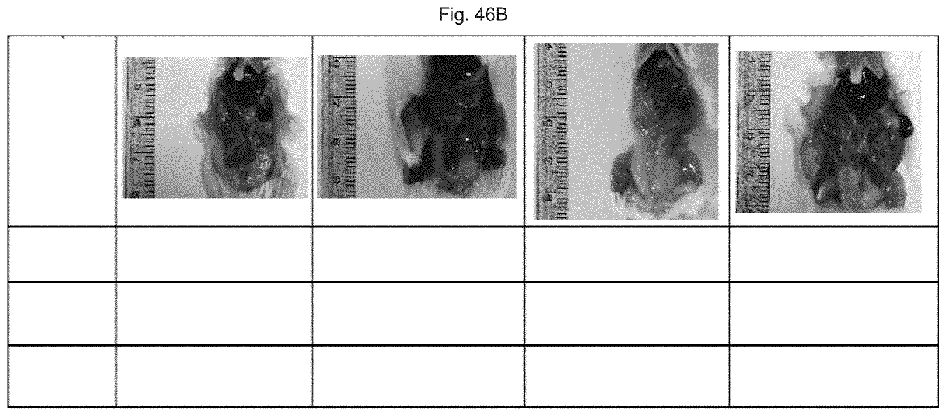

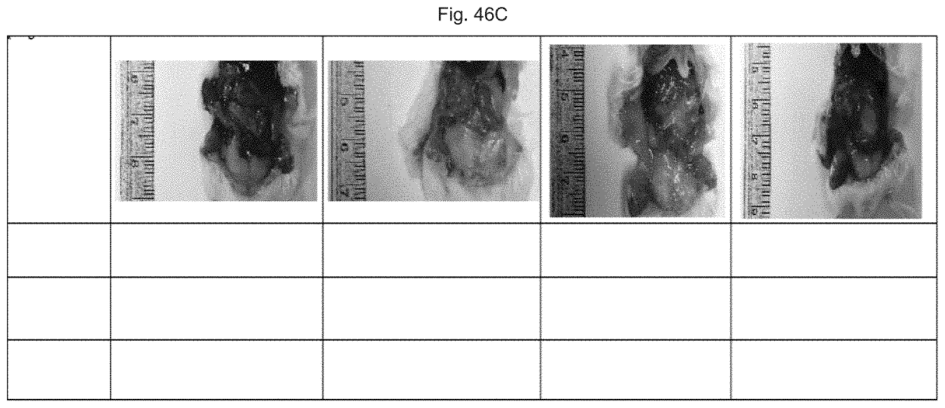

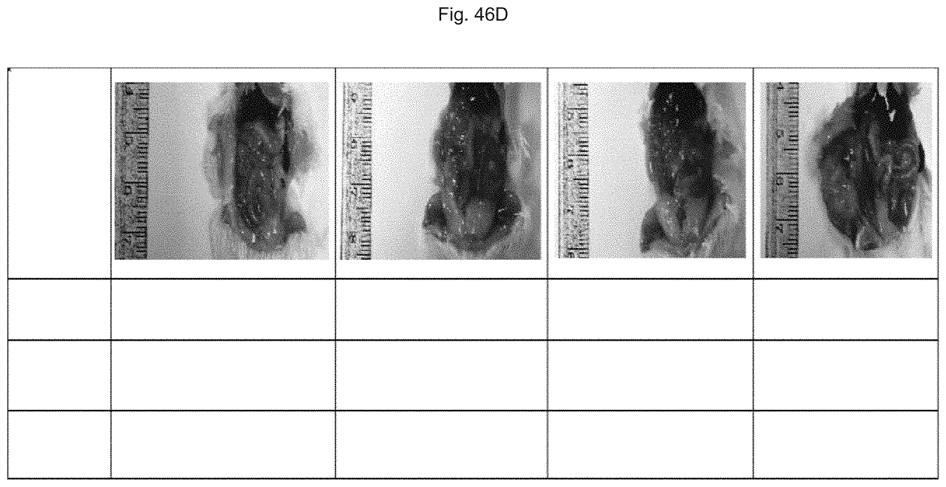

Antibiotic induced depletion of mouse microbiota has been shown to significantly reduce the diversity of the microbiota, gut motility and increase the weight and size of the gastrointestinal tract (Ge et al. J Transl Med (2017) 15:13). Images of the gastrointestinal tract (GI) for mice in both water and antibiotic pre-treatment groups are shown in FIG. 46A-D. The GI tract for antibiotic pre-treatment groups with vehicle or anti-CTLA-4 treatments was enlarged compared to the equivalent water pre-treatment groups. Treatment groups with microbial mix 2 in combination with anti-CTLA-4 and microbial mix 4+ ellagic acid in combination with anti-CTLA-4 had similar sized GI tracts for both pre-treatment groups. The normal size of the GI tract suggests that microbial mixes 2 and 4 have anti-inflammatory properties that may contribute to the observed anti-cancer efficacy.

### Example 18—Efficacy of Live Biotherapeutics as an Antiviral Monotherapy

### Microorganisms in Mouse Study

The sets of microbes to be administered are chosen from either Table 9 (1-294), described in Example 10, and/or Table 42, Example 25, or from engineered microbes described in Examples 12 and 13. Each microbe is isolated from healthy donors, as described in Example 3, or the genetically modified derivatives described in Examples 12 and 13. The live biotherapeutic is cultured and assembled as described in Example 14.

After assembly, PBS-C-G is added to each live biotherapeutic to reduce the total cell density of each live biotherapeutic to the desired dosage level, which can be between 1×10⁸/0.2 ml and 1×10¹²/0.2 ml. Live biotherapeutics are aliquoted into eight 5.0 ml volumes into 15 ml conical tubes and stored at −20° C. until required.

### Mice and Viruses

Several strains of mice including BALB/c, C57BL/6, 129S and transgenic mice (K18-hACE2, A70-hACE2) are obtained from Shanghai Lingchang Biotechnology Co., Ltd (Shanghai, China) or Jackson Laboratory. 6-8-week-old female mice are used. Strains of SARS-CoV, SARS-CoV-2, LCMV, recombinant influenza expressing the LCMV GP33 epitope (PR8-GP33), RSV and A/PR8 (H1N1) viruses are obtained and propagated and titered on Vero E6 cells. Viral titers from 10⁵ to 10⁷ PFU/ml are used. Mice are lightly anesthetized with halothane and infected intranasally with the dosage of virus. Infected mice are examined and weighed daily. To obtain specimens for virus titers, animals are sacrificed, and organs are aseptically removed into sterile phosphate-buffered saline.

### Antibiotic Pre-Treatment

In some studies, mice are treated daily with 200 μL of antibiotic solution via oral gavage for a duration of 1-4 weeks. The antibiotic solution consists of ampicillin (1 mg/mL)(Alfa Aesar J6380706), gentamicin (1 mg/mL)(Acros Organics AC455310050), metronidazole (1 mg/mL)(Acros Organics AC210440050), neomycin (1 mg/mL)(Alfa Aesar AAJ6149922), and vancomycin (0.5 mg/mL) (Alfa Aesar J6279006) via oral gavage. Animals are given at least 48 hours rest period between antibiotic pre-treatment and the treatment phase to allow for antibiotics to go through the system.

### Fecal Microbiota Transplantation (FMT)

Fecal Microbiota Transplantation (FMT) of a human gut microbiome into antibiotic treated mice is a method for standardizing microbiome composition. FMT is performed in some experiments with fecal material derived from healthy donors, donors infected with viruses and responders to checkpoint inhibitor therapy (R) or non-responders to checkpoint inhibitor therapy (NR). Not only does this standardize the mice microbiomes, but also conditions them to favor response or non-response, respectively. Following antibiotic pre-treatment, colonization is performed by oral gavage with 200 μl of suspension obtained by homogenizing the fecal samples in PBS. Mouse fecal samples are collected 1-2 times during this period, so that the efficacy of the FMT can be evaluated. Following FMT, a rest period of 5-7 days is allowed to pass prior to treatment initiation.

### Histology and Immunohistochemistry

Organs are harvested from infected and uninfected mice and fixed in zinc formalin. For histology, sections are stained with hematoxylin and eosin. To detect viral antigen, sections are probed with a monoclonal antibody (MAb) to the SARS-CoV and SARS-CoV-2 N protein (Zymed, San Francisco, Calif.), or any viral antigen or a control immunoglobulin G2a Mab (E-Bioscience, San Diego, Calif.) followed by a biotinylated goat anti-mouse secondary antibody (1:200; Jackson Immunoresearch, West Grove, Pa.). Samples are developed by sequential incubation with a streptavidin-horseradish peroxidase conjugate (Jackson Immunoresearch) and diaminobenzidine (Sigma-Aldrich).

### Peripheral Blood Extraction and Processing

Whole blood is taken via cardiac puncture at the end of the experiment, or via tail bleed during the experiment, and collected into an EDTA tube. Plasma is isolated from an aliquot of the whole blood by centrifugation at 1500×g for 10 minutes, taking the supernatant. A second centrifugation is performed to remove any residual blood cells.

Peripheral blood mononuclear cells (PBMCs) are isolated from blood using a standard kit and stored in liquid nitrogen at 1×10⁶ cells/mL until use. Prior to storage, PBMC's may be processed using flow sorting or antibody spin separation kit to select for a certain purified lymphocyte subpopulation, such as T cells.

### GI Tract Removal and Analysis

After mice are euthanized at the termination of the study, the intact digestive tract of each mouse from stomach to rectum are removed and kept in a 5 ml Eppendorf tube on ice prior to dissection. Forceps are sterilized by soaking in 100% ethanol and then used to remove the intestine length and stretch it on a work surface covered with cellophane. With the use of ethanol-sterilized dissection scissors, 3 cm lengths of the jejunum nearest to the stomach and the ilium nearest to the cecum/large intestine are excised and then each placed with forceps in a 1.5 ml Eppendorf tube and placed on ice. A 2 cm segment of the cecum/ascending colon is then excised, as are 2 cm segments of the transcending colon and the descending colon, and all are placed in 1.5 ml Eppendorf tubes on ice. Dissection instruments are sterilized by dipping in 100% ethanol between each intestine fragment removal. To each tube containing dissected intestinal segments is added 0.5 ml ice cold PBS buffer. A plastic pestle is used to press and massage the intestinal segment in each tube to expel ruminal matter, which is then removed by pipette and placed in a fresh Eppendorf tube. Tubes containing expelled ruminal matter from each intestinal segment are immediately placed on dry ice and then stored for later analyses at −80° C. Remaining intestinal tissues are then rinsed twice by adding and then removing 0.5 ml ice cold PBS. Rinsed intestinal fragment tissues are then frozen on dry ice and then stored at −80° C. for later analysis.

### Analyses of Dendritic Cell Subsets in Treated Mice

Cell suspensions from mouse spleen and lymph nodes are prepared by digestion with collagenase and DNase for 60 min and subsequently strained through a 70 mm mesh. Colonic and small intestinal lymphocytes are isolated as previously described (Viaud, S. et al. Science 80(342): 971-976 (2013). In brief, cecum, colon and small intestine are digested in PBS containing 5 mM EDTA and 2 mM DTT shaking at 37° C. A plastic pestle is used to press and massage the intestinal segment in each tube to expel ruminal matter, which is then removed by pipette and placed in a fresh Eppendorf tube. Tubes containing expelled ruminal matter from each intestinal segment are immediately placed on dry ice and then stored for later analyses at −80° C. Remaining intestinal tissues are then rinsed twice by adding and then removing 0.5 ml ice cold PBS. Rinsed intestinal fragment tissues are then frozen on dry ice in RNALater (Thermo Fisher Scientific) and then stored at −80° C. for later analysis.

After initial digestion colonic and small intestinal tissue pieces are digested in collagenase/Dnase containing RPMI medium for 30 min. Tissue pieces are further strained through a 70 mm mesh. For flow cytometry analyses, cell suspensions are stained with antibodies against the following surface markers: CD11c (N418), CD11b (M1/70), Ly6c (HK1.4), MHC class II (M5/114.15.2), CD24 (M1/69), CD64 (X54-5/7.1), CD317 (ebio927), CD45 (30-F11), F4/80 (C1:A3-1), CD8a (53-6.7). DAPIis used for dead cell exclusion. Antibodies are purchased from eBiosciences, BD Biosciences or BioLegend respectively. Cell populations are gated as follows: small intestine (migratory fraction): CD103+DC (CD45+CD11c+MHC-II+CD103+CD24+), CD11b+CD103+(CD45+CD11c+MHC-II+CD103+CD11b+CD24+), CD11b+(CD45+CD11c+MHC-II+CD11b+CD24+), inflammatory DC (CD45+CD11c+MHC-II+CD11b+CD64+Ly6c+), large intestine: CD103+DC (CD45+CD11c+MHC-II+CD103+CD24+), CD11b+(CD45+CD11c+MHC-II+CD11b+CD24+), inflammatory DC (CD45+CD11c+MHC-II+CD11b+CD64+Ly6c+).

### Whole Genome Sequencing

Fecal gDNA is extracted for whole genome sequencing (WGS). Experimental methods for DNA extraction and library preparation are performed using protocols modeled after the Human Microbiome Project (Lloyd-Price et al. (2017) Nature 550(7674):61-66) and validated with samples from healthy volunteers. Sequencing is performed by an outside service provider, using a HISEQ-X® (Illumina) with 2×150 bp paired-end reads, providing approximately 4 million reads per sample. Analysis software such as Centrifuge (Kim, D., et al., Centrifuge: rapid and sensitive classification of metagenomic sequences. Genome Res, 2016. 26(12): p. 1721-1729) are used to align sequence reads to reference genomes and obtain species and strain-level identification.

### Metabolomics

Metabolites are extracted from fecal material or blood plasma, using methanol under vigorous shaking for 2 min (Glen Mills GenoGrinder 2000) to precipitate protein and dissociate small molecules bound to protein or trapped in the precipitated protein matrix, followed by centrifugation to recover chemically diverse metabolites. The resulting extract was divided into five fractions: two for analysis by two separate reverse phase (RP)/UPLC-MS/MS methods using positive ion mode electrospray ionization (ESI), one for analysis by RP/UPLC-MS/MS using negative ion mode ESI, one for analysis by HILIC/UPLC-MS/MS using negative ion mode ESI, and one reserved for backup. Samples are placed briefly on a TurboVap® (Zymark) to remove the organic solvent, followed by injection on one of the instruments mentioned above. Compounds are identified by comparison to library entries of purified standards, that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library+/−10 ppm, and the MS/MS forward and reverse scores. MS/MS scores are based on a comparison of the ions present in the experimental spectrum to ions present in the library entry spectrum. While there may be similarities between these molecules based on one of these factors, the use of all three data points can be utilized to distinguish and differentiate biochemicals. Peaks are quantified as area-under-the-curve detector ion counts.

### Immunophenotyping Assays

Immune profiling of whole blood is utilized to assess T cell activation in response to microbial treatment. In some experiments, immune phenotyping is also performed on tissue obtained from the GI tract.

For flow cytometry analysis, 1 mL of RBC Lysis Buffer is added to 0.1 mL of whole blood or homogenized tissue and allowed to incubate at room temperature for 10 minutes. Lysis is quenched by adding 10 mL of cold DPBS. Samples are centrifuged at 1500 rpm for 5 minutes at 4° C. The pellet is aspirated and resuspend in another 10 mL of cold DPBS. Samples are recentrifuged at 1500 rpm for 5 minutes at 4° C. Samples are resuspended in 500 μL of FACS buffer and transferred to a 96-well plate. Samples are stained with Fixable Viability ef780™ (eBioscience), CD45-PEcy7 (BioLegend), CD3-BV605™ (BioLegend), CD8-AF700™ (BioLegend), and CD4-AF488™ (BioLegend). Stained samples are run on a BD LSRFortessa™ flow cytometer and analyses are performed with FlowJo™ (Tree Star).

Alternatively, CyTOF® is applied to characterize the immune profile of the PBMCs. This work is conducted by the Bioanalytical and Single-Cell Facility at the University of Texas, San Antonio, and entails a comprehensive panel of 29 different immune markers, allowing for deep interrogation of cellular phenotype and function (https://www.fluidigm.com/products/helios). To complement these results, RNA sequencing is applied to the entire population of the PBMCs, sorted populations, and also to single cells. Single cell RNAseq is applied using the method developed by 10×Genomics (https://www.10xgenomics.com/solutions/single-cell/). Finally, cytokine levels are determined using the Human Cytokine 30-Plex Luminex assay (https://www.thermofisher.com/order/catalog/product/LHC6003M).

### Example 19—Therapeutic Effect of Microbes on Efficacy of Antiviral Therapy

In this study, live biotherapeutics as provided herein, including combinations of microbes as provided herein, are administered in combination with antiviral therapies (a small molecule, a vaccine, an antibody, a cell therapy, a natural killer (NK) cell therapy, angiotensin II receptor blockers, a defensin-mimetic, a nanobody, a peptide, an immune modulator, an immunotherapy, an anti-necrosis, a nucleoside, a quinoline compound, a protease inhibitor, a sphingosine kinase-2 (SK2) inhibitor, an interleukin receptor antagonist and nanoviricide) to demonstrate the ability of these microbes to enhance antiviral immunity.

### Microorganisms in Mouse Study

The sets (or combinations) of microbes to be administered are chosen from the list of exemplary bacterial combinations as set forth in Table 9, listing combinations 1 to 294, as described in Example 10, or from the exemplary engineered microbes described in Examples 12 and 13, or from Table 42, Example 25. Each microbe is isolated from healthy donors, as described in Example 3, or the genetically modified derivatives described in Examples 12 and 13. The live biotherapeutic is cultured and assembled as described in Example 14.

After assembly, PBS-C-G is added to each microbial mix to reduce the total cell density of each microbial mix to the desired dosage level, which can be between 1×10^{8/}0.2 ml and 1×10¹²/0.2 ml. Live biotherapeutics are aliquoted into eight 5.0 ml volumes into 15 ml conical tubes and stored at −20° C. until required.

### Mice and Viruses

Several strains of mice including BALB/c, C57BL/6, 129S and transgenic mice (K18-hACE2, A70-hACE2) are obtained from Shanghai Lingchang Biotechnology Co., Ltd (Shanghai, China) or Jackson Laboratory. 6-8-week-old female mice are used. Strains of SARS-CoV, SARS-CoV-2, LCMV, recombinant influenza expressing the LCMV GP33 epitope (PR8-GP33) and A/PR8 (H1N1) viruses are obtained and propagated and titered on Vero E6 cells. Viral titers from 10⁵ to 10⁷ PFU/ml are used. Mice are lightly anesthetized with halothane and infected intranasally with the dosage of virus. Infected mice are examined and weighed daily. To obtain specimens for virus titers, animals are sacrificed, and organs are aseptically removed into sterile phosphate-buffered saline.

### Antibiotic Pre-Treatment

In some studies, mice are treated daily with 200 μL of antibiotic solution via oral gavage for a duration of 1-2 weeks. The antibiotic solution consists of ampicillin (1 mg/mL)(Alfa Aesar J6380706), gentamicin (1 mg/mL)(Acros Organics AC455310050), metronidazole (1 mg/mL)(Acros Organics AC210440050), neomycin (1 mg/mL)(Alfa Aesar AAJ6149922), and vancomycin (0.5 mg/mL) (Alfa Aesar J6279006) via oral gavage. Animals are given at least 48 hrs rest period between antibiotic pre-treatment and the treatment phase to allow for antibiotics to go through system.

### Fecal Microbiota Transplantation (FMT)

In alternative embodiments, methods as provided herein comprise use of Fecal Microbiota Transplantation (FMT), or elements used to practice FMT, as described for example, in U.S. Pat. Nos. 10,493,111; 10,463,702; 10,383,519; 10,369,175; 10,328,107.

FMT of a human gut microbiome into antibiotic treated mice is a method for standardizing microbiome composition. FMT is performed in some experiments with fecal material derived from responders to checkpoint inhibitor therapy (R) or non-responders to checkpoint inhibitor therapy (NR). Not only does this standardize the mice microbiomes, but also conditions them to favor response or non-response, respectively. Following antibiotic pre-treatment, colonization is performed by oral gavage with 200 μl of suspension obtained by homogenizing the fecal samples in PBS. Mouse fecal samples are collected 1-2 times during this period, so that the efficacy of the FMT can be evaluated. Following FMT, a rest period of 5-7 days can pass prior to treatment initiation.

### Histology and Immunohistochemistry

Organs are harvested from infected and uninfected mice and fixed in zinc formalin. For histology, sections are stained with hematoxylin and eosin. To detect viral antigen, sections are probed with a monoclonal antibody (MAb) to the SARS-CoV and SARS-CoV-2 N protein (Zymed, San Francisco, Calif.), or any viral antigen or a control immunoglobulin G2a Mab (E-Bioscience, San Diego, Calif.) followed by a biotinylated goat anti-mouse secondary antibody (1:200; Jackson Immunoresearch, West Grove, Pa.). Samples are developed by sequential incubation with a streptavidin-horseradish peroxidase conjugate (Jackson Immunoresearch) and diaminobenzidine (Sigma-Aldrich).

### Peripheral Blood Extraction and Processing

Whole blood is taken via cardiac puncture at the end of the experiment, or via tail bleed during the experiment, and collected into an EDTA tube. Plasma is isolated from an aliquot of the whole blood by centrifugation at 1500×g for 10 minutes, taking the supernatant. A second centrifugation is performed to remove any residual blood cells.

Peripheral blood mononuclear cells (PBMCs) are isolated from blood using a standard kit and stored in liquid nitrogen at 1×10⁶ cells/mL until use. Prior to storage, PBMC's may be processed using flow sorting or antibody spin separation kit to select for a certain purified lymphocyte subpopulation, such as T cells.

### GI Tract Removal and Analysis

After mice are euthanized at the termination of the study, the intact digestive tract of each mouse from stomach to rectum are removed and kept in a 5 ml Eppendorf tube on ice prior to dissection. Forceps are sterilized by soaking in 100% ethanol and then used to remove the intestine length and stretch it on a work surface covered with cellophane. With the use of ethanol-sterilized dissection scissors, 3 cm lengths of the jejunum nearest to the stomach and the ilium nearest to the cecum/large intestine are excised and then each placed with forceps in a 1.5 ml Eppendorf tube and placed on ice. A 2 cm segment of the cecum/ascending colon is then excised, as are 2 cm segments of the transcending colon and the descending colon, and all are placed in 1.5 ml Eppendorf tubes on ice. Dissection instruments are sterilized by dipping in 100% ethanol between each intestine fragment removal. To each tube containing dissected intestinal segments is added 0.5 ml ice cold PBS buffer. A plastic pestle is used to press and massage the intestinal segment in each tube to expel ruminal matter, which is then removed by pipette and placed in a fresh Eppendorf tube. Tubes containing expelled ruminal matter from each intestinal segment are immediately placed on dry ice and then stored for later analyses at −80° C. Remaining intestinal tissues are then rinsed twice by adding and then removing 0.5 ml ice cold PBS. Rinsed intestinal fragment tissues are then frozen on dry ice and then stored at −80° C. for later analysis.

### Analyses of Dendritic Cell Subsets in Treated Mice

Cell suspensions from mouse spleen and lymph nodes are prepared by digestion with collagenase and DNase for 60 min and subsequently strained through a 70 mm mesh. Colonic and small intestinal lymphocytes are isolated as previously described (Viaud, S. et al. Science (80-.). 342, 971-976 (2013). In brief, cecum, colon and small intestine are digested in PBS containing 5 mM EDTA and 2 mM DTT shaking at 37° C. A plastic pestle is used to press and massage the intestinal segment in each tube to expel ruminal matter, which is then removed by pipette and placed in a fresh Eppendorf tube. Tubes containing expelled ruminal matter from each intestinal segment are immediately placed on dry ice and then stored for later analyses at −80° C. Remaining intestinal tissues are then rinsed twice by adding and then removing 0.5 ml ice cold PBS. Rinsed intestinal fragment tissues are then frozen on dry ice in RNALater (Thermo Fisher Scientific) and then stored at −80° C. for later analysis.

After initial digestion colonic and small intestinal tissue pieces are digested in collagenase/DNase containing RPMI medium for 30 min. Tissue pieces are further strained through a 70 mm mesh. For flow cytometry analyses, cell suspensions are stained with antibodies against the following surface markers: CD11c (N418), CD11b (M1/70), Ly6c (HK1.4), MHC class II (M5/114.15.2), CD24 (M1/69), CD64 (X54-5/7.1), CD317 (ebio927), CD45 (30-F11), F4/80 (C1:A3-1), CD8a (53-6.7). DAPIis used for dead cell exclusion. Antibodies are purchased from eBiosciences, BD Biosciences or BioLegend respectively. Cell populations are gated as follows: small intestine (migratory fraction): CD103+DC (CD45+CD11c+MHC-II+CD103+CD24+), CD11b+CD103+(CD45+CD11c+MHC-II+CD103+CD11b+CD24+), CD11b+(CD45+CD11c+MHC-II+CD11b+CD24+), inflammatory DC (CD45+CD11c+MHC-II+CD11b+CD64+Ly6c+), large intestine: CD103+DC (CD45+CD11c+MHC-II+CD103+CD24+), CD11b+(CD45+CD11c+MHC-II+CD11b+CD24+), inflammatory DC (CD45+CD11c+MHC-II+CD11b+CD64+Ly6c+).

### Analysis of Fecal and Blood Samples

Whole genome sequencing, metabolomics, and immunophenotyping are performed on samples collected, as described in Example 15.

### Example 20: Method of Treating a Subject with a Live Exemplary Biotherapeutic

This example describes administration of a live exemplary biotherapeutic as provided herein, including a combination of bacteria as provided herein, for example, as set forth in Table 9, Example 10, and/or Table 42, Example 25, to an individual in need thereof.

A patient is suffering from a viral infection, such as that caused by SARS-CoV-2 or other coronaviruses, or any influenza virus. The patient is administered live biotherapeutic compositions, i.e., a formulation or a pharmaceutical composition comprising a combination of microbes (for example, bacteria) as provided herein, (Table 9, and as described in Example 10, and/or Table 42, Example 25) either in monotherapy or in combination with a reverse transcriptase inhibitor, protease inhibitor, integrase inhibitor, fusion inhibitor, chemokine receptor antagonist, cell therapy, immunotherapy, or any other antiviral treatment, or a vaccine, and the patient can be administered the live biotherapeutic for the duration of treatment or for only one or several segments of treatment.

In alternative embodiments, each or one of the microbes used in the bacterial combination is (at least initially) isolated from a healthy donor or donors, as described in Example 3, or is a genetically modified derivative as described in Examples 12 and 13, or is a cultured derivative either.

In alternative embodiments, the patient is administered a live biotherapeutic at a dose of between about 10⁵ to 10¹⁵ bacteria, or at a dose of about 10¹⁰, 10¹¹ or 10¹² bacteria total or per dose, which can be in a lyophilized form, for example, or formulated in an enteric coated capsule. In alternative embodiments, the patient takes 1, 2, 3, 4, 5, 6, 7, 8, 9, 10 or 11 or more live biotherapeutic capsules (for example, by mouth or suppository) once, twice or three times or more per day, and the patient can resume a normal diet after about 1, 2, 4, 8, 12, or 24 or more hours.

In another embodiment, the patient may take the live biotherapeutic capsule(s) by mouth before, during, and/or immediately after a meal.

In another embodiment, the patient is given a course of antibiotics before treatment, for example, between one to seven days, or between about one to two weeks prior to the first dose of the live biotherapeutic (for example, as capsule(s)).

In another embodiment, dosing of the live biotherapeutic, for example, as capsule(s), is started one to seven days prior to administration of a first dose of antiviral treatment.

In another embodiment, dosing of the live biotherapeutic capsule(s) is continued 1 month, 6 months, 1 year, or more, or between about one week and 2 years, following termination of antiviral treatment or full recovery from disease.

In alternative embodiments, severity of the disease and patient response to the therapy can be scored based on time to viral clearance, time to symptom-free recovery, number of days with high fever, or severity of symptoms.

### Example 21: Method of Treating a Subject with an Exemplary Live Biotherapeutic Based on Stool Biomarkers

This example describes administration of a live exemplary biotherapeutic as provided herein, including a combination of bacteria as provided herein, for example, as set forth in Table 9, Example 10, and/or Table 42, Example 25, to an individual in need thereof.

A patient is suffering from a viral infection, such as that caused by SARS-CoV-2 or other coronaviruses, or any influenza virus. The patient's stool is collected and analyzed using the methods described in Example 9. In one embodiment, whole genome sequencing is performed and the presence of microbes that are characteristic of patients with less severe symptoms and faster recovery is evaluated. The complete organism abundance profile is also plotted on the PCA axes shown in FIG. 3. Based on the abundance profiles of test subjects, a classifier is developed to predict if any given microbiome composition represents someone who recovers well from the viral infection or someone who has poor recovery. This may be based on the amount of one or more particular organisms present, position in the PCA plot, or other criteria that combines aspects of the whole genome sequence data. This classifier is applied to the patient's microbiome composition to determine if the patient is at risk for severe symptoms.

In another embodiment, metabolomics is performed on the stool or plasma; a classifier is developed based concentrations of one or more metabolites in all patient data collected to date, and the patient prognosis is predicted based on this classification.

If the patient is classified as at risk, a live biotherapeutic will be administered to change the microbiome to be more like that of someone who recovers quickly. The patient is administered one of the present live biotherapeutics (Table 9, and as described in Example 10, and/or Table 42, Example 25) in combination with a reverse transcriptase inhibitor, protease inhibitor, integrase inhibitor, fusion inhibitor, chemokine receptor antagonist, or any other antiviral treatment, and the patient can be administered the live biotherapeutic for the duration of treatment. Each microbe is isolated from healthy donors, as described in Example 3, or the genetically modified derivatives described in Examples 12 and 13.

In alternative embodiments, the patient is administered a live biotherapeutic at a dose of between about 10⁵ to 10¹⁵ bacteria, or at a dose of about 10¹⁰, 10¹¹ or 10¹² bacteria total or per dose, which can be in a lyophilized form, for example, formulated in an enteric coated capsule. The patient takes 1, 2, 3, 4, 5, 6, 7, 8, 9, 10 or 11 or more live biotherapeutic capsules by mouth once, twice or three times per day, and resumes a normal diet after 2, 4, 8, 12, or 24 hours.

In another embodiment, the patient takes the capsule by mouth before, during, or immediately after a meal.

In another embodiment, the patient is given a course of antibiotics before treatment, for example, between one to seven days, or between about one to two weeks prior to the first dose of microbial cocktail.

In another embodiment, dosing of the live biotherapeutic capsule(s) is continued 1 month, 6 months, 1 year, or more, or between about one week and 2 years, following termination of antiviral treatment or full recovery from disease.

In alternative embodiments, severity of the disease and patient response to the therapy can be scored based on time to viral clearance, time to symptom-free recovery, number of days with high fever, or severity of symptoms.

### Example 22: Diagnosis of Disease and Method of Treating a Subject with an Exemplary Microbial Therapeutic

This example describes administration of a live exemplary biotherapeutic as provided herein, including a combination of bacteria as provided herein, for example, as set forth in Table 9, Example 10, and/or Table 42, Example 25, to an individual in need thereof.

Stool biomarkers based on microbes present in patients that recover quickly from viral infections, that are also lacking in patients that have severe symptoms or recover from the infection slowly, can be used to predict the composition of live biotherapeutics for antiviral applications. Conversely, the absence of these microbes in stool samples, as well as the presence of others found to associate with patients with poor recovery, as detected in NGS analysis of stool samples taken from individuals during routine biomedical tests and procedures, can form a diagnostic pattern of biomarkers that can predict the likelihood that said individuals are at risk for severe symptoms or poor recovery upon viral infection. This diagnostic may be based on the amount of one or more organisms present, position in the PCA plot, or other criteria that combines aspects of the whole genome sequence data. Reliability of such diagnostic is determined by the area under the ROC curve, as exemplified in FIG. 8. The diagnostic method can also detect gut microbial population patterns that can predict poor prognosis upon viral infection, thereby redirecting a patient to further diagnoses, appropriate life-style changes, or prophylactic treatments such as the administration of a live biotherapeutic or live biotherapeutics to restore healthy gut microbe populations.

In another embodiment, stool analysis is used as a diagnosis for viral infection. For example, specific DNA viruses of concern are detected by PCR or real time PCR (RT-PCR) using primers specific to the virus of concern. An analogous procedure is used for RNA viruses, with reverse transcription followed by RT-PCR. Alternatively, viruses can be detected non-specifically by whole genome sequencing. Total genomic DNA is extracted from the stool using the MagAttract PowerMicrobiome DNA/RNA EP kit (Qiagen), and from blood using the QIAamp DNA Blood Mini Kit (Qiagen). Genomic DNA is then prepared for Whole Genome Sequencing analysis using the sparQ DNA Frag & Library Prep kit (Quantabio). RNA is extracted from the stool or blood sample by binding to an RNeasy™ column (Qiagen) followed by washing and elution using the reagents provided in the RNeasy™ kit (Qiagen). Sequencing libraries are prepared from RNA by fragmentation, ribodepletion, cDNA synthesis, PCR amplification, and barcoding as described in the TRUSEQ® mRNA sample preparation kit (Illumina). Sequencing analysis is conducted on the Illumina platform using paired-end 150 bp reads. Reads not mapping to human or bacterial DNA are then aligned to a viral sequence database, for example the NCBI viral genomes database (https://www.ncbi.nlm.nih.gov/genome/viruses/). If a pathogenic virus is detected, remedial action can begin immediately.

### Example 23: Prophylactic Application of a Live Exemplary Biotherapeutic to Reduce Risk of Viral Infection or Improve Prognosis Upon Infection in Healthy Individuals

This example describes administration of a live exemplary biotherapeutic as provided herein, including a combination of bacteria as provided herein, for example, as set forth in Table 9, Example 10, and/or Table 42, Example 25, to an individual as a prophylactic in healthy individuals or individuals determined to be at risk of severe reaction to viral infection, such as those individuals that are immunocompromised, have a heart condition, or are over 70 years of age.

An individual is administered one of the present live biotherapeutics (Table 9, as described in Example 10, or genetically modified variants described in Examples 12 and 13, and/or as described in Table 42, Example 25), thereby conditioning the microbiome to best enable the individual's immune system to eliminate a virus rapidly upon infection. Specifically, the individual is administered a live biotherapeutic at a dose of between about 10⁵ to 10¹⁵ bacteria, or at a dose of about 10¹⁰, 10¹¹ or 10¹² bacteria total or per dose, which can be in a lyophilized form, for example, formulated in an enteric coated capsule. The individual takes 1, 2, 3, 4, 5, 6, 7, 8, 9, 10 or 11 or more live biotherapeutic capsules by mouth once, twice or three times per day, and resumes a normal diet after 2, 4, 8, 12, or 24 hours. In another embodiment, the individual may take the capsule by mouth before, during, or immediately after a meal.

### Example 24: Patient Data Collection from Clinical Trials and Machine Learning and Data Analysis on the Same

The results described here were obtained from a study involving cancer patients undergoing immunotherapy treatment and healthy controls. Microbes, gene functions, and metabolites elucidated as being absent in patients not responding well to treatment are relevant for the treatment of viral infections because in both cases a healthy immune response is required to combat the disease. Therefore, it is reasonable to expect that microbes beneficial for immuno-oncology treatment will also be beneficial or even essential for treating or ameliorating a viral infection, or for rapid viral clearance.

Eligible patients were selected based on current health condition, cancer status (current or in remission), and treatment program. Prior patient medical history was also collected and analyzed when available. This includes but is not limited to prior cancer history, diabetes, autoimmune disease, neurodegenerative disease, heart disease, metabolic syndrome, digestive disease, psychological disorders, HIV, and allergies. In addition, lifestyle and dietary habits were collected, including diet regimen, exercise routine, alcohol, nicotine, and caffeine intake, medical as well as recreational drug use, recent courses of antibiotics, vitamins, and probiotics. In some cases, information and data collected from wearable devices that monitor but is not limited to heart rate, calories burned, steps walked, blood pressure, biochemical release, time spent exercising and seizures. This data was assembled and used as input to the machine learning algorithms with the goal of determining correlations between patient history, wearable devices and treatment efficacy. In addition, relationships between this data and the results of sample analysis described below were elucidated.

In another embodiment, eligible patients testing positive for infection with COVID-19 (SARS-CoV2) or other coronavirus, or influenza virus, were selected as well as age-matched healthy controls. Information is also collected on the severity of disease, symptoms, time of recovery, and response to any treatment, if applicable. Prior patient medical history is also collected and analyzed, including but not limited to cancer, diabetes, autoimmune disease, neurodegenerative disease, heart disease, metabolic syndrome, digestive disease, psychological disorders, coronaviruses, influenza virus, HIV, and allergies. In addition, lifestyle and dietary habits are collected, including diet regimen, exercise routine, alcohol, nicotine, and caffeine intake, medical as well as recreational drug use, recent courses of antibiotics, vitamins, and probiotics. This data is assembled and used as input to the machine learning algorithms with the goal of determining correlations between patient history, course of illness, and results of stool and blood sample analysis

For current cancer patients, tumor size and cancer progression were tracked over time and classified based on radiographic assessment using the Response Criteria in Solid Tumors version 1.1 (Schwartz et al. Eur. J. Cancer 2016, 62:132-137) criteria. This is based on longitudinal measurements of lesions in cancer tissue, given a strict set of guidelines for lesion selection and measurement techniques. Responders to checkpoint inhibitor treatment are defined as patients that were cured or had stable disease lasting at least 6 months, while non-responders are defined as those whose cancer progressed or was stable for less than 6 months. Classification of responders and non-responders implies robust and insufficient immune response, respectively, and thus serves as a proxy for COVID-19, influenza, or other viral disease patients that will effectively clear the virus or have severe symptoms, respectively.

Each patient provided stool samples using the procedures as outlined in Example 2 and buccal swabs of the oral biome. In some cases, Urine, Blood and plasma samples were also taken by healthcare personnel within 1-2 days of the stool samples. Stool, urine and buccal samples were kept on ice or at 4° C. until processed. Whole blood was collected into an EDTA tube. Plasma was isolated from the blood by centrifugation at 1000×g for 10 minutes, followed by centrifugation at 2000×g for 10 minutes. At least three timepoints were taken for each patient, roughly every 6 to 8 weeks.

### Whole Genome Sequencing of Patient Fecal Samples

Whole genome sequencing was performed as previously described in Example 9 on a total of 450 fecal samples. Of the 450 samples, 322 samples were from cancer patients, 96 were from control subjects, and 32 were from subjects in remission. The results were classified, and abundance was estimated for each sample using centrifuge, using the publicly available GTDB database (Parks et al. (2019) bioRxiv 771964, Méric et al. (2019) bioRxiv 712166).

The results were analyzed for differential relative abundance of organisms between cancer and control cohorts, as well as correlations between relative abundance of organisms and immune markers, as measured by flow cytometry. Additionally, machine learning was performed to train a model capable of discriminating between a subject with cancer and a control subject.

Metagenomic sequences are also scanned to identify novel CRISPR sequences using a scoring algorithm such as that described in (Moreno-Mateos et al. (2015) Nat. Met. 12:982-988), and for predicted natural product gene clusters using the antiSMASH routine (Medema et al. (2011) Nuc. Acids Res. 39:W339-W346).

Table 33, illustrated as FIG. 47. Whole genome sequencing was performed on fecal samples from subjects with and without cancer and the reads were classified using the GTDB database and abundance of each species was estimated computationally (Centrifuge). For classified hits with a mean relative abundance of at least 0.005%, The fold change difference and statistical significance (inverse p value, Mann Whitney U test) was calculated for abundances between cancer and control sample cohorts.

### Cytokine Analysis of Blood Plasma

Plasma was obtained from 1 mL blood by centrifugation at 2000×g for 10 minutes. The plasma fraction was removed from the top and transferred to a clean tube. To remove any residual cells that may have carried over, the plasma was centrifuged again at 2000×g for 10 minutes, and the top layer was transferred to another tube, taking care to not take any red blood that may have settled to the bottom of the tube. Cytokine analysis was performed on 25 selected plasma samples by Eve Technologies (https://www.evetechnologies.com/) using the 48-plex Luminex assay.

Mann-Whitney test was applied to each cytokine to identify those with significant differential abundance between samples corresponding to checkpoint inhibitor complete responders (CR, N=6) and non-responders (NR, N=8). The remaining 11 samples were from patients identified as partial responders (PR) or stable disease (SD); due to the unclear phenotype, these were not included in the statistical analysis. Compounds with significant concentration differences between CR and NR samples (p<0.05) are listed in Table 34.

TABLE 34
Average fluorescence values for CR and NR samples
exhibiting significant differential abundance.

| | CR | NR | CR/NR | |
|---|---|---|---|---|
| Compound | Average | Average | ratio | P-value |
| Eotaxin | 20.91 | 36.11 | 0.58 | 0.0046 |
| IFNgamma | 0.51 | 4.41 | 0.12 | 0.0132 |
| IL.2 | 0.55 | 1.77 | 0.31 | 0.0141 |
| IL.27 | 1065.91 | 2326.60 | 0.46 | 0.0337 |
| MIP.1a | 30.02 | 44.87 | 0.67 | 0.0132 |

CyTOF Analysis of PBMCs Isolated from Whole Blood

Peripheral blood mononuclear cells (PBMCs) were isolated from approximately 8 mL blood using SEPMATE™ tubes following the manufacturer's instructions. Following isolation, cells were resuspended in 1 mL PBS+2% FBS. 10 uL of the cell suspension was mixed with 10 uL if Trypan Blue Stain 0.4% and applied to a cell counter plate to determine viable cell concentration. The cell suspension was then diluted in 90% PBS+10% DMSO to achieve a cell density of 1×10\hat{ }7 cells/mL. Cells were then frozen at a controlled rate of 1° C./min to a final temperature of −150° C. in liquid nitrogen.

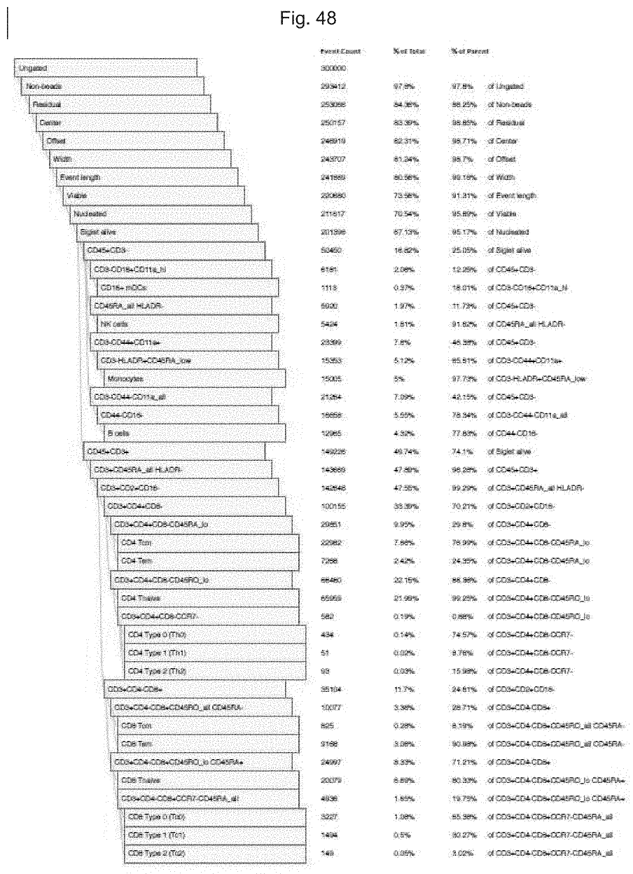

Mass cytometry (CyTOF) was performed on 25 selected PBMC samples by the University of Texas Health Center at San Antonio (UTHCSA). A 30 marker antibody panel focused on human immune-oncology relevant markers (Fluidigm) was used to quantify different cell populations. The markers and associated metal labels are given in Table 35. Markers were gated using the strategy shown in FIG. 48 to determine the immune cell types and subtypes. Cell populations were reported either as a percentage of all viable cells and/or of the parent cell type.

TABLE 35
List of antibodies and metal labels used for CyTOF analysis

| Immune Marker | Metal |
|---|---|
| CCR4 | 158Gd |
| CCR5 | 144Nd |
| CCR7 | 159Tb |
| CD11a | 142Nd |
| CD127 | 176Yb |
| CD134 [OX40] | 150Nd |
| CD137 [4-1BB] | 173Yb |
| CD152 [CTLA-4] | 161Dy |
| CD16 | 148Nd |
| CD161 | 164Dy |
| CD2 | 151Eu |
| CD223 [LAG3] | 175Lu |
| CD25 | 149Sm |
| CD27 | 167Er |
| CD278 [ICOS] | 168Er |
| CD279 [PD-1] | 155Gd |
| CD28 | 160Gd |
| CD3 | 170Er |
| CD366 [Tim-3] | 153Eu |
| CD4 | 145Nd |
| CD44 | 166Er |
| CD45 | 154Sm |
| CD45RA | 169Tm |
| CD45RO | 165Ho |
| CD49d | 141Pr |
| CD5 | 143Nd |
| CD57 | 172Yb |
| CD69 | 162Dy |
| CD7 | 147Sm |
| CD8a | 146Nd |
| CD9 | 171Yb |
| CD95 [Fas] | 152Sm |
| CXCR3 | 156Gd |
| HLA-DR | 174Yb |

Mann-Whitney test was applied to each population type or subtype to identify those with significant differential abundance between samples corresponding to checkpoint inhibitor complete responders (CR, N=6) and non-responders (NR, N=8). The remaining 11 samples were from patients identified as partial responders (PR) or stable disease (SD); due to the unclear phenotype, these were not included in the statistical analysis. Cell populations with significant abundance differences between CR and NR samples (p<0.05) are listed in Table 36.

TABLE 36
Cell population abundance values, as a percentage either of total
live cells or of the parent cell type, as indicated, for CR
and NR samples exhibiting significant
differential abundance.

| | CR | NR | CR/ | P- |
|---|---|---|---|---|
| Cell Population | Average | Average | NR | value |
| % of CD3−CD44+CD11a+ | 13.09 | 31.67 | 0.41 | 0.0132 |
| in Siglet alive | | | | |
| % of B cells in Siglet alive | 9.92 | 4.92 | 2.02 | 0.0046 |
| % of CD3−HLADR+CD45RA_ | 10.44 | 23.57 | 0.44 | 0.0095 |
| low in Siglet alive | | | | |
| % of Monocytes in Siglet alive | 10.12 | 22.29 | 0.45 | 0.0183 |
| % of CD3+CD4−CD8+CD45RO_ | 13.40 | 8.28 | 1.62 | 0.0337 |
| lo CD45RA+ in alive | | | | |
| % in B cells in CD45+CD3− | 43.37 | 26.18 | 1.66 | 0.0132 |

### Example 25: Data Driven and Machine Learning Approaches for Therapeutic Design

Whole genome sequencing and flow cytometry analysis were performed on human fecal and blood samples, respectively, as described in Example 24. A machine learning model was fit to discriminate cancer and control samples, using all fecal data collected to date. The model developed using the GTDB database was validated using Stratified Group K-Fold Cross Validation (Tables 37 to 38). In addition, linear discriminant analysis (LDA) effect size method (LEfSe) was used to classify microbes identified using the GTDB database enriched in cancer or control (Table 39).

TABLE 37
A logistic regression classifier was trained to classify
samples as corresponding to cancer or control on
samples with a mean relative abundance of at
least 0.005% using the GTDB database. An
ROC curve was generated on 322 cancer
samples and 92 control samples using
Stratified Group K-Fold Cross Validation
(AUC = 0.79). Following validation, the model
was trained on all the samples and
feature importance values are reported.

| Feature Importance | |
|---|---|
| (Logistic Regression) | Organism Name |
| 0.514417994 | Collinsella sp900548935 |
| 0.486287437 | Clostridium sp900539375 |
| 0.381613445 | UBA1191 sp900545775 |
| 0.310730798 | Raoultibacter massiliensis |
| 0.289945387 | Christensenella minuta |
| 0.283774901 | CAG-145 sp900540145 |
| 0.27456207 | Bacteroides stercoris |
| 0.26468198 | Erysipelatoclostridium sp900544435 |
| 0.263480075 | Phocaeicola salanitronis |
| 0.250041885 | Marvinbryantia sp900066075 |
| 0.249755758 | Odoribacter sp900544025 |
| 0.216103903 | UBA738 sp003522945 |
| 0.207027879 | An200 sp900550095 |
| 0.195934646 | Mediterraneibacter faecis |
| 0.185692545 | CAG-170 sp000436735 |
| 0.179847461 | Megasphaera elsdenii |
| 0.162281593 | Methanosphaera stadtmanae |
| 0.159663737 | UMGS1611 sp900553435 |
| 0.157611925 | CAG-177 sp003538135 |
| 0.157485555 | UBA6398 sp003150315 |
| 0.155329072 | CAG-492 sp000434015 |
| 0.153100473 | Dorea sp000433215 |
| 0.151760426 | Evtepia sp004556345 |
| 0.14588862 | UMGS1071 sp900542375 |
| 0.145040782 | Collinsella sp900554585 |
| 0.136236542 | Clostridium Q. sp003024715 |
| 0.131388743 | CAG-460 sp900544625 |
| 0.130605804 | Blautia Asp900551715 |
| 0.12874627 | Niameybacter sp900549765 |
| 0.127187848 | CAG-45 sp002299665 |
| 0.098447454 | Mailhella sp900541395 |
| 0.092072207 | SFFHOlsp900548125 |
| 0.080714744 | Dorea longicatena |
| 0.079070946 | Sutterella wadsworthensis_A |
| 0.076582096 | Negativibacillus sp000435195 |
| 0.073355953 | UMGS1590 sp900552455 |
| 0.061020643 | Coprococcus_A sp900548825 |
| 0.059560254 | Blautia_A sp900066335 |
| 0.058625801 | Eubacterium_ sp900557275 |
| 0.048160806 | Firm-11 sp900540045 |
| 0.0465729 | Dorea longicatena_B |
| 0.045683691 | UMGS1491 sp900554775 |
| 0.044846674 | UMGS1241 sp900549955 |
| 0.044173983 | CAG-1427 sp000436075 |
| 0.040847644 | Alistipes sp900541585 |
| 0.040245741 | Gemmiger variabilis |
| 0.039602886 | CAG-495 sp000432275 |
| 0.036058062 | Bariatricus comes |
| 0.035781984 | Oxalobacter formigenes |
| 0.03030392 | Frisingicoccus caecimuris |
| 0.025478979 | CAG-314 sp000437915 |
| 0.023104086 | QALW01 sp003150515 |
| 0.021151433 | Collinsella sp900554325 |
| 0.020407288 | CAG-485 sp900541835 |
| 0.020130762 | CAG-452 sp000434035 |
| 0.017010213 | Agathobacter sp900546625 |
| 0.016426446 | UBA5394sp003150565 |
| 0.005947673 | Blautia_A obeum_B |
| 0.004390397 | Coprobacillus cateniformis |
| 0.002233086 | Akkermansia sp004167605 |
| 0.00152013 | Anaerostipes hadrus_A |
| −0.001234426 | Limosilactobacillus fermentum_A |
| −0.003827343 | CAG-115sp003531585 |
| −0.008153089 | Fusobacterium_B sp900541465 |
| −0.014246241 | Prevotella sp900552515 |
| −0.016286555 | Collinsella sp900551665 |
| −0.021479219 | Anaerotignum lactatifermentans |
| −0.023122468 | UMGS1781 sp900553695 |
| −0.024041329 | Odoribacter laneus |
| −0.034455465 | UBA11471sp000434215 |
| −0.037849311 | Prevotellamassilia sp000437675 |
| −0.039128417 | Angelakisella sp900547385 |
| −0.039646845 | Agathobaculum sp900291975 |
| −0.041056608 | Eubacterium_R sp000434995 |
| −0.04266878 | Eubacterium_F sp900539115 |
| −0.044059805 | Alistipes sp000434235 |
| −0.050522202 | UMGS1590 sp900553245 |
| −0.051836169 | UMGS1688 sp900554085 |
| −0.057847833 | Butyricimonas faecalis |
| −0.066253286 | Akkermansia muciniphila_A |
| −0.067189759 | Coprobacter fastidiosus |
| −0.067646141 | CAG-83 sp900550585 |
| −0.083533993 | Prevotella sp900554045 |
| −0.085318406 | Intestinimonas butyriciproducens |
| −0.093860595 | Eubacterium_F sp000434115 |
| −0.103834319 | Eubacterium_R sp900540305 |
| −0.106144597 | Desulfovibrio fairfieldensis |
| −0.113985815 | Lachnospira sp900316325 |
| −0.117390396 | Porphyromonas sp000768875 |
| −0.122672447 | Acidaminococcus intestini |
| −0.126358887 | CAG-303 sp000437755 |
| −0.127237507 | Bacteroides caccae |
| −0.136509832 | Prevotella sp900548745 |
| −0.136915786 | Dorea sp000433535 |
| −0.137055372 | Ligilactobacillus salivarius |
| −0.151411951 | Blautia_A sp900551465 |
| −0.174551647 | CAG-83 sp000431575 |
| −0.182703866 | Streptococcus vestibularis |
| −0.188088114 | CAG-302 sp900543825 |
| −0.191528797 | Butyricimonas virosa |
| −0.207519696 | Dialister sp900343095 |
| −0.208796646 | Streptococcus sp000314795 |
| −0.21979495 | QANA01 sp900554725 |
| −0.220254926 | Enterococcus_B faecium |
| −0.249373565 | COE1 sp001916965 |
| −0.249871731 | Mailhella sp003150275 |
| −0.251086664 | Lachnospira eligens |
| −0.299023203 | Catenibacterium sp000437715 |
| −0.303053041 | GCA-900066755 sp900066755 |
| −0.30357643 | CAG-1031sp000431215 |
| −0.306860922 | UBA1691sp900544375 |
| −0.318039896 | CAG-495 sp001917125 |
| −0.32832744 | AM07-15 sp003477405 |
| −0.387480395 | Ruthenibacterium sp003149955 |
| −0.441806113 | Parabacteroides johnsonii |
| −0.513157387 | Bariatricus massiliensis |

TABLE 38
A logistic regression classifier was trained to
classify samples as corresponding to cancer
(non-responder) or control on samples with a mean
relative abundance of at least 0.005% using the
GTDB database. An ROC curve was generated
on 43 non-responder samples and 92 control
samples using Stratified Group K-Fold Cross
Validation (AUC = 0.71). Following validation,
the model was trained on all the samples
and feature importance values are reported.

| Feature Importance | |
|---|---|
| (Logistic Regression) | Organism Name |
| 0.568597444 | CAG-170 sp000436735 |
| 0.543705645 | Coprobacillus cateniformis |
| 0.509426281 | Mailhella sp900541395 |
| 0.482820632 | Blautia_A sp003474435 |
| 0.471483202 | UMGS1611 sp900553435 |
| 0.244782119 | UMGS911sp900557415 |
| 0.184527891 | CAG-354 sp900553015 |
| 0.174892874 | Blautia_A massiliensis |
| 0.158545809 | Agathobacter sp900317585 |
| 0.140717769 | Negativibacillus sp000435195 |
| 0.127151205 | Prevotella sp002251385 |
| 0.122995374 | Coprococcus_A sp900548825 |
| 0.118746116 | Alistipes_A indistinctus |
| 0.118323236 | UMGS1071 sp900542375 |
| 0.115663494 | Erysipelatoclostridium sp900544435 |
| 0.10338765 | Collinsella sp900547285 |
| 0.102410987 | Prevotella sp900556825 |
| 0.094993875 | UMGS172 sp900539855 |
| 0.06348916 | Phocaeicola sp900551445 |
| 0.061539232 | Agathobacter rectalis |
| 0.056113717 | Anaerobutyricum hallii |
| 0.053598211 | Blautia_A sp900066335 |
| 0.053249701 | Anaerostipes hadrus_A |
| 0.045497159 | Clostridium sp001916075 |
| 0.037406556 | Holdemanella sp003458715 |
| 0.021590668 | Christensenella minuta |
| 0.002218293 | Collinsella sp900541725 |
| 4.2957E−05 | Phascolarctobacterium faecium |
| −0.004703489 | Bacteroides togonis |
| −0.008809374 | Paraprevotella clara |
| −0.03119867 | Holdemania sp900120005 |
| −0.031492474 | AM51-8 sp900546435 |
| −0.035434119 | Phil1 sp001940855 |
| −0.038913964 | Schaedlerella sp004556565 |
| −0.044020829 | Lachnospira sp900552795 |
| −0.047515072 | Muricomes sp900604355 |
| −0.052967481 | Prevotella buccae |
| −0.071596115 | Longicatena sp003433845 |
| −0.0796651 | Desulfovibrio fairfieldensis |
| −0.100975915 | Lachnospira sp003537285 |
| −0.115966192 | Butyricimonas faecihominis |
| −0.172472377 | Blautia_A sp900551465 |
| −0.187868969 | Anaerotruncus massiliensis |
| −0.19109635 | Anaerofustis stercorihominis |
| −0.206509093 | UMGS1688 sp900544575 |
| −0.210914586 | Bifidobacterium dentium |
| −0.228226067 | Bacteroides cutis |
| −0.241407669 | F23-B02 sp001916715 |
| −0.247678711 | COE1 sp001916965 |
| −0.267182222 | Ruminococcus_E bromii_B |
| −0.286160011 | Porphyromonas sp001552775 |
| −0.323514014 | UBA1691sp900544715 |
| −0.335225188 | GCA-900066755 sp900066755 |
| −0.340598662 | Eubacterium_G sp900548465 |
| −0.35989301 | Limosilactobacillus fermentum_A |
| −0.460367032 | Mesosutterella massiliensis |
| −0.475293296 | Escherichia flexneri |
| −0.542914883 | Enterococcus_B faecium |
| −0.599141069 | CAG-521sp000437635 |
| −0.675358406 | Phocaeicola sp000436795 |
| −0.774574761 | CAG-83 sp900550585 |

TABLE 39
Linear discriminant analysis (LDA) effect size method (LEfSe) was used to
classify microbes (GTDB database) enriched in cancer or control. Analysis was
conducted on 322 cancer samples and 96 control samples. LEfSe first identifies
features that are statistically different among various populations using the non-
parametric factorial Kruskal-Wallis (KW) sum-rank test; It then performs additional
pairwise tests to assess whether these differences are consistent with respect to
population subclasses using the unpaired Wilcoxon rank-sum test. Lastly, LEfSe uses
LDA to estimate the effect size of each differentially abundant feature. A total of 135
species were enriched in cancer patients and 189 species were enriched in healthy individuals.

| | | | LDA | |
|---|---|---|---|---|
| | | Enrichment | score | p-value |
| taxID | Organism Name | Group | (log10) | (Kruskal-Wallis test) |
| 17568 | Blautia_A | Cancer | 2.13760 | 0.0010911377336 |
| | sp900120195 | | | |
| 17532 | Blautia coccoides | Cancer | 2.39200 | 0.000968237855956 |
| 17534 | Blautia hansenii | Cancer | 2.61227 | 0.0216950428348 |
| 17535 | Blautia hominis | Cancer | 2.00955 | 0.0132436138036 |
| 17536 | Blautia sp000432195 | Cancer | 2.64642 | 6.48111804063e−05 |
| 38844 | Streptococcus mutans | Cancer | 2.19809 | 0.000766810025194 |
| 21762 | Eisenbergiella tayi | Cancer | 2.09239 | 0.0257014664011 |
| 18508 | CAG-273 sp000437855 | Cancer | 2.19293 | 0.00149432368338 |
| 22144 | Escherichia | Cancer | 2.24534 | 0.000447045218029 |
| | sp000208585 | | | |
| 20468 | Coprococcus eutactus | Cancer | 2.35758 | 0.00917122961097 |
| 17540 | Blautia sp003287895 | Cancer | 2.62746 | 1.36005693245e−05 |
| 17547 | Blautia sp900556555 | Cancer | 2.05367 | 0.0264114231619 |
| 17148 | Bacteroides | Cancer | 2.10570 | 0.00282381576978 |
| | bouchesdurhonensis | | | |
| 15906 | Anaerostipes | Cancer | 2.01922 | 0.00114807012388 |
| | sp000508985 | | | |
| 14115 | 43-108 sp001915545 | Cancer | 2.64298 | 1.11878531695e−06 |
| 36509 | Ruminococcus_H | Cancer | 2.33650 | 0.00895450765663 |
| | sp900549945 | | | |
| 15832 | Anaerobutyricum | Cancer | 2.23321 | 0.0225920389673 |
| | hallii_A | | | |
| 36428 | Ruminococcus_A | Cancer | 2.53016 | 0.00139750682718 |
| | sp000432335 | | | |
| 25300 | Hungatella | Cancer | 2.20734 | 2.41051912452e−07 |
| | sp005845265 | | | |
| 31012 | Oscillibacter welbionis | Cancer | 2.97773 | 0.000181598180603 |
| 23244 | Fusobacterium_B | Cancer | 2.05886 | 0.00968800303448 |
| | sp900541465 | | | |
| 21884 | Enterocloster | Cancer | 2.53945 | 6.46879345659e−10 |
| | aldenensis | | | |
| 26966 | Longicatena innocuum | Cancer | 2.62342 | 0.000826173225832 |
| 38939 | Streptococcus | Cancer | 2.54944 | 0.000251671138383 |
| | sp000187445 | | | |
| 20690 | Cronobacter sakazakii | Cancer | 2.00001 | 0.00176581732956 |
| 20055 | Clostridium_Q | Cancer | 2.60684 | 4.89158492686e−09 |
| | symbiosum | | | |
| 15178 | Agathobacter | Cancer | 2.00539 | 0.0481413837296 |
| | sp000434275 | | | |
| 21731 | Eggerthella lenta | Cancer | 2.94523 | 0.006101347123 |
| 38891 | Streptococcus | Cancer | 2.42213 | 2.6511957776e−05 |
| | parasanguinis_D | | | |
| 38889 | Streptococcus | Cancer | 2.56043 | 0.000319467593934 |
| | parasanguinis_B | | | |
| 38888 | Streptococcus | Cancer | 2.33233 | 9.51509478653e−05 |
| | parasanguinis_A | | | |
| 38887 | Streptococcus | Cancer | 2.35117 | 0.00071108799438 |
| | parasanguinis | | | |
| 22512 | Faecalimonas | Cancer | 2.23748 | 0.012095387025 |
| | sp900556835 | | | |
| 19869 | Citrobacter freundii | Cancer | 2.07185 | 0.00906080909617 |
| 23068 | Flavonifractor | Cancer | 2.96730 | 2.14986779138e−09 |
| | sp000508885 | | | |
| 33819 | Providencia rettgeri_D | Cancer | 2.20376 | 0.00955245042142 |
| 17543 | Blautia sp900541955 | Cancer | 2.50204 | 0.0173404800143 |
| 32690 | Phocaeicola dorei | Cancer | 3.66861 | 0.000216406212684 |
| 32695 | Phocaeicola plebeius | Cancer | 2.44370 | 0.00392932331679 |
| 32699 | Phocaeicola sartorii | Cancer | 2.18940 | 0.00108006311504 |
| 18772 | CAG-83 sp001916855 | Cancer | 2.01482 | 0.00380931699685 |
| 17198 | Bacteroides | Cancer | 2.48723 | 0.0172953376659 |
| | sp900557355 | | | |
| 17196 | Bacteroides | Cancer | 2.44481 | 0.00508686372198 |
| | sp900556215 | | | |
| 17191 | Bacteroides | Cancer | 2.18952 | 4.52735545739e−05 |
| | sp900066265 | | | |
| 44733 | Veillonella atypica | Cancer | 2.18847 | 0.0402708440438 |
| 27993 | Mediterraneibacter | Cancer | 3.55674 | 0.0496897855081 |
| | torques | | | |
| 38890 | Streptococcus | Cancer | 2.20714 | 0.000599054128583 |
| | parasanguinis_C | | | |
| 21757 | Eisenbergiella | Cancer | 2.18135 | 0.00915828126624 |
| | sp900539715 | | | |
| 17157 | Bacteroides faecis | Cancer | 2.61898 | 0.000484436396227 |
| 15918 | Anaerotruncus | Cancer | 2.22604 | 0.00133142885356 |
| | colihominis | | | |
| 38951 | Streptococcus | Cancer | 2.92682 | 0.00217861964326 |
| | sp001556435 | | | |
| 18579 | CAG-45 sp900066395 | Cancer | 2.49950 | 0.00574455333834 |
| 17554 | Blautia_A | Cancer | 3.19213 | 2.23528859735e−06 |
| | sp000433815 | | | |
| 21497 | Dorea scindens | Cancer | 2.79558 | 2.09572874651e−05 |
| 26866 | Limosilactobacillus | Cancer | 2.34117 | 0.0103155939811 |
| | fermentum | | | |
| 17205 | Bacteroides | Cancer | 3.21745 | 0.00012372735459 |
| | xylanisolvens | | | |
| 21888 | Enterocloster | Cancer | 3.08276 | 3.13138339085e−12 |
| | clostridioformis | | | |
| 21886 | Enterocloster bolteae | Cancer | 2.81644 | 2.15012098387e−ll |
| 18199 | Butyricimonas | Cancer | 2.14057 | 4.33439876776e−05 |
| | faecihominis | | | |
| 41906 | UBA1691 | Cancer | 3.44725 | 2.33522633211e−10 |
| | sp900544375 | | | |
| 25980 | Klebsiella variicola | Cancer | 2.09542 | 0.0155919157839 |
| 21889 | Enterocloster | Cancer | 2.54158 | 2.41667650124e−07 |
| | clostridioformis_A | | | |
| 36521 | Ruthenibacterium | Cancer | 2.77271 | 8.80361828788e−05 |
| | lactatiformans | | | |
| 26241 | Lachnospira | Cancer | 2.04108 | 0.0461403001471 |
| | sp000436535 | | | |
| 15835 | Anaerobutyricum | Cancer | 2.11887 | 0.0181255372277 |
| | sp900016875 | | | |
| 21501 | Dorea sp000433535 | Cancer | 3.18022 | 8.50501585649e−07 |
| 15033 | Acutalibacter | Cancer | 2.20583 | 2.07942112135e−05 |
| | sp900543555 | | | |
| 17156 | Bacteroides | Cancer | 2.00310 | 0.00578699520994 |
| | faecichinchillae | | | |
| 17150 | Bacteroides | Cancer | 2.30080 | 1.18027718738e−06 |
| | caecimuris | | | |
| 44095 | UBA9502 | Cancer | 2.56934 | 0.000419156172297 |
| | sp900538475 | | | |
| 32688 | Phocaeicola coprocola | Cancer | 3.05308 | 0.0168486380976 |
| 39618 | Succiniclasticum | Cancer | 2.14647 | 0.0262474166286 |
| | sp900544275 | | | |
| 17197 | Bacteroides | Cancer | 2.67332 | 0.0181020544302 |
| | sp900556625 | | | |
| 18198 | Butyricimonas faecalis | Cancer | 2.54767 | 2.02297766283e−06 |
| 36434 | Ruminococcus_B | Cancer | 3.40611 | 0.00343207293924 |
| | gnavus | | | |
| 36436 | Ruminococcus_C | Cancer | 2.59013 | 3.11571237196e−06 |
| | callidus | | | |
| 37769 | Sellimonas intestinalis | Cancer | 2.90188 | 0.0010421172501 |
| 14650 | Acidaminococcus | Cancer | 2.84483 | 5.37086074804e−06 |
| | intestini | | | |
| 38929 | Streptococcus | Cancer | 2.93889 | 0.0133708816272 |
| | salivarius | | | |
| 31909 | Parabacteroides | Cancer | 3.49346 | 0.00845055659135 |
| | distasonis | | | |
| 26428 | Lawsonibacter | Cancer | 2.27921 | 0.00119563602136 |
| | sp900066825 | | | |
| 15902 | Anaerostipes caccae | Cancer | 2.59029 | 1.32175359294e−05 |
| 22142 | Escherichia flexneri | Cancer | 3.07841 | 0.000448573849446 |
| 39003 | Streptococcus | Cancer | 2.91268 | 1.23303428895e−06 |
| | vestibularis | | | |
| 17204 | Bacteroides uniformis | Cancer | 3.76744 | 0.00767040878452 |
| 22082 | Erysipelatoclostridium | Cancer | 3.10882 | 4.49001877438e−05 |
| | ramosum | | | |
| 17179 | Bacteroides rodentium | Cancer | 2.36982 | 0.000809140186422 |
| 25979 | Klebsiella | Cancer | 2.50337 | 0.0209044295048 |
| | quasivariicola | | | |
| 38737 | Streptococcus | Cancer | 2.56735 | 0.0450278679091 |
| | anginosus_C | | | |
| 19879 | Citrobacter youngae | Cancer | 2.10125 | 0.0268286601359 |
| 32689 | Phocaeicola | Cancer | 2.39012 | 8.788249368e−06 |
| | coprophilus | | | |
| 33237 | Prevotella | Cancer | 2.05312 | 0.00100376799716 |
| | sp000257925 | | | |
| 23067 | Flavonifractor plautii | Cancer | 2.95190 | 2.03840497779e−09 |
| 22140 | Escherichia | Cancer | 2.72638 | 0.000178346031215 |
| | dysenteriae | | | |
| 21898 | Enterocloster | Cancer | 2.07701 | 0.0133349033913 |
| | sp900541315 | | | |
| 21890 | Enterocloster | Cancer | 2.05837 | 5.58379720897e−08 |
| | lavalensis | | | |
| 17201 | Bacteroides | Cancer | 3.50101 | 0.0235802219915 |
| | thetaiotaomicron | | | |
| 38946 | Streptococcus | Cancer | 2.35186 | 3.7304387141e−05 |
| | sp000448565 | | | |
| 26964 | Longicatena | Cancer | 2.71146 | 0.000140435878 |
| | caecimuris | | | |
| 31921 | Parabacteroides | Cancer | 2.04037 | 0.000627513743016 |
| | sp900155425 | | | |
| 21401 | Dialister sp900343095 | Cancer | 2.42134 | 0.0326132648947 |
| 18336 | CAG-103 sp900543625 | Cancer | 2.23674 | 0.000902041176381 |
| 17180 | Bacteroides salyersiae | Cancer | 2.66579 | 2.13189055395e−05 |
| 18337 | CAG-1031 | Cancer | 2.57987 | 0.000146355127079 |
| | sp000431215 | | | |
| 21512 | Dorea sp900543415 | Cancer | 2.70565 | 3.39380545885e−10 |
| 32682 | Phill2 sp002633275 | Cancer | 2.31286 | 0.00199992224058 |
| 22509 | Faecalimonas | Cancer | 2.27491 | 0.0151214093109 |
| | sp900550975 | | | |
| 22186 | Eubacterium_G | Cancer | 2.14863 | 0.000467276332765 |
| | ventriosum | | | |
| 22513 | Faecalimonas | Cancer | 2.77402 | 0.0436444293568 |
| | umbilicata | | | |
| 32208 | Parasutterella | Cancer | 2.31846 | 0.0356205438601 |
| | sp000980495 | | | |
| 32727 | Phocaeicola vulgatus | Cancer | 3.87915 | 0.0142224019801 |
| 18334 | CAG-103 sp900317855 | Cancer | 2.18662 | 0.0252397542571 |
| 26805 | Ligilactobacillus | Cancer | 2.50862 | 0.000565702472295 |
| | salivarius | | | |
| 17147 | Bacteroides | Cancer | 2.02458 | 0.000193059075288 |
| | acidifaciens | | | |
| 33256 | Prevotella | Cancer | 2.00053 | 0.0228785615692 |
| | sp001275135 | | | |
| 32637 | Phascolarctobacterium | Cancer | 3.07562 | 0.00859527040104 |
| | faecium | | | |
| 19917 | Clostridioides difficile | Cancer | 2.14826 | 0.000396856053418 |
| 17574 | Blautia_A | Cancer | 2.00426 | 0.00105097878926 |
| | sp900547615 | | | |
| 18469 | CAG-217 sp900547275 | Cancer | 2.17756 | 0.0150594036026 |
| 18461 | CAG-194 sp000432915 | Cancer | 2.41211 | 0.0114380924628 |
| 17578 | Blautia_A | Cancer | 2.09782 | 8.7672215879e−05 |
| | sp900551465 | | | |
| 31913 | Parabacteroides | Cancer | 2.32757 | 7.57370993477e−07 |
| | johnsonii | | | |
| 36435 | Ruminococcus_B | Cancer | 2.32731 | 0.000138031227462 |
| | sp900544395 | | | |
| 17188 | Bacteroides | Cancer | 2.09073 | 0.00116767135794 |
| | sp003545565 | | | |
| 18649 | CAG-492 sp000434335 | Cancer | 2.02629 | 0.000100614508133 |
| 41907 | UBA1691 | Cancer | 2.86541 | 7.87326411749e−07 |
| | sp900544715 | | | |
| 17160 | Bacteroides fragilis | Cancer | 2.62172 | 0.0152421440534 |
| 31910 | Parabacteroides | Cancer | 2.20437 | 0.0138650485607 |
| | distasonis_A | | | |
| 17186 | Bacteroides | Cancer | 2.21447 | 0.00117561051251 |
| | sp002491635 | | | |
| 17189 | Bacteroides | Cancer | 2.61541 | 4.67903060213e−07 |
| | sp003865075 | | | |
| 20471 | Coprococcus | Cancer | 2.05457 | 8.85142109464e−08 |
| | sp000433075 | | | |
| 17167 | Bacteroides | Cancer | 2.99981 | 0.0304061339071 |
| | intestinalis | | | |
| 17168 | Bacteroides | Cancer | 2.49123 | 0.011609802117 |
| | intestinalis_A | | | |
| 21894 | Enterocloster | Cancer | 2.40734 | 0.00141864996401 |
| | sp001517625 | | | |
| 17154 | Bacteroides cutis | Cancer | 2.12726 | 0.038819131362 |
| 36679 | SFFH01 sp900542445 | Control | 2.41059 | 3.30021813163e−06 |
| 36440 | Ruminococcus_C | Control | 3.20407 | 1.2103932064e−08 |
| | sp000980705 | | | |
| 41347 | UBA11524 | Control | 2.03222 | 0.00829874351407 |
| | sp000437595 | | | |
| 20338 | Collinsella | Control | 2.08313 | 1.11217975236e−08 |
| | sp900556415 | | | |
| 22089 | Erysipelatoclostridium | Control | 2.42399 | 1.1132866617e−06 |
| | sp900544435 | | | |
| 22087 | Erysipelatoclostridium | Control | 2.25783 | 2.64720533759e−10 |
| | sp003024675 | | | |
| 20324 | Collinsella | Control | 2.21598 | 1.04125747668e−08 |
| | sp900554905 | | | |
| 22085 | Erysipelatoclostridium | Control | 2.96769 | 4.21528595137e−ll |
| | sp000752095 | | | |
| 20321 | Collinsella | Control | 2.00425 | 0.000781585095622 |
| | sp900554645 | | | |
| 36447 | Ruminococcus_D | Control | 3.39071 | 6.09724921548e−06 |
| | bicirculans | | | |
| 18401 | CAG-1427 | Control | 2.05861 | 0.000239442448034 |
| | sp000435675 | | | |
| 15198 | Agathobaculum | Control | 2.45929 | 0.00687534588514 |
| | sp900625105 | | | |
| 17538 | Blautia sp001304935 | Control | 2.76252 | 0.00021477069236 |
| 44369 | UMGS1241 | Control | 2.48651 | 0.000229158432302 |
| | sp900549955 | | | |
| 26970 | Longicatena | Control | 2.04471 | 0.0253206102387 |
| | sp900411325 | | | |
| 15193 | Agathobaculum | Control | 2.72460 | 2.55177393509e−06 |
| | sp003481705 | | | |
| 22497 | Faecalibacterium | Control | 2.45021 | 0.00114034752317 |
| | sp900539885 | | | |
| 15191 | Agathobaculum | Control | 2.40050 | 9.30926345453e−05 |
| | butyriciproducens | | | |
| 18588 | CAG-460 sp900544625 | Control | 2.45197 | 0.00302957091049 |
| 36473 | Ruminococcus_E | Control | 2.36465 | 0.0116416270466 |
| | sp003438075 | | | |
| 25246 | Holdemanella | Control | 2.39373 | 5.39071908999e−06 |
| | sp900551285 | | | |
| 25245 | Holdemanella | Control | 2.13257 | 0.010514300225 |
| | sp900547815 | | | |
| 25244 | Holdemanella | Control | 2.16423 | 0.000129153396687 |
| | sp003458715 | | | |
| 18402 | CAG-1427 | Control | 2.02925 | 0.00989909203149 |
| | sp000436075 | | | |
| 20131 | Collinsella | Control | 2.50700 | 9.18497857285e−06 |
| | aerofaciens_G | | | |
| 22491 | Faecalibacterium | Control | 2.61316 | 1.05261675827e−06 |
| | prausnitzii_J | | | |
| 17200 | Bacteroides stercoris | Control | 3.05136 | 0.042749136853 |
| 18416 | CAG-1427 | Control | 2.27099 | 0.0267898981999 |
| | sp900556585 | | | |
| 41454 | UBA1191 | Control | 2.21447 | 9.43642971559e−06 |
| | sp900545775 | | | |
| 22490 | Faecalibacterium | Control | 2.65573 | 1.19439741311e−05 |
| | prausnitzii_l | | | |
| 20339 | Collinsella | Control | 2.31396 | 1.12520743428e−05 |
| | sp900556445 | | | |
| 23770 | GCA-900066135 | Control | 2.13602 | 2.91176542674e−08 |
| | sp900543575 | | | |

| | | | | |
|---|---|---|---|---|
| 17575 | Blautia_A | Control | 2.61892 | 1.33647845585e−07 |
| | sp900548245 | | | |
| 18784 | CAG-83 sp900547745 | Control | 2.05677 | 0.000783754622785 |
| 17344 | Bifidobacterium | Control | 3.89216 | 0.0472240909999 |
| | adolescentis | | | |
| 25848 | KLE1615 sp900066985 | Control | 2.60806 | 1.08359063226e−05 |
| 17562 | Blautia_A | Control | 2.06312 | 0.000228185017914 |
| | sp900066145 | | | |
| 32723 | Phocaeicola | Control | 2.61412 | 0.032610595599 |
| | sp900553715 | | | |
| 41455 | UBA1191 | Control | 2.50874 | 8.12599169877e−05 |
| | sp900549125 | | | |
| 21661 | ER4 sp000765235 | Control | 2.34673 | 0.000662818789913 |
| 18331 | CAG-103 sp000432375 | Control | 2.87783 | 5.58314397621e−07 |
| 37771 | Sellimonas | Control | 2.51538 | 0.0307760188802 |
| | sp002161525 | | | |
| 18338 | CAG-110 sp000434635 | Control | 2.74437 | 6.44929544018e−05 |
| 24117 | Gemmiger | Control | 2.05341 | 4.66989314069e−05 |
| | sp900539695 | | | |
| 24112 | Gemmiger formicilis | Control | 2.44504 | 0.00333770203037 |
| 33197 | Prevotella copri_A | Control | 2.65431 | 0.0283791459871 |
| 24118 | Gemmiger | Control | 2.16137 | 1.2620604518e−05 |
| | sp900540595 | | | |
| 44359 | UMGS1071 | Control | 2.09882 | 0.000974900001284 |
| | sp900542375 | | | |
| 21409 | Dialister sp900555245 | Control | 2.63436 | 0.00333770203037 |
| 22173 | Eubacterium_F | Control | 2.36858 | 3.51840365919e−05 |
| | sp003491505 | | | |
| 21500 | Dorea sp000433215 | Control | 2.33715 | 1.97114808776e−08 |
| 19946 | Clostridium saudiense | Control | 2.17730 | 0.0230705246422 |
| 19949 | Clostridium | Control | 2.05791 | 0.00616715858917 |
| | sp000435835 | | | |
| 17560 | Blautia_A | Control | 2.33260 | 2.52783279408e−06 |
| | sp003478765 | | | |
| 17563 | Blautia_A | Control | 2.88048 | 0.00298338599418 |
| | sp900066165 | | | |
| 17566 | Blautia_A | Control | 2.55805 | 5.85243145116e−06 |
| | sp900066355 | | | |
| 41419 | UBA11774 | Control | 2.48337 | 0.0450169284476 |
| | sp003507655 | | | |
| 17559 | Blautia_A | Control | 2.26051 | 0.00340034813393 |
| | sp003477525 | | | |
| 21493 | Dorea longicatena | Control | 3.31604 | 5.28875486139e−09 |
| 17565 | Blautia_A | Control | 2.75595 | 9.24085724755e−11 |
| | sp900066335 | | | |
| 18785 | CAG-83 sp900548615 | Control | 2.00739 | 0.00287952903975 |
| 18783 | CAG-83 sp900545585 | Control | 2.51557 | 4.66505048711e−06 |
| 17413 | Bifidobacterium | Control | 2.50849 | 0.00126433385994 |
| | sp002742445 | | | |

| | | | | |
|---|---|---|---|---|
| 15188 | Agathobacter | Control | 2.36342 | 0.000411600171088 |
| | sp900550845 | | | |
| 15183 | Agathobacter | Control | 2.45178 | 0.00017678701388 |
| | sp900546625 | | | |
| 15181 | Agathobacter | Control | 2.82123 | 0.000142395876762 |
| | sp900317585 | | | |
| 15186 | Agathobacter | Control | 2.34044 | 0.0348666029549 |
| | sp900549895 | | | |
| 18651 | CAG-492 sp900553225 | Control | 2.53389 | 0.000269274517967 |
| 19908 | Cloacibacillus | Control | 2.01540 | 0.0327228894077 |
| | porcorum | | | |
| 18241 | Butyrivibrio_A | Control | 2.37277 | 0.00126177720823 |
| | crossotus | | | |
| 18243 | Butyrivibrio_A | Control | 2.29993 | 0.000757328088867 |
| | sp900543865 | | | |
| 20287 | Collinsella | Control | 2.10823 | 5.77069184548e−06 |
| | sp900551365 | | | |
| 44382 | UMGS1375 | Control | 2.31480 | 0.00204908177496 |
| | sp900066615 | | | |
| 14550 | Acetatifactor | Control | 2.28979 | 0.00350713933387 |
| | sp900066365 | | | |
| 17230 | Barnesiella | Control | 2.45687 | 0.00109479017124 |
| | intestinihominis | | | |
| 17558 | Blautia_A | Control | 2.11266 | 1.13585564617e−09 |
| | sp003474435 | | | |
| 27982 | Mediterraneibacter | Control | 3.12051 | 3.79965372336e−09 |
| | faecis | | | |
| 44383 | UMGS1375 | Control | 2.10198 | 1.99431838413e−06 |
| | sp900551235 | | | |
| 17549 | Blautia_Amassiliensis | Control | 3.35483 | 9.0678522457e−06 |
| 44304 | UCG-010 sp003150115 | Control | 2.07626 | 1.93454349489e−08 |
| 40350 | Terrisporobacter | Control | 2.27319 | 0.0201956139629 |
| | sp900557165 | | | |
| 17555 | Blautia_A | Control | 2.77455 | 1.50755393035e−07 |
| | sp000436615 | | | |
| 17550 | Blautia_A obeum | Control | 3.28966 | 5.87804863778e−05 |
| 17551 | Blautia_A obeum_B | Control | 2.10105 | 0.00117560721128 |
| 18491 | CAG-269 sp003525075 | Control | 2.94816 | 1.10382253131e−06 |
| 30848 | Odoribacter laneus | Control | 2.42804 | 0.02830176635 |
| 17564 | Blautia_A | Control | 2.57852 | 3.49087257679e−ll |
| | sp900066205 | | | |
| 15043 | Adlercreutzia | Control | 2.07439 | 0.0164550857066 |
| | celatus_A | | | |
| 36088 | Roseburia | Control | 2.43035 | 0.0265096583225 |
| | inulinivorans | | | |
| 20185 | Collinsella | Control | 2.45893 | 1.78086972764e−07 |
| | sp900541475 | | | |
| 22483 | Faecalibacterium | Control | 2.53717 | 1.48409829345e−07 |
| | prausnitzii_A | | | |
| 14374 | AM51-8 sp003478275 | Control | 2.04747 | 0.001001970988 |
| 21494 | Dorea longicatena_B | Control | 3.01526 | 4.84949138423e−07 |
| 18771 | CAG-83 sp000435975 | Control | 2.58227 | 0.00441888019065 |
| 18673 | CAG-533 sp000434495 | Control | 2.22485 | 0.00514615742321 |
| 18475 | CAG-245 sp000435175 | Control | 2.22165 | 0.0435372051499 |
| 15831 | Anaerobutyricum hallii | Control | 3.09528 | 0.00014746116909 |
| 15836 | Anaerobutyricum | Control | 2.70371 | 0.000916439523968 |
| | sp900554965 | | | |
| 22482 | Faecalibacterium | Control | 3.19093 | 2.18287972068e−06 |
| | prausnitzii | | | |
| 20276 | Collinsella | Control | 2.08412 | 7.91309114891e−05 |
| | sp900550185 | | | |
| 20272 | Collinsella | Control | 2.49443 | 1.41645935111e−08 |
| | sp900549455 | | | |
| 24122 | Gemmiger | Control | 2.36155 | 4.05221257543e−06 |
| | sp900554145 | | | |
| 44754 | Veillonella | Control | 2.24720 | 0.0330231957551 |
| | sp900556785 | | | |
| 36477 | Ruminococcus_E | Control | 3.37778 | 0.0144137210572 |
| | sp003526955 | | | |
| 21892 | Enterocloster | Control | 2.38993 | 0.00857107906373 |
| | sp000431375 | | | |
| 18445 | CAG-177 sp003538135 | Control | 2.07697 | 0.000716445637701 |
| 40005 | TF01-11 sp001414325 | Control | 2.66655 | 0.000363496777214 |
| 17366 | Bifidobacterium | Control | 2.20765 | 0.0114038100379 |
| | catenulatum | | | |
| 26235 | Lachnospira eligens_B | Control | 2.56502 | 0.0154865827583 |
| 36087 | Roseburia intestinalis | Control | 3.09407 | 0.0279857822864 |
| 23215 | Fusicatenibacter | Control | 3.44159 | 2.62500713715e−06 |
| | saccharivorans | | | |
| 19959 | Clostridium | Control | 2.64288 | 0.000134946507533 |
| | sp900540255 | | | |
| 30930 | Olsenella_E | Control | 2.10495 | 9.32929729361e−05 |
| | sp003150175 | | | |
| 18510 | CAG-273 sp003507395 | Control | 3.10648 | 7.55800913874e−06 |
| 36438 | Ruminococcus_C | Control | 2.50387 | 0.00215829381827 |
| | sp000437175 | | | |
| 17579 | Blautia_A | Control | 2.07825 | 5.77555977528e−12 |
| | sp900551715 | | | |
| 18631 | CAG-485sp900541835 | Control | 2.21771 | 0.00687270499319 |
| 20052 | Clostridium_Q | Control | 2.13420 | 5.46070638709e−05 |
| | sp003024715 | | | |
| 18846 | CAG-964 sp000435335 | Control | 2.16784 | 0.0427477385672 |
| 21907 | Enterococcus faecalis | Control | 2.55268 | 0.000973238270192 |
| 17233 | Barnesiella | Control | 2.22322 | 0.00884314924177 |
| | sp003150885 | | | |
| 17404 | Bifidobacterium | Control | 2.66841 | 0.00855402261808 |
| | ruminantium | | | |
| 29933 | Negativibacillus | Control | 2.17199 | 0.0422015141754 |
| | sp000435195 | | | |
| 18346 | CAG-110 sp003525905 | Control | 2.33171 | 0.00175739825879 |
| 20478 | Coprococcus_A | Control | 2.31292 | 1.03016482881e−08 |
| | sp900548825 | | | |
| 40012 | TF01-11 sp003529475 | Control | 2.55554 | 2.41121115527e−08 |
| 18426 | CAG-170 sp000432135 | Control | 2.41207 | 0.000193772855899 |
| 22237 | Eubacterium_R | Control | 2.42086 | 0.00209914829707 |
| | sp000433975 | | | |
| 22498 | Faecalibacterium | Control | 2.93189 | 8.29721553513e−07 |
| | sp900539945 | | | |
| 22499 | Faecalibacterium | Control | 2.40470 | 0.000272278325887 |
| | sp900540455 | | | |
| 22484 | Faecalibacterium | Control | 3.14427 | 3.49956264954e−08 |
| | prausnitzii_C | | | |
| 44737 | Veillonella dispar_A | Control | 2.58590 | 0.011756073445 |
| 22199 | Eubacterium_I | Control | 2.44993 | 0.000781473771325 |
| | ramulus | | | |
| 20133 | Collinsella | Control | 2.42591 | 4.45271353249e−07 |
| | aerofaciens_I | | | |
| 23216 | Fusicatenibacter | Control | 2.62476 | 0.00684254308783 |
| | sp900543115 | | | |
| 18577 | CAG-45 sp000438375 | Control | 2.02767 | 0.0318370090133 |
| 36437 | Ruminococcus_C | Control | 2.14431 | 8.48212740565e−05 |
| | sp000433635 | | | |
| 30995 | Oscillibacter | Control | 2.10314 | 0.000287891879703 |
| | sp001916835 | | | |
| 18843 | CAG-95 sp900066375 | Control | 2.50108 | 0.00140687944173 |
| 18482 | CAG-269 sp000437215 | Control | 2.72572 | 0.028711054205 |
| 15903 | Anaerostipes hadrus | Control | 3.45161 | 4.07263251646e−05 |
| 44517 | UMGS743 | Control | 2.14080 | 0.00458547437347 |
| | sp900545085 | | | |
| 36674 | SFEL01 sp004557245 | Control | 2.08035 | 1.95437989749e−05 |
| 15904 | Anaerostipes | Control | 3.04152 | 7.21486613455e−09 |
| | hadrus_A | | | |
| 44405 | UMGS1491 | Control | 2.24642 | 0.000330918704025 |
| | sp900554775 | | | |
| 36508 | Ruminococcus_H | Control | 2.95780 | 0.000800746402152 |
| | sp003531055 | | | |
| 15468 | Alistipes sp000434235 | Control | 2.30490 | 0.0124196560779 |
| 18511 | CAG-273 sp003534295 | Control | 2.70758 | 0.0159319228906 |
| 20477 | Coprococcus_Acatus | Control | 2.29379 | 1.01475697641e−06 |
| 20167 | Collinsella | Control | 2.39447 | 9.35633056563e−09 |
| | sp900540895 | | | |
| 36429 | Ruminococcus_A | Control | 2.42518 | 7.23070236119e−05 |
| | sp000437095 | | | |
| 20473 | Coprococcus | Control | 2.25740 | 1.14391166168e−08 |
| | sp900066115 | | | |
| 25571 | Intestinibacter | Control | 2.27861 | 0.0497710656561 |
| | sp900540355 | | | |
| 17226 | Bariatricus comes | Control | 3.17691 | 4.0099294784e−10 |
| 36096 | Roseburia | Control | 2.24323 | 0.0231106169811 |
| | sp900552665 | | | |
| 24113 | Gemmiger qucibialis | Control | 3.16098 | 2.99842836046e−05 |
| 22496 | Faecalibacterium | Control | 2.23613 | 1.90744603181e−07 |
| | sp003449675 | | | |

| | | | | |
|---|---|---|---|---|
| 43535 | UBA7182 | Control | 2.09576 | 1.84717329358e−07 |
| | sp003481535 | | | |
| 24119 | Gemmiger | Control | 2.56966 | 1.80894410074e−07 |
| | sp900540775 | | | |
| 36431 | Ruminococcus_A | Control | 2.66427 | 5.49451308118e−08 |
| | sp003011855 | | | |
| 18480 | CAG-269 sp000431335 | Control | 3.04994 | 6.96985807634e−06 |
| 18484 | CAG-269 sp001915995 | Control | 2.01969 | 0.025830997993 |
| 18485 | CAG-269 sp001916005 | Control | 2.10988 | 3.25915762025e−07 |
| 18648 | CAG-492 sp000434015 | Control | 2.21000 | 1.30539446398e−07 |
| 26240 | Lachnospira | Control | 2.55618 | 0.000628363206244 |
| | sp000436475 | | | |
| 18679 | CAG-536 sp000434355 | Control | 2.82212 | 0.00456621092046 |
| 15176 | Agathobacter rectalis | Control | 3.41921 | 0.000310178035846 |
| 21491 | Dorea formicigenerans | Control | 2.80870 | 5.93297276419e−06 |
| 26245 | Lachnospira | Control | 2.56136 | 0.0023267608224 |
| | sp003451515 | | | |
| 18509 | CAG-273 sp000438355 | Control | 2.76462 | 0.0305438756183 |
| 20345 | Collinsella | Control | 2.00151 | 2.5548792017e−07 |
| | sp900557455 | | | |
| 20342 | Collinsella | Control | 3.12583 | 9.53488707721e−05 |
| | sp900556605 | | | |
| 25241 | Holdemanella biformis | Control | 2.52660 | 0.00128748752692 |
| 36078 | Romboutsia | Control | 2.47361 | 0.000416035368085 |
| | timonensis | | | |
| 18438 | CAG-170 sp900556635 | Control | 2.15669 | 2.45053105973e−05 |
| 28004 | Megasphaera | Control | 2.70507 | 0.0488599018113 |
| | sp000417505 | | | |
| 22488 | Faecalibacterium | Control | 2.98235 | 1.81752622445e−05 |
| | prausnitzii_G | | | |
| 22489 | Faecalibacterium | Control | 2.77389 | 3.98763445095e−06 |
| | prausnitzii_H | | | |
| 18433 | CAG-170 sp900545925 | Control | 2.09412 | 1.07072435143e−06 |
| 20469 | Coprococcus | Control | 2.96335 | 0.00490028391811 |
| | eutactus_A | | | |
| 19952 | Clostridium | Control | 2.47249 | 9.76605249819e−06 |
| | sp001916075 | | | |
| 33438 | Prevotella | Control | 2.63503 | 0.0105872123676 |
| | sp900551275 | | | |
| 27983 | Mediterraneibacter | Control | 2.82901 | 3.28992544736e−05 |
| | lactaris | | | |
| 17358 | Bifidobacterium | Control | 2.90693 | 0.00395513293096 |
| | bifidum | | | |
| 22277 | Evtepia sp004556345 | Control | 2.09867 | 0.000584164019016 |
| 40011 | TF01-11 sp003524945 | Control | 2.99299 | 0.0106460099673 |
| 22486 | Faecalibacterium | Control | 2.31452 | 6.15296343593e−06 |
| | prausnitzii_E | | | |
| 26247 | Lachnospira | Control | 2.63101 | 0.0141817065492 |
| | sp900316325 | | | |

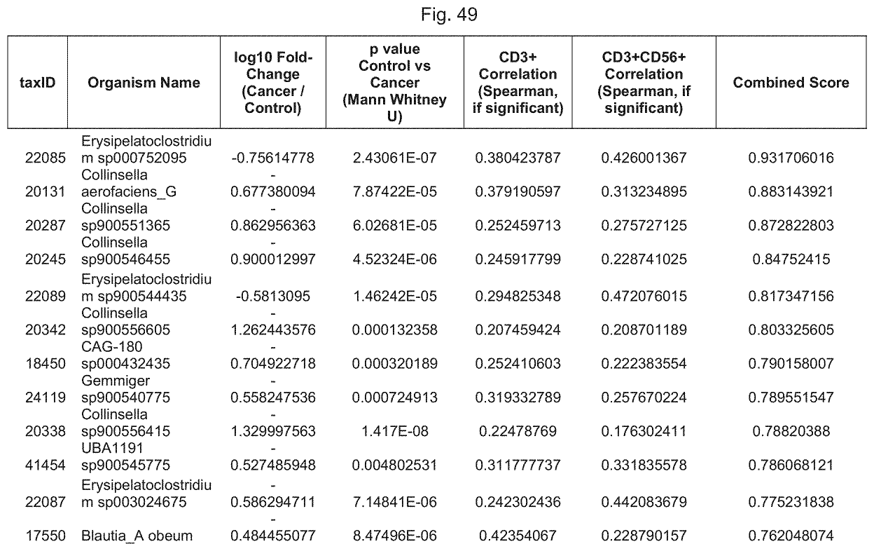

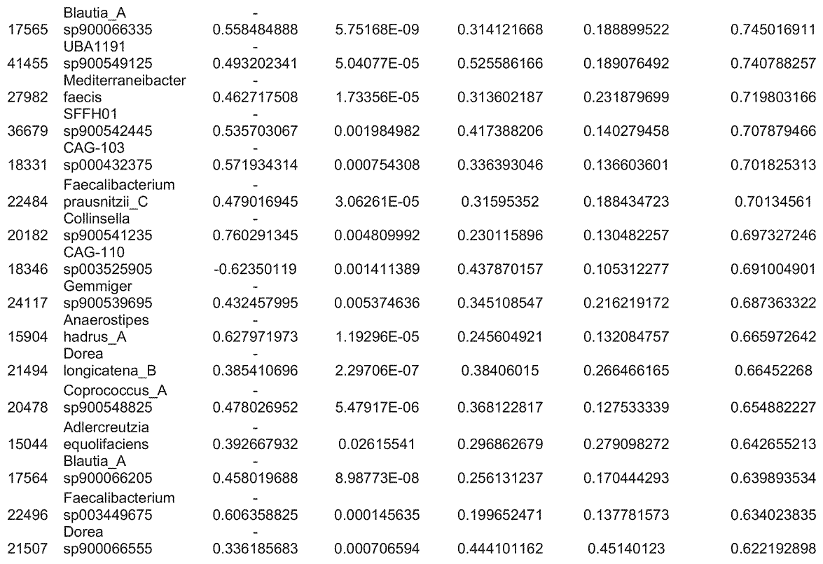

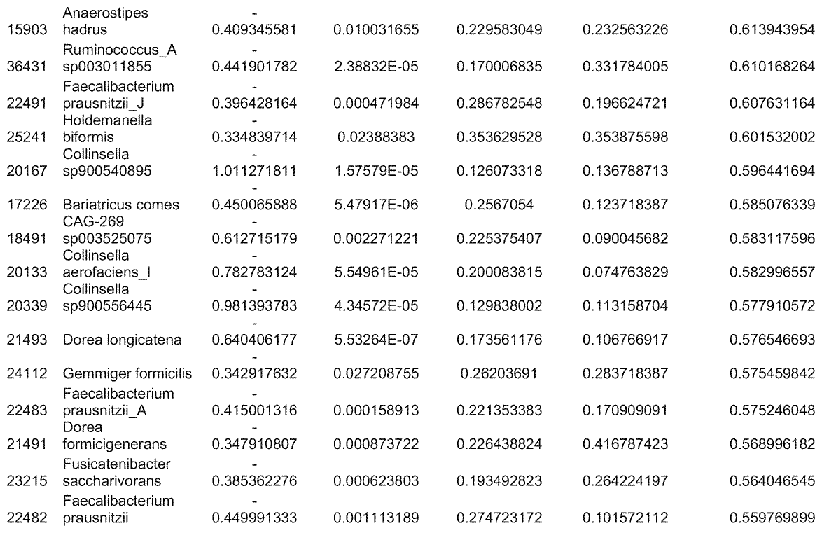

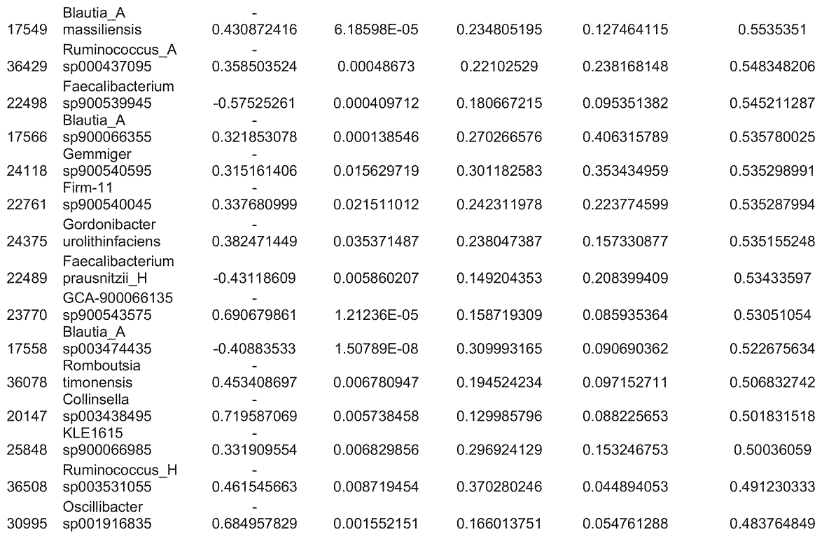

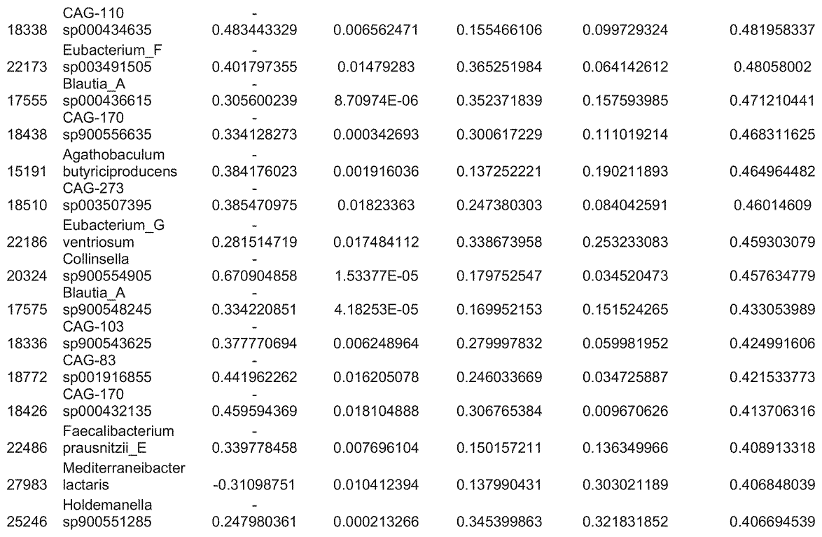

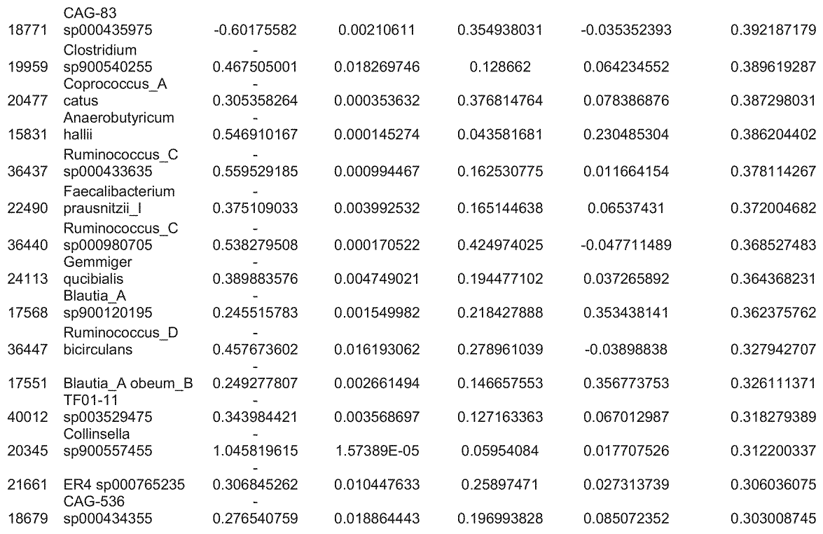

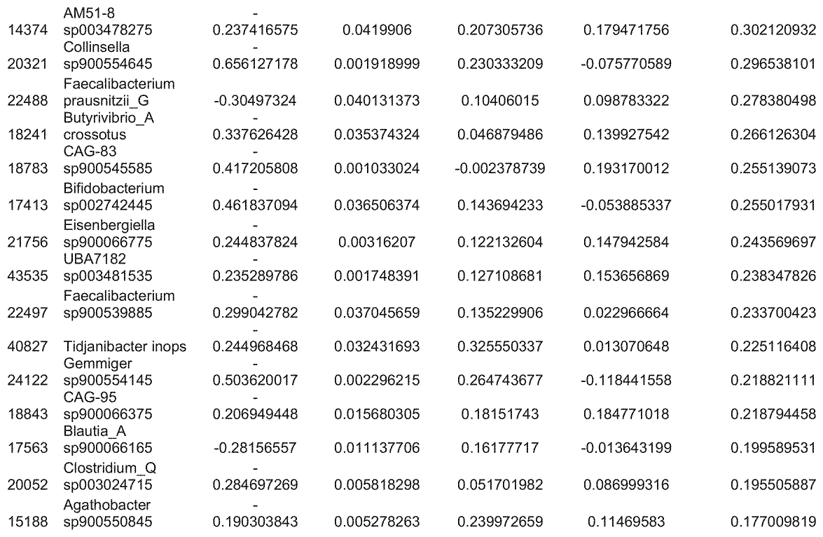

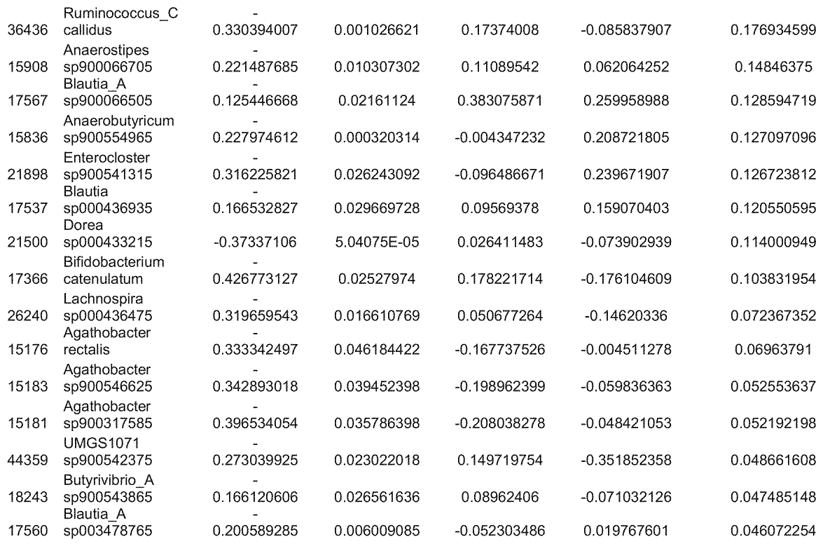

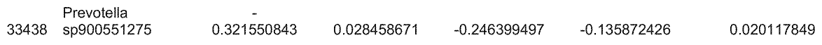

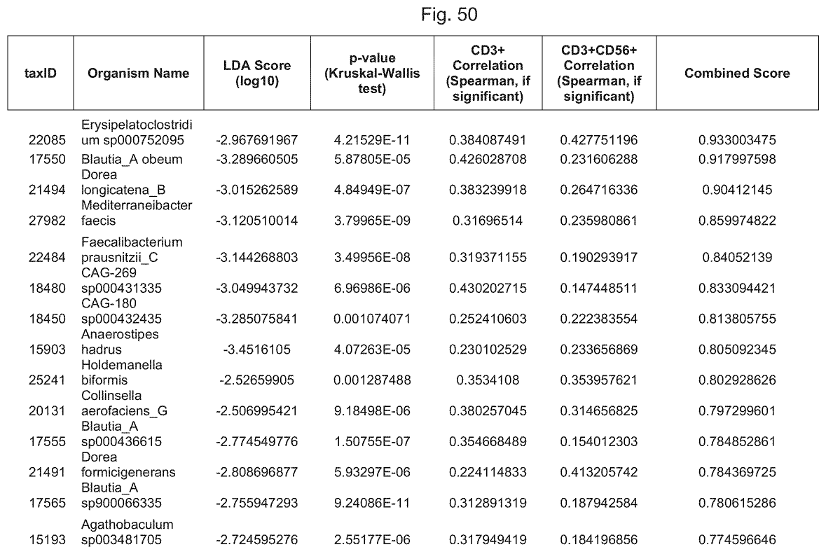

A composite score was then assigned to each organism, accounting for both their correlations to immune markers and fold change between cancer and control cohorts (Tables 40, and 41). The score is defined as the geometric mean of three metrics: fold change between cancer and control samples, CD3+ correlation, and CD3+CD56+ correlation.
Table 40 (illustrated as FIG. 49). Microbe rankings were based on classified species results using the GTDB database with a mean abundance of at least 0.005% with significant differences between cancer and control cohorts for inclusion into the therapeutic (inverse p value, Mann Whitney U test). For each classified species hit, CD3+ and CD3+CD56+ correlations are included in the table as per the linear mixed model analysis or set to zero if the mixed model correlation is negative or if the Spearman correlation was not significant enough to necessitate mixed model analysis. The cancer and control fold change, CD3+ correlation, and CD3+CD56+ correlation for each OSU were converted to percentile scores, and a combined score for each species level hit was generated by computing the geometric mean of each of the three percentiles.
Table 41 (illustrated as FIG. 50). Microbe rankings were based on classified species results using the GTDB database with a mean abundance of at least 0.005% with significant differences between cancer and control cohorts for inclusion into the therapeutic (LDA score, LEfSe). For each classified species hit, CD3+ and CD3+CD56+ correlations are included in the table as per the linear mixed model analysis or set to zero if the mixed model correlation is negative or if the Spearman correlation was not significant enough to necessitate mixed model analysis. The cancer and control fold change, CD3+ correlation, and CD3+CD56+ correlation for each OSU were converted to percentile scores, and a combined score for each species level hit was generated by computing the geometric mean of each of the three percentiles.

### Machine Learning for Live Biotherapeutic Design

The top 6 scoring organisms from Table 40 is selected for screening in simulated microbial mixes. Each combination of 4 organisms from the top 6 (listed in Table 42, below) is evaluated in silico using the trained machine learning model. For the cancer samples in the model, relative species abundances for the four organisms in the putative mix are increased in silico by a certain amount (here 0.5%). This simulates in silico the physical action of adding microbes to the gut microbiome. Classification is then performed using the machine learning model to estimate the probability that each augmented sample is a cancer sample. The hypothesis is that combinations of microbes that make cancer samples appear more like control samples according to the model are better candidates for therapeutic mixes. Each putative mix is scored by its mean predicted cancer probability across all the augmented cancer samples, with lower mean predicted cancer probabilities corresponding to notionally better therapeutic candidates. All of the exemplary live biotherapeutic compositions (exemplary microbial combinations) are then validated experimentally as described in Examples 18 and 19.

TABLE 42
The top 6 scoring organisms using LEfSe from Table
40 have been selected for screening in simulated
microbial mixes. All possible combinations of 4
organisms from the top 6 are shown.

| | Organism Name |
|---|---|
| Mix 1 | Erysipelatoclostridium sp000752095 |
| | Blautia_A obeum |
| | Mediterraneibacter faecis |
| | Faecalibacterium prausnitzii_C |
| Mix 2 | Blautia_A obeum |
| | Dorea longicatena_B |
| | Mediterraneibacter faecis |
| | Faecalibacterium prausnitzii_C |
| Mix 3 | Blautia_A obeum |
| | Dorea longicatena_B |
| | Mediterraneibacter faecis |
| | CAG-269 sp000431335 |
| Mix 4 | Erysipelatoclostridium sp000752095 |
| | Blautia_A obeum |
| | Dorea longicatena_B |
| | Faecalibacterium prausnitzii_C |
| Mix 5 | Erysipelatoclostridium sp000752095 |
| | Dorea longicatena_B |
| | Faecalibacterium prausnitzii_C |
| | CAG-269 sp000431335 |
| Mix 6 | Erysipelatoclostridium sp000752095 |
| | Blautia_A obeum |
| | Dorea longicatena_B |
| | CAG-269 sp000431335 |
| Mix 7 | Erysipelatoclostridium sp000752095 |
| | Blautia_A obeum |
| | Faecalibacterium prausnitzii_C |
| | CAG-269sp000431335 |
| Mix 8 | Erysipelatoclostridium sp000752095 |
| | Dorea longicatena_B |
| | Mediterraneibacter faecis |
| | Faecalibacterium prausnitzii_C |
| Mix 9 | Dorea longicatena_B |
| | Mediterraneibacter faecis |
| | Faecalibacterium prausnitzii_C |
| | CAG-269 sp000431335 |
| Mix 10 | Erysipelatoclostridium sp000752095 |
| | Mediterraneibacter faecis |
| | Faecalibacterium prausnitzii_C |
| | CAG-269 sp000431335 |
| Mix 11 | Blautia_A obeum |
| | Dorea longicatena_B |
| | Faecalibacterium prausnitzii_C |
| | CAG-269 sp000431335 |
| Mix 12 | Blautia_A obeum |
| | Mediterraneibacter faecis |
| | Faecalibacterium prausnitzii_C |
| | CAG-269 sp000431335 |
| Mix 13 | Erysipelatoclostridium sp000752095 |
| | Dorea longicatena_B |
| | Mediterraneibacter faecis |
| | CAG-269 sp000431335 |
| Mix 14 | Erysipelatoclostridium sp000752095 |
| | Blautia_A obeum |
| | Mediterraneibacter faecis |
| | CAG-269 sp000431335 |
| Mix 15 | Erysipelatoclostridium sp000752095 |
| | Blautia_A obeum |
| | Dorea longicatena_B |
| | Mediterraneibacter faecis |

### Example 26—Therapeutic Effect of Fecal Microbiota Transplant (FMT) on Influenza Infection with Antibiotic Pretreatment

In previous experiments performed by Persephone Biosciences and other researchers, Fecal Material Transplant (FMT) from responder and non-responder patients to mice with ectopicly introduced tumors and treated with anti-PD-1 immunotherapy agents showed that the mice receiving responder FMT showed greater immunotherapy-induced tumor shrinkage than those receiving FMT from non-responder patients. As there could be immunological similarities between the responses against tumors and viral infection, the immunological effects of healthy control and non-responsive cancer patient FMTs on mice challenged with lethal doses of influenza A/California/04/2009 (H1N1) virus was investigated. Mortality, weight loss, mean day of death, lung virus titers, lung pathology scores and weights and lung cytokine concentrations were the primary endpoints.

Female 6-week-old BALB/c mice were obtained from Charles River Laboratories (Wilmington, Mass.) for this experiment. The mice were quarantined for 3 days before use and maintained on Teklad Rodent Diet (Harlan Teklad) and tap water at the Laboratory Animal Research Center of Utah State University (USU). A total of 82 mice were randomized into 4 groups of 18 mice per group with 10 mice used as normal controls for weight gain (Table 43). During week 1 of the study, mice were treated with antibiotic mix aliquots by oral (PO) administration of 0.1 ml daily for five days. The antibiotic mix aliquots contained 1 mg/mL each of ampicillin, gentamicin, metronidazole, neomycin, and 0.5 mg/mL vancomycin.

TABLE 43
Experimental design for viral infection challenge experiment.

| | Number | | | |
|---|---|---|---|---|
| | Mice per | FMT | | |
| Group | Group | Treatment | Virus | Observations |
| 1 | 18 | Healthy | A/CA/04/2009 | 9 animals observed for weight loss and |
| | | Control | (H1N1pdm) | mortality for 14 days post-virus exposure. |
| 2 | 18 | Non | | 9 animals per group sacrificed on day 3 |
| | | Responder | | post-infection for lung virus titers, lung |
| | | | | weights, lung scores, and lung cytokine concentrations. |
| 3 | 18 | Healthy | No Virus | 9 animals observed for weight loss and |
| | | Control | | mortality for 14 days post-virus exposure. |
| 4 | 18 | Non | | 9 animals per group sacrificed on day 3 |
| | | Responder | | post-infection for lung virus titers, lung |
| | | | | weights, lung scores, and lung cytokine concentrations. |
| 5 | 10 | No FMT | No Virus | Normal controls for weight gain. |

On Week 2 of the study, mice were then given FMT treatments every day for five days by PO administration of a 0.2 ml volume of FMT preparation. Two fecal sample sets, each representing a single fecal donation from two test subjects, were used to create FMT preparations for the study. One of these (PBT-138) was donated by a subject at late stage non-small cell lung cancer who had failed to respond to Keytruda as Non-Responder, or NR). The other sample set, (PBT-208) was donated by a subject in good health and without history of cancer or cancer-related disease or complications (hereto referred as Healthy Control, or HC). Two groups of 18 mice each were provided the NR-FMT, while two groups of 18 mice each were provided the HC-FMT. Upon receipt, fecal material was homogenized with anoxic phosphate-buffered saline (PBS) containing 1 g/L cysteine and 30% glycerol in an anaerobic chamber with an atmosphere of 5% hydrogen, 10% carbon dioxide, and 85% nitrogen, then frozen in 1.2 mL aliquots and stored at −80° C. Prior to the experiment, FMT aliquots were thawed in the anaerobic chamber, combined, and diluted 10-fold in PBS containing 0.5 g/L cysteine and 15% glycerol. The solution was stirred on ice for 10 minutes until homogenous, then poured through 100 micron mesh filters to remove gross particulates. Aliquots of 15 ml were pipetted into fresh appropriately labeled 50 ml conical tubes and kept upright on ice. The headspace atmosphere of each tube was then exchanged for 100% argon by introducing a stream of filtered argon into the top of the tube for 10 seconds, after which the tube was tightly recapped. These single-use aliquots were stored at −80° C., and removed immediately prior to administering to the mice. FMT treatments were continued twice weekly for weeks 3, 4, and 5 of the study.

At the beginning of week 6 of the study, 2 treatment groups of mice, one representing HC-FMT and the other representing NR-FMT, were challenged with mouse-adapted influenza A/California/04/2009 (H1N1) (A/CA/04/2009; H1N1pdm) via intranasal route. The virus was received from Dr. Elena Govorkova, Department of Infectious Diseases, St. Jude Children's Research Hospital, Memphis Tenn. The virus was adapted to replication in the lungs of BALB/c mice by 9 sequential passages in mice. Virus was plaque purified in Madin-Darby Canine Kidney (MDCK) cells and a virus stock was prepared by growth in embryonated chicken eggs and then MDCK cells. For challenge, mice were anesthetized by intraperitoneal (IP) injection of ketamine/xylazine (50 mg/kg/5 mg/kg) prior to challenge by the intranasal route with approximately 1×103 (3×LD50) cell culture infectious doses (CCID50) of virus per mouse in a 90 μl inoculum volume. Nine mice per treatment group were euthanized 72 hours after influenza infection for evaluation of lung virus titers and cytokine concentrations. The remaining nine mice per group were weighed prior to influenza challenge then every day thereafter to assess the effects of FMT on preventing weight loss due to virus infection. All mice were observed for morbidity and mortality through day 14 post-influenza challenge.

### Post-Infection Effects on Survival and Body Weight

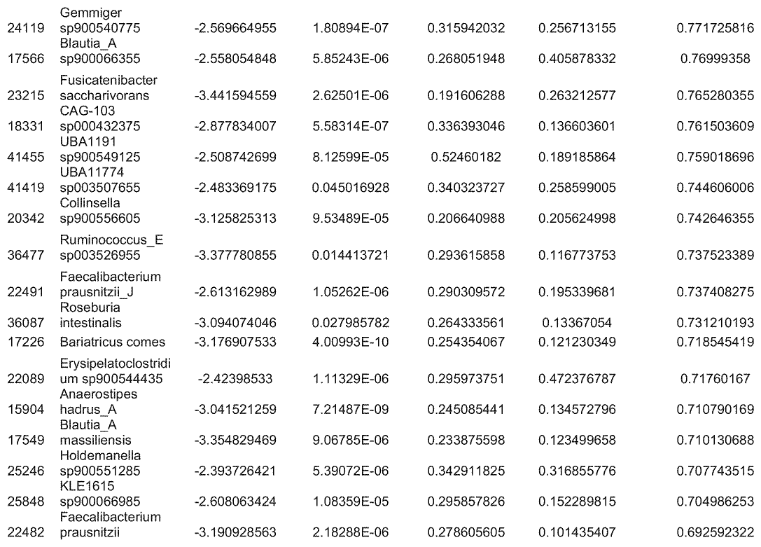

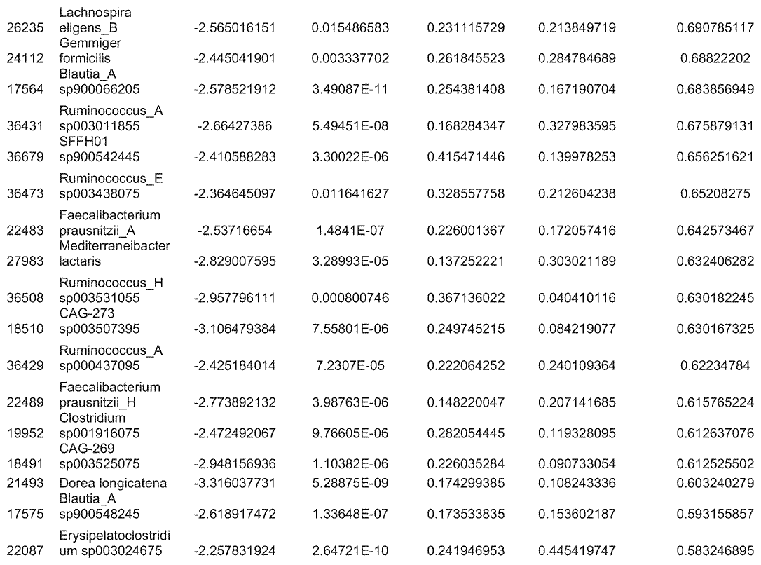

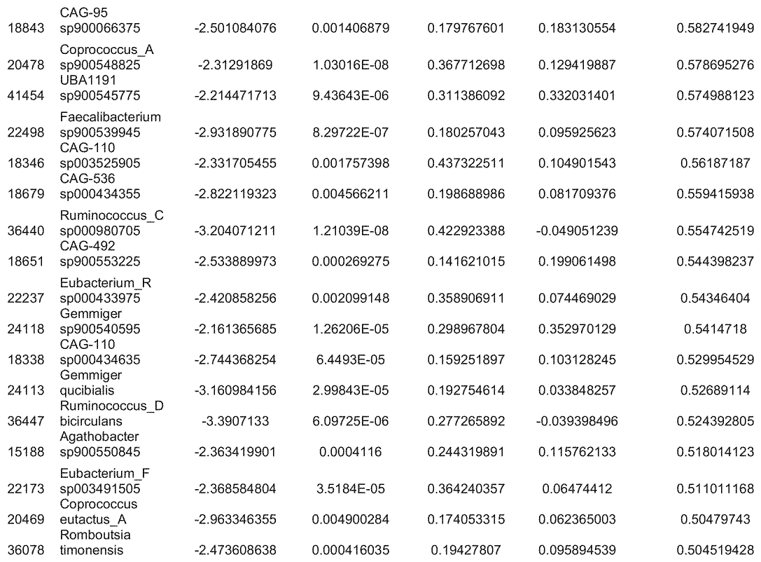

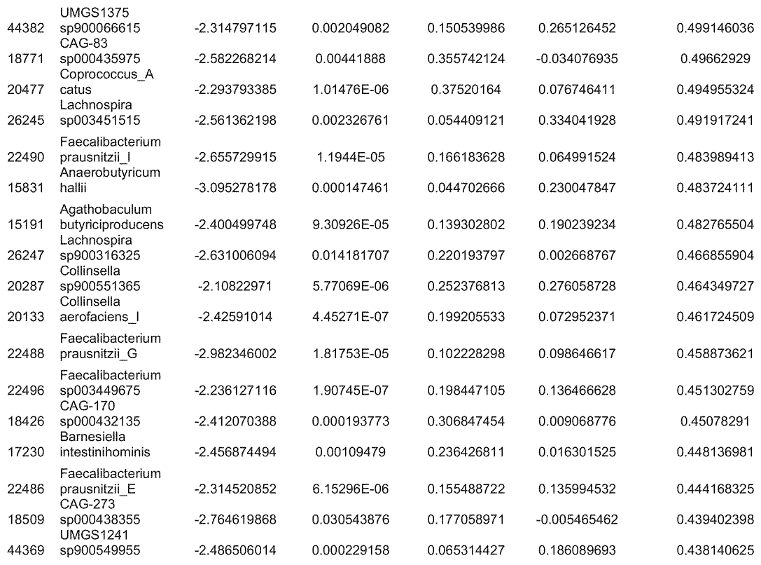

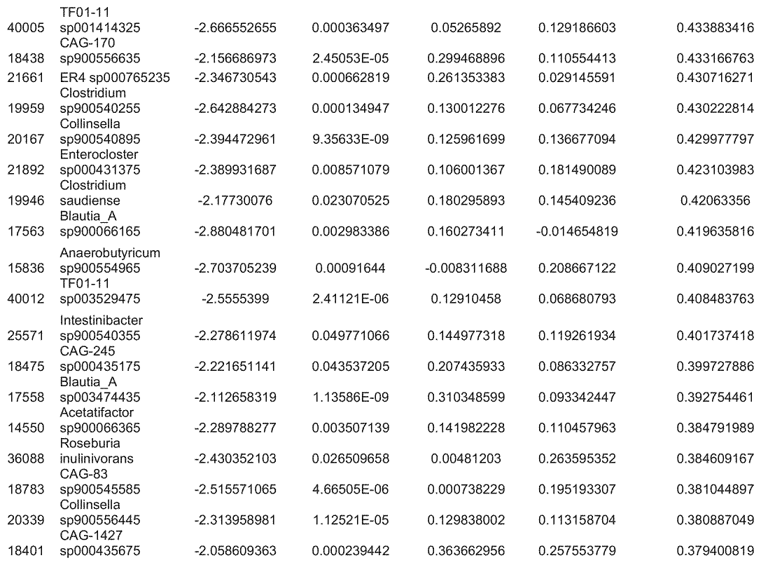

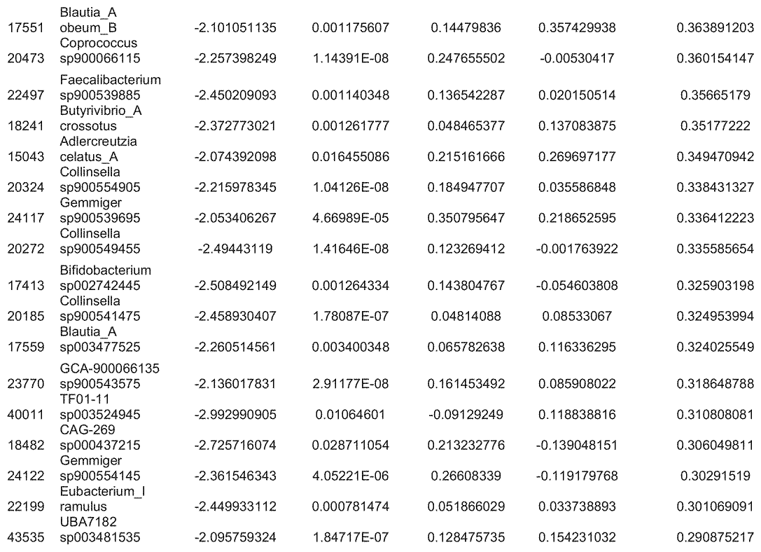

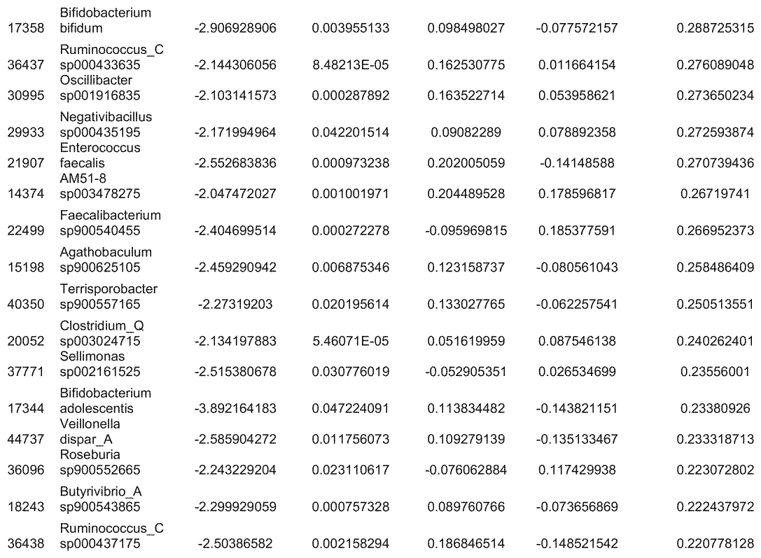

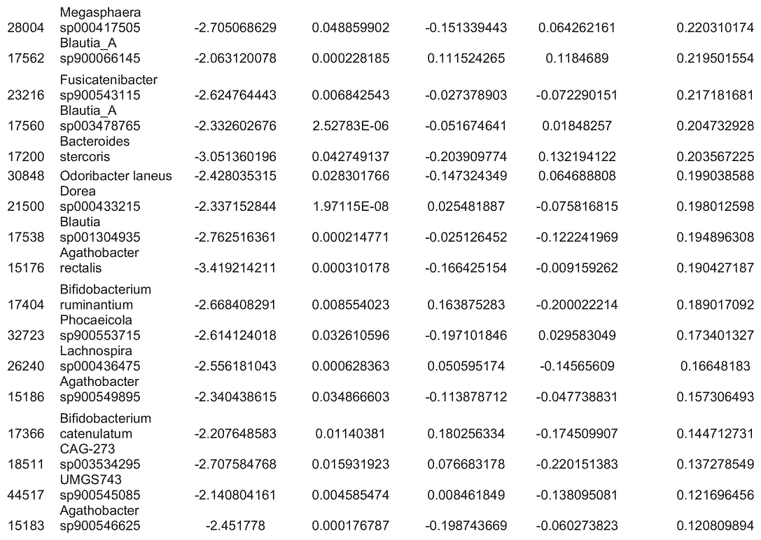

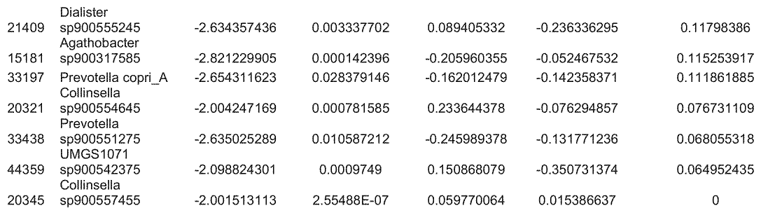

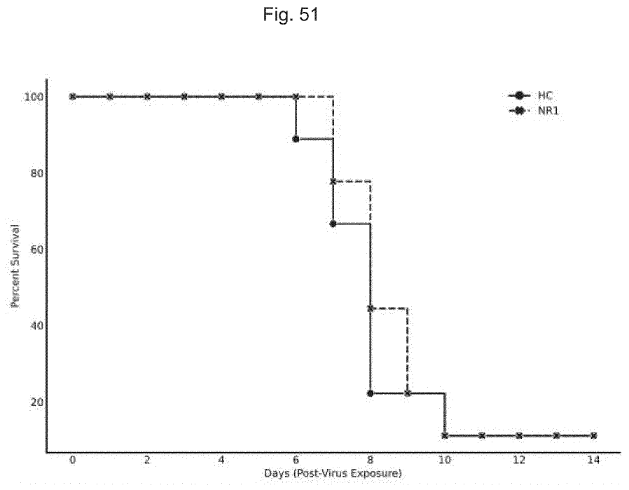

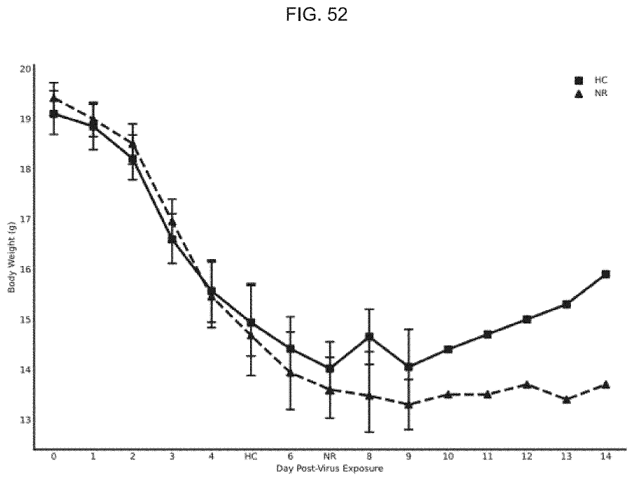

Nine of eighteen mice per group are inspected for viability and weighed daily for fourteen days after the date of viral infection. Mice from all four groups remained viable until day six post infection, after which mice from the infected groups start to succumb (FIG. 51). The daily percent survival of mice in the two infected groups (HC-FMT or NR-FMT) steadily decreased until day 10; mice are either found dead upon inspection or are humanely euthanized due to their adverse physical condition. One of 9 mice (11%) given the HC-FMT survived the infection, and one of nine mice (11%) given the NR1-FMT survived the infection. No statistically significant differences in survival curves were observed. All mice in the two uninfected groups remain viable until final euthanasia at day fourteen (data not shown). Mouse body weights (FIG. 52) in the infected groups steadily decreased after the initial day of infection until day 7 post infection, corresponding to reduction of viability within the groups. After this time, mice treated with HC-FMT were observed to maintain higher body weights than mice treated with NR-FMT. No significant change in body weights is observed for mice in the two FMT-treated but uninfected groups.

Lung Pathology after Viral Infection

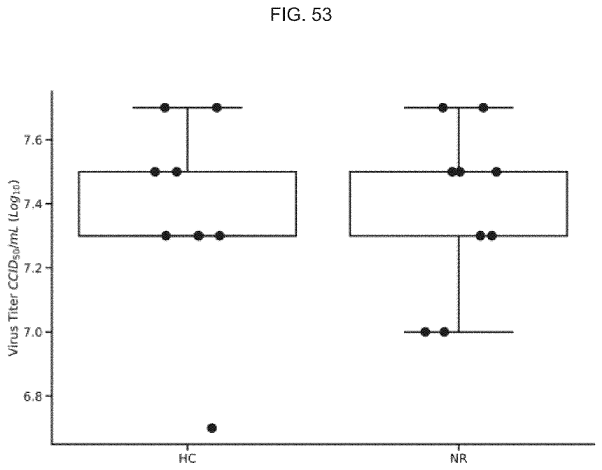

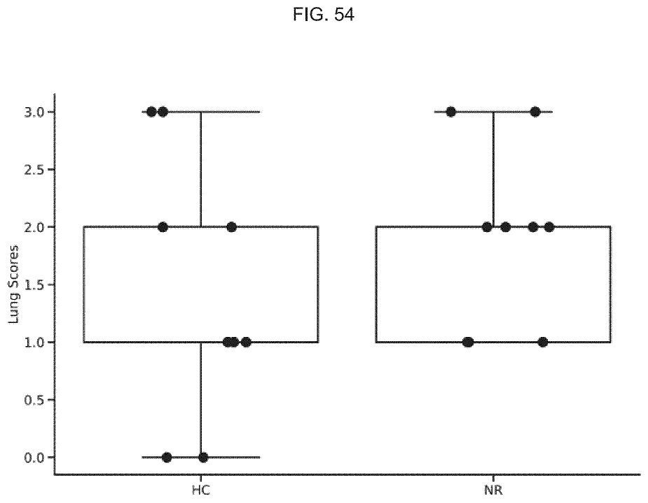

Three days after the day of viral infection, nine of eighteen mice from all four groups were sacrificed and their lungs removed for inspection and analyses. Virus titer was determined from homogenized mouse lung samples by end-point dilution in 96-well microplates of Madin Darby Canine Kidney (MDCK) cells. Briefly, log 10 dilutions of lung homogenate samples were prepared in minimum essential media (MEM) containing 10 units/ml trypsin, 1 μg/ml EDTA, and 50 μg/ml gentamicin (infection media). Confluent 96-well microplates of MDCK cells were prepared 24 hours prior to use and then washed to remove fetal bovine serum from the plates and replaced with infection media immediately prior to addition of lung homogenate dilutions. The plates were incubated for 6 days in a 37° C. incubator with 5% CO₂ and evaluated by visual observation of cytopathic effect (CPE). A 50% cell culture infectious dose (CCID₅₀) was calculated using the Reed-Muench method. No significant difference was observed by Welch's t-test for viral titers determined for lungs from infected HC-FMT or NR-FMT treated mice (FIG. 53), while lungs from uninfected mice were not analyzed for viral titer. Visual scores of lung pathogenesis also did not differ significantly between the two infected groups (FIG. 54), with little or no differences observed in degree of pathogenesis. Lungs removed from uninfected mice are all judged to have a “zero”, or healthy, lung score evaluation (data not shown). The weight of lungs removed from infected mice treated with NR-FMT were as a group higher than infected mice treated with HC-FMT (FIG. 55). Higher relative lung weights in infected NR-FMT mice than infected HC-FMT mice could reflect higher levels of inflammation in the lungs of mice in the NR-FMT treated group (Hurst, B. L., Evans, W. J., Smee, D. F., Van Wettere, A. J. & Tarbet, E. B. Evaluation of antiviral therapies in respiratory and neurological disease models of Enterovirus D68 infection in mice. Virology 526, 146-154 (2019)).

### Cytokine Concentrations in Lung Tissue

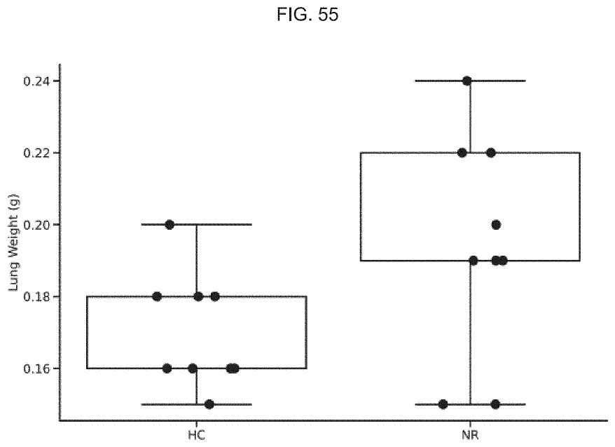

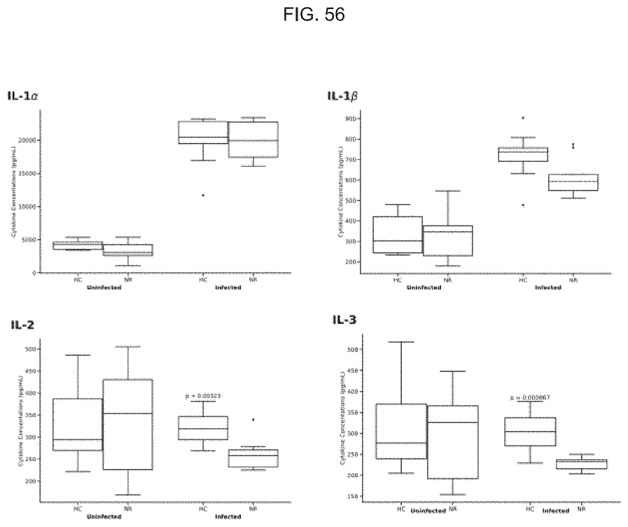

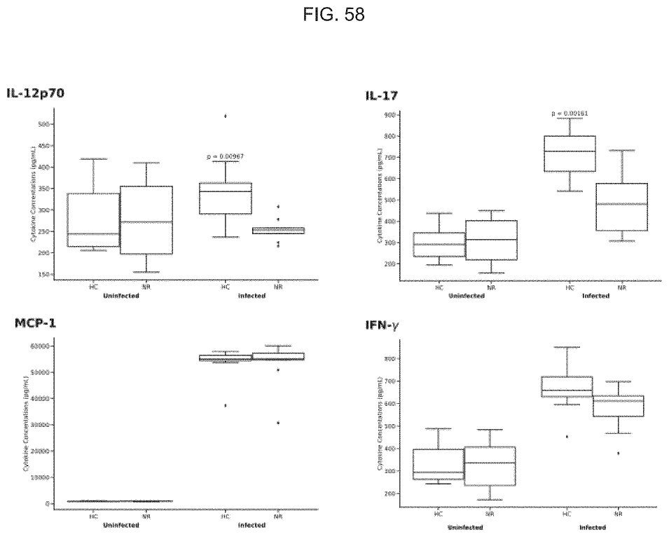

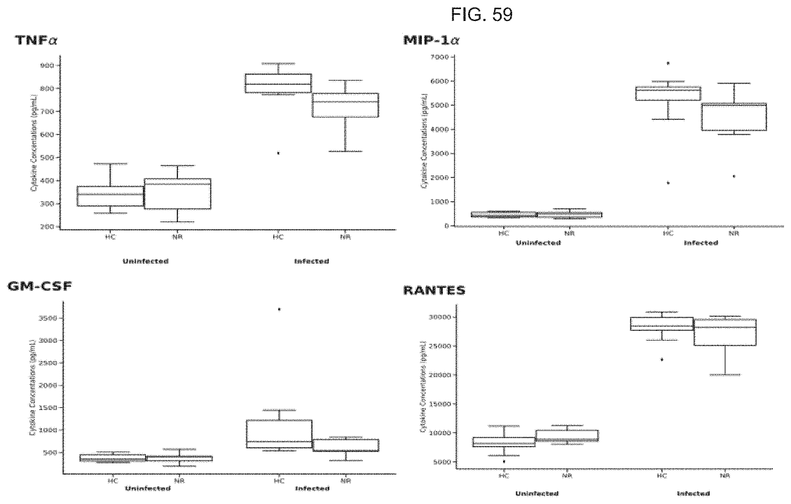

Lung tissue from FMT-treated, infected or uninfected mice, are subjected to analysis for concentrations of a variety of cytokines that are relevant immunological markers for viral infection and anti-tumor response. Lung homogenates were evaluated for concentrations of the following cytokines using a multi-plex cytokine kit (Quansys Biosciences, Logan, Utah) according to the manufacturer's instructions: Interleukins (IL-1α, IL-1β, IL-2, IL-3, IL-4, IL-5, IL-6, IL-10, IL-12p70, IL-17), Monocyte Chemoattractant Protein 1 (MCP-1), Interferon gamma (IFN-γ), Tumor Necrosis Factor alpha (TNFα), Macrophage Inflammatory Protein 1α (MIP-1α), Granulocyte-Macrophage Colony-Stimulating-Factor (GM-CSF), and Regulated on Activation, Normal T-cell Expressed and Secreted (RANTES). Concentrations of each of these cytokines in all four treatment groups are presented in FIGS. 56-59.

Concentrations of IL-1α, IL-1β, IL-6, Il-17, MCP-1, IFN-g, TNFα, MIP-1a, and RANTES are significantly increased in influenza-infected mice compared to uninfected mice, as expected as a result of influenza infection (Khanna, M., Rajput, R., Kumar, B., Kumari, A. & Saxena, L. Influenza virus Induced Cytokine Responses: An Evaluation of Host-Pathogen Association. (2014); Brydon, E. W. A., Morris, S. J. & Sweet, C. Role of apoptosis and cytokines in influenza virus morbidity. FEMS Microbiology Reviews 29, 837-850 (2005)). The cytokines/chemokines IL-12p70, IL-2, TNFα, and IFN-γ are all increased in infected HC-FMT treated mice compared to infected NR-FMT treated mice, and are indicative of a T helper cell 1 (Th1) type of immune response. IL-12p70 is one of two subunits of the heterodimeric cytokine IL-12. IL-12 is secreted by antigen presenting cells like dendritic cells to differentiate naïve T cells to Th1 type helper T cells, that then secrete IL-2, TNFα, and IFN-γ as a result (Zundler, S. & Neurath, M. F. Interleukin-12: Functional activities and implications for disease. Cytokine and Growth Factor Reviews 26, 559-568 (2015)). IL-12 mediated immune responses resulting from viral infection are critical for enhancing cytotoxic activity of Natural Killer (NK) cells and CD8+ cytotoxic T lymphocytes, and IL-12 induced INF-g can switch Th17 type T helper cells to Th-1 T helper cells against virus infected cells (Guo, Y., Cao, W. & Zhu, Y. Immunoregulatory functions of the IL-12 family of cytokines in antiviral systems. Viruses 11, (2019)). These results suggest that the NR-FMT treatment of infected mice comparatively inhibits proper Th1 type immunoresponses.

The cytokines IL-3, IL-4 and IL-5 are both increased in concentration in lungs from HC-FMT treated mice compared to lungs from NR-FMT treated mice. IL-4 and IL-5 are characteristic of a Th2 type of immunoresponse, as IL-4 helps differentiate naïve T cells to Th2 type T helper cells; IL-4 is then produced by differentiated Th2 type cells to autostimulate their own proliferation. IL-5 produced by Th2 cells encourages B cells to produce IgA against gastrointestinal infections. IL-3 is produced by both Th1 and Th2 cells, and promotes neutrophil production which are among the first innate immune cells to be recruited during viral infection (Lamichhane, P. P. & Samarasinghe, A. E. The Role of Innate Leukocytes during Influenza Virus Infection. J. Immunol. Res. 2019, (2019)).

The cytokine IL-17 is significantly elevated in lungs of infected HC-FMT treated mice compared to infected NR-FMT treated mice. IL-17 is produced by Th17 helper T cells after maturation in response to costimulation by IL-6 and Tumor Growth Factor beta (TGFβ, not measured in this study) produced from Dentritic Cells. IL-17 induces production of IL-6, GM-CSF and IL-1β, all three of which are shown to be elevated to different levels in infected HC-FMT mice. IL-17 hinders viral infection by enhancing Th1 type immune responses, and has a critical role in activation and survival of CD8+ cytotoxic T cells, as well as B cell maturation and migration into lung in response to influenza infection (Ma, W. T., et al. The protective and pathogenic roles of IL-17 in viral infections: Friend or foe? Open Biology 9, (2019)).

Both IL-3 and GM-CSF stimulation are negatively associated with Severe Acute Respiratory Illness (SARI) due to severe influenza disease (Wong, S.-S. et al. Severe Influenza Is Characterized by Prolonged Immune Activation: Results From the SHIVERS Cohort Study, doi:10.1093/infdis/jix571), although GM-CSF can itself be a marker for inflammation (Hamilton, J. A. Cytokines Focus GM-CSF in inflammation. Journal of Experimental Medicine 217, (2019)). GM-CSF is itself elevated in lungs of infected HC-FMT treated mice compared to infected NR-FMT treated mice and is important for stimulating production of granulocytes like neutrophils, eosinophils and basophils, as well as monocytes that go on to mature into macrophages and dendritic cells in infected tissues (Hamilton, J. A. Cytokines Focus GM-CSF in inflammation. Journal of Experimental Medicine 217, (2019)).

IL-1β is elevated in lungs of infected HC-FMT treated mice compared to lungs from infected NR-FMT treated mice. IL-1β is a pyrogen, fever producer and a master proinflammatory cytokine. It is induced in lung upon influenza infection, along with IL-6, IL-12, and TNFα (Kim, K. S., et al. Induction of interleukin-1 beta (IL-1β) is a critical component of lung inflammation during influenza A (H1N1) virus infection. J. Med. Virol. 87, 1104-1112 (2015)), and has been demonstrated in IL-1β receptor knock out mice to be important for survival after influenza infection (Schmitz, N., Kurrer, M., Bachmann, M. F. & Kopf, M. Interleukin-1 Is Responsible for Acute Lung Immunopathology but Increases Survival of Respiratory Influenza Virus Infection. J. Virol. 79, 6441-6448 (2005)). MIP-1α is elevated in infected HC-FMT treated mice compared to infected NR-FMT treated mice. MIP-1α is a chemotactic cytokine secreted by macrophages and is important in recruiting inflammatory cells to infection sites and in maintaining effector immune responses (Bhavsar, I., Miller, C. S. & A1-Sabbagh, M. Macrophage Inflammatory Protein-1 Alpha (MIP-1 alpha)/CCL3: As a biomarker. in General Methods in Biomarker Research and their Applications 1-2, 223-249 (Springer International Publishing, 2015). MIP-1α is produced in response to IL-12, and is important for NK cell response to influenza infection (Kay, A. W. et al. Enhanced natural killer-cell and T-cell responses to influenza A virus during pregnancy, doi:10.1073/pnas.1416569111).

### Comparison of Microbiobial Taxa of Donor Feces Used to Produce HC-FMT and NR-FMT

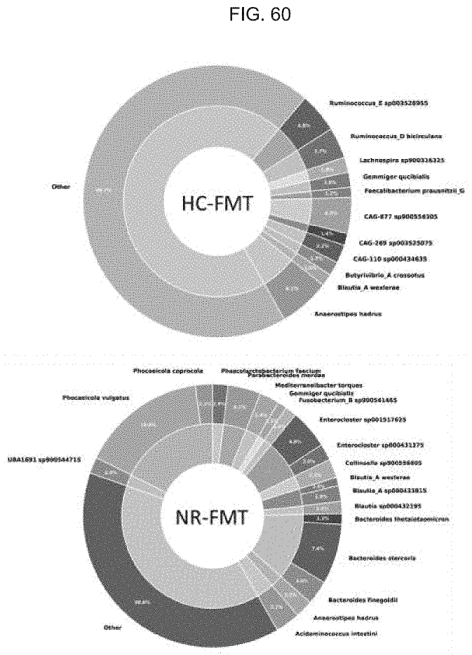

Whole genome sequencing was performed on the donor FMT samples as described in Example 24. The bacterial populations differ significantly in HC and NR fecal samples (FIG. 60). The microbial population within the fecal sample used to produce the HC-FMT is dominated by Gram-positive Ruminocococcaceae famil genera and species, Clostridiaceae family species like CAG-269 and CAG-110, as well as Lachnospiraceae family bacteria such as Faecalibacterium prausnitzii, Blautia wexlerae, and Anaerostipes hadrus. Lachnospiraceae and Ruminococcaceae family bacteria produce fermentation products like short chain fatty acids (SCFAs) that exert anti-inflammatory effects on the immune system (Pascal, M. et al. Microbiome and allergic diseases. Frontiers in Immunology 9, (2018)). Conversely, the microbial population within the NR donor fecal sample is more dominated by Gram-negative species like Bacteroides that produce lipopolysaccharides that can promote inflammation (Hakansson, A. & Molin, G. Gut microbiota and inflammation. Nutrients 3, 637-687 (2011)), and Fusobacterium species that associate with gastrointestinal disorders like Irritable Bowel Disease (IBD) (Liu, L. et al. Fusobacterium nucleatum Aggravates the Progression of Colitis by Regulating M1 Macrophage Polarization via AKT2 Pathway. Front. Immunol. 10, 1324 (2019)) and is considered a causal agent for colorectal cancer (Wu, J., Li, Q. & Fu, X. Fusobacterium nucleatum Contributes to the Carcinogenesis of Colorectal Cancer by Inducing Inflammation and Suppressing Host Immunity. Translational Oncology 12, 846-851 (2019)).

### References (Example 9)

[1] A. M. Evans, B. R. Bridgewater, Q. Liu, M. W. Mitchell, R. J. Robinson, H. Dai, S. J. Stewart, C. D. DeHaven, and L. A. D. Miller, High resolution mass spectrometry improves data quantity and quality as compared to unit mass resolution mass spectrometry in high-throughput profiling metabolomics. Metabolomics 4 (2014).
[2] C. D. DeHaven, A. M. Evans, H. Dai, and K. A. Lawton, Organization of GC/MS and LC/MS metabolomics data into chemical libraries. Journal of cheminformatics 2 (2010) 9.
[3] W. X. Zong, J. D. Rabinowitz, and E. White, Mitochondria and Cancer. Mol Cell 61 (2016) 667-676.
[4] N. M. Anderson, P. Mucka, J. G. Kern, and H. Feng, The emerging role and targetability of the TCA cycle in cancer metabolism. Protein Cell 9 (2018) 216-237.
[5] T. Li, and A. Le, Glutamine Metabolism in Cancer. Adv Exp Med Biol 1063 (2018) 13-32.
[6] D. Xiao, L. Zeng, K. Yao, X. Kong, G. Wu, and Y. Yin, The glutamine-alpha-ketoglutarate (AKG) metabolism and its nutritional implications. Amino Acids 48 (2016) 2067-80.
[7] L. Andrisic, D. Dudzik, C. Barbas, L. Milkovic, T. Grune, and N. Zarkovic, Short overview on metabolomics approach to study pathophysiology of oxidative stress in cancer. Redox Biol 14 (2018) 47-58.
[8] J. M. Estrela, A. Ortega, and E. Obrador, Glutathione in cancer biology and therapy. Crit Rev Clin Lab Sci 43 (2006) 143-81.
[9] R. F. Schwabe, and C. Jobin, The microbiome and cancer. Nat Rev Cancer 13 (2013) 800-12.
[10] A. P. Bhatt, M. R. Redinbo, and S. J. Bultman, The role of the microbiome in cancer development and therapy. CA Cancer J Clin 67 (2017) 326-344.
[11] M. Goudarzi, T. D. Mak, J. P. Jacobs, B. H. Moon, S. J. Strawn, J. Braun, D. J. Brenner, A. J. Fornace, Jr., and H. H. Li, An Integrated Multi-Omic Approach to Assess Radiation Injury on the Host-Microbiome Axis. Radiat Res 186 (2016) 219-34.

A number of embodiments of the invention have been described. Nevertheless, it can be understood that various modifications may be made without departing from the spirit and scope of the invention. Accordingly, other embodiments are within the scope of the following claims.

1: A method for:
controlling, ameliorating, lessoning the symptoms of or the mortality of a viral infection,
treating a viral infection, in an individual in need thereof,
enhancing the efficacy of an anti-viral drug or anti-viral treatment, or
enhancing the efficacy of a vaccine, or changing the gut microbiome in an individual in need thereof such that the individual has fewer or diminished side effects or negative reactions to an administered vaccine,
wherein optionally the vaccine is an antiviral vaccine, and optionally the antiviral vaccine is an RNA-based vaccine, wherein optionally the RNA-based vaccine comprises RNA formulated in a liposome or a nanolipid particle,
the method comprising:
(a) administering or having administered to an individual in need thereof a formulation comprising at least two different species or genera or types of non-pathogenic bacteria,
wherein each of the non-pathogenic bacteria comprise or are in the form of a plurality of non-pathogenic colony forming live bacteria, a plurality of non-pathogenic germinable bacterial spores, or a combination thereof; or,
(b) (i) providing a formulation comprising at least two different species or genera or types of non-pathogenic bacteria, wherein each of the non-pathogenic bacteria comprise or are in the form of a plurality of non-pathogenic colony forming live bacteria, a plurality of non-pathogenic germinable bacterial spores, or a combination thereof, and
(ii) administering or having administered to an individual in need thereof the formulation;
wherein the formulation comprises a or any combination of at least two different species or genera of non-pathogenic, live bacteria, or spore thereof, if the bacteria is spore forming, as described Tables 1, 4, 7, 8 and/or 42, or live biotherapeutic compositions or combinations of bacteria as set forth in Tables 9 and/or 42,
and optionally the different species or genera or types of non-pathogenic, live bacteria are present in approximately equal amounts, or each of the different species or genera or types of non-pathogenic, live bacteria or non-pathogenic germinable bacterial spores represent at least about 1%, 5%, 10%, 20%, 30%, 40%, or 50% or more, or between about 1% and 75%, of the total amount of non-pathogenic, live bacteria and non-pathogenic germinable bacterial spores in the formulation,
and optionally only or substantially only non-pathogenic, live bacteria are present in the formulation, or only or substantially only non-pathogenic germinable bacterial spores are present in the formulation, or approximately equal amounts of non-pathogenic, live bacteria and non-pathogenic germinable bacterial spores are present in the formulation.

2: The method of claim 1, further comprising administering or having administered one or any one of: an anti-viral treatment or drug, an immune checkpoint inhibitor, a Chimeric Antigen Receptor (CAR) T-cell therapy (CAR-T) or an immunotherapy or an immune-enhancing therapy or a combination thereof,
and optionally the anti-viral treatment or drug comprises administration of: an antimicrobial drug, optionally an antiviral, antibacterial or antimalarial, drug, and optionally the antimicrobial or antiviral drug comprises one or any one of: lopinavir; ritonavir; oseltamivir (for example, TAMIFLU™); lopinavir combined (formulated) with ritonavir, or KALETRA™; chloroquine phosphate (for example, RESOCHIN™), chloroquine diphosphate, hydroxychloroquine (for example, PLAQUENIL™) or oral chloroquine (for example, ARALEN™); remdesivir (for example, GS-5734™, Gilead Sciences); nevirapine, efavirenz, emtricitabine, tenofovir (or the combination efavirenz with emtricitabine and tenofovir, or ATRIPLA™); amprenavir (for example, Agenerase™); nelfinavir (for example, VIRACEPT™); a thiazolide class drug, optionally nitazoxanide (or Alinia™, Nizonide™) or tizoxanide (or 2-Hydroxy-N-(5-nitro-2-thiazolyl)benzamide); plitidepsin (also known as dehydrodidemnin B), or APLIDIN™ (PharmaMar, S.A.); an inhibitor or S-phase kinase-associated protein 2 (SKP2), or dioscin, or niclosamide, or Niclocide™, Fenasal™, or Phenasal™; ribavirin; an interferon such as interferon alpha, interferon beta, interferon type I, interferon type II and/or interferon type III, or a combination of ribavirin and interferon beta, or a combination of lopinavir and ritonavir and interferon-beta-1 b; abacavir, actemra, acyclovir for example, (Aciclovir™), adefovir, amantadine, ampligen, amprenavir (for example, Agenerase™), aprepitant, atazanavir, balavir, baloxavir marboxil (Xofluza™), bepotastine, bevirimat, bictegravir, biktarvy, brilacidin, cidofovir, caspofungin, lamivudine and zidovudine (for example, COMBVIR™), cobicstat, colisitin, cocaine, danoprevir or danoprevir and ritonavir (for example, GANOVO™) darunavir (or darunavir and cobicstat, for example, PREZCOBIX™), delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, ibacitabine, icatibant, idoxuridine, ifenprodil, imiquimod, imunovir, indinavir, inosine, lamivudine, lopinavir, loviride, ledipasvir, leronlimab, maraviroc, methisazone, moroxydine, nelfinavir, nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (for example, BCX4430 by Biocryst, Immucillin-A™), remdesivir (for example, GS-5734™, Gilead Sciences), cytarabine, gemcitabine, emtricitabine, zalcitabine, stavudine, telbivudine, zidovudine, idoxuridine and/or trifluridine or any combination thereof), oseltamivir (or TAMIFLU™), peginterferon alfa-2a, penciclovir, peramivir (for example, Rapivab™) perfenazine, pleconaril, plurifloxacin, podophyllotoxin, pyramidine, raltegravir, rifampicin, ribavirin, rilpivirine, rimantadine, ritonavir, saquinavir, sofosbuvir, telaprevir, tegobuv, tenofovir alafenamide, tenofovir disoproxil, tenofovir, tipranavir, trifluridine, trizivir, tromantadine, truvada, valaciclovir (for example, Valtrex™) valganciclovir, valrubicin, vapreotide, vicriviroc, vidarabine, viramidine, velpatasvir, vivecon, zalcitabine, zanamivir (for example, Relenza™) or zidovudine; a serine protease inhibitor, optionally camostat; an anti-PD-1 checkpoint inhibitor, optionally camrelizumab; a compound or antibody capable of binding complement factor C5 and blocking membrane attack complex formation, optionally eculizumab; a cathepsin inhibitor, optionally a cathepsin K, B or L inhibitor, optionally relacatib; thalidomide, or thalidomide and glucocorticoid (optionally low-dose glucocorticoid), or and thalidomide and celecoxib; an antibacterial antibiotic or a macrolide drug, wherein optionally the macrolide drug comprises azithromycin (for example, Zithromax™, or Azithrocin™), clarithromycin (for example, Biaxin™), erythromycin (for example, Erythrocin™), or fidaxomicin (for example, Dificid™ or Dificlir™) troleandomycin (for example, Tekmisin™), tylosin (for example, Tylocine™ or Tylan™), solithromycin (for example, Solithera™), oleandomycin (or Sigmamycine™), midecamycin, roxithromycin, kitasamycin or turimycin, josamycin, carbomycin or magnamycin, and/or spiramycin; opaganib or YELIVA™—an anti-interleukin-6 antibody (e.g., tocilizumab or tocilizumab and favipiravir, for example, ACTEMRA™); sarilumab (for example, KEVZARA™); umifenovir (for example, ARBIDOL™); colchicine, or Colcrys™, Mitigare™; a corticosteroid class drug such as budesonide (or RHINOCORT™ or PULMICORT™), prednisolone (or ORAPRED™), methyl-prednisolone, prednisone (or DELTASONE™ or ORASONE™) or hydrocortisone (or CORTEF™); an anti-androgen drug, or bicalutamide; a hydrocortisone or cortisol (or CORTEF™, SOLUCORTEF™), or hydrocortisone sodium succinate or hydrocortisone acetate or dexamethasome (or Dextenza™, Ozurdex™, Neofordex™); famotidine, or PEPCID™ an antihistamine class drug such as azelastine, or Astelin™, Optivar™, Allergodil™, brompheniramine, fexofenadine or ALLEGRA™, pheniramine or AVIL™, or chlorpheniramine; a dendrimer, or an astodrimer sodium (Starpharma, Melbourne, Australia); a selective serotonin reuptake inhibitor (SSRI) class drug, optionally fluvoxamine, or Luvox™, Faverin™, Fluvoxin™; a nicotinic antagonist, a dopamine agonist or a noncompetitive N-Methyl-d-aspartic acid or N-Methyl-d-aspartate (NMDA) antagonist; an immunosuppressive drug, or tocilizumab or atlizumab, or Actemra™, or RoActemra™, or a calcineurin inhibitor (CNI), or ciclosporin or cyclosporine or cyclosporin); or, any two, three or more or combination thereof;
and optionally the anti-viral treatment or drug, the immune checkpoint inhibitor, the Chimeric Antigen Receptor (CAR) T-cell therapy (CAR-T) or the immunotherapy, or the combination thereof, is administered before, during (concurrently with) and/or after administration the formulation.

3: The method of claim 1, or claim 2 wherein:
(a) the formulation comprises an inner core surrounded by an outer layer of polymeric material enveloping the inner core,
wherein the non-pathogenic bacteria or the non-pathogenic germinable bacterial spores are substantially in the inner core, and optionally the polymeric material comprises a natural polymeric material;
(b) the formulation is formulated or manufactured as or in: a nano-suspension delivery system; an encochleated formulation; or, as a multilayer crystalline, spiral structure with no internal aqueous space;
(c) the formulation is formulated or manufactured as a delayed or gradual enteric release composition or formulation,
and optionally the formulation comprises a gastro-resistant coating designed to dissolve at a pH of 7 in the terminal ileum,
optionally an active ingredient is coated with an acrylic based resin or equivalent, optionally a poly(meth)acrylate, optionally a methacrylic acid copolymer B, NF, optionally Eudragit S™ (Evonik Industries AG, Essen, Germany), which dissolves at pH 7 or greater, optionally comprises a multimatrix (MMX) formulation,
and optionally the MMX formulation is manufactured as enteric coated to bypass the acid of the stomach and bile of the duodenum.

4: The method of claim 1, wherein the plurality of non-pathogenic colony forming live bacteria are substantially dormant colony forming live bacteria, or the plurality of non-pathogenic colony forming live bacteria or the plurality of non-pathogenic germinable bacterial spores are lyophilized,
wherein optionally the dormant colony forming live bacteria comprise live vegetative bacterial cells that have been rendered dormant by lyophilization or freeze drying.

5: The method of claim 1, wherein the formulation comprises at least about 1×104 colony forming units (CFUs), or between about 1×10¹ and 1×10¹³ CFUs, 1×10² and 1×10¹⁰ CFUs, 1×10¹ and 1×10⁸ CFUs, 1×10³ and 1×10⁷ CFUs, or 1×10⁴ and 1×10⁶ CFUs, of non-pathogenic live bacteria and/or non-pathogenic germinable bacterial spores.

6: The method of claim 1, wherein the formulation comprises at least one or any one, several, or all of non-pathogenic bacteria or spore of the family or genus or class: Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), or a combination thereof.

7: The method of claim 1, wherein the formulation;
(a) comprises at least one or any one, several, or all of non-pathogenic bacteria or spore form thereof as set forth in Tables 1, 4, 7 or 8, or included in the combination of non-pathogenic bacteria and/or spores thereof (or spore derived from) as set forth in Table 9 and/or Table 42;
(b) the formulation comprises combination of non-pathogenic bacteria and/or spores thereof (or spore derived from) as set forth in Table 9 and/or Table 42;
(c) the formulation comprises water, sterile water, saline, sterile saline, a pharmaceutically acceptable preservative, a carrier, a buffer, a diluent, an adjuvant or a combination thereof;
(d) the formulation is administered orally or rectally, or is formulated and/or administered as a liquid, a food, a gel, a candy, an ice, a lozenge, a tablet, pill or capsule, or a suppository or as an enema formulation, or the formulation is administered as an or is in a form for intra-rectal or intra-colonic administration;
(e) the formulation is administered to the individual in need thereof in one, two, three, or four or more doses, and wherein the one, two, three, or four or more doses are administered on a daily basis,
optionally once a day, bid or tid), every other day, every third day, or about once a week, and optionally the two, three, or four or more doses are administered at least a week apart, or dosages are separated by about a week; or
(f) further comprises an antibiotic, or the method further comprises administration of an antibiotic,
and optionally at least one dose of the antibiotic is administered before a first administration of the formulation, optionally at least one dose of the antibiotic is administered one day or two days, or more, before a first administration of the formulation.

8-12. (canceled)

13: The method of claim 1, wherein the inhibitor of the inhibitory immune checkpoint molecule:
(a) comprises a protein or polypeptide that binds to an inhibitory immune checkpoint protein, and optionally inhibitor of the inhibitory immune checkpoint protein is an antibody or an antigen binding fragment thereof that specifically binds to the inhibitory immune checkpoint protein;
(b) targets a compound or protein comprising: a CTLA4 or CTLA-4 (cytotoxic T-lymphocyte-associated protein 4, also known as CD152, or cluster of differentiation 152); Programmed cell Death protein 1, also known as PD-1 or CD279; Programmed Death-Ligand 1 (PD-L1), also known as cluster of differentiation 274 (CD274) or B7 homolog 1 (B7-H1)): PD-L2: A2AR (adenosine A2A receptor, also known as ADORA2A); B7-H3; B7-H4; BTLA (B- and T-lymphocyte attenuator protein); KIR (Killer-cell Immunoglobulin-like Receptor); IDO (Indoleamine-pyrrole 2,3-dioxygenase); LAG3 (Lymphocyte-Activation Gene 3 protein); TIM-3; VISTA (V-domain Ig suppressor of T cell activation protein); or any combination thereof;
(c) comprises: ipilimumab or Yervoy®; pembrolizumab or Keytruda®; nivolumab or OPDIVO®; atezolizumab or TECENTRIP®; avelumab or BAVENCIO®; durvalumab or IMFINZI®; AMP-224 (MedImmune), AMP-514 (an anti-programmed cell death 1 (PD-1) monoclonal antibody (mAb) (MedImmune)), PDR001 (a humanized mAb that targets PD-1), STI-A1110 or STI-A1010 (Sorrento Therapeutics), BMS-936559 (Bristol-Myers Squibb), BMS-986016 (Bristol-Myers Squibb), TSR-042 (Tesaro), JNJ-61610588 (Janssen Research & Development), MSB-0020718C, AUR-012, enoblituzumab (also known as MGA271) (MacroGenics, Inc.), MBG453, LAG525 (Novartis), BMS-986015 (Bristol-Myers Squibb), or any combination thereof; or
(d) or the stimulatory immune checkpoint molecule, is administered by: intravenous (IV) injection, intramuscular (IM) injection, intratumoral injection or subcutaneous injection; or, is administered orally or by suppository; or the formulation further comprises at least one immune checkpoint inhibitor.

14-16. (canceled)

17: The method of claim 1, wherein the cancer is melanoma, advanced melanoma, cutaneous or intraocular melanoma, primary neuroendocrine carcinoma of the skin, breast cancer, a cancer of the head and neck, uterine cancer, rectal and colorectal cancer, a cancer of the head and neck, cancer of the small intestine, a colon cancer, a cancer of the anal region, a stomach cancer, lung cancer, brain cancer, non-small-cell lung cancer, ovarian cancer, angiosarcoma, bone cancer, osteosarcoma, prostate cancer; cancer of the bladder; cancer of the kidney or ureter or renal cell carcinoma, or carcinoma of the renal pelvis; a neoplasm of the central nervous system (CNS) or renal cell carcinoma.

18: The method of claim 1, comprising, or further comprising, administering, or having administered, or delivering, a genetically (or recombinantly) engineered cell,
wherein optionally the genetically engineered cell is: a microbe or spore derived from a microbe as used in a method of any of the preceding claims, or a method of claims 1 to 17; or, a non-pathogenic bacteria or spore form thereof as set forth in Tables 1, 4, 7 or 8; or, a non-pathogenic bacteria or spore form thereof included in a combination of non-pathogenic bacteria and/or spores thereof (or spore derived from) as set forth in Table 9 and/or Table 42,
and optionally the microbe is genetically engineered to express or secrete a heterologous or overexpress an endogenous immunomodulatory molecule, and optionally the immunomodulatory molecule is an immunomodulatory protein or peptide, and optionally the immunomodulatory molecule is an immunostimulatory molecule,
and optionally the microbe is genetically engineered to overexpress a pathway for production of at least one short chain fatty acid (SCFA), and optionally the SCFA comprises butyrate or butyric acid, propionate or acetate,
and optionally the microbe is genetically engineered by inserting a heterologous nucleic acid into the microbe, and optionally the heterologous nucleic acid encodes an exogenous membrane protein,
and optionally the immunostimulatory molecule, protein or peptide comprises a non-specific immunostimulatory protein, and optionally the non-specific immunostimulatory protein comprises a cytokine, and optionally the cytokine comprises an interferon (optionally an IFN-α2a, IFN-α2b), and interleukin (optionally IL-2, IL-4, IL-7, IL-12), an interferon (IFN), a TNF-α, a granulocyte colony-stimulating factor (G-CSF, also known as filgrastim, lenograstim or Neupogen®), a granulocyte monocyte colony-stimulating factor (GM-CSF, also known as molgramostim, sargramostim, Leukomax®, Mielogen® or Leukine®), or any combination thereof,
and optionally the immunostimulatory molecule, protein or peptide comprises a specific immunostimulatory protein or peptide, and optionally the specific immunostimulatory protein or peptide comprises an immunogen that can generate a specific humeral or cellular immune response or an immune response to a cancer antigen,
and optionally the genetically engineered cell is a lymphocyte, and optionally the genetically engineered cell expresses a chimeric antigen receptor (CAR), and optionally the lymphocyte is a B cell or a T cell (CAR-T cell), and optionally the lymphocyte is a tumor infiltrating lymphocyte (TIL),
and optionally the microbe is genetically engineered to substantially decrease, reduce or eliminate the microbe's toxicity,
and optionally the microbe is genetically engineered to comprise a kill switch so the microbe can be rendered non-vital after administration of an appropriate trigger or signal,
and optionally the microbe is genetically engineered to secrete anti-inflammatory compositions or have an anti-inflammatory effect,
and optionally the genetically engineered cell is administered or delivered before administration of, simultaneously with, and/or after administration or delivery of the formulation.

19: A formulation or a pharmaceutical composition comprising:
(a) a combination of microbes as set forth in Table 9 and/or Table 42;
(b) a combination of microbes as used in claim 1; and/or
(c) at least two different species or genera (or types) of non-pathogenic bacteria, wherein each of the non-pathogenic bacteria comprise (or are in the form of) a plurality of non-pathogenic colony forming live bacteria, a plurality of non-pathogenic germinable non-pathogenic bacterial spores, or a combination thereof, and the formulation comprises at least one (or any one, several, or all of) non-pathogenic bacteria or spore of the family or genus (or class): Agathobaculum (TaxID: 2048137), Alistipes (TaxID: 239759), Anaeromassilibacillus (TaxID: 1924093), Anaerostipes (TaxID: 207244), Asaccharobacter (TaxID: 553372), Bacteroides (TaxID: 816), Barnesiella (TaxID: 397864), Bifidobacterium (TaxID: 1678), Blautia (TaxID: 572511), Butyricicoccus (TaxID: 580596), Clostridium (TaxID: 1485), Collinsella (TaxID: 102106), Coprococcus (TaxID: 33042), Dorea (TaxID: 189330), Eubacterium (TaxID: 1730), Faecalibacterium (TaxID: 216851), Fusicatenibacter (TaxID: 1407607), Gemmiger (TaxID: 204475), Gordonibacter (TaxID: 644652), Lachnoclostridium (TaxID: 1506553), Methanobrevibacter (TaxID: 2172), Parabacteroides (TaxID: 375288), Romboutsia (TaxID: 1501226), Roseburia (TaxID: 841), Ruminococcus (TaxID: 1263), Erysipelotrichaceae (TaxID: 128827), Coprobacillus (TaxID: 100883), Erysipelatoclostridium sp. SNUG30099 (TaxID: 1982626), Erysipelatoclostridium (TaxID: 1505663), or a combination thereof.

20: The formulation or a pharmaceutical composition of claim 19, wherein:
(a) the formulation comprises at least one (or any one, several, or all of) non-pathogenic bacteria or spore form thereof as set forth in Tables 1, 4, 7, 8 and/or 42, or included in the combination of non-pathogenic bacteria and/or spores thereof (or spore derived from) as set forth in Table 9 and/or Table 42;
(b) the formulation comprises an inner core surrounded by an outer layer of polymeric material enveloping the inner core, wherein the non-pathogenic bacteria or the non-pathogenic germinable bacterial spores are substantially in the inner core, and optionally the polymeric material comprises a natural polymeric material;
(c) the plurality of non-pathogenic colony forming live bacteria are substantially dormant colony forming live bacteria, or the plurality of non-pathogenic colony forming live bacteria or the plurality of non-pathogenic germinable bacterial spores are lyophilized,
wherein optionally the non-pathogenic dormant colony forming live bacteria comprise live vegetative bacterial cells that have been rendered dormant by lyophilization or freeze drying;
(d) the formulation comprises at least 1×104 colony forming units (CFUs), or between about 1×102 and 1×108 CFUs, 1×103 and 1×107 CFUs, or 1×104 and 1×106 CFUs, of live non-pathogenic bacteria and/or non-pathogenic germinable bacterial spores;
(e) the formulation or pharmaceutical composition comprises water, saline, a pharmaceutically acceptable preservative, a carrier, a buffer, a diluent, an adjuvant or a combination thereof;
(f) the formulation or pharmaceutical composition is formulated for administration orally or rectally, or is formulated as a liquid, a food, a gel, a geltab, a candy, a lozenge, a tablet, pill or capsule, or a suppository;
(g) the formulation or pharmaceutical composition further comprises or is formulated with: an anti-viral drug or drug combination or formulation, a biofilm disrupting or dissolving agent, an antibiotic, an inhibitor of an inhibitory immune checkpoint molecule and/or a stimulatory immune checkpoint molecule (or any composition for use in checkpoint blockade immunotherapy);
(h) further comprising or formulated with one or any one of: an anti-viral treatment or drug or combination of drugs, an immune checkpoint inhibitor, a Chimeric Antigen Receptor (CAR) T-cell therapy (CAR-T) or an immunotherapy (for example, an immune-enhancing therapy) or a combination thereof,
and optionally the anti-viral treatment or drug or combination of drugs comprises: an antimicrobial drug, optionally an antiviral, antibacterial or antimalarial, drug, and optionally the antimicrobial (optionally antiviral) drug comprises one or any one of: lopinavir; ritonavir; oseltamivir (for example, TAMIFLU™); lopinavir combined (formulated) with ritonavir, or KALETRA™: chloroquine phosphate (for example, RESOCHIN™), chloroquine diphosphate, hydroxychloroquine (for example, PLAQUENIL™) or oral chloroquine (for example, ARALEN™); remdesivir (for example, GS-5734™, Gilead Sciences); nevirapine, efavirenz, emtricitabine, tenofovir (or the combination efavirenz with emtricitabine and tenofovir, or ATRIPLA™); amprenavir (for example, Agenerase™); nelfinavir (for example, VIRACEPT™); a thiazolide class drug, optionally nitazoxanide (or Alinia™, Nizonide™) or tizoxanide (or 2-Hydroxy-N-(5-nitro-2-thiazolyl)benzamide); plitidepsin (also known as dehydrodidemnin B), or APLIDIN™ (PharmaMar, S.A.); an inhibitor or S-phase kinase-associated protein 2 (SKP2), or dioscin, or niclosamide, or Niclocide™, Fenasal™, or Phenasa™: ribavirin; an interferon such as interferon alpha, interferon beta, interferon type I, interferon type II and/or interferon type III, or a combination of ribavirin and interferon beta, or a combination of lopinavir and ritonavir and interferon-beta-1b; abacavir, actemra, acyclovir for example, (Aciclovir™), adefovir, amantadine, ampligen, amprenavir (for example, Agenerase™), aprepitant, atazanavir, balavir, baloxavir marboxil (Xofluza™), bepotastine, bevirimat, bictegravir, biktarvy, brilacidin, cidofovir, caspofungin, lamivudine and zidovudine (for example, COMBVIR™), cobicstat, colisitin, cocaine, danoprevir or danoprevir and ritonavir (for example, GANOVO™) darunavir (or darunavir and cobicstat, for example, PREZCOBIX™), delavirdine, descovy, didanosine, docosanol, dolutegravir, ecoliever, edoxudine, efavirenz, elvitegravir, emtricitabine, enfuvirtide, entecavir, epirubicin, epoprostenol, etravirine, famciclovir, fomivirsen, fosamprenavi, foscarnet, fosfonet, ibacitabine, icatibant, idoxuridine, ifenprodil, imiguimod, imunovir, indinavir, inosine, lamivudine, lopinavir, loviride, ledipasvir, leronlimab, maraviroc, methisazone, moroxydine, nelfinavir, nevirapine, nexavir, nitazoxanide, norvir, a nucleoside analogue (optionally brincidofovir, didanosine, favipiravir (also known as T-705, avigan, or favilavir, Toyama Chemical, Fujifilm, Japan), vidarabine, galidesivir (for example, BCX4430 by Biocryst, Immucillin-A™), remdesivir (for example, GS-5734™, Gilead Sciences), cytarabine, gemcitabine, emtricitabine, zalcitabine, stavudine, telbivudine, zidovudine, idoxuridine and/or trifluridine or any combination thereof), oseltamivir (or TAMIFLU™), peginterferon alfa-2a, penciclovir, peramivir (for example, Rapivab™), perfenazine, pleconaril, plurifloxacin, podophyllotoxin, pyramidine, raltegravir, rifampicin, ribavirin, rilpivirine, rimantadine, ritonavir, saguinavir, sofosbuvir, telaprevir, tegobuv, tenofovir alafenamide, tenofovir disoproxil, tenofovir, tipranavir, trifluridine, trizivir, tromantadine, truvada, valaciclovir (for example, Valtrex™), valganciclovir, valrubicin, vapreotide, vicriviroc, vidarabine, viramidine, velpatasvir, vivecon, zalcitabine, zanamivir (for example, Relenza™) or zidovudine; a serine protease inhibitor, optionally camostat; an anti-PD-1 checkpoint inhibitor, optionally camrelizumab; a compound or antibody capable of binding complement factor C5 and blocking membrane attack complex formation, optionally eculizumab: a cathepsin inhibitor, optionally a cathepsin K, B or L inhibitor, optionally relacatib; thalidomide, or thalidomide and glucocorticoid (optionally low-dose glucocorticoid), or and thalidomide and celecoxib; an antibacterial antibiotic or a macrolide drug, wherein optionally the macrolide drug comprises azithromycin (for example, Zithromax™, or Azithrocin™), clarithromycin (for example, Biaxin™), erythromycin (for example, Erythrocin™), or fidaxomicin (for example, Dificid™ or Dificlir™), troleandomycin (for example, Tekmisin™), tylosin (for example, Tylocine™ or Tylan™), solithromycin (for example, Solithera™), oleandomycin (or Sigmamycine™), midecamycin, roxithromycin, kitasamycin or turimycin, iosamycin, carbomycin or magnamycin, and/or spiramycin; opaganib or YELIVA™ an anti-interleukin-6 antibody (e.g., tocilizumab or tocilizumab and favipiravir, for example, ACTEMRA™); sarilumab (for example, KEVZARA™); umifenovir (for example, ARBIDOL™); or, any two, three or more or combination thereof;
(i) the inhibitor of an inhibitory immune checkpoint molecule comprises a protein or polypeptide that binds to an inhibitory immune checkpoint protein, and optionally the inhibitor of the inhibitory immune checkpoint molecule is an antibody or an antigen binding fragment thereof that binds to an inhibitory immune checkpoint protein;
(j) the inhibitor of an inhibitory immune checkpoint molecule targets a compound or protein comprising: CTLA4 or CTLA-4 (cytotoxic T-lymphocyte-associated protein 4, also known as CD152, or cluster of differentiation 152): Programmed cell Death protein 1, also known as PD-1 or CD279; Programmed Death-Ligand 1 (PD-L1), also known as cluster of differentiation 274 (CD274) or B7 homolog 1 (B7-H1)); PD-L2: A2AR (adenosine A2A receptor, also known as ADORA2A); B7-H3; B7-H4; BTLA (B- and T-lymphocyte attenuator protein); KIR (Killer-cell Immunoglobulin-like Receptor); IDO (Indoleamine-pyrrole 2,3-dioxygenase); LAG3 (Lymphocyte-Activation Gene 3 protein); TIM-3; VISTA (V-domain Ig suppressor of T cell activation protein) or any combination thereof;
(k) the inhibitor of an inhibitory immune checkpoint molecule comprises: ipilimumab or Yervoy®; pembrolizumab or Keytruda®; nivolumab or OPDIVO®; atezolizumab or TECENTRIP®; avelumab or BAVENCIO®; durvalumab or IMFINZI®; AMP-224 (MedImmune), AMP-514 (an anti-programmed cell death 1 (PD-1) monoclonal antibody (mAb) (MedImmune)), PDR001 (a humanized mAb that targets PD-1), STI-A1110 or STI-A1010 (Sorrento Therapeutics), BMS-936559 (Bristol-Myers Squibb), BMS-986016 (Bristol-Myers Squibb), TSR-042 (Tesaro), JNJ-61610588 (Janssen Research & Development), MSB-0020718C, AUR-012, enoblituzumab (also known as MGA271) (MacroGenics, Inc.), MBG453, LAG525 (Novartis), BMS-986015 (Bristol-Myers Squibb), or any combination thereof; or
(l) the stimulatory immune checkpoint molecule comprises a member of the tumor necrosis factor (TNF) receptor superfamily, optionally CD27, CD40, OX40, GITR (a glucocorticoid-Induced TNFR family Related gene protein) or CD137, or comprises a member of the B7-CD28 superfamily, optionally CD28 or Inducible T-cell co-stimulator (ICOS).

21-31. (canceled)

32: A kit or product of manufacture comprising a formulation or pharmaceutical composition of claim 19, wherein optionally the product of manufacture is an implant.

33-36. (canceled)
